(19) United States
(12) Patent Application Publication
(10) Pub. No.: US 2006/0264460 A1
(43) Pub. Date: Nov. 23, 2006

(54) 3-Cyanoquinoline inhibitors of Tpl2 kinase and methods of making and using the same

(75) Inventors: Neal Jeffrey Green, Newton, MA (US); Yonghan Hu, Acton, MA (US); Neelu Kaila, Lexington, MA (US); Kristin Marie Janz, Arlington, MA (US); Jennifer R. Thomason, Revere, MA (US); Huan-Qiu Li, Brighton, MA (US); Rajeev Hotchandani, Somerville, MA (US); Junjun Wu, Arlington, MA (US); Ariamala Gopalsamy, Mahwah, NJ (US); Steve Y. Tam, Wellesley, MA (US); Lih-Ling Lin, Concord, MA (US); John William Cuozzo, Natick, MA (US); Satenig Y. Guler, Watertown, MA (US); Adrian Huang, Lexington, MA (US); Jeffrey Scott condon, Melrose, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/436,485

(22) Filed: May 18, 2006

Related U.S. Application Data
(60) Provisional application No. 60/682,331, filed on May 18, 2005.

(51) Int. Cl. A61K 31/4709; A61K 31/4706; C07D 401/02; C07D 215/38

(52) U.S. Cl. 514/313; 546/159

(57) ABSTRACT

The present invention provides compounds of formula (I): 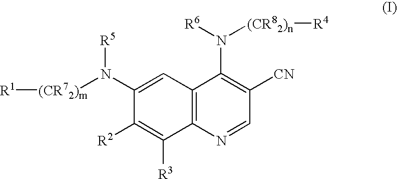
and pharmaceutically acceptable salts thereof, wherein R¹, R², R³, R⁴, R⁵, R⁶, R⁷, R⁸, m and n are defined as described herein. The invention also provides methods of making the compounds of formula (I), and methods of treating inflammatory diseases, such as rheumatoid arthritis, in a mammal comprising administering a therapeutically effective amount of a compound of formula (I) to the mammal.

### FIELD OF THE INVENTION

The invention relates to substituted 3-cyanoquinolines that are capable of modulating Tpl-2 kinase and to methods for the preparation of the substituted 3-cyanoquinolines. The cyanoquinolines of the present invention are useful for the treatment of inflammatory diseases, such as rheumatoid arthritis.

### BACKGROUND

Protein kinases are a class of enzymes that catalyze the transfer of a phosphate group from ATP to a tyrosine, serine, threonine, or histidine residue located on a protein substrate, many of which play a role in normal cell growth. Protein tyrosine kinases (PTKs) play a key role in signal transduction pathways that regulate cell division and differentiation. Certain growth factor receptor kinases have been identified as markers for a poor prognosis in many human cancers if they are overexpressed. See Hickey e al. J. Cancer, 1994, 74:1693.

Similar to PTKs, serine/threonine kinases are also involved in the regulation of cell growth. The MEK kinase Tpl-2 (also known as Cot and MAP3K8) is a serine/threonine kinase that has been shown to be a protooncogene when it is cleaved at its C-terminus. See Beinke et al., Mol. Cell Biol., 2003, 23:4739-4752.

Tpl-2 is known to be upstream in the MEK-ERK pathway and is essential for LPS induced tumor necrosis factor-α (TNF-α) production, as demonstrated by the Tpl2 knockout mouse (Tsichlis et. al. EMBO J., 1996, 15, 817). Tpl-2 is also required for TNF-α signaling (i.e. the cellular response to ligation of the TNF-α receptor). TNF-α is a pro-inflammatory cytokine that is involved in inflammation in a number of disease states, most notably in the autoimmune disease rheumatoid arthritis (RA). A protein therapeutic ENBREL/etanercept (sTNRRα) is currently available to patients with RA. However, an orally available small molecule that inhibits TNF-α synthesis and/or signaling is desirable. Tpl2 is not inhibited by staurosporine and it is the only human kinase that contains a proline instead of a conserved glycine in the glycine-rich ATP binding loop. These unique features of Tpl2 may increase the potential for discovering a selective inhibitor of the enzyme.

Heretofore, there have not been described cyanoquinolines that bind to and inhibit serine/threonine protein kinases and inhibit TNF-α synthesis and/or signaling that are useful in the treatment of inflammatory diseases. The present invention provides 4,6-diamino-3-cyanoquinolines that are inhibitors of the serine/threonine kinase Tpl-2 and can be used to treat inflammatory diseases, such as RA. This invention also provides methods of making the 4,6-diamino-3-cyanoquinolines. Not wishing to be bound by any theory, it is believed that the compounds of the present invention are useful in the treatment of inflammatory disease states, such as RA, because they have a double benefit of blocking both TNF-α production and signaling.

### SUMMARY OF THE INVENTION

The present invention provides compounds of formula (I): 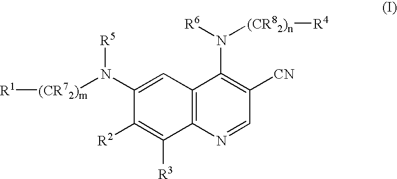
and pharmaceutically acceptable salts thereof, wherein R¹, R², R³, R⁴, R⁵, R⁶, R⁷, R⁸, m and n are defined as described herein. The invention also provides methods of making the compounds of formula (I), and methods of treating inflammatory diseases, such as rheumatoid arthritis, comprising administering a therapeutically effective amount of a compound of formula (I) to a mammal.

### DETAILED DESCRIPTION

The invention provides compounds of formula (I): 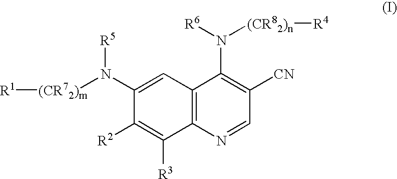
wherein:

R¹ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) N₃, e) OR⁹, f) NR¹⁰R¹¹, g) oxo, h) thioxo, i) S(O)ₚR⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl,

wherein any of o)-x) optionally is substituted with 1-4 R¹² groups;

alternatively, R¹ is selected from the group consisting of halogen, C₁₋₆ alkyl optionally substituted with 1-4 R¹² groups, C₁₋₆ haloalkyl, OR⁹, NR¹⁰R¹¹, C(O)OR⁹, C(O)NR¹⁰R¹¹, S(O)ₚR⁹, and N₃;

R² is selected from the group consisting of:
a) H, b) halogen, c) CN, d) NO₂, e) OR⁹, f) NR¹⁰OR¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₃₋₁₀ cycloalkyl, r) aryl, s) 3-10 membered cycloheteroalkyl, and t) heteroaryl,

wherein any of l)-t) optionally is substituted with 1-4 R¹² groups;

R³is selected from the group consisting of:
a) H, b) halogen, c) CN, d) NO₂, e) OR⁹, f) NR¹⁰OR¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₁₋₆ haloalkyl, r) C₃₋₁₀ cycloalkyl, s) aryl, t) 3-10 membered cycloheteroalkyl, and u) heteroaryl,

wherein any of l)-u) optionally is substituted with 1-4 R¹² groups;

R⁴is selected from the group consisting of C3-10 cycloalkyl, aryl, C3-10 cycloheteroalkyl, and heteroaryl, each optionally substituted with 1-4 moieties selected from the group consisting of:
a) halogen, b) CN, c) NO₂, d) OR⁹, e) NR¹⁰R¹¹, f) oxo, g) thioxo, h) S(O)ₚR⁹, i) SO₂NR¹⁰R¹¹, j) C(O)R⁹, k) C(O)OR⁹, l) C(O)NR¹⁰R¹¹, m) Si(C₁₋₆ alkyl)₃, n) C₁₋₆ alkyl, o) C₂₋₆ alkenyl, p) C₂₋₆ alkynyl, q) C₁₋₆ alkoxy, r) C₁₋₆ alkylthio, s) C₁₋₆ haloalkyl, t) C₃₋₁₀ cycloalkyl, u) aryl, v) 3-10 membered cycloheteroalkyl, and w) heteroaryl,
wherein any of n)-w) optionally is substituted with 1-4 R¹² groups;

alternatively, R⁴is selected from the group consisting of C₁₋₆ alkyl optionally substituted with 1-4 R¹² groups, C₁₋₆ haloalkyl, C(O)OR⁹, C(O)NR¹⁰R¹¹, S(O)ₚR⁹, and N₃;

R⁵ and R⁶ at each occurrence independently are selected from the group consisting of:
a) H, b) C(O)R⁹, c) C(O)OR⁹, d) C(O)NR¹⁰R¹¹, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl,

wherein any of e)-l) optionally is substituted with 1-4 R¹² groups;

R⁷ and R⁸ at each occurrence independently are selected from the group consisting of:
a) H, b) halogen, c) OR⁹, d) NR¹⁰R¹¹, e) C₁₋₆ alkyl, f) C₂₋₆ alkenyl, g) C₂₋₆ alkynyl, h) C₁₋₆ haloalkyl, and i) aryl;

alternatively, any two R⁷ or R⁸ groups and the carbon to which they are bonded may form a carbonyl group;

R⁹ at each occurrence is selected from the group consisting of:
a) H, b) C(O)R¹³, c) C(O)OR¹³, d) C(O)NR¹³R¹⁴, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl;

wherein any of e)-l) optionally is substituted with 1-4 R¹⁵ groups;

R¹⁰ and R¹¹ at each occurrence independently are selected from the group consisting of:
a) H, b) OR¹³, c) SO₂R¹³, d) C(O)R¹³, e) C(O)OR¹³, f) C(O)NR¹³R¹⁴, g) C₁₋₆ alkyl, h) C₂₋₆ alkenyl, i) C₂₋₆ alkynyl, k) C₁₋₆ haloalkyl, l) C₃₋₁₀ cycloalkyl, m) aryl, n) 3-10 membered cycloheteroalkyl, and o) heteroaryl;

wherein any of g)-o) optionally is substituted with 1-4 R¹⁵ groups;

R¹² at each occurrence independently is selected from the group consisting of:
a) halogen, b) CN, c) NO₂, d) N₃, e) OR⁹, f) NR¹⁰R¹¹, g) oxo, h) thioxo, i) S(O)^{p}R⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl;

wherein any of o)-x) optionally is substituted with 1-4 R¹⁵ groups;

R¹³ and R¹⁴ at each occurrence independently are selected from the group consisting of:
a) H, b) C₁₋₆ alkyl, c) C₂₋₆ alkenyl, d) C₂₋₆ alkynyl, e) C₁₋₆ haloalkyl, f) C₃₋₁₀ cycloalkyl, g) aryl, h) 3-10 membered cycloheteroalkyl, and i) heteroaryl,

wherein any of b)-j) optionally is substituted with 1-4 R¹⁵ groups;

R¹⁵ at each occurrence independently is selected from the group consisting of:
a) halogen, b) CN, c) NO₂, d) N₃, e) OH, f) O-C₁₋₆alkyl, g) NH₂, h) NH(C₁₋₆alkyl), i) N(C₁₋₆alkyl)₂, j) NH(aryl), k) NH(cycloalkyl), l) NH(heteroaryl), m) NH(cycloheteroalkyl), n) oxo, o) thioxo, p) SH, q) S(O)ₚ-C₁₋₆alkyl, r) C(O)—C₁₋₆alkyl, s) C(O)OH, t) C(O)O—C₁₋₆alkyl, u) C(O)NH₂, v) C(O)NHC₁₋₆ alkyl, w) C(O)N(C₁₋₆ alkyl)₂, x) C₁₋₆ alkyl, y) C₂₋₆ alkenyl, z) C₂₋₆ alkynyl, aa) C₁₋₆ alkoxy, bb) C₁₋₆ alkylthio, cc) C₁₋₆ haloalkyl, dd) C₃₋₁₀ cycloalkyl, ee) aryl, ff) 3-10 membered cycloheteroalkyl, and gg) heteroaryl,

wherein any C₁₋₆alkyl, C₂₋₆ alkenyl, C₂₋₆ alkynyl, C₃₋₁₀ cycloalkyl, aryl, 3-10 membered cycloheteroalkyl, or heteroaryl, alone as a part of another moiety, optionally is substituted with one or more moieties selected from the group consisting of halogen, CN, NO₂, OH, O-C₁₋₆alkyl, NH₂, NH(C₁₋₆alkyl), N(C₁₋₆alkyl)₂, NH(aryl), NH(cycloalkyl), NH(heteroaryl), NH(cycloheteroalkyl), oxo, thioxo, SH, S(O)ₚ—C₁₋₆ alkyl, C(O)—C₁₋₆alkyl, C(O)OH, C(O)O—C₁₋₆alkyl, C(O)NH₂, C(O)NHC₁₋₆ alkyl, C(O)N(C₁₋₆ alkyl)₂, C₁₋₆ alkyl, C₂₋₆ alkenyl, C₂₋₆ alkynyl, C₁₋₆ alkoxy, C₁₋₆ alkylthio, C₁₋₆ haloalkyl, C₃₋₁₀ cycloalkyl, aryl, 3-10 membered cycloheteroalkyl, and heteroaryl;

m is 0, 1, 2, 3, or 4;

n is 0 or 1; and

p is 0, 1,or 2;

or a pharmaceutically acceptable salt thereof,

provided that the compound of formula (I) does not comprise:

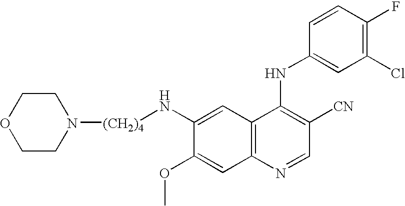

### 4-(3-Chloro-4-fluoro-phenylamino)-7-methoxy-6-(4-morpholin-4-yl-butylamino)-quinoline-3-carbonitrile

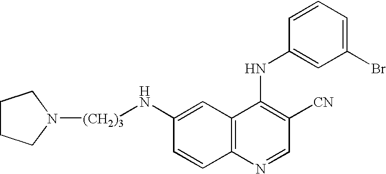

### 4-(3-Bromo-phenylamino)-6-(3-pyrrolidin-1-yl-propylamino)-quinoline-3-carbonitrile

R¹ may be a 5 or 6 membered heteroaryl, such as imidazole, triazole (e.g., 1,2,3-triazole), tetrazole, pyridine, or N-oxypyridine.

In certain embodiments, R² is H or alkylthio optionally substituted with NR¹⁰R¹¹ (e.g., SCH₂CH₂N(CH₃)₂).

In some embodiments, R³ is H or a halogen, such as Cl or Br.

R⁴ may be phenyl optionally substituted with 1-2 halogens, such as Cl or F. In some embodiments, R⁴ is phenyl substituted with Cl and F, such as 3-chloro-4-fluorophenyl.

R⁵ may be, for instance, H or C₁₋₆ alkyl.

Examples of R⁶ include H and C₁₋₆ alkyl.

In certain embodiments, m is 1.

In some embodiments, n is 0.

In some embodiments, when m is 2, 3, or 4, R¹ is not morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran or 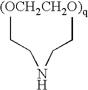
wherein q is 1-4.

In other embodiments, when R¹ is a saturated 3-8 membered cycloheteroalkyl, R¹ is not substituted with —(CR⁸₂)ᵣ-Het1 or —(CR⁸₂)ₛ—Y—(CR⁸₂)ₜ-Het1, wherein

Het1 is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, pyrrole, and tetrahydropyran;

Y is selected from the group consisting of O, S, NR¹⁰C(O), C(O)NR¹⁰, and NR¹⁰;

r is 0-8;

s is 0-4; and

t is 0-4.

The invention also includes intermediates of the compounds described herein having the formula (II): 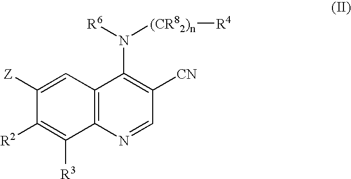
wherein Z is halogen, C₁₋₆ alkyl optionally substituted with 1-4 R¹² groups, C₁₋₆ haloalkyl, OR⁹, NR¹⁰R¹¹, S(O)ₚR⁹, SO₂NR¹⁰R¹¹, C(O)R⁹, C(O)OR⁹, C(O)NR¹⁰R¹¹, or N₃, and R², R³, R⁴, R⁶, R⁸, R⁹, R¹⁰, R¹¹ and n are defined as described above.

The invention also includes pharmaceutical compositions that include one or more compounds according to the invention, or pharmaceutically salts thereof, and one or more pharmaceutically acceptable carriers.

The compounds of the present invention are useful for the treatment of disease conditions mediated by Tpl2, such as rheumatoid arthritis (RA), juvenile RA, psoriatic arthritis, ankylosing spondylitis, and osteoarthritis and for the alleviation of symptoms thereof. Accordingly, the present invention further provides methods of treating these diseases and disorders using the compounds described herein. In some embodiments, the methods include identifying a mammal having a disease or disorder mediated by Tpl2, and providing to the mammal an effective amount of a compound as described herein.

In further embodiments, the methods are provided for alleviating a symptom of a disease or disorder mediated by Tpl2. In some embodiments, the methods include identifying a mammal having a symptom of a disease or disorder mediated by Tpl2, and providing to the mammal an amount of a compound as described herein effective to ameliorate (i.e., lessen the severity of) the symptom.

Pharmaceutically acceptable salts of the compounds of Formula (I) having an acidic moiety can be formed from organic and inorganic bases. Suitable salts with bases are, for example, metal salts, such as alkali metal or alkaline earth metal salts, for example sodium, potassium, or magnesium salts; or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Similarly, when a compound of the present invention contains a basic moiety, salts can be formed from organic and inorganic acids. For example, salts can be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, or camphorsulfonic acid, or other known pharmaceutically acceptable acids.

The present invention also includes prodrugs of the compounds described herein. As used herein, “prodrug” refers to a moiety that releases a compound of the invention when administered to a mammalian subject. Prodrugs can be prepared by modifying functional groups present in the compounds in such a way that the modifications are cleaved, either by routine manipulation or in vivo, to the parent compounds. Examples of prodrugs include compounds of the invention as described herein that contain one or more molecular moieties appended to a hydroxyl, amino, sulfhydryl, or carboxyl group of the compound, and that when administered to a mammalian subject, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

The present invention provides pharmaceutical compositions comprising at least one compound according to the invention and one or more pharmaceutically acceptable carriers, excipients, or diluents. Examples of such carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable. Supplementary active ingredients can also be incorporated into the compositions.

The compounds of the invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antiinflammatory agents. Oral formulations containing the active compounds of this invention may comprise any conventionally used oral form, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets may contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Examples of liquid carriers for oral and parenteral administration include water (particularly containing additives as described above, e.g. cellulose derivatives, such as a sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally, i.e., administered across the surface of the body and the inner linings of bodily passages including epithelial and mucosal tissues. Such administrations may be carried out using the present compounds or pharmaceutically acceptable salts thereof, in lotions, creams, foams, patches, suspensions, solutions, and suppositories (rectal and vaginal). Topical formaulations that deliver the compounds of the invention through the epidermis may be useful for localized treatment of inflammation and arthritis.

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

Lipid formulations or nanocapsules may be used to introduce the compounds of the present invention into host cells either in vitro or in vivo. Lipid formulations and nanocapsules may be prepared by methods known in the art.

In order to increase the effectiveness of the compounds of the present invention, it may be desirable to combine these compositions with other agents effective in the treatment of the target disease. For inflammatory diseases, other agents effective in their treatment, and particularly in the treatment of rheumatoid arthritis, may be administered with the compounds of the present invention. For cancer, additional anti-cancer agents may be administered. The other agents may be administered at the same time or at different times than the compounds of the present invention.

As used herein, “halo” or “halogen” includes fluoro, chloro, bromo, and iodo.

As used herein, “oxo” refers to a double-bonded oxygen (i.e., ═O).

As used herein, the term “alkyl” refers to a straight-chain or branched saturated hydrocarbon group. Alkyl groups can contain from 1 to about 20, 1 to about 10, 1 to about 8, 1 to about 6, 1 to about 4, or 1 to about 3 carbon atoms. Alkyl groups preferably contain 1 to 6 carbon atoms. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can be substituted with up to four independently selected R¹² groups, as described herein.

As used herein, “alkenyl” refers to a straight-chain or branched alkyl group as defined above having one or more double carbon-carbon bonds. Alkenyl groups preferably contain 2 to 6 carbon atoms. Examples of alkenyl groups include ethenyl, propenyl, butenyl, pentenyl, hexenyl, butadienyl, pentadienyl, hexadienyl, and the like. Alkenyl groups can be substituted with up to four independently selected R¹² groups, as described herein.

As used herein, “alkynyl” refers to a straight-chain or branched alkyl group as defined above having one or more triple carbon-carbon bonds. Alkynyl groups preferably contain 2 to 6 carbon atoms. Examples of alkynyl groups include ethynyl, propynyl, butynyl, pentynyl, and the like. Alkynyl groups can be substituted with up to four independently selected R¹² groups, as described herein.

As used herein, “alkoxy” refers to an —O-alkyl group, wherein alkyl is as defined above. Alkoxy groups preferably contain 1 to 6 carbon atoms. Examples of alkoxy groups include methoxy, ethoxy, propoxy (e.g., n-propoxy and isopropoxy), t-butoxy, and the like. Alkoxy groups can be substituted with up to four independently selected R¹² groups, as described herein.

As used herein, “alkylthio” refers to an —S-alkyl group, wherein alkyl is as defined above. Alkylthio groups preferably contain 1 to 6 carbon atoms. Alkylthio groups can be substituted with up to four independently selected R¹² groups, as described herein.

As used herein, “haloalkyl” refers to an alkyl group, as defined above, having one or more halogen substituents. Haloalkyl groups preferably contain 1 to 6 carbon atoms. Examples of haloalkyl groups include CF₃, C₂F₅, CHF₂, CCl₃, CHCl₂, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to non-aromatic carbocyclic groups including cyclized alkyl, alkenyl, and alkynyl groups. Cycloalkyl groups can be monocyclic (e.g., cyclohexyl) or poly-cyclic (e.g. fused, bridged, or spiro ring systems), wherein the carbon atoms are located inside or outside of the ring system. Cycloalkyl groups preferably contain 3 to 10 carbon atoms. Any suitable ring position of the cycloalkyl moiety may be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcarnyl, adamantyl, spiro[4.5]deanyl, homologs, isomers, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like. Cycloalkyl groups can be substituted with up to four independently selected R¹² groups, as described herein.

As used herein, “aryl” refers to C₆₋₂₀ aromatic monocyclic or polycyclic hydrocarbons such as, for example, phenyl, 1 -naphthyl, 2-naphthyl anthracenyl, phenanthrenyl, and the like. Any suitable ring position of the aryl moiety may be covalently linked to the defined chemical structure. Aryl groups can be substituted with up to four independently selected R¹² groups, as described herein.

As used herein, “heteroaryl” refers to monocyclic or polycyclic aromatic ring systems having from 5 to 20 ring atoms and containing 1-3 ring heteroatoms selected from oxygen (O), nitrogen (N) and sulfur (S). Generally, heteroaryl rings do not contain O—O, S—S, or S—O bonds. Heteroaryl groups include monocyclic heteroaryl rings fused to a phenyl ring. The heteroaryl group may be attached to the defined chemical structure at any heteroatom or carbon atom that results in a stable structure. Examples of heteroaryl groups include, for example: 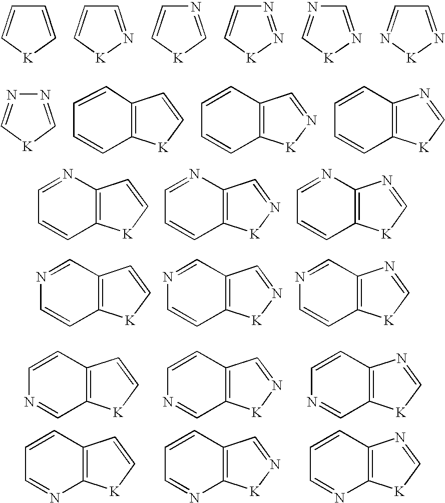
wherein K is defined as O, S, N or NR¹⁰. One or more N or S in a heteroaryl ring may be oxidized (e.g., pyridine N-oxide). Examples of heteroaryl rings include pyrrole, furan, thiophene, pyridine, pyrimidine, pyridazine, pyrazine, triazole, pyrazole, imidazole, isothiazole, thiazole, isoxazole, oxazole, indole, isoindole, benzofuran, benzothiophene, quinoline, isoquinoline, quinoxaline, quinazoline, benzotriazole, indazole, benzimidazole, benzothiazole, benzisoxazole, 2-methylquinoline-4-yl, 1-H-1,2,3-benzotriazol-1-yl, 1-H-benzimidazol-5-yl, 2,1,3-benzoxadiazol-5-yl, benzoxazole, benzimidazolyl, benzofuranyl, benzothiofuranyl, benzothiophenyl, benzoxazolyl, benzthiazolyl, benztriazolyl, benztetrazolyl, benzo[c]isoxazolyl, benzo[d]isoxazolyl, benzo[c]isothiazolyl, benzo[d]isothiazolyl, cinnolinyl, 1H-indazolyl, 2H-indazolyl, indolizinyl, indolyl, isobenzofuranyl, isoindolyl, isoquinolinyl, naphthyridinyl, phthalazinyl, pteridinyl, purinyl, oxazolopyridinyl, thiazolopyridinyl, imidazopyridinyl, furopyridinyl, thienopyridinyl, pyridopyrimidine, pyridopyrazine, pyridopyridazine, quinazolinyl, quinolinyl, quinoxalinyl, thienothiazolyl, thienoxazolyl, and thienoimidazolyl. Heteroaryl groups can be substituted with up to four independently selected R¹² groups as described herein.

As used herein, “cycloheteroalkyl” refers to a non-aromatic cycloalkyl group that contains at least one ring heteroatom selected from O, N and S, and optionally contains one or more double or triple bonds. One or more N or S in a cycloheteroalkyl ring may be oxidized (e.g., thiomorpholine S-oxide, thiomorpholine S,S-dioxide). Cycloheteroalkyl groups preferably contain 3 to 10 ring atoms, 1-3 of which are heteroatoms selected from O, S, and N. Examples of cycloheteroalkyl groups include morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine piperazine, and the like. Cycloheteroalkyl groups can be optionally substituted with up to four independently selected R¹² groups as described herein. Nitrogen atoms of cycloheteroalkyl groups can bear a substituent, for example an R⁵ group, as described herein. Also included in the definition of cycloheteroalkyl are moieties that have one or more aromatic rings fused (i.e., having a bond in common with) to the cycloheteroalkyl ring, for example, benzimidazolinyl, chromanyl, chromenyl, indolinetetrahydorquinolinyl, and the like. Cycloheteroalkyl groups can also contain one or more oxo groups, such as phthalimide, piperidone, oxazolidinone, pyrimidine-2,4(1H,3H)-dione, and pyridin-2(1H)-one, and the like.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The compounds of the present invention can contain an asymmetric atom (also referred as a chiral center), and some of the compounds can contain one or more asymmetric atoms or centers, which can thus give rise to optical isomers (enantiomers) and diastereomers. The present invention includes such optical isomers (enantiomers) and diastereomers (geometric isomers); as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. The present invention also encompasses cis and trans isomers of compounds containing alkenyl moieties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of present invention can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Compounds of the invention may be synthesized, for example, according to Scheme I below.

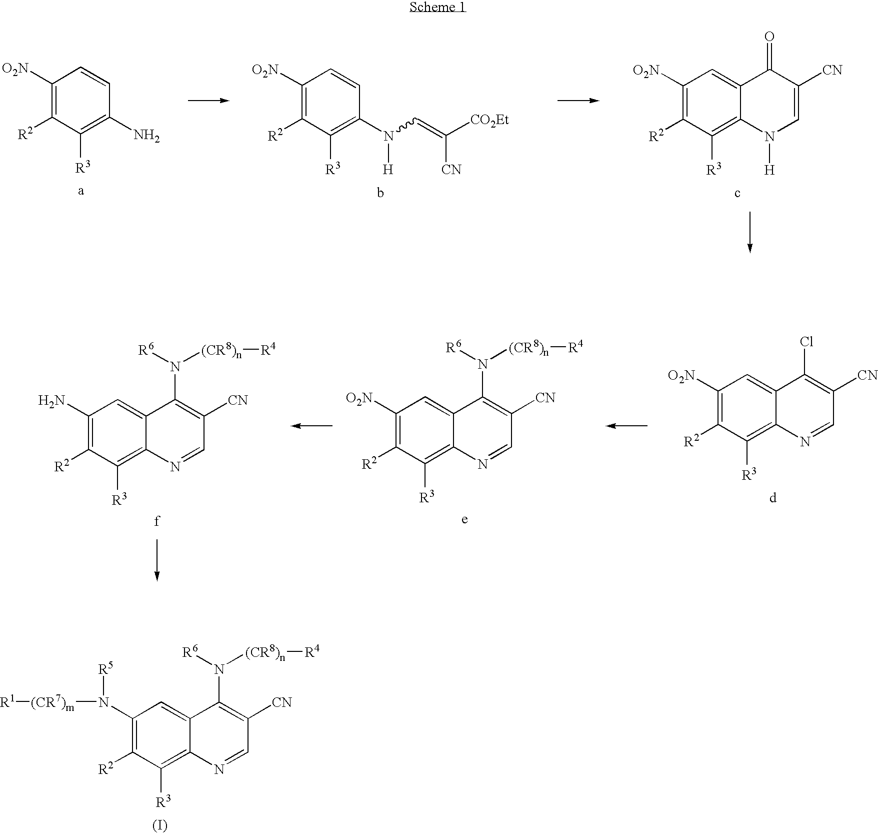

As shown in Scheme I, an unsubstituted or substituted 4-nitroaniline derivative a is reacted with ethoxy ethylenecyanoacetate, preferably in a solvent such as benzene, toluene or DMF to give a cyano-3-4-nitrophenylaminoacrylic acid ethyl ester intermediate b. The intermediate b is heated in a solvent such as Dowtherm A (Dow Chemical Company, Midland, Mich.) to give a quinolone c. The quinolone c is converted to a chlorocyanoquinoline d by heating with a chlorinating agent such as POCl₃ or SOCl₂ either as a neat solution or in a solvent such as toluene. The chlorocyanoquinoline d is heated with an amine having the formula HNR⁶(CR⁸₂)ₙR⁴ to give the intermediate e. The nitro group of the intermediate e can be reduced to the amine using a reducing agent (e.g., tin (II) chloride dihydrate or ferrous chloride and ammonium chloride) to provide the 6-amino intermediate f. The intermediate f can be alkylated by treatment with an aldehyde or ketone (e.g., R¹(CR⁷₂)ₘC(O)H or R¹(CR⁷₂)ₘC(O)(CR⁷₂)₄₋ₘ) and a reducing agent (e.g., sodium cyanoborohydride or sodium triacetoxyborohydride) to give a 4,6-diamino-3-cyanoquinoline of formula (I). Alternatively, intermediate f may be alkylated, for example, with a compound having the formula R¹(CR⁷₂)ₘX (wherein X is a suitable leaving group, e.g., Cl, Br, mesylate, tosylate, etc.) in the presence of a base to give a 4,6-diamino-3-cyanoquinoline of formula (I). The C-6 amine may be further functionalized to add an R⁵ group.

Functionalization at the C-7 and/or C-8 positions of the quinoline ring may be carried out prior to the formation of intermediate b. For example, 4-nitroaniline may be treated with a brominating agent (e.g., Br₂ in acetic acid) to form 2-bromo-4-nitroaniline, which can then be used to synthesize compounds of formula (I) wherein R³ is Br according to Scheme I above. Further functionalization at C-7 and/or C-8 may be carried out, for example, by treating compounds of formula (I) wherein R² and/or R³ is a halogen with an organozinc, organotin, organoboronic acid or organocopper reagent and a catalyst (e.g.,palladium (bistriphenylphosphine) dichloride) to give C-7 and/or C-8 subsituted 3-cyanoquinolines.

Scheme II depicts another exemplary method for synthesizing compounds of the invention.

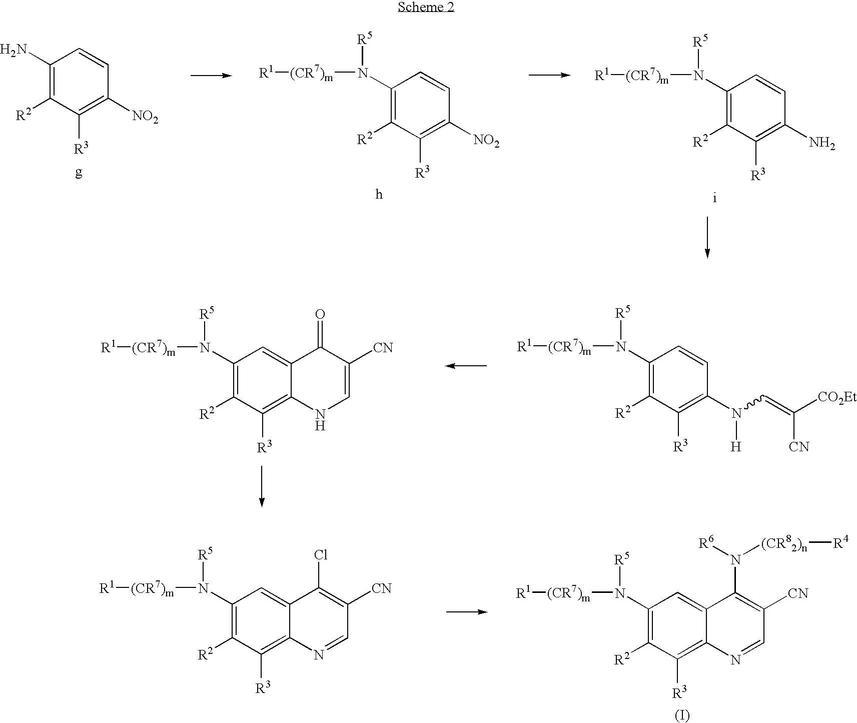

According to Scheme II, an unsubstituted or substituted 4-nitroaniline derivative g is alkylated (e.g., using the reductive amination or alkylation conditions described above) to form the alkylated intermediate h. The nitro group of intermediate h is then reduced to the amine to form diamine intermediate i, which can then be converted to the 4,6-diamino-3-cyanoquinolines of formula (I) according to the procedures described in Scheme I above.

### EXAMPLES

The following describes the preparation of representative compounds of this invention in greater detail. The following examples are offered for illustrative purposes, and are not intended to limit the invention in any manner. Those of skill in the art will readily recognize a variety of parameters that can be changed or modified to yield essentially the same results.

Mass spectral data is reported as the mass-to-charge ratio, m/z; and for high resolution mass spectral data, the calculated and experimentally found masses, [M+H]⁺, for the neutral formulae M are reported. Nuclear magnetic resonance data is reported as δ in parts per million (ppm) downfield from the standard (tetramethylsilane), along with the solvent, nucleus, and field strength parameters. The spin-spin homonuclear coupling constants are reported as J values in hertz; and the multiplicities are reported as a: s, singlet; d, doublet; t, triplet; q, quartet; quintet; or br, broadened.

### Example 1

### N-[4-(3-Chloro-4-fluoro-phenylamino)-3-cyano-quinolin-6-yl]-benzamide

Step 1: 6-bromo-4-chloro-quinoline-3-carbonitrile (2.5 g, 9.4 mmol) was taken up in 2-ethoxyethanol (110 mL) and 3-chloro-4-fluoroniline (1.43 g, 9.8 mmol) was added and heated at reflux (135° C.) for 2.5 hours or until complete by TLC. The reaction was cooled to room temperature and solid precipitated out. The solution was filtered to obtain 6-bromo-4-(3-chloro-4-fluoro-phenylamino)-quinoline-3-carbonitrile. Yield: 56%: 1H NMR (400 MHz, DMSO-D6) δ ppm 7.47 (dq, J=6.63, 4.36, 2.53, 2.15 Hz, 1 H) 7.56 (t, J=8.97 Hz, 1 H) 7.75 (dd, J=6.69, 2.65 Hz, 1 H) 7.98 (d, J=8.84 Hz, 1 H) 8.16 (dd, J=8.97, 1.89 Hz, 1 H) 8.97 (s, 1 H) 9.01 (d, J=2.02 Hz, 1 H) 11.08 (s, 1 H).

Step 2: A mixture of 6-bromo-4- (3-chloro-4-fluoro-phenylamino)-quinoline-3-carbonitrile (100 mg, 0.27 mmol), benzamide (77 mg, 0.64 mmol), K₃PO₄ (113 mg, 0.53 mmol), Cul (20 mg, 20 wt % eq), and trans-1,2-diaminocyclohexane (20 uL, 20 wt % eq) was suspended in 4mL of dioxane, flushed with N₂ and heated at 150° C. for 1 hour in the microwave. After the desired product formation was confirmed by LC/MS, the solution was filtered and solvent removed. The resulting crude material was purified via prep-HPLC to give N-[4-(3-Chloro-4-fluoro-phenylamino)-3-cyano-quinolin-6-yl]-benzamide (37 mg, 33% yield): 1H NMR (400 MHz, DMSO-D6) δ ppm 7.42 (none, 1 H) 7.49 (s, 1 H) 7.54-7.66 (m, 3 H) 8.00-8.05 (m, 2 H) 8.08 (dd, J=8.97, 2.40 Hz, 1 H) 8.56 (s, 1 H) 8.92 (s, 1 H) 10.65 (s, 1 H).

### Example 2

### N-[4-(3-Chloro-4-fluoro-phenylamino)-3-cyano-quinolin-6-yl]-nicotinamide

Coupling of 6-Bromo-4-(3-chloro-4-fluoro-phenylamino)-quinoline-3-carbonitrile (100 mg, 0.27 mmol) with nicotinamide (78 mg, 0.64 mmol) was carried out according to Example 1, step 2, to obtain N-[4-(3-Chloro-4-fluoro-phenylamino)-3-cyano-quinolin-6-yl]-nicotinamide (39 mg, 35% yield): 1H NMR (400 MHz, DMSO-D6) δ ppm 7.29 (s, 1 H) 7.44 (t, J=9.09 Hz, 2 H) 7.51 (d, J=6.57 Hz, 1 H) 7.57-7.66 (m, 1 H) 7.98 (s, 1 H) 8.03-8.12 (m, 1 H) 8.36 (d, J=8.59 Hz, 1 H) 8.59 (s, 1 H) 8.80 (d, J=4.55 Hz, 1 H) 8.91 (s, 1 H) 9.17 (s, 1 H) 9.86 (s, 2 H) 10.84 (s, 1 H); HRMS (ESI+) calcd for C₂₂H₁₃ClFN₅O (MH+) 418.08654, found 418.0869.

### Example 3

### 4-(3-Chloro-4-fluoro-phenylamino)-6-[(furan-2-ylmethyl)-amino]-quinoline-3-carbonitrile

Step 1: 2-Cyano-3-(4-nitro-phenylamino)-acrylic acid ethyl ester (25 g, 95.8 mmol) was suspended in Dowtherm A (1 L) and heated at 260° C. for 18 hours. The reaction was cooled to room temperature (RT), then poured into 1.5 L of hexanes and stirred for 1 hour. The dark brown solid was collected via suction filtration, triturated in refluxing ethanol (200 mL) for 15 min then cooled to RT and stirred for 12 hours. The solid was collected by suction filtration to obtain 6-nitro-4-oxo-1,4-dihydro-quinoline-3-carbonitrile (1 5.4 g) in 75% yield: 1H NMR (400 MHz, DMSO-D6) δ ppm 7.82 (d, J=9.09 Hz, 1 H) 8.53 (dd, J=9.09, 2.53 Hz, 1 H) 8.81 (d, J=2.53 Hz, 1 H) 8.90 (s, 1 H) 13.27 (s, 1 H).

Step 2: The product from Step 1 (6 g, 27.9 mmol) was suspended in POCI₃ (45 mL) and heated at reflux for 6 hours then cooled to RT. The solution became very thick and was slurried with ethyl acetate and stripped to dryness. Residue was scraped out and poured over ice. As the ice melted, the pH was adjusted to ˜8 using solid NaHCO₃. The solid was collected via suction filtration, washed with water and hexanes and dried under high vacuum for 24 hours to obtain 4-Chloro-6-nitro-quinoline-3-carbonitrile (6.15 g) in 95% yield: 1H NMR (400 MHz, DMSO-D6) δ ppm 8.43 (d, J=9.35 Hz, 1 H) 8.72 (dd, J=9.09, 2.53 Hz, 1 H) 9.06 (d, J=2.53 Hz, 1 H) 9.43 (s, 1 H).

Step 3: The product from Step 2 (2.33 g, 10 mmol) and 3-chloro-4-fluoroaniline (1.74 g, 12 mmol) were suspended in ethanol (60 mL) and heated at reflux for 3 hours or until completed by TLC. After cooling, the solvent was removed in vacuum and the residue was triturated in ether/sat'd aqueous NaHCO₃ (100 mL/75 mL) for 2.5 hours. The solid was collected by suction filtration and dried under high vacuum for 24 hours to obtain 4-(3-chloro-4-fluoro-phenylamino)-6-nitro-quinoline-3-carbonitrile (2.75 g) in 80% yield: 1H NMR (400 MHz, DMSO-D6) δ ppm 7.28-7.35 (m, 1 H) 7.47 (t, J=8.97 Hz, 1 H) 7.56 (dd, J=6.69, 2.65 Hz, 1 H) 7.99 (d, J=9.09 Hz, 1 H) 8.49 (dd, J=9.35, 2.53 Hz, 1 H) 8.64 (s, 1 H) 9.47 (d, J=2.27 Hz, 1 H) 10.72 (s, 1 H).

Step 4: The product from Step 3 (2.5 g, 7.29 mmol) was suspended in ethanol (85 mL), then tin chloride dihydrate (8.3 g, 36.5 mmol) was added and the reaction was heated at reflux for 2.5 hours or until complete by TLC. The reaction was diluted with 100 mL of water, and then solid NaHCO₃ was added until the pH was basic (˜11 g). The solution was extracted with chloroform, washed with brine, treated with activated carbon, dried over Mg₂SO₄, and stripped to obtain 6-amino-4-(3-chloro-4-fluoro-phenylamino)-quinoline-3-carbonitrile (2.04 g) in 90% yield: 1H NMR (400 MHz, DMSO-D6) δ ppm 5.78 (s, 2 H) 7.12-7.19 (m, 2 H) 7.25 (dd, J=8.84, 2.27 Hz, 1 H) 7.34-7.42 (m, 2 H) 7.70 (d, J=9.09 Hz, 1 H) 8.34 (s, 1 H) 9.36 (s, 1 H).

Step 5: The product from Step 4 (150 mg, 0.48 mmol) and 2-furaldehyde (95 uL, 1.15 mmol) were taken up in ethanol (8 mL), then acetic acid (700 uL) and NaCNBH₃ (36 mg, 0.58 mmol) were added and the reaction warmed at 30° C. for 2.5 hours or until completed by TLC. The reaction was stripped to dryness and the residue was purified by flash chromatography eluting with 0-10% MeOH in CH₂Cl₂ to obtain 4-(3-Chloro-4-fluoro-phenylamino)-6-[(furan-2-ylmethyl)-amino]-quinoline-3-carbonitrile (1 93 mg) in 95 % yield: 1H NMR (400 MHz, DMSO-D6) δ ppm 6.35 (dd, J=3.28, 0.76 Hz, 1 H) 6.39 (dd, J=3.28,1.77 Hz, 1 H) 6.79 (t, J=5.56 Hz, 1 H) 7.21-7.27 (m, 3 H) 7.35 (dd, J=9.09, 2.53 Hz, 1 H) 7.43 (t, J=8.97 Hz, 1 H) 7.48 (dd, J=6.69, 2.65 Hz, 1 H) 7.60 (dd, J=1.77, 0.76 Hz, 1 H) 7.70 (d, J=9.09 Hz, 1 H) 8.33 (s, 1 H); HRMS (ESI+) calcd for C₂₁H₁₄ClFN₄O (MH+) 393.09129, found 393.0917.

### Example 4

### 4-(3-Chloro-4-fluoro-phenylamino)-6-[(3H-imidazol-4-ylmethyl)-amino]-quinoline-3-carbonitrile

In a 50 mL round-bottomed flask 6-amino-4-(3-chloro-4-fluoro-phenylamino)-quinoline-3-carbonitrile (0.2 g, 0.64 mmol), ethanol (10 mL) and 4(5)-imidazole carboxaldehyde (0.1 47 g, 1.53 mmol) were added. Then, acetic acid was added to bring the pH of the solution to 4, and the mixture was stirred for 15 minutes. NaCNBH₃ (48 mg, 0.77 mmol) was then added and the reaction warmed at 30° C. for 2.5 h or until complete by TLC. The reaction was stripped to dryness and the residue was purified via preparative HPLC, and lyophilized to give the product as a yellow solid (0.166 g, 66%): ¹H NMR (400 MHz, DMSO-D6) δ ppm 4.26 (d, J=4.80 Hz, 2 H) 6.53 (t, J=5.43 Hz, 1 H) 7.05 (s, 1 H) 7.20 (d, J=2.53 Hz, 1 H) 7.22-7.28 (m, 1 H) 7.38 (dd, J=8.97, 2.40 Hz, 1 H) 7.43 (t, J=9.09 Hz, 1 H) 7.48 (dd, J=6.57, 2.78 Hz, 1 H) 7.62-7.70 (m, 2 H) 8.15 (s, 2 H) 9.36 (s, 1 H); HRMS (ESI+) calcd for C₂₀H₁₄ClFN₆ (MH+) 393.10252, found 393.1019.

### Example 5

### 4-(3-Chloro-4-fluoro-phenylamino)-6-[(furan-3-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 048 mmol) was reacted with 3-furaldehyde (95 uL, 1.15 mmol) and NaCNBH₃ (36 mg, 0.58 mmol) in 8 mL EtOH. The crude product was purified by preparative HPLC, and lyophilized to give the product as a solid (1 58 mg, 85%): 1H NMR (400 MHz, DMSO-D6) δ ppm 4.20 (d, J=5.31 Hz, 2 H) 6.53 (dd, J=1.77, 0.76 Hz, 1 H) 6.61 (t, J=5.68 Hz, 1 H) 7.18 (d, J=2.27 Hz, 1 H) 7.22-7.27 (m, 1 H) 7.34 (dd, J=9.09, 2.53 Hz, 1 H) 7.43 (t, J=8.97 Hz, 1 H) 7.47 (dd, J=6.57, 2.53 Hz, 1 H) 7.63 (t, J=1.64 Hz, 1 H) 7.66-7.71 (m, 2 H) 8.30-8.34 (m, 1 H) 9.34 (s, 1 H); HRMS (ESI+) calcd for C₂₁H₁₄CIFN₄O (MH+) 393.09129, found 393.0915.

### Example 6

### 4-(3-Chloro-4-fluoro-phenylamino)-6-(3-nitro-benzylamino)-quinoline-3-carbonitrile

Following the procedure described above in Example 4, 6-amino-4-(3-chloro-4-fluoro-phenylamino)-quinoline-3-carbonitrile (300 mg, 0.96 mmol) was reacted with 3-nitrobenzaldehyde (348 mg, 2.3 mmol) and NaCNBH₃ (73 mg, 1.15 mmol) in 16 mL EtOH. The crude product was purified by preparative HPLC, and lyophilized to give the product as a solid (275 mg, 64%): 1H NMR (400 MHz, DMSO-D6) δ ppm 4.55 (d, J=6.32 Hz, 2 H) 7.07-7.12 (m, 2 H) 7.13-7.19 (m, 1 H) 7.33-7.40 (m, 3 H) 7.62 (t, J=7.96 Hz, 1 H) 7.73 (d, J=9.09 Hz, 1 H) 7.81 (d, J=8.34 Hz, 1 H) 8.08-8.13 (m, 1 H) 8.23-8.26 (m, 1 H) 8.34 (s, 1 H) 9.28 (s, 1 H); HRMS (ESI+) calcd for C₂₃H₁₅ClFN₅O₂ (MH+) 448.09711, found 448.0973.

### Example 7

### 4-(3-Chloro-4-fluoro-phenylamino)-6-[(1-methyl-1H-benzoimidazol-2-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 1-methyl-2-formylbenzimidazole (184 mg, 1.15 mmol) and NaCNBH₃ (36 mg, 0.58 mmol) in 8 mL EtOH. The crude product was purified by preparative HPLC, and lyophilized to give the product as a solid (17 mg, 8%): 1H NMR (400 MHz, acetic acid-D4) δ ppm 3.96 (s, 3 H) 5.18 (s, 2 H) 7.15-7.25 (m, 2 H) 7.37-7.49 (m, 3 H) 7.52-7.67 (m, 4 H) 7.92 (d, J=10.11 Hz, 1 H) 8.47 (s, 1 H); HRMS (ESI+) calcd for C₃₂H₃₃N₃O₅ (MH+) 540.24930, found 540.2501.

### Example 8

### 4-(3-Chloro-4-fluoro-phenylamino)-6-[(thiazol-2-ylmethyl)-amino]-quinoline-3-carbonitrile

6-amino-4-(3-chloro-4-fluoro-phenylamino)-quinoline-3-carbonitrile (150 mg, 0.48 mmol, prepared according to Example 3 above), and 2-thiazolecarboxaldehyde (84 uL, 0.96 mmol) were taken up in dioxane (8 mL) and heated at reflux for 12 hours. The reaction was cooled to RT, NaCNBH₃ (90 mg, 1.44 mmol) in methanol (3 mL) was added, and the mixture was stirred at RT for 4 hours. The reaction was stripped to dryness and residue was purified via flash chromatography eluting with 0-10% MeOH in CH₂Cl₂ to obtain 4-(3-chloro-4-fluoro-phenylamino)-6-[(thiazol-2-ylmethyl)-amino]-quinoline-3-carbonitrile (86 mg) in 45% yield: 1H NMR (400 MHz, DMSO-D6) δ ppm 4.77 (d, J=6.32 Hz, 2 H) 7.18-7.24 (m, 2 H) 7.26 (d, J=2.53 Hz, 1 H) 7.35-7.47 (m, 3 H) 7.58 (d, J=3.28 Hz, 1 H) 7.72-7.77 (m, 2 H) 8.34 (s, 1 H) 9.35 (s, 1 H); HRMS: calcd for C₂₀H₁₃ClFN₅S+H+, 410.06370; found (ESI-FTMS, [M+H]¹⁺), 410.0646.

### Example 9

### 4-(3-Chloro-4-fluoro-phenylamino)-6-[(5-hydroxymethyl-furan-2-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 5-(hydroxymethyl)furfural (145 mg, 1.15 mmol) and NaCNBH₃ (36 mg, 0.58 mmol) in 8 mL EtOH. The crude product was purified by preparative HPLC, and lyophilized to give the product as a solid (183 mg, 90%): 1H NMR (400 MHz, DMSO-D6) δ ppm 4.33-4.39 (m, 4 H) 5.76 (s, 1 H) 6.23 (dd, J=36.38, 3.03 Hz, 2 H) 6.76 (t, J=5.81 Hz, 1 H) 7.20-7.25 (m, 1 H) 7.29-7.36 (m, 2 H) 7.40 (t, J=9.09 Hz, 1 H) 7.45 (dd, J=6.57, 2.53 Hz, 1 H) 7.67 (d, J=8.84 Hz, 1 H) 8.28 (s, 1 H); HRMS (ESI+) calcd for C₂₂H₁₆ClFN₄O₂ (MH+) 423.10186, found 423.1021.

### Example 10

### 4-(3-Chloro-4-fluoro-phenylamino)-6-(3-cyano-benzylamino)-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 3-cyanobenzaldehyde (125 mg, 0.96 mmol) and NaCNBH₃ (36 mg, 0.58 mmol) in 8 mL EtOH. The crude product was purified by preparative HPLC, and lyophilized to give the product as a solid (100 mg, 49%): 1H NMR (400 MHz, DMSO-D6) δ ppm 4.45 (d, J=5.81 Hz, 2 H) 6.97 (t, J=6.19 Hz, 1 H) 7.10 (d, J=1.52 Hz, 1 H) 7.15-7.22 (m, 1 H) 7.33-7.43 (m, 3 H) 7.54 (t, J=7.71 Hz, 1 H) 7.71 (t, J=8.34 Hz, 3 H) 7.81 (s, 1 H) 8.34 (s, 1 H) 9.30 (s, 1 H); HRMS (ESI+) calcd for C₂₄H₁₅ClFN₅ (MH+) 428.10728, found 428.1077.

### Example 11

### 4-(3-Chloro-4-fluoro-phenylamino)-6-[(5-nitro-furan-2-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 5-nitro-2-furaldehyde (162 mg, 1.15 mmol) and NaCNBH₃ (36 mg, 0.58 mmol) in 8 mL EtOH. The crude product was purified by preparative HPLC, and lyophilized to give the product as a solid (81 mg, 39%): 1H NMR (400 MHz, DMSO-D6) δ ppm 4.57 (d, J=6.06 Hz, 2 H) 6.72 (d, J=3.79 Hz, 1 H) 7.02-7.08 (m, 1 H) 7.20-7.28 (m, 2 H) 7.37 (dd, J=9.09, 2.53 Hz, 1 H) 7.42 (t, J=8.97 Hz, 1 H) 7.48 (dd, J=6.57, 2.78 Hz, 1 H) 7.63 (d, J=3.79 Hz, 1 H) 7.74 (d, J=8.84 Hz, 1 H) 8.34 (s, 1 H) 9.32 (s, 1 H); HRMS (ESI+) calcd for C₂₁H₁₃ClFN₅O₃ (MH+) 438.07637, found 438.0763.

### Example 12

### 4-(3-Chloro-4-fluoro-phenylamino)-6-(4-imidazol-1-yl-benzylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 4-(1H-imidazol-1-yl)benzaldehyde (263 mg, 1.53 mmol) and NaCNBH₃ (48 mg, 0.76 mmol) in 10 mL EtOH. The crude product was purified by preparative HPLC, and lyophilized to give the product as a solid (196 mg, 66%): 1H NMR (400 MHz, DMSO-D6) δ ppm 4.43 (d, J=6.06 Hz, 2 H) 6.93 (t, J=5.68 Hz, 1 H) 7.09 (s, 1 H) 7.15 (d, J=2.02 Hz, 1 H) 7.18-7.23 (m, 1 H) 7.34-7.44 (m, 3 H) 7.50 (d, J=8.59 Hz, 2 H) 7.58-7.63 (m, 2 H) 7.69-7.74 (m, 2 H) 8.22 (s, 1 H) 8.33 (s, 1 H) 9.31 (s, 1 H); HRMS (ESI+) calcd for C₂₆H₁₈CIFN₆ (MH+) 469.13382, found 469.1327.

### Example 13

### 4-(3-Chloro-4-fluoro-phenylamino)-6-[(1H-imidazol-2-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 2-imidazole carboxaldehyde (110 mg, 1.15 mmol) and NaCNBH₃ (36 mg, 0.58 mmol) in 10 mL EtOH. The crude product was purified by preparative HPLC, and lyophilized to give the product as a solid (103 mg, 55%): 1H NMR (400 MHz, DMSO-D6) δ ppm 4.38 (d, J=5.31 Hz, 2 H) 6.69 (t, J=5.05 Hz, 1 H) 6.96 (s, 2 H) 7.21-7.29 (m, 2 H) 7.36-7.51 (m, 3 H) 7.70 (d, J=9.35 Hz, 1 H) 8.16 (s, 1 H) 8.33 (s, 1 H) 9.37 (s, 1 H); HRMS (ESI+) calcd for C₂₀H₁₄ClFN₆ (MH+) 393.10252, found 393.1024.

### Example 14

### 6-(3-Amino-benzylamino)-4-(3-chloro-4-fluoro-phenylamino)-quinoline-3-carbonitrile

4-(3-chloro-4-fluoro-phenylamino)-6-(3-nitro-benzylamino)-quinoline-3-carbonitrile (200 mg, 0.45 mmol) was suspended in ethanol (10 mL) and tin chloride dihydrate (505 mg, 2.23 mmol) was added and heated at reflux for 12 hours or until complete by TLC. It was diluted with water and NaHCO₃ was added until basic then extracted with CHCl₃, washed with brine, dried over Mg₂SO₄. The residue was purified via flash column chromatography eluting with 0-7.5% MeOH in CH₂Cl₂ to obtain 6-(3-amino-benzylamino)-4-(3-chloro-4-fluoro-phenylamino)-quinoline-3-carbonitrile (136 mg) in 73% yield: 1H NMR (400 MHz, DMSO-D6) δ ppm 4.21 (d, J=5.56 Hz, 2 H) 5.04 (s, 2 H) 6.42-6.46 (m, 1 H) 6.50 (dd, J=7.71, 1.14 Hz, 1 H) 6.57 (t, J=1.64 Hz, 1 H) 6.73 (t, J=5.56 Hz, 1 H) 6.96 (t, J=7.71 Hz, 1 H) 7.12 (d, J=2.53 Hz, 1 H) 7.18-7.24 (m, 1 H) 7.35 (dd, J=9.09, 2.27 Hz, 1 H) 7.39-7.45 (m, 2 H) 7.68 (d, J=9.09 Hz, 1 H) 8.31 (s, 1 H) 9.32 (s, 1 H); HRMS (ESI+) calcd for C₂₃H₁₇ClFN₅ (MH+) 418.12293, found 418.1227.

### Example 15

### 4-(3-Chloro-4-fluoro-phenylamino)-6-[(5-methyl-3H-imidazol-4-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 4-methyl-5-imidazole carboxaldehyde (168 mg, 1.53 mmol) and NaCNBH₃ (48 mg, 0.77 mmol) in 210 mL EtOH. The crude product was purified by preparative HPLC, and lyophilized to give the product as a solid (197 mg, 76%): 1H NMR (400 MHz, DMSO-D6) δ ppm 2.16 (s, 3 H) 4.15 (d, J=4.29 Hz, 2 H) 6.39 (t, J=4.55 Hz, 1 H) 7.18 (s, 1 H) 7.21-7.27 (m, 1 H) 7.35-7.49 (m, 3 H) 7.50 (s, 1 H) 7.67 (d, J=9.35 Hz, 1 H) 8.16 (s, 1 H) 8.32 (s, 1 H) 9.35 (s, 1 H); HRMS (ESI+) calcd for C₂₁H₁₆CIFN₆ (MH+) 407.11818, found 407.118.

### Example 16

### N-(4-{[4-(3-Chloro-4-fluoro-phenylamino)-3-cyano-quinolin-6-ylamino]-methyl}-phenyl)-acetamide

Following the procedure described above in Example 4, 6-amino-4-(3-chloro-4-fluoro-phenylamino)-quinoline-3-carbonitrile (200 mg, 0.64 mmol) was reacted with 4-acetimidobenzaldehyde (208 mg, 1.28 mmol) and NaCNBH₃ (48 mg, 0.77 mmol) in 8 mL EtOH. The crude product was purified by preparative HPLC, and lyophilized to give the product as a solid (70 mg, 24%): 1H NMR (400 MHz, DMSO-D6) δ ppm 2.02 (s, 3 H) 4.30 (d, J=5.31 Hz, 2 H) 6.79 (t, J=5.68 Hz, 1 H) 7.13 (d, J=2.02 Hz, 1 H) 7.18-7.24 (m, 1 H) 7.29 (d, J=8.59 Hz, 2 H) 7.35 (dd, J=8.84, 2.27 Hz, 1 H) 7.38-7.46 (m, 2 H) 7.52 (d, J=8.59 Hz, 2 H) 7.67-7.71 (m, J=9.09 Hz, 1 H) 8.32 (s, 1 H) 9.32 (s, 1 H) 9.91 (s, 1 H); HRMS (ESI+) calcd for C₂₅H₁₉CIFN₅O (MH+) 460.13349, found 460.1337.

### Example 17

### 4-(3-Chloro-4-fluoro-phenylamino)-6-(4-nitro-benzylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 4-nitrobenzaldehyde (231 mg, 1.53 mmol) and NaCNBH₃ (48 mg, 0.77 mmol) in 8 mL EtOH. The crude product was purified by preparative HPLC, and lyophilized to give the product as a solid (161 mg, 56%): 1H NMR (400 MHz, DMSO-D6) δ ppm 4.55 (d, J=6.06 Hz, 2 H) 7.04 (d, J=2.53 Hz, 1 H) 7.07-7.17 (m, 2 H) 7.32-7.39 (m, 3 H) 7.60 (d, J=8.84 Hz, 2 H) 7.73 (d, J=9.09 Hz, 1 H) 8.16-8.23 (m, 2 H) 8.35 (s, 1 H) 9.27 (s, 1 H); HRMS (ESI+) calcd for C₂₃H₁₅CIFN₅O₂ (MH+) 448.09711, found 448.0969.

### Example 18

### N-(3-{[4-(3-Chloro-4-fluoro-phenylamino)-3-cyano-quinolin-6-ylamino]-methyl}-phenyl)-methanesulfonamide

6-(3-amino-benzylamino)-4-(3-chloro-4-fluoro-phenylamino)-quinoline-3-carbonitrile (100 mg, 0.24 mmol, prepared according to the procedure described in Example 14) was taken up in NMP (3 mL), and triethylamine (38 uL, 0.28 mmol) was added. The reaction was cooled using and ice-EtOH bath, and MeSO₂Cl (20 uL, 0.26 mmol) was added. After 12 hours, the solvent was evaporated and the solid was purified via prep-HPLC to obtain N-(3-{[4-(3-Chloro-4-fluoro-phenylamino)-3-cyano-quinolin-6-ylamino]-methyl}-phenyl)-methanesulfonamide (25 mg) in 21% yield: 1H NMR (400 MHz, DMSO-D6) δ ppm 2.92 (s, 3 H) 4.37 (d, J=5.81 Hz, 1 H) 6.88 (t, J=5.68 Hz, 1 H) 7.06-7.13 (m, 2 H) 7.15 (d, J=2.27 Hz, 1 H) 7.18-7.23 (m, 1 H) 7.25 (t, J=1.77 Hz, 1 H) 7.28 (t, J=7.83 Hz, 1 H) 7.35 (dd, J=9.09, 2.53 Hz, 1 H) 7.38-7.45 (m, 2 H) 7.70 (d, J=9.09 Hz, 1 H) 8.31 (s, 1 H) 9.30 (s, 1 H) 9.73 (s, 1 H); HRMS (ESI+) calcd for C₂₄H₁₉ClFN₅O₂S (MH+) 496.10048, found 496.1001.

### Example 19

### 4-(3-Chloro-4-fluoro-phenylamino)-6-(4-cyano-benzylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 4-cyanobenzaldehyde (72 mg, 0.64 mmol) and NaCNBH₃ (48 mg, 0.77 mmol) in 8 mL EtOH. The crude product was purified by preparative HPLC, and lyophilized to give the product as a solid (195 mg, 71%): 1H NMR (400 MHz, DMSO-D6) δ ppm 4.49 (d, J=6.06 Hz, 2 H) 7.00-7.08 (m, 2 H) 7.13-7.19 (m, 1 H) 7.33-7.41 (m, 3 H) 7.54 (d, J=8.34 Hz, 2 H) 7.72 (d, J=9.09 Hz, 1 H) 7.76-7.82 (m, 2 H) 8.34 (s, 1 H) 9.27 (s, 1 H); HRMS (ESI+) calcd for C₂₄H₁₅ClFN₅ (MH+) 428.10728, found 428.1074.

### Example 20

### 4-(3-Chloro-4-fluoro-phenylamino)-6-(3-cyano-4-dimethylamino-2-fluoro-benzylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 3-cyano-4-dimethylamino-2-fluorobenzaldehyde (1 23 mg, 0.64 mmol) and NaCNBH₃ (48 mg, 0.77 mmol) in 8 mL EtOH. The crude product was purified by preparative HPLC, and lyophilized to give the product as a solid (166 mg,53%): 1H NMR (400 MHz, DMSO-D6) δ ppm 3.02 (s, 6 H) 4.32 (d, J=5.56 Hz, 2 H) 6.75-6.79 (m, 2 H) 7.12 (d, J=2.53 Hz, 1 H) 7.18-7.24 (m, 1 H) 7.34 (dd, J=8.97, 2.40 Hz, 1 H) 7.38-7.45 (m, 2 H) 7.49 (t, J=8.97 Hz, 1 H) 7.72 (d, J=9.09 Hz, 1 H) 8.34 (s, 1 H) 9.32 (s, 1 H).

### Example 21

### 4-(3-Chloro-4-fluoro-phenylamino)-6-(2-cyano-benzylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 2-cyanobenzaldehyde (84 mg, 0.64 mmol) and NaCNBH₃ (48 mg, 0.77 mmol) in 8 mL EtOH. The crude product was purified by preparative HPLC, and lyophilized to give the product as a solid (75 mg, 27%): 1H NMR (400 MHz, DMSO-D6) δ ppm 4.56 (d, J=5.56 Hz, 2 H) 6.95 (t, J=5.81 Hz, 1 H) 7.12 (d, J=2.27 Hz, 1 H) 7.15-7.21 (m, 1 H) 7.34-7.44 (m, 3 H) 7.46-7.51 (m, 1 H) 7.56 (d, J=7.07 Hz, 1 H) 7.64-7.70 (m, 1 H) 7.74 (d, J=8.84 Hz, 1 H) 7.86 (dd, J=7.45, 1.14 Hz, 1 H) 8.36 (s, 1 H) 9.33 (s, 1 H).

### Example 22

### 4-(3-Chloro-4-fluoro-phenylamino)-6-{[1-(2-morpholin-4-yl-ethyl)-1H-imidazol-2-ylmethyl]-amino}-quinoline-3-carbonitrile

Step 1: 2-lmidazolecarboxaldehyde (750 mg, 7.81 mmol), sodium carbonate (827 mg, 7.81 mmol), N-(2-chloroethyl)morpholine hydrochloride (726 mg, 3.9 mmol), and sodium iodide (585 mg, 3.9 mmol) were taken up in DMF in a sealed tube and heated at 100° C. for 18 hours. The reaction was filtered and diluted with ethyl acetate, washed with brine, dried over Mg₂SO₄ and stripped. 400 mg of 3:1 (by LC/MS) mixture of 1-(2-Morpholin-4-yl-ethyl)-1H-imidazole-2-carbaldehyde and 2-Imidazolecarboxaldehyde was obtained and carried on crude to the reductive amination.

Step 2: Following the procedure described above in Example 4, 6-amino-4-(3-chloro-4-fluoro-phenylamino)-quinoline-3-carbonitrile (300 mg, 0.96 mmol) was reacted with 1-(2-morpholin-4-yl-ethyl)-1 H-imidazole-2-carbaldehyde (crude mixture) (187 mg, 0.96 mmol) and NaCNBH₃ (73 mg, 1.15 mmol) in 8 mL EtOH. The crude product was purified by preparative HPLC, and lyophilized to give the product as a solid (285 mg, 59%): 1H NMR (400 MHz, H2O+D2O) δ ppm 3.12 (s, 2 H) 3.43 (t, J=10.36 Hz, 4 H) 3.72-3.80 (m, 4 H) 4.48-4.55 (m, 2 H) 4.80 (s, 2 H) 7.20 (d, J=2.27 Hz, 1 H) 7.22-7.29 (m, 3 H) 7.41-7.50 (m, 3 H) 7.71 (d, J=9.09 Hz, 1 H) 8.47 (s, 1 H).

### Example 23

### 4-(3-Chloro-4-fluoro-phenylamino)-6-[(2-ethyl-5-methyl-3H-imidazol-4-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 2-ethyl-4-methyl-1H-imidazole-5-carboxaldehyde (88 mg, 0.64 mmol) and NaCNBH₃ (50 mg, 0.77 mmol) in 8 mL EtOH. The crude product was purified by preparative HPLC, and lyophilized to give the product as a solid (189 mg, 68%): 1H NMR (400 MHz, DMSO-D6) δ ppm 1.17 (t, J=7.58 Hz, 3 H) 2.11 (s, 3 H) 2.55 (q, J=7.58 Hz, 2 H) 4.09 (d, J=4.29 Hz, 2 H) 6.36 (t, J=4.80 Hz, 1 H) 7.16 (d, J=2.27 Hz, 1 H) 7.21-7.26 (m, 1 H) 7.36-7.48 (m, 3 H) 7.67 (d, J=9.09 Hz, 1 H) 8.17 (s, 1 H) 8.32 (s, 1 H) 9.34 (s, 1 H); HRMS (ESI+) calcd for C₂₃H₂₀ClFN₆ (MH+) 435.14947, found 435.1504.

### Example 24

### 6-[3-Bromo-4-(2-methoxy-ethoxy)-benzylamino]-4-(3-chloro-4-fluoro-phenylamino)-quinoline-3-carbonitrile

Step 1: 3-bromo-4-hydroxybenzaldehyde (1 g, 4.97 mmol) was taken up in DMF (20 mL), then sodium hydride 60% (200 mg, 4.97 mmol) was added followed by 2-bromoethylmethylether (514 uL, 5.47 mmol) and heated at 50° C. for 24 hours. Then the mixture was diluted with ethyl acetate, washed with brine, dried over Mg₂SO₄. The residue was purified via flash column chromatography to obtain 3-bromo-4-(2-methoxy-ethoxy)-benzaldehyde (900 mg) in a 70% yield: 1H NMR (400 MHz, DMSO-D6) δ ppm 3.35 (s, 3 H) 3.71-3.75 (m, 2 H) 4.30-4.34 (m, 2 H) 7.33 (d, J=8.59 Hz, 1 H) 7.92 (dd, J=8.34, 2.02 Hz, 1 H) 8.11 (d, J=2.02 Hz, 1 H) 9.86 (s, 1 H).

Step 2: Following the procedure described above in Example 4, 6-amino-4-(3-chloro-4-fluoro-phenylamino)-quinoline-3-carbonitrile (200 mg, 0.64 mmol) was reacted with 3-bromo-4-(2-methoxy-ethoxy)-benzaldehyde (166 mg, 0.64 mmol) and NaCNBH₃ (50 mg, 0.77 mmol) in 8 mL EtOH. The crude product was purified by preparative HPLC, and lyophilized to give the product as a solid (330 mg, 93%): 1H NMR (400 MHz, DMSO-D6) δ ppm 3.32 (s, 3 H) 3.65-3.69 (m, 2 H) 4.11-4.16 (m, 2 H) 4.31 (d, J=5.81 Hz, 2 H) 6.86 (t, J=6.19 Hz, 1 H) 7.07 (d, J=8.34 Hz, 1 H) 7.12 (d, J=2.02 Hz, 1 H) 7.18-7.23 (m, 1 H) 7.30-7.36 (m, 2 H) 7.38-7.45 (m, 2 H) 7.60 (d, J=2.02 Hz, 1 H) 7.70 (d, J=9.09 Hz, 1 H) 8.32 (s, 1 H) 9.30 (s, 1 H); HRMS (ESI+) calcd for C₂₆H₂₁BrClFN₄O₂ (MH+) 555.05932, found 555.0606.

### Example 25

### 4-(3-Chloro-4-fluoro-phenylamino)-6-[3-cyano-4-(2-methoxy-ethoxy)-benzylamino]-quinoline-3-carbonitrile

6-[3-bromo-4-(2-methoxy-ethoxy)-benzylamino]-4-(3-chloro-4-fluoro-phenylamino)-quinoline-3-carbonitrile (150 mg, 0.27 mmol, prepared according to the procedures described in Example 24 above), zinc(ll) cyanide (127 mg, 1.08 mmol), palladium tetrakis (93 mg, 0.08 mmol) were taken up in DMF (2 mL) and heated 150° C. in the microwave for 60 minutes. The mixture was then diluted with ethyl acetate, washed with brine, dried over Mg₂SO₄ and purified via flash column chromatography to obtain 4-(3-chloro-4-fluoro-phenylamino)-6-[3-cyano-4-(2-methoxy-ethoxy)-benzylamino]-quinoline-3-carbonitrile (108 mg) in 80 % yield: 1H NMR (400 MHz, DMSO-D6) δ ppm 3.32 (s, 3 H) 3.67-3.70 (m, 2 H) 4.22-4.26 (m, 2 H) 4.34 (d, J=5.56 Hz, 2 H) 6.88 (t, J=6.19 Hz, 1 H) 7.11 (d, J=2.53 Hz, 1 H) 7.18-7.24 (m, 2 H) 7.34 (dd, J=9.09, 2.53 Hz, 1 H) 7.38-7.44 (m, 2 H) 7.63 (dd, J=8.72, 2.15 Hz, 1 H) 7.69-7.73 (m, 2 H) 8.33 (s, 1 H) 9.31 (s, 1 H); HRMS (ESI+) calcd for C₂₇H₂₁ClFN₅O₂ (MH+) 502.14406, found 502.145.

### Example 26

### (2-Bromo-4-{[4-(3-chloro-4-fluoro-phenylamino)-3-cyano-quinolin-6-ylamino]-methyl}-phenoxy)-acetic acid tert-butyl ester

Step 1: 3-bromo-4-hydroxybenzaldehyde (1 g, 4.97 mmol) was reacted with tert-butylbromoacetate (734 uL, 4.97 mmol) according to the procedure described above in Example 25, step 1, to obtain (2-bromo-4-formyl-phenoxy)-acetic acid tert-butyl ester (1.16 g) in 74% yield: 1H NMR (400 MHz, DMSO-D6) δ ppm 1.43 (s, 9 H) 4.95 (s, 2 H) 7.20 (d, J=8.59 Hz, 1 H) 7.89 (dd, J=8.34, 2.02 Hz, 1 H) 8.12 (d, J=1.77 Hz, 1 H) 9.86 (s,1 H).

Step 2: Following the procedure described above in Example 4, 6-amino-4-(3-chloro-4-fluoro-phenylamino)-quinoline-3-carbonitrile (250 mg, 0.80 mmol) was reacted with (2-bromo-4-formyl-phenoxy)-acetic acid tert-butyl ester (251 mg, 0.80 mmol) and NaCNBH₃ (60 mg, 0.96 mmol) in 5 mL EtOH. The crude product was purified by preparative HPLC, and lyophilized to give the product as a solid (279 mg, 57%): 1H NMR (400 MHz, DMSO-D6) δ ppm 1.39 (s, 9 H) 4.32 (d, J=5.81 Hz, 2 H) 4.74 (s, 2 H) 6.86 (t, J=5.94 Hz, 1 H) 6.92 (d, J=8.59 Hz, 1 H) 7.13 (d, J=2.02 Hz, 1 H) 7.18-7.24 (m, 1 H) 7.28-7.36 (m, 2 H) 7.38-7.46 (m, 2 H) 7.61 (d, J=1.77 Hz, 1 H) 7.70 (d, J=9.09 Hz, 1 H) 8.32 (s, 1 H) 9.31 (s, 1 H).

### Example 27

### 4-(3-Chloro-4-fluoro-phenylamino)-6-[(1H-pyrazol-3-ylmethyl)-amino]-quinoline-3-carbonitrile

Following the procedure described above in Example 4, 6-amino-4-(3-chloro-4-fluoro-phenylamino)-quinoline-3-carbonitrile (179 mg, 0.57 mmol) was reacted with 1H-pyrazole-3-carbaldehyde (CL-201667) (55 mg , 0.57 mmol) and NaCNBH₃ (43 mg, 0.69 mmol) in 8 mL EtOH. The crude product was purified by preparative HPLC, and lyophilized to give the product as a solid (105 mg, 47%): 1H NMR (400 MHz, DMSO-D6) δ ppm 4.35 (d, J=5.05 Hz, 2 H) 6.25 (d, J=1.77 Hz, 1 H) 6.62 (t, J=5.18 Hz, 1 H) 7.20-7.28 (m, 2 H) 7.35-7.50 (m, 3 H) 7.62 (s, 1 H) 7.69 (d, J=9.09 Hz, 1 H) 8.32 (s, 1 H) 9.36 (s, 1 H) 12.64 (s, 1 H).

### Example 28

### 4-(3-Chloro-4-fluoro-phenylamino)-8-methoxy-6-[(pyridin-3-ylmethyl)-amino]-quinoline-3-carbonitrile

Step 1: 2-methoxy-4-nitroaniline (25 g, 149 mmol) and ethyl(ethoxymethylene) cyanoacetate (26.4 g, 156 mmol) was dissolved in DMF (125 mL), then cesium carbonate (97 g, 297 mmol) was added, the reaction turned red and was left to stir at RT for 18 hours or until complete by LC/MS. The reaction was poured into 20× volume of water and a yellow solid precipitated out. The solid was collected by suction filtration, rinsed with water and hexanes then triturated for 18 hours in tertbutylmethylether (500 mL), the filtered to obtain 2-cyano-3-(2-methoxy-4-nitro-phenylamino)-acrylic acid ethyl ester (31.2 g) in 72% yield: 1H NMR (400 MHz, DMSO-D6) δ ppm 1.28 (t, J=7.20 Hz, 3 H) 4.05 (s, 3 H) 4.27 (q, J=7.16 Hz, 2 H) 7.87-7.97 (m, 3 H) 8.80 (d, J=13.39 Hz, 1 H) 11.12 (d, J=13.39 Hz, 1 H).

Step 2: 2-Cyano-3-(2-methoxy-4-nitro-phenylamino)-acrylic acid ethyl ester (6.25 g, 21 mmol) was suspended in Dowtherm A (25 mL) and heated at 260° C. for 18 hours. The reaction is cooled to RT, the poured into 1.5 L of hexanes and stirred for 1 hour. The dark brown solid is collected via suction filtration to obtain 8-methoxy-6-nitro-4-oxo-1,4-dihydro-quinoline-3-carbonitrile (4.62 g, crude 66% desired product by LC/MS) in 88% yield: 1H NMR (400 MHz, DMSO-D6) δ ppm 4.13 (s, 3 H) 8.01 (d, J=2.53 Hz, 1 H) 8.42 (d, J=2.27 Hz, 1 H) 8.64 (s, 1 H).

Step 3: 8-methoxy-6-nitro-4-oxo-1,4-dihydro-quinoline-3-carbonitrile (2 g, 8.2 mmol) was suspended in POCl₃ (15 mL) and the reaction was carried out according to Example 4, step 2. The residue was purified via flash column chromatography to obtain 4-Chloro-8-methoxy-6-nitro-quinoline-3-carbonitrile (400 mg) in 19% yield: 1H NMR (400 MHz, DMSO-D6) δ ppm 4.16 (s, 3 H) 8.06 (d, J=2.27 Hz, 1 H) 8.59 (d, J=2.27 Hz, 1 H) 9.34 (s, 1 H).

Step 4: 4-chloro-8-methoxy-6-nitro-quinoline-3-carbonitrile (250 mg, 1.02 mmol) and 3-chloro-4-fluoroaniline (17 g, 1.2 mmol) were suspended in ethanol (10 mL) and the reaction carried out according to Example 4, step 3. The residue was purified via flash column chromatography to obtain 4-(3-chloro-4-fluoro-phenylamino)-8-methoxy-6-nitro-quinoline-3-carbonitrile (200 mg) in 53% yield: 1 H NMR (400 MHz, DMSO-D6) δ ppm 4.09 (s, 3 H) 7.40 (s, 1 H) 7.50 (s, 1 H) 7.66 (s, 1 H) 7.95 (d, J=2.27 Hz, 1 H) 8.70 (s, 1 H) 9.10 (s, 1 H) 10.46 (s,1 H).

Step 5: 4-(3-Chloro-4-fluoro-phenylamino)-8-methoxy-6-nitro-quinoline-3-carbonitrile (390 mg, 1.05 mmol) was suspended in ethanol (4 mL), then tin chloride dihydrate (948 mg, 4.19 mmol, 4 eq) was added and the reaction heated in the microwave at 110° C. for 5 minutes. The reaction was diluted with water, then NaHCO₃ is added until the pH was basic. The solution was extracted with chloroform, washed with brine, treated with activated carbon, dried over Mg2SO4, and stripped to obtain 6-amino-4-(3-chloro-4-fluoro-phenylamino)-8-methoxy-quinoline-3-carbonitrile (332 mg) in 93% yield: 1H NMR (400 MHz, DMSO-D6) δ ppm 3.88 (s, 3 H) 5.76 (s, 2 H) 6.72 (d, J=20.46 Hz, 2 H) 7.06-7.15 (m, 1 H) 7.31 (d, J=4.55 Hz, 1 H) 7.36 (t, J=9.09 Hz, 1 H) 8.27 (s, 1 H) 9.22 (s, 1 H).

Step 6: Following the procedure described above in Example 4, 6-amino-4-(3-chloro-4-fluoro-phenylamino)-8-methoxy-quinoline-3-carbonitrile (90 mg, 0.26 mmol) was reacted with pyridine-3-carbaldehyde (25 uL, 0.26 mmol) and NaCNBH₃ (20 mg, 0.32 mmol) in 3 mL EtOH. The crude product was purified by preparative HPLC, and lyophilized to give the product as a solid (24 mg, 21 %): 1H NMR (400 MHz, DMSO-D6) δ ppm 3.87 (s, 3 H) 4.41 (d, J=5.56 Hz, 2 H) 6.73 (d, J=2.02 Hz, 1 H) 6.80-6.85 (m, 2 H) 7.14-7.20 (m, 1 H) 7.33-7.42 (m, 3 H) 7.74-7.79 (m, 1 H) 8.26 (s,1 H) 8.46 (dd, J=4.80,1.52 Hz, 1 H) 8.60 (d, J=2.02 Hz, 1 H) 9.18 (s, 1 H).

### Example 29

### 4-(3-Chloro-4-fluoro-phenylamino)-8-methoxy-6-(2-morpholin-4-yl-ethylamino)-quinoline-3-carbonitrile

Following the procedure described above in Example 4, 6-amino-4-(3-chloro-4-fluoro-phenylamino)-8-methoxy-quinoline-3-carbonitrile (200 mg, 0.58 mmol), was reacted with NaCNBH₃ (44 mg, 0.69 mmol) and morpholin-4-yl-acetaldehyde (prepared by heating the corresponding dimethyl acetal (256 mg, 1.45 mmol) in 2.OmL concentrated HCI for 5 minutes in a microwave reactor at 110° C., then neutralizing the mixture with solid K₂CO₃ until pH=6). The crude product was purified by preparative HPLC, and lyophilized to give the product as a solid (35 mg, 13%): 1H NMR (400 MHz, DMSO-D6) δ ppm 2.40-2.45 (m, 4 H) 2.54 (t, J=6.69 Hz, 2 H) 3.20-3.26 (m, 2 H) 3.57-3.61 (m, 4 H) 3.87 (s, 3 H) 6.11 (t, J=5.56 Hz, 1 H) 6.61 (d, J=2.02 Hz, 1 H) 6.83 (d, J=2.02 Hz, 1 H) 7.16-7.22 (m, 1 H) 7.37-7.44 (m, 2 H) 8.25 (s, 1 H) 9.19 (s, 1 H).

### Example 30

### 4-(3-Chloro-4-fluoro-phenylamino)-6-[(1-oxy-pyridin-3-ylmethyl)-amino]-quinoline-3-carbonitrile

Step 1: 3-Pyridylcarbinol-N-oxide (500 mg, 4 mmol) and manganese(IV) oxide (2.1 g, 24 mmol) was taken up in CHCl₃ (15 mL) and stirred at RT for 120 hours, then filtered and stripped to obtain 1-oxy-pyridine-3-carbaldehyde (80 mg) in 16% yield: 1H NMR (400 MHz, DMSO-D6) δ ppm 7.59-7.63 (m, 1 H) 7.76 (dt, J=7.83, 1.14 Hz, 1 H) 8.45-8.49 (m, 1 H) 8.66-8.68 (m, 1 H) 9.97 (s, 1 H).

Step 2: Following the procedure described above in Example 4, 6-amino-4-(3-chloro-4-fluoro-phenylamino)-quinoline-3-carbonitrile (177 mg, 0.57 mmol) was reacted with 1-oxy-pyridine-3-carbaldehyde (80 mg , 0.65 mmol) and NaCNBH₃ (49 mg, 0.78 mmol) in 5 mL EtOH. The crude product was purified by preparative HPLC, and lyophilized to give the product in quantitative yield: 1H NMR (400 MHz, DMSO-D6) δ ppm 4.40 (d, J=6.57 Hz, 2 H) 6.95 (t, J=6.19 Hz, 1 H) 7.13 (d, J=Hz, 1 H) 7.20-7.25 (m, 1 H) 7.29-7.48 (m, 5 H) 7.73 (d, J=9.09 Hz, 1 H) 8.08-8.12 (m, 1 H) 8.21 (s, 1 H) 8.33 (s, 1 H) 9.30 (s, 1 H).

### Example 31

### 4-(3-Chloro-4-fluoro-phenylamino)-6-[(5-methyl-1 H-pyrazol-3-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 5-methyl-1H-pyrazole-3-carbaldehyde (CL-83045) (53 mg , 0.48 mmol) and NaCNBH₃ (36 mg, 0.58 mmol) in 5 mL EtOH. The crude product was purified by preparative HPLC, and lyophilized to give the product as a solid (58 mg, 30%): 1H NMR (400 MHz, DMSO-D6) δ ppm 2.18 (s, 3 H) 4.26 (d, J=5.31 Hz, 2 H) 5.98 (s,1 H) 6.57 (t, J=5.18 Hz, 1 H) 7.20 (d, J=2.27 Hz, 1 H) 7.22-7.27 (m, 1 H) 7.37 (dd, J=9.09, 2.27 Hz, 1 H) 7.40-7.48 (m, 2 H) 7.68 (d, J=9.09 Hz, 1 H) 8.32 (s, 1 H) 9.35 (s, 1 H).

### Example 32

### 4-(3-Hydroxy-4-methyl-phenylamino)-8-methoxy-6-[(pyridin-3-ylmethyl)-amino]-quinoline-3-carbonitrile

Following the procedure described above in Example 4, 6-amino-4-(3-hydroxy-4-methyl-phenylamino)-8-methoxy-quinoline-3-carbonitrile (67 mg, 0.21 mmol) was reacted with pyridine-3-carbaldehyde (20 uL, 0.21 mmol) and NaCNBH₃ (16 mg, 0.25 mmol) in 5 mL EtOH. The crude product was purified by preparative HPLC, and lyophilized to give the product as a solid (44 mg, 51 %): 1 H NMR (400 MHz, DMSO-D6) δ ppm 2.11 (s, 3 H) 3.85 (s, 3 H) 4.40 (d, J=6.32 Hz, 2 H) 6.52 (s, 1 H) 6.58 (s, 1 H) 6.71 (s, 1 H) 6.81 (d, J=15.16 Hz, 2 H) 7.01 (d, J=8.84 Hz, 1 H) 7.36 (dd, J=7.71, 4.67 Hz, 1 H) 7.77 (d, J=9.09 Hz, 1 H) 8.16 (s, 1 H) 8.46 (d, J=5.05 Hz, 1 H) 8.61 (s, 1 H) 8.98 (s,1 H).

### Example 33

### 4-(3-Chloro-4-fluoro-phenylamino)-6-[(2,5-dimethyl-2H-pyrazol-3-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with 1,3-dimethyl-1 H-pyrazole-5-carbaldehyde (60 mg , 0.48 mmol) and NaCNBH₃ (36 mg, 0.58 mmol) in 15 mL EtOH. The crude product was purified by preparative HPLC, and lyophilized to give the product as a solid (1 28 mg, 63%): 1H NMR (400 MHz, DMSO-D6) δ ppm 2.07 (s, 3 H) 3.72 (s, 3 H) 4.33 (d, J=5.31 Hz, 2 H) 6.01 (s, 1 H) 6.72 (t, J=5.43 Hz, 1 H) 7.18-7.26 (m, 2 H) 7.35 (dd, J=9.09, 2.27 Hz, 1 H) 7.39-7.48 (m, 2 H) 7.71 (d, J=9.09 Hz, 1 H) 8.34 (s, 1 H) 9.34 (s, 1 H).

### Example 34

### (4-{[4-(3-Chloro-4-fluoro-phenylamino)-3-cyano-quinolin-6-ylamino]-methyl}-2-cyano-phenoxy)-acetic acid

Step 1: (2-bromo-4-{[4-(3-chloro-4-fluoro-phenylamino)-3-cyano-quinolin-6-ylamino]-methyl}-phenoxy)-acetic acid tert-butyl ester (260 mg, 0.44 mmol, prepared according to the procedure described in Example 26) was converted to (4-{[4-(3-Chloro-4-fluoro-phenylamino)-3-cyano-quinolin-6-ylamino]-methyl}-2-cyano-phenoxy)-acetic acid tert-butyl ester according to the procedure described in Example 26 to obtain desired product in 90% yield.

Step 2: (4-{[4-(3-Chloro-4-fluoro-phenylamino)-3-cyano-quinolin-6-ylamino]-methyl}-2-cyano-phenoxy)-acetic acid tert-butyl ester (100 mg, 0.18 mmol), cerium chloride heptahydrate (134 mg, 0.36 mmol) and potassium iodide (40 mg, 0.23 mmol) were taken up in acetonitrile (10 mL) and heated in the microwave at 150° C. for 30 min, then filtered and purified via prep-HPLC to obtain (4-{[4-(3-Chloro-4-fluoro-phenylamino)-3-cyano-quinolin-6-ylamino]-methyl}-2-cyano-phenoxy)-acetic acid (30 mg) in 33% yield: 1H NMR (400 MHz, DMSO-D6) δ ppm 4.33 (d, J=5.56 Hz, 2 H) 4.83 (s, 2 H) 6.84 (t, J=6.19 Hz, 1 H) 7.08-7.15 (m, 2 H) 7.19-7.24 (m, 1 H) 7.33 (dd, J=8.97, 2.15 Hz, 1 H) 7.37-7.46 (m, 2 H) 7.61 (dd, J=8.59, 2.27 Hz, 1 H) 7.68-7.74 (m, 2 H) 8.32 (s, 1 H) 9.31 (s, 1 H) 13.20 (s, 1 H).

### Example 35

### 4-(3-Chloro-4-fluoro-phenylamino)-6-[(3-thiophen-2-yl-1H-pyrazol-4-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with thiophen-2-yl-1H-pyrazole-4-carbaldehyde (114 mg, 0.64 mmol) and NaCNBH₃ (48 mg, 0.78 mmol) in 15 mL EtOH. The crude product was purified by preparative HPLC, and lyophilized to give the product as a solid (108 mg, 36%): 1H NMR (400 MHz, DMSO-D6) δ ppm 4.27 (d, J=5.05 Hz, 2 H) 6.53 (t, J=5.05 Hz, 1 H) 7.07 (s, 1 H) 7.19 (d, J=2.53 Hz, 1 H) 7.22-7.28 (m, 1 H) 7.35-7.49 (m, 3 H) 7.65-7.73 (m, 2 H) 8.13 (s, 1 H) 8.31 (s, 1 H) 9.35 (s, 1 H) 12.51 (s, 1 H).

### Example 36

### 6-Benzylamino-4-(3-chloro-4-fluoro-phenylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with benzaldehyde (65 uL, 0.64 mmol) and NaCNBH₃ (48 mg, 0.78 mmol) in 10 mL EtOH. The crude product was purified by preparative HPLC, and lyophilized to give the product as a solid (202 mg, 76%): 1H NMR (400 MHz, DMSO-D6) δ ppm 4.46 (s, 2 H) 7.21 (s, 1 H) 7.23-7.29 (m, 1 H) 7.34 (t, J=7.33 Hz, 2 H) 7.38-7.54 (m, 6 H) 7.68 (dd, J=6.57, 2.53 Hz, 1 H) 7.80 (d, J=9.09 Hz, 1 H) 8.66 (s, 1 H) 10.52 (s, 1 H).

### Example 37

### 4-(3-Chloro-4-fluoro-phenylamino)-6-[(pyridin-3-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with pyridine-3-carbaldehyde (60 uL, 0.64 mmol) and NaCNBH₃ (48 mg, 0.78 mmol) in lOmL EtOH. The crude product was purified by preparative HPLC, and lyophilized to give the product as a solid (93 mg, 36%): 1H NMR (400 MHz, DMSO-D6) δ ppm 4.42 (d, J=5.56 Hz, 2 H) 6.90 (t, J=5.81 Hz, 1 H) 7.17 (d, J=2.27 Hz, 1 H) 7.18-7.24 (m, 1 H) 7.33-7.46 (m, 4 H) 7.71 (d, J=9.09 Hz, 1 H) 7.75-7.80 (m, 1 H) 8.33 (s, 1 H) 8.47 (dd, J=4.80,1.52 Hz, 1 H) 8.61 (d, J=2.02 Hz, 1 H) 9.31 (s, 1 H).

### Example 38

### 4-(3-Chloro-4-fluoro-phenylamino)-6-[(1,3-dimethyl-5-morpholin-4-yl-1H-pyrazol-4-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 1,3-dimethyl-5-morpholin-4-yl-1H-pyrazole-4-carbaldehyde (134 mg, 0.64 mmol) and NaCNBH₃ (48 mg, 0.78 mmol) in 15 mL EtOH. The crude product was purified by preparative HPLC, and lyophilized to give the product as a solid (96mg, 30%): 1H NMR (400 MHz, DMSO-D6) 6 ppm 2.07 (s, 3 H) 3.00-3.05 (m, 4 H) 3.60 (s, 3 H) 3.63-3.69 (m, 4 H) 4.05 (d, J=4.04 Hz, 2 H) 6.30 (t, J=4.17 Hz, 1 H) 7.15 (d, J=2.02 Hz, 1 H) 7.23-7.29 (m, 1 H) 7.34 (dd, J=9.22, 1.89 Hz, 1 H) 7.43 (t, J=8.97 Hz, 1 H) 7.48 (dd, J=6.06, 2.27 Hz, 1 H) 7.67 (d, J=9.09 Hz, 1 H) 8.32 (s,1 H) 9.35 (s, 1 H).

### Example 39

### 4-(3-Chloro-4-fluoro-phenylamino)-6-[(1-oxy-pyridin-2-ylmethyl)-amino]-quinoline-3-carbonitrile

Step 1: 2-pyridylcarbinol-N-oxide was converted to 1-Oxy-pyridine-2-carbaldehyde according to the procedure described in Example 30, and the crude product was used directly in the next step.

Step 2: Following the procedure described above in Example 4, 6-amino-4-(3-chloro-4-fluoro-phenylamino)-quinoline-3-carbonitrile (200 mg, 0.64 mmol) was reacted with 1-oxy-pyridine-2-carbaldehyde (80 mg (crude), 0.64 mmol) and NaCNBH₃ (48 mg, 0.78 mmol) in 15 mL EtOH. The crude product was purified by preparative HPLC, and lyophilized to give the product as a solid (198 mg, 74%): 1H NMR (400 MHz, DMSO-D6) δ ppm 4.58 (s, 2 H) 7.03 (s, 1 H) 7.16 (d, J=2.27 Hz, 1 H) 7.21-7.27 (m, 1 H) 7.28-7.38 (m, 3 H) 7.37-7.51 (m, 3 H) 7.76 (d, J=9.09 Hz, 1 H) 8.32 (d, J=6.06 Hz, 1 H) 8.39 (s, 1 H) 9.58 (s, 1 H).

### Example 40

### 2-{[4-(3-Chloro-4-fluoro-phenylamino)-3-cyano-quinolin-6-ylamino]-methyl}-L-pyrrolidine-1-carboxylic acid tert-butyl ester

Following the procedure described above in Example 4, 6-amino-4-(3-chloro-4-fluoro-phenylamino)-quinoline-3-carbonitrile (300 mg, 0.96 mmol) was reacted with N-(tert-butoxycarbonyl)-L-prolinal (180 uL, 0.96 mmol) and NaCNBH₃ (73 mg, 1.15 mmol) in 10 mL EtOH. The crude product was purified by preparative HPLC, and lyophilized to give the product as a solid (165 mg, 35%): 1H NMR (400 MHz, DMSO-D6) δ ppm 1.35 (s, 9 H) 1.76-1.94 (m, 4 H) 3.04 (s, 1 H) 3.20-3.36 (m, 2 H) 3.92-4.06 (m, 1 H) 6.53 (s, 1 H) 7.04-7.46 (m, 5 H) 7.50 (d, J=6.06 Hz, 1 H) 7.68 (d, J=9.35 Hz, 1 H) 8.30 (s, 1 H) 9.04 (s, 1 H).

### Example 41

### 4-(3-Chloro-4-fluoro-phenylamino)-6-[(tetrahydro-furan-2-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with tetrahydrofuran-3-carboxaldehyde (50% wt in water) (130 uL, 0.64 mmol) and NaCNBH₃ (50 mg, 0.76 mmol) in 10 mL EtOH. The crude product was purified by preparative HPLC, and lyophilized to give the product as a solid (156 mg, 61 %): 1H NMR (400 MHz, DMSO-D6) δ ppm 1.56-1.66 (m, 1 H) 1.98-2.08 (m, 1 H) 2.52-2.57 (m, 1 H) 3.06-3.12 (m, 2 H) 3.47 (dd, J=8.59, 5.56 Hz, 1 H) 3.61-3.68 (m, 1 H) 3.74-3.81 (m, 2 H) 6.45 (t, J=5.81 Hz, 1 H) 7.02 (d, J=2.27 Hz, 1 H) 7.22-7.27 (m, 1 H) 7.29 (dd, J=9.09, 2.53 Hz, 1 H) 7.39-7.48 (m, 2 H) 7.68 (d, J=9.09 Hz, 1 H) 8.30 (s, 1 H) 9.31 (s, 1 H).

### Example 42

### (4-Bromo-2-{([4-(3-chloro-4-fluoro-phenylamino)-3-cyano-quinolin-6-ylamino]-methyl}-phenoxy)-acetic acid tert-butyl ester

Step 1: 5-bromosalicylaldehyde (1 g, 5 mmol) was converted to (4-bromo-2-formyl-phenoxy)-acetic acid tert-butyl ester according to the procedure described above in Example 26 to obtain the desired product (1.2 g) in 78% yield.

Step 2: Following the procedure described above in Example 4, 6-amino-4-(3-chloro-4-fluoro-phenylamino)-quinoline-3-carbonitrile (200 mg, 0.64 mmol) was reacted with (4-bromo-2-formyl-phenoxy)-acetic acid tert-butyl ester (201 mg, 0.64 mmol) and NaCNBH₃ (50 mg, 0.76 mmol) in 10 mL EtOH. The crude product was purified by preparative HPLC, and lyophilized to give the product as a solid (150 mg, 38%): 1H NMR (400 MHz, DMSO-D6) δ ppm 1.41 (s, 9 H) 4.40 (d, J=5.56 Hz, 2 H) 4.74 (s, 2 H) 6.71 (t, J=6.06 Hz, 1 H) 6.89 (d, J=8.84 Hz, 1 H) 7.11-7.14 (m, J=1.77 Hz, 1 H) 7.16-7.21 (m, 1 H) 7.34-7.44 (m, 5 H) 7.72 (d, J=8.84 Hz, 1 H) 8.33 (s, 1 H) 9.33 (s, 1 H).

### Example 43

### 4-(3-Chloro-4-fluoro-phenylamino)-6-[(L-pyrrolidin-2-ylmethyl)-amino]-quinoline-3-carbonitrile

2-{[4-(3-chloro-4-fluoro-phenylamino)-3-cyano-quinolin-6-ylamino]-methyl}-L-pyrrolidine-1-carboxylic acid tert-butyl ester (80 mg, 0.161 mmol, prepared according to the procedure described in Example 40) was dissolved in dichloroethane (3 mL) and trifluoroacetic acid (500 uL) was added. The reaction mixture was stirred 18 hours at RT and stripped to obtain 4-(3-Chloro-4-fluoro-phenylamino)-6-[(L-pyrrolidin-2-ylmethyl)-amino]-quinoline-3-carbonitrile (98 mg) in quantitative yield: 1H NMR (400 MHz, DMSO-D6) δ ppm 1.62-1.74 (m, 1 H) 1.85-2.02 (m, 3 H) 2.10-2.21 (m, 1 H) 3.15-3.28 (m, 2 H) 3.42-3.49 (m, 2 H) 3.74-3.83 (m, 1 H) 6.71-6.79 (m, 1 H) 7.29 (d, J=1.26 Hz, 1 H) 7.36-7.44 (m, 2 H) 7.52 (t, J=8.97 Hz, 1 H) 7.65 (d, J=5.81 Hz, 1 H) 7.79 (d, J=9.09 Hz, 1 H) 8.57 (dd, J=7.71, 3.66 Hz, 1 H) 8.62 (s, 1 H) 9.00-9.10 (m, 1 H).

### Example 44

### (2-{[4-(3-Chloro-4-fluoro-phenylamino)-3-cyano-quinolin-6-ylamino]-methyl}-4-cyano-phenoxy)-acetic acid

(4-bromo-2-{[4-(3-chloro-4-fluoro-phenylamino)-3-cyano-quinolin-6-ylamino]-methyl}-phenoxy)-acetic acid tert-butyl ester (250 mg, 0.41 mmol, prepared according to Example 42) was converted to (2-{[4-(3-Chloro-4-fluoro-phenylamino)-3-cyano-quinolin-6-ylamino]-methyl}-4-cyano-phenoxy)-acetic acid according to the procedure described above in Example 26 to obtain the desired product (15 mg) in 7% yield: 1H NMR (400 MHz, DMSO-D6) δ ppm 4.46 (s, 2 H) 4.78 (s, 2 H) 6.88 (s, 1 H) 7.14 (d, J=8.59 Hz, 1 H) 7.21-7.28 (m, 2 H) 7.30-7.39 (m, 2 H) 7.43-7.54 (m, 2 H) 7.62-7.73 (m, 3 H) 7.78-7.84 (m, 1 H) 8.31 (s, 1 H).

### Example 45

### (2-{[4-(3-Chloro-4-fluoro-phenylamino)-3-cyano-quinolin-6-ylamino]-methyl)-4-cyano-phenoxy)-acetic acid tert-butyl ester

(4-bromo-2-{[4-(3-chloro-4-fluoro-phenylamino)-3-cyano-quinolin-6-ylamino]-methyl}-phenoxy)-acetic acid tert-butyl ester (250 mg, 0.41 mmol, prepared according to Example 42) was converted (2-{[4-(3-Chloro-4-fluoro-phenylamino)-3-cyano-quinolin-6-ylamino]-methyl}-4-cyano-phenoxy)-acetic acid tert-butyl ester according to the procedure described above in Example 26 to obtain desired product (15 mg) in 7% yield: 1H NMR (400 MHz, DMSO-D6) δ ppm 1.41 (s, 9 H) 4.41 (d, J=5.31 Hz, 2 H) 4.88 (s, 2 H) 6.72 (t, J=5.68 Hz, 1 H) 7.08-7.12 (m, 2 H) 7.15-7.20 (m, 1 H) 7.34-7.43 (m, 3 H) 7.67 (d, J=2.02 Hz, 1 H) 7.71-7.76 (m, 2 H) 8.34 (s, 1 H) 9.32 (s, 1 H).

### Example 46

### 4-(3-Chloro-4-fluoro-phenylamino)-6-[(D-pyrrolidin-2-ylmethyl)-amino]-quinoline-3-carbonitrile

4-(3-chloro-4-fluoro-phenylamino)-6-[(D-pyrrolidin-2-ylmethyl)-amino]-quinoline-3-carbonitrile was prepared according to the procedures described in Examples 40 and 43 above, yielding 77 mg of the desired product in 20% yield: 1H NMR (400 MHz, DMSO-D6) δ ppm 1.38-1.48 (m, 1 H) 1.62-1.79 (m, 2 H) 1.83-1.94 (m, 1 H) 2.81-2.94 (m, 2 H) 3.09-3.18 (m, 2 H) 3.26-3.32 (m, 1 H) 3.33-3.41 (m, 1 H) 6.41 (t, J=4.80 Hz, 1 H) 7.09 (d, J=2.27 Hz, 1 H) 7.21-7.27 (m, 1 H) 7.32 (dd, J=9.09, 2.27 Hz, 1 H) 7.39-7.48 (m, 2 H) 7.67 (d, J=9.09 Hz, 1 H) 8.30 (s, 1 H).

### Example 47

### 4-(3-Chloro-4-fluoro-phenylamino)-6-[(3H-imidazol-4-ylmethyl)-amino]-8-methoxy-quinoline-3-carbonitrile

Following the procedure described above in Example 4, 6-amino-4-(3-chloro-4-fluoro-phenylamino)-8-methoxy-quinoline-3-carbonitrile (190 mg, 0.55 mmol) was reacted with 4(5)-imidazole carboxaldehyde (53 mg, 0.55 mmol) and NaCNBH₃ (24 mg, 0.39 mmol) in 8 mL EtOH. The crude product was purified by preparative HPLC, and lyophilized to give the product as a solid (33 mg, 14%): 1 H NMR (400 MHz, DMSO-D6) δ ppm 3.85 (s, 3 H) 4.22-4.27 (m, J=4.29 Hz, 2 H) 6.34-6.41 (m, 1 H) 6.77 (d, J=1.77 Hz, 1 H) 6.92 (s, 1 H) 7.05 (s, 1 H) 7.18-7.24 (m, 1 H) 7.38-7.45 (m, 2 H) 7.61 (d, J=1.01 Hz, 1 H) 8.24 (s, 1 H) 9.21 (s, 1 H).

### Example 48

### 6-[5-Bromo-2-(2-methoxy-ethoxy)-benzylamino]-4-(3-chloro-4-fluoro-phenylamino)-quinoline-3-carbonitrile

Step 1: 5-Bromosalicylaldehyde (1 g, 5 mmol) was converted to 5-bromo-2-(2-methoxy-ethoxy)-benzaldehyde according to the procedure described above in Example 26 to obtain the desired product (657 mg) in 50% yield.

Step 2: Following the procedure described above in Example 4, 6-amino-4-(3-chloro-4-fluoro-phenylamino)-quinoline-3-carbonitrile (300 mg, 0.96 mmol) was reacted with 5-bromo-2-(2-methoxy-ethoxy)-benzaldehyde (248 mg, 0.96 mmol) and NaCNBH₃ (42 mg, 0.67 mmol) in 10 mL EtOH. The crude product was purified by preparative HPLC, and lyophilized to give the product as a solid (247 mg, 50%): 1 H NMR (400 MHz, DMSO-D6) 8 ppm 3.29 (s, 3 H) 3.64-3.69 (m, 2 H) 4.12-4.16 (m, 2 H) 4.36 (d, J=5.81 Hz, 2 H) 6.67 (t, J=5.94 Hz, 1 H) 6.98-7.01 (m, 1 H) 7.09 (d, J=2.53 Hz, 1 H) 7.14-7.19 (m, 1 H) 7.32-7.43 (m, 5 H) 7.72 (d, J=9.09 Hz, 1 H) 8.34 (s, 1 H) 9.31 (s, 1 H).

### Example 49

### 4-(3-Chloro-4-fluoro-phenylamino)-6-[(4,5,6,7-tetrafluoro-1H-indol-3-ylmethyl)-amino]-quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 4,5,6,7-Tetrafluoro-1H-indole-3-carbaldehyde (56 mg, 0.26 mmol) and NaCNBH₃ (12 mg, 0.18 mmol) in 5 mL EtOH. The crude product was purified by preparative HPLC, and lyophilized to give the product as a solid (76 mg, 57%): 1H NMR (400 MHz, DMSO-D6) δ ppm 4.50 (d, J=4.55 Hz, 2 H) 6.64 (t, J=5.18 Hz, 1 H) 7.16-7.24 (m, 2 H) 7.34-7.42 (m, 2 H) 7.44 (dd, J=6.06, 2.78 Hz, 1 H) 7.56 (d, J=1.77 Hz, 1 H) 7.69 (d, J=9.09 Hz, 1 H) 8.33 (s, 1 H) 9.34 (s, 1 H).

### Example 50

### 4-(3-Chloro-4-fluoro-phenylamino)-6-(4-methanesulfonyl-benzylamino)-quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 4-methylsulfonyl benzaldehyde (47 mg, 0.26 mmol) and NaCNBH₃ (12 mg, 0.18 mmol) in 5 mL EtOH. The crude product was purified by preparative HPLC, and lyophilized to give the product as a solid (33 mg, 26%): 1H NMR (400 MHz, DMSO-D6) δ ppm 3.18 (s, 3 H) 4.52 (d, J=5.56 Hz, 2 H) 7.01 (t, J=5.43 Hz, 1 H) 7.13-7.15 (m, 1 H) 7.18-7.23 (m, 1 H) 7.33-7.45 (m, 3 H) 7.62 (d, J=8.34 Hz, 2 H) 7.72 (d, J=9.35 Hz, 1 H) 7.89 (d, J=8.34 Hz, 2 H) 8.32 (s, 1 H) 9.29 (s, 1 H).

### Example 51

### 4-(3-Chloro-4-fluoro-phenylamino)-6-[(2-methoxy-pyridin-3-ylmethyl)-amino]-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 2-methoxy-pyridine-3-carbaldehyde (88 mg, 0.64 mmol) and NaCNBH₃ (28 mg, 0.45 mmol) in 5 mL EtOH. The crude product was purified by preparative HPLC, and lyophilized to give the product as a solid (108 mg, 55%): 1H NMR (400 MHz, DMSO-D6) δ ppm 3.91 (s, 3 H) 4.33 (d, J=5.81 Hz, 2 H) 6.74 (t, J=5.81 Hz, 1 H) 6.94 (dd, J=7.20, 5.18 Hz, 1 H) 7.06 (d, J=2.02 Hz, 1 H) 7.14-7.20 (m, 1 H) 7.33-7.43 (m, 3 H) 7.59 (d, J=6.82 Hz, 1 H) 7.72 (d, J=8.84 Hz, 1 H) 8.06 (d, J=5.05 Hz, 1 H) 8.33 (s, 1 H) 9.29 (s, 1 H).

### Example 52

### 3-{[4-(3-Chloro-4-fluoro-phenylamino)-3-cyano-quinolin-6-ylamino]-methyl}-pyrrolidine-1-carboxylic acid tert-butyl ester

Following the procedure described above in Example 4, 6-amino-4-(3-chloro-4-fluoro-phenylamino)-quinoline-3-carbonitrile (200 mg, 0.64 mmol) was reacted with 3-formyl-pyrrolidine-1-carboxylic acid tert-butyl ester (128 mg, 0.64 mmol) and NaCNBH₃ (28 mg, 0.45 mmol) in 5 mL EtOH. The crude product was purified by preparative HPLC, and lyophilized to give the product as a solid (168 mg, 75%): 1H NMR (400 MHz, DMSO-D6) 6 ppm 1.39 (s, 9 H) 1.58-1.72 (m, 1 H) 1.94-2.06 (m, 1 H) 2.42-2.48 (m, 1 H) 3.00 (d, J=10.48, 7.20 Hz, 1 H) 3.09-3.15 (m, 2 H) 3.18-3.29 (m, 1 H) 3.31-3.39 (m, 1 H) 3.45-3.52 (m, 1 H) 6.47 (t, J=5.43 Hz, 1 H) 7.01 (s, 1 H) 7.21-7.27 (m, 1 H) 7.30 (dd, J=9.09, 2.27 Hz, 1 H) 7.39-7.48 (m, 2 H) 7.68 (d, J=9.09 Hz, 1 H) 8.30 (s, 1 H) 9.31 (s, 1 H).

### Example 53

### 4-(3-Chloro-4-fluoro-phenylamino)-6-(3-hydroxy-benzylamino)-quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 3-hydroxy-benzaldehyde (31 mg, 0.26 mmol) and NaCNBH₃ (12 mg, 0.18 mmol) in 5 mL EtOH. The crude product was purified by preparative HPLC, and lyophilized to give the product as a solid (40 mg, 37%): 1H NMR (400 MHz, DMSO-D6) δ ppm 4.30 (d, J=5.81 Hz, 2 H) 6.61-6.66 (m, 1 H) 6.75-6.83 (m, 3 H) 7.08-7.15 (m, 2 H) 7.17-7.23 (m, 1 H) 7.33-7.45 (m, 3 H) 7.69 (d, J=8.84 Hz, 1 H) 8.31 (s, 1 H) 9.29-9.39 (m, 2 H).

### Example 54

### 4-(3-Chloro-4-fluoro-phenylamino)-6-(3-methyl-benzylamino)-quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with m-tolualdehyde (31 mg, 0.26 mmol) and NaCNBH₃ (12 mg, 0.18 mmol) in 5 mL EtOH. The crude product was purified by preparative HPLC, and lyophilized to give the product as a solid (48 mg, 45%): 1H NMR (400 MHz, DMSO-D6) δ ppm 2.27 (s, 3 H) 4.32 (d, J=5.81 Hz, 2 H) 6.82 (t, J=5.94 Hz, 1 H) 7.06 (d, J=7.07 Hz, 1 H) 7.09-7.23 (m, 5 H) 7.33-7.43 (m, 3 H) 7.69 (d, J=9.09 Hz, 1 H) 8.32 (s, 1 H) 9.30 (s, 1 H).

### Example 55

### 4-(3-Chloro-4-fluoro-phenylamino)-6-(2-hydroxy-benzylamino)-quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 2-hydroxy-benzaldehyde (S-265-2) (31 mg, 0.26 mmol) and NaCNBH₃ (12 mg, 0.18 mmol) in 5 mL EtOH. The crude product was purified by preparative HPLC, and lyophilized to give the product as a yellow solid (46 mg, 43%): 1H NMR (400 MHz, DMSO-D6) δ ppm 4.31 (d, J=5.56 Hz, 2 H) 6.54 (t, J=5.05 Hz, 1 H) 6.74 (t, J=7.33 Hz, 1 H) 6.84 (d, J=8.34 Hz, 1 H) 7.08 (t, J=8.21 Hz, 1 H) 7.15 (d, J=2.02 Hz, 1 H) 7.21 (d, J=6.57 Hz, 2 H) 7.35-7.45 (m, 3 H) 7.68 (d, J=8.84 Hz, 1 H) 8.30 (s, 1 H) 9.34 (s, 1 H) 9.58 (s, 1 H).

### Example 56

### 6-(2-Bromo-4-dimethylamino-benzylamino)-4-(3-chloro-4-fluoro-phenylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 2-bromo-4-dimethylamino-benzaldehyde (CL-242839-0) (146 mg, 0.64 mmol) and NaCNBH₃ (28 mg, 0.45 mmol) in 5 mL EtOH. The crude product was purified by preparative HPLC, and lyophilized to give the product as a yellow solid (139 mg, 40%): 1H NMR (400 MHz, DMSO-D6) δ ppm 2.88 (s,6 H) 4.26 (d, J=5.05 Hz, 2 H) 6.62 (t, J=5.05 Hz, 1 H) 6.69 (dd, J=8.59, 2.02 Hz, 1 H) 6.89 (d, J=2.78 Hz, 1 H) 7.08 (d, J=1.01 Hz, 1 H) 7.14-7.21 (m, 1 H) 7.24 (d, J=8.59 Hz, 1 H) 7.33-7.44 (m, 3 H) 7.70 (d, J=9.09 Hz, 1 H) 8.34 (s, 1 H) 9.31 (s, 1 H).

### Example 57

### 4-(3-Chloro-4-fluoro-phenylamino)-6-[5-cyano-2-(2-methoxy-ethoxy)-benzylamino]-quinoline-3-carbonitrile

6-[5-bromo-2-(2-methoxy-ethoxy)-benzylamino]-4-(3-chloro-4-fluoro-phenylamino)-quinoline-3-carbonitrile (150 mg, 0.27 mmol, prepared according to Example 48) was converted to 4-(3-Chloro-4-fluoro-phenylamino)-6-[5-cyano-2-(2-methoxy-ethoxy)-benzylamino]-quinoline-3-carbonitrile according to the procedure described above in Example 25 to obtain the desired compound (20 mg) in 15% yield. NMR: 1H NMR (400 MHz, DMSO-D6) δ ppm 3.29 (s, 3 H) 3.67-3.71 (m, 2 H) 4.23-4.28 (m, 2 H) 4.37 (d, J=5.81 Hz, 2 H) 6.69 (t, J=5.68 Hz, 1 H) 7.05 (d, J=2.53 Hz, 1 H) 7.13-7.18 (m, 1 H) 7.21 (d, J=8.59 Hz, 1 H) 7.32-7.40 (m, 3 H) 7.61 (d, J=2.02 Hz, 1 H) 7.71-7.76 (m, 2 H) 8.35 (s, 1 H) 9.30 (s, 1 H).

### Example 58

### 6-[2-Bromo-5-(2-ethoxy-ethoxy)-benzylamino]-4-(3-chloro-4-fluoro-phenylamino)-quinoline-3-carbonitrile

Following the procedure described above in Example 4, 6-amino-4-(3-chloro-4-fluoro-phenylamino)-quinoline-3-carbonitrile (200 mg, 0.64 mmol) was reacted with 2-bromo-5-(2-ethoxy-ethoxy)-benzaldehyde (WY-15245-1) (175 mg, 0.64 mmol) and NaCNBH₃ (28 mg, 0.45 mmol) in 5 mL EtOH. The crude product was purified by preparative HPLC, and lyophilized to give the product as a solid (145 mg, 40%): 1H NMR (400 MHz, DMSO-D6) δ ppm 1.06 (q, J=6.95 Hz, 3 H) 3.42 (q, J=7.07 Hz, 2 H) 3.57-3.62 (m, 2 H) 3.97-4.02 (m, 2 H) 4.35 (d, J=5.81 Hz, 2 H) 6.80-6.86 (m, 2 H) 7.02 (d, J=3.03 Hz, 1 H) 7.06 (d, J=2.02 Hz, 1 H) 7.12-7.18 (m, 1 H) 7.32-7.42 (m, 3 H) 7.50 (d, J=8.59 Hz, 1 H) 7.73 (d, J=9.09 Hz, 1 H) 8.35 (s, 1 H) 9.32 (s, 1 H).

### Example 59

### 4-(3-Chloro-4-fluoro-phenylamino)-6-(2-cyano-4-dimethylamino-benzylamino)-quinoline-3-carbonitrile

6-(2-bromo-4-dimethylamino-benzylamino)-4-(3-chloro-4-fluoro-phenylamino)-quinoline-3-carbonitrile (100 mg, 0.19 mmol, prepared according to the procedure described in Example 56) was converted to 4-(3-Chloro-4-fluoro-phenylamino)-6-(2-cyano-4-dimethylamino-benzylamino)-quinoline-3-carbonitrile according to the procedure described above in Example 25 to obtain the desired compound (20 mg) in 21 % yield: 1H NMR (400 MHz, DMSO-D6) δ ppm 2.99 (s, 6 H) 5.08 (s, 2 H) 7.07 (dd, J=8.59, 2.53 Hz, 1 H) 7.30-7.37 (m, 1 H) 7.44-7.50 (m, 3 H) 7.57 (dd, J=6.69, 2.65 Hz, 1 H) 8.01 (d, J=9.35 Hz, 1 H) 8.20 (s, 1 H) 8.43 (d, J=2.27 Hz, 1 H) 8.57 (s, 1 H) 9.03 (dd, J=9.60, 4.29 Hz, 1 H).

### Example 60

### 4-(3-Chloro-4-fluoro-phenylamino)-6-[2-cyano-5-(2-ethoxy-ethoxy)-benzylamino]-quinoline-3-carbonitrile

6-[2-bromo-5-(2-ethoxy-ethoxy)-benzylamino]-4-(3-chloro-4-fluoro-phenylamino)-quinoline-3-carbonitrile (100 mg, 0.1 8 mmol, prepared according to the procedure described in Example 56) was converted to 4-(3-Chloro-4-fluoro-phenylamino)-6-[2-cyano-5-(2-ethoxy-ethoxy)-benzylamino]-quinoline-3-carbonitrile according to the procedure described above in Example 25 to obtain the desired compound (16 mg) in 17 % yield: 1H NMR (400 MHz, DMSO-D6) 8 ppm 1.14 (t, J=6.95 Hz, 3 H) 3.52 (q, J=7.07 Hz, 2 H) 3.71-3.76 (m, 2 H) 4.17-4.23 (m, 2 H) 5.10 (s, 2 H) 7.13 (dd, J=8.34, 2.27 Hz, 1 H) 7.17 (d, J=2.27 Hz, 1 H) 7.28-7.36 (m, 1 H) 7.47 (t, J=8.97 Hz, 1 H) 7.56 (dd, J=6.44, 2.40 Hz, 1 H) 7.97 (d, J=9.35 Hz, 1 H) 8.01 (d, J=8.34 Hz, 1 H) 8.27 (s, 1 H) 8.35 (d, J=2.27 Hz, 1 H) 8.52 (s, 1 H) 9.20 (d, J=11.62 Hz, 1 H).

### Example 61

### 4-(3-Chloro-4-fluoro-phenylamino)-6-[(tetrahydro-pyran-4-ylmethyl)-amino]-quinoline-3-carbonitrile

Following the procedure described above in Example 4, 6-amino-4-(3-chloro-4-fluoro-phenylamino)-quinoline-3-carbonitrile (150 mg, 0.48 mmol) was reacted with tetrahydro-pyran-4-carbaldehyde (56 mg, 0.48 mmol) and NaCNBH₃ (21 mg, 0.34 mmol) in 10 mL EtOH. The crude product was purified by preparative HPLC, and lyophilized to give the product as a solid (1 44 mg, 73%): 1H NMR (400 MHz, DMSO-D6) δ ppm 1.18-1.32 (m, 2 H) 1.70 (d, J=13.14 Hz, 2 H) 1.75-1.88 (m, 1 H) 2.96-3.05 (m, 2 H) 3.24-3.30 (m, 2 H) 3.87 (dd, J=11.49, 2.91 Hz, 2 H) 6.40 (t, J=5.43 Hz, 1 H) 6.97 (d, J=2.02 Hz, 1 H) 7.19-7.27 (m, 1 H) 7.31 (dd, J=9.09, 2.27 Hz, 1 H) 7.39-7.48 (m, 2 H) 7.67 (d, J=9.09 Hz, 1 H) 8.33 (s, 1 H) 9.36 (s, 1 H).

### Example 62

### 4-(3-Chloro-4-fluoro-phenylamino)-6-[(pyrrolidin-3-ylmethyl)-amino]-quinoline-3-carbonitrile

3-{[4-(3-Chloro-4-fluoro-phenylamino)-3-cyano-quinolin-6-ylamino]-methyl}-pyrrolidine-1-carboxylic acid tert-butyl ester (80 mg, 0.16 mmol, prepared according to Example 52) was converted to 4-(3-Chloro-4-fluoro-phenylamino)-6-[(pyrrolidin-3-ylmethyl)-amino]-quinoline-3-carbonitrile following the procedure described in Example 43 to obtain desired product (84 mg) in quantitative yield: 1H NMR (400 MHz, DMSO-D6) δ ppm 1.60-1.74 (m, 1 H) 2.05-2.22 (m, 1 H) 2.58-2.68 (m, 1 H) 2.83-2.95 (m, 1 H) 3.10-3.33 (m, 4 H) 3.34-3.45 (m, 1 H) 6.87 (s, 1 H) 7.25 (s, 1 H) 7.39-7.48 (m, 2 H) 7.54 (t, J=9.09 Hz, 1 H) 7.71 (dd, J=6.19, 2.15 Hz, 1 H) 7.76 (d, J=9.09 Hz, 1 H) 8.67 (s, 1 H) 8.84 (s, 2 H).

### Example 63

### 3-{[4-(3-Chloro-4-fluoro-phenylamino)-3-cyano-quinolin-6-ylamino]-methyl}-piperidine-1-carboxylic acid tert-butyl ester

Following the procedure described above in Example 4, 6-amino-4-(3-chloro-4-fluoro-phenylamino)-quinoline-3-carbonitrile (200 mg, 0.64 mmol) was reacted with 3-formyl-piperidine-1-carboxylic acid tert-butyl ester (136 mg, 0.64 mmol) and NaCNBH₃ (28 mg, 0.45 mmol) in 5 mL EtOH. The crude product was purified by preparative HPLC, and lyophilized to give the product as a solid (51 mg, 16%): 1H NMR (500 MHz, DMSO-D6) δ ppm 1.17-1.28 (m, 2 H) 1.30-1.44 (m, 10 H) 1.60-1.68 (m, 1 H) 1.70-1.80 (m, 1 H) 1.85 (d, J=13.12 Hz, 1 H) 2.62-2.71 (m, 1 H) 2.79-2.89 (m, 1 H) 3.76 (d, J=12.82 Hz, 1 H) 3.93 (d, J=14.65 Hz, 1 H) 6.21-6.39 (m, 2 H) 6.98 (s, 1 H) 7.15-7.23 (m, 1 H) 7.30-7.35 (m, 1 H) 7.38 (t, J=8.85 Hz, 2 H) 7.70 (d, J=8.85 Hz, 1 H) 8.33 (s, 1 H) 9.18 (s, 1 H).

### Example 64

### 4-(3-Chloro-4-fluoro-phenylamino)-6-[(piperidin-3-ylmethyl)-amino]-quinoline-3-carbonitrile

3-{[4-(3-chloro-4-fluoro-phenylamino)-3-cyano-quinolin-6-ylamino]-methyl}-piperidine-1-carboxylic acid tert-butyl ester (70 mg, prepared according to the procedure described in Example 63) was converted to 4-(3-chloro-4-fluoro-phenylamino)-6-[(piperidin-3-ylmethyl)-amino]-quinoline-3-carbonitrile following the procedure described in Example 43 to obtain desired product (70 mg) in quantitative yield: 1H NMR (400 MHz, DMSO-D6) δ ppm 1.21-1.32 (m, 1 H) 1.53-1.67 (m, 1 H) 1.87 (dd, J=32.21, 13.77 Hz, 2 H) 1.98-2.13 (m, J=14.15 Hz, 1 H) 2.61-2.74 (m, 1 H) 2.74-2.86 (m, 1 H) 3.05-3.20 (m, 2 H) 3.26 (d, J=12.38 Hz, 1 H) 3.36 (d, J=12.88 Hz, 1 H) 6.79 (s,1 H) 7.21 (d, J=2.02 Hz, 1 H) 7.42 (dd, J=9.09, 2.02 Hz, 2 H) 7.54 (t, J=8.97 Hz, 1 H) 7.69 (dd, J=6.57, 2.27 Hz, 1 H) 7.75 (d, J=9.09 Hz, 1 H) 8.44 (d, J=11.12 Hz, 1 H) 8.65 (s, 1 H) 8.72 (d, J=11.12 Hz, 1 H).

### Example 65

### Methyl 2-(2-cyano-3-ethoxy-3-oxoprop-1-enylamino)-5-nitrobenzoate

Step 1: Following the procedure reported by J. Kerrigan and L. Vagnoni (Tetrahedron 2001, 57, 8227-8235) 5-nitroanthranilic acid (1.00 g, 5.49 mmol) was taken up in 10 mL MeOH and 5 mL benzene in a 100 mL 2-necked round-bottomed flask fitted with a condenser and Dean-Stark trap, and 0.7 mL concentrated sulfuric acid was added. The mixture was heated at reflex overnight, during which time most of the solvent distilled out of the flask. TLC analysis (100% EtOAc) indicated that the acid starting material had been completely consumed. The cooled reaction mixture was diluted with 10 mL MeOH and 40 mL EtOAc, washed 3 times with saturated NaHCO₃ and once with brine, dried over anhydrous MgSO₄, filtered, and evaporated to give pure product methyl 2-amino-5-nitrobenzoate as a yellow solid (0.89 g, 82% yield): ¹H NMR (400 MHz, DMSO-D₆) δ 3.86 (s, 3 H) 6.90 (d, J=9.4 Hz, 1 H) 7.84 (s, 2 H) 8.09 (dd, J=9.2, 2.9 Hz, 1 H) 8.59 (d, J=2.8 Hz, 1 H).

Step 2: In a 1 L round-bottomed flask, the product from the previous step (28.0 g, 0.143 mol) was taken up in 140 mL DMF, and ethyl (ethoxymethylene)cyanoacetate (26.6 g, 0.157mol) was added. The mixture was stirred vigorously until both reagents went into solution, and Cs₂CO₃ (93 g, 0.29mol) was added. The flask was capped with a rubber septum and shaken by hand until the reaction mixture solidified after 5 minutes, turning a deep reddish-orange color. TLC analysis (40% EtOAc in hexanes) showed complete consumption of the aniline starting material. The slurry was poured into 16OOmL 1:1 EtOAc/water and stirred vigorously, and the pale yellow precipitate collected by suction filtration, washed 3 times with water, and dried under vacuum for 2 days. Pure product was obtained as a yellow solid (39 g, 87% yield): ¹H NMR (400 MHz, DMSO-D₆) 6 1.28 (t, J=7.1 Hz, 3 H) 3.96 (s, 3 H) 4.28 (q, J=7.2 Hz, 2 H) 8.11 (d, J=9.4 Hz, 1 H) 8.45 (dd, J=9.4, 2.8 Hz, 1 H) 8.70 (d, J=2.8 Hz, 1 H) 8.79 (d, J=13.1 Hz, 1 H) 12.80 (d, J=12.9 Hz, 1 H); HRMS (ESI+) calcd for C₁₄H₁₆N₄O₆ (M[+NH₃]+) 337.1142, found 337.1144.

### Example 66

### 6-(benzylamino)-4-(4-morpholinophenylamino)quinoline-3-carbonitrile

Step 1: In a 1OOmL round-bottomed flask fitted with a condenser, 6-iodo-4-oxo-1,4-dihydroquinoline-3-carbonitrile (1.00 g, 3.38 mmol) was taken up in 12 mL POCl₃ and heated at reflux for 1 hour. The reaction mixture was then allowed to cool to RT, and the POCl₃ removed under reduced pressure. The residue was partitioned between 6OmL each of CH₂Cl₂ and 5% Na₂CO₃; a scoopful of solid Na₂CO₃ was added, and the mixture stirred for 30 minutes, checking the pH periodically to ensure that it remained at or above 8. The layers were then separated, and the aqueous layer extracted with additional CH₂Cl₂. The combined organic layers were filtered through Celite and evaporated to give pure product 4-chloro-6-iodoquinoline-3-carbonitrile (0.93 g, 88% yield): ¹H NMR (400 MHz, DMSO-D₆) δ 7.94 (d, J=8.8 Hz, 1 H) 8.32 (dd, J=8.4, 1.8 Hz, 1 H) 8.59-8.68 (m, 1 H) 9.21 (s, 1 H).

Step 2: In a 300 mL round-bottomed flask equipped with a condenser, the product from the previous step (0.93 g, 3.0 mmol) was taken up in 40 mL 2-ethoxyethanol, and 4-morpholinoaniline (0.58 g, 3.3 mmol) in 40 mL 2-ethoxyethanol was added in one portion. The reaction mixture was heated at reflux for 1 hour, until TLC analysis (20% EtOAc in hexanes) showed complete disappearance of the 4-chloro-6-iodoquinoline-3-carbonitrile. The reaction mixture was then allowed to cool to RT, 80 mL each EtOAc and 5% Na₂CO₃ were added, and the suspension allowed to stir for 30 minutes. The bright yellow precipitate was collected by suction filtration, washed with water, and dried under vacuum to give pure product 6-iodo-4-(4-morpholinophenylamino)quinoline-3-carbonitrile (1.09 g, 81 % yield): ¹H NMR (400 MHz, DMSO-D₆) δ 3.03-3.22 (m, 4 H) 3.69-3.83 (m, 4 H) 6.97 (d, J=9.1 Hz, 2 H) 7.19 (d, J=9.1 Hz, 2 H) 7.64 (d, J=8.6 Hz, 1 H) 8.06 (dd, J=8.7, 1.9 Hz, 1 H) 8.47 (s, 1 H) 8.92 (d, J=1.8 Hz, 1 H) 9.75 (s, 1 H).

Step 3: Following the procedure reported by F. Kwong, A. Klapars and S. Buchwald (Org. Lett. 2002, 4(4), 581-584), the product from step 2 (0.20 g, 0.438 mmol), Cul (16.8 mg, 0.088 mmol) and freshly ground K₃PO₄ (186 mg, 0.88 mmol) were placed in a test tube fitted with an aluminum crimp seal. The tube was sealed, and a solution of benzylamine (0.11 4 mL, 11 2 mg, 1.0 mmol) and ethylene glycol (0.048 mL, 54 mg, 0.876 mmol) in isopropanol was added via syringe. The tube was heated in an oil bath at 90° C. for 2 days, until TLC analysis showed significant conversion of 6-iodoquinoline to product. The reaction mixture was then cooled to RT and partitioned between EtOAc and brine. The aqueous layer was extracted 3 times with additional EtOAc, and the combined organic layers washed with brine, dried over anhydrous MgSO₄, filtered, and evaporated. The crude product was purified by flash chromatography over silica gel (40% EtOAc in CH₂Cl₂) and lyophilized to give the product as a fluffy, bright yellow solid (9.3 mg, 2.1% yield): ¹H NMR (400 MHz, DMSO-D₆) δ 3.06-3.15 (m, 4 H) 3.70-3.78 (m, 4 H) 4.38 (d, J=5.8 Hz, 2 H) 6.71 (t, J=6.1 Hz, 1 H) 6.96 (d, J=9.1 Hz, 2 H) 7.12 (d, J=9.1 Hz, 2 H) 7.22-7.41 (m, 7 H) 7.61 (d, J=8.8 Hz, 1 H) 8.16 (s, 1 H) 9.15 (s, 1 H); HRMS (ESI+) calcd for C₂₇H₂₆N₅O (MH+) 436.2132, found 436.2130.

### Example 67

### 6-bromo-4-(4-methoxyphenylamino)quinoline-3-carbonitrile

Step 1: Following the procedure described above in Example 66, 6-bromo-4-oxo-1,4-dihydroquinoline-3-carbonitrile (1.00 g, 4.02 mmol) was converted to 6-bromo-4-chloroquinoline-3-carbonitrile in quantitative yield (1.07 g, 100% yield): ¹H NMR (400 MHz, DMSO-D₆) δ 8.09-8.14 (m, 1 H) 8.16-8.21 (m, 1 H) 8.46 (dd, J=2.0, 0.5 Hz, 1 H) 9.23 (s, 1 H).

Step 2: Following the procedure described above in Example 66, 6-bromo-4-chloroquinoline-3-carbonitrile (1.08 g, 4.04 mmol) was reacted with p-anisidine (0.547 g, 4.44 mmol) in 55 mL 2-ethoxyethanol. Work-up of the cooled reaction mixture gave a brown oil which solidified under vacuum. This was washed twice with hexanes and dried under vacuum to give a beige-colored crystalline material (1:1 complex with 2-ethoxyethanol, 1.8 g, 53% yield): ¹H NMR (400 MHz, DMSO-D₆) δ 1.09 (t, J=7.0 Hz, 3 H) 3.36 (t, J=5.2 Hz, 2 H) 3.41 (q, J=7.0 Hz, 2 H) 3.47 (q, J=5.4 Hz, 2 H) 3.78 (s, 3 H) 4.55 (t, J=5.6 Hz, 1 H) 6.98 (d, J=8.8 Hz, 2 H) 7.27 (d, J=8.8 Hz, 2 H) 7.82 (d, J=9.1 Hz, 1 H) 7.95 (dd, J=9.0, 2.2 Hz, 1 H) 8.50 (s, 1 H) 8.79 (d, J=2.0 Hz, 1 H) 9.81 (s, 1 H); HRMS (ESI+) calcd for C₁₇H₁₃BrN₃O (MH+) 354.0237, found 354.0238.

### Example 68

### 4-(3-chlorophenylamino)-3-cyano-N,N-dimethyl-6-(2-morpholinoethylamino)quinoline-8-carboxamide

Step 1: In a 2 L round-bottomed flask, 5-nitroanthranilic acid (100 g, 0.55mol) and dimethylamine hydrochloride (50 g, 0.60mol) were taken up in 500 mL DMF. Once both reagents had dissolved, benzotriazol-1-yloxytris(dimethylamino)phosphonium hexafluorophosphate (268 g, 0.604mol) was added, followed by 4-methylmorpholine (134 mL, 122 g, 1.21 mol). The mixture was stirred at RT overnight, then poured into 5 L water and stirred vigorously until the suspension was evenly mixed. The precipitate was collected by suction filtration, washed with water three times, and dried under vacuum to give pure product 2-amino-N,N-dimethyl-5-nitrobenzamide as a yellow powder (103 g, 89% yield): ¹H NMR (400 MHz, DMSO-D₆) δ 2.93 (br s, 6 H) 6.69 (s, 2 H) 6.75 (d, J=9.4 Hz, 1 H) 7.88 (d, J=2.8 Hz, 1 H) 7.98 (dd, J=9.1, 2.8 Hz, 1 H).

Step 2: In a 2 L round-bottomed flask, the product from the previous step (116 g, 0.554 mol) and ethyl (ethoxymethylene)cyanoacetate (1 88 g, 1.11 mol) were dissolved in 580 mL DMF, and Cs₂CO₃ (362 g, 1.11 mol) was added. The mixture was heated to 45° C. for 2 hours, then cooled to RT, stirred overnight, and poured into 5 L water. The yellow precipitate was collected by suction filtration, washed three times with water, and dried under vacuum to give pure product ethyl 2-cyano-3-(2-(dimethylcarbamoyl)-4-nitrophenylamino)acrylate (174 g, 94% yield): ¹H NMR (400 MHz, DMSO-D₆) δ 1.28 (t, J=7.1 Hz, 3 H) 2.94 (s, 3 H) 3.06 (s, 3 H) 4.26 (q, J=7.2 Hz, 2 H) 8.00 (d, J=9.4 Hz, 1 H) 8.27 (d, J=2.5 Hz, 1 H) 8.30-8.37 (m, 1 H) 8.72 (d, J=12.9 Hz, 1 H) 11.34 (d, J=13.4 Hz, 1 H).

Step 3: In each of two 3 L 3-necked round-bottomed flasks fitted with stir bars, ethylene glycol/water cooled condensers, heating mantles, inert gas inlets/outlets and an internal device to monitor reaction temperature, the product from step 2 (26.9 g, 80.9 mmol) was suspended in 1.5 L Dowtherm A. Argon or nitrogen was bubbled through each suspension by means of a long needle for 40 minutes. The two flasks were then heated to 260° C. overnight, with inert gas being continually passed through. They were then allowed to cool to RT. The contents of each flask were poured into 2.4 L hexanes, stirred vigorously and filtered, and the brown precipitate was washed twice with hexanes and once with EtOH and dried under vacuum overnight. This gave a brown powder of sufficient purity to be used in the next step (28 g, 61% yield): ¹H NMR (400 MHz, DMSO-D₆) δ 2.86 (s, 3 H) 3.09 (s, 3 H) 8.48 (d, J=2.5 Hz, 1 H) 8.70 (s, 1 H) 8.85 (d, J=2.5 Hz, 1 H) 12.61 (br s, 1 H).

Step 4: In a 1 L round-bottomed flask fitted with an addition funnel, 3-cyano-N,N-dimethyl-6-nitro-4-oxo-1,4-dihydroquinoline-8-carboxamide (28 g, 98 mmol) was suspended in 200 mL DCE, and 1 mL DMF was added. Oxalyl chloride (17 mL, 25 g, 0.20mol) was then added dropwise via the addition funnel. After the addition was complete, the addition funnel was replaced with a reflux condenser, and the mixture was refluxed for 2 hours. It was then allowed to cool to RT, and the solvent and excess oxalyl chloride removed under reduced pressure. The residue was taken up in CHCl₃ and passed over a short column of silica gel in a Buchner funnel, eluting with additional CHCl₃. Evaporation of the solvent gave product 4-chloro-3-cyano-N,N-dimethyl-6-nitroquinoline-8-carboxamide as a brown powder (13.8 g, 46% yield): ¹H NMR (400 MHz, DMSO-D₆) δ 2.69 (s, 3 H) 3.11 (s, 3 H) 8.64 (d, J=2.3 Hz, 1 H) 9.06 (d, J=2.5 Hz, 1 H) 9.43 (s, 1 H).

Step 5: In a microwave vial, 4-chloro-3-cyano-N,N-dimethyl-6-nitroquinoline-8-carboxamide (1.33 g, 4.37 mmol) and 3-chloroaniline (0.50 mL, 0.61 g, 4.8 mmol) were taken up in 5 mL DME. The vial was crimp-sealed and heated in a microwave reactor at 140° C. for 10 minutes. The contents of the vial were transferred to a separatory funnel and partitioned between EtOAc and 5% Na₂CO₃, and the aqueous layer extracted two additional times with EtOAc. The combined organic extracts were washed with brine, dried over anhydrous MgSO₄, filtered and evaporated to give a solid. The crude product was purified by flash chromatography over silica gel (30-50% EtOAc in CH₂Cl₂) to give product 4-(3-chlorophenylamino)-3-cyano-N,N-dimethyl-6-nitroquinoline-8-carboxamide as a yellow solid (0.74 g, 43% yield): ¹H NMR (400 MHz, DMSO-D₆) δ 2.71 (s, 3 H) 3.09 (s, 3 H) 7.33-7.40 (m, 2 H) 7.47 (d, J=7.8 Hz, 1 H) 7.49-7.51 (m, 1 H) 8.44 (d, J=2.3 Hz, 1 H) 8.83 (s, 1 H) 9.58 (d, J=2.5 Hz, 1 H) 10.62 (s, 1 H).

Step 6: In a 250 mL 2-necked round-bottomed flask fitted with a condenser, the product from step 5 (0.74 g, 1.9 mmol) was taken up in 30 mL EtOH and tin chloride dihydrate (2.11 g, 9.35 mmol) was added. The reaction mixture was heated at reflux for 2 hours, until TLC analysis showed complete disappearance of the nitroquinoline. The reaction mixture was then cooled to RT and poured into ice water. The orange suspension was neutralized with saturated NaHCO₃ and extracted into CHCl₃ (3 times), and the combined organic layers washed with brine, dried over anhydrous MgSO₄, filtered and evaporated. Evaporation of the CHCl₃ extracts gave 6-amino-4-(3-chlorophenylamino)-3-cyano-N,N-dimethylquinoline-8-carboxamide as a yellow powder, 0.35 g, 51 % yield): ¹H NMR (400 MHz, DMSO-D₆) δ 2.69 (s, 3 H) 3.05 (s, 3 H) 5.91 (s, 2 H) 7.03-7.08 (m, 1 H) 7.08-7.12 (m, 1 H) 7.11 (d, J=2.3 Hz, 1 H) 7.17 (t, J=2.0 Hz, 2 H) 7.33 (t, J=8.0 Hz, 1 H) 8.41 (s, 1 H) 9.44 (s, 1 H); HRMS (ESI+) calcd for C₁₉H₁₇ClN₅0 (MH+) 366.1116, found 366.1118.

Step 7: Following the procedure described above in Example 4, 6-amino-4-(3-chlorophenylamino)-3-cyano-N,N-dimethylquinoline-8-carboxamide (0.29 g, 0.79 mmol) was reacted with morpholin-4-yl-acetaldehyde (prepared by heating the corresponding dimethyl acetal (1.037 g, 5.98 mmol) in 2.0 mL concentrated HCl and 1.5 mL water for 6 hours in a microwave reactor at 70° C., then carefully neutralizing with solid NaHCO₃ until pH=6) and NaCNBH₃ (110 mg, 1.76 mmol) in 12 mL EtOH. The crude product was purified by flash chromatography over silica gel (6% MeOH in CH₂Cl₂) and lyophilized, giving pure product as a fluffy, bright yellow solid (48 mg, 13% yield): ¹H NMR (400 MHz, DMSO-D₆) δ 2.42 (br s, 4 H) 2.54 (t, J=6.7 Hz, 2 H) 2.68 (s, 3 H) 3.04 (s, 3 H) 3.25 (q, J6.3 Hz, 2 H) 3.33 (s, 4 H) 3.48-3.64 (m, 4 H) 6.31 (t, J=5.3 Hz, 1 H) 7.07 (d, J=2.3 Hz, 1 H) 7.10-7.22 (m, 3 H) 7.25 (t, J=2.0 Hz, 1 H) 7.38 (t, J=8.0 Hz, 1 H) 8.38 (s, 1 H) 9.41 (s, 1 H); HRMS (ESI+) calcd for C₂₅H₂₈ClN₆O₂ (MH+) 479.1957, found 479.1975.

### Example 69

### 8-bromo-4-(3-chlorophenylamino)-6-(2-morpholinoethylamino)quinoline-3-carbonitrile

Step 1: According to the procedure described by M. Kotharé et al. (Tetrahedron, 2000, 56, 9833-9841), 4-nitroaniline (50 g, 0.36mol) was suspended in 465 mL glacial acetic acid in a 2 L Erlenmeyer flask. A solution of bromine (19 mL, 58 g, 0.36mol) in 280 mL acetic acid was added from an addition funnel, with stirring. After addition was complete, the reaction mixture was allowed to stir for 1 hour, then warmed to 60° C. and poured into 1.1 L ice water. The precipitate, a slightly dirty bright yellow color, was collected by suction filtration. It was then taken up in 1 L Et₂O, washed twice with saturated NaHCO₃, dried over anhydrous MgSO₄, filtered and evaporated to give pure product 2-bromo-4-nitroaniline as a bright yellow powder (64 g, 82% yield): ¹H NMR (400 MHz, DMSO-D₆) δ 6.82 (m, 3 H) 7.97 (dd, J=9.1, 2.5 Hz, 1 H) 8.22 (d, J=2.8 Hz, 1 H).

Step 2: A 2 L round-bottomed flask was charged with 2-bromo-4-nitroaniline (71 g, 0.33 mol), ethyl (ethoxymethylene)cyanoacetate (111 g, 0.654 mol) and 355 mL DMF. The mixture was stirred vigorously to dissolve both reagents, Cs₂CO₃ (213 g, 0.654 mol) was added, and the reaction mixture was allowed to stir overnight. To work up, the contents of the flask were poured into 2.5 L water and the precipitate collected by suction filtration. The filter cake was then re-suspended in water, stirred, and collected again. This was done three times, and the product was then allowed to dry on the Buchner funnel overnight under suction. It was then washed three times with Et₂O and three times with hexanes, each time suspending the filter cake in the solvent of choice, stirring vigorously for 5-20 minutes, and re-filtering. Finally, the product ethyl 3-(2-bromo-4-nitrophenylamino)-2-cyanoacrylate was dried overnight under vacuum to give pure material as a free-flowing, pale yellow powder (WAY-191748, 99 g, 90% yield): ¹H NMR (400 MHz, DMSO-D₆) δ 1.29 (t, J=7.1 Hz, 3 H) 4.30 (q, J=7.1 Hz, 2 H) 8.02 (d, J=9.4 Hz, 1 H) 8.31 (dd, J=9.1, 2.5 Hz, 1 H) 8.56 (d, J=2.5 Hz, 1 H) 8.86 (d, J=12.6 Hz, 1 H) 11.31 (d, J=13.1 Hz, 1 H); HRMS (ESI+) calcd for C₁₂H₁₀N₃NaO₄ (MNa+) 361.9747, found 361.9742.

Step 3: Following the procedure described above in Example 68, two batches of ethyl 3-(2-bromo-4-nitrophenylamino)-2-cyanoacrylate (30.3 g each, 89.1 mmol) were cyclized. The product 8-bromo-6-nitro-4-oxo-1,4-dihydroquinoline-3-carbonitrile was obtained as a brown powder of sufficient purity to be used directly in the next step (WAY-191772, 42 g, 79% yield): ¹H NMR (400 MHz, DMSO-D₆) δ 8.69 (s, 1 H) 8.82 (dd, J=12.9, 2.5 Hz, 2 H) 12.55 (br s,1 H); HRMS (ESI+) calcd for C₁₀H₅BrN₃O₃ (MH+) 293.9510, found 293.9509.

Step 4: Following the procedure described above in Example 68, 8-bromo-6-nitro-4-oxo-1,4-dihydroquinoline-3-carbonitrile (1 8 g, 59 mmol) was reacted with oxalyl chloride (1OmL, 15 g, 0.12 mol) in 120 mL DCE, with 1 mL DMF. The product 8-bromo-4-chloro-6-nitroquinoline-3-carbonitrile was isolated as a brown powder (13.4 g, 72% yield): ¹H NMR (400 MHz, DMSO-D₆) δ 9.02-9.06 (m, 2 H) 9.52 (s, 1 H). Anal. Calcd for C₁₀H₃BrClN₃O₂: C, 38.43; H, 0.97; N, 13.45. Found: C, 38.11; H, 0.92; N, 13.05.

Step 5: According to the procedure described above in Example 68, 8-bromo-4-chloro-6-nitroquinoline-3-carbonitrile (1.00 g, 3.20 mmol) was reacted with 3-chloroaniline (0.37 mL, 0.45 g, 3.5 mmol) in 5 mL DME. The crude product was purified by flash chromatography over silica gel (5% EtOAc in CH₂Cl₂), to give pure product 8-bromo-4-(3-chlorophenylamino)-6-nitroquinoline-3-carbonitrile as a yellow solid (0.90 g, 70% yield): ¹H NMR (400 MHz, DMSO-D₆) 6 7.33-7.41 (m, 2 H) 7.45-7.51 (m, 2 H) 8.88 (d, J=2.3 Hz, 1 H) 8.91 (s, 1 H) 9.57 (d, J=2.5 Hz, 1 H) 1 H); HRMS (ESI+) calcd for C₁₆H₉BrClN₄O₂ (MH+) 402.9592, found 402.9587. Anal. Calcd for C₁₆H₈BrClN₄O₂: C, 47.61; H, 2.00; N, 13.88. Found: C, 47.79; H, 1.85; N, 13.66.

Step 6: In a microwave vial, the product from step 5 (0.500 g, 1.24 mmol) was taken up in 5 mL EtOH and tin chloride dihydrate (1.40 g, 6.19 mmol) was added. The vial was sealed and heated in a microwave reactor at 110° C. for 5 minutes, until TLC analysis showed complete disappearance of the nitroquinoline. The contents of the vial were then emptied into ice water, and the reaction worked up as described above in Example 69 Step 5. Purification of the crude product by flash chromatography over silica gel (10-40% EtOAc in CH₂Cl₂) gave pure product 6-amino-8-bromo-4-(3-chlorophenylamino)quinoline-3-carbonitrile as a brownish-yellow solid 0.195 g, 42% yield): ¹H NMR (400 MHz, DMSO-D₆) δ 5.98 (s, 2 H) 7.03-7.08 (m, 1 H) 7.08-7.13 (m, 1 H) 7.17 (t, J=2.2 Hz, 2 H) 7.33 (t, J=8.1 Hz, 1 H) 7.66 (d, J=2.3 Hz, 1 H) 8.48 (s, 1 H) 9.48 (s, 1 H); HRMS (ESI+) calcd for C₁₆H₁₀BrClN₄ (MH+) 372.9850, found 372.9856.

Step 7: In a 1 L round-bottomed flask, 6-amino-8-bromo-4-(3-chlorophenylamino)quinoline-3-carbonitrile (3.31 g, 8.86 mmol) was taken up in 270 mL EtOH. 2-morpholinoacetaldehyde (prepared by heating the corresponding dimethyl acetal overnight in 21 mL concentrated HCl and 34 mL water, at 70° C., then neutralizing the solution with saturated NaHCO₃) was added and the mixture was stirred at RT for 2 hours, then refluxed for 1 hour. After cooling to RT, the solution was acidified to pH 4 with acetic acid, and NaCNBH₃ (0.61 g, 9.8 mmol) was added. The mixture was allowed to stir at RT overnight. Most of the solvent was then removed under reduced pressure, and the residue partitioned between EtOAc and enough 5% Na₂CO₃ to bring the aqueous layer to a neutral pH. The aqueous layer was extracted twice more with EtOAc, and the combined organic layers were washed with brine, dried over anhydrous MgSO₄, filtered, and evaporated. The crude product was purified twice by flash chromatography over silica gel (5% MeOH in CH₂Cl₂, then 3% MeOH in CH₂Cl₂), and lyophilized. Pure product was obtained as a fluffy golden-brown solid (0.506 g, 12% yield): ¹H NMR (400 MHz, DMSO-D₆) δ 2.41 (s, 4 H) 2.51-2.59 (m, 2 H) 3.14-3.27 (m, 2 H) 3.48-3.65 (m, 4 H) 6.38 (t, J=5.6 Hz, 1 H) 7.07 (d, J=2.3 Hz, 1 H) 7.11-7.16 (m, 1 H) 7.17-7.22 (m, 1 H) 7.25 (t, J=2.0 Hz, 1 H) 7.38 (t, J=8.1 Hz, 1 H) 7.77 (d, J=2.3 Hz, 1 H) 8.44 (s, 1 H) 9.44 (s, 1 H); HRMS (ESI+) calcd for C₂₂H₂₂BrClN₅O (MH+) 486.0691, found 486.0696.

### Example 70

### 8-bromo-4-(3-chlorophenylamino)-6-(pyridin-3-ylmethylamino)quinoline-3-carbonitrile

Following the procedure described above in Example 4, 6-amino-8-bromo-4-(3-chlorophenylamino)quinoline-3-carbonitrile (4.42 g, 11.8 mmol) was reacted with 3-pyridinecarboxaldehyde (1.1 mL, 1.2 g, 11 mmol) and NaCNBH₃ (0.48 g, 7.6 mmol) in 1800 mL EtOH. Ethanol was removed under reduced pressure, the residue partitioned between EtOAc and 5% Na₂CO₃, and the aqueous layer extracted with 4 additional portions of EtOAc. The combined organic layers were then washed with brine, evaporated, and purified by trituration with 150 mL boiling EtOH to give pure product as a yellowish-brown powder (2.51 g, 46% yield): ¹H NMR (400 MHz, DMSO-D₆) 8 4.41 (d, J=5.8 Hz, 2 H) 7.03 (t, J=5.7 Hz, 1 H) 7.14 (dd, J=7.7, 1.6 Hz, 1 H) 7.18-7.23 (m, 2 H) 7.25 (t, J=2.0 Hz, 1 H) 7.34-7.41 (m, 2 H) 7.73-7.80 (m, 2 H) 8.46 (s, 1 H) 8.48 (dd, J=4.8, 1.8 Hz, 1 H) 8.60 (d, J=2.3 Hz, 1 H) 9.45 (s, 1 H) HRMS (ESI+) calcd for C₂₂H₁₆BrClN₅ (MH+) 464.0274, found 464.0272.

### Example 71

### 4-(3-chlorophenylamino)-3-cyano-N,N-dimethyl-6-(pyridin-3-ylmethylamino)quinoline-8-carboxamide

Following the procedure described above in Example 4, 6-amino-4-(3-chlorophenylamino)-3-cyano-N,N-dimethylquinoline-8-carboxamide (21.5 mg, 0.0588 mmol) was reacted with 3-pyridinecarboxaldehyde (6.1 pL, 6.9 mg, 0.065 mmol) and NaCNBH₃ (4.1 mg, 0.065 mmol) in 1 mL EtOH. The crude product was purified by flash chromatography over silica gel (6% MeOH in CH₂Cl₂) and lyophilized to give a yellow solid (8.6 mg, 32% yield): ¹H NMR (400 MHz, DMSO-D₆) δ 2.67 (s, 3 H) 3.04 (s, 3 H) 4.42 (d, J=5.8 Hz, 2 H) 6.99 (t, J=5.8 Hz, 1 H) 7.13 (dd, J=8.0,1.6 Hz, 1 H) 7.17-7.21 (m, 1 H) 7.20 (s, 2 H) 7.25 (t, J=2.0 Hz, 1 H) 7.33-7.40 (m, 2 H) 7.77 (d, J=7.8 Hz, 1 H) 8.39 (s, 1 H) 8.47 (d, J=4.3 Hz, 1 H) 8.61 (s, 1 H) 9.42 (s, 1 H).

### Example 72

### 4-(3-chlorophenylamino)-6-(pyridin-3-ylmethylamino)quinoline-3-carbonitrile

Following the procedure described above in Example 4, 6-amino-4-(3-chlorophenylamino)quinoline-3-carbonitrile (0.100 g, 0.339 mmol) was reacted with 3-pyridinecarboxaldehyde (32 μL, 36 mg, 0.34 mmol) and NaCNBH₃ (23 mg, 0.37 mmol) in 40 mL EtOH. Purification of the crude product by flash chromatography over silica gel (5% MeOH in CH₂Cl₂), followed by lyophilization, gave a fluffy pale yellow solid (52 mg, 40% yield): ¹H NMR (400 MHz, DMSO-D₆) δ 4.39 (d, J=5.8 Hz, 2 H) 6.94 (t, J=5.9 Hz, 1 H) 7.05-7.21 (m, 4 H) 7.31-7.40 (m, 3 H) 7.71-7.78 (m, 2 H) 8.39 (s, 1 H) 8.46 (dd, J=4.7, 1.6 Hz, 1 H) 8.59 (d, J=1.5 Hz, 1 H) 9.33 (s, 1 H); HRMS (ESI+) calcd for C₂₂H₁₇ClN₅O (MH+) 386.1167, found 386.1169.

### Example 73

### N-(4-(3-chlorophenylamino)-3-cyano-6-(pyridin-3-ylmethylamino)quinolin-8-yl)benzamide

A microwave vial was charged with 8-bromo-4-(3-chlorophenylamino)-6-(pyridin-3-ylmethylamino)quinoline-3-carbonitrile (0.100 g, 0.215 mmol, prepared as described in Example 70 above), benzamide (63 mg, 0.52 mmol), CuI (20 mg, 0.105 mmol) and K₃PO₄ (91 mg, 0.43 mmol), and crimp-sealed. The vial was evacuated and backfilled with an inert gas, and a solution of trans-1,2-diaminocyclohexane (20 μL) in 4 mL dioxane was added. The vial was then heated in a microwave reactor at 150° C. for 30 minutes, until LC-MS analysis showed complete disappearance of the bromide starting material. The vial contents were then partitioned between EtOAc and brine, the aqueous layer extracted twice with additional EtOAc, and the combined organic layers washed with brine, dried over anhydrous MgSO₄, filtered, and evaporated. The crude product was purified first by flash chromatography over silica gel (3% MeOH in CH₂Cl₂), then by preparative HPLC, and lyophilized to give a fluffy, pale yellow solid (13 mg, 12% yield): ¹H NMR (400 MHz, DMSO-D₆) δ 4.43 (d, J=5.8 Hz, 2 H) 6.93 (d, J=2.5 Hz, 1 H) 7.10-7.27 (m, 4 H) 7.32-7.42 (m, 2 H) 7.59-7.71 (m, 3 H) 7.77 (d, J=8.1 Hz, 1 H) 7.99 (d, J=6.6 Hz, 2 H) 8.36-8.55 (m, 3 H) 8.61 (d, J=2.0 Hz, 1 H) 9.43 (s, 1 H) 10.50 (s, 1 H); HRMS (ESI+) calcd for C₂₉H₂₂ClN₆O (MH+) 505.1538, found 505.1537.

### Example 74

### 4-(3-chlorophenylamino)-6-(pyridin-3-ylmethylamino)quinoline-3,8-dicarbonitrile

In a microwave vial, under an inert atmosphere, 8-bromo-4-(3-chlorophenylamino)-6-(pyridin-3-ylmethylamino)quinoline-3-carbonitrile (0.100 g, 0.215 mmol), zinc cyanide (0.101 g, 0.860 mmol) and Pd(PPh₃)₄ (75 mg, 0.065 mmol) were taken up in 2 mL anhydrous DMF. The sealed vial was heated in a microwave reactor at 150° C. for 15 minutes, until LC-MS analysis showed complete consumption of the bromoquinoline. The vial contents were partitioned between EtOAc and brine and the aqueous layer extracted twice more with EtOAc. The combined organic layers were then washed with brine, dried over anhydrous MgSO₄, filtered and evaporated. The crude product was purified by preparative HPLC and lyophilized to give a fluffy yellow solid (6.9 mg, 7.8% yield): ¹H NMR (400 MHz, DMSO-D₆) δ 4.42 (d, J=6.1 Hz, 2 H) 7.03 (br s,1 H) 7.09 (br s,1 H) 7.31 (br s, 1 H) 7.37 (dd, J=7.5, 4.9 Hz, 1 H) 7.52-7.67 (m, 6 H) 7.78 (d, J=8.3 Hz, 1 H) 8.47 (d, J=5.3 Hz, 1 H) 8.61 (s, 1 H); HRMS (ESI+) calcd for C₂₃H₁₆ClN₆ (MH+) 411.1120, found 411.1131.

### Example 75

### N-(4-(3-chlorophenylamino)-3-cyano-6-(pyridin-3-ylmethylamino)quinolin-8-yl)formamide

Following the procedure described above in Example 73, 8-bromo-4-(3-chlorophenylamino)-6-(pyridin-3-ylmethylamino)quinoline-3-carbonitrile (0.100 g, 0.215 mmol, prepared as described in Example 71 above) was reacted with formamide (0.020 mL, 23 mg, 0.52 mmol), and the crude product was purified by preparative HPLC to give a glassy, brown solid (2.4 mg, 2.6% yield): ¹H NMR (400 MHz, DMSO-D₆) δ 4.38 (d, J=5.8 Hz, 2 H) 6.86 (d, J=1.8 Hz, 1 H) 7.05-7.24 (m, 4 H) 7.30-7.44 (m, 2 H) 7.74 (d, J=7.8 Hz, 1 H) 8.37-8.46 (m, 3 H) 8.55 (d, J=17.7 Hz, 2 H) 9.36 (s, 1 H) 10.56 (s, 1 H); HRMS (ESI+) calcd for C₂₃H₁₈ClN₆O (MH+) 429.1225, found 429.1223.

### Example 76

### 6-((1H-imidazol-5-yl)methylamino)-8-bromo-4-(3-chloro-4-fluorophenylamino)quinoline-3-carbonitrile

Step 1: In a microwave vial, 8-bromo-4-chloro-6-nitroquinoline-3-carbonitrile (4.00 g, 12.8 mmol) and 3-chloro-4-fluoroaniline (2.05 g, 14.1 mmol) were taken up in 20 mL EtOH. The vial was crimp-sealed and heated in a microwave reactor at 140° C. for 10 minutes. The cap was then removed, tin chloride dihydrate (16 g, 71 mmol) was added, and the vial was re-sealed and heated at 110° C. for 5 minutes. To work up the reaction, the contents of the vial were rinsed with EtOH into 300 mL ice water and neutralized with saturated NaHCO₃. The orange suspension was extracted with 4 aliquots of EtOAc, each equal to the volume of the aqueous layer. The combined organic layers were dried over anhydrous MgSO₄, filtered and evaporated, and the crude product was purified by flash chromatography over silica gel (3-5% MeOH in CH₂Cl₂) to give a brown solid 6-amino-8-bromo-4-(3-chloro-4-fluorophenylamino) quinoline-3-carbonitrile (2.50 g, 50% yield): ¹H NMR (400 MHz, DMSO-D₆) δ 6.05 (s, 2 H) 7.29-7.34 (m, 2 H) 7.49-7.55 (m, 2 H) 7.77 (d, J=2.3 Hz, 1 H) 8.53 (s, 1 H) 9.60 (s, 1 H); HRMS (ESI+) calcd for C₁₆H₁₀BrClFN₄ (MH+) 390.9756, found 390.9754.

Step 2: In a 1 L round-bottomed flask, the product from the previous step (5.40 g, 13.8 mmol) and 4(5)-imidazolecarboxaldehyde (1.33 g, 13.8 mmol) were taken up in 160 mL THF and 55 mL MeOH and stirred overnight. The solution was then acidified to pH 4 with acetic acid, NaCNBH₃ (0.58 g, 9.3 mmol) was added, and the mixture was allowed to stir overnight again. Solvent was then removed under reduced pressure, and the crude product purified by flash chromatography over silica gel (6-9% MeOH in CH₂Cl₂) and trituration with 165 mL boiling EtOH, to give a bright yellow powder (2.93 g, 45% yield): ¹H NMR (400 MHz, DMSO-D₆) δ 4.26 (d, J=5.1 Hz, 2 H) 6.67 (t, J=5.2 Hz, 1 H) 7.05 (s, 1 H) 7.25 (d, J=2.3 Hz, 1 H) 7.26-7.32 (m, 1 H) 7.45 (t, J=9.0 Hz, 1 H) 7.53 (dd, J=6.6, 2.5 Hz, 1 H) 7.62 (s,1 H) 7.79 (d, J=2.0 Hz, 1 H) 8.38 (s, 1 H) 9.47 (s, 1 H) 11.96 (br s,1 H); HRMS (ESI+) calcd for C₂₀H₁₄BrClFN₆ (MH+) 471.0131, found 471.0140.

### Example 77

### 8-bromo-4-(3-chloro-4-fluorophenylamino)-6-(pyridin-3-ylmethylamino) quinoline-3-carbonitrile

In a 250 mL round-bottomed flask, 6-amino-8-bromo-4-(3-chloro-4-fluorophenylamino)quinoline-3-carbonitrile (1.00 g, 2.55 mmol) was dissolved in 10 mL DMF. After the addition of 3-pyridinecarboxaldehyde (0.24 mL, 0.27 g, 2.6 mmol), the solution was allowed to stir for 5 hours. It was then acidified with acetic acid to pH 4, NaCNBH₃ (0.108 g, 1.71 mmol) was added, and the mixture was stirred overnight. To work up the reaction, 5 mL water was added, then the solution was poured into 50 mL 5% Na₂CO₃ and stirred vigorously for 30 minutes. The precipitate was collected by suction filtration, washing three times with water, and dried under vacuum overnight. The solid was extracted with 11 mL boiling EtOH, and the evaporated extract purified by flash chromatography over silica gel (5-65% EtOAc in CH₂Cl₂) and lyophilized to give a yellowish-brown powder (0.147 g, 12% yield): ¹H NMR (400 MHz, DMSO-D₆) δ 4.44 (d, J=5.6 Hz, 2 H) 7.00 (t, J=5.8 Hz, 1 H) 7.21-7.30 (m, 2 H) 7.37 (dd, J=7.7, 4.7 Hz, 1 H) 7.43 (t, J=9.0 Hz, 1 H) 7.50 (dd, J=6.4, 2.7 Hz, 1 H) 7.75 (d, J=1.8 Hz, 1 H) 7.79 (d, J=7.8 Hz, 1 H) 8.40 (s, 1 H) 8.48 (d, J=4.8 Hz, 1 H) 8.62 (d, J=2.0 Hz, 1 H) 9.44 (s, 1 H); HRMS (ESI+) calcd for C₂₂H₁₅BrClFN₅ (MH+) 482.0178, found 482.0179.

### Example 78

### 8-bromo-4-(3-chloro-4-fluorophenylamino)-6-(pyridin-2-ylmethylamino)quinoline-3-carbonitrile

Following the procedure described above in Example 4, 6-amino-8-bromo-4-(3-chloro-4-fluorophenylamino)quinoline-3-carbonitrile (0.500 g, 1.28 mmol) was reacted with 2-pyridinecarboxaldehyde (0.12 mL, 0.14 g, 1.3 mmol) in 15 mL THF and 5 mL MeOH; first for 3.5 hours to form the imine, then, after acidification with NaCNBH₃ (54 mg, 0.86 mmol) overnight. Solvent was removed under reduced pressure, and the crude product purified by preparative HPLC and lyophilized to give a fluffy yellow solid (0.13 g, 21 % yield): ¹H NMR (400 MHz, DMSO-D₆) δ 4.53 (d, J=5.6 Hz, 2 H) 7.07 (t, J=6.2 Hz, 1 H) 7.20-7.32 (m, 3 H) 7.33-7.44 (m, 2 H) 7.47 (dd, J=6.6, 2.8 Hz, 1 H) 7.76 (t, J=7.7 Hz, 1 H) 7.83 (s, 1 H) 8.39 (s, 1 H) 8.54 (d, J=4.8 Hz, 1 H) 9.38 (br s,1 H); HRMS (ESI+) calcd for C₂₂H₁₅BrClFN₅ (MH+) 482.0178, found 482.0188.

### Example 79

### 4-(3-chlorophenylamino)-8-iodo-6-(pyridin-3-ylmethylamino)quinoline-3-carbonitrile

A modification of the procedure described by J. Zanon, A. Klapars and S. Buchwald (J. Am. Chem. Soc. 2002, 124,14844-14845) was followed. A microwave vial was charged with 8-bromo-4-(3-chlorophenylamino)-6-(pyridin-3-ylmethylamino)quinoline-3-carbonitrile (0.100 g, 0.215 mmol, prepared as described in Example 70 above), CuI (20 mg, 0.105 mmol) and Nal (64 mg, 0.43 mmol). The vial was crimp-sealed, evacuated, and back-filled with an inert gas. A solution of N,N′-dimethylethylenediamine (0.020 mL, 17 mg, 0.19 mmol) in 4 mL dioxane was added via syringe, and the vial heated in a microwave reactor at 150° C. for 30 minutes, until LC-MS analysis showed complete consumption of the bromide starting material. The reaction mixture was then worked up as described above in Example 73, and purified by flash chromatography over silica gel (3% MeOH in CH₂Cl₂), to give a yellow powder (32 mg, 29% yield): ¹H NMR (400 MHz, DMSO-D₆) δ 4.40 (d, J=5.8 Hz, 2 H) 6.97 (t, J=5.8 Hz, 1 H) 7.11 (ddd, J=8.1, 2.0, 0.8 Hz, 1 H) 7.16-7.24 (m, 3 H) 7.33-7.39 (m, 2 H) 7.75 (dt, J=8.0, 1.9 Hz, 1 H) 8.02 (d, J=2.3 Hz, 1 H) 8.43 (s, 1 H) 8.47 (dd, J=4.8,1.5 Hz, 1 H) 8.59 (d, J=1.8 Hz, 1 H) 9.41 (s, 1 H); HRMS (ESI+) calcd for C₂₂H₁₆ClIN₅ (MH+) 512.0134, found 512.0142.

### Example 80

### N-benzyl-4-(3-chlorophenylamino)-3-cyano-6-(pyridin-3-ylmethylamino)quinoline-8-carboxamide

A modification of the procedure described by A Schoenberg and R. Heck (J. Org. Chem. 1974, 39(23), 3327-3331) was followed. A 100 mL 2-necked round-bottomed flask fitted with a condenser was charged with 8-bromo-4-(3-chlorophenylamino)-6-(pyridin-3-ylmethylamino)quinoline-3-carbonitrile (0.300 g, 0.646 mmol, prepared as described in Example 70 above), Pd(PPh₃)₂Cl₂ (68 mg, 0.097 mmol) and tri-n-butylamine (0.17 mL, 0.13 g, 0.71 mmol). The reaction apparatus was purged with CO gas, and kept under an atmosphere of CO over the course of the reaction by means of a balloon. Benzylamine (15 mL) was then added, and the mixture heated at 140° C. for 1.5 hours, until LC-MS analysis showed complete disappearance of the bromide starting material. The reaction was then cooled to RT and partitioned between EtOAc and brine. The aqueous layer was extracted twice more with EtOAc, and the combined organic layers washed successively with brine, 2M HOAc, brine, 5% Na₂CO₃ (2×), and brine. The EtOAc solution was then dried over anhydrous MgSO₄, filtered and evaporated, and purified by trituration with methanol to give a yellow solid (50 mg, 12% yield): ¹H NMR (400 MHz, DMSO-D₆) δ 4.45 (d, J=5.8 Hz, 2 H) 4.65 (d, J=6.1 Hz, 2 H) 7.14-7.41 (m, 12 H) 7.77 (d, J=8.1 Hz, 1 H) 8.21 (d, J=2.3 Hz, 1 H) 8.46 (dd, J=4.8, 1.3 Hz, 1 H) 8.48 (s, 1 H) 8.60 (d, J=2.0 Hz, 1 H) 9.54 (s, 1 H) 11.10 (t, J=6.2 Hz, 1 H); HRMS (ESI+) calcd for C₃₀H₂₃ClN₆O (MH+) 519.1695, found 519.1717.

### Example 81

### N-(4-(3-chlorophenylamino)-3-cyano-6-(pyridin-3-ylmethylamino)quinolin-8-yl)isobutyramide

Following the procedure described above in Example 73, 8-bromo-4-(3-chlorophenylamino)-6-(pyridin-3-ylmethylamino)quinoline-3-carbonitrile (0.280 g, 0.602 mmol, prepared as described in Example 70 above) was reacted with isobutyramide (0.126 g, 1.44 mmol) in the presence of Cul (56 mg, 0.29 mmol), K₃PO4 (0.256 g, 1.20 mmol) and N,N′-dimethylethylenediamine (0.056 mL, 46 mg, 0.526 mmol), in 7 mL dioxane. The crude product was purified by preparative HPLC and lyophilized to give a fluffy yellow solid (16 mg, 5.6% yield): ¹H NMR (400 MHz, DMSO-D₆) δ 1.16 (s, 6 H) 2.88 (s, 1 H) 4.39 (s, 2 H) 6.83 (s, 1 H) 7.09 (d, J=19.2 Hz, 2 H) 7.21 (s, 2 H) 7.34 (s, 2 H) 7.73 (s, 1 H) 8.39 (s, 2 H) 8.44 (s,1 H) 8.57 (s, 1 H) 9.34 (s, 1 H) 9.85 (s, 1 H); HRMS (ESI+) calcd for C₂₆H₂₄ClN₆O (MH+) 471.1695, found 471.1693.

### Example 82

### 4-(3-chloro-4-fluorophenylamino)-3-cyano-6-(cyclohexylmethylamino)-N,N-dimethylquinoline-8-carboxamide

Step 1: In a 250 mL round-bottomed flask fitted with a condenser, 4-chloro-3-cyano-N,N-dimethyl-6-nitroquinoline-8-carboxamide (4.97 g, 16.3 mmol) and 3-chloro-4-fluoroaniline (2.61 g, 17.9 mmol) were taken up in 60 mL EtOH and refluxed for 30 minutes. The mixture was allowed to cool for 30 minutes, and tin chloride dihydrate (1 8.4 g, 81.5 mmol) was added. The mixture was then refluxed for an additional 30 minutes, cooled to RT, and poured into 300 mL ice water. After neutralization with saturated NaHCO₃, the orange suspension was extracted 5 times with 700 mL portions of CHCl₃. The organic extracts were dried over anhydrous MgSO₄, filtered, combined and evaporated, and the crude product purified by flash chromatography over silica gel (5-9% MeOH in CH₂Cl₂), to give a brown powder 6-amino-4-(3-chloro-4-fluorophenylamino)-3-cyano-N,N-dimethylquinoline-8 carboxamide (5.09 g, 81% yield): ¹H NMR (400 MHz, DMSO-D₆) δ 2.68 (s, 3 H) 3.04 (s, 3 H) 5.86 (s, 2 H) 7.09 (d, J=2.0 Hz, 1 H) 7.16-7.23 (m, 2 H) 7.35-7.44 (m, 2 H) 8.34 (s, 1 H) 9.44 (s, 1 H); HRMS (ESI+) calcd for C₁₉H₁₆ClFN₅O (MH+) 384.1022, found 384.1029.

Step 2: In an 18x150mm test tube, 6-amino-4-(3-chloro-4-fluorophenylamino)-3-cyano-N,N-dimethylquinoline-8-carboxamide (0.300 g, 0.782 mmol) and cyclohexanecarboxaldehyde (0.094 mL, 88 mg, 0.782 mmol) were taken up in 9 mL THF and 3 mL MeOH and stirred overnight. The mixture was then acidified to pH 4 with acetic acid, NaCNBH₃ (33 mg, 0.52 mmol) was added, and it was allowed to stir overnight again. The yellow precipitate was then collected by suction filtration, washed with MeOH, and dried under vacuum, giving pure product as a bright yellow powder (71 mg, 20% yield): ¹H NMR (400 MHz, DMSO-D₆) δ 0.90-1.04 (m, 2 H) 1.12-1.29 (m, 3 H) 1.50-1.86 (m, 6 H) 2.67 (s, 3 H) 2.93-2.99 (m, 2 H) 3.04 (s, 3 H) 6.38 (t, J=5.4 Hz, 1 H) 6.98 (d, J=2.3 Hz, 1 H) 7.15 (d, J=2.5 Hz, 1 H) 7.25 (ddd, J=8.8, 4.1, 2.8 Hz, 1 H) 7.43 (t, J=9.0 Hz, 1 H) 7.48 (dd, J=6.6, 2.8 Hz, 1 H) 8.31 (s, 1 H) 9.38 (s, 1 H); HRMS (ESI+) calcd for C₂₆H₂₈ClFN₅O (MH+) 480.1961, found 480.1960.

### Example 83

### 4-(3-chloro-4-fluorophenylamino)-3-cyano-N,N-dimethyl-6-((1-methyl-1H-benzo[d]imidazol-2-yl)methylamino)quinoline-8-carboxamide

The procedure described above in Example 82 was followed. Because the product did not precipitate out of the reaction mixture, solvent was removed under reduced pressure, and the crude product taken up in 6 mL MeOH, stirred vigorously, and filtered. The precipitate was washed with MeOH and dried under vacuum to give a pale yellow powder (83 mg, 20% yield): ¹H NMR (400 MHz, DMSO-D₆) δ 2.67 (s, 3 H) 3.04 (s, 3 H) 3.83 (s, 3 H) 4.69 (dd, J=4.9, 2.4 Hz, 2 H) 6.95 (t, J=5.3 Hz, 1 H) 7.16-7.21 (m, 1 H) 7.22-7.34 (m, 3 H) 7.39 (d, J=2.5 Hz, 1 H) 7.44 (t, J=9.0 Hz, 1 H) 7.51-7.62 (m, 3 H) 8.36 (s, 1 H) 9.45 (s, 1 H); HRMS (ESI+) calcd for C₂₈H₂₄ClFN₇O (MH+) 528.1710, found 528.1716.

### Example 84

### 4-(3-chloro-4-fluorophenylamino)-3-cyano-6-(furan-2-ylmethylamino)-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 2-furaldehyde (0.065 mL, 75 mg, 0.78 mmol) and NaCNBH₃ (33 mg, 0.52 mmol). The crude product was purified by trituration with 6 mL MeOH, to give a bright yellow powder (0.136 g, 38% yield): ¹H NMR (400 MHz, DMSO-D₆) δ 2.67 (s, 3 H) 3.04 (s, 3 H) 4.41 (d, J=5.8 Hz, 2 H) 6.34-6.43 (m, 2 H) 6.85 (t, J=6.2 Hz, 1 H) 7.19 (d, J=2.3 Hz, 1 H) 7.25-7.33 (m, 2 H) 7.44 (t, J=9.1 Hz, 1 H) 7.53 (dd, J=6.6, 2.3 Hz, 1 H) 7.58-7.63 (m, 1 H) 8.33 (s, 1 H) 9.42 (s, 1 H); HRMS (ESI+) calcd for C₂₄H₂₀ClFN₅O₂ (MH+) 464.1284, found 464.1288.

### Example 85

### 4-(3-chloro-4-fluorophenylamino)-3-cyano-6-(3-cyanobenzylamino)-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 3-cyanobenzaldehyde (103 mg, 0.782 mmol) and NaCNBH₃ (33 mg, 0.52 mmol). The crude product was purified by preparative HPLC and lyophilized to give a fluffy, bright yellow solid (0.131 g, 34% yield): ¹H NMR (400 MHz, DMSO-D₆) δ 2.67 (s, 3 H) 3.04 (s, 3 H) 4.48 (d, J=6.1 Hz, 2 H) 7.01 (t, J=5.8 Hz, 1 H) 7.19 (s, 2 H) 7.24 (ddd, J=8.8, 4.2, 2.7 Hz, 1 H) 7.41 (t, J=9.0 Hz, 1 H) 7.47 (dd, J=6.4, 2.7 Hz, 1 H) 7.56 (t, J=7.7 Hz, 1 H) 7.73 (t, J=7.5 Hz, 2 H) 7.84 (s, 1 H) 8.34 (s, 1 H) 9.41 (br s,1 H); HRMS (ESI+) calcd for C₂₇H₂₁ClFN₆O (MH+) 499.1444, found 499.1443.

### Example 86

### 4-(3-chloro-4-fluorophenylamino)-3-cyano-6-(4-cyanobenzylamino)-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 4-cyanobenzaldehyde (103 mg, 0.782 mmol) and NaCNBH₃ (33 mg, 0.52 mmol). The crude product was purified by preparative HPLC and lyophilized to give a fluffy, bright yellow solid (0.107 g, 28% yield): ¹H NMR (400 MHz, DMSO-D₆) δ 2.67 (s, 3 H) 3.04 (s, 3 H) 4.52 (d, J=6.3 Hz, 2 H) 7.07 (t, J=6.2 Hz, 1 H) 7.14-7.24 (m, 3 H) 7.40 (t, J=9.0 Hz, 1 H) 7.45 (dd, J=6.6, 2.5 Hz, 1 H) 7.55 (d, J=8.3 Hz, 2 H) 7.80 (d, J=8.6 Hz, 2 H) 8.34 (s, 1 H) 9.39 (br s, 1 H); HRMS (ESI+) calcd for C₂₇H₂₁ClFN₆O (MH+) 499.1444, found 499.1442.

### Example 87

### 6-((1H-imidazol-5-yl)methylamino)-4-(3-chloro-4-fluorophenylamino)-3-cyano-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 4(5)-imidazolecarboxaldehyde (75 mg, 0.78 mmol) and NaCNBH₃ (33 mg, 0.52 mmol). The crude product was purified by preparative HPLC and lyophilized to give a fluffy, bright yellow solid (96 mg, 26% yield): ¹H NMR (400 MHz, DMSO-D₆) δ 2.67 (s, 3 H) 3.03 (s, 3 H) 4.28 (d, J=5.1 Hz, 2 H) 6.62 (t, J=5.3 Hz, 1 H) 7.05 (s, 1 H) 7.19-7.33 (m, 3 H) 7.44 (t, J=9.1 Hz, 1 H) 7.53 (dd, J=6.6, 2.8 Hz, 1 H) 7.62 (s, 1 H) 8.31 (s, 1 H) 9.47 (br s,1 H); HRMS (ESI+) calcd for C₂₃H₂₀ClFN₇O (MH+) 464.1397, found 464.1401.

### Example 88

### 4-(3-chloro-4-fluorophenylamino)-3-cyano-6-((5-(hydroxymethyl)furan-2-yl)methylamino)-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 5-(hydroxymethyl)furfural (99 mg, 0.78 mmol) and NaCNBH₃ (33 mg, 0.52 mmol). The crude product was purified by preparative HPLC and lyophilized to give a fluffy, bright yellow solid (47 mg, 12% yield): ¹H NMR (400 MHz, DMSO-D₆) δ 2.67 (s, 3 H) 3.04 (s, 3 H) 4.34 (s, 2 H) 4.39 (d, J=5.6 Hz, 2 H) 5.17 (br s,1 H) 6.19 (d, J=3.0 Hz, 1 H) 6.29 (d, J=3.0 Hz, 1 H) 6.86 (t, J=5.8 Hz, 1 H) 7.19 (d, J=2.5 Hz, 1 H) 7.24-7.34 (m, 2 H) 7.44 (t, J=9.0 Hz, 1 H) 7.53 (dd, J=6.7, 2.7 Hz, 1 H) 8.32 (s, 1 H) 9.46 (br s,1 H); HRMS (ESI+) calcd for C₂₅H₂₂ClFN₅O₃ (MH+) 494.1390, found 494.1392.

### Example 89

### 6-((1H-pyrazol-5-yl)methylamino)-4-(3-chloro-4-fluorophenylamino)-3-cyano-N,N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with pyrazol-3-carbaldehyde (75 mg, 0.78 mmol) and NaCNBH₃ (33 mg, 0.52 mmol). The crude product was purified by preparative HPLC and lyophilized to give a yellow powder (41 mg, 11 % yield): ¹H NMR (400 MHz, DMSO-D₆) δ 2.67 (s, 3 H) 3.04 (s, 3 H) 4.37 (d, J=5.1 Hz, 2 H) 6.26 (d, J=1.8 Hz, 1 H) 6.71 (br s,1 H) 7.22 (d, J=2.0 Hz, 1 H) 7.25-7.34 (m, 2 H) 7.44 (t, J=9.0 Hz, 1 H) 7.52 (dd, J=6.6, 2.5 Hz, 1 H) 7.63 (br s,1 H) 8.32 (s, 1 H) 9.47 (br s,1 H) 12.68 (br s,1 H); HRMS (ESI+) calcd for C₂₃H₂₀ClFN₇O (MH+) 464.1397, found 464.1402.

### Example 90

### 4-(3-chloro-4-fluorophenylamino)-3-cyano-6-((1,3-dimethyl-1-H-pyrazol-5-yl)methylamino)-N, N-dimethylquinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 1,3-dimethyl-1H-pyrazole-5-carbaldehyde (97 mg, 0.78 mmol) and NaCNBH₃ (33 mg, 0.52 mmol). The crude product was purified by preparative HPLC and lyophilized to give a fluffy yellow solid (69 mg, 18% yield): ¹H NMR (400 MHz, DMSO-D₆) 6 2.07 (s, 3 H) 2.67 (s, 3 H) 3.04 (s, 3 H) 3.72 (s, 3 H) 4.36 (d, J=5.3 Hz, 2 H) 6.03 (s, 1 H) 6.78 (t, J=5.1 Hz, 1 H) 7.19 (d, J=2.5 Hz, 1 H) 7.25-7.34 (m, 2 H) 7.44 (t, J=9.0 Hz, 1 H) 7.52 (dd, J=6.6, 2.8 Hz, 1 H) 8.35 (s, 1 H) 9.49 (br s, 1 H); HRMS (ESI+) calcd for C₂₅H₂₃ClFN₇O (MH+) 492.1710, found 492.1709.

### Example 91

### 4-(3-chloro-4-fluorophenylamino)-3-cyano-N,N-dimethyl-6-(pyridin-2-ylmethylamino)quinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with 2-pyridinecarboxaldehyde (74 μL, 84 mg, 0.78 mmol) and NaCNBH₃ (33 mg, 0.52 mmol). The crude product was purified twice by preparative HPLC to give a yellowish-brown solid (4.5 mg, 1.2% yield): ¹H NMR (400 MHz, DMSO-D₆) δ 2.66 (s, 3 H) 3.03 (s, 3 H) 4.53 (d, J=5.8 Hz, 2 H) 7.01 (t, J=6.1 Hz, 1 H) 7.18-7.30 (m, 4 H) 7.35-7.42 (m, 2 H) 7.46 (dd, J=6.6, 2.5 Hz, 1 H) 7.74 (dt, J=7.7, 1.8 Hz, 1 H) 8.31 (s, 1 H) 8.53 (d, J=4.0 Hz, 1 H) 9.45 (br s, 1 H); HRMS (ESI+) calcd for C₂₅H₂₁ClFN₆O (MH+) 475.1444, found 475.1436.

### Example 92

### 4-(3-chloro-4-fluorophenylamino)-3-cyano-N,N-dimethyl-6-(3-methylbenzylamino)quinoline-8-carboxamide

Following the procedure described above in Example 4, 6-amino-4-(3-chloro-4-fluorophenylamino)-3-cyano-N,N-dimethylquinoline-8-carboxamide (0.300 g, 0.782 mmol) was reacted with m-tolualdehyde (92 μL, 94 mg, 0.78 mmol) and NaCNBH₃ (33 mg, 0.52 mmol). The crude product was purified by trituration with 7 mL boiling MeOH, to give a bright yellow powder (0.151 g, 40% yield): ¹H NMR (400 MHz, DMSO-D₆) δ 2.27 (s, 3 H) 2.66 (s, 3 H) 3.02 (s, 3 H) 4.34 (d, J=5.6 Hz, 2 H) 6.88 (t, J=5.7 Hz, 1 H) 7.06 (d, J=7.1 Hz, 1 H) 7.13-7.25 (m, 6 H) 7.41 (t, J=9.0 Hz, 1 H) 7.47 (dd, J=6.6,2.8 Hz, 1 H) 8.32 (s, 1 H) 9.39 (s, 1 H); HRMS (ESI+) calcd for C₂₇H₂₄ClFN₅O (MH+) 488.1648, found 488.1643.

### Example 93

### 4-(3-chloro-4-fluorophenylamino)-3-cyano-N-isopropyl-6-(pyridin-3-ylmethylamino)quinoline-8-carboxamide

The procedure used was a modification of those described by A. Schoenberg and R. Heck (J. Org. Chem. 1974, 39(23), 3327-3331), and M. Larhed et al. (J. Comb. Chem. 2002, 4,109-111). A microwave vial was charged with 8-bromo-4-(3-chloro-4-fluorophenylamino)-6-(pyridin-3-ylmethylamino)quinoline-3-carbonitrile as prepared in Example 77 (0.100 g, 0.207 mmol), Mo(CO)₆ (27 mg, 0.10 mmol) and Pd(PPh₃)₂Cl₂ (29 mg, 0.041 mmol), and crimp-sealed. The vial was then purged with CO gas, and isopropylamine (5 mL), tri-n-butylamine (0.055 mL, 43 mg, 0.23 mmol) and toluene (1 mL) were added. The vial was then heated in a microwave reactor at 150° C. for 15 minutes, until LC-MS analysis showed complete disappearance of 8-bromo-4-(3-chloro-4-fluorophenylamino)-6-(pyridin-3-ylmethylamino)quinoline-3-carbonitrile. This was repeated on the same scale with 3 additional vials, and the contents of the 4 vials then worked up together by partitioning between EtOAc and brine, extracting the aqueous layer twice more with EtOAc, washing the combined organic layers with brine, drying over anhydrous MgSO₄, filtering, and evaporating. The crude product was purified by preparative HPLC and lyophilized to give a yellow solid (50 mg, 12% yield): ¹H NMR (400 MHz, DMSO-D₆) δ 1.22 (d, J=6.6 Hz, 6 H) 4.45 (d, J=5.6 Hz, 2 H) 7.10-7.51 (m, 6 H) 7.77 (d, J=7.6 Hz, 1 H) 8.15 (d, J=2.0 Hz, 1 H) 8.39 (s, 1 H) 8.45 (d, J=3.3 Hz, 1 H) 8.60 (s,1 H) 9.61 (br s,1 H) 10.68 (d, J=6.6 Hz, 1 H); HRMS (ESI+) calcd for C₂₆H₂₃ClFN₆O (MH+) 489.1601, found 489.1595.

### Example 94

### 4-(3-chloro-4-fluorophenylamino)-3-cyano-N,N-dimethyl-6-(pyridin-3-ylmethylamino)quinoline-8-carboxamide

Following the procedure described above in Example 4, 6-amino-4-(3-chloro-4-fluorophenylamino)-3-cyano-N,N-dimethylquinoline-8-carboxamide (0.27 g, 0.70 mmol) was reacted with 3-pyridinecarboxaldehyde (0.066 mL, 75 mg, 0.70 mmol) and NaCNBH₃ (30 mg, 0.47 mmol) in 9 mL THF and 3 mL MeOH. The crude product was purified first by trituration with 5 mL MeOH, then by preparative HPLC, and lyophilized to give a yellow solid (24 mg, 7.2% yield): ¹H NMR (400 MHz, DMSO-D₆) δ 2.65 (s, 3 H) 3.02 (s, 3 H) 4.43 (d, J=5.6 Hz, 2 H) 6.94 (t, J=5.7 Hz, 1 H) 7.18 (s, 1 H) 7.22-7.28 (m, 2 H) 7.33-7.44 (m, 2 H) 7.48 (dd, J=6.3, 2.5 Hz, 1 H) 7.78 (d, J=8.1 Hz, 1 H) 8.31 (s, 1 H) 8.46 (d, J=4.0 Hz, 1 H) 8.62 (s, 1 H) 9.45 (br s, 1 H); HRMS (ESI+) calcd for C₂₅H₂₁ClFN₆O (MH+) 475.1444, found 475.1437.

### Example 95

### 4-(3-chloro-4-fluorophenylamino)-6-(phenylamino)quinoline-3-carbonitrile

A modification of the procedure described by J. Wolfe and S. Buchwald (Org. Synth. 2002, 78, 23-25) was followed. A microwave vial was charged with 6-bromo-4-(3-chloro-4-fluorophenylamino)quinoline-3-carbonitrile (0.100 g, 0.266 mmol), Pd(OAc)₂ (20 mg, 0.089 mmol), (±)-BINAP (20 mg, 0.032 mmol) and Cs₂CO₃, and crimp-sealed. The vial was purged and backfilled with an inert gas, and a solution of aniline (0.30 mL, 0.31 g, 3.3 mmol) in 5 mL anhydrous THF was added. The vial was then heated in a microwave reactor at 180° C. for 2 hours, until LC-MS analysis showed complete consumption of starting material. The vial contents were partitioned between EtOAc and brine, the aqueous layer extracted twice more with EtOAc, and the combined organic layers washed with brine, dried over anhydrous MgSO₄, filtered, and evaporated. The crude product was purified by preparative HPLC and lyophilized to give a yellow solid (9.2 mg, 8.9% yield): ¹H NMR (400 MHz, DMSO-D₆) δ 6.90 (t, J=7.5 Hz, 1 H) 7.14 (d, J=8.1 Hz, 3 H) 7.24 (t, J=7.6 Hz, 2 H) 7.33-7.41 (m, 2 H) 7.54 (d, J=9.4 Hz, 1 H) 7.77 (s, 1 H) 7.83 (d, J=9.1 Hz, 1 H) 8.44 (s, 1 H) 8.69 (s, 1 H) 9.56 (s, 1 H); HRMS (ESI+) calcd for C₂₂H₁₅ClFN₄ (MH+) 389.0964, found 389.0959.

### Example 96

### 6-((1H-imidazol-5-yl)methylamino)-8-bromo-4-(cyclopentylamino)quinoline-3-carbonitrile

Step 1: Following the procedure described above in Example 68, 8-bromo-4-chloro-6-nitroquinoline-3-carbonitrile (1.00 g, 3.20 mmol) was reacted with cyclopentylamine (0.63 mL, 0.54 g, 6.4 mmol). The crude product was purified by flash chromatography over silica gel (5% EtOAc in CH₂Cl₂) to give a fluffy, bright yellow solid 8-bromo-4-(cyclopentylamino)-6-nitroquinoline-3-carbonitrile (0.405 g, 35% yield): ¹H NMR (400 MHz, DMSO-D₆) δ 1.54-1.69 (m, 2 H) 1.72-1.93 (m, 4 H) 2.03-2.15 (m, 2 H) 4.69-4.82 (m, 1 H) 8.50 (d, J=7.6 Hz, 1 H) 8.75 (d, J=1.5 Hz, 2 H) 9.54 (d, J=2.5 Hz, 1 H); HRMS (ESI+) calcd for C₁₅H₁₄BrN₄O₂ (MH+) 361.0295, found 361.0293.

Step 2: Following the procedure described above in Example 69, 8-bromo-4-(cyclopentylamino)-6-nitroquinoline-3-carbonitrile (0.354 g, 0.980 mmol) was reacted with tin chloride dihydrate (1.11 g, 4.90 mmol). Work up was also as described, except that the neutralized aqueous suspension was extracted with EtOAc (4×) instead of CHCl₃. Evaporation of the EtOAc gave pure product 6-amino-8-bromo-4-(cyclopentylamino)quinoline-3-carbonitrile as a brown powder (0.252 g, 78% yield): ¹H NMR (400 MHz, DMSO-D₆) 6 1.51-1.66 (m, 2 H) 1.69-1.84 (m, 4 H) 1.97-2.11 (m, 2 H) 4.56-4.69 (m, 1 H) 5.65 (s, 2 H) 7.21 (d, J=7.3 Hz, 1 H) 7.27 (d, J=2.3 Hz, 1 H) 7.52 (d, J=2.0 Hz, 1 H) 8.22 (s, 1 H); HRMS (ESI+) calcd for C₁₅H₁₆BrN₄ (MH+) 331.0553, found 331.0557.

Step 3: Following the procedure described above in Example 4, 6-amino-8-bromo-4-(cyclopentylamino)quinoline-3-carbonitrile (0.224 g, 0.676 mmol) was reacted with 4(5)-imidazolecarboxaldehyde (65 mg, 0.68 mmol) and NaCNBH₃ (29 mg, 0.45 mmol) in 4.5 mL THF and 1.5 mL MeOH. The crude product was taken up in MeOH, and a tan powder precipitated. This powder was collected, washed with MeOH, and dried under vacuum to give pure product (62 mg, 22% yield): ¹H NMR (400 MHz, DMSO-D₆) δ 1.61 (br. s, 2 H) 1.70-1.86 (m, 4 H) 2.01-2.14 (m, 2 H) 4.29 (d, J=5.3 Hz, 2 H) 4.59-4.74 (m, 1 H) 6.45 (t, J=5.2 Hz, 1 H) 7.05 (s, 1 H) 7.13-7.22 (m, 2 H) 7.61 (d, J=1.0 Hz, 1 H) 7.66 (d, J=2.3 Hz, 1 H) 8.24 (s, 1 H) 11.95 (br s, 1 H); HRMS (ESI+) calcd for C₁₉H₂₀BrN₆ (MH+) 411.0928, found 411.0939.

### Example 97

### 6-((1H-imidazol-5-yl)methylamino)-8-bromo-4-(cycloheptylamino)quinoline-3-carbonitrile

Step 1: Following the procedure described above in Example 68, 8-bromo-4-chloro-6-nitroquinoline-3-carbonitrile (1.00 g, 3.20 mmol) was reacted with cycloheptylamine (0.82 mL, 0.72 g, 6.4 mmol). The crude product was purified by flash chromatography over silica gel (gradient elution, 5-50% EtOAc in CH₂Cl₂) to give a fluffy, bright yellow solid 8-bromo-4-(cycloheptylamino)-6-nitroquinoline-3-carbonitrile (WAY-199056, 0.293 g, 23% yield): ¹H NMR (400 MHz, DMSO-D₆) δ 1.46-1.67 (m, 6 H) 1.68-1.88 (m, 4 H) 2.00-2.11 (m, 2 H) 4.47-4.64 (m, 1 H) 8.50 (d, J=8.6 Hz, 1 H) 8.73 (s, 1 H) 8.74 (d, J=2.3 Hz, 1 H) 9.53 (d, J=2.3 Hz, 1 H); HRMS (ESI+) calcd for C₁₇H₁₈BrN₄O₂ (MH+) 389.0608, found 389.0606.

Step 2: Following the procedure described above in Example 69, 8-bromo-4-(cycloheptylamino)-6-nitroquinoline-3-carbonitrile (0.234 g, 0.601 mmol) was reacted with tin chloride dihydrate (0.68 g, 3.01 mmol). Workup was also as described, except that the neutralized aqueous suspension was extracted with EtOAc (4×) instead of CHCl₃. Evaporation of the EtOAc gave pure product 6-amino-8-bromo-4-(cycloheptylamino)quinoline-3-carbonitrile as a purplish-brown powder (0.164 g, 76% yield): ¹H NMR (400 MHz, DMSO-D₆) δ 1.46-1.80 (m, 10 H) 1.94-2.05 (m, 2 H) 4.33-4.51 (m, 1 H) 5.64 (s, 2 H) 7.14 (d, J=8.8 Hz, 1 H) 7.27 (d, J=2.3 Hz, 1 H) 7.52 (d, J=2.3 Hz, 1 H) 8.21 (s, 1 H); HRMS (ESI+) calcd for C₁₇H₂₀BrN₄O (MH+) 359.0866, found 359.0873.

Step 3: Following the procedure described above in Example 4, 6-amino-8-bromo-4-(cycloheptylamino)quinoline-3-carbonitrile (0.137 g, 0.381 mmol) was reacted with 4(5)-imidazolecarboxaldehyde (37 mg, 0.38 mmol) and NaCNBH₃ (16 mg, 0.26 mmol) in 4.5 mL THF and 1.5 mL MeOH. The crude product was taken up in MeOH, and a beige powder precipitated. This powder was collected, washed with MeOH, and dried under vacuum to give pure product (37 mg, 22% yield): ¹H NMR (400 MHz, DMSO-D₆) δ 1.44-1.86 (m, 10 H) 1.96-2.13 (m, 2 H) 4.29 (d, J=4.8 Hz, 2 H) 4.38-4.54 (m, 1 H) 6.44 (t, J=4.2 Hz, 1 H) 7.05 (s, 1 H) 7.10-7.24 (m, 2 H) 7.63 (d, J=17.4 Hz, 2 H) 8.23 (s, 1 H) 11.97 (br s,1 H); HRMS (ESI+) calcd for C₂₁H₂₄BrN₆ (MH+) 439.1241, found 439.1253.

### Example 98

### 6-((1H-imidazol-5-yl)methylamino)-8-bromo-4-(tert-butylamino)quinoline-3-carbonitrile

Step 1: Following the procedure described above in Example 68, 8-bromo-4-chloro-6-nitroquinoline-3-carbonitrile (1.00 g, 3.20 mmol) was reacted with tert-butylamine (0.68 mL, 0.46 g, 13 mmol). The crude product was purified by flash chromatography over silica gel (gradient elution, 1-20% EtOAc in CH₂Cl₂) to give pure product 8-bromo-4-(tert-butylamino)-6-nitroquinoline-3-carbonitrile (0.518 g, 46% yield): ¹H NMR (400 MHz, DMSO-D₆) δ 1.62 (s, 9 H) 7.83 (s, 1 H) 8.79 (d, J=2.5 Hz, 1 H) 8.87 (s, 1 H) 9.26 (d, J=2.3 Hz, 1 H); HRMS (ESI+) calcd for C₁₄H₁₄BrN₄O₂ (MH+) 349.0295, found 349.0297.

Step 2: In a 25 mL round-bottomed flask fitted with a condenser, 8-bromo-4-tert-butylamino-6-nitroquinoline-3-carbonitrile (0.257 g, 0.736 mmol) was taken up in 4 mL MeOH and 2 mL water, and iron powder (0.370 g, 6.62 mmol) and NH₄Cl (0.591 g, 11.0 mmol) were added. The mixture was heated at reflux for 1 hour, until LC-MS analysis showed complete conversion of nitroquinoline to aniline. After cooling to RT, the reaction mixture was diluted with EtOAc, basified with saturated NaHCO₃, dried by addition of anhydrous MgSO₄, filtered, and evaporated to give the product 6-amino-8-bromo-4-(tert-butylamino)quinoline-3-carbonitrile as a golden-yellow powder (0.213 g, 90% yield): ¹H NMR (400 MHz, DMSO-D₆) δ 1.51 (s, 9 H) 5.83 (s, 2 H) 6.18 (s, 1 H) 7.13 (d, J=2.3 Hz, 1 H) 7.56 (d, J=2.3 Hz, 1 H) 8.35 (s, 1 H); HRMS (ESI+) calcd for C₁₄H₁₆BrN₄ (MH+) 319.0553, found 319.0557.

Step 3: Following the procedure described above in Example 4, 6-amino-8-bromo-4-tert-butylaminoquinoline-3-carbonitrile (137 mg, 0.429 mmol) was reacted with 4(5)-imidazolecarboxaldehyde (41 mg, 0.43 mmol) and NaCNBH₃ (18 mg, 0.29 mmol) in 4.5 mL THF and 1.5 mL MeOH. The crude product was purified by preparative HPLC and lyophilized to give a beige solid (68 mg, 40% yield): ¹H NMR (400 MHz, DMSO-D₆) δ 1.49 (s, 9 H) 4.28 (d, J=4.3 Hz, 2 H) 6.18 (s, 1 H) 6.68 (t, J=4.9 Hz, 1 H) 7.03 (s, 1 H) 7.10 (s, 1 H) 7.60 (s, 1 H) 7.69 (s, 1 H) 8.39 (s, 1 H); HRMS (ESI+) calcd for C₁₈H₂₀BrN₆ (MH+) 399.0928, found 399.0912.

### Example 99

### 4-(3-cyano-6-(pyridin-3-ylmethylamino)quinolin-4-ylamino)benzamide

Step 1 Following the procedure described above in Example 69, 6-nitro-4-oxo-1,4-dihydroquinoline-3-carbonitrile (5.00 g, 23.2 mmol) was reacted with oxalyl chloride (4.0 mL, 5.9 g, 46 mmol) in 50 mL DCE, with 0.42 mL DMF. Pure product 4-chloro-6-nitroquinoline-3-carbonitrile was obtained as a brown solid (5.00 g, 92% yield): ¹H NMR (400 MHz, DMSO-D₆) δ 8.41 (d, J=9.1 Hz, 1 H) 8.70 (dd, J=9.1, 2.5 Hz, 1 H) 9.04 (d, J=2.5 Hz, 1 H) 9.41 (s, 1 H).

Step 2: Following the procedure described above in Example 76, 4-chloro-6-nitroquinoline-3-carbonitrile (0.500 g, 2.14 mmol) was reacted first with 4-aminobenzamide (0.320 g, 2.35 mmol), then with tin chloride dihydrate (2.41 g, 10.7 mmol), in 5 mL EtOH. The crude product was purified by trituration with 17 mL boiling EtOH, to give a pumpkin-orange colored powder 4-(6-amino-3-cyanoquinolin-4-ylamino)benzamide (0.210 g, 32% yield): ¹H NMR (400 MHz, DMSO-D₆) δ 7.38-7.52 (m, 5 H) 7.87 (d, J=9.1 Hz, 1 H) 7.97 (d, J=8.6 Hz, 2 H) 8.05 (s, 1 H) 8.83 (s, 1 H) 10.90 (s, 1 H); HRMS (ESI+) calcd for C₁₇H₁₄N₅O (MH+) 304.1193, found 304.1195.

Step 3: Following the procedure described above in Example 4, 4-(6-amino-3-cyanoquinolin-4-ylamino)benzamide (0.145 g, 0.478 mmol) was reacted with 3-pyridinecarboxaldehyde (0.045 mL, 51 mg, 0.48 mmol) and NaCNBH₃ (20 mg, 0.32 mmol) in 5 mL THF and 14 mL MeOH. The crude product was purified by preparative HPLC and lyophilized to give a yellowish-brown solid (29 mg, 15% yield): ¹H NMR (400 MHz, DMSO-D₆) δ 4.32 (d, J=5.6 Hz, 2 H) 6.90 (t, J=5.7 Hz, 1 H) 7.04-7.09 (m, 3 H) 7.18 (s, 1 H) 7.25-7.35 (m, 2 H) 7.65-7.72 (m, 2 H) 7.78-7.86 (m, 3 H) 8.37 (s, 1 H) 8.39 (dd, J=4.7,1.39 Hz, 1 H) 8.52 (d, J=1.8 Hz, 1 H) 9.36 (s, 1 H); HRMS (ESI+) calcd for C₂₃H₁₉N₆ (MH+) 395.1615, found 395.1615.

### Example 100

### 4-(3-chlorophenylamino)-6-(pyridin-3-ylmethylamino)-8-(thiophen-3-yl)quinoline-3-carbonitrile

A microwave vial was charged with 8-bromo-4-(3-chlorophenylamino)-6-(pyridin-3-ylmethylamino)quinoline-3-carbonitrile as prepared in Example 70 above (80 mg, 0.17 mmol), 3-thiopheneboronic acid (26 mg, 0.21 mmol), Pd(PPh₃)₂Cl₂ (12 mg, 0.017 mmol), Na₂CO₃ (22 mg, 0.21 mmol), and 1.5 mL each DME, EtOH and water. The vial was crimp-sealed and heated in a microwave reactor at 140° C. for 30 minutes, until LC-MS analysis showed complete consumption of 8-bromo-4-(3-chlorophenylamino)-6-(pyridin-3-ylmethylamino)quinoline-3-carbonitrile. The contents of the vial were then partitioned between EtOAc and brine, the aqueous layer extracted twice more with EtOAc, and the combined organic layers washed with brine, dried over anhydrous MgSO₄, filtered and evaporated. Crude product was purified by preparative HPLC and lyophilized to give a light brown solid (11 mg, 14% yield): ¹H NMR (400 MHz, DMSO-D₆) δ 4.44 (d, J=5.6 Hz, 2 H) 6.94 (t, J=5.9 Hz, 1 H) 7.08 (dd, J=7.7, 1.6 Hz, 1 H) 7.11-7.17 (m, 2 H) 7.18 (t, J=2.0 Hz, 1 H) 7.35 (t, J=8.1 Hz, 2 H) 7.49-7.56 (m, 2 H) 7.60 (dd, J=4.8, 3.0 Hz, 1 H) 7.77 (d, J=7.8 Hz, 1 H) 7.92 (dd, J=3.0,1.3 Hz, 1 H) 8.43 (s, 1 H) 8.46 (d, J=4.6 Hz, 1 H) 8.61 (s, 1 H) 9.38 (s, 1 H); HRMS (ESI+) calcd for C₂₆H₁₉ClN₅S (MH+) 468.1044, found 468.1046.

### Example 101

### 8-bromo-4-(3-chloro-4-fluorophenylamino)-6-(1-oxypyridin-2-ylmethylamino)quinoline-3-carbonitrile

Following the procedure described above in Example 4, 6-amino-8-bromo-4-(3-chloro-4-fluorophenylamino)quinoline-3-carbonitrile (0.600 g, 1.53 mmol) was reacted with methoxy-(1-oxypyridin-2-yl)methanol (0.238 g, 1.53 mmol) and NaCNBH₃ (64 mg, 1.0 mmol) in 18 mL THF and 6 mL MeOH. After the reaction mixture had stirred overnight, the bright yellow precipitate was collected by suction filtration, washed with methanol and dried under vacuum to give pure product as a bright yellow powder (0.352 g, 46% yield): ¹H NMR (400 MHz, DMSO-D₆) δ 4.58 (d, J=6.1 Hz, 2 H) 7.04 (t, J=6.3 Hz, 1 H) 7.17 (s, 1 H) 7.20-7.44 (m, 5 H) 7.49 (dd, J=6.6, 2.5 Hz, 1 H) 7.82 (d, J=1.8 Hz, 1 H) 8.31 (d, J=6.1 Hz, 1 H) 8.37 (s, 1 H) 9.46 (s, 1 H); HRMS (ESI+) calcd for C₂₂H₁₅BrClFN₅O (MH+) 498.0127, found 498.0108.

### Example 102

### 4-(3-chloro-4-fluorophenylamino)-8-(furan-3-yl)-6-(pyridin-3-ylmethylamino)quinoline-3-carbonitrile

Following the procedure described above in Example 100, 8-bromo-4-(3-chloro-4-fluorophenylamino)-6-(pyridin-3-ylmethylamino)quinoline-3-carbonitrile (0.200 g, 0.414 mmol, prepared as described in Example 77 above) was reacted with with furan-3-boronic acid (56 mg, 0.50 mmol), Pd(PPh₃)₂Cl₂ (15 mg, 0.021 mmol) and Na₂CO₃ (53 mg, 0.50 mmol) for 15 minutes at 130° C. The crude product was purified by preparative HPLC and lyophilized to give a golden-brown solid (41 mg, 21% yield): ¹H NMR (400 MHz, DMSO-D₆) δ 4.45 (d, J=5.8 Hz, 2 H) 6.85 (t, J=6.2 Hz, 1 H) 6.96 (s, 1 H) 7.15 (d, J=2.3 Hz, 1 H) 7.18-7.24 (m, 1 H) 7.32-7.45 (m, 3 H) 7.60 (d, J=2.0 Hz, 1 H) 7.74-7.82 (m, 2 H) 8.41 (s, 1 H) 8.46 (d, J=4.3 Hz, 1 H) 8.54 (s, 1 H) 8.63 (s, 1 H) 9.36 (s, 1 H); HRMS (ESI+) calcd for C₂₆H₁₈ClFN₅O (MH+) 470.1179, found 470.1186.

### Example 103

### 2-(4-(3-chloro-4-fluorophenylamino)-3-cyanoquinolin-6-ylamino)-N,N-dimethylacetamide

Step 1: Following the procedure described above in Example 76, 4-chloro-6-nitroquinoline-3-carbonitrile (2.50 g, 5.35 mmol) was reacted with 3-chloro-4-fluoroaniline (1.71 g, 11.8 mmol), in 2 batches, each in 5 mL EtOH, except that CHCl₃ was used instead of EtOAc for the work-up. The 2 batches were worked up together, and the crude product 6-amino-4-(3-chloro-4-fluorophenylamino)quinoline-3-carbonitrile purified by flash chromatography over silica gel (gradient elution, 4-6% MeOH in CH₂Cl₂) to give a brown solid (1.36 g, 81% yield): ¹H NMR (400 MHz, DMSO-D₆) δ 5.76 (s, 2 H) 7.11-7.16 (m, 2 H) 7.24 (dd, J=9.0, 2.4 Hz, 1 H) 7.32-7.40 (m, 2 H) 7.69 (d, J=8.8 Hz, 1 H) 8.32 (s, 1 H) 9.34 (s, 1 H).

Step 2: Following the procedure described above in Example 4, 6-amino-4-(3-chloro-4-fluorophenylamino)quinoline-3-carbonitrile (1.26 g, 4.03 mmol) was reacted with a 50 wt% solution of glyoxylic acid in water (0.44 mL, 0.30 g, 4.0 mmol) and NaCNBH₃ (0.170 g, 2.70 mmol), in 40 mL THF and 15 mL MeOH. The yellow precipitate was collected out of the reaction mixture by suction filtration, washing with MeOH, and dried under vacuum to give the product 2-(4-(3-chloro-4-fluorophenylamino)-3-cyanoquinolin-6-ylamino)acetic acid as a yellow-orange powder (0.420 g, 28% yield): ¹H NMR (400 MHz, DMSO-D₆) δ 3.95 (s, 2 H) 6.50 (br s, 1 H) 7.09 (d, J=2.0 Hz, 1 H) 7.21-7.31 (m, 1 H) 7.35-7.46 (m, 2 H) 7.49 (dd, J=6.3, 2.5 Hz, 1 H) 7.68 (d, J=8.8 Hz, 1 H) 8.29 (s, 1 H) 9.34 (s, 1 H).

Step 3: Following the procedure described above in Example 68, 2-(4-(3-chloro-4-fluorophenylamino)-3-cyanoquinolin-6-ylamino)acetic acid (0.100 g, 0.270 mmol) was reacted with dimethylamine hydrochloride (24 mg, 0.30 mmol), BOP reagent (0.131 g, 0.297 mmol) and 4-methylmorpholine (0.065 mL, 60 mg, 0.59 mmol) in 3 mL DMF. Product was obtained as a brownish-yellow powder (62 mg, 58% yield): ¹H NMR (400 MHz, DMSO-D₆) δ 2.89 (s, 3 H) 3.02 (s, 3 H) 3.97 (s, 2 H) 6.26 (s, 1 H) 7.04 (s, 1 H) 7.24-7.32 (m, 1 H) 7.40-7.56 (m, 3 H) 7.69 (d, J=9.4 Hz, 1 H) 8.34 (s,1 H) 9.34 (s, 1 H); HRMS (ESI+) calcd for C₂₀H₁₈ClFN₅O (MH+) 398.1179, found 398.1173.

### Example 104

### 8-bromo-4-(tert-butylamino)-6-(1-oxypyridin-2-ylmethylamino)quinoline-3-carbonitrile

Following the procedure described above in Example 4, 6-amino-8-bromo-4-(tert-butylamino)quinoline-3-carbonitrile (0.213 g, 0.667 mmol) was reacted with methoxy-(1-oxypyridin-2-yl)methanol (0.104 g, 0.667 mmol) and NaCNBH₃ (28 mg, 0.45 mmol) in 6 mL THF and 2 mL MeOH. The crude product was purified twice by preparative HPLC and once by flash chromatography over silica gel (gradient elution, 1-10% MeOH in CH₂Cl₂), then lyophilized, to give a beige solid (26 mg, 9.2% yield): ¹H NMR (400 MHz, DMSO-D₆) δ 1.33 (s, 9 H) 4.62 (d, J=6.1 Hz, 2 H) 6.12 (s, 1 H) 6.99 (d, J=2.0 Hz, 1 H) 7.09 (t, J=6.1 Hz, 1 H) 7.24-7.39 (m, 3 H) 7.73 (s, 1 H) 8.33 (d, J=6.6 Hz, 1 H) 8.43 (s,1 H); HRMS (ESI+) calcd for C₂₀H₂₁BrN₅O (MH+) 426.0924, found 426.0929.

### Example 105

### 8-bromo-4-(3-chloro-4-fluorophenylamino)-6-((2-methyl-1H-imidazol-5-yl)methylamino)quinoline-3-carbonitrile

Following the procedure described above in Example 4, 6-amino-8-bromo-4-(3-chloro-4-fluorophenylamino)quinoline-3-carbonitrile (0.300 g, 0.766 mmol, prepared as described in Example 78 above) was reacted with 2-methyl-1H-imidazole-5-carbaldehyde (84 mg, 0.77 mmol) and NaCNBH₃ (32 mg, 0.51 mmol) in 9 mL THF and 3 mL MeOH. The crude product was purified by preparative HPLC and lyophilized to give a bright yellow solid (0.238 g, 64% yield): ¹H NMR (400 MHz, DMSO-D₆) δ 2.23 (s, 3 H) 4.16 (d, J=5.1 Hz, 2 H) 6.64 (t, J=4.8 Hz, 1 H) 6.87 (s, 1 H) 7.21 (d, J=1.8 Hz, 1 H) 7.25-7.32 (m, 1 H) 7.44 (t, J=9.1 Hz, 1 H) 7.52 (dd, J=6.6, 2.5 Hz, 1 H) 7.78 (d, J=1.5 Hz, 1 H) 8.36 (s, 1 H) 9.47 (s, 1 H) 11.84 (br s, 1 H); HRMS (ESI+) calcd for C₂₁H₁₆BrClFN₆O (MH+) 485.0287, found 485.0290.

### Example 106

### 8-bromo-4-(3-chloro-4-fluorophenylamino)-6-((2-phenyl-1H-imidazol-5-yl)methylamino)quinoline-3-carbonitrile

Following the procedure described above in Example 4, 6-amino-8-bromo-4-(3-chloro-4-fluorophenylamino)quinoline-3-carbonitrile (0.300 g, 0.766 mmol, prepared as described in Example 78 above) was reacted with 2-phenyl-1H-imidazole-5-carbaldehyde (0.132 g, 0.766 mmol) and NaCNBH₃ (32 mg, 0.51 mmol) in 9 mL THF and 3 mL MeOH. The crude product was purified by trituration with boiling EtOH, and a second crop of crystals collected from the filtrate, to give a yellow solid (0.206 g, 49% yield): ¹H NMR (400 MHz, DMSO-D₆) δ 4.30 (d, J=5.8 Hz, 2 H) 6.77 (t, J=5.3 Hz, 1 H) 7.20 (s, 1 H) 7.26-7.35 (m, 3 H) 7.37-7.47 (m, 3 H) 7.53 (dd, J=6.6, 2.8 Hz, 1 H) 7.81 (s, 1 H) 7.90 (d, J=7.3 Hz, 2 H) 8.37 (s, 1 H) 9.49 (s, 1 H) 11.94 (s, 0.5 H) 12.40 (s, 0.5 H); HRMS (ESI+) calcd for C₂₆H₁₈BrClFN₆ (MH+) 547.0444, found 547.0457.

### Example 107

### 8-bromo-6-((2-butyl-1H-imidazol-5-yl)methylamino)-4-(3-chloro-4-fluorophenylamino)quinoline-3-carbonitrile

Step 1: The procedure described by R. Paul and J. Menschik (J. Heterocyclic Chem. 1979, 16, 277-282) was followed. In a 50 mL round-bottomed flask, (2-butyl-1H-imidazol-5-yl)methanol (5.00 g, 32.4 mmol) was taken up in 5 mL concentrated nitric acid. The open flask was heated in an oil bath at 100° C. until brown fumes issued from its mouth, lifted out of the oil bath briefly to ensure that the reaction did not become too vigorous, and then, upon calming, returned to the oil bath and heated until the evolution of brown fumes ceased. The reaction mixture was then cooled to RT and neutralized with saturated Na₂CO₃, and the off-white precipitate collected, rinsed with water, and dried under vacuum to give pure product 2-butyl-1H-imidazole-5-carbaldehyde (2.64 g, 53% yield): ¹H NMR (400 MHz, DMSO-D₆) δ 0.87 (t, J=7.5 Hz, 3 H) 1.28 (br s, 2 H) 1.63 (quint, 2 H) 7.73 (s, 0.5 H) 7.90 (s, 0.5 H) 9.56 (s, 0.5 H) 9.64 (s, 0.5 H) 12.50 (s, 0.5 H) 12.89 (s, 0.5 H).

Step 2: Following the procedure described above in Example 76, 6-amino-8-bromo-4-(3-chloro-4-fluorophenylamino)quinoline-3-carbonitrile (0.300 g, 0.766 mmol) was reacted with 2-butyl-1H-imidazole-5-carbaldehyde (0.117 g, 0.766 mmol) and NaCNBH₃ (32 mg, 0.51 mmol) in 9 mL THF and 3 mL MeOH. The crude product was recrystallized from MeCN to give a yellow solid (0.193 g, 48% yield): ¹H NMR (400 MHz, DMSO-D₆) δ 0.87 (t, J=7.3 Hz, 3 H) 1.30 (sext, 2 H) 1.60 (quint, 2 H) 2.53-2.61 (m, 2 H) 4.19 (d, J=4.8 Hz, 2 H) 6.64 (t, J=5.2 Hz, 1 H) 6.88 (s, 1 H) 7.24 (d, J=2.0 Hz, 1 H) 7.28 (ddd, J=8.9, 4.1, 2.7 Hz, 1 H) 7.44 (t, J=9.1 Hz, 1 H) 7.52 (dd, J=6.6, 2.8 Hz, 1 H) 7.79 (d, J=2.0 Hz, 1 H) 8.37 (s, 1 H) 9.48 (s, 1 H) 11.59 (s,1 H); HRMS (ESI+) calcd for C₂₄H₂₂BrClFN₆ (MH+) 527.0757, found 527.0761.

### Example 108

### 2-(4-(3-chloro-4-fluorophenylamino)-3-cyanoquinolin-6-ylamino)-N-methylacetamide

Following the procedure described above in Example 103, 2-(4-(3-chloro-4-fluorophenylamino)-3-cyanoquinolin-6-ylamino)acetic acid (0.150 g, 0.405 mmol) was reacted with methylamine hydrochloride (30 mg, 0.45 mmol) in the presence of BOP reagent (0.197 g, 0.445 mmol) and 4-methylmorpholine (0.098 mL, 90 mg, 0.89 mmol), in 3 mL DMF. Product was isolated as a bright yellow powder (0.113 g, 73% yield): ¹H NMR (400 MHz, DMSO-D₆) δ 2.60 (d, J=4.3 Hz, 3 H) 3.80 (d, J=5.6 Hz, 2 H) 6.56 (s, 1 H) 7.08 (s, 1 H) 7.18-7.49 (m, 4 H) 7.71 (d, J=8.6 Hz, 1 H) 7.83 (s, 1 H) 8.33 (s, 1 H) 9.37 (s, 1 H); HRMS (ESI+) calcd for C₁₉H₁₆ClFN₅O (MH+) 384.1022, found 384.1019.

### Example 109

### (E)-4-(3-chloro-4-fluorophenylamino)-8-(prop-1-enyl)-6-(pyridin-3-ylmethylamino)quinoline-3-carbonitrile

In a flame-dried, crimp-sealed microwave vial, under an inert atmosphere, 8-bromo-4-(3-chloro-4-fluorophenylamino)-6-(pyridin-3-ylmethylamino)quinoline-3-carbonitrile (0.100 g, 0.207 mmol, prepared as described in Example 77) and Pd(PPh₃)₄ (24 mg, 0.021 mmol) were taken up in 4 mL anhydrous THF. A flame-dried 25 mL round-bottomed flask, under an inert atmosphere, was charged with a 0.5M THF solution of 1-propenylmagnesium bromide (1.2 mL, 0.62 mmol). A 0.5M THF solution of zinc chloride (1.2 mL, 0.62 mmol) was then added, and the mixture stirred for 5 minutes at RT. It was then transferred by syringe to the microwave tube. The yellow suspension immediately became a clear, dark red solution. The vial and its contents were heated in a microwave reactor at 110° C. for 5 minutes, until LC-MS analysis showed that most of the bromide starting material had been consumed. The contents of the vial were then partitioned between EtOAc and saturated NH₄Cl, the aqueous layer extracted twice more with EtOAc, and the combined organic layers washed with brine, dried over anhydrous MgSO₄, filtered, and evaporated. The crude product was purified by preparative HPLC and lyophilized to give a golden yellow powder (16 mg, 18% yield): ¹H NMR (400 MHz, DMSO-D₆) δ 1.94 (dd, J=6.6, 1.8 Hz, 3 H) 4.44 (d, J=5.6 Hz, 2 H) 6.24-6.41 (m, 1 H) 6.81 (t, J=5.9 Hz, 1 H) 7.11 (d, J=2.3 Hz, 1 H) 7.19 (ddd, J=8.7, 4.2, 2.8 Hz, 1 H) 7.31-7.53 (m, 5 H) 7.77 (d, J=7.8 Hz, 1 H) 8.36 (s, 1 H) 8.46 (d, J=4.3 Hz, 1 H) 8.61 (s, 1 H) 9.28 (s, 1 H); HRMS (ESI+) calcd for C₂₅H₂₀ClFN₅ (MH+) 444.1386, found 444.1379.

### Example 110

### Methyl 5-((8-bromo-4-(3-chloro-4-fluorophenylamino)-3-cyanoquinolin-6-ylamino)methyl)-1H-imidazole-4-carboxylate

Step 1: In a microwave vial, methyl 5-hydroxymethyl-1H-imidazole-4-carboxylate (0.200 g, 1.28 mmol) was taken up in 2.5 mL each CH₂Cl₂ and 1,4-dioxane, and activated MnO₂ (0.95 g, 11 mmol) was added. The vial was crimp-sealed and heated in a microwave reactor at 140° C. for 5 minutes, until LC-MS analysis showed complete disappearance of starting material. The contents of the vial were then rinsed into a 1 L Erlenmeyer flask and stirred with 400 mL MeOH for 30 minutes. The suspension was then filtered to remove MnO₂, and the filtrate dried over anhydrous MgSO₄, filtered a second time, and evaporated to give product methyl 5-formyl-1H-imidazole-4-carboxylate of sufficient purity to be used in the next step (0.163 g, 83% yield): ¹H NMR (400 MHz, DMSO-D₆) δ 3.88 (s, 3 H) 8.06 (s, 1 H) 10.22 (s, 1 H) 13.76 (s, 1 H); HRMS (ESI+) calcd for C₆H₇N₂O₃ (MH+) 155.0451, found 155.0450.

Step 2: Following the procedure described above in Example 76, 6-amino-8-bromo-4-(3-chloro-4-fluorophenylamino)quinoline-3-carbonitrile (0.400 g, 1.02 mmol, prepared as described abovie in Example 78) was reacted with with methyl 5-formyl-1H-imidazole-4-carboxylate (157 mg, 1.02 mmol) and NaCNBH₃ (43 mg, 0.68 mmol) in 12 mL THF and 4 mL MeOH. The crude product was purified by preparative HPLC and lyophilized to give a bright yellow powder (0.18 g, 33% yield): ¹H NMR (400 MHz, DMSO-D₆) 6 3.76 (br s, 3 H) 4.50 (br s,1 H) 4.62 (br s, 1 H) 6.65 (br s, 0.5 H) 6.74 (br s, 0.5 H) 7.20-7.31 (m, 2 H) 7.43 (t, J=9.0 Hz, 1 H) 7.50 (dd, J=6.4, 2.7 Hz, 1 H) 7.63-7.88 (m, 2 H) 8.39 (s, 1 H) 9.45 (br s, 1 H) (br s, 0.5 H) 13.09 (br s, 0.5 H); HRMS (ESI+) calcd for C₂₂H₁₆BrClFN₆O₂ (MH+) 529.0185, found 529.0186.

### Example 111

### 8-bromo-4-(3-chloro-4-fluorophenylamino)-6-((4-phenyl-1H-imidazol-5-yl)methylamino)quinoline-3-carbonitrile

Step 1: The procedure described by Y. Hayashi et al. (J. Org. Chem. 2000, 65, 8402-8405) was followed. In a 250 mL round-bottomed flask fitted with an addition funnel, ethyl benzoylacetate (9.0 mL, 10 g, 52 mmol) was dissolved in 40 mL CHCl₃ and cooled to 0° C. in an ice bath. Sulfuryl chloride (4.4 mL, 7.4 g, 55 mmol) was then added dropwise via the addition funnel. After the addition was complete, the ice bath was removed and the solution allowed to stir for 1 hour at RT. It was then heated at reflux for 2 hours. Upon cooling to RT, the cloudy yellow solution was washed successively with water, saturated NaHCO₃, water and brine, dried over anhydrous MgSO₄, filtered, and evaporated to give the product ethyl 2-chloro-3-oxo-3-phenylpropanoate as a golden-yellow oil of sufficient purity to be used in the next step (12.7 g, 108% yield): ¹H NMR (400 MHz, CDCl₃) δ 1.24 (t, J=7.2 Hz, 3 H) 4.28 (q, J=7.2 Hz, 2 H) 5.59 (s, 1 H) 7.50 (t, J=7.7 Hz, 2 H) 7.63 (t, J=7.5 Hz, 1 H) 7.99 (d, J=7.3 Hz, 2 H).

Step 2: A modification of the procedures described by Y. Hayashi et al. (J. Org. Chem. 2000, 65, 8402-8405) and G. Durant et al. (U.S. Pat. No. 4024271) was followed. A 250 mL round-bottomed flask fitted with a condenser was charged with ethyl 2-chloro-3-oxo-3-phenylpropanoate (6.7 g, 30 mmol), formamide (12 mL, 13 g, 0.30 mol) and water (1.1 mL, 1.1 g, 59 mmol), and heated at 195° C. until LC-MS analysis showed desired product as the major component. The reaction mixture was then cooled to RT and partitioned between CHCl₃ and saturated Na₂CO₃. The aqueous layer was extracted twice more with CHCl₃, and the combined organic layers washed twice with saturated Na₂CO₃ and twice with brine, then dried over anhydrous MgSO₄, filtered and evaporated. The crude product was purified by flash chromatography over silica gel (5% MeOH in CH₂Cl₂) to give the product ethyl 4-phenyl-1H-imidazole-5-carboxylate as an off-white solid (0.833 g, 13% yield): ¹H NMR (400 MHz, DMSO-D₆) δ 1.17 (t, J=7.1 Hz, 1.2 H) 1.25 (t, J=7.1 Hz, 1.8 H) 4.14 (q, J=7.1 Hz, 0.8 H) 4.24 (q, J=7.2 Hz, 1.2 H) 7.28-7.47 (m, 3.6 H) 7.61 (d, J=7.1 Hz, 1 H) 7.79 (s, 0.4 H) 7.83-7.92 (m, 2 H) 12.86 (br s, 0.4 H) 13.02 (br s, 0.6

Step 3: In a flame-dried 100 mL round-bottomed flask under an inert atmosphere, ethyl 4-phenyl-1H-imidazole-5-carboxylate (1.14 g, 5.26 mmol) was taken up in 25 mL anhydrous THF and cooled to 0° C. in an ice bath. A 1.0 M solution of lithium aluminum hydride in THF (5.3 mL, 5.3 mmol) was then added slowly via syringe. After the addition was complete, the ice bath was removed, and the reaction mixture allowed to warm to RT over 30 minutes. The reaction was then cooled back to 0° C. and quenched by addition of 5 mL saturated Na₂SO₄. This was followed by addition of enough 1 M HCl to bring the pH of the solution down to 8. The white precipitate was then filtered off, washing with copious amounts of EtOAc. The filtrate was dried over anhydrous MgSO₄, filtered and evaporated to give the product (4-phenyl-1H-imidazol-5-yl)methanol as a waxy yellow solid of sufficient purity to be used in the next step (0.765 g, 83% yield): ¹H NMR (400 MHz, DMSO-D₆) δ 4.54 (d, J=4.3 Hz, 2 H) 5.19 (br s,1 H) 7.24 (t, J=7.3 Hz, 1 H) 7.39 (t, J=7.7 Hz, 2 H) 7.62 (s,1 H) 7.68 (d, J=7.1 Hz, 2 H) 12.24 (br s, 1 H).

Step 4: Following the procedure described above in Example 110, (4-phenyl-1H-imidazol-5-yl)methanol (0.400 g, 2.30 mmol) was reacted with activated manganese dioxide (0.400 g, 4.60 mmol). The crude product 4-phenyl-1H-imidazole-5-carbaldehyde obtained was of sufficient purity to be used directly in the next step (0.538 g, 136% yield): ¹H NMR (400 MHz, DMSO-D₆) δ 7.40-7.53 (m, 3 H) 7.83 (d, J=7.1 Hz, 2 H) 8.03 (s, 1 H) 9.86 (s, 1 H) 13.29 (s, 1 H).

Step 5: Following the procedure described above in Example 4, 6-amino-8-bromo-4-(3-chloro-4-fluorophenylamino)quinoline-3-carbonitrile (0.300 g, 0.766 mmol, prepared as described above in Example 78) was reacted with 4-phenyl-1H-imidazole-5-carbaldehyde (132 mg, 0.766 mmol) and NaCNBH₃ (32 mg, 0.51 mmol) in 9 mL THF and 3 mL MeOH. The reaction mixture was allowed to stir overnight after addition of NaCNBH₃, but LC-MS analysis showed that more 6-aminoquinoline than product was present. Additional aldehyde (132 mg) and NaCNBH₃ were added, and the reaction was stirred for an additional day. Solvent was then removed under reduced pressure, and the crude product purified by preparative HPLC and lyophilized, to give a bright yellow solid (62 mg, 15% yield): ¹H NMR (400 MHz, DMSO-D₆) δ m 4.33 (dd, J=49.4, 3.9 Hz, 2 H) 6.75 (s, 0.5 H) 6.95 (s, 0.5 H) 7.18-7.90 (m, 11 H) 8.41 (d, J=8.3 Hz, 1 H) 9.45 (s, 1 H) 12.43 (s, 0.5 H) 12.53 (s, 0.5 H); HRMS (ESI+) calcd for C₂₆H₁₈BrClFN₆ (MH+) 547.0444, found 547.0451.

### Example 112

### 8-bromo-4-(3-chloro-4-fluorophenylamino)-6-((4-ethyl-1H-imidazol-5-yl)methylamino)quinoline-3-carbonitrile

Step 1: Following the procedure described above in Example 111, ethyl propionylacetate (9.9 mL, 10 g, 69 mmol) was reacted with sulfuryl chloride (5.9 mL, 9.8 g, 73 mmol) in 50 mL CHCl₃, to give pure product ethyl 2-chloro-3-oxopentanoate as a colorless oil (9.90 g, 80% yield): ¹H NMR (400 MHz, CDCl₃) δ 1.11 (t, J=7.2 Hz, 3 H) 1.30 (t, J=7.1 Hz, 3 H) 2.73 (dq, J=7.2, 2.5Hz, 2 H) 4.27 (q, J=7.2 Hz, 2 H) 4.78 (s, 1 H).

Step 2: Following the procedure described above in Example 111, ethyl 2-chloro-3-oxopentanoate (8.90 g, 49.8 mmol) with formamide (20 mL, 22 g, 0.50 mol) and water (1.8 mL, 1.8 g, 100 mmol). To work up the reaction, 50 mL 1 M HCl was added to the cooled, dark brown solution, and it was then heated to its boiling point, treated with activated charcoal, and filtered while hot. The clear reddish-golden brown solution was then acidified with additional 1 M HCl to ph 1, then basified with concentrated NH₄OH and extracted with 3 portions of CHCl₃. The combined organic extracts were dried over anhydrous MgSO₄, filtered, and evaporated to give an oily yellow solid. This was purified by flash chromatography over silica gel (gradient elution, 1-10% MeOH in CH₂Cl₂) to give the product ethyl 4-ethyl-1H-imidazole-5-carboxylate as a white crystalline solid (0.641 g, 7.6% yield): ¹H NMR (400 MHz, DMSO-D₆) δ 1.15 (t, J=7.5 Hz, 3 H) 1.21-1.33 (m, 3 H) 2.78 (q, J=7.6 Hz, 0.68 H) 2.88 (q, J=7.6 Hz, 1.32 H) 4.19 (q, J=7.1 Hz, 1.32 H) 4.25 (q, J=7.2 Hz, 0.68 H) 7.58 (s, 0.66 H) 7.68 (s, 0.34 H) 12.42 (br s, 0.66 H) 12.72 (br s, 0.34 H).

Step 3: Following the procedure described above in Example 111, ethyl 4-ethyl-1H-imidazole-5-carboxylate (0.641 g, 3.81 mmol) was reacted with a 1.0M THF solution of lithium aluminum hydride (3.8 mL, 3.8 mmol) in 20 mL THF. After quenching the reaction mixture with saturated Na₂SO₄, the white precipitate was filtered off, washing with copious amounts of EtOAc, and the filtrate dried over anhydrous MgSO₄, filtered and evaporated to give a product (4-ethyl-1H-imidazol-5-yl)methanol of sufficient purity to be used directly in the next step (0.471 g, 98% yield): ¹H NMR (400 MHz, DMSO-D₆) δ 1.11 (t, J=7.6 Hz, 3 H) 2.46-2.53 (m, 2 H) 4.32 (s, 2 H) 4.66 (s, 1 H) 7.39 (s, 1 H) 11.67 (s, 1 H).

Step 4: Following the procedure described above in Example 110, (4-ethyl-1H-imidazol-5-yl)methanol (0.471 g, 3.73 mmol) was reacted with activated manganese dioxide (0.973 g, 11.2 mmol) to give the product 4-ethyl-1H-imidazole-5-carbaldehyde as an oily brown solid of sufficient purity to be used directly in the next step (0.386 g, 83% yield): ¹H NMR (400 MHz, DMSO-D₆) δ 1.19 (br s, 3 H) 2.86 (br s, 2 H) 7.73 (br s,1 H) 9.78 (br s,1 H).

Step 5: Following the procedure described above in Example 4, 6-amino-8-bromo-4-(3-chloro-4-fluorophenylamino)quinoline-3-carbonitrile (0.300 g, 0.766 mmol, prepared as described above in Example 78) was reacted with 4-ethyl-1H-imidazole-5-carbaldehyde (95 mg, 0.77 mmol) and NaCNBH₃ (32 mg, 0.51 mmol) in 9 mL THF and 3 mL MeOH. The crude product was purified by preparative HPLC, and lyophilized to give a fluffy bright yellow solid (111 mg, 29% yield): ¹H NMR (400 MHz, DMSO-D₆) δ 1.11 (t, J=7.5 Hz, 3 H) 2.57 (br s, 2 H) 4.14 (br s, 2 H) 6.51 (br s, 1 H) 7.23 (s, 1 H) 7.25-7.35 (m, 1 H) 7.44 (t, J=9.1 Hz, 1 H) 7.48-7.57 (m, 2 H) 7.81 (s, 1 H) 8.39 (s, 1 H) 9.46 (s, 1 H) 11.82 (br s,1 H); HRMS (ESI+) calcd for C₂₂H₁₈BrClFN₆ (MH+) 499.0444, found 499.0453.

### Example 113

### 8-bromo-4-(3-chloro-4-fluorophenylamino)-6-((1,5-dimethyl-1H-imidazol-4-yl)methylamino)quinoline-3-carbonitrile

Following the procedure described above in Example 4, 6-amino-8-bromo-4-(3-chloro-4-fluorophenylamino)quinoline-3-carbonitrile (88 mg, 0.226 mmol, prepared as described in Example 78) was reacted with 1,5-dimethyl-1H-imidazole-4-carbaldehyde (28 mg, 0.27 mmol) and NaCNBH₃ (10 mg, 0.15 mmol) in 3 mL THF and 1 mL MeOH. The crude product was purified by preparative HPLC and lyophilized to give a fluffy bright yellow solid (36 mg, 32% yield): ¹H NMR (400 MHz, DMSO-D₆) δ 2.16 (s, 3 H) 3.51 (s, 3 H) 4.12 (d, J=4.6 Hz, 2 H) 6.50 (t, J=4.7 Hz, 1 H) 7.23 (d, J=1.8 Hz, 1 H) 7.25-7.31 (m, 1 H) 7.44 (t, J=9.0 Hz, 1 H) 7.48-7.54 (m, 2 H) 7.80 (d, J=2.0 Hz, 1 H) 8.38 (s, 1 H) 9.46 (s, 1 H); HRMS (ESI+) calcd for C₂₂H₁₈BrClFN₆ (MH+) 499.0444, found 499.0455.

### Example 114

### 8-bromo-4-(3-chloro-4-fluorophenylamino)-6-((4-(trifluoromethyl)-1H-imidazol-5-yl)methylamino)quinoline-3-carbonitrile

Step 1: Following the procedure described above in Example 111, ethyl 2-chloro-4,4,4-trifluoroacetoacetate (50 g, 0.23 mol) was reacted with formamide (91.0 mL, 103 g, 2.29 mol) and water (8.3 mL, 8.3 g, 0.46 mol). The reaction mixture, which turned into a brown sludge, was worked up by pouring into ice water, diluting to 300 mL, and collecting the precipitate by suction filtration, washing with water and drying under vacuum. Pure product ethyl 4-(trifluoromethyl)-1H-imidazole-5-carboxylate was obtained as a dark brown powder (9.90 g, 21% yield): ¹H NMR (400 MHz, DMSO-D₆) δ 1.30 (t, J=7.1 Hz, 3 H) 4.33 (q, J=7.1 Hz, 2 H) 8.01 (s, 1 H) 13.89 (s, 1 H); HRMS (ESI+) calcd for C₇H₈F₃N₂O₂ (MH+) 209.0533, found 209.0533.

Step 2: Following the procedure described above in Example 111, ethyl 4-(trifluoromethyl)-1H-imidazole-5-carboxylate (1.00 g, 4.80 mmol) with a 1.0M THF solution of lithium aluminum hydride (4.8 mL, 4.8 mmol) in 20 mL THF. After filtering off the precipitate during the work-up, the filtrate was diluted with an equal volume of MeOH to dissolve the dark oil clinging to the bottom of the flask, dried over anhydrous MgSO₄, filtered, and evaporated to give the product (4-(trifluoromethyl)-1H-imidazol-5-yl)methanol as an oily, light orange solid of sufficient purity to be used directly in the next step (0.745 g, 94% yield): ¹H NMR (400 MHz, DMSO-D₆) δ 4.53 (d, J=1.5 Hz, 2 H) 7.70 (s, 1 H) 13.07 (s, 1 H).

Step 3: Following the procedure described above in Example 110, (4-(trifluoromethyl)-1H-imidazol-5-yl)methanol (0.500 g, 3.01 mmol) was reacted with activated manganese dioxide (0.79 g, 9.0 mmol) to give a product 4-(trifluoromethyl)-1H-imidazole-5-carbaldehyde of sufficient purity to be used directly in the next step (0.573 g, 116% yield): ¹H NMR (400 MHz, DMSO-D₆) 6 7.88 (br s, 1 H) 9.83 (br s, 1 H).

Step 4: Following the procedure described above in Example 4, 6-amino-8-bromo-4-(3-chloro-4-fluorophenylamino)quinoline-3-carbonitrile (0.300 g, 0.766 mmol prepared as described above in Example 78) was reacted with 4-(trifluoromethyl)-1H-imidazole-5-carbaldehyde (126 mg, 0.766 mmol) and NaCNBH₃ (32 mg, 0.51 mmol) in 9 mL THF and 3 mL MeOH. After 1 day, LC-MS analysis showed that very little product was present, so additional aldehyde (126 mg) and NaCNBH₃ (32 mg) were added. After another day, there was still more 6-aminoquinoline than desired product, so additional aldehyde (126 mg) and NaCNBH₃ (32 mg) were once again added, and the reaction was allowed to stir for 3 days. Solvent was then removed under reduced pressure, and the crude product purified twice by preparative HPLC, and lyophilized to give a brownish-yellow powder (22 mg, 5.2% yield): ¹H NMR (400 MHz, DMSO-D₆) δ 4.41 (d, J=4.0 Hz, 2 H) 6.76-6.86 (m, 1 H) 7.23-7.33 (m, 2 H) 7.45 (t, J=9.0 Hz, 1 H) 7.53 (dd, J=6.8, 2.5 Hz, 1 H) 7.73 (d, J=2.0 Hz, 1 H) 7.83 (s, 1 H) 8.42 (s, 1 H) 9.47 (br s,1 H) 12.92 (br s,1 H); HRMS (ESI+) calcd for C₂₁H₁₃BrClF₄N₆ (MH+) 539.0004, found 539.0014.

### Example 115

### 8-bromo-4-(3-chloro-4-fluorophenylamino)-6-((4-isopropyl-1H-imidazol-5-yl)methylamino)quinoline-3-carbonitrile

Step 1: Following the procedure described above in Example 111, ethyl isobutyrylacetate (10.2 mL, 10.0 g, 63.2 mmol) was reacted with sulfuryl chloride (5.3 mL, 9.0 g, 66 mmol) in 50 mL CHCl₃, to give product ethyl 2-chloro-4-methyl-3-oxopentanoate of sufficient purity to be used directly in the next step (12.2 g, 94% yield): ¹H NMR (400 MHz, CDCl₃) δ 1.17 (dd, J=9.1, 6.8 Hz, 6 H) 1.30 (t, J=6.8 Hz, 3 H) 2.99-3.16 (m, 1 H) 4.24-4.33 (m, 2 H) 4.92 (s, 1 H).

Step 2: Following the procedure described above in Example 111, ethyl 2-chloro-4-methyl-3-oxo-pentanoate (12.2 g, 63.3 mol) was reacted with formamide (25 mL, 29 g, 0.63 mol) and water (2.3 mL, 2.3 g, 0.13 mol). The crude product was purified by flash chromatography over silica gel (4-6% MeOH in CH₂Cl₂) to give product ethyl 4-isopropyl-1H-imidazole-5-carboxylate of sufficient purity to be used in the next step (0.558 g, 4.8% yield): ¹H NMR (400 MHz, DMSO-D₆) δ 1.20 (d, J=7.1 Hz, 6 H) 1.23-1.33 (m, 3 H) 3.44-3.57 (m, 0.35 H) 3.65-3.79 (m, 0.65 H) 4.12-4.33 (m, 2 H) 7.58 (s, 0.65 H) 7.66 (s, 0.35 H) 12.39 (br s, 0.65 H) 12.67 (br s, 0.35 H).

Step 3: Following the procedure described above in Example 112, ethyl 4-isopropyl-1H-imidazole-5-carboxylate (0.558 g, 3.06 mmol) was reacted with a 1.0M THF solution of lithium aluminum hydride (3.1 mL, 3.1 mmol) in 20 mL THF. Work-up gave product (4-isopropyl-1H-imidazol-5-yl)methanol of sufficient purity to be used directly in the next step (0.397 g, 92% yield): ¹H NMR (400 MHz, DMSO-D₆) δ 1.15 (d, J=6.8 Hz, 6 H) 2.78-3.12 (m, 1 H) 4.33 (s, 2 H) 4.66 (br s,1 H) 7.38 (s, 1 H) 11.66 (br s,1 H).

Step 4: Following the procedure described above in Example 110, (4-isopropyl-1H-imidazol-5-yl)methanol (0.217 g, 1.55 mmol) with activated manganese dioxide (0.404 g, 4.64 mmol) in 5 mL acetone. The crude product was purified by flash chromatography over silica gel (gradient elution, 5-100% EtOAc in CH₂Cl₂) to give pure product 4-isopropyl-1H-imidazole-5-carbaldehyde as a pale pink solid (0.278 g, 37% yield): ¹H NMR (400 MHz, DMSO-D₆) δ 1.23 (d, J=6.8 Hz, 6 H) 3.41 (br s, 0.35 H) 3.53-3.68 (m, 0.65 H) 7.71 (s, 0.65 H) 7.85 (s, 0.35 H) 9.82 (s, 1 H) 12.66 (br s, 0.65 H) 12.91 (br s, 0.35 H).

Step 5: Following the procedure described above in Example 4, 6-amino-8-bromo-4-(3-chloro-4-fluorophenylamino)quinoline-3-carbonitrile (0.300 g, 0.766 mmol, prepared as described in Example 78) was reacted with 4-isopropyl-1H-imidazole-5-carbaldehyde (106 mg, 0.766 mmol) and NaCNBH₃ (32 mg, 0.51 mmol) in 9 mL THF and 3 mL MeOH. The crude product was purified by preparative HPLC, and lyophilized to give a fluffy bright yellow solid (162 mg, 41% yield): ¹H NMR (400 MHz, DMSO-D₆) δ 1.16 (d, J=6.8 Hz, 6 H) 3.09 (br s, 1 H) 4.13 (s, 2 H) 6.49 (s, 1 H) 7.21 (s, 1 H) 7.24-7.33 (m, 1 H) 7.44 (t, J=9.0 Hz, 1 H) 7.48-7.55 (m, 2 H) 7.83 (s,1 H) 8.39 (s, 1 H) 9.45 (s, 1 H) 11.82 (s, 1 H); HRMS (ESI+) calcd for C₂₃H₂₀BrClFN₆ (MH+) 513.0600, found 513.0594.

### Example 116

### 8-bromo-4-(3-chloro-4-fluorophenylamino)-6-((1-methyl-1H-imidazol-4-yl)methylamino)quinoline-3-carbonitrile

Step 1: Following the procedure described above in Example 112, ethyl 1-methyl-1H-imidazole-4-carboxylate (1.00 g, 7.14 mmol) was reacted with a 1.0M THF solution of lithium aluminum hydride (7.1 mL, 7.1 mmol) in 20 mL THF. Work-up gave product (1-methyl-1H-imidazol-4-yl)methanol of sufficient purity to be used directly in the next step (0.806 g, 101% yield): ¹H NMR (400 MHz, DMSO-D₆) δ 3.60 (s, 3 H) 4.30 (s, 2 H) 4.79 (br s,1 H) 6.92 (s, 1 H) 7.45 (s, 1 H).

Step 2: Following the procedure described above in Example 110, (1-methyl-1H-imidazol-4-yl)methanol (0.806 g, 7.19 mmol) was reacted with activated manganese dioxide (1.87 g, 21.6 mmol) in 15 mL acetone. The crude product 1-methyl-1H-imidazole-4-carbaldehyde was purified by flash chromatography over silica gel (gradient elution, 10-100% EtOAc in CH₂Cl₂) to give pure product as a waxy, yellowish solid (0.234 g, 30% yield): ¹H NMR (400 MHz, DMSO-D₆) δ 3.73 (s, 3 H) 7.81 (s, 1 H) 8.00 (s, 1 H) 9.70 (s, 1 H).

Step 3: Following the procedure described above in Example 4, 6-amino-8-bromo-4-(3-chloro-4-fluorophenylamino)quinoline-3-carbonitrile (0.200 g, 0.511 mmol, prepared as described in Example 78) was reacted with 1-methyl-1H-imidazole-4-carbaldehyde (56 mg, 0.51 mmol) and NaCNBH₃ (22 mg, 0.34 mmol) in 6 mL THF and 2 mL MeOH. The yellow precipitate that appeared was collected by suction filtration, washed with MeOH, and dried under vacuum to give pure product as a bright yellow powder (155 mg, 63% yield): ¹H NMR (400 MHz, DMSO-D₆) δ 3.60 (s, 3 H) 4.21 (d, J=5.3 Hz, 2 H) 6.67 (t, J=5.2 Hz, 1 H) 7.05 (s, 1 H) 7.23 (d, J=2.3 Hz, 1 H) 7.28 (ddd, J=8.7, 4.2, 2.8 Hz, 1 H) 7.45 (t, J=9.0 Hz, 1 H) 7.52 (dd, J=6.7, 2.7 Hz, 1 H) 7.54 (s, 1 H) 7.79 (d, J=2.3 Hz, 1 H) 8.38 (s, 1 H) 9.47 (s, 1 H); HRMS (ESI+) calcd for C₂₁H₁₆BrClFN₆ (MH+) 485.0287, found 485.0278.

### Example 117

### N-(8-bromo-4-(3-chloro-4-fluorophenylamino)-3-cyanoquinolin-6-yl)-2-(1H-imidazol-5-yl)acetamide

Following the procedure described above in Example 103, 6-amino-8-bromo-4-(3-chloro-4-fluorophenylamino)quinoline-3-carbonitrile (0.500 g, 1.28 mmol, prepared as described in Example 76) was reacted with imidazoleacetic acid hydrochloride (0.228 g, 1.40 mmol) overnight, in the presence of BOP reagent (0.619 g, 1.40 mmol) and 4-methylmorpholine (0.31 mL, 0.29 g, 2.8 mmol), in 10 mL DMF. LC-MS analysis showed very little product, so the reaction mixture was heated at 60° C. overnight. No additional product was generated, so additional acid (0.228 g), BOP (0.619 g) and 4-methylmorpholine (0.31 mL) were added, and stirring continued at RT overnight. LC-MS analysis showed very little change, so acid (0.684 g), BOP (1.86 g) and 4-methylmorpholine (0.93 mL) were added again, and stirring continued for 3 days at RT. At this point, there finally seemed to be enough product to isolate. The reaction mixture was poured into 100 mL water, and the dark brown precipitate collected by suction filtration, washed with water, and dried under vacuum. This crude product was then purified twice by preparative HPLC and lyophilized to give a pale yellow powder (76 mg, 12% yield): ¹H NMR (400 MHz, DMSO-D₆) δ 3.64 (s, 2 H) 6.98 (s, 1 H) 7.28 (ddd, J=8.8, 4.2, 2.9 Hz, 1 H) 7.44 (t, J=9.0 Hz, 1 H) 7.52 (dd, J=6.6, 2.8 Hz, 1 H) 7.60 (s, 1 H) 8.38 (d, J=2.0 Hz, 1 H) 8.61-8.70 (m, 2 H) 9.95 (s, 1 H) 10.54 (s, 1 H) 12.21 (s, 1 H); HRMS (ESI+) calcd for C₂₁H₁₄BrClFN₆O (MH+) 499.0080, found 499.0071.

### Example 118

### 8-bromo-4-[(3-chloro-4-fluorophenyl)amino]-6-({[5-(2-fluorophenyl)-1H-1,2,3-triazol-4-yl]methyl}amino)quinoline-3-carbonitrile

In a 15 mL round-bottom flask were added 6-amino-4-(4-bromo-3-fluoro-phenylamino)-quinoline-3-carbonitrile (30 mg, 0.076 mmol), ethanol (1 mL) and 5-(2-fluorophenyl)-1H-1,2,3-triazole-4-carbaldehyde (16 mg, 0.08 mmol). Acetic acid was added to bring the pH of the solution to 4, and the mixture was stirred for 15 minutes. Sodium triacetoxyborohydride (32 mg, 0.153 mmol) was then added and the reaction was stirred at RT overnight. The reaction mixture was stripped to dryness and the residue was purified via preparative HPLC and lyophilized to give the product as a yellow solid (8.6 mg, 20%). 1H NMR (400 MHz, MeOD) δ ppm 4.56 (s, 2 H) 7.08 (d, J=2.53 Hz, 1 H) 7.15-7.29 (m, 4 H) 7.34 (dd, J=6.44, 2.65 Hz, 1 H) 7.40-7.49 (m, 1 H) 7.50-7.61 (m, 2 H) 7.75 (s, 1 H) 8.32 (s, 1 H).

### Example 119

### 8-bromo-4-[(3-chloro-4-fluorophenyl)amino]-6-({[5-(3-fluorophenyl)-1H-1,2,3-triazol-4-yl]methyl}amino)quinoline-3-carbonitrile

In a 15 mL round-bottom flask were added 6-amino-4-(4-bromo-3-fluoro-phenylamino)-quinoline-3-carbonitrile (30 mg, 0.076 mmol), ethanol (1 mL) and 5-(3-fluorophenyl)-1H-1,2,3-triazole-4-carbaldehyde (16 mg, 0.08 mmol). Acetic acid was added to bring the pH of the solution to 4, and the mixture was stirred for 15 minutes. Sodium triacetoxyborohydride (32 mg, 0.153 mmol) was then added and the reaction was stirred at RT overnight. The reaction mixture was stripped to dryness and the residue was purified via preparative HPLC and lyophilized to give the product as a yellow solid (6 mg, 14%). 1H NMR (400 MHz, DMSO-D6) δ ppm 4.57 (s, 2 H) 7.18-7.24 (m, 1 H) 7.26-7.31 (m, 1 H) 7.34 (d, J=2.27 Hz, 1 H) 7.41 (t, J=8.97 Hz, 2 H) 7.47-7.62 (m, 4 H) 7.78 (d, J=2.02 Hz, 1 H) 8.43 (s, 1 H).

### Example 120

### 6-(4-(morpholinosulfonyl)benzylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.137 g, 0.44 mmol) was reacted with 4-(morpholinosulfonyl)benzaldehyde (261.9 mg, 1.03 mmol) and NaCNBH₃ (33 mg, 0.53 mmol) in 8 mL EtOH. The crude product was purified by preparative HPLC, and lyophilized to give the product as a yellow solid (156 mg, 53%): 1H NMR (400 MHz, acetonitrile-D3) δ ppm 2.84-2.91 (m, 4 H) 3.61-3.66 (m, 4 H) 4.51 (d, J=6.32 Hz, 2 H) 5.60-5.66 (m, 1 H) 6.76 (d, J=2.53 Hz, 1 H) 7.07-7.13 (m, 1 H) 7.22 (t, J=8.97 Hz, 1 H) 7.28 (dd, J=6.57, 2.53 Hz, 1 H) 7.32 (dd, J=9.09, 2.78 Hz, 1 H) 7.56 (d, J=8.59 Hz, 2 H) 7.60-7.64 (m, 1 H) 7.66-7.71 (m, 2 H) 7.78 (d, J=9.09 Hz, 1 H) 8.38 (s, 1 H); HRMS (ESI+) calcd for C₂₇H₂₃ClFN₅O₃S (MH+) 552.12669, found 552.1262.

### Example 121

### 4-((4-(3-chloro-4-fluorophenylamino)-3-cyanoquinolin-6-ylamino)methyl)benzenesulfonamide

Following the procedure described above in Example 4, 6-amino-4-(3-chloro-4-fluorophenylamino)quinoline-3-carbonitrile (0.3 g, 0.96 mmol) was reacted with 4-formylbenzenesulfonamide (178 mg, 0.96 mmol) and NaCNBH₃ (33 mg, 1.46 mmol) in 7 mL EtOH. The crude product was purified by preparative HPLC, and lyophilized to give the product as a yellow solid (33 mg, 7%): 1H NMR (400 MHz, DMSO-D6) δ ppm 4.48 (d, J=5.81 Hz, 2 H) 6.97 (t, J=5.94 Hz, 1 H) 7.15 (d, J=2.27 Hz, 1 H) 7.19-7.26 (m, 1 H) 7.30 (s, 2 H) 7.35 (dd, J=8.97, 2.40 Hz, 1 H) 7.41 (t, J=8.97 Hz, 1 H) 7.46 (dd, J=6.57, 2.53 Hz, 1 H) 7.55 (d, J=8.59 Hz, 2 H) 7.71 (d, J=9.09 Hz, 1 H) 7.76-7.80 (m, 2 H) 8.32 (s, 1 H) 9.31 (s, 1 H); HRMS (ESI+) calcd for C₂₃H₁₇ClFN₅O₂S (MH+) 482.08483, found 482.0845.

### Example 122

### 6-(4-(4-methylpiperazin-1-ylsulfonyl)benzylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.3 g, 0.96 mmol) was reacted with 4-(4-methylpiperazin-1-ylsulfonyl)benzaldehyde (407 mg, 1.52 mmol) and NaCNBH₃ (72 mg, 1.15 mmol) in 15 mL EtOH. The crude product was purified by preparative HPLC, and lyophilized to give the product as a yellow solid (103 mg, 19%): 1H NMR (400 MHz, DMSO-D6) δ ppm 2.11 (s, 3 H) 2.33 (t, J=4.93 Hz, 4 H) 2.82-2.88 (m, 4 H) 4.52 (d, J=5.81 Hz, 2 H) 7.00 (t, J=5.94 Hz, 1 H) 7.16 (d, J=2.27 Hz, 1 H) 7.18-7.24 (m, 1 H) 7.35 (dd, J=9.47, 1.89 Hz, 1 H) 7.38-7.45 (m, 2 H) 7.61-7.65 (m, 2 H) 7.68-7.74 (m, 3 H) 8.15 (s, 1 H) 8.32 (s, 1 H) 9.30 (s, 1 H); HRMS (ESI+) calcd for C₂₈H₂₆ClFN₆O₂S (MH+) 565.15832, found 565.1581.

### Example 123

### 4-((4-(3-chloro-4-fluorophenylamino)-3-cyanoquinolin-6-ylamino)methyl)-N-(2-(dimethylamino)ethyl)benzenesulfonamide

Following the procedure described above in Example 4, 6-amino-4-(3-chloro-4-fluorophenylamino)quinoline-3-carbonitrile (0.253 g, 0.81 mmol) was reacted with N-(2-(dimethylamino)ethyl)-4-formylbenzenesulfonamide (208 mg, 0.81 mmol) and NaCNBH₃ (73 mg, 1.15 mmol) in 15 mL EtOH. The crude product was purified by combiflash (10%Methanol in dichloromethane), and lyophilized to give the product as a solid (207 mg, 46%): 1H NMR (400 MHz, MeOD) δ ppm 2.15 (s, 6 H) 2.38 (t, J=6.82 Hz, 2 H) 2.89-2.94 (m, 2 H) 4.52 (s, 2 H) 6.99 (d, J=2.53 Hz, 1 H) 7.11-7.16 (m, 1 H) 7.24 (t, J=8.84 Hz, 1 H) 7.30-7.36 (m, 2 H) 7.54 (d, J=8.59 Hz, 2 H) 7.71 (d, J=9.09 Hz, 1 H) 7.78-7.82 (m, J=8.53, 2.15,1.96 Hz, 2 H) 8.27 (s, 1 H); HRMS (ESI+) calcd for C₁₄H₁₆Br₂O₇S (MNa+) 508.88756, found 508.8881.

### Example 124

### 4-(3-bromophenylamino)-8-((dimethylamino)methyl)-6-(pyridin-3-ylmethylamino)quinoline-3-carbonitrile

Following the procedure described above in Example 4, 6-amino-4-(3-bromophenylamino)-8-((dimethylamino)methyl)quinoline-3-carbonitrile (0.19 g, 0.25 mmol) was reacted with nicotinaldehyde (0.06 mL, 0.64 mmol) and NaCNBH₃ (21 mg, 0.33 mmol) in 8 mL EtOH. The crude product was purified by preparative HPLC, and lyophilized to give the product as a solid (92 mg, 75%): 1H NMR (400 MHz, DMSO-D6) δ ppm 2.25 (s, 6 H) 3.95 (s, 2 H) 4.39 (d, J=5.81 Hz, 2 H) 6.97 (t, J=6.06 Hz, 1 H) 7.02 (d, J=2.53 Hz, 1 H) 7.08-7.12 (m, 1 H) 7.24-7.29 (m, 2 H) 7.29-7.31 (m, 1 H) 7.34 (ddd, J=7.83, 4.80, 0.76 Hz, 1 H) 7.49 (d, J=2.53 Hz, 1 H) 7.73 (dt, J=7.83, 2.02 Hz, 1 H) 8.18 (s, 1 H) 8.45 (dd, J=4.80,1.77 Hz, 1 H) 8.58 (d, J=2.27 Hz, 1 H) 9.27 (s, 1 H); HRMS (ESI+) calcd for C₂₅H₂₃BrN₆ (MH+) 487.12403, found 487.1238.

### Example 125

### 6-amino-4-(3-bromophenylamino)-8-methylquinoline-3-carbonitrile

To a 50 mL round-bottomed flask was added 4-(3-bromophenylamino)-8-methyl-6-nitroquinoline-3-carbonitrile (229 mg, 0.6 mmol), SnCl₂.2H₂O (742 mg, 3.28 mmol), and ethyl alcohol (10 mL). The mixture was heated to reflux for 12 hr. After cooling down to RT, water (10 mL) was added followed by sodium bicarbonate (585 mg) and the mixture stirred for 30min. Workup (ethyl acetate/brine) of the reaction gave a solid as product (204 mg, 97%): 1H NMR (400 MHz, DMSO-D6) δ ppm 2.58 (s, 3 H) 5.73 (s, 2 H) 6.95 (d, J=2.53 Hz, 1 H) 6.97-7.02 (m, 1 H) 7.13-7.18 (m, 2 H) 7.19-7.26 (m, 2 H) 8.46 (s, 1 H) 9.25 (s, 1 H); HRMS (ESI+) calcd for C₁₇H₁₃BrN₄ (MH+) 353.03963, found 353.0398.

### Example 126

### 4-(3-bromophenylamino)-8-methyl-6-(pyridin-3-ylmethylamino)quinoline-3-carbonitrile

Following the procedure described above in Example 4, 6-amino-4-(3-bromophenylamino)-8-methylquinoline-3-carbonitrile (0.1 g, 0.28 mmol) was reacted with nicotinaldehyde (0.067 mL, 0.71 mmol) and NaCNBH₃ (28 mg, 0.45 mmol) in 10 mL EtOH. The crude product was purified by preparative HPLC, and lyophilized to give the product as a solid (102 mg, 81 %): 1H NMR (400 MHz, DMSO-D6) δ ppm 2.57 (s, 3 H) 4.37 (d, J=5.81 Hz, 2 H) 6.85 (t, J=6.06 Hz, 1 H) 6.95 (d, J=2.53 Hz, 1 H) 7.06-7.09 (m, 1 H) 7.23-7.30 (m, 4 H) 7.34 (ddd, J=7.83, 4.80, 1.01 Hz, 1 H) 7.73 (dt, J=7.83, 1.89 Hz, 1 H) 8.43 (s, 1 H) 8.45 (dd, J=4.80, 1.77 Hz, 1 H) 8.57 (d, J=2.27 Hz, 1 H) 9.23 (s, 1 H); HRMS (ESI+) calcd for C₂₃H₁₈BrN₅ (MH+) 444.08183, found 444.0837.

### Example 127

### 4-(3-chloro-4-fluorophenylamino)-6-(1-(pyridin-2-yl)ethylamino)quinoline-3-carbonitrile

Following the procedure described above in Example 4, 6-amino-4-(3-chloro-4-fluorophenylamino)quinoline-3-carbonitrile (0.108 g, 0.35 mmol) was reacted with 1-(pyridin-2-yl)ethanone (0.42 g, 3.47 mmol) and NaCNBH₃ (38 mg, 0.6 mmol) in 5 mL EtOH. The crude product was purified by preparative HPLC, and lyophilized to give the product as a solid (61 mg, 42%): 1H NMR (400 MHz, DMSO-D6) δ ppm 1.50 (d, J=6.57 Hz, 3 H) 4.76-4.82 (m, 1 H) 6.87 (d, J=8.59 Hz, 1 H) 7.00 (d, J=2.53 Hz, 1 H) 7.09-7.14 (m, 1 H) 7.21 (ddd, J=7.45, 4.80, 1.14 Hz, 1 H) 7.32-7.41 (m, 4 H) 7.67-7.72 (m, 2 H) 8.16 (s, 1 H) 8.50 (dq, J=4.89, 0.89 Hz, 1 H) 9.25 (s, 1 H); HRMS (ESI+) calcd for C₂₃H₁₇ClFN₅ (MH+) 418.12293, found 418.124.

### Example 128

### 4-(3-chloro-4-fluorophenylamino)-6-((1,5-dimethyl-1H-imidazol-4-yl)methylamino)quinoline-3-carbonitrile

Following the procedure described above in Example 4, 6-amino-4-(3-chloro-4-fluorophenylamino)quinoline-3-carbonitrile (0.102 g, 0.33 mmol) was reacted with 1,5-dimethyl-1H-imidazole-4-carbaldehyde (0.091 g, 0.73 mmol) and NaCNBH₃ (29 mg, 0.46 mmol) in 8 mL EtOH. The crude product was purified by preparative HPLC, and lyophilized to give the product as a solid (120 mg, 88%): 1H NMR (400 MHz, DMSO-D6) δ ppm 2.16 (s, 3 H) 3.51 (s, 3 H) 4.12 (d, J=4.80 Hz, 2 H) 6.32 (t, J=4.42 Hz, 1 H) 7.17 (d, J=2.02 Hz, 1 H) 7.21-7.27 (m, 1 H) 7.36-7.48 (m, 3 H) 7.49 (s, 1 H) 7.66 (d, J=9.09 Hz, 1 H) 8.31 (s,1 H) 9.33 (s, 1 H); HRMS (ESI+) calcd for C₂₂H₁₈ClFN₆ (MH+) 421.13382, found 421.1343.

### Example 129

### 4-(3-chloro-4-fluorophenylamino)-6-((5-methyl-1-(2-morpholinoethyl)-1H-imidazol-4-yl)methylamino)quinoline-3-carbonitrile

Following the procedure described above in Example 4, 6-amino-4-(3-chloro-4-fluorophenylamino)quinoline-3-carbonitrile (0.107 g, 0.34 mmol) was reacted with 5-methyl-1-(2-morpholinoethyl)-1H-imidazole-4-carbaldehyde (0.165 g, 0.74 mmol) and NaCNBH₃ (30 mg, 0.48 mmol) in 10 mL EtOH. The crude product was purified by preparative HPLC, and lyophilized to give the product as a solid (130 mg, 73%): 1H NMR (400 MHz, DMSO-D6) δ ppm 2.19 (s, 3 H) 2.36-2.43 (m, J=4.55 Hz, 4 H) 2.52-2.56 (m, 2 H) 3.51-3.56 (m, 4 H) 3.96 (t, J=6.57 Hz, 2 H) 4.12 (d, J=4.80 Hz, 2 H) 6.35 (t, J=4.80 Hz, 1 H) 7.16 (d, J=2.53 Hz, 1 H) 7.21-7.27 (m, 1 H) 7.37-7.48 (m, 3 H) 7.57 (s, 1 H) 7.67 (d, J=9.09 Hz, 1 H) 8.32 (s, 1 H) 9.31-9.35 (m, 1 H); HRMS (ESI+) calcd for C₂₇H₂₇ClFN₇O (MH+) 520.20224, found 520.203.

### Example 130

### 4-(3-chloro-4-fluorophenylamino)-6-((4-methyl-1-(2-morpholinoethyl)-1H-imidazol-5-yl)methylamino)quinoline-3-carbonitrile

Following the procedure described above in Example 4, 6-amino-4-(3-chloro-4-fluorophenylamino)quinoline-3-carbonitrile (0.111 g, 0.35 mmol) was reacted with 4-methyl-1-(2-morpholinoethyl)-1H-imidazole-5-carbaldehyde (0.328 g, 1.47 mmol) and NaCNBH₃ (35 mg, 0.56 mmol) in 10 mL EtOH. The crude product was purified by preparative HPLC, and lyophilized to give the product as a solid (88mg, 48%): 1H NMR (400 MHz, DMSO-D6) δ ppm 2.13 (s, 3 H) 2.30-2.35 (m, 4 H) 2.57 (t, J=6.69 Hz, 2 H) 3.46-3.51 (m, 4 H) 3.98 (t, J=6.44 Hz, 2 H) 4.24 (d, J=4.80 Hz, 2 H) 6.49 (t, J=4.55 Hz, 1 H) 7.21-7.27 (m, 2 H) 7.33 (dd, J=8.97, 2.15 Hz, 1 H) 7.41-7.48 (m, 2 H) 7.60 (s, 1 H) 7.70 (d, J=9.09 Hz, 1 H) 8.35 (s, 1 H) 9.35 (s,1 H); HRMS (ESI+) calcd for C₂₇H₂₇ClFN₇O (MH+) 520.20224, found 520.2026.

### Example 131

### 4-(3-bromophenylamino)-8-methyl-6-(2-morpholinoethylamino)quinoline-3-carbonitrile

Following the procedure described above in Example 4, 6-amino-4-(3-bromophenylamino)-8-methylquinoline-3-carbonitrile (0.070 g, 0.20 mmol) was reacted with crude morpholin-4-yl-acetaldehyde (0.3 g, 1.71 mmol, made from 4-(2,2-dimethoxyethyl)morpholine) and NaCNBH₃ (20 mg, 0.32 mmol) in 4 mL EtOH. The crude product was purified by preparative HPLC, and lyophilized to give the product as a solid (49 mg, 53%): 1H NMR (400 MHz, DMSO-D6) δ ppm 2.38-2.42 (m, 4 H) 2.57 (s, 3 H) 3.16-3.23 (m, 2 H) 3.55-3.59 (m, 4 H) 6.11 (t, J=5.31 Hz, 1 H) 6.84 (d, J=2.53 Hz, 1 H) 7.06-7.10 (m, 1 H) 7.20-7.30 (m, 4 H) 8.43 (s, 1 H) 9.24 (s, 1 H); HRMS (ESI+) calcd for C₂₃H₂₄BrN₅O (MH+) 466.12370, found 466.1241.

### Example 132

### 6-((5-chloro-1,3-dimethyl-1H-pyrazol-4-yl)methylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.105 g, 0.34 mmol) was reacted with 5-chloro-1,3-dimethyl-1H-pyrazole-4-carbaldehyde (0.140 g, 0.88 mmol) and NaCNBH₃ (33 mg, 0.53 mmol) in 5 mL EtOH. The crude product was purified by preparative HPLC, and lyophilized to give the product as a solid (96 mg, 63%): 1 H NMR (400 MHz, DMSO-D6) δ ppm 2.16 (s, 3 H) 3.73 (s, 3 H) 4.05 (d, J=4.55 Hz, 2 H) 6.41 (t, J=4.55 Hz, 1 H) 7.18 (d, J=2.27 Hz, 1 H) 7.21-7.27 (m, 1 H) 7.32 (dd, J=9.09, 2.53 Hz, 1 H) 7.40-7.48 (m, 2 H) 7.69 (d, J=8.84 Hz, 1 H) 8.34 (s, 1 H) 9.35 (s, 1 H); HRMS (ESI+) calcd for C₂₂H₁₇Cl₂FN₆ (MH+) 455.09485, found 455.0957.

### Example 133

### 4-(3-chloro-4-fluorophenylamino)-6-((1,4-dimethyl-1H-imidazol-5-yl)methylamino)quinoline-3-carbonitrile

Following the procedure described above in Example 4, 6-amino-4-(3-chloro-4-fluorophenylamino)quinoline-3-carbonitrile (0.162 g, 0.52 mmol) was reacted with 1,4-dimethyl-1H-imidazole-5-carbaldehyde (0.080 g, 0.65 mmol) and NaCNBH₃ (46 mg, 0.73 mmol) in 1 OmL EtOH. The crude product was purified by preparative HPLC, and lyophilized to give the product as a solid (32 mg, 15%): 1 H NMR (400 MHz, DMSO-D6) 8 ppm 2.11 (s, 3 H) 3.56 (s, 3 H) 4.22 (d, J=4.55 Hz, 2 H) 6.46-6.51 (m, 1 H) 7.20-7.27 (m, J=2.27 Hz, 2 H) 7.33 (dd, J=9.09, 2.27 Hz, 1 H) 7.39-7.48 (m, 2 H) 7.51 (s, 1 H) 7.69 (d, J=8.84 Hz, 1 H) 8.34 (s, 1 H) 9.37 (s, 1 H); HRMS (ESI+) calcd for C₂₂H₁₈ClFN₆ (MH+) 421.13382, found 421.1337.

### Example 134

### 4-(3-bromophenylamino)-8-((dimethylamino)methyl)-6-(2-morpholinoethylamino)quinoline-3-carbonitrile

Following the procedure described above in Example 4, 6-amino-4-(3-bromophenylamino)-8-((dimethylamino)methyl)quinoline-3-carbonitrile (0.090 g, 0.23 mmol) was reacted with crude morpholin-4-yl-acetaldehyde (0.31 g, 1.77 mmol, made from 4-(2,2-dimethoxyethyl)morpholine) and NaCNBH₃ (23 mg, 0.37 mmol) in 10 mL EtOH. The crude product was purified by preparative HPLC, and lyophilized to give the product as a solid (44 mg, 38%): 1H NMR (400 MHz, MeOD) δ ppm 2.70 (s, 6 H) 2.75-2.82 (m, 4 H) 2.90 (t, J=6.69 Hz, 1 H) 3.59-3.62 (m, 2 H) 3.95-4.02 (m, 4 H) 4.39 (s, 2 H) 7.24 (d, J=2.53 Hz, 1 H) 7.36-7.41 (m, 1 H) 7.51-7.56 (m, 2 H) 7.60 (dd, J=8.34, 2.02 Hz, 2 H) 8.67-8.69 (m, 1 H) 8.83 (s, 1 H); HRMS (ESI+) calcd for C₂₅H₂₉BrN₆O (MH+) 509.16590, found 509.1658.

### Example 135

### 6-((4-chloro-1-methyl-1H-pyrazol-3-yl)methylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.129 g, 0.41 mmol) was reacted with 4-chloro-1-methyl-1H-pyrazole-3-carbaldehyde (0.086 g, 0.59 mmol) and NaCNBH₃ (56 mg, 0.89 mmol) in 4 mL EtOH. The crude product was purified by preparative HPLC, and lyophilized to give the product as a solid (37 mg, 20%): 1H NMR (400 MHz, DMSO-D6) δ ppm 3.79 (s, 3 H) 4.25 (d, J=5.05 Hz, 2 H) 6.53-6.59 (m, 1 H) 7.19-7.26 (m, 2 H) 7.36-7.48 (m, 3 H) 7.69 (d, J=9.09 Hz, 1 H) 7.93 (s, 1 H) 8.34 (s, 1 H) 9.34 (s, 1 H); HRMS (ESI+) calcd for C₂₁H₁₅Cl₂FN₆ (MH+) 441.07920, found 441.0797.

### Example 136

### 2-(2-((4-(3-chloro-4-fluorophenylamino)-3-cyanoquinolin-6-ylamino)methyl)-1H-imidazol-1-yl)acetic acid

To methyl 2-(2-((4-(3-chloro-4-fluorophenylamino)-3-cyanoquinolin-6-ylamino)methyl)-1H-imidazol-1-yl)acetate (264 mg, 0.57 mmol) in tetrahydrofuran (6 mL) and methanol (8 mL) was added lithium hydroxide (1 N, 4.5 mL). After the reaction was complete by TLC, the crude product was purified by preparative HPLC, and lyophilized to give the product as a solid in quantitative yield: 1H NMR (400 MHz, DMSO-D6) δ ppm 4.63-4.74 (m, J=2.78 Hz, 2 H) 5.06 (s, 2 H) 6.99 (s, H) 7.26-7.33 (m, 2 H) 7.34-7.44 (m, 2 H) 7.46 (s, 1 H) 7.50-7.56 (m, 2 H) 7.72 (d, J=9.09 Hz, 1 H) 8.34 (s, 1 H); HRMS (ESI+) calcd for C₂₂H₁₆ClFN₆O₂ (MH+) 451.10800, found 451.1086.

### Example 137

### Methyl 2-(2-((4-(3-chloro-4-fluorophenylamino)-3-cyanoquinolin-6-ylamino)methyl)-1H-imidazol-1-yl)acetate

Following the procedure described above in Example 4, 6-amino-4-(3-chloro-4-fluorophenylamino)quinoline-3-carbonitrile (0.86 g, 2.75 mmol) was reacted with methyl 2-(2-formyl-1H-imidazol-1-yl)acetate (0.659 g, 3.92 mmol) and NaCNBH₃ (230 mg, 3.66 mmol) in lOmL EtOH. The crude product was purified by preparative HPLC, and lyophilized to give the product as a solid (544 mg, 45%): 1H NMR (400 MHz, DMSO-D6) δ ppm 3.60 (s, 3 H) 4.36 (d, J=5.56 Hz, 2 H) 4.97 (s, 2 H) 6.60 (t, J=5.31 Hz, 1 H) 6.85 (d, J=1.26 Hz, 1 H) 7.15 (d, J=1.26 Hz, 1 H) 7.20-7.27 (m, 2 H) 7.33-7.50 (m, 3 H) 7.71 (d, J=9.35 Hz, 1 H) 8.34 (s, 1 H) 9.31 (s, 1 H); HRMS (ESI+) calcd for C₂₃H₁₈ClFN₆O₂ (MH+) 465.12365, found 465.1253.

### Example 138

### 4-(3-chloro-4-fluorophenylamino)-6-((1-(2-morpholino-2-oxoethyl)-1H-imidazol-2-yl)methylamino)quinoline-3-carbonitrile

To a 50 mL round-bottomed flask under nitrogen was added 2-(2-((4-(3-chloro-4-fluorophenylamino)-3-cyanoquinolin-6-ylamino)methyl)-1H-imidazol-1-yl)acetic acid (74 mg, 0.16 mmol), morpholine (0.027 mL, 0.31 mmol), benzotriazol-1-yloxy-tris(dimethylamino)-phosphonium hexafluorophosphate (77 mg, 0.17 mmol), diisopropylethyl amine (0.06 mL, 0.34 mmol) and N,N-dimethylformamide (3 mL). After 12 hr of reaction at RT, the crude product was purified by preparative HPLC, and lyophilized to give the product as a solid (16 mg, 20%): 1H NMR (400 MHz, DMSO-D6) δ ppm 3.34-3.39 (m, 4 H) 3.49 (q, J=5.22 Hz, 4 H) 4.33 (d, J=5.56 Hz, 2 H) 5.02 (s, 2 H) 6.57-6.62 (m, 1 H) 6.83 (s, 1 H) 7.06 (s, 1 H) 7.20-7.26 (m, 2 H) 7.36 (dd, J=9.10, 2.27 Hz, 1 H) 7.40-7.48 (m, 2 H) 7.70 (d, J=9.09 Hz, 1 H) 8.33 (s, 1 H) 9.29 (s, 1 H); HRMS (ESI+) calcd for C₂₆H₂₃ClFN₇O₂ (MH+) 520.16585, found 520.1651.

### Example 139

### 2-(2-((4-(3-chloro-4-fluorophenylamino)-3-cyanoquinolin-6-ylamino)methyl)-1H-imidazol-1-yl)acetamide

Following the procedure described above in Example 138, the desired product was obtained in 31% yield (22 mg from 70 mg of starting acid): 1H NMR (400 MHz, DMSO-D6) δ ppm 4.32 (d, J=5.05 Hz, 2 H) 4.69 (s, 2 H) 6.62 (t, J=4.80 Hz, 1 H) 6.82 (d, J=1.26 Hz, 1 H) 7.12 (d, J-1.01 Hz, 1 H) 7.21 (d, J=2.53 Hz, 1 H) 7.23-7.29 (m, 1 H) 7.30 (s, 1 H) 7.37 (dd, J=8.97, 2.40 Hz, 1 H) 7.43 (t, J=8.97 Hz, 1 H) 7.49 (dd, J=6.57, 2.53 Hz, 1 H) 7.62 (s, 1 H) 7.70 (d, J=8.84 Hz, 1 H) 8.33 (s, 1 H) 9.36 (s, 1 H); HRMS (ESI+) calcd for C₂₂H₁₇ClFN₇0 (MH+) 450.12399, found 450.1233.

### Example 140

### 2-(2-((4-(3-chloro-4-fluorophenylamino)-3-cyanoquinolin-6-ylamino)methyl)-1H-imidazol-1-yl)-N, N-dimethylacetamide

Following the procedure described above in Example 138, the desired product was obtained in 23% yield (17 mg from 70 mg of starting acid): 1H NMR (400 MHz, DMSO-D6) δ ppm 2.77 (s, 3 H) 2.93 (s, 3 H) 4.32 (d, J=5.05 Hz, 2 H) 5.00 (s, 2 H) 6.61 (t, J=4.93 Hz, 1 H) 6.82 (s, 1 H) 7.05 (s, 1 H) 7.18 (d, J=2.02 Hz, 1 H) 7.21-7.28 (m, 1 H) 7.36 (dd, J=8.97, 2.40 Hz, 1 H) 7.42 (t, J=9.09 Hz, 1 H) 7.47 (dd, J=6.44, 2.40 Hz, 1 H) 7.70 (d, J=9.09 Hz, 1 H) 8.32 (s, 1 H) 9.29 (s, 1 H); HRMS (ESI+) calcd for C₂₄H₂₁ClFN₇O (MH+) 478.15529, found 478.1546.

### Example 141

### 4-(3-chloro-4-fluorophenylamino)-6-((1-(2-(4-methylpiperazin-1-yl)-2-oxoethyl)-1H-imidazol-2-yl)methylamino)quinoline-3-carbonitrile

Following the procedure described above in Example 138, the desired product was obtained in 29% yield (24 mg from 70 mg of starting acid): 1H NMR (400 MHz, DMSO-D6) δ ppm 2.03 (s, 3 H) 2.10-2.15 (m, 4 H) 3.28-3.36 (m, 4 H) 4.31 (d, J=5.05 Hz, 2 H) 5.01 (s, 2 H) 6.58 (t, J=4.93 Hz, 1 H) 6.80 (d, J=1.26 Hz, 1 H) 7.05 (d, J=1.26 Hz, 1 H) 7.20-7.26 (m, 2 H) 7.35 (dd, J=9.09, 2.27 Hz, 1 H) 7.41-7.47 (m, 2 H) 7.69 (d, J=9.09 Hz, 1 H) 8.32 (s, 1 H) 9.33 (s, 1 H); HRMS (ESI+) calcd for C₂₇H₂₆ClFN₈O (MH+) 533.19749, found 533.1962.

### Example 142

### tert-butyl 4-(2-(2-((4-(3-chloro-4-fluorophenylamino)-3-cyanoquinolin-6-ylamino)methyl)-1H-imidazol-1-yl)acetamido)piperidine-1-carboxylate

Following the procedure described above in Example 4, the desired product was obtained in 51 % yield (50 mg from 70 mg of starting acid): 1H NMR (400 MHz, DMSO-D6) δ ppm 1.12-1.23 (m, 2 H) 1.37 (s, 9 H) 1.60-1.66 (m, 2 H) 2.71-2.81 (m, 2 H) 3.62-3.77 (m, 3 H) 4.33 (d, J=5.05 Hz, 2 H) 4.69 (s, 2 H) 6.64 (t, J=5.05 Hz, 1 H) 6.81 (d, J=1.26 Hz, 1 H) 7.11 (d, J=1.26 Hz, 1 H) 7.18 (d, J=2.27 Hz, 1 H) 7.22-7.28 (m, 1 H) 7.34-7.38 (m, 1 H) 7.41-7.49 (m, 2 H) 7.70 (d, J=8.84 Hz, 1 H) 8.21 (d, J=7.58 Hz, 1 H) 8.32 (s, 1 H) 9.30 (s, 1 H).

### Example 143

### 2-(2-((4-(3-chloro-4-fluorophenylamino)-3-cyanoquinolin-6-ylamino)methyl)-1H-imidazol-1-yl)-N-(piperidin-4-yl)acetamide

To a 50 mL round-bottomed flask was added tert-butyl 4-(2-(2-((4-(3-chloro-4-fluorophenylamino)-3-cyanoquinolin-6-ylamino)methyl)-1H-imidazol-1-yl)acetamido)piperidine-1-carboxylate (10 mg), trifluoroacetic acid (1.5 mL), and dichloroethane (10 mL). After 30 min of reaction, the reaction mixture was stripped to dryness and the residue was purified via preparative HPLC, and lyophilized to give the product as a yellow solid in quantitative yield: 1H NMR (500 MHz, DMSO-D6) δ ppm 1.17-1.37 (m, 4 H) 1.56-1.84 (m, 4 H) 3.55-3.72 (m, 1 H) 4.34 (d, J=4.58 Hz, 2 H) 4.70 (s, 2 H) 6.63-6.71 (m, 1 H) 6.82 (d, J=7.32 Hz, 1 H) 7.09-7.14 (m, 1 H) 7.19 (d, J=7.63 Hz, 1 H) 7.26 (d, J=6.10 Hz, 1 H) 7.37 (d, J=8.85 Hz, 1 H) 7.42-7.50 (m, 2 H) 7.71 (d, J=9.16 Hz, 1 H) 8.18-8.36 (m, 2 H) 9.31 (s, 1 H); HRMS (ESI+) calcd for C₂₇H₂₆ClFN₈O (MH+) 533.19749, found 533.1972.

The compounds shown in Examples 144-152 were made using the following parallel synthesis strategy:

To a mixture of 6-amino-4-(3-chloro-4-fluorophenylamino)quinoline-3-carbonitrile (50 mg, 0.16 mmol), the corresponding aldehyde (0.32 mmol) and N,N-dimethylformamide (2 mL) was added acetic acid (0.05 mL) and MP-BH₄ (150 mg, 3.Ommol/g, 0.45 mmol). After 12hr of reaction, the mixture was filtered and the filtrate was quenched with PS-isocyanate (500 mg, 1.5 mmol/g, 0.75 mmol). The filtrate was then passed through a cartridge of MP-TsOH (200 mg, 4 mmol/g) and washed with tetrahedron (3×) to remove impurities. The crude product was washed out of the cartridge with 2% NH₄OH in methanol. After preparative HPLC and solvent removal, product was obtained as yellowish solid.

### Example 144

### 6-(2-fluorobenzylamino)-4-(3-chloro-4-fluorophenylamino)quinoline-3-carbonitrile

13.1 mg, 20%: 1H NMR (400 MHz, DMSO-D6) δ ppm 4.43 (d, J=5.31 Hz, 2 H) 6.80 (t, J=5.94 Hz, 1 H) 7.12-7.23 (m, 4 H) 7.29-7.35 (m, 1 H) 7.35-7.45 (m, 4 H) 7.72 (d, J=9.09 Hz, 1 H) 8.33 (s, 1 H) 9.31 (s, 1 H).

### Example 145

### 6-(3-fluorobenzylamino)-4-(3-chloro-4-fluorophenylamino)quinoline-3-carbonitrile

15 mg, 22%: 1H NMR (400 MHz, DMSO-D6) δ ppm 4.41 (d, J=6.06 Hz, 2 H) 6.91 (t, J=6.44 Hz, 1 H) 7.03-7.12 (m, 2 H) 7.15-7.23 (m, 3 H) 7.33-7.43 (m, 4 H) 7.71 (d, J=9.09 Hz, 1 H) 8.33 (s, 1 H) 9.28 (s, 1 H).

### Example 146

### 6-(4-fluorobenzylamino)-4-(3-chloro-4-fluorophenylamino)quinoline-3-carbonitrile

7 mg, 10%: 1H NMR (400 MHz, DMSO-D6) δ ppm 4.36 (d, J=5.56 Hz, 2 H) 6.86 (s, 1 H) 7.10-7.21 (m, 4 H) 7.32-7.44 (m, 5 H) 7.70 (d, J=8.34 Hz, 1 H) 8.33 (s, 1 H) 9.29 (s,1 H).

### Example 147

### 6-(3-bromobenzylamino)-4-(3-chloro-4-fluorophenylamino)quinoline-3-carbonitrile

20 mg, 26%: 1H NMR (400 MHz, DMSO-D6) δ ppm 4.40 (d, J=6.06 Hz, 2 H) 6.93 (t, J=5.94 Hz, 1 H) 7.11 (d, J=2.53 Hz, 1 H) 7.16-7.21 (m, 1 H) 7.28 (t, J=7.83 Hz, 1 H) 7.33-7.46 (m, 5 H) 7.58 (t, J=1.52 Hz, 1 H) 7.71 (d, J=9.09 Hz, 1 (s, 1 H) 9.29 (s, 1 H)

### Example 148

### 6-(3-(trifluoromethoxy)benzylamino)-4-(3-chloro-4-fluorophenylamino)quinoline-3-carbonitrile

18 mg, 23%: 1H NMR (400 MHz, DMSO-D6) δ ppm 4.45 (d, J=6.06 Hz, 2 H) 6.97 (t, J=6.19 Hz, 1 H) 7.13 (d, J=2.53 Hz, 1 H) 7.15-7.20 (m, 1 H) 7.21-7.25 (m, 1 H) 7.33-7.43 (m, 5 H) 7.46 (t, J=7.83 Hz, 1 H) 7.71 (d, J=9.09 Hz, 1 H) 8.33 (s, 1 H) 9.28 (s, 1 H).

### Example 149

### 6-(3-(trifluoromethyl)benzylamino)-4-(3-chloro-4-fluorophenylamino)quinoline-3-carbonitrile

18 mg, 24%: 1H NMR (400 MHz, DMSO-D6) δ ppm 4.49 (d, J=5.81 Hz, 2 H) 6.99 (t, J=6.06 Hz, 1 H) 7.13 (d, J=2.27 Hz, 1 H) 7.15-7.21 (m, 1 H) 7.33-7.42 (m, 3 H) 7.53-7.62 (m, 2 H) 7.65-7.69 (m, 1 H) 7.72 (d, J=8.84 Hz, 1 H) 7.76 (s, 1 H) 8.33 (s, 1 H) 9.28 (s, 1 H).

### Example 150

### 6-(3-phenoxybenzylamino)-4-(3-chloro-4-fluorophenylamino)quinoline-3-carbonitrile

5 mg, 19%: 1H NMR (400 MHz, DMSO-D6) δ ppm 4.38 (d, J=5.81 Hz, 2 H) 6.84-6.91 (m, 2 H) 6.91-6.97 (m, 2 H) 7.02-7.22 (m, 5 H) 7.28-7.49 (m, 6 H) 7.65-7.72 (m, 1 H) 8.32 (s, 1 H) 9.28 (s, 1 H).

### Example 151

### 6-(3-chloro-4-hydroxybenzylamino)-4-(3-chloro-4-fluorophenylamino)quinoline-3-carbonitrile

15 mg, 21 %: 1H NMR (400 MHz, DMSO-D6) δ ppm 4.25 (d, J=6.06 Hz, 2 H) 6.78 (t, J=5.56 Hz, 1 H) 6.91 (d, J=8.34 Hz, 1 H) 7.10-7.16 (m, 2 H) 7.16-7.23 (m, 1 H) 7.31-7.35 (m, 2 H) 7.38-7.46 (m, 2 H) 7.69 (d, J=8.84 Hz, 1 H) 8.32 (s, 1 H) 9.30 (s, 1 H).

### Example 152

### 6-(3-chlorobenzylamino)-4-(3-chloro-4-fluorophenylamino)quinoline-3-carbonitrile

16 mg, 23%: 1H NMR (400 MHz, DMSO-D6) δ ppm 4.40 (d, J=5.81 Hz, 2 H) 6.93 (t, J=6.82 Hz, 1 H) 7.11 (d, J=2.27 Hz, 1 H) 7.15-7.22 (m, 1 H) 7.28-7.45 (m, 7 H) 7.71 (d, J=9.09 Hz, 1 H) 8.33 (s, 1 H) 9.29 (s,1 H).

### Example 153

### 8-bromo-4-[(3-chloro-4-fluorophenyl)amino]-6-[({5-[2-(trifluoromethyl)phenyl]-1H-1,2,3-triazol-4-yl}methyl)amino]quinoline-3-carbonitrile

In a 15 mL round-bottom flask were added 6-amino-4-(4-bromo-3-fluoro-phenylamino)-quinoline-3-carbonitrile (30 mg, 0.076 mmol), ethanol (1 mL) and 5-(2-(trifluoromethyl)phenyl)-1H-1,2,3-triazole-4-carbaldehyde (20 mg, 0.08 mmol). Acetic acid was added to bring the pH of the solution to 4, and the mixture was stirred for 15 minutes. Sodium triacetoxyborohydride (32 mg, 0.153 mmol) was then added and the reaction was stirred at RT overnight. The reaction mixture was stripped to dryness and the residue was purified via preparative HPLC and lyophilized to give the product as a yellow solid (16.8 mg, 34%). 1H NMR (400 MHz, MeOD) δ ppm 4.42 (s, 2 H) 6.99 (d, J=2.27 Hz, 1 H) 7.16-7.30 (m, 2 H) 7.33-7.42 (m, 2 H) 7.45 (d, J=2.27 Hz, 1 H) 7.53-7.62 (m, 2 H) 7.71-7.80 (m, 1 H) 8.25 (s, 1 H).

### Example 154

### 4-(3-chloro-4-fluorophenylamino)-6-((6-((dimethylamino)methyl)-1H-indol-2-yl)methylamino)quinoline-3-carbonitrile

Following the procedure described above in Example 4, 6-amino-4-(3-chloro-4-fluorophenylamino)quinoline-3-carbonitrile (0.100 g, 0.32 mmol) was reacted with 6-((dimethylamino)methyl)-1H-indole-2-carbaldehyde (0.094 g, 0.46 mmol) and NaCNBH₃ (20 mg, 0.32 mmol) in 5 mL EtOH. The crude product was purified by preparative HPLC, and lyophilized to give the product as a solid (103 mg, 65%): 1H NMR (400 MHz, DMSO-D6) δ ppm 2.20 (s, 6 H) 3.53 (s, 2 H) 4.49 (d, J=5.05 Hz, 2 H) 6.34 (s, 1 H) 6.76 (t, J=5.81 Hz, 1 H) 6.91 (dd, J=8.08, 1.26 Hz, 1 H) 7.20-7.28 (m, 3 H) 7.36-7.43 (m, 3 H) 7.43-7.49 (m, 1 H) 7.71 (d, J=9.09 Hz, 1 H) 8.33 (s, 1 H) 9.36 (s, 1 H) 11.10 (d, J=1.52 Hz, 1 H); HRMS (ESI+)calcd for C₂₈H₂₄ClFN₆ (MH+) 499.18077, found 499.1838.

### Example 155

### 2-((4-(3-chloro-4-fluorophenylamino)-3-cyanoquinolin-6-ylamino)methyl)-N,N-dimethyl-1H-indole-6-carboxamide

Following the procedure described above in Example 4, 6-amino-4-(3-chloro-4-fluorophenylamino)quinoline-3-carbonitrile (0.100 g, 0.32 mmol) was reacted with 2-formyl-N,N-dimethyl-1H-indole-6-carboxamide (0.098 g, 0.45 mmol) and NaCNBH₃ (20 mg, 0.32 mmol) in 5 mL EtOH. The crude product was purified by preparative HPLC, and lyophilized to give the product as a solid (36 mg, 22%): 1 H NMR (400 MHz, DMSO-D6) δ ppm 2.96 (s, 6 H) 4.53 (d, J=5.05 Hz, 2 H) 6.41 (d, J=1.26 Hz, 1 H) 6.80 (t, J=5.43 Hz, 1 H) 7.00 (dd, J=8.08, 1.52 Hz, 1 H) 7.21- 7.26 (m, 1 H) 7.27 (d, J=2.53 Hz, 1 H) 7.37-7.49 (m, 5 H) 7.72 (d, J=8.84 Hz, 1 H) 8.33 (s, 1 H) 9.35 (s, 1 H) 11.32 (d, J=1.52 Hz, 1 H); HRMS (ESI+) calcd for C₂₈H₂₂ClFN₆O (MH+) 513.16004, found 513.1618.

### Example 156

### 2-((4-(3-chloro-4-fluorophenylamino)-3-cyanoquinolin-6-ylamino)methyl)-N,N,1-trimethyl-1H-indole-6-carboxamide

Following the procedure described above in Example 4, 6-amino-4-(3-chloro-4-fluorophenylamino)quinoline-3-carbonitrile (0.100 g, 0.32 mmol) was reacted with 2-formyl-N,N,1-trimethyl-1H-indole-6-carboxamide (0.097 g, 0.42 mmol) and NaCNBH₃ (20 mg, 0.32 mmol) in 5 mL EtOH. The crude product was purified by preparative HPLC, and lyophilized to give the product as a solid (128 mg, 76%): 1H NMR (400 MHz, DMSO-D6) δ ppm 2.98 (s, 6 H) 3.78 (s, 3 H) 4.57 (d, J=5.05 Hz, 2 H) 6.50 (s, 1 H) 6.84 (t, J=5.43 Hz, 1 H) 7.04 (dd, J=8.08, 1.52 Hz, 1 H) 7.21-7.29 (m, 2 H) 7.37-7.54 (m, 5 H) 7.72 (d, J=9.09 Hz, 1 H) 8.34 (s, 1 H) 9.34 (s, 1 H); HRMS (ESI+) calcd for C₂₉H₂₄ClFN₆O (MH+) 527.17569, found 527.1762.

### Example 157

### 6-((1H-indol-2-yl)methylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.100 g, 0.32 mmol) was reacted with 1H-indole-2-carbaldehyde (0.100 g, 0.67 mmol) and NaCNBH₃ (20 mg, 0.32 mmol) in 5 mL EtOH. The crude product was purified by preparative HPLC, and lyophilized to give the product as a solid (76 mg, 54%): 1H NMR (400 MHz, DMSO-D6) δ ppm 4.50 (d, J=5.31 Hz, 2 H) 6.36 (d, J=1.01 Hz, 1 H) 6.76 (t, J=5.18 Hz, 1 H) 6.94 (td, J=7.45, 1.01 Hz, 1 H) 7.03 (td, J=7.52,1.14 Hz, 1 H) 7.21-7.28 (m, 2 H) 7.31-7.35 (m, 1 H) 7.38-7.49 (m, 4 H) 8.33 (s, 1 H) 9.36 (s, 1 H) 11.12 (s, 1 H); HRMS (ESI+) calcd for C₂₅H₁₇ClFN₅ (MH+) 442.12293, found 442.1237.

### Example 158

### 6-((1H-imidazol-5-yl)methylamino)-4-(3-chloro-4-fluorophenylamino)-7-isopropoxyquinoline-3-carbonitrile

Following the procedure described above in Example 4, 6-amino-4-(3-chloro-4-fluorophenylamino)-7-isopropoxyquinoline-3-carbonitrile (0.050 g, 0.13 mmol) was reacted with 4(5)-imidazole carboxaldehyde (0.027 g, 0.28 mmol) and NaCNBH₃ (15 mg, 0.24 mmol) in 6 mL EtOH. The crude product was purified by preparative HPLC, and lyophilized to give the product as a solid (55 mg, 91 %): 1H NMR (400 MHz, DMSO-D6) δ ppm 1.38 (d, J=5.81 Hz, 6 H) 3.36-3.41 (m, 1 H) 4.27-4.33 (m, 1 H) 4.88-4.95 (m, 1 H) 5.54 (s, 1 H) 7.03 (s, 1 H) 7.17-7.26 (m, 3 H) 7.38-7.44 (m, 2 H) 7.62 (d, J=11.77 Hz, 1 H) 8.35 (s, 1 H) 9.24 (s, 1 H) 11.96 (s, 1 H); HRMS (ESI+) calcd for C₂₃H₂₀ClFN₆O (MH+) 451.14439; found 451.1456.

### Example 159

### 6-((1H-imidazol-5-yl)methylamino)-4-(3-chloro-4-fluorophenylamino)-7-(3-morpholinopropoxy)quinoline-3-carbonitrile

Following the procedure described above in Example 4, 6-amino-4-(3-chloro-4-fluorophenylamino)-7-(3-morpholinopropoxy)quinoline-3-carbonitrile (0.050 g, 0.11 mmol) was reacted with 4(5)-imidazole carboxaldehyde (0.018 g, 0.19 mmol) and NaCNBH₃ (11 mg, 0.18 mmol) in 6 mL EtOH. The crude product was purified by preparative HPLC, and lyophilized to give the product as a solid (28 mg, 48%): 1H NMR (400 MHz, DMSO-D6) δ ppm 1.94-2.03 (m, 2 H) 2.35-2.41 (m, 4 H) 2.46-2.49 (m, 2 H) 3.55-3.61 (m, 4 H) 4.26 (t, J=6.32 Hz, 2 H) 4.32 (d, J=5.05 Hz, 2 H) 5.64 (t, J=5.81 Hz, 1 H) 7.00 (s, 1 H) 7.17-7.26 (m, 3 H) 7.38-7.43 (m, 2 H) 7.60 (s, 1 H) 8.17 (s, 2 H) 9.25 (s, 1 H); HRMS (ESI+) calcd for C₂₇H₂₇CIFN₇O₂ (MH+) 536.19715, found 536.1973.

### Example 160

### 6-((1H-imidazol-5-yl)methylamino)-4-(3-chloro-4-fluorophenylamino)-7-morpholinoquinoline-3-carbonitrile

Following the procedure described above in Example 4, 6-amino-4-(3-chloro-4-fluorophenylamino)-7-morpholinoquinoline-3-carbonitrile (0.028 g, 0.07 mmol) was reacted with 4(5)-imidazole carboxaldehyde (0.024 g, 0.25 mmol) and NaCNBH₃ (11 mg, 0.18 mmol) in 6 mL EtOH. The crude product was purified by preparative HPLC, and lyophilized to give the product as a solid (12 mg, 36%): 1H NMR (400 MHz, DMSO-D6) δ ppm 2.96-3.03 (m, 4 H) 3.78-3.84 (m, 4 H) 4.33 (d, J=5.56 Hz, 2 H) 5.68 (t, J=5.43 Hz, 1 H) 7.01 (s, 1 H) 7.19-7.25 (m, 1 H) 7.29 (s, 1 H) 7.38-7.47 (m, 3 H) 7.63 (d, J=1.01 Hz, 1 H) 8.20 (s, 1 H) 8.35 (s, 1 H) 9.33 (s, 1 H); HRMS (ESI+) calcd for C₂₄H₂₁ClFN₇O (MH+) 478.15529, found 478.1552.

### Example 161

### 6-((1H-imidazol-5-yl)methylamino)-4-(3-chloro-4-fluorophenylamino)-7-(4-methylpiperazin-1-yl)quinoline-3-carbonitrile

Following the procedure described above in Example 4, 6-amino-4-(3-chloro-4-fluorophenylamino)-7-(4-methylpiperazin-1-yl)quinoline-3-carbonitrile (0.043 g, 0.1Ommol) was reacted with 4(5)-imidazole carboxaldehyde (0.024 g, 0.25 mmol) and NaCNBH₃ (20 mg, 0.32 mmol) in 6 mL EtOH. The crude product was purified by preparative HPLC, and lyophilized to give the product as a solid (33 mg, 64%): 1H NMR (400 MHz, DMSO-D6) δ ppm 2.44 (s, 3 H) 2.80 (s, 4 H) 3.06 (s, 4 H) 4.33 (d, J=5.05 Hz, 2 H) 5.56 (t, J=5.43 Hz, 1 H) 6.51 (s, 1 H) 7.04 (s, 1 H) 7.20-7.26 (m, 1 H) 7.29 (s, 1 H) 7.39-7.47 (m, 3 H) 7.67 (d, J=1.01 Hz, 1 H) 8.35 (s, 1 H) 9.33 (s, 1 H); HRMS (ESI+) calcd for C₂₅H₂₄ClFN₈ (MH+) 491.18692, found 491.1867.

### Example 162

### 6-((1H-imidazol-5-yl)methylamino)-4-(3-chloro-4-fluorophenylamino)-7-(trifluoromethoxy)quinoline-3-carbonitrile

Following the procedure described above in Example 4, 6-amino-4-(3-chloro-4-fluorophenylamino)-7-(trifluoromethoxy)quinoline-3-carbonitrile (0.049 g, 0.12 mmol) was reacted with 4(5)-imidazole carboxaldehyde (0.018 g, 0.19 mmol) and NaCNBH₃ (11 mg, 0.18 mmol) in 6 mL EtOH. The crude product was purified by preparative HPLC, and lyophilized to give the product as a solid (19 mg, 32%): 1 H NMR (400 MHz, DMSO-D6) δ ppm 4.40 (d, J=6.06 Hz, 2 H) 6.35-6.41 (m, 1 H) 6.96 (s, 1 H) 7.27-7.33 (m, 1 H) 7.45 (t, J=8.97 Hz, 1 H) 7.52 (s, 1 H) 7.55 (dd, J=6.95, 2.91 Hz, 1 H) 7.59 (s, 1 H) 7.69 (d, J=1.77 Hz, 1 H) 8.16 (s, 1 H) 8.37 (s, 1 H) 9.54 (s, 1 H); HRMS (ESI+) calcd for C₂₁H₁₃ClF₄N₆O (MH+) 477.08482, found 477.0845.

### Example 163

### 6-((1H-imidazol-5-yl)methylamino)-4-(3-chloro-4-fluorophenylamino)-7-(2-(dimethylamino)ethylthio)quinoline-3-carbonitrile

Following the procedure described above in Example 4, 6-amino-4-(3-chloro-4-fluorophenylamino)-7-(2-(dimethylamino)ethylthio)quinoline-3-carbonitrile (0.032 g, 0.08 mmol) was reacted with 4(5)-imidazole carboxaldehyde (0.020 g, 0.21 mmol) and NaCNBH₃ (15 mg, 0.24 mmol) in 6 mL EtOH. The crude product was purified by preparative HPLC, and lyophilized to give the product as a solid (25 mg, 66%): 1H NMR (400 MHz, DMSO-D6) 6 ppm 2.20 (s, 6 H) 2.54-2.59 (m, 2 H) 3.19 (t, J=6.95 Hz, 2 H) 4.35 (d, J=3.03 Hz, 2 H) 5.68-5.74 (m, 1 H) 6.52 (s, 1 H) 7.04 (s, 1 H) 7.25-7.31 (m, 1 H) 7.33 (s,1 H) 7.44 (t, J=8.97 Hz, 1 H) 7.49-7.53 (m, 1 H) 7.83 (s, 1 H) 8.14 (s, 2 H) 8.35 (s, 1 H) 9.46 (s, 1 H); HRMS (ESI+) calcd for C₂₄H₂₃ClFN₇S (MH+) 496.14809, found 496.1492.

### Example 164

### 6-((1H-imidazol-5-yl)methylamino)-4-(3-chloro-4-fluorophenylamino)-7-(4-(2-(dimethylamino)ethyl)piperazin-1-yl)quinoline-3-carbonitrile

Following the procedure described above in Example 4, 6-amino-4-(3-chloro-4-fluorophenylamino)-7-(4-(2-(dimethylamino)ethyl)piperazin-1-yl)quinoline-3-carbonitrile (0.038 g, 0.08 mmol) was reacted with 4(5)-imidazole carboxaldehyde (0.020 g, 0.21 mmol) and NaCNBH₃ (20 mg, 0.32 mmol) in 6 mL EtOH. The crude product was purified by preparative HPLC, and lyophilized to give the product as a solid (25 mg, 56%): 1H NMR (400 MHz, DMSO-D6) 6 ppm 2.31 (s, 6 H) 2.55-2.70 (m, 8 H) 3.00 (s, 4 H) 4.31 (d, J=5.81 Hz, 2 H) 5.50 (t, J=5.68 Hz, 1 H) 7.03 (s, 1 H) 7.19-7.25 (m, 1 H) 7.28 (s, 1 H) 7.38 (s, 1 H) 7.39-7.46 (m, 2 H) 7.65 (d, J=1.26 Hz, 1 H) 8.16 (s, 3 H) 8.34 (s, 1 H) 9.33 (d, J=1.01 Hz, 1 H); HRMS (ESI+) calcd for C₂₈H₃₁ClFN₉ (MH+) 548.24477, found 548.2456.

### Example 165

### 6-((1H-imidazol-5-yl)methylamino)-4-(3-chloro-4-fluorophenylamino)-7-ethoxyquinoline-3-carbonitrile

Following the procedure described above in Example 4, 6-amino-4-(3-chloro-4-fluorophenylamino)-7-ethoxyquinoline-3-carbonitrile (0.050 g, 0.14 mmol) was reacted with 4(5)-imidazole carboxaldehyde (0.023 g, 0.24 mmol) and NaCNBH₃ (11 mg, 0.18 mmol) in 6 mL EtOH. The crude product was purified by preparative HPLC, and lyophilized to give the product as a solid (19 mg, 31 %): 1H NMR (400 MHz, DMSO-D6) δ ppm 1.44 (t, J=6.95 Hz, 3 H) 4.28 (t, 4 H) 5.59 (t, J=5.31 Hz, 1 H) 7.01 (s, 1 H) 7.16-7.26 (m, 3 H) 7.37-7.44 (m, 2 H) 7.61 (d, J=1.26 Hz, 1 H) 8.15 (s, 1 H) 8.35 (s, 1 H) 9.25 (s, 1 H); HRMS (ESI+) calcd for C₂₂H₁₈ClFN₆O (MH+) 437.12874, found 437.1295.

### Example 166

### 6-((1H-imidazol-5-yl)methylamino)-7-(2-bromoethoxy)-4-(3-chloro-4-fluorophenylamino)quinoline-3-carbonitrile

Following the procedure described above in Example 4, 6-amino-7-(2-bromoethoxy)-4-(3-chloro-4-fluorophenylamino)quinoline-3-carbonitrile (0.049 g, 0.11 mmol) was reacted with 4(5)-imidazole carboxaldehyde (0.025 g, 0.26 mmol) and NaCNBH₃ (16 mg, 0.25 mmol) in 6 mL EtOH. The crude product was purified by preparative HPLC, and lyophilized to give the product as a solid (11 mg, 19%): 1H NMR (400 MHz, DMSO-D6) δ ppm 3.90-3.95 (m, 2 H) 4.34 (d, J=4.55 Hz, 2 H) 4.54-4.59 (m, 2 H) 5.57 (t, J=5.56 Hz, 1 H) 7.05 (s, 1 H) 7.22 (ddd, J=8.84, 4.04, 2.78 Hz, 1 H) 7.29 (d, J=6.32 Hz, 2 H) 7.38-7.45 (m, 2 H) 7.68 (s, 1 H) 8.13 (s, 1 H) 8.36 (s, 1 H) 9.29 (s, 1 H); HRMS (ESI+) calcd for C₂₂H₁₇BrClFN₆O (MH+) 515.03925, found 515.0405.

### Example 167

### 6-(3-(methylsulfonyl)benzylamino)-4-(3-chloro-4-fluorophenylamino)quinoline-3-carbonitrile

Following the procedure described above in Example 4, 6-amino-4-(3-chloro-4-fluorophenylamino)quinoline-3-carbonitrile (0.164 g, 0.52 mmol) was reacted with 3-(methylsulfonyl)benzaldehyde (0.110 g, 0.60 mmol) and NaCNBH₃ (38 mg, 0.60 mmol) in 5 mL EtOH. The crude product was purified by preparative HPLC, and lyophilized to give the product as a solid (136 mg, 54%): 1H NMR (400 MHz, DMSO-D6) δ ppm 3.14 (s, 3 H) 4.52 (d, J=6.06 Hz, 2 H) 7.03 (t, J=6.19 Hz, 1 H) 7.15 (d, J=1.77 Hz, 1 H) 7.18-7.24 (m, 1 H) 7.34-7.46 (m, 3 H) 7.61 (t, J=7.71 Hz, 1 H) 7.69-7.74 (m, 2 H) 7.79-7.83 (m, 1 H) 7.99 (t, J=1.64 Hz, 1 H) 8.32 (s, 1 H) 9.29 (s, 1 H); HRMS (ESI+) calcd for C₂₄H₁₈ClFN₄O₂S (MH+) 481.08958, found 481.0907.

### Example 168

### 3-((4-(3-chloro-4-fluorophenylamino)-3-cyanoquinolin-6-ylamino)methyl)benzenesulfonamide

Following the procedure described above in Example 4, 6-amino-4-(3-chloro-4-fluorophenylamino)quinoline-3-carbonitrile (0.124 g, 0.40 mmol) was reacted with 3-formylbenzenesulfonamide (0.095 g, 0.52 mmol) and NaCNBH₃ (33 mg, 0.53 mmol) in 6 mL EtOH. The crude product was purified by preparative HPLC, and lyophilized to give the product as a solid (61 mg, 32%): 1H NMR (400 MHz, DMSO-D6) δ ppm 4.48 (d, J=6.06 Hz, 2 H) 6.98 (t, J=5.81 Hz, 1 H) 7.17 (d, J=2.53 Hz, 1 H) 7.18-7.24 (m, 1 H) 7.32-7.46 (m, 5 H) 7.52 (t, J=7.83 Hz, 1 H) 7.56-7.60 (m, 1 H) 7.69-7.74 (m, 2 H) 7.86 (t, J=1.52 Hz, 1 H) 8.32 (s, 1 H) 9.30 (s, 1 H); HRMS (ESI+) calcd for C₂₃H₁₇ClFN₅O₂S (MH+) 482.08483, found 482.0855.

### Example 169

### 6-((1H-imidazol-5-yl)methylamino)-4-(3-bromophenylamino)quinoline-3-carbonitrile

Following the procedure described above in Example 4, 6-amino-4-(3-bromophenylamino)quinoline-3-carbonitrile (0.103 g, 0.30 mmol) was reacted with 4(5)-imidazole carboxaldehyde (0.034 g, 0.35 mmol) and NaCNBH₃ (25 mg, 0.40 mmol) in 5 mL EtOH. The crude product was purified by preparative HPLC, and lyophilized to give the product as a solid (88 mg, 69%): 1H NMR (400 MHz, DMSO-D6) δ ppm 4.29 (d, J=4.80 Hz, 1 H) 6.62 (t, J=5.43 Hz, 1 H) 7.13-7.19 (m, 3 H) 7.28-7.32 (m, 2 H) 7.35 (dd, 1 H) 7.38 (dd, J=9.09, 2.27 Hz, 1 H) 7.72 (d, J=9.09 Hz, 1 H) 8.02 (s, 1 H) 8.13 (s, 1 H) 8.39 (s, 1 H) 9.34 (s, 1 H) 12.90 (s, 1 H); HRMS (ESI+) calcd for C₂₀H₁₅BrN₆ (MH+) 419.06143, found 419.0617.

### Example 170

### 8-bromo-4-[(3-chloro-4-fluorophenyl)amino]-6-({[5-(4-fluorophenyl)-1H-1,2,3-triazol-4-yl]methyl}amino)quinoline-3-carbonitrile

In a l5 mL round-bottom flask were added 6-amino-4-(4-bromo-3-fluoro-phenylamino)-quinoline-3-carbonitrile (30 mg, 0.076 mmol), ethanol (1 mL) and 5-(4-fluorophenyl)-1H-1 ,2,3-triazole-4-carbaldehyde (16 mg, 0.08 mmol). Acetic acid was added to bring the pH of the solution to 4, and the mixture was stirred for 15 minutes. Sodium triacetoxyborohydride (32 mg, 0.153 mmol) was then added and the reaction was stirred at RT overnight. The reaction mixture was stripped to dryness and the residue was purified via preparative HPLC and lyophilized to give the product as a yellow solid (3.3 mg, 7.68%). 1H NMR (400 MHz, MeOD) δ ppm 4.58 (s, 2 H) 7.13-7.28 (m, 4 H) 7.39 (d, J=4.55 Hz, 1 H) 7.68-7.74 (m, 2 H) 7.84 (s, 1 H) 8.33 (s, 1 H) 8.56 (s, 1 H).

### Example 171

### 4-(3-chloro-4-fluorophenylamino)-6-((1-methyl-1H-imidazol-2-yl)methylamino)quinoline-3-carbonitrile

Following the procedure described above in Example 4, 6-amino-4-(3-chloro-4-fluorophenylamino)quinoline-3-carbonitrile (0.158 g, 0.51 mmol) was reacted with 1-methyl-1H-imidazole-2-carbaldehyde (0.067 g, 0.70 mmol) and NaCNBH₃ (32 mg, 0.51 mmol) in 6 mL EtOH. The crude product was purified by preparative HPLC, and lyophilized to give the product as a solid (39 mg, 19%): 1H NMR (400 MHz, DMSO-D6) δ ppm 3.64 (s, 3 H) 4.38 (d, J=5.31 Hz, 2 H) 6.66 (t, J=4.42 Hz, 1 H) 6.82 (d, J=1.26 Hz, 1 H) 7.13 (d, J=1.26 Hz, 1 H) 7.22-7.28 (m, 2 H) 7.39-7.50 (m, 3 H) 7.71 (d, J=9.35 Hz, 1 H) 8.34 (s, 1 H) 9.35 (s, 1 H); HRMS (ESI+) calcd for C₂₁H₁₆ClFN₆ (MH+) 407.11818, found 407.1189.

### Example 172

### (R)-4-(3-chloro-4-fluorophenylamino)-6-(1-(pyridin-2-yl)ethylamino)quinoline-3-carbonitrile

4-(3-chloro-4-fluorophenylamino)-6-(1-(pyridin-2-yl)ethylamino)quinoline-3-carbonitrile (prepared as described in Example 127) was subjected to SFC chiral column chromatography to give the desired product: 1H NMR (400 MHz, DMSO-D6) δ ppm 1.50 (d, J=6.57 Hz, 3 H) 4.75-4.84 (m, 1 H) 6.86 (d, J=8.08 Hz, 1 H) 7.00 (d, J=2.53 Hz, 1 H) 7.11 (ddd, J=8.91, 4.23, 2.78 Hz, 1 H) 7.21 (ddd, J=7.58, 4.80, 1.01 Hz, 1 H) 7.31-7.41 (m, 4 H) 7.66-7.72 (m, 2 H) 8.32 (s, 1 H) 8.48-8.51 (m, J=4.83, 1.01, 0.87, 0.87 Hz, 1 H) 9.24 (s, 1 H); HRMS (ESI+) calcd for C₂₃H₁₇ClFN₅ (MH+) 418.12293, found 418.1236.

### Example 173

### (S)-4-(3-chloro-4-fluorophenylamino)-6-(1-(pyridin-2-yl)ethylamino)quinoline-3-carbonitrile

4-(3-chloro-4-fluorophenylamino)-6-(1-(pyridin-2-yl)ethylamino)quinoline-3-carbonitrile (prepared as described in Example 127) was subjected to SFC chiral column chromatography to give the desired product: 1H NMR (400 MHz, DMSO-D6) δ ppm 1.50 (d, J=6.57 Hz, 3 H) 4.75-4.84 (m, 1 H) 6.86 (d, J=8.34 Hz, 1 H) 7.00 (d, J=2.53 Hz, 1 H) 7.11 (ddd, J=8.84, 4.29, 2.78 Hz, 1 H) 7.21 (ddd, J=7.45, 4.80, 1.14 Hz, 1 H) 7.31-7.42 (m, 4 H) 7.66-7.72 (m, 2 H) 8.32 (s, 1 H) 8.47-8.52 (m, 1 H) 9.24 (s, 1 H); HRMS (ESI+) calcd for C₂₃H₁₇ClFN₅ (MH+) 418.12293, found 418.1236.

### Example 174

### Ethyl 2-(4-(3-chloro-4-fluorophenylamino)-3-cyanoquinolin-6-ylamino)acetate

Following the procedure described above in Example 4, 6-amino-4-(3-chloro-4-fluorophenylamino)quinoline-3-carbonitrile (0.313 g, 1.00 mmol) was reacted with ethyl 2-oxoacetate (1 mL, 50% in toluene) and NaCNBH₃ (72 mg, 1.15 mmol) in lOmL EtOH. The crude product was purified by preparative HPLC, and lyophilized to give the product as a solid (334 mg, 84%): 1H NMR (400 MHz, DMSO-D6) δ ppm 1.18 (t, J=7.07 Hz, 3 H) 4.06 (d, J=6.06 Hz, 2 H) 4.13 (q, J=7.07 Hz, 2 H) 6.59-6.69 (m, 1 H) 7.08 (d, J=2.27 Hz, 1 H) 7.21-7.27 (m, 1 H) 7.36-7.51 (m, 3 H) 7.71 (d, J=9.09 Hz, 1 H) 8.33 (s, 1 H) 9.33 (s, 1 H); HRMS (ESI+) calcd for C₂₀H₁₆ClFN₄O₂ (MH+) 399.10186, found 399.1023.

### Example 175

### 2-(4-(3-chloro-4-fluorophenylamino)-3-cyanoquinolin-6-ylamino)acetic acid

Hydrolysis of ethyl 2-(4-(3-chloro-4-fluorophenylamino)-3-cyanoquinolin-6-ylamino)acetate (334 mg, 0.84 mmol) in THF (10 mL) and MeOH (7.5 mL) using lithium hydroxide (1 N, 3 mL) gave the desired product in quantitative yield: 1H NMR (400 MHz, DMSO-D6) δ ppm 3.90 (s, 2 H) 6.42 (s, 1 H) 7.09 (s, 1 H) 7.23-7.31 (m, 1 H) 7.36-7.52 (m, 3 H) 7.65-7.72 (m, 1 H) 8.30 (s, 1 H) 9.36 (s, 1 H); HRMS (ESI+) calcd for C,₈H₁₂ClFN₄O₂ (MH+) 371.07056, found 371.0711.

### Example 176

### 2-(4-(3-chloro-4-fluorophenylamino)-3-cyanoquinolin-6-ylamino)acetamide

2-(4-(3-chloro-4-fluorophenylamino)-3-cyanoquinolin-6-ylamino)acetic acid (9Omg, 0.24 mmol), ammonium chloride (34 mg, 0.64 mmol), benzotriazol-1-yloxy-tris(dimethylamino)-phosphonium hexafluorophosphate (135 mg, 0.31 mmol), diisopropylethyl amine (0.1 4 mL, 0.80 mmol) and N, N-dimethylformamide (12 mL) were mixed together under nitrogen. After 12 hr of reaction at RT, the crude product was purified by preparative HPLC, and lyophilized to give the product as a solid (39 mg, 43%):1H NMR (400 MHz, DMSO-D6) δ ppm 3.78 (d, J=5.81 Hz, 2 H) 6.49 (t, J=5.81 Hz, 1 H) 7.08-7.15 (m, 2 H) 7.20-7.27 (m, 1 H) 7.34-7.48 (m, 3 H) 7.70 (d, J=8.84 Hz, 1 H) 8.25-8.37 (m, 2 H) 9.45 ( s, 1 H); HRMS (ESI+) calcd for C₁₈H₁₃ClFN₅O (MH+) 370.08654, found 370.0853.

### Example 177

### 6-((1H-imidazol-5-yl)methylamino)-8-chloro-4-(3-chloro-4-fluorophenylamino)quinoline-3-carbonitrile

Step 1: A suspension of (Z)-ethyl 3-(2-chloro-4-nitrophenylamino)-2-cyanoacrylate (3.6 g) in Dowtherm (125 mL) under an argon atmosphere was heated to 260° C. for 6hr. After cooling down to RT, hexane (100 mL) was added and precipitate was collected, washed to hexane and dried under vaccum to give solid 8-chloro-4-hydroxy-6-nitroquinoline-3-carbonitrile (2.73 g, 90%): 1H NMR (400 MHz, DMSO-D6) δ ppm 8.73-8.75 (m, 2 H) 8.76-8.78 (m, 1 H) 12.87 (s, 1 H); HRMS (ESI+) calcd for C₁₀H₄ClN₃O₃ (MH+) 250.00140, found 250.0015.

Step 2: A suspension of 8-chloro-4-hydroxy-6-nitroquinoline-3-carbonitrile (2.75 g, 11 .02 mmol) in phosphoryl trichloride (20 mL) was heated to reflux for 12 hr. Then solvent was removed and the residue was poured into a beaker containing ice. Sodium bicarbonate was added until pH=7. The precipitate was filtered, washed with water and dried under vacuum to give solid 4,8-dichloro-6-nitroquinoline-3-carbonitrile (2.75 g, 93%): 1H NMR (400 MHz, DMSO-D6) δ ppm 8.92 (d, J=2.27 Hz, 1 H) 9.01 (d, J=2.27 Hz, 1 H) 9.53 (s, 1 H); HRMS (ESI+) calcd for C₁₀H₃C₁₂N₃O₂ (MH+) 267.96751, found 267.9673.

Step 3: 4,8-dichloro-6-nitroquinoline-3-carbonitrile (645 mg, 2.41 mmol) and 3-chloro-4-fluorobenzenamine (417 mg, 2.88 mmol) were suspended in EtOH (12 mL) under nitrogen atmosphere. The mixture was heated to reflux for 12 hr. The reaction was stripped to dryness and the residue was washed with saturated sodium bicarbonate solution and diethyl ether and dried to give a solid 8-chloro-4-(3-chloro-4-fluorophenylamino)-6-nitroquinoline-3-carbonitrile (605 mg, 67%): 1H NMR (400 MHz, DMSO-D6) δ ppm 7.38-7.46 (m, 1 H) 7.52 (t, J=9.09 Hz, 1 H) 7.68 (d, J=4.04 Hz, 1 H) 8.71 (d, J=2.02 Hz, 1 H) 8.80-8.87 (m, 1 H) 9.51 (d, J=1.52 Hz, 1 H) 10.67 (s, 1 H); HRMS (ESI+) calcd for C₁₆H₇C₁₂FN₄O₂ (MH+) 377.00028, found 377.001.

Step 4: To a 50 mL round-bottomed flask was added 8-chloro-4-(3-chloro-4-fluorophenylamino)-6-nitroquinoline-3-carbonitrile (850 mg, 2.26 mmol), SnCl₂.2H₂O (3100 mg, 13.72 mmol), and ethyl alcohol (30 mL). The mixture was heated to reflux for 3 hr. After cooling down to RT, water (20 mL) was added followed by sodium carbonate to adjust pH to around 7. Workup (ethyl acetate/brine) of the reaction gave solid 6-amino-8-chloro-4-(3-chloro-4-fluorophenylamino)quinoline-3-carbonitrile (636 mg, 81%): 1H NMR (400 MHz, DMSO-D6) δ ppm 5.21 (s, 2 H) 5.93 (s, 1 H) 6.45-6.52 (m, 1 H) 6.65 (dd, J=6.44, 2.65 Hz, 1 H) 7.02 (t, 1 H) 7.14-7.23 (m, 1 H) 7.35-7.47 (m, 2 H); HRMS (ESI+) calcd for C₁₆H₉C₁₂FN₄ (MH+) 347.02610, found 347.0255.

Step 5: Following the procedure described above in Example 4, 6-amino-8-chloro-4-(3-chloro-4-fluorophenylamino)quinoline-3-carbonitrile (0.089 g, 0.26 mmol) was reacted with 4(5)-imidazole carboxaldehyde (0.028 g, 0.29 mmol) and NaCNBH₃ (22 mg, 0.35 mmol) in 6 mL EtOH. The crude product was purified by preparative HPLC, and lyophilized to give the product as a solid (65 mg, 60%): 1H NMR (400 MHz, DMSO-D6) δ ppm 4.26 (d, J=5.05 Hz, 2 H) 6.67 (t, J=5.43 Hz, 1 H) 7.05 (s, 1 H) 7.22 (d, J=2.02 Hz, 1 H) 7.27-7.32 (m, 1 H) 7.45 (t, J=8.97 Hz, 1 H) 7.53 (dd, J=6.57, 2.78 Hz, 1 H) 7.57-7.63 (m, 2 H) 8.16 (s, 1 H) 8.38 (s, 1 H) 9.47 (s, 1 H); HRMS (ESI+) calcd for C₂₀H₁₃C₁₂FN₆ (MH+) 427.06355, found 427.062.

### Example 178

### 8-chloro-4-(3-chloro-4-fluorophenylamino)-6-(pyridin-3-ylmethylamino)quinoline-3-carbonitrile

Following the procedure described above in Example 4, 6-amino-8-chloro-4-(3-chloro-4-fluorophenylamino)quinoline-3-carbonitrile (0.087 g, 0.25 mmol) was reacted with nicotinaldehyde (0.026 mL, 0.28 mmol) and NaCNBH₃ (22 mg, 0.35 mmol) in 9 mL EtOH. The crude product was purified by preparative HPLC, and lyophilized to give the product as a solid (52 mg, 47%): 1H NMR (400 MHz, DMSO-D6) δ ppm 4.44 (d, J=5.81 Hz, 2 H) 6.51 (d, 1 H) 7.01 (t, J=5.94 Hz, 1 H) 7.21 (d, J=2.27 Hz, 1 H) 7.24-7.29 (m, 1 H) 7.37 (dd, J=8.21, 5.18 Hz, 1 H) 7.43 (t, J=9.09 Hz, 1 H) 7.51 (dd, J=6.57, 2.78 Hz, 1 H) 7.55 (d, J=2.27 Hz, 1 H) 7.77-7.80 (m, 1 H) 8.13 (s,1 H) 8.40 (s, 1 H) 8.48 (dd, J=4.67,1.64 Hz, 1 H) 8.62 (d, J=1.77 Hz, 1 H) 9.44 (s, 1 H); HRMS (ESI+) calcd for C₂₂H₁₄Cl₂FN₅ (MH+) 438.06830, found 438.0675.

### Example 179

### 6-(1-(1H-imidazol-5-yl)ethylamino)-4-(3-chloro-4-fluorophenylamino)quinoline-3-carbonitrile

A 25 mL round-bottomed flask under nitrogen atmosphere containing 1-trityl-1H-imidazole-4-carbaldehyde (493 mg, 1.46 mmol) in THF (8 mL) was cooled to −78° C. followed by dropwise addition of methylmagnesium bromide (1.2 mL, 1.4M in THF, 1.68 mmol). The mixture was allowed to warm to RT. The reaction mixture was quenched with water (10 mL) 2 hr later. The white precipitate was collected by filtration and dried to give 1-(1-trityl-1H-imidazol-4-yl)ethanol (439 mg, 85%): 1H NMR (400 MHz, DMSO-D6) δ ppm 1.30 (d, J=6.32 Hz, 3 H) 4.56-4.63 (m, 1 H) 4.86 (d, J=4.80 Hz, 1 H) 6.65-6.67 (m, 1 H) 7.06-7.11 (m, J=6.32, 1.77, 1.52 Hz, 6 H) 7.25 (d, J=1 .52 Hz, 1 H) 7.35-7.45 (m, 9 H); HRMS (ESI+) calcd for 2 C₂₄H₂₂N₂O (MNa+) 731.33564, found 731.337.

To a solution of 1-(1-trityl-1H-imidazol-4-yl)ethanol (300 mg, 0.85 mmol) in dichloromethane under nitrogen atmosphere was added diisopropylethylamine (0.177 mL, 1.02 mmol) followed by methylsulfonyl chloride (0.077 mL, 1 mmol) at 0° C. The mixture was allowed to warm up to RT. After 1 h of reaction, the reaction was worked-up (EtOAc/brine) to give 1-(1-trityl-1H-imidazol-4-yl)ethyl methanesulfonate as a crude solid product which was used for further reaction without purification. To a mixture of 6-amino-4-(3-chloro-4-fluorophenylamino)quinoline-3-carbonitrile (106 mg, 0.34 mmol) and the previously made mesylate (136 mg, 0.31 mmol) was added acetonitrile (15 mL) followed by triethylamine (0.055 mL, 0.39 mmol). The mixture was heated to reflux for 12 hr. The solvent was removed. Reagent grade acetone (100 mL) was added followed by HCl (1 N, 11 mL). The mixture was heated to 60° C. for 2 h. The reaction was stripped to dryness and the residue was purified via preparative HPLC, and lyophilized to give the product as a yellow solid (0.011 g, 5%): 1H NMR (400 MHz, DMSO-D6) δ ppm 1.47 (d, J=6.57 Hz, 3 H) 4.71-4.86 (m, 1 H) 6.39-6.49 (m, J=8.34 Hz, 1 H) 6.90 (s, 1 H) 7.16-7.25 (m, 2 H) 7.33-7.47 (m, 3 H) 7.54 (d, J=1.01 Hz, 1 H) 7.66 (d, J=9.09 Hz, 1 H) 8.28 (s, 2 H) 9.30 (s, 1 H); HRMS (ESI+) calcd for C₂₁H₁₆ClFN₆ (MH+) 407.11818, found 407.1184.

### Example 180

### N-(6-((1H-imidazol-5-yl)methylamino)-3-cyanoquinolin-4-yl)-2-methylpropane-2-sulfonamide

2-Methylpropane-2-sulfonamide (450 mg, 3.28 mmol) and sodium (139 mg, 60% in mineral oil, 3.48 mmol) in DMF (10 mL) in microwave reactor was allowed to stirred at RT for 10 min. Then 4-chloro-6-nitroquinoline-3-carbonitrile (764 mg, 3.27 mmol) in DMF (2 mL) was added and the mixture was heated to 180° C. for 2h. Workup (EtOAc/brine) gave a crude N-(3-cyano-6-nitroquinolin-4-yl)-2-methylpropane-2-sulfonamide. SnCl₂.2H₂O (2.23 g, 9.87 mmol) was added to the crude product in ethanol (15 mL). The mixture was heated to reflux for 2.5h. After cooling down to RT, water (10 mL) was added followed by sodium carbonate to adjust pH to around 7. Workup (ethyl acetate/brine) of the reaction gave crude N-(6-amino-3-cyanoquinolin-4-yl)-2-methylpropane-2-sulfonamide.

Following the procedure described above in Example 4, crude N-(6-amino-3-cyanoquinolin-4-yl)-2-methylpropane-2-sulfonamide was reacted with 4(5)-imidazole carboxaldehyde (0.071 g, 0.74 mmol) and NaCNBH₃ (40 mg, 0.64 mmol) in 10 mL EtOH. The crude product was purified by preparative HPLC, and lyophilized to give the product as a solid (6 mg, 5% overall yield): 1H NMR (400 MHz, DMSO-D6) δ ppm 1.41 (s, 9 H) 4.25 (d, J=1.52 Hz, 2 H) 6.20 (s, 1 H) 7.05 (s, 1 H) 7.19 (dd, J=8.97, 2.65 Hz, 1 H) 7.50 (d, J=8.84 Hz, 1 H) 7.59-7.67 (m, 2 H) 8.18 (s, 1 H) 8.23 (s, 1 H); HRMS (ESI+)calcd for C₁₈H₂₀N₆O₂S (MH+) 385.14412, found 385.1444.

### Example 181

### 6-((1H-imidazol-5-yl)methylamino)-4-(3-chloro-4-fluorophenylamino) 8-hydroxyquinoline-3-carbonitrile

Step 1: 4-chloro-8-methoxy-6-nitroquinoline-3-carbonitrile (400 mg, 1.51 mmol) and 3-chloro-4-fluorobenzenamine (220 mg,. 1.51 mmol) were suspended in EtOH (3.5 mL) in microwave reactor. The mixture was heated to 140° C. for 15 min. The reaction was stripped to dryness and the residue was washed with saturated sodium bicarbonate solution and diethyl ether and dried to give solid 4-(3-chloro-4-fluorophenylamino)-8-methoxy-6-nitroquinoline-3-carbonitrile (491 mg, 87%): 1H NMR (400 MHz, DMSO-D6) δ ppm 4.06-4.20 (m, 3 H) 7.38-7.45 (m, 1 H) 7.52 (t, J=8.97 Hz, 1 H) 7.67 (d, J=5.56 Hz, 1 H) 8.00 (d, J=2.27 Hz, 1 H) 8.74 (s, 1 H) 9.15 (d, J=1.01 Hz, 1 H); HRMS (ESI+) calcd for C₁₇H₁₀ClFN₄O₃ (MH+) 373.04982, found 373.04977.

Step 2: 4-(3-chloro-4-fluorophenylamino)-8-methoxy-6-nitroq uinoline-3-carbonitrile (323 mg, 0.87 mmol) and pyridine hydrochloride (130 mg, 1.12 mmol) in 6 mL of DMF in microwave reactor was heated to 200° C. for 35min. The crude product was purified by preparative HPLC, and lyophilized to give solid 4-(3-chloro-4-fluorophenylamino)-8-hydroxy-6-nitroquinoline-3-carbonitrile (222 mg, 71 %): 1 H NMR (400 MHz, DMSO-D6) δ ppm 7.43 (dd, J=6.82, 2.27 Hz, 1 H) 7.51 (t, J=8.84 Hz, 1 H) 7.69 (d, J=4.80 Hz, 1 H) 7.83 (s, 1 H) 8.73 (s,1 H) 8.98 (s, 1 H) 10.40 (s, 1 H) 10.89 (s,1 H); HRMS (ESI+) calcd for C₁₆H₈ClFN₄O₃ (MH+) 359.03417, found 359.034.

Step 3: 4-(3-chloro-4-fluorophenylamino)-8-hydroxy-6-nitroquinoline-3-carbonitrile (176 mg, 0.49 mmol), SnCl₂.2H₂O (547 mg, 2.42 mmol) in ethyl alcohol (5 mL) in microwave reactor was heated to 110° C. for 1 Omin. After cooling down to RT, water (20 mL) was added followed by sodium carbonate to adjust pH to around 7. Workup (ethyl acetate/brine) of the reaction gave a solid as product (160 mg, 99%): 1H NMR (400 MHz, DMSO-D6) δ ppm 5.64 (s, 2 H) 6.59-6.67 (m, 2 H) 7.09-7.17 (m, 1 H) 7.32-7.43 (m, 2 H) 8.26 (s, 1 H) 9.24 (s, 1 H) 9.56 (s, 1 H); HRMS (ESI+) calcd for C₁₆H₁₀ClFN₄O (MH+) 329.05999, found 329.0601.

Step 4: Following the procedure described above in Example 4, 6-amino-4-(3-chloro-4-fluorophenylamino)-8-hydroxyquinoline-3-carbonitrile (122 mg, 0.37 mmol) was reacted with 4(5)-imidazole carboxaldehyde (0.054 g, 0.56 mmol) and NaCNBH₃ (30 mg, 0.48 mmol) in 10 mL EtOH. The crude product was purified by preparative HPLC, and lyophilized to give the product as a solid (59 mg, 39%): 1H NMR (400 MHz, DMSO-D6) δ ppm 4.16 (d, J=4.80 Hz, 2 H) 6.25-6.33 (m, 1 H) 6.65-6.71 (m, 1 H) 6.94 (s, 1 H) 7.13-7.22 (m, 1 H) 7.33-7.42 (m, 2 H) 7.53 (d, J=1.26 Hz, 1 H) 8.11 (s, 1 H) 8.18 (s,1 H) 9.20 (s, 1 H) 9.42 (s, 1 H); HRMS (ESI+) calcd for C₂₀H₁₄ClFN₆O (MH+) 409.09744, found 409.0975.

### Example 182

### 8-chloro-4-[(3-chloro-4-fluorophenyl)amino]-6-{[(1-oxidopyridin-2-yl)methyl]amino}quinoline-3-carbonitrile

Following the procedure described above in Example 4, 6-amino-8-chloro-4-(3-chloro-4-fluorophenylamino)quinoline-3-carbonitrile (97 mg, 0.28 mmol) was reacted with pyridine-2-carbaldehyde 1-oxide (0.071 g, 0.58 mmol) and NaCNBH₃ (35 mg, 0.56 mmol) in 10 mL EtOH. The crude product was purified by preparative HPLC, and lyophilized to give the product as a solid (64 mg, 50%): 1H NMR (400 MHz, DMSO-D6) δ ppm 4.52 (d, J=6.06 Hz, 2 H) 6.98 (t, J=5.94 Hz, 1 H) 7.08 (d, J=2.02 Hz, 1 H) 7.14-7.37 (m, 5 H) 7.42 (d, J=5.56 Hz, 1 H) 7.55 (s, 1 H) 8.24-8.27 (m, 1 H) 8.30 (s, 1 H) 9.41 (s, 1 H); HRMS (ESI+) calcd for C₂₂H₁₄C₁₂FN₅O (MH+) 454.06322, found 454.0628.

### Example 183

### 8-chloro-4-(3-chloro-4-fluorophenylamino)-6-((1,5-dimethyl-1H-imidazol-4-yl)methylamino)quinoline-3-carbonitrile

Following the procedure described above in Example 4, 6-amino-8-chloro-4-(3-chloro-4-fluorophenylamino)quinoline-3-carbonitrile (79 mg, 0.23 mmol) was reacted with 1 ,5-dimethyl-1H-imidazole-4-carbaldehyde (0.036 g, 0.29 mmol) and NaCNBH₃ (1 8 mg, 0.29 mmol) in 6 mL EtOH. The crude product was purified by preparative HPLC, and lyophilized to give the product as a solid (39 mg, 38%): 1H NMR (400 MHz, DMSO-D6) δ ppm 2.30 (s, 3 H) 3.64 (s, 3 H) 4.26 (d, J=4.80 Hz, 2 H) 6.59-6.68 (m, 1 H) 7.32 (d, J=2.27 Hz, 1 H) 7.39-7.45 (m, 1 H) 7.58 (t, J=8.97 Hz, 1 H) 7.63-7.68 (m, 2 H) 7.74 (d, J=2.27 Hz, 1 H) 8.52 (s, 1 H) 9.59 (s, 1 H); HRMS (ESI+) calcd for C₂₂H₁₇Cl₂FN₆ (MH+) 455.09485, found 455.0946.

### Example 184

### 6-(4-(methylsulfonyl)benzylamino)-8-chloro-4-(3-chloro-4-fluorophenylamino)quinoline-3-carbonitrile

Following the procedure described above in Example 4, 6-amino-8-chloro-4-(3-chloro-4-fluorophenylamino)quinoline-3-carbonitrile (100 mg, 0.29 mmol) was reacted with 4-(methylsulfonyl)benzaldehyde (0.067 g, 0.36 mmol) and NaCNBH₃ (22 mg, 0.35 mmol) in 6 mL EtOH. The crude product was purified by preparative HPLC, and lyophilized to give the product as a solid (38 mg, 26%): 1H NMR (400 MHz, DMSO-D6) δ ppm 3.12 (s, 3 H) 4.47 (d, J=6.06 Hz, 2 H) 7.05 (t, J=5.94 Hz, 1 H) 7.12 (d, J=2.27 Hz, 1 H) 7.16-7.22 (m, 1 H) 7.36 (t, J=9.09 Hz, 1 H) 7.41-7.46 (m, 1 H) 7.49 (d, J=2.02 Hz, 1 H) 7.57 (d, J=8.34 Hz, 1 H) 7.81-7.87 (m, 2 H) 8.32 (s, 1 H) 9.37 (s, 1 H); HRMS (ESI+) calcd for C₂₄H₁₇Cl₂FN₄O₂S (MH+) 515.05060, found 515.0521.

### Example 185

### 6-(3-(methylsulfonyl)benzylamino)-8-chloro-4-(3-chloro-4-fluorophenylamino)quinoline-3-carbonitrile

Following the procedure described above in Example 4, 6-amino-8-chloro-4-(3-chloro-4-fluorophenylamino)quinoline-3-carbonitrile (75 mg, 0.22 mmol) was reacted with 3-(methylsulfonyl)benzaldehyde (0.040 g, 0.22 mmol) and NaCNBH₃ (16 mg, 0.25 mmol) in 1 OmL EtOH. The crude product was purified by preparative HPLC, and lyophilized to give the product as a solid (13 mg, 12%): 1H NMR (400 MHz, DMSO-D6) δ ppm 3.09 (s, 3 H) 4.47 (d, J=5.81 Hz, 2 H) 7.08 (t, J=6.06 Hz, 1 H) 7.12 (d, J=2.27 Hz, 1 H) 7.17-7.22 (m, 1 H) 7.36 (t, J=8.97 Hz, 1 H) 7.44 (dd, J=6.69, 2.65 Hz, 1 H) 7.56 (t, J=7.71 Hz, 1 H) 7.66 (d, J=7.58 Hz, 1 H) 7.75 (dd, J=7.58, 1.77 Hz, 1 H) 7.93 (s, 1 H) 8.15 (s, 1 H) 8.32 (s, 1 H) 9.37 (s, 1 H); HRMS (ESI+) calcd for C₂₄H₁₇Cl₂FN₄O₂S (MH+) 515.05060, found 515.0519.

### Example 186

### 4-((8-chloro-4-(3-chloro-4-fluorophenylamino)-3-cyanoquinolin-6-ylamino)methyl)benzenesulfonamide

Following the procedure described above in Example 4, 6-amino-8-chloro-4-(3-chloro-4-fluorophenylamino)quinoline-3-carbonitrile (114 mg, 0.33 mmol) was reacted with 4-formylbenzenesulfonamide (0.080 g, 0.43 mmol) and NaCNBH₃ (27 mg, 0.43 mmol) in 6 mL EtOH. The crude product was purified by preparative HPLC, and lyophilized to give the product as a solid (95 mg, 56%): 1H NMR (400 MHz, DMSO-D6) δ ppm 4.42 (d, J=5.56 Hz, 2 H) 7.00 (t, 1 H) 7.13 (d, J=2.02 Hz, 1 H) 7.17-7.22 (m, 1 H) 7.24 (s, 2 H) 7.36 (t, J=8.97 Hz, 1 H) 7.42-7.50 (m, 4 H) 7.72 (d, J=8.59 Hz, 2 H) 8.30 (s, 1 H) 9.38 (s, 1 H); HRMS (ESI+) calcd for C₂₃H₁₆Cl₂FN₅O₂S (MH+) 516.04585, found 516.0469.

### Example 187

### 6-((H-imidazo[1,2-a]pyridin-2-yl)methylamino)-8-chloro-4-(3-chloro-4-fluorophenylamino)quinoline-3-carbonitrile

Following the procedure described above in Example 4, 6-amino-8-chloro-4-(3-chloro-4-fluorophenylamino)quinoline-3-carbonitrile (94 mg, 0.27 mmol) was reacted with H-imidazo[1,2-a]pyridine-2-carbaldehyde (0.051 g, 0.35 mmol) and NaCNBH₃ (24 mg, 0.38 mmol) in 4 mL EtOH. The crude product was purified by preparative HPLC, and lyophilized to give the product as a solid (69 mg, 53%): 1H NMR (400 MHz, DMSO-D6) δ ppm 4.75 (d, J=5.31 Hz, 2 H) 6.53 (d, 1 H) 6.98 (t, J=5.18 Hz, 1 H) 7.05 (t, J=6.82 Hz, 1 H) 7.29-7.33 (m, 1 H) 7.36 (d, J=2.27 Hz, 1 H) 7.46 (t, J=9.09 Hz, 1 H) 7.52 (d, J=2.27 Hz, 1 H) 7.55 (dd, J=6.69, 2.65 Hz, 1 H) H); HRMS (ESI+) calcd for C₂₄H₁₅Cl₂FN₆ (MH+) 477.07920, found 477.0794.

### Example 188

### 8-chloro-4-(3-chloro-4-fluorophenylamino)-6-((2,3-dihydropyrazolo[5, 1-b]oxazol-6-yl)methylamino)quinoline-3-carbonitrile

Following the procedure described above in Example 4, 6-amino-8-chloro-4-(3-chloro-4-fluorophenylamino)quinoline-3-carbonitrile (85 mg, 0.24 mmol) was reacted with 2,3-dihydropyrazolo[5,1-b]oxazole-6-carbaldehyde (0.034 g, 0.25 mmol) and NaCNBH₃ (24 mg, 0.38 mmol) in 6 mL EtOH. The crude product was purified by preparative HPLC, and lyophilized to give the product as a solid (36 mg, 31 %): 1H NMR (400 MHz, DMSO-D6) δ ppm 4.14-4.30 (m, 4 H) 4.97-5.09 (m, 2 H) 5.42 (s, 1 H) 6.74 (t, J=5.18 Hz, 1 H) 7.22 (d, J=2.27 Hz, 1 H) 7.24-7.32 (m, 1 H) 7.44 (t, J=8.97 Hz, 1 H) 7.50-7.54 (m, 1 H) 7.57 (d, J=2.02 Hz, 1 H) 8.38 (s, 1 H) 9.48 (s, 1 H); HRMS (ESI+) calcd for C₂₂H₁₅Cl₂FN₆O (MH+) 469.07412, found 469.0737.

### Example 189

### 8-chloro-4-(3-chloro-4-fluorophenylamino)-6-((5,6-dihydro-4H-pyrrolo[1,2-b]pyrazol-2-yl)methylamino)quinoline-3-carbonitrile

Following the procedure described above in Example 4, 6-amino-8-chloro-4-(3-chloro-4-fluorophenylamino)quinoline-3-carbonitrile (80 mg, 0.23 mmol) was reacted with 5,6-dihydro-4H-pyrrolo[1,2-b]pyrazole-2-carbaldehyde (0.041 g, 0.30 mmol) and NaCNBH₃ (24 mg, 0.38 mmol) in 6 mL EtOH. The crude product was purified by preparative HPLC, and lyophilized to give the product as a solid (36 mg, 34%): 1H NMR (400 MHz, DMSO-D6) δ ppm 2.45-2.55 (m, 2 H) 2.73-2.86 (m, 2 H) 3.95-4.07 (m, 2 H) 4.27 (d, J=5.31 Hz, 2 H) 5.97 (s, 1 H) 6.74 (s, 1 H) 7.22 (d, J=2.02 Hz, 1 H) 7.24-7.31 (m, 1 H) 7.45 (t, J=9.09 Hz, 1 H) 7.52 (d, J=6.82 Hz, 1 H) 7.58 (d, J=2.02 Hz, 1 H) 8.38 (s, 1 H) 9.48 (s, 1 H); HRMS (ESI+) calcd for C₂₃H₁₇Cl₂FN₆ (MH+) 467.09485, found 467.0945.

### Example 190

### 8-chloro-4-(3-chloro-4-fluorophenylamino)-6-((2-ethyl-5-methyl-1H-imidazol-4-yl)methylamino)quinoline-3-carbonitrile

Following the procedure described above in Example 4, 6-amino-8-chloro-4-(3-chloro-4-fluorophenylamino)quinoline-3-carbonitrile (80 mg, 0.23 mmol) was reacted with 2-ethyl-5-methyl-1H-imidazole-4-carbaldehyde (0.067 g, 0.49 mmol) and NaCNBH₃ (24 mg, 0.38 mmol) in 6 mL EtOH. The crude product was purified by preparative HPLC, and lyophilized to give the product as a solid (52 mg, 48%): 1H NMR (400 MHz, DMSO-D6) δ ppm 1.17 (t, J=7.58 Hz, 3 H) 2.06-2.17 (m, 3 H) 2.51-2.60 (m, 2 H) 4.09 (d, J=4.55 Hz, 2 H) 6.53 (t, J=4.80 Hz, 1 H) 7.18 (t, J=2.27 Hz, 1 H) 7.25-7.32 (m, 1 H) 7.45 (t, J=8.97 Hz, 1 H) 7.52 (dd, J=6.57, 2.78 Hz, 1 H) 7.60 (d, J=2.27 Hz, 1 H) 8.16 (s, 1 H) 8.39 (s, 1 H) 9.47 (s, 1 H); HRMS (ESI+) calcd for C₂₃H₁₉Cl₂FN₆ (MH+) 469.11050, found 469.1102.

### Example 191

### 2-(4-((8-chloro-4-(3-chloro-4-fluorophenylamino)-3-cyanoquinolin-6-ylamino)methyl)-2-methyl-1H-imidazol-1-yl)acetamide

Following the procedure described above in Example 4, 6-amino-8-chloro-4-(3-chloro-4-fluorophenylamino)quinoline-3-carbonitrile (86 mg, 0.25 mmol) was reacted with 2-(4-formyl-2-methyl-1H-imidazol-1-yl)acetamide (0.043 g, 0.24 mmol) and NaCNBH₃ (24 mg, 0.38 mmol) in 6 mL EtOH. The crude product was purified by preparative HPLC, and lyophilized to give the product as a solid (15 mg, 12%): 1H NMR (400 MHz, DMSO-D6) δ ppm 2.19 (s, 3 H) 4.16 (d, J=5.05 Hz, 2 H) 4.50 (s, 2 H) 6.65 (t, J=5.18 Hz, 1 H) 6.94 (s, 1 H) 7.19 (d, J=2.27 Hz, 1 H) 7.24 (s, 1 H) 7.26-7.32 (m, 1 H) 7.45 (t, J=9.09 Hz, 1 H) 7.50-7.55 (m, 1 H) 7.61 (d, J=2.27 Hz, 1 H) 8.19 (s, 1 H) 8.37 (s, 1 H) 9.47 (s, 1 H); HRMS (ESI+) calcd for C₂₃H₁₈Cl₂FN₇O (MH+) 496.08611, found 496.0878.

### Example 192

### 8-chloro-4-(3-chloro-4-fluorophenylamino)-6-((6-methylpyridin-2-yl)methylamino)quinoline-3-carbonitrile

Following the procedure described above in Example 4, 6-amino-8-chloro-4-(3-chloro-4-fluorophenylamino)quinoline-3-carbonitrile (80 mg, 0.23 mmol) was reacted with 6-methylpicolinaldehyde (0.120 g, 0.99 mmol) and NaCNBH₃ (24 mg, 0.38 mmol) in 6 mL EtOH. The crude product was purified by preparative HPLC, and lyophilized to give the product as a solid (62 mg, 59%): 1H NMR (400 MHz, DMSO-D6) δ ppm 2.46 (s, 3 H) 4.49 (d, J=6.06 Hz, 2 H) 7.06 (t, J=5.94 Hz, 1 H) 7.14 (dd, J=9.85, 7.83 Hz, 2 H) 7.19 (d, J=2.27 Hz, 1 H) 7.21-7.26 (m, 1 H) 7.41 (t, J=8.97 Hz, 1 H) 7.47 (dd, J=6.57, 2.53 Hz, 1 H) 7.60-7.67 (m, 2 H) 8.39 (s, 1 H) 9.45 (s, 1 H); HRMS (ESI+) calcd for C₂₃H₁₆Cl₂FN₅ (MH+) 452.08395, found 452.0834.

### Example 193

### 2-(4-((8-chloro-4-(3-chloro-4-fluorophenylamino)-3-cyanoquinolin-6-ylamino)methyl)-2-ethyl-1H-imidazol-1-yl)acetamide

Following the procedure described above in Example 4, 6-amino-8-chloro-4-(3-chloro-4-fluorophenylamino)quinoline-3-carbonitrile (86 mg, 0.25 mmol) was reacted with 2-(2-ethyl-4-formyl-1H-imidazol-1-yl)acetamide (0.043 g, 0.24 mmol) and NaCNBH₃ (24 mg, 0.38 mmol) in 6 mL EtOH. The crude product was purified by preparative HPLC, and lyophilized to give the product as a solid (13 mg, 10%): 1H NMR (400 MHz, DMSO-D6) δ ppm 1.17 (t, J=7.45 Hz, 3 H) 2.52-2.56 (m, 2 H) 4.20 (d, J=5.56 Hz, 2 H) 4.50 (s, 2 H) 6.67 (t, J=5.43 Hz, 1 H) 6.93 (s, 1 H) 7.21 (d, J=2.27 Hz, 1 H) 7.24 (s, 1 H) 7.26-7.32 (m, 1 H) 7.45 (t, J=9.09 Hz, 1 H) 7.51-7.55 (m, 2 H) 7.61 (d, J=2.27 Hz, 1 H) 8.37 (s, 1 H) 9.47 (s, 1 H); HRMS (ESI+) calcd for C₂₄H₂₀Cl₂FN₇O (MH+) 512.11632, found 512.115.

### Example 194

### tert-butyl 4-((8-chloro-4-(3-chloro-4-fluorophenylamino)-3-cyanoquinolin-6-ylamino)methyl)-2-ethyl-5-methyl-1H-imidazole-1-carboxylate

Following the procedure described above in Example 4, 6-amino-8-chloro-4-(3-chloro-4-fluorophenylamino)quinoline-3-carbonitrile (98 mg, 0.28 mmol) was reacted with tert-butyl 2-ethyl-4-formyl-5-methyl-1H-imidazole-1-carboxylate (0.066 g, 0.28 mmol) and NaCNBH₃ (24 mg, 0.38 mmol) in 6 mL EtOH. The crude product was purified by preparative HPLC, and lyophilized to give the product as a solid (88 mg, 55%): 1H NMR (400 MHz, DMSO-D6) δ ppm 1.17 (t, J=7.45 Hz, 3 H) 1.55(s,9 H) 2.30 (s, 3 H) 2.84 (q, J=7.33 Hz, 2 H) 4.13 (d, J=4.80 Hz, 2 H) 6.63 (t, J=5.05 Hz, 1 H) 7.20 (d, J=2.27 Hz, 1 H) 7.24-7.30 (m, 1 H) 7.44 (t, J=8.97 Hz, 1 H) 7.50 (dd, J=6.57, 2.53 Hz, 1 H) 7.58 (d, J=2.27 Hz, 1 H) 8.41 (s, 1 H) 9.46 (s, 1 H); HRMS (ESI+) calcd for C₂₈H₂₇Cl₂FN₆O₂ (MH+) 569.16293, found 569.1617.

### Example 195

### 8-chloro-4-[(3-chloro-4-fluorophenyl)amino]-6-{[(6-methyl-1-oxidopyridin-2- yl)methyl]amino}quinoline-3-carbonitrile

Following the procedure described above in Example 4, 6-amino-8-chloro-4-(3-chloro-4-fluorophenylamino)quinoline-3-carbonitrile (116 mg, 0.33 mmol) was reacted with 6-methylpyridine-2-carbaldehyde 1-oxide (0.152 g, 1.11 mmol) and NaCNBH₃ (31 mg, 0.49 mmol) in 12 mL EtOH. The crude product was purified by preparative HPLC, and lyophilized to give the product as a solid (7 mg, 4%): 1H NMR (400 MHz, DMSO-D6) δ ppm 1.75 (s, 1 H) 2.40 (s, 3 H) 4.61 (s, 2 H) 7.13-7.17 (m, J=2.27 Hz, 1 H) 7.20-7.27 (m, 2 H) 7.37-7.44 (m, 2 H) 7.50 (dd, J=6.57, 2.53 Hz, 1 H) 7.64 (d, J=2.27 Hz, 1 H) 8.39 (s, 1 H) 9.53 (s, 1 H); HRMS (ESI+) calcd for C₂₃H₁₆Cl₂FN₅O (MH+) 468.07887, found 468.0787.

### Example 196

### 8-chloro-4-[(3-chloro-4-fluorophenyl)amino]-6-{[(3-methyl-1-oxidopyridin-2- yl)methyl]amino}quinoline-3-carbonitrile

Following the procedure described above in Example 4, 6-amino-8-chloro-4-(3-chloro-4-fluorophenylamino)quinoline-3-carbonitrile (130 mg, 0.37 mmol) was reacted with 3-methylpyridine-2-carbaldehyde 1-oxide (0.90 g, 0.66 mmol) and NaCNBH₃ (31 mg, 0.49 mmol) in 6 mL EtOH. The crude product was purified by preparative HPLC, and lyophilized to give the product as a solid (29 mg, 17%): 1H NMR (400 MHz, DMSO-D6) δ ppm 2.40 (s, 3 H) 4.63 (d, J=5.05 Hz, 2 H) 6.74 (d, J=5.81 Hz, 1 H) 7.22-7.34 (m, 2 H) 7.41-7.49 (m, 2 H) 7.53-7.57 (m, 1 H) 7.59 (d, J=2.53 Hz, 1 H) 8.22 (d, J=7.33 Hz, 1 H) 8.41 (s, 1 H) 9.45 (s, 1 H); HRMS (ESI+) calcd for C₂₃H₁₆Cl₂FN₅O (MH+) 468.07887, found 468.0785.

### Example 197

### 4-[(3-chloro-4-fluorophenyl)amino]-8-iodo-6-nitroquinoline-3-carbonitrile

4-chloro-8-iodo-6-nitroquinoline-3-carbonitrile (4.64 g, 12.92 mmol) and 3-chloro-4-fluorobenzenamine (2.3 g, 15.80 mmol) were suspended in EtOH (70 mL) under nitrogen atmosphere. The mixture was heated to reflux for 12 hr. The reaction was stripped to dryness and the residue was washed with saturated sodium bicarbonate solution and diethyl ether and dried to give a solid product in quantitative yield: 1H NMR (400 MHz, DMSO-D6) δ ppm 6.71-6.80 (m, 1 H) 6.89 (d, J=6.57 Hz, 1 H) 7.18 (t, J=9.09 Hz, 1 H) 7.98 (s, 1 H) 8.63 (d, J=2.53 Hz, 1 H) 9.14 (d, J=2.53 Hz, 1 H); HRMS (ESI+) calcd for C₁6H₇ClFIN₄O₂ (MH+) 468.93590, found 468.9362.

### Example 198

### 6-({[1-[(benzyloxy)methyl]-4-(3-hydroxypropyl)-1H-imidazol-5-yl]methyl}amino)-8-chloro-4-[(3-chloro-4-fluorophenyl)amino]quinoline-3-carbonitrile

To a mixture of 1-(benzyloxymethyl)-4-iodo-1H-imidazole-5-carbaldehyde (715 mg, 2.09 mmol) and PdCl₂(PPh₃)₂ (85 mg, 0.12 mmol) in DMF (5 mL) under nitrogen atmosphere was added Et₃N (1.1 mL) followed by prop-2-yn-1-ol (0.245 mL, 4.21 mmol). The mixture was heated to 90° C. for 4 hr. The reaction was purified via preparative HPLC to give a liquid product 1-(benzyloxymethyl)-4-(3-hydroxyprop-1-ynyl)-1H-imidazole-5-carbaldehyde (405 mg, 72%).

Following the procedure described above in Example 4, 6-amino-8-chloro-4-(3-chloro-4-fluorophenylamino)quinoline-3-carbonitrile (209 mg, 0.60 mmol) was reacted with 1-(benzyloxymethyl)-4-(3-hydroxyprop-1-ynyl)-1H-imidazole-5-carbaldehyde (0.187 g, 0.69 mmol) and NaCNBH₃ (42 mg, 0.67 mmol) in 10 mL EtOH. The crude product was purified by preparative HPLC, and lyophilized to give 6-((1-(benzyloxymethyl)-4-(3-hydroxyprop-1-ynyl)-1H-imidazol-5-yl)methylamino)-8-chloro-4-(3-chloro-4-fluorophenylamino)quinoline-3-carbonitrile as a solid (80 mg, 22%). Hydrogenation of the solid was carried out according to procedure described in Example 119 to give the desired product as a solid (1 8 mg, 22%):1H NMR (400 MHz, acetonitrile-D3) δ ppm 1.66-1.72 (m, 2 H) 2.55 (t, J=7.20 Hz, 2 H) 3.42 (t, J=5.94 Hz, 2 H) 4.25 (d, J=4.80 Hz, 2 H) 4.38 (s, 2 H) 5.08 (s, 1 H) 5.28 (s, 2 H) 6.87 (d, J=2.02 Hz, 1 H) 7.11-7.21 (m, 8 H) 7.30 (dd, J=6.32, 2.27 Hz, 1 H) 7.51 (s, 1 H) 7.81 (s, 1 H) 8.00 (s, 1 H) 8.35 (s, 1 H); HRMS (ESI+) calcd for C₃₁H₂₇Cl₂FN₆O₂ (MH+) 605.16293, found 605.1645.

### Example 199

### 8-chloro-4-[(3-chloro-4-fluorophenyl)amino]-6-(ethylamino)quinoline-3-carbonitrile

The product was isolated from the synthesis of 6-((1-(benzyloxymethyl)-4-(3-hydroxyprop-1-ynyl)-1H-imidazol-5-yl)methylamino)-8-chloro-4-(3-chloro-4-fluorophenylamino)quinoline-3-carbonitrile in Example 209:1H NMR (400 MHz, DMSO-D6) δ ppm 1.22 (t, J=7.07 Hz, 3 H) 3.10-3.23 (m, 2 H) 6.41-6.49 (m, 1 H) 7.06 (d, J=2.27 Hz, 1 H) 7.22-7.32 (m, 2 H) 7.42-7.47 (m, 2 H) 7.51 (dd, J=6.57, 2.78 Hz, 1 H) 8.37 (s, 1 H) 9.46 (s,1 H); HRMS (ESI+) calcd for C₁₈H₁₃Cl₂FN₄ (MH+) 375.05740, found 375.0574.

### Example 200

### 8-chloro-4-[(3-chloro-4-fluorophenyl)amino]-6-{[2-(1H-tetrazol-5-yl)ethyl]amino}quinoline-3-carbonitrile

A mixture of 3,3-diethoxypropanenitrile (1 mL, 6.66 mmol) and azidotributylstannane (2.38 mL, 8.69 mmol) in ethyleneglycol diethylether (18 mL) under nitrogen atmosphere was heated to reflux for 24 hr. The reaction was stripped to dryness. Hydrochloric acid (˜1.25N in methanol, 50 mL) was added followed by water (0.5 mL). The mixture was heated to reflux for 5 h. The reaction was stripped to dryness and the crude material was used for reaction without purification.

Following the procedure described above in Example 4, 6-amino-8-chloro-4-(3-chloro-4-fluorophenylamino)quinoline-3-carbonitrile (102 mg, 0.29 mmol) was reacted with the crude material obtained above and NaCNBH₃ (42 mg, 0.67 mmol) in 15 mL EtOH. The crude product was purified by preparative HPLC, and lyophilized to give the product as a solid (60 mg, 46%): 1H NMR (400 MHz, DMSO-D6) δ ppm 3.17 (t, J=6.95 Hz, 2 H) 3.54-3.62 (m, 2 H) 6.58-6.67 (m, 1 H) 7.18 (d, J=2.02 Hz, 1 H) 7.29-7.35 (m, 1 H) 7.42-7.49 (m, 2 H) 7.56 (dd, J=6.44, 2.65 Hz, 1 H) 8.38 (s, 1 H) 9.55 (s, 1 H); HRMS (ESI+) calcd for C₁₉H₁₃Cl₂FN₈ (MH+) 443.06970, found 443.0702.

### Example 201

### 8-chloro-4-[(3-chloro-4-fluorophenyl)amino]-6-{[(1-methyl-1H-imidazol-4-yl)methyl]amino}quinoline-3-carbonitrile

Following the procedure described above in Example 4, 6-amino-8-chloro-4-(3-chloro-4-fluorophenylamino)quinoline-3-carbonitrile (80 mg, 0.23 mmol) was reacted with 1-methyl-1H-imidazole-4-carbaldehyde (28 mg, 0.25 mmol) and NaCNBH₃ (22 mg, 0.35 mmol) in 5 mL EtOH. The crude product was purified by preparative HPLC, and lyophilized to give the product as a solid (50 mg, 49%): 1H NMR (400 MHz, DMSO-D6) δ ppm 3.61 (s, 3 H) 4.21 (d, J=5.31 Hz, 2 H) 6.68 (d, 1 H) 7.06 (s, 1 H) 7.20 (s, 1 H) 7.25-7.33 (m, 1 H) 7.45 (t, J=8.72 Hz, 1 H) 7.51-7.62 (m, 2 H) 8.37 (s, 1 H) 9.48 (s, 1 H); HRMS (ESI+) calcd for C₂₁H₁₅Cl₂FN₆ (MH+) 441.07920, found 441.0809.

### Example 202

### 8-(allyloxy)-4-[(3-chloro-4-fluorophenyl)amino]-6-nitroquinoline-3-carbonitrile

To a mixture of 4-(3-chloro-4-fluorophenylamino)-8-hydroxy-6-nitroquinoline-3-carbonitrile (274 mg, 0.77 mmol) and potassium carbonate (218 mg, 1.58 mmol) in DMF (7 mL) under nitrogen atmosphere was added allyl bromide (0.073 mL, 0.84 mmol) at RT. After 12 hr of reaction, the reaction was purified via preparative HPLC to give the desired product as a solid (229 mg, 75%): 1H NMR (400 MHz, DMSO-D6) δ ppm 4.92 (d, J=5.31 Hz, 2 H) 5.36 (dd, J=10.48,1.64 Hz, 1 H) 5.49-5.57 (m, 1 H) 6.11-6.23 (m, 1 H) 7.36 (s, 1 H) 7.49 (t, J=8.97 Hz, 1 H) 7.62 (s, 1 H) 7.95 (d, J=2.27 Hz, 1 H) 8.68 (s, 1 H) 9.08 (s, 1 H) 10.45 (s, 1 H).

### Example 203

### 4-[allyl(3-chloro-4-fluorophenyl)amino]-8-(allyloxy)-6-nitroquinoline-3-carbonitrile

The product was isolated from the synthesis of 8-(allyloxy)-4-[(3-chloro-4-fluorophenyl)amino]-6-nitroquinoline-3-carbonitrile in Example 202: 1H NMR (400 MHz, DMSO-D6) δ ppm 4.89 (d, J=5.81 Hz, 2 H) 5.05 (d, J=4.80 Hz, 2 H) 5.11-5.22 (m, 2 H) 5.39 (d, J=10.61 Hz, 1 H) 5.50 (dd, J=17.31, 1.64 Hz, 1 H) 6.00-6.18 (m, 2 H) 6.85-6.88 (m, 1 H) 7.09 (dd, J=6.57, 2.53 Hz, 1 H) 7.31 (t, J=9.09 Hz, 1 H) 7.99 (d, J=2.53 Hz, 1 H) 8.30 (s, 1 H) 8.82 (d, J=2.53 Hz, 1 H).

### Example 204

### 8-chloro-4-[(3-chloro-4-fluorophenyl)amino]-6-[(4,5-dihydro-1H-imidazol-2- ylmethyl)amino]quinoline-3-carbonitrile

A mixture of 6-amino-8-chloro-4-(3-chloro-4-fluorophenylamino)quinoline-3-carbonitrile (90 mg, 0.26 mmol) and 2-(chloromethyl)-4,5-dihydro-1H-imidazole hydrochloride (20 mg, 0.13 mmol) in ethanol (5 mL) in microwave reactor was heated to 180° C. for 2 h. The reaction was stripped to dryness and purified via preparative HPLC to give the desired product as a solid (12 mg, 11 %): 1H NMR (400 MHz, DMSO-D6) δ ppm 3.60 (s, 4 H) 4.12 (d, J=3.79 Hz, 2 H) 6.86 (t, J=4.04 Hz, 1 H) 7.25 (d, J=1.01 Hz, 1 H) 7.27-7.32 (m, 1 H) 7.45 (t, J=8.97 Hz, 1 H) 7.54 (dd, J=6.57, 2.53 Hz, 1 H) 7.60 (d, J=2.27 Hz, 1 H) 8.32 (s, 1 H) 8.39-8.43 (m, 1 H); HRMS (ESI+) calcd for C₂₀H₁₅Cl₂FN₆ (MH+) 429.07920, found 429.079.

### Example 205

### 8-chloro-4-[(3-chloro-4-fluorophenyl)amino]-6-({[4-(3-hydroxypropyl)-1H-imidazol-5-yl]methyl}amino)quinoline-3-carbonitrile

Hydrogenation of 1-(benzyloxymethyl)-4-(3-hydroxyprop-1-ynyl)-1H-imidazole-5-carbaldehyde (120 mg, 0.44 mmol) was carried out using parr shaker to give 1-(benzyloxymethyl)-4-(3-hydroxypropyl)-1H-imidazole-5-carbaldehyde in quantitative yield. The mixture of 1-(benzyloxymethyl)-4-(3-hydroxypropyl)-1H-imidazole-5-carbaldehyde (88 mg, 0.32 mmol), HCl (6N, 10 mL) and methanol (10 mL) was heated to reflux for 12 h. The reaction was stripped to dryness to give a crude material for further reaction without purification.

Following the procedure described in Example 4, 6-amino-8-chloro-4-(3-chloro-4-fluorophenylamino) quinoline-3-carbonitrile (100 mg, 0.29 mmol) was reacted with the crude material obtained above and NaCNBH₃ (22 mg, 0.35 mmol) in 15 mL EtOH. The crude product was purified by preparative HPLC, and lyophilized to give the product as a solid (25 mg, 18%): 1H NMR (400 MHz, DMSO-D6) δ ppm 1.64-1.75 (m, 2 H) 2.53-2.61 (m, 2 H) 3.33-3.41 (m, 2 H) 4.15 (d, J=4.04 Hz, 2 H) 6.52 (s, 1 H) 7.20 (s, 1 H) 7.25-7.35 (m, 1 H) 7.45 (t, J=8.97 Hz, 1 H) 7.50-7.56 (m,2 H) 7.60 (d, J=2.02 Hz, 1 H) 8.24 (s, 2 H) 8.38 (s, 1 H); HRMS (ESI+) calcd for C₂₃H₁₉Cl₂FN₆O (MH+) 485.10542, found 485.1053.

### Example 206

### N′-{8-chloro-4-[(3-chloro-4-fluorophenyl)amino]-3-cyanoquinolin-6-yl}-N,N-dimethylimidoformamide

The product was isolated from the reaction mixture of 6-amino-8-chloro-4-(3-chloro-4-fluorophenylamino)quinoline-3-carbonitrile with 2-iodo-2-methylpropane in DMF in the presence of silver carbonate (180° C.): 1H NMR (400 MHz, acetonitrile-D3) δ ppm 2.92 (s, 3 H) 2.98 (s, 3 H) 7.16-7.24 (m, 2 H) 7.35 (dd, J=6.44, 2.40 Hz, 1 H) 7.42 (d, J=2.02 Hz, 1 H) 7.59 (d, J=2.27 Hz, 1 H) 7.76 (s, 1 H) 7.96 (s,1 H) 8.43 (s, 1 H); HRMS (ESI+) calcd for C₁₉H₁₄Cl₂FN₅ (MH+) 402.06830, found 402.0682.

### Example 207

### 4-[(3-chloro-4-fluorophenyl)amino]-8-(2,3-dihydroxypropoxy)-6-nitroquinoline-3-carbonitrile

To a mixture of 8-(allyloxy)-4-(3-chloro-4-fluorophenylamino)-6-nitroquinoline-3-carbonitrile (138 mg, 0.35 mmol), reagent grade acetone (16 mL) and water (6 mL) was added 4-methylmorpholine N-oxide (235 mg, 2.01 mmol) followed by OsO₄ (0.5 mL, 2.5% in tBuOH). After 12 hr of reaction and workup (EtOAc/brine), the reaction was purified via preparative HPLC to give a solid product (71 mg, 47%): 1H NMR (400 MHz, DMSO-D6) δ ppm 2.07 (s, 1 H) 4.33 (d, J=5.56 Hz, 2 H) 5.03 (s, 2 H) 6.60 (s, 1 H) 6.83 (s, 1 H) 7.06 (s, 1 H) 7.19-7.26 (m, 2 H) 7.33-7.50 (m, 2 H) 7.70 (d, J=9.09 Hz, 1 H) 8.33 (s,1 H) 9.29 (s,1 H); HRMS (ESI+) calcd for C₁₉H₁₄ClFN₄O₅ (MH+) 433.07095, found 433.0705.

### Example 208

### 6-amino-4-[(3-chloro-4-fluorophenyl)amino]-8-(2,3-dihydroxypropoxy)quinoline-3- carbonitrile

To a 25 mL round-bottomed flask was added 4-(3-chloro-4-fluorophenylamino)-8-(2,3-dihydroxypropoxy)-6-nitroquinoline-3-carbonitrile (33 mg, 0.076 mmol), SnCl₂.2H₂O (104 mg, 0.48 mmol), and ethyl alcohol (8 mL). The mixture was heated to reflux for 12 hr. After cooling to RT, water (20 mL) was added followed by sodium carbonate to adjust pH to around 7. Workup (ethyl acetate/brine) of the reaction gave a solid as product in quantitative yield: 1H NMR (400 MHz, DMSO-D6) δ ppm 1.17 (t, J=7.07 Hz, 2 H) 3.50-3.53 (m, 1 H) 4.03 (q, J=6.91 Hz, 2 H) 4.72 (t, J=5.68 Hz, 1 H) 5.08 (d, J=4.80 Hz, 1 H) 5.73 (s, 2 H) 6.67-6.73 (m, J=2.02 Hz, 1 H) 6.78 (s, 1 H) 7.11 (dd, J=7.33, 3.79 Hz, 1 H) 7.27-7.33 (m, 1 H) 7.37 (t, J=9.09 Hz, 1 H) 8.30 (s, 1 H) 9.22 (s, 1 H); HRMS (ESI+) calcd for C₁₉H₁₆ClFN₄O₃ (MH+) 403.09677, found 403.0958.

### Example 209

### 4-[(3-chloro-4-fluorophenyl)amino]-8-(2,3-dihydroxypropoxy)-6-[(1H-imidazol-5-ylmethyl)amino]quinoline-3-carbonitrile

Following the procedure described above in Example 4, 6-amino-4-(3-chloro-4-fluorophenylamino)-8-(2,3-dihydroxypropoxy)quinoline-3-carbonitrile (33 mg, 0.08 mmol) was reacted with 4(5)-imidazole carboxaldehyde (0.014 g, 0.15 mmol) and NaCNBH₃ (7 mg, 0.11 mmol) in EtOH/THF (2mL/7 mL). The crude product was purified by preparative HPLC, and lyophilized to give the product as a solid (10 mg, 25%): 1H NMR (400 MHz, DMSO-D6) δ ppm 2.65-2.69 (m, 1 H) 3.49-3.55 (m, 2 H) 3.98 (dd, J=9.09, 6.32 Hz, 2 H) 4.08-4.13 (m, 1 H) 4.22-4.27 (m, 2 H) 4.72-4.77 (m, 1 H) 6.32-6.42 (m, 1 H) 6.82 (s, 1 H) 6.97 (s, 1 H) 7.03 (s, 1 H) 7.22 (d, J=9.09 Hz, 1 H) 7.41 (d, J=9.09 Hz, 2 H) 7.61 (s, 1 H) 8.25-8.34 (m, 2 H) 9.25 (s, 1 H); HRMS (ESI+) calcd for C₂₃H₂₀ClFN₆O₃ (MH+) 483.13422, found 483.1328.

### Example 210

### 6-[(2-azidoethyl)amino]-8-chloro-4-[(3-chloro-4-fluorophenyl)amino]quinoline-3- carbonitrile

Following the procedure described above in Example 4, 6-amino-8-chloro-4-(3-chloro-4-fluorophenylamino)quinoline-3-carbonitrile (0.258 g, 0.74 mmol) was reacted with 2-azidoacetaldehyde and NaCNBH₃ (22 mg, 0.35 mmol) in 20 mL EtOH. The crude product was purified by preparative HPLC, and lyophilized to give the product as a solid (13 mg, 4%): 1H NMR (400 MHz, DMSO-D6) δ ppm 4.91 (d, J=5.56 Hz, 2 H) 5.42 (t, J5.68 Hz, 2 H) 7.32-7.49 (m, 3 H) 7.56-7.63 (m, 1 H) 7.73-7.78 (m, 1 H) 7.83 (dd, J=7.96,1.14 Hz, 1 H); HRMS (ESI+) calcd for C₁₈H₁₂Cl₂FN₇ (MH+) 416.05880, found 416.0581.

### Example 211: 8-chloro-4-[(3-chloro-4-fluorophenyl)amino]-6-{[2-(1H-1,2,3-triazol-1-yl)ethyl]amino}quinoline-3-carbonitrile

To 6-(2-azidoethylamino)-8-chloro-4-(3-chloro-4-fluorophenylamino) quinoline-3-carbonitrile (65 mg, 0.16 mmol) in DMF (1.5 mL) under nitrogen was added trimethylsilylacetylene (2 mL), followed by CuSO₄.5H₂O (10 mg, 0.04 mmol) and sodium ascorbate (8 mg, 0.04 mmol) were added. After 12 hr, the reaction was worked-up (EtOAc extraction, wash with 1 N HCl 3×, brine 2×). The crude product was purified by preparative HPLC, and lyophilized to give the product as a solid (12 mg, 17%): 1H NMR (400 MHz, DMSO-D6) δ ppm 3.64-3.76 (m, 2 H) 4.63 (t, J=5.94 Hz, 2 H) 6.59-6.66 (m, 1 H) 7.17 (d, J=2.02 Hz, 1 H) 7.28-7.36 (m, 1 H) 7.44-7.50 (m, 2 H) 7.56 (dd, J=6.69, 2.65 Hz, 2 H) 7.73 (d, J=1.01 Hz, 1 H) 8.13 (s, 1 H) 8.39 (s, 1 H); HRMS (ESI+) calcd for C₂₀H₁₄Cl₂FN₇ (MH+) 440.05990, found 440.0609.

### Example 212

### 8-chloro-4-[(3-chloro-4-fluorophenyl)amino]-6-{[2-(1H-imidazol-1-yl)ethyl]amino}quinoline-3-carbonitrile

Step 1: A mixture of 2-bromo-1 ,1-diethoxyethane (1.0 mL, 6.65 mmol) and imidazole sodium salt (480 mg, 5.33 mmol) in DMF (4.5 mL) under nitrogen atmosphere was heated to 115° C. for 12 hr. Workup with EtOAc/brine gave 1-(2,2-diethoxyethyl)-1H-imidazole as liquid (423 mg, 43%).

Step 2: A mixture of 1-(2,2-diethoxyethyl)-1H-imidazole (222 mg, 1.21 mmol), HCl (˜1.25N in MeOH, 15 mL) and H₂O (0.5 mL) was taken to reflux temperature. The reaction was stripped to dryness after 3 hr of reaction. The crude material obtained was used for further reaction without purification.

Step 3: Following the procedure described above in Example 4, 6-amino-8-chloro-4-(3-chloro-4-fluorophenylamino)quinoline-3-carbonitrile (98 mg, 0.28 mmol) was reacted with the crude material obtained above and NaCNBH₃ (22 mg, 0.35 mmol) in 25 mL EtOH. The crude product was purified by preparative HPLC, and lyophilized to give the product as a solid (43 mg, 35%): 1H NMR (400 MHz, DMSO-D6) δ ppm 3.52 (q, J=6.40 Hz, 2 H) 4.20 (t, J=6.06 Hz, 2 H) 6.63 (t, J=5.68 Hz, 1 H) 6.88 (s, 1 H) 7.12 (d, J=2.27 Hz, 1 H) 7.21 (s, 1 H) 7.28-7.34 (m, 1 H) 7.43-7.51 (m, 2 H) 7.54 (dd, J=6.44, 2.40 Hz, 1 H) 7.63 (s, 1 H) 8.17 (s, 1 H) 8.39 (s, 1 H); HRMS (ESI+) calcd for C₂₁H₁₅Cl₂FN₆ (MH−) 439.06465,found 439.0661.

### Example 213

### 8-chloro-4-[(3-chloro-4-fluorophenyl)amino]-6-({2-[4-(2-hydroxyethyl)-1H-1,2,3-triazol-1-yl]ethyl}amino)quinoline-3-carbonitrile

Step 1: A mixture of 2-bromo-1,1-diethoxyethane (1.35 mL, 8.97 mmol) and sodium azide (885 mg, 13.6 mmol) in DMF (10 mL) under nitrogen was heated to 115° C. for 24 hr. After workup (EtOAc/brine), 2-azido-1,1-diethoxyethane (1.15 g, 81%) was obtained as a viscous liquid.

Step 2: To a mixture of 2-azido-1,1-diethoxyethane (96 mg, 0.60 mmol), CuSO₄.5H₂O (20 mg, 0.08 mmol) and sodium ascorbate (60 mg, 0.30 mmol) in water (4.5 mL) was added but-3-yn-1-ol (0.050 mL, 0.66 mmol) followed by tert-butanol (3 mL). After 4 hr of reaction and workup, 2-(1-(2,2-diethoxyethyl)-1H-1,2,3-triazol-4-yl)ethanol was obtained as a liquid (65 mg, 47%).

Step 3: A mixture of 2-(1-(2,2-diethoxyethyl)-1H-1,2,3-triazol-4-yl)ethanol (65 mg, 0.28 mmol), HCl (˜1.25N in MeOH, 15 mL) and H₂O (0.5 mL) was taken to reflux temperature. The reaction was stripped to dryness after 3 hr of reaction. The crude material obtained was used for further reaction without purification.

Step 4: Following the procedure described above in Example 4, 6-amino-8-chloro-4-(3-chloro-4-fluorophenylamino)quinoline-3-carbonitrile (59 mg, 0.17 mmol) was reacted with the crude material obtained in Step 3 and NaCNBH₃ (22 mg, 0.35 mmol) in 8 mL EtOH. The crude product was purified by preparative HPLC, and lyophilized to give the product as a solid (7 mg, 8%): 1H NMR (400 MHz, DMSO-D6) δ ppm 2.74 (t, J=7.07 Hz, 2 H) 3.59 (t, J=6.95 Hz, 2 H) 3.66 (q, J=6.32 Hz, 2 H) 4.55 (t, J=6.06 Hz, 2 H) 4.70 (s, 1 H) 6.62 (s, 1 H) 7.19 (d, J=2.27 Hz, 1 H) 7.30 (d, J=6.06 Hz, 1 H) 7.42-7.50 (m, 2 H) 7.55 (s, 1 H) 7.89 (s, 1 H) 8.37 (s, 1 H); HRMS (ESI+) calcd for C₂₂H₁₈Cl₂FN₇O (MH+) 486.10067, found 486.0996.

### Example 214

### 8-chloro-4-[(3-chloro-4-fluorophenyl)amino]-6-{[(4-isopropyl-1H-imidazol-5- yl)methyl]amino}quinoline-3-carbonitrile

Following the procedure described above in Example 4, 6-amino-8-chloro-4-(3-chloro-4-fluorophenylamino)quinoline-3-carbonitrile (76 mg, 0.22 mmol) was reacted with 4-isopropyl-1H-imidazole-5-carbaldehyde (36 mg, 0.26 mmol) and NaCNBH₃ (22 mg, 0.35 mmol) in 9 mL EtOH. The crude product was purified by preparative HPLC, and lyophilized to give the product as a solid (74 mg, 72%): 1H NMR (400 MHz, DMSO-D6) δ ppm 1.16 (d, J=7.07 Hz, 6 H) 4.16 (d, J=4.55 Hz, 2 H) 6.54 (d, J=4.42 Hz, 1 H) 7.19 (d, J=2.02 Hz, 1 H) 7.26-7.34 (m, 1 H) 7.45 (t, J=8.97 Hz, 1 H) 7.49-7.56 (m, 2 H) 7.61 (d, J=2.27 Hz, 1 H) 8.16 (s, 1 H) 8.39 (s, 1 H) 9.47 (s, 1 H); HRMS (ESI+) calcd for C₂₃H₁₉Cl₂FN₆ (MH+) 469.11050, found 469.1096.

### Example 215

### 6-{[(1-benzyl-1H-1,2,3-triazol-4-yl)methyl]amino}-8-chloro-4-[(3-chloro-4-fluorophenyl)amino]quinoline-3-carbonitrile

Following the procedure described above in Example 4, 6-amino-8-chloro-4-(3-chloro-4-fluorophenylamino)quinoline-3-carbonitrile (55 mg, 0.16 mmol) was reacted with 1-benzyl-1H-1,2,3-triazole-4-carbaldehyde (61 mg, 0.33 mmol) and NaCNBH₃ (22 mg, 0.35 mmol) in 6 mL EtOH. The crude product was purified by preparative HPLC, and lyophilized to give the product as a solid (30 mg, 36%): 1H NMR (400 MHz, DMSO-D6) δ ppm 4.45 (d, J=5.81 Hz, 2 H) 5.57 (s, 2 H) 6.89 (d, J=2.02 Hz, 1 H) 7.23-7.36 (m, 6 H) 7.41-7.56 (m, 3 H) 8.09 (s, 1 H) 8.38 (s, 1 H) 9.48 (s, 1 H); HRMS (ESI+) calcd for C₂₆H₁₈Cl₂FN₇ (MH+) 518.10575, found 518.1065.

### Example 216

### 6-([1,2,3]triazolo[1,5-a]pyridin-3-ylmethylamino)-8-chloro-4-(3-chloro-4-fluorophenylamino)quinoline-3-carbonitrile

Following the procedure described above in Example 4, 6-amino-8-chloro-4-(3-chloro-4-fluorophenylamino)quinoline-3-carbonitrile (51 mg, 0.15 mmol) was reacted with [1,2,3]triazolo[1,5-a]pyridine-3-carbaldehyde (25 mg, 0.17 mmol) and NaCNBH₃ (22 mg, 0.35 mmol) in 6 mL EtOH. The crude product was purified by preparative HPLC, and lyophilized to give the product as a solid (6 mg, 9%): 1H NMR (400 MHz, DMSO-D6) δ ppm 4.79 (d, J=5.31 Hz, 2 H) 7.02 (s, 1 H) 7.15 (t, J=6.69 Hz, 1 H) 7.25 (d, J=8.59 Hz, 1 H) 7.29-7.37 (m, 1 H) 7.37-7.52 (m, 3 H) 7.55 (d, J=1.52 Hz, 1 H) 8.02 (d, J=9.09 Hz, 1 H) 8.36-8.49 (m, 2 H) 9.03 (d, J=6.82 Hz, 1 H); HRMS (ESI+) calcd for C₂₃H₁₄Cl₂FN₇ (MH+) 478.07445, found 478.0757.

### Example 217

### 8-chloro-4-[(3-chloro-4-fluorophenyl)amino]-6-{[(1-methyl-1H-1,2,3-triazol-4-yl)methyl]amino}quinoline-3-carbonitrile

Following the procedure described above in Example 4, 6-amino-8-chloro-4-(3-chloro-4-fluorophenylamino)quinoline-3-carbonitrile (101 mg, 0.29 mmol) was reacted with 1-methyl-1H-1,2,3-triazole-4-carbaldehyde (40 mg, 0.36 mmol) and NaCNBH₃ (22 mg, 0.35 mmol) in 7 mL EtOH. The crude product was purified by preparative HPLC, and lyophilized to give the product as a solid (23 mg, 18%): 1H NMR (400 MHz, DMSO-D6) δ mppm 4.02 (s, 3 H) 4.44 (d, J=5.56 Hz, 2 H) 6.88 (t, J=5.68 Hz, 1 H) 7.23-7.35 (m, 2 H) 7.46 (t, J=9.09 Hz, 1 H) 7.51-7.59 (m, 2 H) 7.99 (s, 1 H) 8.39 (s, 1 H) 9.50 (s, 1 H); HRMS (ESI+) calcd for C₂₀H₁₄Cl₂FN₇ (MH+) 442.07445, found 442.074.

### Example 218

### N-(2-(4-((8-bromo-4-(3-chloro-4-fluorophenylamino)-3-cyanoquinolin-6-ylamino)methyl)-1H-1,2,3-triazol-1-yl)ethyl)-2-methoxyacetamide

Step 1: A mixture of 1-(3-dimethylaminopropyl)-3-ethylcarbodiimide hydrochloride (5 g, 26.08 mmol), 4-dimethylaminopyridine (1.76 g, 14.41 mmol), 2-chloroethanamine hydrochloride (1.41 g,12.16 mmol),2-methoxyacetic acid (1 mL, 13.03 mmol) in DMF (10 mL) was allowed to react for 12 hr. After workup, N-(2-chloroethyl)-2-methoxyacetamide (0.711 g, 39%) was obtained as a viscous liquid: 1H NMR (400 MHz, DMSO-D6) δ ppm 3.31 (s, 3 H) 3.42 (q, J=6.23 Hz, 2 H) 3.63 (t, J=6.44 Hz, 2 H) 3.82 (s, 2 H) 8.01 (s, 1 H).

Step 2: A mixture of N-(2-chloroethyl)-2-methoxyacetamide (315 mg, 2.09 mmol) and sodium azide (237 mg, 3.65 mmol) in DMF (5 mL) in microwave reactor was heated to 100° C. for 1 h. After workup (EtOAc/brine), N-(2-azidoethyl)-2-methoxyacetamide was obtained in quantitative yield: 1H NMR (400 MHz, DMSO-D6) δ ppm 3.27-3.33 (m, 5 H) 3.35-3.41 (m, 2 H) 3.80 (s, 2 H) 8.01 (s, 1 H).

Step 3: To a mixture of N-(2-azidoethyl)-2-methoxyacetamide (201 mg, 1.27 mmol), CuSO₄.5H₂O (45 mg, 0.1 8 mmol) and sodium ascorbate (100 mg, 0.51 mmol) in water (10 mL) was added 3,3-diethoxyprop-1-yne (0.2 mL, 1.40 mmol) followed by tert-butanol (10 mL). After 4 hr of reaction and workup, N-(2-(4-(diethoxymethyl)-1H-1,2,3-triazol-1-yl)ethyl)-2-methoxyacetamide was obtained as a solid (118 mg, 32%).

Step 4: A mixture of N-(2-(4-(diethoxymethyl)-1H-1,2,3-triazol-1-yl)ethyl)-2-methoxyacetamide (118 mg, 0.41 mmol), HCl (˜1.25N in MeOH, 15 mL) and H₂O (0.5 mL) was taken to reflux temperature. The reaction was stripped to dryness after 3 hr of reaction. The crude material obtained was used for further reaction without purification.

Step 5: Following the procedure described above in Example 4, 6-amino-8-bromo-4-(3-chloro-4-fluorophenylamino)quinoline-3-carbonitrile (50 mg, 0.13 mmol, prepared as deacribed in Example 78) was reacted with the crude material obtained in Step 2 and NaCNBH₃ (11 mg, 0.18 mmol) in 5 mL EtOH. The crude product was purified by preparative HPLC, and lyophilized to give the product as a solid (17 mg, 23%): 1H NMR (400 MHz, DMSO-D6) δ ppm 3.21 (s, 3 H) 3.53 (q, J=5.89 Hz, 2 H) 3.69 (s, 2 H) 4.40-4.49 (m, 4 H) 6.88 (t, J=5.56, 5.56 Hz, 1 H) 7.27-7.33 (m, 2 H) 7.46 (t, J=8.97 Hz, 1 H) 7.54 (dd, J=6.57, 2.53 Hz, 1 H) 7.76 (d, J=2.53 Hz, 1 H) 7.94 (t, J=5.56 Hz, 1 H) 7.99 (s, 1 H) 8.39 (s, 1 H); HRMS (ESI+) calcd for C₂₄H₂₁BrClFN₈O₂ (MH+) 587.07161, found 587.0725.

### Example 219

### N-(2-(4-((8-chloro-4-(3-chloro-4-fluorophenylamino)-3-cyanoquinolin-6-ylamino)methyl)-1H-1,2,3-triazol-1-yl)ethyl)-2-methoxyacetamide

Following the procedure described above in Example 4, 6-amino-8-chloro-4-(3-chloro-4-fluorophenylamino)quinoline-3-carbonitrile (50 mg, 0.14 mmol) was reacted with the crude material obtained in step 4 of Example 218 and NaCNBH₃ (11 mg, 0.18 mmol) in 5 mL EtOH. The crude product was purified by preparative HPLC, and lyophilized to give the product as a solid (10 mg, 13%): 1H NMR (400 MHz, DMSO-D6) δ ppm 3.21 (s, 3 H) 3.53 (q, J=5.81 Hz, 2 H) 3.69 (s, 2 H) 4.44 (t, J=5.68 Hz, 4 H) 6.54 (t, 1 H) 6.88 (t, J=5.94 Hz, 1 H) 7.26 (d, J=2.53 Hz, 1 H) 7.27-7.34 (m, 1 H) 7.46 (t, J=8.97 Hz, 1 H) 7.53-7.57 (m, 2 H) 7.94 (t, J=5.56 Hz, 1 H) 8.00 (s, 1 H) 8.39 (s, 1 H); HRMS (ESI+) calcd for C₂₄H₂₁Cl₂FN₈O₂ (MH+) 543.12213, found 543.1222.

### Example 220

### 8-bromo-4-[(3-chloro-4-fluorophenyl)amino]-6-{[(1-methyl-1H-imidazol-2- yl)methyl]amino}quinoline-3-carbonitrile

Following the procedure described above in Example 4, 6-amino-8-bromo-4-(3-chloro-4-fluorophenylamino)quinoline-3-carbonitrile (134 mg, 0.34 mmol, prepared as described in Example 78) was reacted with 1-methyl-1H-imidazole-2-carbaldehyde (70 mg, 0.64 mmol) and NaCNBH₃ (22 mg, 0.35 mmol) in 6 mL EtOH. The crude product was purified by preparative HPLC, and lyophilized to give the product as a solid (26 mg, 16%): 1H NMR (400 MHz, DMSO-D6) δ ppm 3.64 (s, 3 H) 4.40 (d, J=5.05 Hz, 2 H) 6.78-6.88 (m, 1 H) 7.15 (d, J=11.01 Hz, 1 H) 7.24-7.34 (m, 2 H) 7.45 (t, J=9.09 Hz, 1 H) 7.53 (dd, J=6.57, 2.53 Hz, 1 H) 7.83 (d, J=2.27 Hz, 1 H) 8.14 (s, 1 H) 8.41 (s, 1 H) 9.49 (s, 1 H); HRMS (ESI+) calcd for C₂₁H₁₅BrClFN₆ (MH+) 485.02869, found 485.0306.

### Example 221

### 8-bromo-4-(3-chloro-4-fluorophenylamino)-6-((1-methyl-1H-1,2,3-triazol-4-yl)methylamino)quinoline-3-carbonitrile

Following the procedure described above in Example 4, 6-amino-8-bromo-4-(3-chloro-4-fluorophenylamino)quinoline-3-carbonitrile (58 mg, 0.15 mmol, prepared as described in Example 78) was reacted with 1-methyl-1H-1,2,3-triazole-4-carbaldehyde (54 mg, 0.49 mmol) and NaCNBH₃ (22 mg, 0.35 mmol) in 7 mL EtOH. The crude product was purified by preparative HPLC, and lyophilized to give the product as a solid (15 mg, 21%): 1H NMR (400 MHz, DMSO-D6) δ ppm 4.01 (s, 3 H) 4.44 (d, J=5.31 Hz, 2 H) 6.87 (s, 1 H) 7.31 (d, J=2.27 Hz, 2 H) 7.45 (t, J=9.09 Hz, 1 H) 7.53 (t, J=6.32 Hz, 1 H) 7.74 (d, J=11.77 Hz, 1 H) 7.98 (s, 1 H) 8.38 (s, 1 H) 9.49 (s, 1 H); HRMS (ESI+) calcd for C₂₀H₁₄BrClFN₇ (M H+) 486.02394, found 486.0244.

### Example 222

### 8-chloro-4-(3-chloro-4-fluorophenylamino)-6-((1-(2-(2-oxooxazolidin-3-yl)ethyl)-1H-1,2,3-triazol-4-yl)methylamino)quinoline-3-carbonitrile

Step 1: To a mixture of 3-(2-azidoethyl)oxazolidin-2-one (492 mg, 3.15 mmol), CuSO₄.5H₂O (55 mg, 0.22 mmol) and sodium ascorbate (77 mg, 0.39 mmol) in DMF (1 OmL) was added 3,3-diethoxyprop-1-yne (0.675 mL, 4.74 mmol) followed by tert-butanol (3 mL). After 12 hr of reaction and workup, 3-(2-(4-(diethoxymethyl)-1H-1,2,3-triazol-1-yl)ethyl)oxazolidin-2-one was obtained as a solid.

Step 2: A mixture of 3-(2-(4-(diethoxymethyl)-1H-1,2,3-triazol-1-yl)ethyl)oxazolidin-2-one obtained in Step 1, HCl (˜1.25N in MeOH, 8 mL) and H₂O (0.5 mL) was taken to reflux temperature. The reaction was stripped to dryness after 3 hr of reaction. The crude material obtained was used for further reaction without purification.

Step 3: Following the procedure described above in Example 4, 6-amino-8-chloro-4-(3-chloro-4-fluorophenylamino)quinoline-3-carbonitrile (64 mg, 0.18 mmol) was reacted with the crude material obtained in Step 2 and NaCNBH₃ (11 mg, 0.18 mmol) in 5 mL EtOH. The crude product was purified by preparative HPLC, and lyophilized to give the product as a solid (6 mg, 6%): 1H NMR (400 MHz, DMSO-D6) δ ppm 3.35-3.41 (m, 2 H) 3.54-3.60 (m, 2 H) 4.08-4.14 (m, 2 H) 4.46 (d, J=5.56 Hz, 2 H) 4.50-4.55 (m, 2 H) 6.61 (s, 1 H) 6.91 (t, J=6.19 Hz, 1 H) 7.24-7.32 (m, 2 H) 7.45 (t, J=8.97 Hz, 1 H) 7.51-7.57 (m, 2 H) 8.06 (s, 1 H) 8.39 (s, 1 H) 9.48 (s, 1 H); HRMS (ESI+) calcd for C₂₄H₁₉Cl₂FN₈O₂ (MH+) 541.10648, found 541.1068.

### Example 223

### 8-bromo-4-(3-chloro-4-fluorophenylamino)-6-((1-(2-(2-oxooxazolidin-3-yl)ethyl)-1H-1,2,3-triazol-4-yl)methylamino)quinoline-3-carbonitrile

Following the procedure described above in Example 4, 6-amino-8-bromo-4-(3-chloro-4-fluorophenylamino)quinoline-3-carbonitrile (53 mg, 0.14 mmol, prepared as described in Example 78) was reacted with the crude material obtained in Example 235, Step 2, and NaCNBH₃ (11 mg, 0.18 mmol) in 5 mL EtOH. The crude product was purified by preparative HPLC, and lyophilized to give the product as a solid (8 mg, 10%): 1H NMR (400 MHz, DMSO-D6) δ ppm 3.34-3.40 (m, 2 H) 3.53-3.59 (m, 2 H) 4.10 (dd, J=8.72, 7.20 Hz, 2 H) 4.46 (d, J=5.81 Hz, 2 H) 4.50-4.55 (m, 2 H) 6.90 (s, 1 H) 7.30 (d, J=2.02 Hz, 2 H) 7.44 (t, J=8.97 Hz, 1 H) 7.51 (s, 1 H) 7.74 (s, 1 H) 8.06 (s, 1 H) 8.37 (s, 1 H) 8.45 (s, 1 H); HRMS (ESI+) calcd for C₂₄H₁₉BrClFN₈O₂ (MH+) 585.05596, found 585.0571.

### Example 224

### 8-chloro-4-(3-chloro-4-fluorophenylamino)-6-((1-(morpholinosulfonylmethyl)-1H-1,2,3-triazol-4-yl)methylamino)quinoline-3-carbonitrile

Step 1: A mixture of 4-(chloromethylsulfonyl)morpholine (505 mg, 2.54 mmol) and sodium azide (400 mg, 6.15 mmol) in DMF (10 mL) was heated to 120° C. for 24 h. After workup (EtOAc/brine), 4-(azidomethylsulfonyl)morpholine was obtained a white solid (419 mg, 80%).

Step 2: To a mixture of 4-(azidomethylsulfonyl)morpholine (370 mg, 1.80 mmol), CuSO₄.5H₂O (40 mg, 0.16 mmol) and sodium ascorbate (55 mg, 0.28 mmol) in water (10 mL) was added 3,3-diethoxyprop-1-yne (0.39 mL, 2.74 mmol) followed by tert-butanol (3 mL). After 12 h of reaction and workup, 4-((4-(diethoxymethyl)-1H-1,2,3-triazol-1-yl)methylsulfonyl)morpholine was obtained as a solid.

Step 3: A mixture of 4-((4-(diethoxymethyl)-1H-1,2,3-triazol-1-yl)methylsulfonyl)morpholine obtained above, HCl (˜1.25N in MeOH, 15 mL) and H₂O (0.5 mL) was taken to reflux temperature. The reaction was stripped to dryness after 3 hr of reaction. The crude material obtained was used for further reaction without purification.

Step 4: Following the procedure described above in Example 4, 6-amino-8-chloro-4-(3-chloro-4-fluorophenylamino)quinoline-3-carbonitrile (55 mg, 0.16 mmol) was reacted with the crude material obtained in Step 3 and NaCNBH₃ (11 mg, 0.18 mmol) in 8 mL EtOH. The crude product was purified by preparative HPLC, and lyophilized to give the product as a solid (27 mg, 29%): 1H NMR (400 MHz, DMSO-D6) δ ppm 2.84-2.95 (m, 4 H) 3.35-3.43 (m, 4 H) 4.54 (d, J=6.06 Hz, 2 H) 6.09 (s, 2 H) 7.02 (t, J=6.32 Hz, 1 H) 7.27-7.34 (m, 2 H) 7.48 (t, J=9.09 Hz, 1 H) 7.51-7.58 (m, 2 H) 8.14 (s, 1 H) 8.37 (s, 1 H) 9.50 (s, 1 H); HRMS (ESI+) calcd for C₂₄H₂₁Cl₂FN₈O₃S (MH+) 591.08912, found 591.0899.

### Example 225

### 4-[(3-Chloro-4-fluorophenyl)amino]-7-methyl-6-[(pyridin-3-ylmethyl)amino]quinoline-3-carbonitrile

Step 1: A 300 mL round-bottomed flask was charged with 3-methyl-4-nitro-phenylamine (8.0 g, 52.6 mmol), ethyl (ethoxymethylene)cyanoacetate (9.8 g, 57.8 mmol) and 40 mL DMF. The mixture was stirred vigorously to dissolve both reagents, Cs₂CO₃ (34.3 g, 105.2 mmol) was added, and the reaction mixture was stirred at RT for 2 hours. To work up, the contents of the flask were poured into 600 mL water and the precipitate collected by suction filtration, washed three times with water, then washed twice with ether, and dried under vacuum to give ethyl-2-cyano-3-[(3-methyl-4-nitrophenyl)amino]acrylate as a yellow solid (14.4 g, 99% yield): 1H NMR (400 MHz, DMSO-D6) δ ppm (66%) 1.20-1.35 (m, 3 H) 2.53-2.61 (m, 3 H) 4.14-4.33 (m, 2 H) 7.41-7.49 (m, J=9.09, 2.53 Hz, 1 H) 7.53 (d, J=2.53 Hz, 1 H) 8.05 (s, 1H) 8.45 (s, 1 H) 11.01 (s, 1 H); (34%) 1.17-1.36 (m, 3 H) 2.53-2.59 (m, 3 H) 4.16-4.32 (m, 2 H) 7.54-7.58 (m, 1 H) 7.66 (d, J=2.27 Hz, 1 H) 8.07 (s,1 H) 8.60 (d, J=12.88 Hz, 1 H) 10.82 (d, J=13.39 Hz, 1 H); HRMS (ESI+) calcd for C₁₃H₁₃N₃O₄ 276.09788, found (MH+), 276.0978.

Step 2: In a 2L 3-necked round-bottomed flasks equipped with a stir bar, ethylene glycol/water cooled condenser, heating mantle, inert gas inlet/outlet and an internal temperature monitor, ethyl-2-cyano-3-[(3-methyl-4-nitrophenyl)amino]acrylate (14.0 g, 51.0 mmol) was suspended in 570 mL Dowtherm A. Argon or nitrogen was bubbled through suspension for 30 min. The flask was then heated to 260° C. for 4.5 hours under inert gas. The reaction was then stirred at RT overnight. The contents of the flask were poured into 800 mL hexane, stirred vigorously and filtered. The resulting brown precipitate was washed twice with hexanes and twice with dichloromethane and dried under vacuum. The product was isolated as brown powder (a mixture of two regioisomers (7-methyl-6-nitro-4-oxo-1,4-dihydro-quinoline-3-carbonitrile and 5-methyl-6-nitro-4-oxo-1,4-dihydro-quinoline-3-carbonitrile) and was used in the next step without further separation (6.7 g, 57% yield).

Step 3: In a 100 mL round-bottomed flask equipped with a condenser, the products from the previous step (3.5 g, 15.3 mmol) were taken up in 25 mL POCl₃ and heated at reflux for 4 hours. The reaction mixture was then allowed to cool to RT, and the POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum overnight to give a dark brown solid as a mixture of two regioisomers (4-chloro-7-methyl-6-nitro-quinoline-3-carbonitrile and 4-chloro-5-methyl-6-nitro-quinoline-3-carbonitrile), used in the next step without further separation (3.02 g, 80% yield).

Step 4: In a 100 mL round-bottomed flask equipped with a condenser, the product from step 3 (0.8 g, 3.2 mmol) was taken up in 25 mL of EtOH, and 3-chloro-4-fluoroaniline (0.56 g, 3.9 mmol) was added in one portion. The reaction mixture was heated at reflux for 3.5 hours. The reaction mixture was then allowed to cool to RT and the EtOH was removed under reduced pressure. The residue was then partitioned between 30 mL ether and 25 mL saturated NaHCO₃, and stirred for 10 minutes, then filtered and dried under high vacuum overnight to give a brown-yellow solid as a mixture of two regioisomers (4-(3-Chloro-4-fluoro-phenylamino)-7-methyl-6-nitro-quinoline-3-carbonitrile and 4-(3-Chloro-4-fluoro-phenylamino)-5-methyl-6-nitro-quinoline-3-carbonitrile), and was used in the next step without further separation (0.42 g, 37% yield).

Step 5: In a 100 mL round-bottomed flask equipped with a condenser, the product from step 4 (0.42 g, 1.2 mmol) was taken up in 17 mL EtOH and tin chloride dihydrate (1.33 g, 5.89 mmol) was added. The reaction mixture was heated at reflux for 2.5 hours, until TLC analysis showed complete disappearance of the nitroquinoline. The reaction mixture was then cooled to RT and poured into ice water. The orange suspension was neutralized with saturated NaHCO₃ and extracted into CHCl₃ (3×100 mL), and the combined organic layers washed with brine, dried over anhydrous Na₂SO₄, filtered and evaporated. Evaporation of the CHCl₃ extracts gave a brown-yellow powder as a mixture of two regioisomers (6-amino-4-(3-chloro-4-fluoro-phenylamino)-7-methyl-quinoline-3-carbonitrile (HRMS (ESI+) calcd for C₁₇H₁₂ClFN₄ (MH+) 327.08073, found 327.081) and 6-amino-4-(3-chloro-4-fluoro-phenylamino)-5-methyl-quinoline-3-carbonitrile), and was used in the next step without further separation (0.24 g, 62% yield).

Step 6: Following the procedure described above in Example 4, (6-amino-4-(3-chloro-4-fluoro-phenylamino)-7 and 5-methyl-quinoline-3-carbonitrile (0.19 g, 0.58 mmol) was reacted with 3-pyridine carboxyaldehyde (0.19 g, 1.76 mmol) and NaCNBH₃ (71.4 mg, 1.13 mmol) in 4 mL EtOH. The crude product was purified by preparative HPLC, and lyophilized to give the product as a yellow solid (35.0 mg, 14%). The ¹H-NMR-NOE verified the identity of product as 4-[(3-chloro-4-fluorophenyl)amino]-7-methyl-6-[(pyridin-3-ylmethyl)amino]quinoline-3-carbonitrile:

1H NMR (400 MHz, DMSO-D6) δ ppm 2.38 (s, 3 H) 4.49 (d, J=5.56 Hz, 2 H) 6.30 (s, 1 H) 7.06 (s, 1 H) 7.10-7.20 (m, 1 H) 7.24-7.47 (m, 3 H) 7.59-7.80 (m, 2 H) 8.33 (s, 1 H) 8.38-8.46 (m, 1 H) 8.57 (s, 1 H) 9.25 (s, 1 H); HRMS (ESI+) calcd for C₂₃H₁₇ClFN₅ (MH+) 418.12293, found 418.1235.

### Example 226

### 4-[(3-chloro-4-fluorophenyl)amino]-6-[(1H-imidazol-5-ylmethyl)amino]-7-methylquinoline-3-carbonitrile

Following the procedure described above in Example 4, (6-amino-4-(3-chloro-4-fluoro-phenylamino)-7 and 5-methyl-quinoline-3-carbonitrile (0.18 g, 0.55 mmol) were reacted with 4(5)-imidazolecarboxyaldehyde (0.11 g, 1.1 mmol) and NaCNBH₃ (51.9 mg, 0.83 mmol) in 5 mL EtOH. The crude product was purified by preparative HPLC, and lyophilized to give the product as a yellow solid (21.0 mg, 9%): 1H NMR (400 MHz, DMSO-D6) δ ppm 2.34 (s, 3 H) 4.35 (d, J=5.31 Hz, 2 H) 5.65 (t, J=5.68 Hz, 1 H) 7.05 (s, 1 H) 7.17-7.30 (m, 2 H) 7.36-7.51 (m, 2 H), 7.63 (d, J=10.61 Hz, 2 H) 8.32 (s, 1 H) 9.35 (br, s, 1 H) 11.98 (br, s, 1 H); HRMS (ESI+) calcd for C₂₁H₁₆ClFN₆ (MH+) 407.11818, found 407.1185.

### Example 227

### 4-[(3-chloro-4-fluorophenyl)amino]-7-methyl-6-[(2-morpholin-4-ylethyl)amino]quinoline-3-carbonitrile

Following the procedure described above in Example 4, (6-amino-4-(3-chloro-4-fluoro-phenylamino)-7 and 5-methyl-quinoline-3-carbonitrile (0.20 g, 0.55 mmol), were reacted with NaCNBH₃ (57.5 mg, 0.83 mmol) and morpholin-4-yl-acetaldehyde (prepared by heating the corresponding dimethyl acetal (0.322 g, 1.84 mmol) in 2.0 mL concentrated HCl for 5 minutes in a microwave reactor at 110° C., then neutralizing with solid NaHCO₃ until pH=6) in 5 mL EtOH and 1.5 mL THF. The crude product was purified by preparative HPLC, and lyophilized to give the product as a yellow solid (38.0 mg, 14%): 1H NMR (400 MHz, DMSO-D6) δ ppm 2.31 (s, 3 H) 2.38-2.47 (m, 4 H) 2.62 (t, J=6.69 Hz, 2 H) 3.14-3.20 (m, J=2.27 Hz, 2 H) 3.54-3.64 (m, 4 H) 5.38 (t, J=5.18 Hz, 1 H) 7.04-7.11 (m, 1 H) 7.15-7.26 (m, 1 H) 7.37-7.46 (m, 2 H) 7.64 (s, 1 H) 8.33 (s, 1 H) 9.34 (s, 1 H); HRMS (ESI+) calcd for C₂₃H₂₃ClFN₅O (MH+) 440.16479, found 440.1654.

### Example 228

### 4-[(3-chloro-4-fluorophenyl)amino]-6-{[(2,4-dioxo-1,2,3,4-tetrahydropyrimidin-5-yl)methyl]amino}quinoline-3-carbonitrile

Following the procedure described above in Example 4, 6-amino-4-(3-chloro-4-fluoro-phenylamino)-quinoline-3-carbonitrile (0.15 g, 0.48 mmol) was reacted with 5-formyluracil (0.13 g, 0.96 mmol) and NaCNBH₃ (45.2 mg, 0.72 mmol) in 5 mL EtOH and 2.5 mL THF. The crude product was purified by preparative HPLC, and lyophilized to give the product as a yellow solid (58.5 mg, 28%): 1 H NMR (400 MHz, DMSO-D6) δ ppm 3.98 (d, J=5.05 Hz, 2 H) 6.43 (d, J=11.12 Hz, 1 H) 7.12 (d, J=2.27 Hz, 1 H) 7.23-7.29 (m, 1 H) 7.32 (dd, J=8.97, 2.40 Hz, 1 H) 7.36-7.51 (m, 3 H) 7.69 (d, J=9.09 Hz, 1 H) 8.30 (s, 1 H) 9.35 (s, 1 H) 10.77 (d, J=7.58 Hz, 1 H) 11.18 (s, 1 H); HRMS (ESI+) calcd for C₂₁H₁₄ClFN₆O₂ (MH+) 437.09235, found 437.0922.

### Example 229

### 4-[(3-chloro-4-fluorophenyl)amino]-6-[(1H-imidazol-5-ylmethyl)amino]-8-(trifluoromethyl)quinoline-3-carbonitrile

Step 1: In a 100 mL round-bottomed flask, 4-nitro-3-trifluoromethyl-phenylamine (3.0 g, 14.6 mmol) and ethyl (ethoxymethylene)cyanoacetate (2.71 g, 16 mmol) were dissolved in 15 mL DMF, and Cs₂CO₃ (9.5 g, 29.2 mmol) was added. The mixture was stirred at RT for 1.5 hours, and poured into 500 mL water. The yellow precipitate was collected by suction filtration, washed three times with water, and dried under vacuum to give ethyl-2-cyano-3-{[4-nitro-3-(trifluoromethyl)phenyl]amino}acrylate as a yellow solid (4.26 g, 89% yield): 1H NMR (400 MHz, DMSO-D6) δ ppm (68%) 1.16-1.37 (m, 3 H) 4.07-4.42 (m, 2 H) 7.93 (dd, J=8.97, 2.15 Hz, 1 H) 8.06 (d, J=2.27 Hz, 1 H) 8.21 (s, 1 H) 8.55 (s, 1 H) 11.16 (s, 1 H); (32%) 1.17-1.39 (m, 3 H) 4.15-4.34 (m, 2 H) 7.99-8.05 (m, 1 H), 8.19 (s, 1 H) 8.26 (d, J=1.77 Hz, 1 H) 8.68 (d, J=13.39 Hz, 1 H) 10.99 (d, J=13.90 Hz, 1 H); HRMS (ESI+) calcd for C₁₃H₁₀F₃N₃O₄ 330.06962, found (MH+) 330.0698.

Step 2: In a 1 L 3-necked round-bottomed flasks equipped with a stir bar, ethylene glycol/water cooled condenser, heating mantle, inert gas inlet/outlet and an internal temperature monitor, 2-cyano-3-(4-nitro-2-trifluoromethyl-phenylamino)-acrylic acid ethyl ester (8.5 g, 25.7 mmol) was suspended in 300 mL Dowtherm A. Argon was bubbled through suspension for 30 min. The flask was then heated to 260° C. for 8 hours under inert gas. They were then allowed to cool to RT and the contents of flask were poured into 500 mL hexane, stirred vigorously and filtered. The resulting brown precipitate was washed twice with hexanes and once with dichloromethane and dried under vacuum. The product 6-nitro-4-oxo-8-(trifluoromethyl)-1,4-dihydroquinoline-3-carbonitrile was isolated as a brown-yellow solid (6.0 g, 82%): 1H NMR (400 MHz, DMSO-D6) δ ppm 8.69 (s, 1 H) 8.73 (d, J=2.53 Hz, 1 H) 9.06 (d, J=2.78 Hz, 1 H); HRMS (ESI+) calcd for C₁₁H₄F₃N₃O₃ (MH+) 284.02775, found 284.0276.

Step 3: In a 100 mL round-bottomed flask equipped with a condenser, the product from the previous step (3.5 g, 12.4 mmol) was taken up in 25 mL POCl₃ and heated at reflux for 5 hours. The reaction mixture was then stirred at RT overnight, and the POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum to give a brown solid as 4-chloro-6-nitro-8-trifluoromethyl-quinoline-3-carbonitrile (3.5 g, 93% yield):

1H NMR (400 MHz, DMSO-D6) δ ppm 8.92-9.00 (m, J=1.77 Hz, 1 H) 9.26-9.34 (m, J=2.02 Hz, 1 H) 9.57 (s, 1 H).

Step 4: In a 100 mL round-bottomed flask equipped with a condenser, the product from step 3 (2.44 g, 8.1 mmol) was taken up in 35mL EtOH, and 3-chloro-4-fluoroaniline (1.41 g, 9.7 mmol) was added in one portion. The reaction mixture was heated at reflux for 1 hour and was stirred at RT overnight. The EtOH was removed under reduced pressure, the residue was then partitioned between 50 mL ether and 25 mL saturated NaHCO₃, and stirred for 15 minutes, then evaporated some ether by rotovamp until precipitate formed. The mixture was filtered and dried under high vacuum overnight to give a brown-yellow solid as 4-[(3-chloro-4-fluorophenyl)amino]-6-nitro-8-(trifluoromethyl)quinoline-3-carbonitrile (3.3 g, 99% yield): 1H NMR (400 MHz, DMSO-D6) δ ppm 7.37-7.48 (m, 1 H) 7.53 (t, J=8.97 Hz, 1 H) 7.71 (dd, J=6.44, 2.40 Hz, 1 H) 8.79 (d, J=2.27 Hz, 1 H) 8.87 (s, 1 H) 9.80 (d, J=2.27 Hz, 1 H) 10.77 (s, 1 H); HRMS (ESI+) calcd for C₁₇H₇ClF₄N₄O₂ (MH+) 411.02664, found 411.026.

Step 5: In a 100 mL round-bottomed flask equipped with a condenser, the product from step 4 (1.65 g, 4.0 mmol) was taken up in 50 mL EtOH and tin chloride dihydrate (4.53 g, 20.1 mmol) was added. The reaction mixture was heated at reflux for 1 hour, until LC/MS. analysis showed complete disappearance of the nitroquinoline. The reaction mixture was then cooled to RT and poured into ice water. The orange suspension was neutralized with saturated NaHCO₃ and extracted with CHCl₃ (3×150 mL) first, and then extracted with EtOAc (2×150 mL). The combined organic layers washed with brine, dried over anhydrous Na₂SO₄, filtered and evaporated. Evaporation of the organic extracts gave a yellow solid as 6-amino-4-[(3-chloro-4-fluorophenyl)amino]-8-(trifluoromethyl)quinoline-3-carbonitrile (1.5 g, 98% yield): 1H NMR (400 MHz, DMSO-D6) δ ppm 3.92-4.12 (m, 1 H) 6.09 (s, 2 H) 7.15-7.31 (m, 1 H) 7.33-7.53 (m, 2 H) 7.70 (d, J=2.27 Hz, 1 H) 8.44 (s, 1 H) 9.57 (s, 1 H); HRMS (ESI+) calcd for C₁₇H₉ClF₄N₄ (MH+) 381.05246, found 381.053.

Step 6: Following the procedure described above in Example 4, 6-amino-4-[(3-chloro-4-fluorophenyl)amino]-8-(trifluoromethyl)quinoline-3-carbonitrile (0.15 g, 0.39 mmol) was reacted with 4(5)-imidazolecarboxyaldehyde (75.7 mg, 0.79 mmol) and NaCNBH₃ (37.1 mg, 0.72 mmol) in 5 mL EtOH. The crude product was purified by preparative HPLC, and lyophilized to give the product as a yellow solid (137.2 mg, 76%): 1H NMR (400 MHz, DMSO-D6) δ ppm 4.31 (d, J=5.31 Hz, 2 H) 6.82-6.91 (m, J=5.31, 5.31 Hz, 1 H) 7.08 (s, 1 H) 7.28-7.37 (m, 1 H) 7.41-7.50 (m, 2 H) 7.57 (dd, J=6.69, 2.65 Hz, 1 H) 7.66 (d, J=1.01 Hz, 1 H) 7.86 (d, J=2.27 Hz, 1 H) 8.41 (s, 1 H) 9.55 (s, 1 H) 12.18 (s, 1 H); HRMS (ESI+) calcd for C₂₁H₁₃ClF₄N₆ (MH+) 461.08991, found 461.0903.

### Example 230

### 4-[(3-chloro-4-fluorophenyl)amino]-6-[(pyridin-3-ylmethyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with pyridine-3-carbaldehyde (36.6 mg, 0.34 mmol) and NaCNBH₃ (11.6 mg, 0.18 mmol) in 4 mL EtOH. The crude product was purified by preparative HPLC, and lyophilized to give the product as a yellow solid (58.0 mg, 47%): 1H NMR (400 MHz, DMSO-D6) δ ppm 4.47 (d, J=5.56 Hz, 2 H) 7.15-7.24 (m, 1 H) 7.25-7.33 (m, 1 H) 7.38 (dd, J=7.58, 4.80 Hz, 1 H) 7.41-7.49 (m, 2 H) 7.55 (dd, J=6.57, 2.53 Hz, 1 H) 7.81 (d, J=2.27 Hz, 2 H) 8.24 (s, 1 H) 8.43 (s, 1 H) 8.49 (d, J=4.55 Hz, 1 H) 8.64 (s, 1 H); HRMS (ESI+) calcd for C₂₃H₁₄ClF₄N₅ (MH+) 472.09466, found 472.0946.

### Example 231

### 4-[(3-chloro-4-fluorophenyl)amino]-6-[(pyridin-2-ylmethyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with pyridine-2-carbaldehyde (36.6 mg, 0.34 mmol) and NaCNBH₃ (11.6 mg, 0.18 mmol) in 4 mL EtOH. The crude product was purified by preparative HPLC, and lyophilized to give the product as a yellow solid (43.1 mg, 35%): 1H NMR (400 MHz, DMSO-D6) δ ppm 4.57 (d, J=6.06 Hz, 2 H) 7.22-7.32 (m, 3 H) 7.36-7.48 (m, 3 H) 7.52 (dd, J=6.57, 2.53 Hz, 1 H) 7.73-7.81 (m, 1 H) 7.90 (d, J=2.27 Hz, 1 H) 8.43 (s, 1 H) 8.55 (dd, J=4.42, 1.39 Hz, 1 H) 9.53 (s, 1 H); HRMS (ESI+) calcd for C₂₃H₁₄ClF₄N₅ (MH+) 472.09466, found 472.0948.

### Example 232

### 4-[(3-chloro-4-fluorophenyl)amino]-6-[(3-cyanobenzyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 3-formyl-benzonitrile (44.9 mg, 0.34 mmol) and NaCNBH₃ (11.6 mg, 0.18 mmol) in 4 mL EtOH. The crude product was purified by preparative HPLC, and lyophilized to give the product as a yellow solid (53.7 mg, 41%): 1H NMR (400 MHz, DMSO-D6) δ ppm 4.50 (d, J=5.81 Hz, 2 H) 7.20-7.31 (m, 2 H) 7.38 (d, J=2.27 Hz, 1 H) 7.43 (t, J=8.97 Hz, 1 H) 7.52 (dd, J=6.57, 2.53 Hz, 1 H) 7.57 (t, J=7.83 Hz, 1 H) 7.70-7.77 (m, 2 H) 7.81 (d, J=2.27 Hz, 1 H) 7.86 (s, 1 H) 8.44 (s, 1 H) 9.50 (s, 1 H); HRMS (ESI+) calcd for C₂₅H₁₄ClF₄N₅ (MH+) 496.09466, found 496.0943.

### Example 233

### 4-[(3-chloro-4-fluorophenyl)amino]-6-[(2-cyanobenzyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 2-formyl-benzonitrile (67.9 mg, 0.52 mmol) and NaCNBH₃ (21.6 mg, 0.34 mmol) in 4 mL EtOH. The crude product was purified by preparative HPLC, and lyophilized to give the product as a yellow solid (37.9 mg, 29%): 1H NMR (400 MHz, DMSO-D6) δ ppm 4.62 (d, J=5.56 Hz, 2 H) 7.22 (m, 1 H) 7.24-7.31 (m, 1 H) 7.39-7.44(m, 2H) 7.48-7.55 (m, 2 H) 7.59 (d, J=8.08 Hz, 1 H) 7.66-7.73 (m, 1 H) 7.82-7.91 (m, 2 H) 8.46 (s, 1 H) 9.52 (s, 1 H); HRMS (ESI+) calcd for C₂₅H₁₄ClF₄N₅ (MH+) 496.09466, found 496.0943.

### Example 234

### 4-[(3-chloro-4-fluorophenyl)amino]-6-[(4-cyanobenzyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 4-formyl-benzonitrile (44.9 mg, 0.34 mmol) and NaCNBH₃ (11.6 mg, 0.18 mmol) in 4 mL EtOH. The crude product was purified by preparative HPLC, and lyophilized to give the product as a yellow solid (69.7 mg, 53%): 1H NMR (400 MHz, DMSO-D6) δ ppm 4.55 (d, J=5.81 Hz, 2 H) 7.23-7.28 (m, 1 H) 7.30 (t, J=6.06 Hz, 1 H) 7.35 (d, J=2.02 Hz, 1 H) 7.42 (t, J=8.97 Hz, 1 H) 7.50 (dd, J=6.57, 2.53 Hz, 1 H) 7.56 (d, J=8.34 Hz, 2 H) 7.75-7.86 (m, 3 H) 8.44 (s, 1 H) 9.47 (s, 1 H);HRMS (ESI+) calcd for C₂₅H₁₄ClF₄N₅ (MH+) 496.09466, found 496.0942.

### Example 235

### 4-[(3-chloro-4-fluorophenyl)amino]-6-[(2-fluorobenzyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 2-fluoro-benzaldehyde (42.5 mg, 0.34 mmol) and NaCNBH₃ (11.6 mg, 0.18 mmol) in 4 mL EtOH. The crude product was purified by preparative HPLC, and lyophilized to give the product as a yellow solid (22.5 mg, 18%): 1H NMR (400 MHz, DMSO-D6) δ ppm 4.48 (d, J=5.56 Hz, 2 H) 7.10 (t, J=5.68 Hz, 1 H) 7.16-7.26 (m, 2 H) 7.25-7.31 (m, 1 H) 7.32-7.39 (m, 1 H) 7.40-7.48 (m, 3 H) 7.54 (dd, J=6.57, 2.53 Hz, 1 H) 7.84 (d, J=2.27 Hz, 1 H) 8.44 (s, 1 H) 9.53 (s, 1 H); HRMS (ESI+) calcd for C₂₄H₁₄ClF₅N₄ (MH+) 489.08999, found 489.0897.

### Example 236

### 4-[(3-chloro-4-fluorophenyl)amino]-6-{[(1-methyl-1H-imidazol-2-yl)methyl]amino}-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 1-methyl-1H-imidazole-2-carbaldehyde (37.7 mg, 0.34 mmol) and NaCNBH₃ (11.6 mg, 0.18 mmol) in 4 mL EtOH. The crude product was purified by preparative HPLC, and lyophilized to give the product as a yellow solid (54.6 mg, 44%): 1H NMR (400 MHz, DMSO-D6) δ ppm 3.65 (s, 3 H) 4.43 (d, J=5.05 Hz, 2 H) 6.83 (d, J=1.26 Hz, 1 H) 7.01 (t, J=5.05 Hz, 1 H) 7.15 (d, J=1.26 Hz, 1 H) 7.29-7.36 (m, 1 H) 7.46 (t, J=8.97 Hz, 1 H) 7.52 (d, J=2.27 Hz, 1 H) 7.57 (dd, J=6.57, 2.78 Hz, 1 H) 7.89 (d, J=2.27 Hz, 1 H) 8.44 (s, 1 H) 9.57 (s, 1 H); HRMS (ESI+) calcd for C₂₂H₁₅ClF₄N₆ (MH+) 475.10556, found 475.106.

### Example 237

### 4-[(3-chloro-4-fluorophenyl)amino]-6-({[1-(phenylsulfonyl)-1H-pyrrol-2-yl]methyl}amino)-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 1-benzenesulfonyl-1H-pyrrole-2-carbaldehyde (80.5 mg, 0.34 mmol) and NaCNBH₃ (11.6 mg, 0.18 mmol) in 4 mL EtOH. The crude product was purified by preparative HPLC, and lyophilized to give the product as a yellow solid (87.1 mg, 55%): 1H NMR (400 MHz, DMSO-D6) δ ppm 4.56 (d, J=5.56 Hz, 2 H) 6.24-6.42 (m, 2 H) 6.80 (t, J=5.56 Hz, 1 H) 7.24-7.36 (m, 1 H) 7.39-7.50 (m, 3 H) 7.52-7.60 (m, 3 H) 7.64 (d, J=2.02 Hz, 1 H) 7.69 (t, J=7.45 Hz, 1 H) 7.86-7.94 (m, 2 H) 8.37-8.50 (m, 1 H) 9.59 (s, 1 H); HRMS (ESI+) calcd for C₂₈H₁₈ClF₄N₅O₂S (MH+) 600.08786, found 600.0885.

### Example 238

### 4-[(3-chloro-4-fluorophenyl)amino]-6-{[(4,5-dimethyl-2-furyl)methyl]amino}-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 4,5-dimethyl-furan-2-carbaldehyde (42.5 mg, 0.34 mmol) and NaCNBH₃ (11.6 mg, 0.18 mmol) in 4 mL EtOH. The crude product was purified by preparative HPLC, and lyophilized to give the product as a yellow solid (18.9 mg, 15%): 1H NMR (400 MHz, DMSO-D6) δ ppm 1.85 (s, 3 H) 2.14 (s, 3 H) 4.32 (d, J=5.56 Hz, 2 H) 6.14 (s, 1 H) 7.02 (t, J=5.56 Hz, 1 H) 7.27-7.36 (m, 1 H) 7.41-7.49 (m, 2 H) 7.55 (dd, J=6.69, 2.65 Hz, 1 H) 7.79 (d, J=2.27 Hz, 1 H) 8.42 (s, 1 H) 9.56 (s, 1 H); HRMS (ESI+) calcd for C₂₄H₁₇ClF₄N₄O (MH+) 489.10998, found 489.1107.

### Example 239

### 4-[(3-chloro-4-fluorophenyl)amino]-6-[(1,3-thiazol-5-ylmethyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with thiazole-5-carbaldehyde (38.7 mg, 0.34 mmol) and NaCNBH₃ (11.6 mg, 0.18 mmol) in 4 mL EtOH. The crude product was purified by preparative HPLC, and lyophilized to give the product as a yellow solid (43.9 mg, 35%): 1H NMR (400 MHz, DMSO-D6) δ ppm 4.72 (d, J=6.06 Hz, 2 H) 7.23 (t, J=5.81 Hz, 1 H) 7.29-7.38 (m, 1 H) 7.46 (t, J=9.09 Hz, 1 H) 7.54 (d, J=2.27 Hz, 1 H) 7.58 (dd, J=6.69, 2.65 Hz, 1 H) 7.77 (d, J=2.27 Hz, 1 H) 7.93 (s, 1 H) 8.44 (s, 1 H) 9.00 (s, 1 H) 9.55 (s, 1 H);HRMS (ESI+) calcd for C₂₁H₁₂ClF₄N₅S (MH+) 478.05108, found 478.051.

### Example 240

### 4-[(3-chloro-4-fluorophenyl)amino]-6-[(pyrimidin-5-ylmethyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with pyrimidine-5-carbaldehyde (57.0 mg, 0.53 mmol) and NaCNBH₃ (21.6 mg, 0.34 mmol) in 4 mL EtOH. The crude product was purified by preparative HPLC, and lyophilized to give the product as a yellow solid (68.2 mg, 55%): 1H NMR (400 MHz, DMSO-D6) δ ppm 4.50 (d, J=5.56 Hz, 2 H) 7.22 (t, J=11.12 Hz, 1 H) 7.27-7.35 (m, 1 H) 7.40-7.51 (m, 2 H) 7.56 (dd, J=6.57, 2.53 Hz, 1 H) 7.79 (d, J=2.02 Hz, 1 H) 8.44 (s, 1 H) 8.86 (s, 2 H) 9.12 (s, 1 H) 9.57 (br, s, 1 H); HRMS (ESI+) calcd for C₂₂H₁₃ClF₄N₆ (MH+) 473.08991, found 473.0896.

### Example 241

### 4-[(3-chloro-4-fluorophenyl)amino]-6-{[(4,6-dimethoxypyrimidin-5-yl)methyl]amino}-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 2,4-dimethoxy-pyrimidine-5-carbaldehyde (57.5 mg, 0.34 mmol) and NaCNBH₃ (11.6 mg, 0.18 mmol) in 4 mL EtOH. The crude product was purified by preparative HPLC, and lyophilized to give the product as a yellow solid (44.6 mg, 32%): 1H NMR (400 MHz, DMSO-D6) δ ppm 3.88 (s, 3 H) 3.95 (s, 3 H) 4.27 (d, J=5.31 Hz, 2 H) 6.93 (t, J=5.43 Hz, 1 H) 7.29-7.32 (m, 1 H) 7.36-7.50 (m, 2 H) 7.55 (dd, J=6.69, 2.65 Hz, 1 H) 7.78 (d, J=1.77 Hz, 1 H) 8.30 (s, 1 H) 8.44 (s, 1 H) 9.55 (s, 1 H); HRMS (ESI+) calcd for C₂₄H₁₇ClF₄N₆O₂ (MH+) 533.11104, found 533.1112.

### Example 242

### 4-[(3-chloro-4-fluorophenyl)amino]-6-({[2-(phenylsulfonyl)-1,3-thiazol-5-yl]methyl}amino)-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 2-benzenesulfonyl-thiazole-5-carbaldehyde (86.6 mg, 0.34 mmol) and NaCNBH₃ (11.6 mg, 0.18 mmol) in 4 mL EtOH. The crude product was purified by preparative HPLC, and lyophilized to give the product as a yellow solid (19.0 mg, 12%): 1H NMR (400 MHz, DMSO-D6) δ ppm 4.79 (d, J=5.81 Hz, 2 H) 7.28-7.39 (m, 2 H) 7.46 (t, J=8.97 Hz, 1 H) 7.54 (s, 1 H) 7.56-7.63 (m, 1 H) 7.64-7.72 (m, J=7.58 Hz, 2 H) 7.72-7.85 (m, 2 H) 8.01 (d, J=7.58 Hz, 2 H) 8.11 (s, 1 H) 8.45 (s, 1 H) 9.52 (s, 1 H); HRMS (ESI+) calcd for C₂₇H₁₆ClF₄N₅O₂(MH+) 618.04428, found 618.0426.

### Example 243

### 4-[(3-chloro-4-fluorophenyl)amino]-6-{[(2-morpholin-4-yl-1,3-thiazol-5-yl)methyl]amino}quinoline-3-carbonitrile

Following the procedure described above in Example 4, 6-amino-4-(3-chloro-4-fluoro-phenylamino)-quinoline-3-carbonitrile (0.10 g, 0.32 mmol) was reacted with 2-morpholin-4-yl-thiazole-5-carbaldehyde (82.4 mg, 0.42 mmol) and NaCNBH₃ (14.1 mg, 0.22 mmol) in 4 mL EtOH. The crude product was purified by preparative HPLC, and lyophilized to give the product as a yellow solid (66.5 mg, 42%): 1H NMR (400 MHz, DMSO-D6) δ ppm 3.26-3.31 (m, 4 H) 3.63-3.70 (m, 4 H) 4.43 (d, J=5.81 Hz, 2 H) 6.77 (t, J=5.94 Hz, 1 H) 7.17 (s, 1 H) 7.20-7.27 (m, 2 H) 7.31 (dd, J=9.09, 2.53 Hz, 1 H) 7.39-7.49 (m, 2 H) 7.70 (d, J=8.84 Hz, 1 H) 8.33 (s, 1 H) 9.32 (s, 1 H); HRMS (ESI+) calcd for C₂₄H₂₀ClFN₆OS (MH+) 495.11646, found 495.1153.

### Example 244

### 4-[(3-chloro-4-fluorophenyl)amino]-6-{[(2-morpholin-4-yl-1,3-thiazol-5-yl)methyl]amino}-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 2-morpholin-4-yl-thiazole-5-carbaldehyde (67.8 mg, 0.34 mmol) and NaCNBH₃ (11.6 mg, 0.18 mmol) in 4 mL EtOH. The crude product was purified by preparative HPLC, and lyophilized to give the product as a yellow solid (58.1 mg, 39%): 1H NMR (400 MHz, DMSO-D6) δ ppm 3.20-3.36 (m, 4 H) 3.60-3.75 (m, 4 H) 4.48 (d, J=5.56 Hz, 2 H) 7.08 (t, 1 H) 7.21 (s, 1 H) 7.29-7.38 (m, 1 H) 7.42-7.51 (m, 2 H) 7.57 (dd, J=6.57, 2.53 Hz, 1 H) 7.75 (d, J=2.02 Hz, 1 H) 8.44 (s, 1 H) 9.54 (s, 1 H); HRMS (ESI+) calcd for C₂₅H₁₉ClF₄N₆OS (MH+) 563.10384, found 563.1025.

### Example 245

### 4-[(3-chloro-4-fluorophenyl)amino]-6-({[2-(phenylsulfonyl)-1,3-thiazol-5-yl]methyl}amino)quinoline-3-carbonitrile

Following the procedure described above in Example 4, 6-amino-4-(3-chloro-4-fluoro-phenylamino)-quinoline-3-carbonitrile (0.10 g, 0.32 mmol) was reacted with 2-benzenesulfonyl-thiazole-5-carbaldehyde (71.3 mg, 0.28 mmol) and NaCNBH₃ (11.3 mg, 0.18 mmol) in 4 mL EtOH. The crude product was purified by preparative HPLC, and lyophilized to give the product as a yellow solid (11.0 mg, 8%): 1H NMR (400 MHz, DMSO-D6) δ ppm 4.79 (d, J=6.06 Hz, 2 H) 7.10 (t, J=6.19 Hz, 1 H) 7.27-7.41 (m, 3 H) 7.49 (t, J=8.97 Hz, 1 H) 7.57 (dd, J6.57, 2.78 Hz, 1 H) 7.70-7.89 (m, 4 H) 8.06 (dd, J=8.46, 1.14 Hz, 2 H) 8.15 (s, 1 H) 8.40(s, 1 H) 9.38 (s, 1 H); HRMS (ESI+) calcd for C₂₆H₁₇ClFN₅O₂S₂ (MH+) 550.05690, found 550.0575.

### Example 246

### 4-(cycloheptylamino)-6-[(pyridin-3-ylmethyl)amino]quinoline-3-carbonitrile

Step 1: In a 100 mL round-bottomed flask equipped with a condenser, 6-nitro-4-oxo-1,4-dihydro-quinoline-3-carbonitrile (3.3 g, 15.3 mmol) was taken up in 40 mL POCl₃ and heated at reflux for 6 hours. The reaction mixture was then stirred at RT overnight, and then POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum overnight to give a light brown solid as 4-chloro-6-nitro-quinoline-3-carbonitrile (3.2 g, 90% yield).

Step 2: In a microwave vial, the product from step 1 (0.4 g, 1.71 mmol) was taken up in 2 mL EtOH and cycloheptyl amine (0.23 g, 2.05 mmol) was added. The vial was crimp-sealed and heated in a microwave reactor at 150° C. for 45 minutes. This was repeated with a second batch of reagents, with 0.6 g of 4-chloro-6-nitro-quinoline-3-carbonitrile. The contents of the two vials were combined and evaporated down the solvent to a yellow residue. The residue was partitioned between ether and H₂O, the resulting suspension was filtered, washed with H₂O, dried under high vacuum overnight to give a yellow solid as 4-cycloheptylamino-6-nitro-quinoline-3-carbonitrile (0.93 g, 70% yield).

Step 3: In a microwave vial, the product from step 2 (0.30 g, 0.97 mmol) was taken up in 2 mL EtOH and tin chloride dihydrate (1.09 g, 4.83 mmol) was added. The vial was sealed and heated in a microwave reactor at 110° C. for 10 minutes, until LC/MS analysis showed complete disappearance of the nitroquinoline. This was repeated with a second batch of reagents, with 0.63 g of 4-cycloheptylamino-6-nitro-quinoline-3-carbonitrile. The contents of the two vials were combined and then poured into ice water, and the reaction worked up as described above in Example 229 for the synthesis of 6-amino-4-[(3-chloro-4-fluorophenyl)amino]-8-(trifluoromethyl)quinoline-3-carbonitrile. 6-amino-4-cycloheptylamino-quinoline-3-carbonitrile was obtained as a yellow solid (0.70 g, 83% yield).

Step 4: Following the procedure described above in Example 4, 6-amino-4-cycloheptylamino-quinoline-3-carbonitrile (80.0 mg, 0.29 mmol) was reacted with pyridine-3-carbaldehyde (39.7 mg, 0.37 mmol) and NaCNBH₃ (12.5 mg, 0.20 mmol) in 3 mL EtOH. The crude product was purified by preparative HPLC, and lyophilized to give the product as a yellow solid (73.6 mg, 70%): 1H NMR (400 MHz, DMSO-D6) δ ppm 1.37-1.78 (m, 10 H) 1.89-2.05 (m, 2 H) 4.31-4.39 (m, 1 H) 4.41 (d, J=5.81 Hz, 2 H) 6.65 (t, J=5.94 Hz, 1 H) 6.88 (d, J=8.34 Hz, 1 H) 7.05 (d, J=2.27 Hz, 1 H) 7.15 (dd, J=8.97, 2.40 Hz, 1 H) 7.29 (dd, J=7.33, 4.29 Hz, 1 H) 7.49 (d, J=9.09 Hz, 1 H) 7.71-7.80 (m, 1 H) 8.10 (d, J=6.32 Hz, 1 H) 8.39 (dd, J=4.80, 1.77 Hz, 1 H) 8.60 (d, J=1.77 Hz, 1 H); HRMS (ESI+) calcd for C₂₃H₂₅N₅ (MH+) 372.21827, found 372.2186.

### Example 247

### 6-[(3-Cyanobenzyl)amino]-4-(cycloheptylamino)quinoline-3-carbonitrile

Following the procedure described above in Example 4, 6-amino-4-cycloheptylamino-quinoline-3-carbonitrile (80.0 mg, 0.29 mmol) was reacted with 3-formyl-benzonitrile (48.7 mg, 0.37 mmol) and NaCNBH₃ (12.5 mg, 0.20 mmol) in 3 mL EtOH. The crude product was purified by preparative HPLC, and lyophilized to give the product as a yellow solid (43.6 mg, 39%): 1H NMR (400 MHz, DMSO-D6) δ ppm 1.51-1.93 (m, 10 H) 1.96-2.17 (m, 2 H) 4.35-4.55 (m, J=9.09 Hz, 1 H) 4.60 (d, J=5.81 Hz, 2 H) 6.88-7.01 (m, 2 H) 7.12 (d, J=2.27 Hz, 1 H) 7.30 (dd, J=8.84, 2.27 Hz, 1 H) 7.58-7.69 (m, 2 H) 7.83 (dd, J=20.34, 7.71 Hz, 2 H) 8.01 (s, 1 H) 8.21-8.30 (m, 1 H); HRMS (ESI+) calcd for C₂₅H₂₅N₅ (MH+) 396.21827, found 396.218.

### Example 248

### 4-(cycloheptylamino)-6-{[3-(methylsulfonyl)benzyl]amino}quinoline-3-carbonitrile

Following the procedure described above in Example 4, 6-amino-4-cycloheptylamino-quinoline-3-carbonitrile (80.0 mg, 0.29 mmol) was reacted with 3-methanesulfonyl-benzaldehyde (68.3 mg, 0.37 mmol) and NaCNBH₃ (12.5 mg, 0.20 mmol) in 3mL EtOH. The crude product was purified by preparative HPLC, and lyophilized to give the product as a yellow solid (58.2 mg, 46%): 1H NMR (400 MHz, DMSO-D6) δ ppm 1.34-1.87 (m, 10 H) 1.90-2.07 (m, 2 H) 3.12 (s, 3 H) 4.53 (s, 3 H) 7.20-7.26 (m, J=2.27 Hz, 1 H) 7.28(br, s, 1 H) 7.34 (dd, J=9.09, 2.27 Hz, 1 H) 7.52-7.64 (m, 2 H) 7.74 (dd, J=24.63, 7.71 Hz, 2 H) 7.94 (s, 1 H) 8.31 (d, J=7.58 Hz, 1 H) 8.69 (s, 1 H); HRMS (ESI+) calcd for C₂₅H₂₈N₄O₂S (MH+) 449.20057, found 449.2007.

### Example 249

### 4-(cycloheptylamino)-6-{[(1-methyl-1H-imidazol-2-yl)methyl]amino}quinoline-3-carbonitrile

Following the procedure described above in Example 4, 6-amino-4-cycloheptylamino-quinoline-3-carbonitrile (80.0 mg, 0.29 mmol) was reacted with 1-methyl-1H-imidazole-2-carbaldehyde (40.9 mg, 0.37 mmol) and NaCNBH₃ (12.5 mg, 0.20 mmol) in 3 mL EtOH. The crude product was purified by preparative HPLC, and lyophilized to give the product as a yellow solid (62.0 mg, 58%): 1H NMR (400 MHz, DMSO-D6) δ ppm 1.39-1.80 (m, 10 H) 1.93-2.11 (m, 2 H) 3.59 (s, 3 H) 4.27-4.52 (m, J=5.31 Hz, 3 H) 6.48 (t, J=5.31 Hz, 1 H) 6.71 (d, J=8.59 Hz, 1 H) 6.76 (d, J=1.01 Hz, 1 H) 7.00-7.11 (m, J=1.26 Hz, 1 H) 7.16-7.23 (m, 2 H) 7.50 (d, J=9.60 Hz, 1 H) 8.11 (s, 1 H); HRMS (ESI+) calcd for C₂₂H₂₆N₆ (MH+) 375.22917, found 375.2298.

### Example 250

### 4-({[3-cyano-4-(cycloheptylamino)quinolin-6-yl]amino}methyl)benzenesulfonamide

Following the procedure described above in Example 4, 6-amino-4-cycloheptylamino-quinoline-3-carbonitrile (70.0 mg, 0.25 mmol) was reacted with 4-formyl-benzenesulfonamide (60.2 mg, 0.33 mmol) and NaCNBH₃ (11.0 mg, 0.18 mmol) in 4 mL EtOH. The crude product was purified by preparative HPLC, and lyophilized to give the product as a yellow solid (72.1 mg, 64%): 1H NMR (400 MHz, DMSO-D6) δ ppm 1.34-1.87 (m, 10 H) 1.91-2.05 (m, J=18.19 Hz, 2 H) 4.39-4.63 (m, 3 H) 7.18-7.24 (m, J=1.77 Hz, 1 H) 7.26 (s, 2 H) 7.32 (dd, J=8.97, 2.15 Hz, 1 H) 7.55 (dd, J=16.30, 8.72 Hz, 3 H) 7.69-7.75 (m, 2 H) 8.26(br, s, 1 H) 8.68 (s, 1 H); HRMS (ESI+) calcd for C₂₄H₂₇N₅O₂S (MH+) 450.19582, found 450.1956.

### Example 251

### 4-(cycloheptylamino)-6-[(1H-pyrazol-5-ylmethyl)amino]quinoline-3-carbonitrile

Following the procedure described above in Example 4, 6-amino-4-cycloheptylamino-quinoline-3-carbonitrile (50.0 mg, 0.18 mmol) was reacted with 2H-pyrazole-3-carbaldehyde (26.0 mg, 0.27 mmol) and NaCNBH₃ (7.9 mg, 0.13 mmol) in 3 mL EtOH. The crude product was purified by preparative HPLC, and lyophilized to give the product as a yellow solid (26.9 mg, 42%): 1H NMR (400 MHz, DMSO-D6) δ ppm 1.37-1.78 (m, 10 H) 1.95 (d, J=13.90 Hz, 2 H) 4.31 (d, J=5.31 Hz, 2 H) 4.34-4.42 (m, 1 H) 6.18 (d, J=2.27 Hz, 2 H) 6.29 (t, J=5.43 Hz, 1 H) 6.90 (d, J=8.84 Hz, 1 H) 7.07 (d, J=2.27 Hz, 1 H) 7.16 (dd, J=8.84, 2.27 Hz, 1 H) 7.45 (d, J=8.84 Hz, 1 H) 7.51 (d, J=1.52 Hz, 1 H) 8.04-8.12 (m, 1 H); HRMS (ESI+) calcd for C₂₁H₂₄N₆ (MH+) 361.21352, found 361.2141.

### Example 252

### 4-(cycloheptylamino)-6-[(2-morpholin-4-ylethyl)amino]quinoline-3-carbonitrile

Following the procedure described above in Example 4, 6-amino-4-cycloheptylamino-quinoline-3-carbonitrile (70.0 mg, 0.25 mmol) was reacted with NaCNBH₃ (11.0 mg, 0.18 mmol) and morpholin-4-yl-acetaldehyde (prepared by heating the corresponding dimethyl acetal (70.0 mg, 0.40 mmol) in 1.2 mL concentrated HCl for 5 minutes in a microwave reactor at 110° C., then neutralizing with solid NaHCO₃ until pH=6) in 4 mL EtOH. The crude product was purified by preparative HPLC, and lyophilized to give the product as a yellow solid (27.6 mg, 28%): 1H NMR (400 MHz, DMSO-D6) δ ppm 0.92 (t, J=7.07 Hz, 2 H) 1.40-1.79 (m, 10 H) 1.91-2.05 (m, 2 H) 2.39 (s, 2 H) 2.46-2.58 (m, 4 H) 3.18-3.30 (m, 2 H) 4.28-4.50 (m, 2 H) 5.81 (s, 1 H) 6.89-7.06 (m, 2 H) 7.14 (dd, J=8.97, 2.15 Hz, 1 H) 7.47 (d, J=8.84 Hz, 1 H) 8.08 (s, 1 H) 8.25 (s, 1 H); HRMS (ESI+) calcd for C₂₃H₃₁N₅O (MH+) 394.26014, found 394.26.

### Example 253

### 4-(cycloheptylamino)-6-{[(1-oxidopyridin-2-yl)methyl]amino}quinoline-3-carbonitrile

Following the procedure described above in Example 4, 6-amino-4-cycloheptylamino-quinoline-3-carbonitrile (80.0 mg, 0.29 mmol) was reacted with 1-oxy-pyridine-2-carbaldehyde (45.7 mg, 0.37 mmol) and NaCNBH₃ (12.5 mg, 0.20 mmol) in 3 mL EtOH. The crude product was purified by preparative HPLC, and lyophilized to give the product as a yellow solid (63.3 mg, 57%): 1H NMR (400 MHz, DMSO-D6) δ ppm 1.40-1.86 (m, 10 H) 1.92-2.06 (m, 2 H) 4.45-4.71 (m, 3 H) 7.21-7.43 (m, 4 H) 7.59 (dd, J=20.46, 9.09 Hz, 1 H) 8.26-8.42 (m, 2 H) 8.52 (d, J=8.59 Hz, 1 H) 8.71 (s, 1 H); HRMS (ESI+) calcd for C₂₃H₂₅N₅O (MH+) 388.21319, found 388.2134.

### Example 254

### 4-[(3-chloro-4-fluorophenyl)amino]-6-[(1H-imidazol-5-ylmethyl)amino]-2-methylquinoline-3-carbonitrile

Step 1: In a 500 mL round-bottomed flask equipped with a condenser, cyano-acetic acid methyl ester (15.7 g, 158.2 mmol) and triethyl orthoacetate (25.7 g, 158.2 mmol) were taken up in 200 mL acetic anhydride and heated to 90° C. for 7.5 hours under nitrogen. The reaction mixture was then stirred at RT overnight, and then the solvent was removed under reduced pressure. 10 mL Ether and 20 mL hexane were added to the dark yellow liquid residue, a crystal of the product was also added to this two-layer solution. The solution was put into the refrigerator. After sitting in the refrigerator overnight, lots of crystals formed, the mixture was filtered, washed with hexane first, then washed with small amount of ether, dried under vacuum overnight to give a white crystal solid as 2-cyano-3-ethoxy-but-2-enoic acid methyl ester (7.1 g, 27% yield): 1H NMR (400 MHz, CHLOROFORM-D) δ ppm 1.44 (t, J=7.07 Hz, 3 H) 2.62 (s, 3 H) 3.78 (s, 3 H) 4.29 (q, J=7.07 Hz, 2 H).

Step 2: The procedure described above in Example 229 was followed, reacting 4-nitro-phenylamine (5.53 g, 40.0 mmol) with the product from the previous step (7.1 g, 42.0 mmol) and Cs₂CO₃ (26.1 mg, 80.0 mmol) in 25 mL DMF. An orange solid was obtained as product methyl-2-cyano-3-[(4-nitrophenyl)amino]but-2-enoate (WAY-199403, 7.8 g, 74%): 1H NMR (400 MHz, DMSO-D6) δ ppm 2.64 (s, 3 H) 3.89 (s, 3 H) 7.75 (d, J=8.34 Hz, 2 H) 8.42 (d, J=8.84 Hz, 2 H) 11.64 (s, 1 H); HRMS (ESI+) calcd for C₁₂H₁₁N₃O₄ (MH+) 262.08223, found 262.08234.

Step 3: Following the procedure described above in Example 229, the product from step 2 (6.23 g, 23.9 mmol) was taken up in 240 mL dowtherm A and heated at reflux for 4 hours under argon. Part of the crude product (1.5 g) was dissolved in 8 mL DMSO and heated to 80° C. for 5 minutes, then filtered, washed with small amount of DMSO. To the filtrate was added H₂O (150 mL), precipitate formed. The mixture was filtered, washed with H₂O, dried under vacuum to give 2-methyl-6-nitro-4-oxo-1,4-dihydroquinoline-3-carbonitrile (0.4 g, 21%) as a light brown solid: 1H NMR (400 MHz, DMSO-D6) δ ppm 2.57 (s, 3 H) 7.72 (d, J=9.09 Hz, 1 H) 8.46 (dd, J=9.09, 2.53 Hz, 1 H) 8.71 (d, J=2.78 Hz, 1 H) 12.97 (s, 1 H); HRMS (ESI+) calcd for C₁₁H₇N₃O₃ (MH+) 230.05602, found 230.0565.

Step 4: In a 100 mL round-bottomed flask equipped with a condenser, the product from step 3 (0.35 g, 1.5 mmol) was taken up in 10 mL POCl₃ and heated at reflux for 8 hours. The reaction mixture was then stirred at RT overnight, and then POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum overnight to give a black solid as 4-chloro-2-methyl-6-nitro-quinoline-3-carbonitrile (0.32 g, 85% yield).

Step 5: Following the procedure described above in Example 229, 4-chloro-2-methyl-6-nitro-quinoline-3-carbonitrile (0.32 g, 1.29 mmol) was reacted with 3-chloro-4-fluoroaniline (0.23 g, 1.55 mmol) in 4 mL EtOH. After work up, 4-(3-chloro-4-fluoro-phenylamino)-2-methyl-6-nitro-quinoline-3-carbonitrile was obtained as a dark brown solid (0.19 g, 40% yield).

Step 6: Following the procedure described above in Example 229, 4-(3-chloro-4-fluoro-phenylamino)-2-methyl-6-nitro-quinoline-3-carbonitrile (0.19 g, 0.52 mmol) was reacted with tin chloride dihydrate (0.59 g, 2.61 mmol) in 3 mL EtOH. After work up, 6-amino-4-(3-chloro-4-fluoro-phenylamino)-2-methyl-quinoline-3-carbonitrile was obtained as a brown solid (0.14 g, 81% yield).

Step 7: Following the procedure described above in Example 4, 6-amino-4-(3-chloro-4-fluoro-phenylamino)-2-methyl-quinoline-3-carbonitrile (80.0 mg, 0.25 mmol) was reacted with 4(5)-imidazolecarboxyaldehyde (30.6 mg, 0.32 mmol) and NaCNBH₃ (10.8 mg, 0.17 mmol) in 4 mL EtOH. The crude product was purified by preparative HPLC, and lyophilized to give the product as a yellow solid (29.0 mg, 29%): 1H NMR (400 MHz, DMSO-D6) δ ppm 2.48 (s, 3 H) 4.15 (d, J=5.31 Hz, 2 H) 6.32 (s, 1 H) 6.94 (s, 1 H) 7.01-7.15 (m, 2 H) 7.22-7.38 (m, 3 H) 7.47-7.60 (m, 2 H) 9.16 (s, 1 H); HRMS (ESI+) calcd for C₂₁H₁₆ClFN₆ (MH+) 407.11818, found 407.1178.

### Example 255

### 4-[(3-chloro-4-fluorophenyl)amino]-2-methyl-6-[(pyridin-3-ylmethyl)amino]quinoline-3-carbonitrile

Following the procedure described above in Example 4, 6-amino-4-(3-chloro-4-fluoro-phenylamino)-2-methyl-quinoline-3-carbonitrile (52.0 mg, 0.16 mmol) was reacted with pyridine-3-carbaldehyde (22.2 mg, 0.21 mmol) and NaCNBH₃ (7.0 mg, 0.11 mmol) in 3 mL EtOH. The crude product was purified by preparative HPLC, and lyophilized to give the product as a yellow solid (12.7 mg, 19%): 1H NMR (400 MHz, DMSO-D6) δ ppm 2.49 (s, 3 H) 4.30 (d, J=5.81 Hz, 2 H) 6.73 (t, J=5.94 Hz, 1 H) 6.92-7.09 (m, 2 H) 7.22-7.33 (m, 4 H) 7.58 (d, J=8.84 Hz, 1 H) 7.68 (dd, J=7.83, 1.77 Hz, 1 H) 8.39 (dd, J=4.80, 1.52 Hz, 1 H) 8.51 (d, J=1.52 Hz, 1 H) 9.13 (s, 1 H); HRMS (ESI+) calcd for C₂₃H₁₇ClFN₅ (MH+) 418.12293, found 418.1233.

### Example 256

### 4-[(3-chloro-4-fluorophenyl)amino]-6-[(2-pyridin-2-ylcyclopentyl)amino]quinoline-3-carbonitrile

Following the procedure described above in Example 4, 6-amino-4-(3-chloro-4-fluoro-phenylamino)-quinoline-3-carbonitrile (100 mg, 0.32 mmol) was reacted with 2-pyridin-2-yl-cyclopentanone (97.0 mg, 0.61 mmol) and NaCNBH₃ (21.1 mg, 0.33 mmol) in 5 mL EtOH. The crude product was purified by preparative HPLC, and lyophilized to give the product as a yellow solid (6.0 mg, 4%): 1H NMR (400 MHz, DMSO-D6) δ ppm 1.24-1.36 (m, 1 H) 1.61-1.82 (m, 3 H) 1.89-2.00 (m, 1 H) 2.13-2.21 (m, 1 H) 2.97-3.10 (m, 1 H) 3.92-4.04 (m, 1 H) 6.37 (d, J=7.83 Hz, 1 H) 6.74 (d, J=2.27 Hz, 1 H) 6.93-6.99 (m, 1 H) 7.01-7.07 (m, 2 H) 7.11 (d, J=7.83 Hz, 1 H) 7.22-7.30 (m, 2 H) 7.41-7.48 (m, 2 H) 8.11 (s, 1 H) 8.29 (d, J=3.79 Hz, 1 H) 9.08 (s, 1 H); HRMS (ESI+) calcd for C₂₆H₂₁ClFN₅ (MH+) 458.15423, found 458.1545.

### Example 257

### 4-[(3-chloro-4-fluorophenyl)amino]-6-{[(1-oxidopyridin-2-yl)methyl]amino}-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (80.0 mg, 0.21 mmol) was reacted with 1-oxy-pyridine-2-carbaldehyde (53.6 mg, 0.43 mmol) and NaCNBH₃ (18.4 mg, 0.29 mmol) in 4 mL EtOH. The crude product was purified by preparative HPLC, and lyophilized to give the product as a yellow solid (21.5 mg, 21%): 1H NMR (400 MHz, DMSO-D6) δ ppm 4.53 (d, J=6.32 Hz, 2 H) 7.11-7.28 (m, 6 H) 7.32 (t, J=8.97 Hz, 1 H) 7.44 (dd, J=6.57, 2.53 Hz, 1 H) 7.79 (d, J=2.27 Hz, 1 H) 8.23 (d, J=6.57 Hz, 1 H) 8.31 (s, 1 H) 9.45 (s, 1 H); HRMS (ESI+) calcd for C₂₃H₁₄ClF₄N₅O (MH+) 488.08957, found 488.0894.

### Example 258

### 6-{[(6-bromopyridin-2-yl)methyl]amino}-4-[(3-chloro-4fluorophenyl)amino]quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 6-bromo-pyridine-2-carbaldehyde (71.4 mg, 0.38 mmol) and NaCNBH₃ (11.4 mg, 0.18 mmol) in 4 mL EtOH. The crude product was purified by preparative HPLC, and lyophilized to give the product as a yellow solid (62.0 mg, 49%): 1H NMR (400 MHz, DMSO-D6) δ ppm 4.68 (d, J=6.06 Hz, 2 H) 7.17 (t, J=6.32 Hz, 1 H) 7.28 (d, J=2.27 Hz, 1 H) 7.30-7.39 (m, 1 H) 7.47-7.60 (m, 4 H) 7.66 (d, J=7.83 Hz, 1 H) 7.80-7.93 (m, 2 H) 8.48 (s, 1 H) 9.44 (s, 1 H); HRMS (ESI+) calcd for C₂₂H₁₄BrClFN₅ (MH+) 482.01779, found 482.0181.

### Example 259

### 6-{[(6-Bromopyridin-2-yl)methyl]amino}-4-[(3-chloro-4-fluorophenyl)amino]-8-(trifluoromethyl)quinoline-3-carbonitrile

Following the procedure described above in Example 4, 6-amino-4-[(3-chloro-4-fluorophenyl)amino]-8-(trifluoromethyl)quinoline-3-carbonitrile (0.10 g, 0.26 mmol) was reacted with 6-bromo-pyridine-2-carbaldehyde (73.3 mg, 0.39 mmol) and NaCNBH₃ (11.6 mg, 0.18 mmol) in 4 mL EtOH. The crude product was purified by preparative HPLC, and lyophilized to give the product as a yellow solid (68.2 mg, 47%): 1H NMR (400 MHz, DMSO-D6) δ ppm 4.56 (d, J=6.06 Hz, 2 H) 7.24-7.33 (m, 2 H) 7.37-7.44 (m, 3 H) 7.48-7.54 (m, 2 H) 7.70 (t, J=7.71 Hz, 1 H) 7.86 (d, J=2.27 Hz, 1 H) 8.41 (s, 1 H) 9.45-9.52 (m, 1 H); HRMS (ESI+) calcd for C₂₃H₁₃BrClF₄N₅ (MH+) 550.00517, found 550.0054.

### Example 260

### 4-[(3-chloro-4-fluorophenyl)amino]-6-[(1-pyrazin-2-ylethyl)amino]quinoline-3-carbonitrile

Following the procedure described above in Example 4, 6-amino-4-(3-chloro-4-fluoro-phenylamino)-quinoline-3-carbonitrile (80 mg, 0.26 mmol) was reacted with 1-pyrazin-2-yl-ethanone (213.3 mg, 1.98 mmol) and NaCNBH₃ (22.8 mg, 0.36 mmol) in 4 mL EtOH. The crude product was purified by preparative HPLC under basic condition, and lyophilized to give a yellow solid as Et₃N salt form (8.0 mg, 6%): 1H NMR (400 MHz, DMSO-D6) δ ppm 1.48 (d, J=6.82 Hz, 3 H) 4.77-4.87 (m, 1 H) 6.87 (d, J=8.59 Hz, 1 H) 6.98 (d, J=2.27 Hz, 1 H) 7.03-7.10 (m, 1 H) 7.26-7.36 (m, 3 H) 7.64 (d, J=9.35 Hz, 1 H) 8.26 (s, 1 H) 8.43 (d, J=2.53 Hz, 1 H) 8.51 (dd, J=2.53, 1.52 Hz, 1 H) 8.61 (d, J=1.52 Hz, 1 H) 9.20 (s, 1 H); HRMS (ESI+) alcd for C₂₂H₁₆ClFN₆ (MH+) 419.11818, found 419.1176.

### Example 261

### 4-[(3-tert-butyl-1-methyl-1H-pyrazol-5-yl)amino]-6-[(1 H-imidazol-5-ylmethyl)amino]quinoline-3-carbonitrile

Step 1: In a microwave vial, 4-chloro-6-nitro-quinoline-3-carbonitrile (0.3 g, 1.28 mmol) and 5-tert-butyl-2-methyl-2H-pyrazol-3-ylamine (0.37 g, 2.43 mmol) were taken up in 4 mL DME. The vial was crimp-sealed and heated in a microwave reactor at 140° C. for 30 minutes. The content of the vial was evaporated down the solvent and the residue was partitioned between ether and saturated NaHCO₃ until pH=7, and stirred for 15 minutes, then evaporated some ether by rotovamp until precipitate formed. The mixture was filtered and dried under high vacuum overnight to give 4-[(3-Tert-butyl-1-methyl-1H-pyrazol-5-yl)amino]-6-nitroquinoline-3-carbonitrile as a yellow solid (0.33 g, 74% yield): 1H NMR (400 MHz, DMSO-D6) δ ppm 1.08 (s, 9 H) 3.16 (s, 3 H) 6.07 (s, 1 H) 7.96 (d, J=8.59 Hz, 1 H) 8.40 (d, J=7.33 Hz, 1 H) 8.61 (s, 1 H) 9.40 (s, 1 H) 10.17 (s, 1 H); HRMS (ESI+) calcd for C₁₈H₁₈N₆O₂ (MH+) 351.15640, found 351.1563.

Step 2: In a microwave vial, the product from the previous step (50 mg, 0.14 mmol) was taken up in 1.5 mL EtOH and tin chloride dihydrate (161.0 mg, 0.71 mmol) was added. The vial was sealed and heated in a microwave reactor at 110° C. for 10 minutes, until LC/MS analysis showed complete disappearance of the nitroquinoline. This was repeated with a second batch of reagents, with 0.25g of 4-[(3-tert-butyl-1-methyl-1H-pyrazol-5-yl)amino]-6-nitroquinoline-3-carbonitrile. The contents of the two vials were combined and then poured into ice water, and the reaction worked up as described above in Example 229. A yellow solid was obtained as product 6-amino-4-[(3-tert-butyl-1-methyl-1H-pyrazol-5-yl)amino]quinoline-3-carbonitrile (109.0 mg, 40% yield): 1H NMR (400 MHz, DMSO-D6) δ ppm 1.18-1.35 (m, 9 H) 3.62 (s, 3 H) 5.75 (s, 2 H) 6.13 (s, 1 H) 7.21-7.36 (m, 2 H) 7.69 (d, J=9.35 Hz, 1 H) 8.25 (s, 1 H) 9.19 (s, 1 H); HRMS (ESI+) calcd for C₁₈H₂₀N₆ (MH+) 321.18222, found 321.182.

Step 3: Following the procedure described above in Example 4, 6-amino-4-[(3-tert-butyl-1-methyl-1H-pyrazol-5-yl)amino]quinoline-3-carbonitrile (80 mg, 0.25 mmol) was reacted with 4(5)-imidazolecarboxyaldehyde (31.2 mg, 0.32 mmol) and NaCNBH₃ (11.0 mg, 0.18 mmol) in 5 mL EtOH. The crude product was purified by preparative HPLC, and lyophilized to give the product as a yellow solid (31.6 mg, 32%): 1H NMR (400 MHz, DMSO-D6) δ ppm 1.30 (s, 9 H) 3.65 (s, 3 H) 4.32 (s, 2 H) 6.22 (s, 1 H) 6.55 (s, 1 H) 7.16 (s, 1 H) 7.29 (s, 1 H) 7.43 (d, J=8.34 Hz, 1 H) 7.61-7.76 (m, 2 H) 8.29 (s, 1 H) 9.26 (s, 1 H) 11.95 (s, 1 H); HRMS (ESI+) calcd for C₂₂H₂₄N₈ (MH+) 401.21967, found 401.2205.

### Example 262

### 4-[(3,4-dimethylisoxazol-5-yl)amino]-6-[(1H-imidazol-5-ylmethyl)amino]quinoline-3-carbonitrile

Step 1: Following the procedure described above in Example 229, 4-chloro-6-nitro-quinoline-3-carbonitrile (0.30 g, 1.28 mmol) was reacted with 3,4-dimethyl-isoxazol-5-ylamine (0.17 g, 1.54 mmol) in 4 mL EtOH. After work up, 4-(3,4-dimethyl-isoxazol-5-ylamino)-6-nitro-quinoline-3-carbonitrile was obtained as a red solid (0.26 g, 52% purify by LC/MS, 34% yield), and was used in the next step without further purification.

Step 2: Following the procedure described above in Example 261, the product from the previous step (0.26 g, 0.80 mmol) was reacted with tin chloride dihydrate (0.95 g, 4.2 mmol) in 6 mL EtOH. After work up, 6-amino-4-(3,4-dimethyl-isoxazol-5-ylamino)-quinoline-3-carbonitrile was obtained as a yellow solid(0.15 g, 64% yield).

Step 3: Following the procedure described above in Example 4, 6-amino-4-(3,4-dimethyl-isoxazol-5-ylamino)-quinoline-3-carbonitrile (150 mg, 0.54 mmol) was reacted with 4(5)-imidazolecarboxyaldehyde (67.1 mg, 0.70 mmol) and NaCNBH₃ (23.8 mg, 0.38 mmol) in 7 mL EtOH. The crude product was purified by preparative HPLC, and lyophilized to give the product as a yellow solid (13.9 mg, 7%): 1H NMR (400 MHz, DMSO-D6) δ ppm 1.78 (s, 3 H) 2.11-2.32 (m, 3 H) 4.29 (d, J=2.53 Hz, 2 H) 6.65 (s, 1 H) 7.09 (s, 1 H) 7.27 (s, 1 H) 7.43 (s, 1 H) 7.68 (s, 2 H) 8.40 (s, 1 H) 9.91 (br, s,1 H) 12.02 (br, s,1 H); HRMS (ESI+) calcd for C₁₉H₁₇N₇O (MH+) 360.15673, found 360.1573.

### Example 263

### 6-[(1H-imidazol-5-ylmethyl)amino]-4-(pyridin-3-ylamino)quinoline-3-carbonitrile

Following the procedure described above in Example 262, 6-amino-4-(pyridin-3-ylamino)-quinoline-3-carbonitrile (140 mg, 0.54 mmol) with 4(5)-imidazolecarboxyaldehyde (67.3 mg, 0.70 mmol) and NaCNBH₃ (23.8 mg, 0.38 mmol) in 5 mL EtOH. The crude product was purified by preparative HPLC, and lyophilized to give the product as a yellow solid (26.0 mg, 14%): 1H NMR (400 MHz, DMSO-D6) δ ppm 4.42 (d, J=5.05 Hz, 2 H) 6.73 (t, J=4.80 Hz, 1 H) 7.14-7.26 (m, 1 H) 7.38 (d, J=2.27 Hz, 1 H) 7.53-7.61 (m, 2 H) 7.79 (d, J=1.01 Hz, 2 H) 7.87 (d, J=9.09 Hz, 1 H) 8.46-8.58 (m, 2 H) 8.69 (d, J=2.53 Hz, 1 H) 9.56 (s, 1 H) 12.14 (br, s,1 H); HRMS (ESI+) calcd for C₁₉H₁₅N₇ (MH+) 342.14617, found 342.1467.

### Example 264

### 6-[(1H-imidazol-5-ylmethyl)amino]-4-(pyridin-4-ylamino)quinoline-3-carbonitrile

Following the procedure described above in Example 262, 6-amino-4-(pyridin-4-ylamino)-quinoline-3-carbonitrile (114 mg, 0.44 mmol) with 4(5)-imidazolecarboxyaldehyde (54.5 mg, 0.57 mmol) and NaCNBH₃ (19.2 mg, 0.31 mmol) in 5 mL EtOH. The crude product was purified by preparative HPLC, and lyophilized to give the product as a dark yellow solid (13.8 mg, 9%): 1H NMR (400 MHz, DMSO-D6) δ ppm 4.20 (d, J=5.05 Hz, 2 H) 6.75 (s, 1 H) 6.88 (s, 2 H) 6.94-7.01 (m, 2 H) 7.47 (dd, J=9.09, 2.27 Hz, 1 H) 7.60 (s, 1 H) 7.81 (d, J=9.09 Hz, 1 H) 8.31 (s, 2 H) 8.59 (s, 1 H) 9.61 (br, s, 1 H) 11.94 (br, s, 1 H); HRMS (ESI+) calcd for C₁₉H₁₅N₇ (MH+) 342.14617, found 342.1467.

### Example 265

### 4-[(3-Chloro-4-fluorophenyl)amino]-8-fluoro-6-[(1H-imidazol-5-ylmethyl)amino]quinoline-3-carbonitrile

Step 1: A 250 mL round-bottomed flask was charged with 2-fluoro-4-nitro-phenylamine (5.0 g, 32.0 mmol), ethyl (ethoxymethylene)cyanoacetate (5.96 g, 35.2 mmol) and 60 mL DMF. The mixture was stirred vigorously to dissolve both reagents, Cs₂CO₃ (20.86 g, 64.0 mmol) was added, and the reaction mixture was stirred at RT for 3.5 hours. To work up, the contents of the flask were poured into 500 mL water and the precipitate collected by suction filtration, washed three times with water, then washed twice with ether, and dried under vacuum to give an orange solid as crude, and was purified by dissolving the crude in 60 mL DMF, then 800 mL EtOAc was added. The solution was then washed with brine twice (2×200 mL), separated. The organic layer was dried over Na₂SO₄, filtered, concentrated down to a brown-red solid. Ether was added, the resulting suspension was filtered, washed with ether, dried under high vacuum overnight to give 2-cyano-3-(2-fluoro-4-nitro-phenylamino)-acrylic acid ethyl ester as a brown-red solid (6.6 g, 74% yield): (50%) 1H NMR (400 MHz, DMSO-D6) δ ppm 1.21 (t, J=7.07 Hz, 3 H) 4.11 (q, J=7.07 Hz, 2 H) 7.35 (q, 1 H) 7.95-8.12 (m, 3 H); (50%) 1H NMR (400 MHz, DMSO-D6) □ ppm 1.21 (t, J=7.07 Hz, 3 H) 4.28 (q, J=7.07 Hz, 2 H) 8.19 (q, J=8.84 Hz, 1 H) 8.32 (dd, 10.99, 2.40 Hz, 1 H) 8.39 (s, 1 H) 8.82 (s, 1 H) 11.00 (s, 1 H).

Step 2: Following the procedure described above in Example 229, the product from the previous step (6.48 g, 23.0 mmol) was taken up in 300 mL Dowtherm and heated at reflux for 3 hours under argon. After work up, 8-fluoro-6-nitro-4-oxo-1,4-dihydroquinoline-3-carbonitrile was obtained as a brown solid (2.17 g, 40%): 1H NMR (400 MHz, DMSO-D6) δ ppm 8.49 (dd, J=10.48, 2.40 Hz, 1 H) 8.55-8.62 (m, 1 H) 8.76 (s, 1 H) 13.40 (br, s, 1 H); HRMS (ESI+) calcd for C₁₀H₄FN₃O₃ (MH+) 234.03095, found 234.0308.

Step 3: In a 100 mL round-bottomed flask equipped with a condenser, the product from step 2 (2.1 g, 8.9 mmol) was taken up in 30 mL POCl₃ and heated at reflux for 7.5 hours. The reaction mixture was then stirred at RT overnight, and then POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum overnight to give 4-chloro-8-fluoro-6-nitro-quinoline-3-carbonitrile as a brown solid (2.23 g, 100% yield):

1H NMR (400 MHz, DMSO-D6) δ ppm 8.71 (dd, J=9.85, 2.27 Hz, 1 H) 8.86-8.92 (m, 1 H) 9.46 (s, 1 H).

Step 4: Following the procedure described above in Example 229, 4-chloro-8-fluoro-6-nitro-quinoline-3-carbonitrile (0.60 g, 2.38 mmol) was reacted with 3-chloro-4-fluoroaniline (0.42 g, 2.86 mmol) in 10 mL EtOH. After work up, 4-[(3-chloro-4-fluorophenyl)amino]-8-fluoro-6-nitroquinoline-3-carbonitrile was obtained as a brown solid (0.7 g, 82% yield): 1H NMR (400 MHz, DMSO-D6) δ ppm 7.51-7.58 (m, 1 H) 7.62 (t, J=8.97 Hz, 1 H) 7.82 (dd, J=6.44, 2.15 Hz, 1 H) 8.59 (dd, J=10.11, 2.02 Hz, 1 H) 8.91 (s, 1 H) 9.52 (s, 1 H) 10.73 (s, 1 H); HRMS (ESI+) calcd for C₁₆H₇ClF₂N₄O₂ (MH+) 361.02983, found 361.0294.

Step 5: Following the procedure described above in Example 229, 4-[(3-chloro-4-fluorophenyl)amino]-8-fluoro-6-nitroquinoline-3-carbonitrile (1.55 g, 4.30 mmol) was reacted with tin chloride dihydrate (4.85 g, 21.5 mmol) in 60 mL EtOH. After work up, 6-amino-4-[(3-chloro-4-fluorophenyl)amino]-8-fluoroquinoline-3-carbonitrile was obtained as a light brown solid (1.42 g, 100% yield): 1H NMR (400 MHz, DMSO-D6) δ ppm 5.94 (s, 2 H) 6.98-7.10 (m, 2 H) 7.15-7.29 (m, 1 H) 7.35-7.48 (m, 2 H) 8.26-8.39 (m, 1 H) 9.45 (br, s,1 H); HRMS (ESI+) calcd for C₁₆H₉ClF₂N₄ (MH+) 331.05566, found 331.0562.

Step 6: Following the procedure described above in Example 4, 6-amino-4-[(3-chloro-4-fluorophenyl)amino]-8-fluoroquinoline-3-carbonitrile (70 mg, 0.21 mmol) was reacted with 4(5)-imidazolecarboxyaldehyde (28.5 mg, 0.30 mmol) and NaCNBH₃ (9.3 mg, 0.15 mmol) in 4 mL EtOH. The crude product was purified by preparative HPLC, and lyophilized to give the product as a bright yellow solid (26.7 mg, 31%): 1H NMR (400 MHz, DMSO-D6) δ ppm 4.19 (d, J=5.05 Hz, 2 H) 6.61 (t, J=5.05 Hz, 1 H) 6.95-7.07 (m, 2 H) 7.15 (d, J=12.88, 2.02 Hz, 1 H) 7.19-7.28 (m, 1 H) 7.38 (t, J=9.09 Hz, 1 H) 7.48 (dd, J=6.57, 2.53 Hz, 1 H) 7.56 (d, J=1.01 Hz, 1 H) 8.11 (s, 1 H) 8.23 (s, 1 H) 9.40 (s, 1 H); HRMS (ESI+) calcd for C₂₀H₁₃ClF₂N₆ (MH+) 411.09310, found 411.0925.

### Example 266

### 4-[(3-chloro-4-fluorophenyl)amino]-8-fluoro-6-{[(1-oxidopyridin-2-yl)methyl]amino}quinoline-3-carbonitrile

Following the procedure described above in Example 4, 6-amino-4-[(3-chloro-4-fluorophenyl)amino]-8-fluoroquinoline-3-carbonitrile (70 mg, 0.21 mmol) was reacted with 1-oxy-pyridine-2-carbaldehyde (54.6 mg, 0.44 mmol) and NaCNBH₃ (9.3 mg, 0.15 mmol) in 4 mL EtOH. The crude product was purified by preparative HPLC, and lyophilized to give the product as a yellow solid (5.6 mg, 6%): 1H NMR (400 MHz, DMSO-D6) δ ppm 4.52 (d, J=6.06 Hz, 2 H) 6.86-7.08 (m, 2 H) 7.12-7.41 (m, 6 H) 7.41-7.53 (m, 1 H) 8.17-8.35 (m, 2 H) 9.38 (s, 1 H); HRMS (ESI+) calcd for C₂₂H₁₄ClF₂N₅O (MH+) 438.09277, found 438.092.

### Example 267

### 4-[(3-chloro-4-fluorophenyl)amino]-8-fluoro-6-[(1H-pyrazol-5-ylmethyl)amino]quinoline-3-carbonitrile

Following the procedure described above in Example 4, 6-amino-4-[(3-chloro-4-fluorophenyl)amino]-8-fluoroquinoline-3-carbonitrile (80 mg, 0.24 mmol) was reacted with 2H-pyrazole-3-carbaldehyde (34.6 mg, 0.36 mmol) and NaCNBH₃ (10.6 mg, 0.17 mmol) in 4 mL EtOH. The crude product was purified by preparative HPLC, and lyophilized to give the product as a bright yellow solid (10.3 mg, 10%):

1H NMR (400 MHz, DMSO-D6) δ ppm 4.29 (d, J-5.05 Hz, 2 H) 6.13-6.24 (m, J=2.02 Hz, 1 H) 6.71 (d, J=6.57 Hz, 1 H) 7.05 (s, 1 H) 7.11-7.19 (m, 1 H) 7.19-7.27 (m, 1 H) 7.30-7.42 (m, 1 H) 7.47 (dd, J=6.69, 2.40 Hz, 2 H) 7.56 (s, 1 H) 8.24 (s, 1 H) 9.43 (s, 1 H); HRMS (ESI+) calcd for C₂₀H₁₃ClF₂N₆ (MH+) 411.09310, found 411.0932.

### Example 268

### 4-[(3-Chloro-4-fluorophenyl)amino]-8-fluoro-6-{[(1-methyl-1H-imidazol-2-yl)methyl]amino}quinoline-3-carbonitrile

Following the procedure described above in Example 4, 6-amino-4-[(3-chloro-4-fluorophenyl)amino]-8-fluoroquinoline-3-carbonitrile (80 mg, 0.24 mmol) was reacted with 1-methyl-1H-imidazole-2-carbaldehyde (39.6 mg, 0.36 mmol) and NaCNBH₃ (10.6 mg, 0.17 mmol) in 5 mL EtOH. The crude product was purified by preparative HPLC, and lyophilized to give the product as a bright yellow solid (39.3 mg, 39%): 1H NMR (400 MHz, DMSO-D6) δ ppm 3.58 (s, 3 H) 4.33 (d, J=4.80 Hz, 2 H) 6.69-6.83 (m, 2 H) 7.01-7.14 (m, 2 H) 7.19-7.26 (m, 2 H) 7.38 (t, J=9.09 Hz, 1 H) 7.47 (dd, J=6.57, 2.53 Hz, 1 H) 8.26 (s, 1 H) 9.41 (s, 1 H); HRMS (ESI+) calcd for C₂₁H₁₅ClF₂N₆ (MH+) 425.10875, found 425.1094.

### Example 269

### 4-[(3-chloro-4-fluorophenyl)amino]-6-{[(1,5-dimethyl-1H-imidazol-4-yl)methyl]amino}-8-fluoroquinoline-3-carbonitrile

Following the procedure described above in Example 4, 6-amino-4-[(3-chloro-4-fluorophenyl)amino]-8-fluoroquinoline-3-carbonitrile (80 mg, 0.24 mmol) was reacted with 1-methyl-1H-imidazole-2-carbaldehyde (35.8 mg, 0.29 mmol) and NaCNBH₃ (10.6 mg, 0.17 mmol) in 5 mL EtOH. The crude product was purified by preparative HPLC, and lyophilized to give the product as a bright yellow solid (13.2 mg, 13%): 1H NMR (400 MHz, DMSO-D6) δ ppm 2.27 (s, 3 H) 3.62 (s, 3 H) 4.23 (d, J=4.55 Hz, 2 H) 6.59 (t, J=4.04 Hz, 1 H) 7.16 (d, J=1.52 Hz, 1 H) 7.34 (d, J=13.14, 2.02 Hz, 1 H) 7.37-7.45 (m, 1 H) 7.56 (t, J=8.97 Hz, 1 H) 7.60-7.68 (m, 2 H) 8.41 (s, 1 H) 9.54 (s, 1 H); HRMS (ESI+) calcd for C₂₂H₁₇ClF₂N₆ (MH+) 439.12440, found 439.1248.

### Example 270

### 4-[(3-Chloro-4-fluorophenyl)amino]-8-fluoro-6-{[(1-oxidopyridin-4-yl)methyl]amino}quinoline-3-carbonitrile

Step 1: In a microwave vial, (1-oxy-pyridin-4-yl)-methanol (0.45 g, 3.60 mmol) was taken up in 2 mL each CH₂Cl₂ and 1,4-dioxane, and activated MnO₂ (1.09 g, 12.6 mmol) was added. The vial was crimp-sealed and heated in a microwave reactor at 140° C. for 5 minutes, until LC-MS analysis showed complete disappearance of the starting material. The contents of the vial were then rinsed into a 500 mL Erlenmeyer flask and stirred with 200 mL H₂O for 30 minutes. The suspension was then filtered to remove MnO₂, and evaporated to give product isonicotinaldehyde 1-oxide of sufficient purity to be used in the next step (0.44 g, 99% yield): 1H NMR (400 MHz, DMSO-D6) δ ppm 7.90 (d, J=5.56 Hz, 2 H) 8.41 (d, J=5.81 Hz, 2 H) 9.97 (s, 1 H).

Step 2: Following the procedure described above in Example 4, 6-amino-4-[(3-chloro-4-fluorophenyl)amino]-8-fluoroquinoline-3-carbonitrile (80 mg, 0.24 mmol) was reacted with 1-oxy-pyridine-4-carbaldehyde (44.3 mg, 0.36 mmol) and NaCNBH₃ (10.6 mg, 0.17 mmol) in 5 mL EtOH. The crude product was purified by preparative HPLC, and lyophilized to give the product as a yellow solid (9.2 mg, 9%): 1H NMR (400 MHz, DMSO-D6) δ ppm 4.49 (d, J=6.06 Hz, 2 H) 7.04-7.11 (m, 1 H) 7.16 (t, J=6.19 Hz, 1 H) 7.24 (dd, J=12.63, 2.02 Hz, 1 H) 7.31-7.39 (m, 1 H) 7.43 (d, J=6.82 Hz, 2 H) 7.51 (t, J=8.97 Hz, 1 H) 7.59 (dd, J=6.44, 2.65 Hz, 1 H) 8.24 (d, J=6.82 Hz, 2 H) 8.39 (s, 1 H) 9.47 (s, 1 H); HRMS (ESI+) calcd for C₂₂H₁₄ClF₂N₅O (MH+) 438.09277, found 438.0929.

### Example 271

### Preparation of 8-Chloro-4-[(3-chloro-4-fluorophenyl)amino]-6-{[(1-oxidopyridin-4-yl)methyl]amino}quinoline-3-carbonitrile

Step 1: Following the procedure described above in Example 229, 4-chloro-8-chloro-6-nitro-quinoline-3-carbonitrile (2.0 g, 7.46 mmol) was reacted with 3-chloro-4-fluoroaniline (1.3 g, 8.95 mmol) in 30 mL EtOH. After work up, 8-chloro-4-(3-chloro-4-fluoro-phenylamino)-6-nitro-quinoline-3-carbonitrile was obtained as a yellow solid (2.0 g, 71% yield).

Step 2: Following the procedure described above in Example 229, 8-chloro-4-(3-chloro-4-fluoro-phenylamino)-6-nitro-quinoline-3-carbonitrile (1.73 g, 4.59 mmol) was reacted with tin chloride dihydrate (4.14 g, 18.35 mmol) in 70 mL EtOH. After work up, 6-amino-8-chloro-4-(3-chloro-4-fluoro-phenylamino)-quinoline-3-carbonitrile was obtained as a brown solid (1.4 g, 88% yield).

Step 3: Following the procedure described above in Example 4, 6-amino-4-[(3-chloro-4-fluorophenyl)amino]-8-chloroquinoline-3-carbonitrile (80 mg, 0.23 mmol) was reacted with 1-oxy-pyridine-4-carbaldehyde (42.5 mg, 0.35 mmol) and NaCNBH₃ (10.1 mg, 0.16 mmol) in 4 mL EtOH. The crude product was purified by preparative HPLC, and lyophilized to give the product as a yellow solid (10.7 mg, 10%): 1H NMR (400 MHz, DMSO-D6) δ ppm 4.36 (d, J=5.81 Hz, 2 H) 7.03 (t, J=6.06 Hz, 1 H) 7.08 (d, J=2.27 Hz, 1 H) 7.17-7.24 (m, 1 H) 7.30 (d, J=7.07 Hz, 2 H) 7.37 (t, J=8.97 Hz, 1 H) 7.45 (dd, J=6.57, 2.53 Hz, 1 H) 7.48 (d, J=2.27 Hz, 1 H) 8.10 (d, J=7.07 Hz, 2 H) 8.34 (s, 1 H) 9.35(s, 1 H); HRMS (ESI+) calcd for C₂₂H₁₄Cl₂FN₅O (MH+) 454.06322, found 454.0631.

### Example 272

### 8-Chloro-4-(3-chloro-4-fluoro-phenylamino)-6-[(pyrimidin-5-ylmethyl)-amino]-quinoline-3-carbonitrile

Following the procedure described above in Example 4, 6-amino-4-[(3-chloro-4-fluorophenyl)amino]-8-chloroquinoline-3-carbonitrile (80 mg, 0.23 mmol) was reacted with pyrimidine-5-carbaldehyde (52.3 mg, 0.48 mmol) and NaCNBH₃ (10.1 mg, 0.16 mmol) in 5 mL EtOH. The crude product was purified by preparative HPLC, and lyophilized to give the product as a yellow solid (32.8 mg, 33%): 1H NMR (400 MHz, DMSO-D6) δ ppm 4.47 (d, J=5.81 Hz, 2 H) 7.04 (t, J=5.81 Hz, 1 H) 7.22 (d, J=2.53 Hz, 1 H) 7.24-7.32 (m, 1 H) 7.43 (t, J=8.97 Hz, 1 H) 7.49-7.58 (m, 2 H) 8.41 (s, 1 H) 8.84 (s, 2 H) 9.11 (s, 1 H) 9.45 (s, 1 H); HRMS (ESI+) calcd for C₂₁H₁₃Cl₂FN₆ (MH+) 439.06355, found 439.0627.

### Example 273

### 8-Chloro-4-[(3-chloro-4-fluorophenyl)amino]-6-{[4-methoxy-3-(2-morpholin-4-ylethoxy)benzyl]amino}quinoline-3-carbonitrile

Following the procedure described above in Example 4, 6-amino-4-[(3-chloro-4-fluorophenyl)amino]-8-chloroquinoline-3-carbonitrile (70 mg, 0.20 mmol) was reacted with 4-methoxy-3-(2-morpholin-4-yl-ethoxy)-benzaldehyde (79.6 mg, 0.30 mmol) and NaCNBH₃ (8.8 mg, 0.14 mmol) in 4 mL EtOH. The crude product was purified by preparative HPLC, and lyophilized to give the product as a yellow solid (53.5 mg, 45%): 1H NMR (400 MHz, DMSO-D6) δ ppm 2.39-2.45 (m, 4 H) 2.62 (t, J=5.81 Hz, 2 H) 3.54 (t, 4 H) 3.72 (s, 3 H) 4.01 (t, J=5.94 Hz, 2 H) 4.29 (d, J=5.56 Hz, 2 H) 6.91 (d, 3 H) 7.07 (s, 1 H) 7.19 (d, J=2.27 Hz, 1 H) 7.22-7.30 (m, 1 H) 7.44 (t, J=8.97 Hz, 1 H) 7.50 (dd, J=6.57, 2.78 Hz, 1 H) 7.54 (d, J=2.27 Hz, 1 H) 8.38 (s, 1 H) 9.44 (s, 1 H); HRMS (ESI+) calcd for C₃₀H₂₈Cl₂FN₅O₃ (MH+) 596.16260, found 596.1622.

### Example 274

### 8-chloro-4-[(3-chloro-4-fluorophenyl)amino]-6-{[(6-morpholin-4-ylpyridin-2-yl)methyl]amino}quinoline-3-carbonitrile

Following the procedure described above in Example 4, 6-amino-4-[(3-chloro-4-fluorophenyl)amino]-8-chloroquinoline-3-carbonitrile (80 mg, 0.23 mmol) was reacted with 6-morpholin-4-yl-pyridine-2-carbaldehyde (49.0 mg, 0.25 mmol) and NaCNBH₃ (10.1 mg, 0.16 mmol) in 4 mL EtOH. The crude product was purified by preparative HPLC, and lyophilized to give the product as a yellow solid (55.3 mg, 46%): 1H NMR (400 MHz, DMSO-D6) δ ppm 3.37-3.51 (m, 4 H) 3.61-3.73 (m, 4 H) 4.36 (d, J=6.06 Hz, 2 H) 6.68 (t, J=7.83 Hz, 2 H) 6.92 (t, J=6.06 Hz, 1 H) 7.17-7.31 (m, 2 H) 7.42 (t, J=8.97, 1 H) 7.45-7.55 (m, 2 H) 7.65 (d, J=2.27 Hz, 1 H) 8.39 (s, 1 H) 9.46 (s, 1 H); HRMS (ESI+) calcd for C₂₆H₂₁Cl₂FN₆O (MH+) 523.12107, found 523.1207.

### Example 275

### 8-Chloro-4-[(3-chloro-4-fluorophenyl)amino]-6-({[1-oxido-6-(trifluoromethyl)pyridin-3-yl]methyl}amino)quinoline-3-carbonitrile

Step 1: In a 50 mL round-bottomed flask, 6-trifluoromethyl-nicotinic acid (0.50 g, 2.62 mmol) was taken up in 6 mL dry THF and cooled down to 0° C. Then solid LiAlH₄ was added in 3 portions (3×33.1 mg, 2.62 mmol) at 0° C. The reaction mixture was then allowed to warm up to RT and stirred at RT for 2 days, until TLC analysis showed complete disappearance of starting material. And the reaction mixture was quenched with 1N NaOH solution at 0° C. Then extracted the mixture with EtOAc 3 times, the combined EtOAc layers were dried over Na₂SO₄, filtered and concentrated down to give the product (6-trifluoromethyl-pyridin-3-yl)-methanol as a yellow oil (0.33 g, 70% yield): 1H NMR (400 MHz, DMSO-D6) δ ppm 4.65 (s, 2 H) 5.53 (s, 1 H) 7.87 (d, J=8.08 Hz, 1 H) 8.00 (d, J=8.08 Hz, 1 H) 8.70 (s, 1 H).

Step 2: In a 50 mL round-bottomed flask equipped with a condenser, the product from the previous step (0.33 g, 1.8 mmol) was taken up in 10 mL CH₂Cl₂/MeOH (9:1, v/v). Then MMPP (magnesium monoperoxyphthalate hexahydrate) (2.96 g, 6.0 mmol) was added. The reaction mixture was heated at reflux for 2 days under nitrogen and then allowed it to cool to RT. The white suspension was filtered, washed with CH₂Cl₂, the filtrate obtained was concentrated down to give a liquid as crude. The crude product was purified by preparative HPLC to give the product (1-oxy-6-trifluoromethyl-pyridin-3-yl)-methanol as a colorless liquid (30.0 mg, 9% yield): 1H NMR (400 MHz, DMSO-D6) δ ppm 4.56 (d, J=5.56 Hz, 2 H) 5.63 (t, J=5.81 Hz, 1 H) 7.44 (d, J=8.34 Hz, 1 H) 7.91 (d, J=8.34 Hz, 1 H) 8.35 (s, 1 H).

Step 3: In a microwave vial, the product from step 2 (30.0 mg, 0.16 mmol) was taken up in 1 mL each CH₂Cl₂ and 1,4-dioxane, and activated MnO₂ (47.3 mg, 0.54 mmol) was added. The vial was crimp-sealed and heated in a microwave reactor at 120° C. for 35 minutes, until LC-MS analysis showed complete disappearance of starting material. The contents of the vial were then rinsed into a 500 mL Erlenmeyer flask and stirred with 50 mL H₂O for 30 minutes. The suspension was then filtered to remove MnO₂, and evaporated to give product 1-oxy-6-trifluoromethyl-pyridine-3-carbaldehyde of sufficient purity to be used in the next step (30.0 mg, 100% yield).

Step 4: Following the procedure described above in Example 4, 6-amino-4-[(3-chloro-4-fluorophenyl)amino]-8-chloroquinoline-3-carbonitrile (70 mg, 0.20 mmol) was reacted with 1-oxy-6-trifluoromethyl-pyridine-3-carbaldehyde (38.2 mg, 0.20 mmol) and NaCNBH₃ (8.8 mg, 0.14 mmol) in 4 mL EtOH. The crude product was purified by preparative HPLC under basic condition, and lyophilized to give a yellow, solid as Et₃N salt form (4.0 mg,1%): 1H NMR (400 MHz, DMSO-D6) δ ppm 4.50 (d, J=6.06 Hz, 2 H) 7.02-7.11 (m, 1 H) 7.16-7.28 (m, 2 H) 7.40 (t, J=9.09 Hz, 1 H) 7.46 (s, 2 H) 7.52 (s, 1 H) 7.92 (d, J=8.34 Hz, 1 H) 8.36 (s, 1 H) 8.46 (s, 1 H) 9.39 (s, 1 H); HRMS (ESI+) calcd for C₂₃H₁₃Cl₂F₄N₅O (MH+) 522.05060, found 522.0502.

### Example 276

### 8-chloro-4-[(3-chloro-4-fluorophenyl)amino]-6-{[(4-methoxy-3,5-dimethylpyridin-2-yl)methyl]amino}quinoline-3-carbonitrile

Step 1: In a microwave vial, (4-methoxy-3,5-dimethyl-pyridin-2-yl)-methanol (0.20 g, 1.2 mmol) was taken up in 1 mL each CH₂Cl₂ and 1,4-dioxane, and activated MnO₂ (0.36 g, 4.2 mmol) was added. The vial was crimp-sealed and heated in a microwave reactor at 140° C. for 7 minutes, until LC-MS analysis showed complete disappearance of starting material. The contents of the vial were then rinsed into a 500 mL Erlenmeyer flask and stirred with 100 mL H₂O for 30 minutes. The suspension was then filtered to remove MnO₂, and evaporated to give product 4-methoxy-3,5-dimethyl-pyridine-2-carbaldehyde of sufficient purity to be used in the next step (40.0 mg, 33% yield).

Step 2: Following the procedure described above in Example 4, 6-amino-4-[(3-chloro-4-fluorophenyl)amino]-8-chloroquinoline-3-carbonitrile (70 mg, 0.20 mmol) was reacted with 4-methoxy-3,5-dimethyl-pyridine-2-carbaldehyde (105.0 mg, 0.64 mmol) and NaCNBH₃ (8.8 mg, 0.14 mmol) in 4 mL EtOH. The crude product was purified by preparative HPLC, and lyophilized to give the product as a yellow solid (42.0 mg, 42%): 1H NMR (400 MHz, DMSO-D6) δ ppm 2.23 (d, J=5.81 Hz, 6 H) 3.74 (s, 3 H) 4.38 (d, J=4.04 Hz, 2 H) 6.85 (t, J=3.79 Hz, 1 H) 7.22 (d, J=2.53 Hz, 1 H) 7.27-7.33 (m, 1 H) 7.45 (t, J=9.09 Hz, 1 H) 7.50-7.57 (m, 1 H) 7.75 (d, J=2.53 Hz, 1 C₂₅H₂₀Cl₂FN₅O (MH+) 496.11017, found 496.1095.

### Example 277

### 8-Chloro-4-[(3-chloro-4-fluorophenyl)amino]-6-{[(4-methoxy-3,5-dimethyl-1-oxidopyridin-2-yl)methyl]amino}quinoline-3-carbonitrile

Step 1: In a 100 mL round-bottomed flask, (4-methoxy-3,5-dimethyl-pyridin-2-yl)-methanol (1.0 g, 6.0 mmol) was taken up in 50 mL CH₂Cl₂/MeOH (9:1, v/v). Then MMPP (magnesium monoperoxyphthalate hexahydrate) (5.92 g, 12.0 mmol) was added. The reaction mixture was stirred at RT overnight under nitrogen, then filtered and washed with CH₂Cl₂. The filtrate obtained was concentrated down to give a light yellow gum as crude. The crude product was purified by preparative HPLC to give the product (4-methoxy-3,5-dimethyl-1-oxy-pyridin-2-yl)-methanol as a colorless sticky oil (0.65 g, 59% yield): 1H NMR (400 MHz, DMSO-D6) δ ppm 2.16 (s, 3 H) 2.24 (s, 3 H) 3.70 (s, 3 H) 4.69 (s, 2 H) 8.11 (s, 1 H).

Step 2: In a microwave vial, the product from the previous step (0.16 g, 0.86 mmol) was taken up in 1 mL each CH₂Cl₂ and 1,4-dioxane, and activated MnO₂ (0.26 g, 3.0 mmol) was added. The vial was crimp-sealed and heated in a microwave reactor at 140° C. for 8 minutes, until LC-MS analysis showed complete disappearance of starting material. The contents of the vial were then rinsed into a 500 mL Erlenmeyer flask and stirred with 100 mL H₂O for 30 minutes. The suspension was then filtered to remove MnO₂, and evaporated to give product 4-methoxy-3,5-dimethyl-1-oxy-pyridine-2-carbaldehyde of sufficient purity to be used in the next step (0.15 g, 99% yield): 1H NMR (400 MHz, DMSO-D6) δ ppm 2.37-2.89 (m, 6 H) 3.73 (s, 3 H) 8.24 (s, 1 H) 10.29 (s, 1 H).

Step 3: Following the procedure described above in Example 4, 6-amino-4-[(3-chloro-4-fluorophenyl)amino]-8-chloroquinoline-3-carbonitrile (80 mg, 0.23 mmol) was reacted with 4-methoxy-3,5-dimethyl-1-oxy-pyridine-2-carbaldehyde (83.5 mg, 0.46 mmol) and NaCNBH₃ (10.1 mg, 0.16 mmol) in 5 mL EtOH. The crude product was purified by preparative HPLC, and lyophilized to give the product as a yellow solid (23.0 mg, 20%): 1H NMR (400 MHz, DMSO-D6) δ ppm 2.18 (s, 3 H) 2.28 (s, 3 H) 3.70 (s, 3 H) 4.61 (d, J=5.05 Hz, 2 H) 6.52 (s, 1 H) 6.70-6.81 (m, 1 H) 7.31 (s, 1 H) 7.45 (t, J=9.09 Hz, 1 H) 7.47-7.61 (m, 2 H) 8.19 (s, 1 H) 8.39 (s, 1 H) 9.43 (s, 1 H); HRMS (ESI+) calcd for C₂₅H₂₀Cl₂FN₅O₂ (MH+) 512.10508, found 512.1044.

### Example 278

### 8-Chloro-4-[(3-chloro-4-fluorophenyl)amino]-6-[(quinolin-4-ylmethyl)amino]quinoline-3-carbonitrile

Following the procedure described above in Example 4, 6-amino-4-[(3-chloro-4-fluorophenyl)amino]-8-chloroquinoline-3-carbonitrile (80 mg, 0.23 mmol) was reacted with quinoline-4-carbaldehyde (108.4 mg, 0.70 mmol) and NaCNBH₃ (10.1 mg,0.16 mmol) in 4 mL EtOH. The crude product was purified by preparative HPLC, and lyophilized to give the product as a yellow solid (13.8 mg, 12%): 1H NMR (400 MHz, DMSO-D6) δ ppm 4.94 (d, J=5.81 Hz, 2 H) 7.14 (t, J=6.19 Hz, 1 H) 7.19-7.26 (m, 2 H) 7.36 (t, J=9.09 Hz, 1 H) 7.42-7.51 (m, 2 H) 7.63 (d, J=2.27 Hz, 1 H) 7.67 (t, J=7.71 Hz, 1 H) 7.80 (t, J=6.95 Hz, 1 H) 8.05-8.09 (m, 1 H) 8.18 (d, J=8.34 Hz, 1 H) 8.41 (s, 1 H) 8.84 (d, J=4.29 Hz, 1 H) 9.39 (s, 1 H); HRMS (ESI+) calcd for C₂₆H₁₆Cl₂FN₅ (MH+) 488.08395, found 488.0832.

### Example 279

### 8-Chloro-4-[(3-chloro-4-fluorophenyl)amino]-6-[(1 H-tetrazol-5-ylmethyl)amino]quinoline-3-carbonitrile

Step 1: In a 100 mL round-bottomed flask, diethoxy acetonnitrile (1.0 g, 7.7 mmol) was taken up in 35 mL DEE. Then azidotributyltin (3.34 g, 10.1 mmol) was added. The reaction mixture was heated at reflux overnight under nitrogen, then evaporated the solvent in vacuo to a black residue. 20 mL 1.25M HCl in MeOH was added to the residue, the mixture was heated at reflux for 3 hours, then allowed it cool down to RT and evaporated down solvent to give a black oil as crude, and was used in the next step directly without further purification.

Step 2: Following the procedure described above in Example 4, 6-amino-4-[(3-chloro-4-fluorophenyl)amino]-8-chloroquinoline-3-carbonitrile (200 mg, 0.58 mmol) was reacted with 1H-tetrazole-5-carbaldehyde (141.2 mg, 1.44 mmol) and NaCNBH₃ (25.3 mg, 0.40 mmol) in 5 mL EtOH. The crude product was purified by preparative HPLC, and lyophilized to give the product as a yellow solid (17.0 mg, 7%): 1H NMR (400 MHz, DMSO-D6) δ ppm 4.45 (d, J=5.05 Hz, 2 H) 6.74 (t, J=4.93 Hz, 1 H) 7.26 (d, J=2.27 Hz, 1 H) 7.28-7.34 (m, 1 H) 7.45 (t, J=9.09 Hz, 1 H) 7.54 (dd, J=6.57, 2.78 Hz, 1 H) 7.68 (d, J=2.27 Hz, 1 H) 8.37 (s, 1 H) 9.54 (s, 1 H); HRMS (ESI+) calcd for C₁₈H₁₁Cl₂FN₈ (MH+) 429.05405, found 429.0539.

### Example 280

### 8-chloro-4-[(3-chloro-4-fluorophenyl)amino]-6-({[4-(methylamino)-2-(methylthio)pyrimidin-5-yl]methyl]amino)quinoline-3-carbonitrile

Following the procedure described above in Example 4, 6-amino-4-[(3-chloro-4-fluorophenyl)amino]-8-chloroquinoline-3-carbonitrile (80 mg, 0.23 mmol) was reacted with 4-methylamino-2-methylsulfanyl-pyrimidine-5-carbaldehyde (59.0 mg,0.32 mmol) and NaCNBH₃ (10.1 mg, 0.16 mmol) in 4 mL EtOH. The crude product was purified by preparative HPLC, and lyophilized to give the product as a yellow solid (34.1 mg, 29%): 1H NMR (400 MHz, DMSO-D6) δ ppm 2.42 (s, 3 H) 2.86 (d, J=4.55 Hz, 3 H) 4.11 (d, J=4.55 Hz, 2 H) 6.65 (t, J=5.05 Hz, 1 H) 7.09 (t, J=4.80 Hz, 1 H) 7.18 (d, J=2.27 Hz, 1 H) 7.24-7.32 (m, 1 H) 7.44 (t, J=8.97 Hz, 1 H) 7.49-7.56 (m, 2 H) 7.92 (s, 1 H) 8.40 (s, 1 H) 9.50 (s, 1 H); HRMS (ESI+) calcd for C₂₃H₁₈Cl₂FN₇S (MH+) 514.07782, found 514.0785.

### Example 281

### 8-Chloro-4-[(3-chloro-4-fluorophenyl)amino]-6-({[2-(methylthio)pyrimidin-5-yl]methyl]amino)quinoline-3-carbonitrile

Following the procedure described above in Example 4, 6-amino-4-[(3-chloro-4-fluorophenyl)amino]-8-chloroquinoline-3-carbonitrile (70 mg, 0.20 mmol) was reacted with 2-methylsulfanyl-pyrimidine-5-carbaldehyde (50.0 mg, 0.32 mmol) and NaCNBH₃ (8.8 mg, 0.14 mmol) in 3 mL EtOH. The crude product was purified by preparative HPLC, and lyophilized to give the product as a yellow solid (36.8 mg, 38%): 1H NMR (400 MHz, DMSO-D6) δ ppm 2.50-2.54 (m, 3 H) 4.37 (d, J=5.56 Hz, 2 H) 6.98 (t, J=5.68 Hz, 1 H) 7.23 (d, J=2.27 Hz, 1 H) 7.26-7.33 (m, 1 H) 7.45 (t, J=8.97 Hz, 1 H) 7.49-7.58 (m, 2 H) 8.41 (s, 1 H) 8.68 (s, 2 H) 9.47 (s, 1 H); HRMS (ESI+) calcd for C₂₂H₁₅Cl₂FN₆S (MH+) 485.05127, found 485.0532.

### Example 282

### 8-Chloro-4-[(3-chloro-4-fluorophenyl)amino]-6-({[2-(methylsulfonyl)pyrimidin-4-yl]methyl}amino)quinoline-3-carbonitrile

Following the procedure described above in Example 4, 6-amino-4-[(3-chloro-4-fluorophenyl)amino]-8-chloroquinoline-3-carbonitrile (40 mg, 0.12 mmol) was reacted with 2-methanesulfonyl-pyrimidine-4-carbaldehyde (22.0 mg, 0.13 mmol) and NaCNBH₃ (5.3 mg, 0.08 mmol) in 3 mL EtOH. The crude product was purified by preparative HPLC, and lyophilized to give the product as a yellow solid (2.3 mg, 4%): 1H NMR (400 MHz, DMSO-D6) δ ppm 3.39 (s, 3 H) 4.73 (d, J=6.32 Hz, 2 H) 6.55 (s, 1 H) 7.20-7.30 (m, 3 H) 7.43 (t, J=8.97 Hz, 1 H) 7.51 (dd, J=6.57, 2.53 Hz, 1 H) 7.66 (d, J=2.53 Hz, 1 H) 7.72 (d, J=5.31 Hz, 1 H) 8.39-8.42 (m, 1 H) 8.99 (d, J=5.31 Hz, 1 H) 9.44 (s, 1 H); HRMS (ESI+) calcd for C₂₂H₁₅Cl₂FN₆O₂S (MH+) 517.04110, found 517.0427.

### Example 283

### 8-chloro-4-[(3-chloro-4-fluorophenyl)amino]-6-{[2-(1H-imidazol-5-yl)ethyl]amino}quinoline-3-carbonitrile

Step 1: In a microwave vial, triphenyl phosphine (0.50 g, 1.91 mmol) was taken up in 4 mL toluene, and bromo methoxy methane (0.29 g, 2.29 mmol) was added. The vial was crimp-sealed and heated in a microwave reactor at 140° C. for 5 minutes, until LC-MS analysis showed complete disappearance of starting material. The content of the vial was transferred to a round-bottomed flask and evaporated solvent in vacuo. The solid obtained was suspended in toluene, then filtered, washed with toluene to give the product methoxymethyl-triphenyl-phosphonium; bromide as a white solid (0.60 g, 81% yield): 1H NMR (400 MHz, DMSO-D6) δ ppm 3.48 (s, 3 H) 5.63 (d, J=5.05 Hz, 2 H) 7.70-7.86 (m, 15 H).

Step 2: In a microwave vial, the product from the previous step (300.0 mg, 0.77 mmol) was taken up in 4 mL THF, and NaH (46.5 g, 0.46 mmol, 60% in mineral oil) was added to this suspension. The vial was crimp-sealed and heated in a microwave reactor at 80° C. for 5 minutes, then trityl imidazole aldehyde (104.9 mg, 0.31 mmol) was added to the reaction mixture and stirred at RT for 3 hours, until LC-MS analysis showed complete disappearance of starting material. The solvent was evaporated and the crude product was purified by flash chromatography over silica gel (5% MeOH in CH₂Cl₂) to give the product 5-(2-methoxy-vinyl)-1-trityl-1H-imidazole as a white solid (90.0 mg, 79% yield).

Step 3: In a 50 mL round-bottomed flask equipped with a condenser, the product from step 3 (90 mg, 0.25 mmol) was taken up in 5 mL 1 N HCl and 3 mL THF and heated at 60° C. for 2 hours under nitrogen, until LC-MS analysis showed complete disappearance of starting material. The reaction mixture was then allowed to cool to RT and evaporated solvent in vacuo to give a white solid as product (3H-imidazol-4-yl)-acetaldehyde of sufficient purity to be used in the next step (26 mg, 96% yield).

Step 4: Following the procedure described above in Example 4, 6-amino-4-[(3-chloro-4-fluorophenyl)amino]-8-chloroquinoline-3-carbonitrile (85 mg, 0.24 mmol) was reacted with (3H-imidazol-4-yl)-acetaldehyde (26.0 mg, 0.24 mmol) and NaCNBH₃ (13.8 mg, 0.22 mmol) in 5 mL EtOH. The crude product was purified by preparative HPLC, and lyophilized to give the product as a yellow solid (15.1 mg, 14%): 1H NMR (400 MHz, DMSO-D6) δ ppm 2.83 (t, J=7.07 Hz, 2 H) 3.31-3.44 (m, 2 H) 6.56 (t, J=5.68 Hz, 1 H) 6.87 (s, 1 H) 7.12 (d, J=2.27 Hz, 1 H) 7.24-7.35 (m, 1 H) 7.45 (t, J=9.09 Hz, 1 H) 7.49 (d, J=2.27 Hz, 1 H) 7.51-7.60 (m, 2 H) 8.20 (s, 1 H) 8.37 (s, 1 H) 9.53 (s, 1 H); HRMS (ESI-) calcd for C₂₁H₁₅Cl₂FN₆ (MH−) 439.06465, found 439.0661.

### Example 284

### 4-[(3-chloro-4-fluorophenyl)amino]-6-[(1H-imidazol-5-ylmethyl)amino]-8-(methylsulfinyl)quinoline-3-carbonitrile

Step 1: In a 50 mL round-bottomed flask, 8-fluoro-4-hydroxy-6-nitro-quinoline-3-carbonitrile (0.50 g, 2.14 mmol) was taken up in 9 mL DMPU. MeSNa (0.57 g, 8.1 mmol) was added. The reaction mixture was stirred at RT overnight, and then the mixture was poured into ice-water, 1 N HCl was added slowly until pH=6. Lots of precipitate formed. The mixture was filtered, washed with water to give a black solid. The crude product was purified by preparative HPLC, and lyophilized to give the product 8-(methylthio)-6-nitro-4-oxo-1,4-dihydroquinoline-3-carbonitrile as a yellow solid (0.21 g, 38%): 1H NMR (400 MHz, DMSO-D6) δ ppm 2.72 (s, 3 H) 8.39 (d, J=2.27 Hz, 1 H) 8.54-8.76 (m, 2 H) 12.45 (s, 1 H).

Step 2: In a 100 mL round-bottomed flask equipped with a condenser, 4-hydroxy-8-methylsulfanyl-6-nitro-quinoline-3-carbonitrile (0.19 g, 0.71 mmol) was taken up in 6 mL POCl₃ and heated at reflux for 5 hours. The reaction mixture was then stirred at RT overnight, and then POCl₃ was removed under reduced pressure. Ice chips were added to the residue and then saturated NaHCO₃ solution was added carefully, the mixture was stirred for 30 minutes, checking the pH periodically to ensure that it remained at or above 8. The mixture was filtered and dried under high vacuum overnight to give the product 4-chloro-8-methylsulfanyl-6-nitro-quinoline-3-carbonitrile as a brown solid (0.18 g, 90% yield): 1H NMR (400 MHz, DMSO-D6) δ ppm 2.68 (s, 3 H) 8.21 (s, 1 H) 8.71 (s, 1 H) 9.40 (s, 1 H).

Step 3: Following the procedure described above in Example 229, 4-chloro-8-methylsulfanyl-6-nitro-quinoline-3-carbonitrile (0.18 g, 0.64 mmol) was reacted with 3-chloro-4-fluoroaniline (0.11 g, 0.77 mmol) in 4 mL EtOH. After work up, 4-[(3-chloro-4-fluorophenyl)amino]-8-(methylthio)-6-nitroquinoline-3-carbonitrile was obtained as a yellow solid (0.18 g, 72% yield): 1H NMR (400 MHz, DMSO-D6) δ ppm 2.60 (s, 3 H) 7.41-7.48 (m, 1 H) 7.53 (t, J=8.97 Hz, 1 H) 7.71 (dd, J=6.69, 2.40 Hz, 1 H) 8.11 (d, J=2.02 Hz, 1 H) 8.78 (s, 1 H) 9.26 (d, J=2.27 Hz, 1 H) 10.52 (s, 1 H).

Step 4: To a solution of the product from step 3 (50.0 mg, 0.13 mmol) in 2 mL CH₂Cl₂ was added a solution of mcPBA (28.8 mg, 0.13 mmol) in 2 mL CH₂Cl₂ slowly through an additional funnel at −5° C. The reaction mixture was then stirred at −5° C.-0° C. for 1.5 hours, until TLC analysis showed complete disappearance of starting material. 5 mL saturated NaHCO3 solution was added to the reaction mixture at 0° C. and the layers were separated. The organic layer was washed with saturated NaHCO₃ solution and brine, separated, and concentrated to yield 4-(3-chloro-4-fluoro-phenylamino)-8-methanesulfinyl-6-nitro-quinoline-3-carbonitrile as a yellow solid (56.0 mg, 100% yield), which was used in the next step directly without further purification: 1H NMR (400 MHz, DMSO-D6) δ ppm 2.95 (s, 3 H) 7.38 (s, 1 H) 7.46-7.57 (m, 1 H) 7.65 (s, 1 H) 8.73 (d, J=2.27 Hz, 2 H) 9.65 (d, J=3.03 Hz, 1 H) 10.82(s, 1 H).

Step 5: Following the procedure described above in Example 229, 4-(3-chloro-4-fluoro-phenylamino)-8-methanesulfinyl-6-nitro-quinoline-3-carbonitrile (56.0 mg, 0.14 mmol) was reacted with tin chloride dihydrate (0.13 g, 0.55 mmol) in 4 mL EtOH. After work up, product 6-amino-4-(3-chloro-4-fluoro-phenylamino)-8-methanesulfinyl-quinoline-3-carbonitrile was obtained as a light brown solid (37.0 mg, 71% yield): 1H NMR (400 MHz, DMSO-D6) δ ppm 2.86 (s, 3 H) 6.16 (s, 2 H) 7.20-7.30 (m, 2 H) 7.40-7.50 (m, 2 H) 7.69 (d, J=2.27 Hz, 1 H) 8.35 (s, 1 H) 9.60 (s, 1 H).

Step 6: Following the procedure described above in Example 4, 6-amino-4-(3-chloro-4-fluoro-phenylamino)-8-methanesulfinyl-quinoline-3-carbonitrile (35.0 mg, 0.09 mmol) was reacted with 4(5)-imidazolecarboxyaldehyde (16.2 mg, 0.17 mmol) and NaCNBH₃ (4.0 mg, 0.06 mmol) in 3 mL EtOH. The crude product was purified by preparative HPLC, and lyophilized to give the product as a yellow solid (10.0 mg, 24%). HRMS (ESI−) calcd for C₂₁H₁₆ClFN₆OS (M−H+) 453.07061, found 453.0726:

1H NMR (400 MHz, DMSO-D6) δ ppm 2.86 (s, 3 H) 4.32 (d, J=5.31 Hz, 2 H) 7.01-7.10 (m, 2 H) 7.29-7.37 (m, 2 H) 7.42-7.51 (m, 1 H) 7.55-7.63 (m, 2 H) 7.81 (d, J=2.27 Hz, 1 H) 8.23 (s, 1 H) 8.32 (s, 1 H) 9.60 (s, 1 H).

### Example 285

### 8-Chloro-4-[(3-chloro-4-fluorophenyl)amino]-6-{[(1-methyl-2H-tetrazol-5-yl)methyl]amino}quinoline-3-carbonitrile and 8-Chloro-4-[(3-chloro-4-fluorophenyl)amino]-6-{[(2-methyl-2H-tetrazol-5-yl)methyl]amino}quinoline-3-carbonitrile

Step 1: In a pressure tube, 5-diethoxymethyl-1H-tetrazole (0.4 g, 2.32 mmol) was taken up in 10 mL THF. MeI (0.66 g, 465 mmol) and K₂CO₃ (0.64 g, 4.65 mmol) were added. The tube was capped and heated at 50° C. overnight. The reaction mixture was allowed to cool to RT, then it was filtered, washed with THF, and concentrated to a dark brown liquid. 10 mL of 1.25M HCl in MeOH was added to the residue, the mixture was heated at reflux for 5 hours, then allowed to cool down to RT and evaporated down solvent to give a dark brown oil (˜0.3 g). The crude product was used in the next step directly without further purification.

Step 2: Following the procedure described above in Example 4, 6-amino-4-[(3-chloro-4-fluorophenyl)amino]-8-chloroquinoline-3-carbonitrile (100 mg, 0.29 mmol) was reacted with a mixture of 1-methyl-1H-tetrazole-5-carbaldehyde and 2-methyl-2H-tetrazole-5-carbaldehyde (260 mg, 2.32 mmol) and NaCNBH₃ (12.8 mg, 0.20 mmol) in 5 mL EtOH. The crude product was purified by preparative HPLC, and lyophilized to give the 2-methyl product as a yellow solid (4.5 mg, 7%): 1H NMR (400 MHz, DMSO-D6) δ ppm 4.33 (s, 3 H) 4.69 (d, J=6.06 Hz, 2 H) 7.05 (s, 1 H) 7.25 (s, 1 H) 7.33 (s, 1 H) 7.43 (t, J=9.35 Hz, 1 H) 7.49 (s, 1 H) 7.57 (s, 1 H) 8.36 (s, 1 H) 8.44 (s, 1 H); and the 1-methyl product as a yellow solid (1.5 mg, 2.3%): 1H NMR (400 MHz, DMSO-D6) δ ppm 4.08 (s, 3 H) 4.77 (d, J=5.81 Hz, 2 H) 7.06 (s, 1 H) 7.21-7.28 (m, 1 H) 7.36 (d, J=2.02 Hz, 1 H) 7.42 (t, J=8.97 Hz, 1 H) 7.48 (d, J=4.55 Hz, 1 H) 7.56 (s, 1 H) 8.38 (s, 1 H) 8.44 (s, 1 H); HRMS (ESI+) calcd for C₁₉H₁₃Cl₂FN₈ (MH+) 443.06970, found 443.0697.

### Example 286

### 4-[(3-chloro-4-fluorophenyl)amino]-6-[(1H-imidazol-5-ylmethyl)amino]-8-(methylsulfonyl)quinoline-3-carbonitrile

Step 1: To a solution of 4-(3-chloro-4-fluoro-phenylamino)-8-methylsulfanyl-6-nitro-quinoline-3-carbonitrile (70.0 mg, 0.18 mmol) in 2 mL THF was added a solution of mcPBA (100.9 mg, 0.45 mmol) in 3 mL THF slowly through an additional funnel at 0° C. The reaction mixture was then stirred at 0° C. for 30 minutes, then it was allowed to warm to RT and stirred at RT for 2 days until TLC analysis showed complete disappearance of starting material. 5 mL saturated NaHCO₃ solution was added to the reaction mixture at 0° C., then 15 mL of EtOAC was added. The layers were separated, the organic layer was washed with saturated NaHCO₃ and brine, and concentrated to give 4-(3-chloro-4-fluoro-phenylamino)-8-methanesulfonyl-6-nitro-quinoline-3-carbonitrile as a light brown solid (70 mg, 93% yield). The product was used in the next step directly without further purification: 1H NMR (400 MHz, DMSO-D6) δ ppm 3.47-3.61 (m, 3 H) 7.45-7.53 (m, 2 H) 7.56-7.67 (m, 2 H) 7.86-7.89 (m, 3H).

Step 2: Following the procedure described above in Example 229, 4-(3-chloro-4-fluoro-phenylamino)-8-methanesulfonyl-6-nitro-quinoline-3-carbonitrile (70 mg, 0.17 mmol) was reacted with tin chloride dihydrate (0.23 g, 1.04 mmol) in 4 mL EtOH. After work up, product 6-amino-4-(3-chloro-4-fluoro-phenylamino)-8-methanesulfonyl-quinoline-3-carbonitrile was isolated as a dark yellow solid (50.0 mg, 77% yield).

Step 3: Following the procedure described above in Example 4, 6-amino-4-(3-chloro-4-fluoro-phenylamino)-8-methanesulfonyl-quinoline-3-carbonitrile (50.0 mg, 0.12 mmol) was reacted with 4(5)-imidazolecarboxyaldehyde (20.8 mg, 0.22 mmol) and NaCNBH₃ (5.2 mg, 0.08 mmol) in 3 mL EtOH. The crude product was purified by preparative HPLC, and lyophilized to give the product as a yellow solid (9.0 mg, 15%): 1H NMR (400 MHz, DMSO-D6) δ ppm 3.52 (s, 3 H) 4.32 (d, J=5.05 Hz, 2 H) 7.04-7.08 (m, 1 H) 7.11 (t, J=5.81 Hz, 1 H) 7.31-7.37 (m, 1 H) 7.47 (t, J=8.97 Hz, 1 H) 7.51 (d, J=2.27 Hz, 1 H) 7.58 (dd, J=6.57, 2.53 Hz, 1 H) 7.61 (d, J=1.01 Hz, 1 H) 8.12 (d, J=2.27 Hz, 1 H) 8.28 (s, 1 H) 8.45 (s, 1 H); HRMS (ESI+) calcd for C₂₁H₁₆ClFN₆O₂S (MH+) 471.08007, found 471.0796.

### Example 287

### 4-[(3-chloro-4-fluorophenyl)amino]-6-[(1H-imidazol-5-ylmethyl)amino]-8-(methylthio)quinoline-3-carbonitrile

Step 1: Following the procedure described above in Example 229, 4-(3-chloro-4-fluoro-phenylamino)-8-methylsulfanyl-6-nitro-quinoline-3-carbonitrile (54.0 mg, 0.14 mmol) was reacted with tin chloride dihydrate (0.13 g, 0.56 mmol) in 3 mL EtOH. After work up, product 6-amino-4-[(3-chloro-4-fluorophenyl)amino]-8-(methylthio)quinoline-3-carbonitrile was obtained as a dark yellow solid (45.8 mg, 91% yield): 1H NMR (400 MHz, DMSO-D6) δ ppm 2.42 (s, 3 H) 5.80 (s, 2 H) 6.88 (d, J=2.02 Hz, 1 H) 7.01 (d, J=2.02 Hz, 1 H) 7.13-7.16 (m, 1 H) 7.34-7.40 (m, 2 H) 8.33 (s, 1 H) 9.34 (s, 1 H).

Step 2: Following the procedure described above in Example 4, 6-amino-4-(3-chloro-4-fluoro-phenylamino)-8-methylsulfanyl-quinoline-3-carbonitrile (44.0 mg, 0.12 mmol) was reacted with 4(5)-imidazolecarboxyaldehyde (21.2 mg, 0.22 mmol) and NaCNBH₃ (5.3 mg, 0.08 mmol) in 3 mL EtOH. The crude product was purified by preparative HPLC, and lyophilized to give the product as a yellow solid (28.0 mg, 53%): 1H NMR (400 MHz, DMSO-D6) δ ppm 2.39 (s, 3 H) 4.25 (d, J=5.05 Hz, 2 H) 6.48 (d, 1 H) 6.95 (d, J=1.77 Hz, 1 H) 7.05 (s, 1 H) 7.18 (d, J=2.02 Hz, 1 H) 7.22-7.28 (m, 1 H) 7.38-7.51 (m, 2 H) 7.62 (d, J=1.01 Hz, 1 H) 8.19 (s, 1 H) 8.30 (s, 1 H) 9.34 (s, 1 H); HRMS (ESI+) calcd for C₂₁H₁₆ClFN₆S (MH+) 439.09024, found 439.0898.

### Example 288

### 8-Bromo-4-(3-chloro-4-fluoro-phenylamino)-6-[(1-phenyl-1H-[1,2,3]triazol-4-ylmethyl)-amino]-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and 1-Phenyl-1H-[1,2,3]triazole-4-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT for 24 h. The reaction mixture was stripped to dryness and the residue was purified via preparative HPLC, and lyophilized to give the product as a yellow solid (0.062 g, 76%). 1H NMR (400 MHz, DMSO-D6) δ ppm 4.56 (s, 2 H) 7.26-7.34 (m, 2 H) 7.44 (t, J=9.22 Hz, 1 H) 7.51 (d, J=6.06 Hz, 2 H) 7.59 (t, J=7.58 Hz, 2 H) 7.76-7.85 (m, 3 H) 8.39 (s, 1 H) 8.67 (s, 1 H); HRMS (ESI+) calcd for C₂₅H₁₆BrClFN₇ (MH+) 548.03958, found 548.0406.

### Example 289

### 8-Bromo-4-(3-chloro-4-fluoro-phenylamino)-6-{[1-(4-methoxy-phenyl)-1H-[1,2,3]triazol-4-ylmethyl]-amino}-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and 1-(4-methoxy-phenyl)-1H-[1,2,3]triazole-4-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT overnight. The reaction mixture was stripped to dryness and the residue was purified via preparative HPLC, and lyophilized to give the product as a yellow solid (0.056 g, 64%). 1H NMR (400 MHz, DMSO-D6) δ ppm 3.72 (s, 3 H) 4.44 (s, 2 H) 5.46 (s, 2 H) 6.86 (d, J=8.59 Hz, 2 H) 7.25 (t, J=8.97 Hz, 4 H) 7.41-7.52 (m, 2 H) 7.73 (d, J=1.52 Hz, 1 H) 8.01 (s, 1 H) 8.37 (s, 1 H).

### Example 290

### 8-Bromo-4-(3-chloro-4-fluoro-phenylamino)-6-[(5-phenyl-2H-[1,2,4]triazol-3-ylmethyl)-amino]-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and 5-Phenyl-2H-[1,2,4]triazole-3-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT for 0.5 h, then the reaction was proceeded at 50° C. overnight. The reaction mixture was stripped to dryness and the residue was purified via preparative HPLC, and lyophilized to give the product as a yellow solid (0.041 g, 51%). 1H NMR (400 MHz, DMSO-D6) δ ppm 4.56 (s, 2 H) 7.25-7.30 (m, 1 H) 7.34 (d, J=2.27 Hz, 1 H) 7.40-7.52 (m, 5 H) 7.81 (d, J=1.77 Hz, 1 H) 7.95 (dd, J=7.20, 2.15 Hz, 2 H) 8.40 (s, 1 H).

### Example 291

### (4-{[8-Bromo-4-(3-chloro-4-fluoro-phenylamino)-3-cyano-quinolin-6-ylamino]-methyl}-[1,2,3]triazol-1-yl)-acetic acid

In a 15 mL round-bottomed flask were added 6-amino-4-(4-bromo-3-fluoro-phenylamino)-quinoline-3-carbonitrile (0.06 g, 0.15 mmol), ethanol (4 mL) and (4-Formyl-[1,2,3]triazol-1-yl)-acetic acid ethyl ester (0.4 mmol). Acetic acid was added to bring the pH of the solution to 4, and the mixture was stirred for 15 minutes. Sodium triacetoxyborohydride (0.5 mmol) was then added and the reaction was stirred at RT overnight. The reaction mixture was diluted with water and the precipitate was collected via filtration. The crude was treated with a solution of LiOH (1 mmol) in THF-water (1:1, 3 mL) for 4 h. The reaction was acidified with dilute HCl to bring the pH to 4. The precipitate was collected through filtration to give pure product 40 mg (yield 51%). 1H NMR (400 MHz, DMSO-D6) δ ppm 4.38 (d, J=4.04 Hz, 2 H) 4.55 (s, 2 H) 6.72 (s, 1 H) 7.19 (s, 1 H) 7.33 (s, 3 H) 7.68 (s, 1 H) 7.83-7.90 (m, 1 H) 8.24 (s, 1 H).

### Example 292

### 4-(4-{[8-Bromo-4-(3-chloro-4-fluoro-phenylamino)-3-cyano-quinolin-6-ylamino]-methyl}-[1,2,3]triazol-1-yl)-benzoic acid

In a 15 mL round-bottomed flask were added 6-amino-4-(4-bromo-3-fluoro-phenylamino)-quinoline-3-carbonitrile (0.06 g, 0.15 mmol), ethanol (4 mL) and 4-(4-Formyl-[1,2,3]triazol-1-yl)-benzoic acid (0.4 mmol). Acetic acid was added to bring the pH of the solution to 4, and the mixture was stirred for 15 minutes. Sodium triacetoxyborohydride (0.5 mmol) was then added and the reaction was stirred at RT overnight. The reaction mixture was stripped to dryness and the residue was purified via preparative HPLC, and lyophilized to give the product as a yellow solid (0.035 g, 41%). 1H NMR (400 MHz, MeOD) δ ppm 4.62 (s, 2 H) 7.23-7.27 (m, 2 H) 7.41 (s, 1 H) 7.74 (d, J=2.27 Hz, 1 H) 7.92 (d, J=8.84 Hz, 1 H) 8.17 (d, J=9.09 Hz, 2 H) 8.30-8.40 (m, 3 H) 8.56 (s, 1 H).

### Example 293

### 8-Bromo-4-(3-chloro-4-fluoro-phenylamino)-6-[(5-diethoxymethyl-1H-[1,2,3]triazol-4-ylmethyl)-amino]-quinoline-3-carbonitrile

Step 1: 4,4-Diethoxy-but-2-ynal (0.78 g, 5 mmol) was added to a solution of sodium azide (10 mmol) in DMSO (5 mL) cooled with an ice bath. After 1 h, the reaction was diluted with ethyl acetate (25 mL) and water (10 mL) and pH was adjusted to 7 with dilute HCl. The two layers were separated and the aqueous layer was extracted with ethyl acetate (15 mL) twice. The combined organic layers were washed with brine and dried over sodium sulfate. Evaporation of the solvent provided 0.74 g of 5-diethoxymethyl-1H-[1,2,3]triazole-4-carbaldehyde (yield 74%).

1H NMR (400 MHz, chloroform-D) δ ppm 1.16-1.33 (m, 6 H) 3.63-3.83 (m, 4 H) 6.05 (s, 1 H) 10.27 (s, 1 H).

Step 2: In a 15 mL round-bottomed flask were added 6-amino-4-(4-bromo-3-fluoro-phenylamino)-quinoline-3-carbonitrile (0.26 g, 0.6 mmol), ethanol (10 mL) and 5-Diethoxymethyl-1H-[1,2,3]triazole-4-carbaldehyde (1.8 mmol). Acetic acid was added to bring the pH of the solution to 4, and the mixture was stirred for 15 minutes. Sodium triacetoxyborohydride (1.8 mmol) was then added and the reaction was stirred at RT for 72 h. The reaction mixture was stripped to dryness and the residue was purified via preparative HPLC, and lyophilized to give the product as a yellow solid (0.15 g, 44%). 1H NMR (400 MHz, MeOD) δ ppm 1.18 (t, J=7.07 Hz, 6 H) 3.54-3.71 (m, 4 H) 4.56 (s, 2 H) 5.78 (s, 1 H) 7.19-7.31 (m, 3 H) 7.41 (dd, J=6.82, 2.53 Hz, 1 H) 7.70 (d, J=2.27 Hz, 1 H) 8.32 (s, 1 H).

### Example 294

### 8-Bromo-4-(3-chloro-4-fluoro-phenylamino)-6-[(5-hydroxymethyl-1H-[1,2,3]triazol-4-ylmethyl)-amino]-quinoline-3-carbonitrile

8-Bromo-4-(3-chloro-4-fluoro-phenylamino)-6-[(5-diethoxymethyl-1H-[1,2,3]triazol-4-ylmethyl)-amino]-quinoline-3-carbonitrile (30 mg) was dissolved into methanol (3 mL). Hydrochloric acid (3 M, 1 mL) was added to the solution and the solution was heated to 60° C. for 1 h. The solution was cooled with an ice bath and neutralized with sodium carbonate solution to pH=5. The resulting precipitate was filtered and collected to give the crude aldehyde product. To the crude was added methanol (3 mL), and sodium borohydride (20 mg) and the mixture was stirred at RT for 5 h. The product was purified by HPLC to give product (26 mg, 95%). 1H NMR (400 MHz, MeOD) δ ppm 4.52 (s, 2 H) 4.75 (s, 2 H) 7.23-7.34 (m, 3 H) 7.44 (dd, J=6.44, 1.89 Hz, 1 H) 7.68 (d, J=2.02 Hz, 1 H) 8.26 (s, 1 H).

### Example 295

### 8-Bromo-4-(3-chloro-4-fluoro-phenylamino)-6-({5-[(2-hydroxy-ethylamino)-methyl]-1H-[1,2,3]triazol-4-ylmethyl}-amino)-quinoline-3-carbonitrile

8-Bromo-4-(3-chloro-4-fluoro-phenylamino)-6-[(5-diethoxymethyl-1H-[1,2,3]triazol-4-ylmethyl)-amino]-quinoline-3-carbonitrile (30 mg) was dissolved in methanol (3 mL). Hydrochloric acid (3M, 1 mL) was added to the solution and the solution was heated to 60° C. for 1 h. The solution was cooled with an ice bath and neutralized with sodium carbonate solution to pH=5. The resulting precipitate was filtered and collected to give the crude aldehyde product. In a 15 mL round-bottomed flask were added the crude product, ethanol (4 mL) and 2-aminoethanol (0.1 mmol). Acetic acid was added to bring the pH of the solution to 4, and the mixture was stirred for 15 minutes. Sodium triacetoxyborohydride (0.5 mmol) was then added and the reaction was stirred at RT for 4 h. The reaction mixture was stripped to dryness and the residue was purified via preparative HPLC and lyophilized to give the product as a yellow solid (0.026 g, 92%). 1H NMR (400 MHz, MeOD) δ ppm 2.81-2.90 (m, 2 H) 3.64-3.75 (m, 2 H) 4.06 (s, 2 H) 4.55 (s, 2 H) 7.21-7.31 (m, 3 H) 7.42 (dd, J=6.19, 2.40 Hz, 1 H) 7.71 (d, J=2.53 Hz, 1 H) 8.32 (s, 1 H).

### Example 296

### 8-Bromo-4-(3-chloro-4-fluoro-phenylamino)-6-{[1-(2-piperidin-1-yl-ethyl)-1H-[1,2,3]triazol-4-ylmethyl]-amino}-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and 1-(2-piperidin-1-yl-ethyl)-1H-[1,2,3]triazole-4-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT for 96 h. The reaction mixture was stripped to dryness and the residue was purified via preparative HPLC, and lyophilized to give the product as a yellow solid (0.04 g, 46%). 1H NMR (400 MHz, MeOD) δ ppm 1.30-1.49 (m, 6 H) 2.37 (s, 4 H) 2.74 (t, J=6.44 Hz, 2 H) 4.48 (t, J=6.44 Hz, 2 H) 4.54 (s, 2 H) 7.17 (d, J=2.53 Hz, 1 H) 7.21-7.25 (m, 1 H) 7.28 (t, J=8.72 Hz, 1 H) 7.40 (dd, J=6.69, 2.40 Hz, 1 H) 7.69 (d, J=2.53 Hz, 1 H) 7.91 (s, 1 H) 8.30 (s, 1 H); HRMS (ESI+) calcd for C₂₆H₂₅BrClFN₈ (MH+) 583.11308, found 583.113.

### Example 297

### 8-Bromo-4-(3-chloro-4-fluoro-phenylamino)-6-{[1-(2-morpholin-4-yl-ethyl)-1H-[1,2,3]triazol-4-ylmethyl]-amino}-quinoline-3-carbonitrile

In a 50 mL round-bottomed flask were added 6-amino-4-(4-bromo-3-fluoro-phenylamino)-quinoline-3-carbonitrile (0.90 g, 2.30 mmol), dichloroethane (15 mL) and 1-(2-morpholin-4-yl-ethyl)-1H-[1,2,3]triazole-4-carbaldehyde (2.5 mmol). Acetic acid was added to bring the pH of the solution to 4, and the mixture was stirred for 15 minutes. Sodium triacetoxyborohydride (0.5 mmol) was then added and the reaction was stirred at RT for 5 h. The reaction mixture was stripped to dryness and the residue was purified via preparative HPLC, and lyophilized to give the product as a yellow solid (0.72 g, 71%). 1H NMR (400 MHz, MeOD) δ ppm 2.80-2.89 (m, 4 H) 3.17-3.23 (m, J=6.06, 6.06 Hz, 2 H) 3.89-3.95 (m, 4 H) 4.87-4.95 (m, 4 H) 7.55-7.58 (m, J=2.27 Hz, 1 H) 7.60-7.64 (m, 1 H) 7.68 (t, J=8.84 Hz, 1 H) 7.80 (dd, J=6.57, 2.53 Hz, 1 H) 8.08 (d, J=2.53 Hz, 1 H) 8.32 (s, 1 H) 8.48 (s, 1 H) 8.70 (s, 1 H); HRMS (ESI+) calcd for C₂₅H₂₃BrClFN₈O (MH+) 585.09235, found 585.0921.

### Example 298

### 8-Bromo-4-(3-chloro-4-fluoro-phenylamino)-6-{[1-(2-morpholin-4-yl-2-oxo-ethyl)-1H-[1,2,3]triazol-4-ylmethyl]-amino}-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), dichloroethane (2 mL) and 1-(2-morpholin-4-yl-2-oxo-ethyl)-1H-[1,2,3]triazole-4-carbaldehyde (0.22 mmol). The mixture was stirred for 15 minutes. Sodium triacetoxyborohydride (0.45 mmol) was then added and the reaction was stirred at RT for 5 h. The reaction mixture was stripped to dryness and the residue was purified via preparative HPLC and lyophilized to give the product as a yellow solid (0.079 g, 89%). 1H NMR (400 MHz, MeOD) δ ppm 1.12-1.23 (m, 2 H) 2.08 (d, J=1.52 Hz, 2 H) 2.70-2.71 (m, 2 H) 3.52-3.84 (m, 4 H) 4.57 (s, 2 H) 7.29 (s, 4 H) 7.43 (s, 1 H) 7.69 (s, 1 H) 8.30 (d, J=3.03 Hz, 1 H).

### Example 299

### 8-Bromo-4-(3-chloro-4-fluoro-phenylamino)-6-[(furan-3-ylmethyl)-amino]-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and furan-3-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT overnight. The reaction mixture was stripped to dryness and the residue was purified via preparative HPLC, and lyophilized to give the product as a yellow solid (0.035 g, 50%). 1H NMR (400 MHz, MeOD) δ ppm 4.24 (s, 2 H) 6.45 (s, 1 H) 7.12 (d, J=2.02 Hz, 1 H) 7.19-7.23 (m, 1 H) 7.27 (t, J=8.72 Hz, 1 H) 7.39 (dd, J=6.44, 2.40 Hz, 1 H) 7.44-7.50 (m, 2 H) 7.68 (d, J=2.02 Hz, 1 H) 8.29 (s, 1 H); HRMS (ESI+) calcd for C₂₁H₁₃BrClFN₄O (MH+) 471.00180, found 471.0004.

### Example 300

### 8-Chloro-4-(3-chloro-4-fluoro-phenylamino)-6-{[1-(2-piperidin-1-yl-ethyl)-1H-[1,2,3]triazol-4-ylmethyl]-amino}-quinoline-3-carbonitrile

In a 15 mL round-bottomed flask were added 6-amino-4-(3-chloro-4-fluoro-phenylamino)-quinoline-3-carbonitrile (0.052 g, 0.15 mmol), dichloroethane (2 mL) and 1-(2-Piperidin-1-yl-ethyl)-1H-[1,2,3]triazole-4-carbaldehyde (0.22 mmol). The mixture was stirred for 15 minutes. Sodium triacetoxyborohydride (0.45 mmol) was then added and the reaction was stirred at RT for 5 h. The reaction mixture was stripped to dryness and the residue was purified via preparative HPLC, and lyophilized to give the product as a yellow solid (0.056 g, 70%). 1H NMR (400 MHz, MeOD) δ ppm 1.66-1.88 (m, 6 H) 2.96 (s, 4 H) 3.30-3.39 (m, 2 H) 4.77 (s, 2 H) 4.85 (t, J=6.44 Hz, 2 H) 7.40 (t, J=2.53 Hz, 1 H) 7.42-7.47 (m, 1 H) 7.50 (t, J=8.84 Hz, 1 H) 7.62 (dd, J=6.57, 2.53 Hz, 1 H) 7.90 (d, J=2.27 Hz, 1 H) 8.16 (s, 1 H) 8.48 (s, 1 H) 8.52 (s, 1 H); HRMS (ESI+) calcd for C₂₆H₂₅Cl₂FN₈ (MH+) 539.16360, found 539.1623.

### Example 301

### 8-Chloro-4-(3-chloro-4-fluoro-phenylamino)-6-{[1-(2-morpholin-4-yl-ethyl)-1H-[1,2,3]triazol-4-ylmethyl]-amino}-quinoline-3-carbonitrile

In a 15 mL round-bottomed flask were added 6-amino-4-(3-chloro-4-fluoro-phenylamino)-quinoline-3-carbonitrile (0.052 g, 0.15 mmol), dichloroethane (2 mL) and 1-(2-Morpholin-4-yl-ethyl)-1H-[1,2,3]triazole-4-carbaldehyde (0.22 mmol). The mixture was stirred for 15 minutes. Sodium triacetoxyborohydride (0.45 mmol) was then added and the reaction was stirred at RT for 5 h. The reaction mixture was stripped to dryness and the residue was purified via preparative HPLC, and lyophilized to give the product as a yellow solid (0.047 g, 59%). 1H NMR (400 MHz, MeOD) δ ppm 2.39-2.52 (m, 4 H) 2.83 (t, J=6.19 Hz, 2 H) 3.49-3.60 (m, 4 H) 4.44-4.58 (m, 4 H) 7.13-7.18 (m, J=2.53 Hz, 1 H) 7.23-7.33 (m, 2 H) 7.42 (dd, J=6.44, 2.40 Hz, 1 H) 7.49 (d, J=2.27 Hz, 1 H) 7.94 (s, 1 H) 8.09 (s, 2 H) 8.31 (s, 1 H); HRMS (ESI+) calcd for C₂₅H₂₃Cl₂FN₈O (MH+) 541.14287, found 541.1424.

### Example 302

### 8-Bromo-4-(3-chloro-4-fluoro-phenylamino)-6-[([1,2,3]thiadiazol-4-ylmethyl)-amino]-quinoline-3-carbonitrile

In a 15 mL round-bottomed flask were added 6-amino-4-(4-bromo-3-fluoro-phenylamino)-quinoline-3-carbonitrile (0.06 g, 0.15 mmol), ethanol (4 mL) and [1,2,3]Thiadiazole-4-carbaldehyde (0.4 mmol). Acetic acid was added to bring the pH of the solution to 4, and the mixture was stirred for 15 minutes. Sodium triacetoxyborohydride (0.5 mmol) was then added and the reaction was stirred at RT overnight. The reaction mixture was stripped to dryness and the residue was purified via preparative HPLC, and lyophilized to give the product as a yellow solid (0.005 g, 7%). 1H NMR (400 MHz, MeOD) δ ppm 5.02 (s, 2 H) 7.25-7.28 (m, 1 H) 7.32 (t, J=8.72 Hz, 1 H) 7.44 (dd, J=6.57, 2.53 Hz, 1 H) 7.80 (d, J=2.53 Hz, 1 H) 8.38 (s, 1 H) 8.86 (s, 1 H).

### Example 303

### 8-Bromo-4-(3-chloro-4-fluoro-phenylamino)-6-({1-[2-(1,3-dioxo-1,3-dihydro-isoindol-2-yl)-ethyl]-1H-[1,2,3]triazol-4-ylmethyl}-amino)-quinoline-3-carbonitrile

In a 15 mL round-bottomed flask were added 6-amino-4-(4-bromo-3-fluoro-phenylamino)-quinoline-3-carbonitrile (0.18 g, 0.45 mmol), ethanol (15 mL) and 1-[2-(1,3-dioxo-1,3-dihydro-isoindol-2-yl)-ethyl]-1H-[1,2,3]triazole-4-carbaldehyde (1.2 mmol). Acetic acid was added to bring the pH of the solution to 4, and the mixture was stirred for 15 minutes. Sodium triacetoxyborohydride (1.2 mmol) was then added and the reaction was stirred at RT overnight. The reaction mixture was stripped to dryness and the residue was purified via preparative HPLC and lyophilized to give the product as a yellow solid (0.20 g, 70%). 1H NMR (400 MHz, DMSO-D6) δ ppm 3.92-4.04 (m, 2 H) 4.41 (d, J=5.56 Hz, 2 H) 4.56-4.68 (m, 2 H) 6.86 (t, J=6.32 Hz, 1 H) 7.27 (d, J=1.77 Hz, 2 H) 7.45 (t, J=8.97 Hz, 1 H) 7.52 (dd, J=6.57, 2.53 Hz, 1 H) 7.72-7.83 (m, 4 H) 8.09 (s, 1 H) 8.39 (s, 1 H) 9.46 (s, 1 H); HRMS (ESI+) calcd for C₂₉H₁₉BrClFN₈O₂ (MH+) 645.05596, found 645.0559.

### Example 304

### 6-{[1-(2-Amino-ethyl)-1H-[1,2,3]triazol-4-ylmethyl]-amino}-8-bromo-4-(3-chloro-4-fluoro-phenylamino)-quinoline-3-carbonitrile

8-Bromo-4-(3-chloro-4-fluoro-phenylamino)-6-({1-[2-(1,3-dioxo-1,3-dihydro-isoindol-2-yl)-ethyl]-1H-[1,2,3]triazol-4-ylmethyl}-amino)-quinoline-3-carbonitrile (0.20 g, 0.32 mmol) was added into a solution of hydrazine hydrate (1 mmol) in ethanol (10 mL). The mixture was heated to 60° C. for 4 h, the reaction mixture was stripped to dryness. The residue was purified via preparative HPLC and lyophilized to give the product as a yellow solid (0.105 g, 64%). 1H NMR (400 MHz, DMSO-D6) δ ppm 2.36 (s, 1 H) 2.57 (s, 1 H) 2.70 (s, 1 H) 3.12 (t, J=6.32 Hz, 2 H) 4.43-4.49 (m, 3 H) 7.26-7.32 (m, 2 H) 7.46 (t, J=8.97 Hz, 1 H) 7.52 (d, J=9.09 Hz, 1 H) 7.76 (d, J=2.27 Hz, 1 H) 8.04 (s, 1 H) 8.33 (s, 1 H) 8.39 (s, 1 H); HRMS (ESI+) calcd for C₂₁H₁₇BrClFN₈ (MH+) 515.05048, found 515.0511.

### Example 305

### 8-Bromo-4-(3-chloro-4-fluoro-phenylamino)-6-({1-[2-(1-methyl-pyrrolidin-2-yl)-ethyl]-1H-[1,2,3]triazol-4-ylmethyl}-amino)-quinoline-3-carbonitrile

In a 15 mL round-bottomed flask were added 6-amino-4-(4-bromo-3-fluoro-phenylamino)-quinoline-3-carbonitrile (0.04 g, 0.10 mmol), dichloroethane (2 mL) and 1-[2-(1-methyl-pyrrolidin-2-yl)-ethyl]-1H-[1,2,3]triazole-4-carbaldehyde (0.12mmol) The mixture was stirred for 15 minutes. Sodium triacetoxyborohydride (0.27 mmol) was then added and the reaction was stirred at RT for 5 h. The reaction mixture was stripped to dryness and the residue was purified via preparative HPLC and lyophilized to give the product as a yellow solid (0.04 g, 68%). 1H NMR (400 MHz, MeOD) δ ppm 2.08-2.42 (m, 5 H) 2.67-2.84 (m, 2 H) 3.31 (dd, J=45.98, 12.38 Hz, 2 H) 4.62-4.75 (m, 5 H) 7.34-7.48 (m, 3 H) 7.53-7.58 (m, 1 H) 7.84 (s, 1 H) 8.13 (s, 1 H) 8.44-8.52 (m, 3 H); HRMS (ESI+) calcd for C₂₆H₂₅BrClFN₈ (MH+) 583.11308, found 583.1147.

### Example 306

### 8-Bromo-4-(3-chloro-4-fluoro-phenylamino)-6-[(1-pyridin-3-ylmethyl-1 H-[1,2,3]triazol-4-ylmethyl)-amino]-quinoline-3-carbonitrile

In a 15 mL round-bottomed flask were added 6-amino-4-(4-bromo-3-fluoro-phenylamino)-quinoline-3-carbonitrile (0.04 g, 0.10 mmol), dichloroethane (2 mL) and 1-pyridin-3-ylmethyl-1H-[1,2,3]triazole-4-carbaldehyde (0.12 mmol). The mixture was stirred for 15 minutes. Sodium triacetoxyborohydride (0.27 mmol) was then added and the reaction was stirred at RT for 5 h. The reaction mixture was stripped to dryness and the residue was purified via preparative HPLC and lyophilized to give the product as a yellow solid (0.026 g, 46%). 1H NMR (400 MHz, MeOD) δ ppm 4.81 (s, 2 H) 5.94 (s, 2 H) 7.42-7.60 (m, 2 H) 7.68 (d, J=4.29 Hz, 1 H) 7.96 (s, 1 H) 8.05 (d, J=8.84 Hz, 1 H) 8.28 (s, 1 H) 8.59 (s, 1 H) 8.76-8.84 (m, 2 H); HRMS (ESI+) calcd for C₂₅H₁₇BrClFN₈ (MH+) 563.05048, found 563.0512.

### Example 307

### 6-{[1-(2-Azepan-1-yl-ethyl)-1H-[1,2,3]triazol-4-ylmethyl]-amino}-8-bromo-4-(3-chloro-4-fluoro-phenylamino)-quinoline-3-carbonitrile

In a 15 mL round-bottomed flask were added 6-amino-4-(4-bromo-3-fluoro-phenylamino)-quinoline-3-carbonitrile (0.04 g, 0.10 mmol), dichloroethane (2 mL) and 1-(2-azepan-1-yl-ethyl)-1H-[1,2,3]triazole-4-carbaldehyde (0.12 mmol). The mixture was stirred for 15 minutes. Sodium triacetoxyborohydride (0.27 mmol) was then added and the reaction was stirred at RT for 5 h. The reaction mixture was stripped to dryness and the residue was purified via preparative HPLC and lyophilized to give the product as a yellow solid (0.044 g, 73%). 1H NMR (400 MHz, MeOD) δ ppm 2.03 (d, J=54.32 Hz, 8 H) 3.45 (s, 4 H) 3.83 (d, J=3.79 Hz, 2 H) 4.92 (s, 2 H) 5.08 (d, J=5.81 Hz, 2 H) 7.54-7.68 (m, 3 H) 7.76 (dd, J=6.44, 2.15 Hz, 1 H) 8.05 (s, 1 H) 8.35 (s, 1 H) 8.65 (s, 1 H); HRMS (ESI+) calcd for C₂₇H₂₇BrClFN₈ (MH+) 597.12873, found 597.1304.

### Example 308

### 8-Bromo-4-(3-chloro-4-fluoro-phenylamino)-6-{[1-(2-pyrrolidin-1-yl-ethyl)-1H-[1,2,3]triazol-4-ylmethyl]-amino}-quinoline-3-carbonitrile

In a 15 mL round-bottomed flask were added 6-amino-4-(4-bromo-3-fluoro-phenylamino)-quinoline-3-carbonitrile (0.04 g, 0.10 mmol), dichloroethane (2 mL) and 1-(2-pyrrolidin-1-yl-ethyl)-1H-[1,2,3]triazole-4-carbaldehyde (0.12 mmol). The mixture was stirred for 15 minutes. Sodium triacetoxyborohydride (0.27 mmol) was then added and the reaction was stirred at RT for 5 h. The reaction mixture was stripped to dryness and the residue was purified via preparative HPLC, and lyophilized to give the product as a yellow solid (0.036 g, 64%). 1H NMR (400 MHz, MeOD) δ ppm 2.21 (s, 4 H) 2.86 (s, 2 H) 3.48 (m, 4 H) 3.92 (d, J=4.55 Hz, 2 H) 4.75 (s, 2 H) 7.38-7.51 (m, 3 H) 7.57 (d, J=2.02 Hz, 1 H) 7.88 (s, 1 H) 8.22 (s, 1 H) 8.48 (s, 2 H); HRMS (ESI+) calcd for C₂₅H₂₃BrClFN₈ (MH+) 569.09743, found 569.0987.

### Example 309

### 6-amino-4-[(3-chloro-4-fluorophenyl)amino]-8-(4-hydroxy-3-oxobutyl)quinoline-3-carbonitrile

In a 20 mL microwave vial were added 6-amino-4-(4-bromo-3-fluoro-phenylamino)-quinoline-3-carbonitrile (500 mg, 1.3 mmol), DMF (15 mL), but-3-ene-1,2-diol (114 mg, 1.3 mmol), palladium acetate (30 mg, 0.13 mmol), P(o-tol)₃ (60 mg, 0.26 mmol) and triethylamine (530 mg, 0.52 mmol). The reaction mixture was heated under microwave radiation at 180° C. for 20 min. The reaction mixture was diluted with water. The aqueous reaction mixture was washed with ethyl acetate (3×). The pooled ethyl acetate extracts were dried over magnesium sulfate and concentrated in vacuo. The residue was purified via preparative HPLC, and lyophilized to give the product as a yellow solid (84 mg, 16.2%). 1H NMR (400 MHz, DMSO-D6) δ ppm 2.83 (t, J=7.58 Hz, 2 H) 3.21 (t, J=7.58 Hz, 2 H) 4.06 (d, J=5.81 Hz, 2 H) 5.12 (t, J=5.94 Hz, 1 H) 5.71 (s, 2 H) 7.01 (d, J=2.27 Hz, 1 H) 7.08-7.15 (m, 2 H) 7.29-7.41 (m, 2 H) 8.38 (s, 1 H) 9.29 (s, 1 H).

### Example 310

### 4-[(3-chloro-4-fluorophenyl)amino]-8-(4-hydroxy-3-oxybutyl)-6-[(1H-imidazol-5-ylmethyl)amino]quinoline-3-carbonitrile

6-amino-4-[(3-chloro-4-fluorophenyl)amino]-8-(4-hydroxy-3-oxybutyl)quinoline-3-carbonitrile (81 mg, 0.20 mmol) was treated with sodium borohydride (16 mg, 0.40 mmol) in methanol (1 mL). The heterogeneous mixture, which turned to homogeneous solution upon addition of the reducing agent was allowed to stir at RT for 2 h. The solvent was rotavaped off and water was added to the crude reaction mixture. Dilute HCl was added to bring the pH of the aqueous solution to 4. The solid precipitated out which was filtered to give 4-[(3-chloro-4-fluorophenyl)amino]-8-(4-hydroxy-3-oxybutyl)-6-[(1H-imidazol-5-ylmethyl)amino]quinoline-3-carbonitrile in quantitative yields. This was used directly in the next step.

In a 15 mL round-bottom flask were added 6-amino-4-(4-bromo-3-fluoro-phenylamino)-quinoline-3-carbonitrile (82 mg, 0.20 mmol), ethanol (2 mL) and 1H-imidazole-5-carbaldehyde (20 mg, 0.20 mmol). Acetic acid was added to bring the pH of the solution to 4, and the mixture was stirred for 15 minutes. Sodium triacetoxyborohydride (43 mg, 0.40 mmol) was then added and the reaction was stirred at RT overnight. The reaction mixture was stripped to dryness and the residue was purified via preparative HPLC and lyophilized to give the product as a yellow solid (43 mg, 44.7%). 1H NMR (400 MHz, DMSO-D6) δ ppm 1.58 (s, 1 H) 1.82 (d, J=7.07 Hz, 1 H) 3.00-3.10 (m, 1 H) 3.10-3.23 (m, 4 H) 3.26-3.38 (m, 2 H) 3.41-3.52 (m, 2 H) 4.26 (s, 2 H) 7.06 (s, 2 H) 7.19-7.31 (m, 2 H) 7.36-7.49 (m, 2 H) 7.62 (s, 1 H) 8.35 (s,1 H).

### Example 311

### 8-bromo-4-[(3-chloro-4-fluorophenyl)amino]-6-{[(4-phenyl-1H-1,2,3-triazol-5-yl)methyl]amino}quinoline-3-carbonitrile

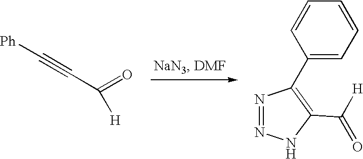

5-phenyl-1H-1,2,3-triazole-4-carbaldehyde was prepared by mixing 3-phenylpropiolaldehyde (250 mg, 1.92 mmol) and sodium azide (250 mg, 3.84 mmol) in 12 mL of DMF at RT for 4-5 h. In a 15 mL round-bottom flask were added 6-amino-4-(4-bromo-3-fluoro-phenylamino)-quinoline-3-carbonitrile (100 mg, 0.26 mmol), ethanol (1 mL) and 5-phenyl-1H-1,2,3-triazole-4-carbaldehyde (334 mg, 1.92 mmol). Acetic acid was added to bring the pH of the solution to 4, and the mixture was stirred for 15 minutes. Sodium triacetoxyborohydride (108 mg, 0.51 mmol) was then added and the reaction was stirred at RT overnight. The reaction mixture was stripped to dryness and the residue was purified via preparative HPLC and lyophilized to give the product as a yellow solid (12.7 mg, 8.9%). 1H NMR (400 MHz, DMSO-D6) δ ppm 4.54 (s, 2 H) 6.91 (d, J=4.04 Hz, 1 H) 7.25-7.57 (m, 8 H) 7.70-7.85 (m, 3 H) 8.42 (s, 1 H).

### Example 312

### Ethyl(2E)-3-{4-[(3-chloro-4-fluorophenyl)amino]-3-cyano-6-[(1H-imidazol-5-ylmethyl)amino]quinolin-8-yl}acrylate

In a 20 mL microwave vial were added 6-amino-4-(4-bromo-3-fluoro-phenylamino)-quinoline-3-carbonitrile (500 mg, 1.3 mmol), DMF (15 mL), ethyl acrylate (128 mg, 1.3 mmol), palladium acetate (30 mg, 0.13 mmol), P(o-tol)₃ (60 mg, 0.26 mmol) and triethylamine (530 mg, 0.52 mmol). The reaction mixture was heated under microwave radiation at 180° C. for 20 min. The reaction mixture was diluted with water. The aqueous reaction mixture was washed with ethyl acetate (3×). The pooled ethyl acetate extracts were dried over magnesium sulfate and concentrated in vacuo. The residue was purified via combiflash, and lyophilized to give the product as a yellow solid (162 mg, 30%).

In a 15 mL round-bottom flask were added E-ethyl-(6-amino-4-(3-chloro-4-fluorophenyl)amino)-3-cyanoquinolin-8-yl)acrylate (162 mg, 0.39 mmol), ethanol (2 mL) and 1H-imidazole-5-carbaldehyde (38 mg, 0.39 mmol). Acetic acid was added to bring the pH of the solution to 4, and the mixture was stirred for 15 minutes. Sodium triacetoxyborohydride (166 mg, 0.78 mmol) was then added and the reaction was stirred at RT overnight. The reaction mixture was stripped to dryness and the residue was purified via preparative HPLC and lyophilized to give the product as a yellow solid (1.8 mg, 0.94%). 1H NMR (400 MHz, DMSO-D6) δ ppm 1.29 (t, J=7.07 Hz, 3 H) 4.23 (q, J=7.07 Hz, 2 H) 4.31 (d, J=1.77 Hz, 2 H) 6.47 (d, 1 H) 6.63 (d, J=16.42 Hz, 1 H) 7.06 (d, J=4.55 Hz, 1 H) 7.26-7.31 (m, 1 H) 7.35-7.39 (m, 1 H) 7.45 (t, J=8.97 Hz, 1 H) 7.52 (dd, J=6.57, 2.78 Hz, 1 H) 7.63 (s, 1 H) 7.87 (s, 1 H) 8.42 (s, 1 H) 8.70 (d, J=16.42 Hz, 1 H) 9.48 (s,1 H).

### Example 313

### 1-[(3-chloro-4-fluorophenyl)amino]-5-[(1E)-3-hydroxyprop-1-en-1-yl]-7-[(1H-imidazol-5-ylmethyl)amino]-2-naphthonitrile

In a 15 mL round-bottom flask were added E-ethyl-(6-amino-4-(3-chloro-4-fluorophenyl)amino)-3-cyanoquinolin-8-yl)acrylate (65 mg, 0.16 mmol), THF (1 mL) and two equivalents of DIBAL-H (1 M solution in touluene). The reaction mixture was allowed to stir for 2 hours. The solvent was then evaporated to get the crude product, (E)-6-amino-4-(3-chloro-4-fluorophenylamino)-8-(3-hydroxyprop-1-enyl)quinoline-3-carbonitrile, which was used directly in the next step.

In a 15 mL round-bottom flask were added (E)-6-amino-4-(3-chloro-4-fluorophenylamino)-8-(3-hydroxyprop-1-enyl)quinoline-3-carbonitrile (0.16 mmol), ethanol (2 mL) and 1H-imidazole-5-carbaldehyde (38 mg, 0.39 mmol). Acetic acid was added to bring the pH of the solution to 4, and the mixture was stirred for 15 minutes. Sodium triacetoxyborohydride (166 mg, 0.78 mmol) was then added and the reaction was stirred at RT overnight. The reaction mixture was stripped to dryness and the residue was purified via preparative HPLC and lyophilized to give the product as a yellow solid (4.2 mg, 5.86%). 1H NMR (400 MHz, MeOD) δ ppm 2.12-2.17 (m, 2 H) 4.31 (dd, J=5.68, 1.64 Hz, 2 H) 4.44 (s, 1 H) 6.41-6.49 (m, 1 H) 7.07 (d, J=2.27 Hz, 1 H) 7.13-7.20 (m, 2 H) 7.24 (t, J=8.97 Hz, 1 H) 7.33 (dd, J=6.44, 2.65 Hz, 1 H) 7.51 (d, J=2.27 Hz, 1 H) 7.61 (d, J=15.92 Hz, 1 H) 8.07 (s, 1 H) 8.21 (s, 2 H) 8.30-8.35 (m, 1 H).

### Example 314

### 4-[(3-chloro-4-fluorophenyl)amino]-8-(2,3-dihydroxypropyl)-6-[(1H-imidazol-5-ylmethyl)amino]quinoline-3-carbonitrile

In a 20 mL microwave vial were added 6-amino-4-(4-bromo-3-fluoro-phenylamino)-quinoline-3-carbonitrile (500 mg, 1.3 mmol), DMF (15 mL), allyltributylstannane (630 mg, 1.9 mmol) and PdCl₂(PPh₃)₂ (100 mg, 0.13 mmol). The reaction mixture was heated under microwave radiation at 180° C. for 30 min. The reaction mixture was diluted with water. The aqueous reaction mixture was washed with ethyl acetate (3×). The pooled ethyl acetate extracts were dried over magnesium sulfate and concentrated in vacuo. The residue was purified via preparative HPLC, and lyophilized to give the product as a yellow solid (214 mg, 48%).

In a 15 mL round-bottom flask were added 8-allyl-6-amino-4-(3-chloro-4-fluorophenylamino)quinoline-3-carbonitrile (214 mg, 0.6 mmol), dichloromethane (3 mL), pyridine (119 mg, 1.5 mmol) and trifluoroacetic anhydride (265 mg, 1.3 mmol). The reaction was stirred at RT for 3-4 h. The reaction mixture was diluted with water and extracted with ethyl acetate (3×). The pooled ethyl acetate extracts were dried over magnesium sulfate and concentrated in vacuo. The crude product (258 mg, 0.48 mmol) was dissolved in 1:1 mixture of acetone and water (2 mL). To this were added osmiumtetraoxide (2.5 wt % in t-BuOH, 293 mg, 0.03 mmol) and NMO (112 mg, 0.96 mmol). The reaction mixture was stirred at RT for 4 h. To this was added 1 mL of 5M LiOH solution and stirred for another 3 h. The heterogeneous mixture was filtered through a pad of celite and washed with acetone and water. The filtrate was diluted with ethyl acetate and the two layers were separated. The aqueous reaction mixture was washed with ethyl acetate (2×). The pooled ethyl acetate extracts were dried over magnesium sulfate and concentrated in vacuo to give crude 6-amino-4-(3-chloro-4-fluorophenylamino)-8-(2,3-dihydroxypropyl)quinoline-3-carbonitrile, which was used directly for the next step.

In a 15 mL round-bottom flask were added 6-amino-4-(3-chloro-4-fluorophenylamino)-8-(2,3-dihydroxypropyl)quinoline-3-carbonitrile (186 mg, 0.48 mmol), ethanol (2 mL) and 1H-imidazole-5-carbaldehyde (51 mg, 0.53 mmol). Acetic acid was added to bring the pH of the solution to 4, and the mixture was stirred for 15 minutes. Sodium triacetoxyborohydride (204 mg, 0.96 mmol) was then added and the reaction was stirred at RT overnight. The reaction mixture was stripped to dryness and the residue was purified via preparative HPLC and lyophilized to give the product as a yellow solid (30.2 mg, 13.5%). 1H NMR (400 MHz, MeOD) δ ppm 2.02-2.04 (m, 1 H) 2.66 (s, 1 H) 3.14 (dd, J13.77, 7.71 Hz, 1 H) 3.35 (s, 1 H) 3.52 (d, J=5.31 Hz, 2 H) 3.94-4.02 (m, 1 H) 4.58 (s, 2 H) 7.13 (s, 1 H) 7.21-7.35 (m, 3 H) 7.38-7.47 (m, 2 H) 8.41 (s, 1 H) 8.81 (s, 1 H).

### Example 315

### 8-bromo-4-[(3-chloro-4-fluorophenyl)amino]-6-[(2H-1,2,3-triazol-4-ylmethyl)amino]quinoline-3-carbonitrile

In a microwave vial were added 3,3-diethoxy-prop-1-yne (1000 mg, 7.5 mmol), DEE (15 mL) and tributyltin azide (3240 mg, 9.76 mmol). The reaction mixture was heated under microwave radiation at 180° C. for 3 h. The solvent was evaporated under vacuo and the residue was treated with 2M HCl in MeOH for 16 h. The solvent was stripped down and the residue was dissolved in methanol and dichloroethane (20 mL). To this was added 6-amino-4-(4-bromo-3-fluoro-phenylamino)-quinoline-3-carbonitrile (500 mg, 1.3 mmol). Acetic acid was added to bring the pH of the solution to 4, and the mixture was stirred for 15 minutes. Sodium triacetoxyborohydride (542 mg, 2.55 mmol) was then added and the reaction was stirred at RT overnight. The reaction mixture was stripped to dryness and the residue was purified via preparative HPLC and lyophilized to give the product as a yellow solid (151.4 mg, 24%). 1H NMR (400 MHz, MeOD) δ ppm 4.54 (s, 2 H) 7.18 (d, J=2.53 Hz, 1 H) 7.21-7.31 (m, 2 H) 7.41 (dd, J=6.44, 2.40 Hz, 1 H) 7.69 (d, J=2.27 Hz, 2 H) 8.30 (s, 1 H).

### Example 316

### 8-bromo-4-[(3-chloro-4-fluorophenyl)amino]-6-{[(1-piperidin-4-yl-1H-1,2,3-triazol-4-yl)methyl]amino}quinoline-3-carbonitrile

In a 15 mL round-bottom flask were added 6-amino-4-(4-bromo-3-fluoro-phenylamino)-quinoline-3-carbonitrile (450 mg, 0.1.15 mmol), dichloroethane (4 mL) and tert-butyl 4-(4-formyl-1H-1,2,3-triazol-1-yl)piperidine-1-carboxylate (323 mg, 1.15 mmol). Acetic acid was added to bring the pH of the solution to 4, and the mixture was stirred for 15 minutes. Sodium triacetoxyborohydride (490 mg, 2.3 mmol) was then added and the reaction was stirred at RT overnight. The reaction mixture was stripped to dryness and the residue was purified via preparative HPLC and lyophilized to give the product, tert-butyl 4-(4-((8-bromo-4-(3-chloro-4-fluorophenylamino)-3-cyanoquinoline-6-ylamino)methyl)-1H-1,2,3-triazol-1-yl)piperidine-1-carboxylate as a yellow solid (516 mg, 64%).

tert-butyl 4-(4-((8-bromo-4-(3-chloro-4-fluorophenylamino)-3-cyanoquinoline-6-ylamino)methyl)-1H-1,2,3-triazol-1-yl)piperidine-1-carboxylate (511 mg, 0.73 mmol) was dissolved in 50% TFA solution in DCM. The reaction was allowed to stir for 2 h. The solvent was stripped down and the residue was diluted with ethyl acetate and sodium bicarbonate. The ethyl acetate layer was separated, dried over magnesium sulfate and concentrated in vacuo to give final product as a free base (318 mg, 78.5%). 1H NMR (400 MHz, MeOD) δ ppm 2.27-2.47 (m, 4 H) 3.19-3.29 (m, 2 H) 3.56 (d, J=13.39 Hz, 2 H) 4.56 (s, 2 H) 4.86 (d, J=4.04 Hz, 1 H) 7.32-7.46 (m, 3 H) 7.59 (dd, J=6.57, 2.27 Hz, 1 H) 7.75 (d, J=2.27 Hz, 1 H) 8.07 (s, 1 H) 8.43 (s, 1 H).

### Example 317

### 8-bromo-4-[(3-chloro-4-fluorophenyl)amino]-6-({[1-(1-methylpiperidin-4-yl)-1H-1,2,3-triazol-4-yl]methyl}amino)quinoline-3-carbonitrile

In a 15 mL round-bottom flask were added 8-bromo-4-[(3-chloro-4-fluorophenyl)amino]-6-{[(1-piperidin-4-yl-1H-1,2,3-triazol-4-yl)methyl]amino}quinoline-3-carbonitrile (90 mg, 0.16 mmol), dichloroethane (1 mL) and formaldehyde (14 mg, 0.17 mmol, 13 uL). Acetic acid was added to bring the pH of the solution to 4, and the mixture was stirred for 15 minutes. Sodium triacetoxyborohydride (68 mg, 0.32 mmol) was then added and the reaction was stirred at RT overnight. The reaction mixture was stripped to dryness and the residue was purified via preparative HPLC and lyophilized to give the product as a yellow solid (59.4 mg, 60.3%). 1H NMR (400 MHz, MeOD) δ ppm 2.26-2.37 (m, 4 H) 2.71 (s, 3 H) 2.86-2.96 (m, 2 H) 3.35-3.44 (m, 2 H) 4.52 (s, 2 H) 4.71 (s, 1 H) 7.16-7.35 (m, 3 H) 7.41 (dd, J=6.32, 2.27 Hz, 1 H) 7.67 (s, 1 H) 8.01 (s, 1 H) 8.27-8.46 (m, 2 H).

### Example 318

### 8-bromo-4-(3-chloro-4-fluorophenylamino)-6-((5-(2-hydroxypropyl)-3H-1,2,3-triazol-4-yl)methylamino)quinoline-3-carbonitrile

3,3-diethoxy-prop-1-yne (1000 mg, 7.6 mmol) was dissolved in ether (20 mL) in a 50 mL round bottom flask. n-BuLi (2.5M in hexanes; 7.6 mmol) was slowly added to the above solution. The reaction mixture was stirred and heated to reflux for 1 h. 2-methoxyoxirane (880 mg, 15.2 mmol) was then added to the above mixture. The stirring was continued at reflux temperatures for another 16 h. The reaction was cooled to RT and ether layer washed with water until neutral (3×). The ether layer was dried over magnesium sulfate and concentrated in vacuo. The crude product was then subjected to 2M HCl solution for 3 h. The solvent was removed in vacuo. The crude product was then dissolved in DMSO (10 mL) and treated with excess sodium azide. The reaction mixture was diluted with water. The aqueous reaction mixture was washed with ethyl acetate (3×). The pooled ethyl acetate extracts were dried over magnesium sulfate and concentrated in vacuo to give crude 5-(2-hydroxypropyl)-3H-1,2,3-triazole-4-carbaldehyde. The crude product was then taken up in ethanol (5 mL) and 6-amino-4-(4-bromo-3-fluoro-phenylamino)-quinoline-3-carbonitrile (200 mg, 0.5 mmol) was added. Acetic acid was added to bring the pH of the solution to 4, and the mixture was stirred for 15 minutes. Sodium triacetoxyborohydride (212 mg, 1 mmol) was then added and the reaction was stirred at RT overnight. The reaction mixture was stripped to dryness and the residue was purified via preparative HPLC and lyophilized to give the product as a yellow solid (14.3 mg, 5.3%). 1H NMR (400 MHz, MeOD) δ ppm 1.18 (d, J=6.06 Hz, 3 H) 2.75-2.95 (m, 2 H) 3.96-4.10 (m, 1 H) 4.49 (s, 2 H) 7.18 (s, 1 H) 7.21-7.31 (m, 2 H) 7.43 (dd, J=6.32, 1.77 Hz, 1 H) 7.65 (d, J-2.02 Hz, 1 H) 8.17 (s, 1 H) 8.27 (s, 1 H).

### Example 319

### 8-bromo-4-[(3-chloro-4-fluorophenyl)amino]-6-({[1-(2-hydroxy-3-morpholin-4-ylpropyl)-1H-1,2,3-triazol-4-yl]methyl}amino)quinoline-3-carbonitrile

In a 50 mL round-bottom flask were added 4-(oxiran-2-ylmethyl)morpholine (500 mg, 3.5 mmol), cerium(III) chloride (432 mg, 1.75 mmol), acetonitrile-water (9:1; 30 mL) and sodium azide (250 mg, 3.85 mmol). The mixture was heated to reflux for 5 h. The reaction mixture was diluted with water and extracted with ethyl acetate (3×). The pooled ethyl acetate extracts were dried over magnesium sulfate and concentrated in vacuo. The crude product was used directly in the next step.

The crude product of the above reaction was dissolved in water (10 mL) and 3,3-diethoxy-prop-1-yne (463 mg, 3.5 mmol), copper sulfate pentahydrate (5 mg, 0.09 mmol)) and ascorbic acid (18 mg, 0.18 mmol) were added. The heterogeneous mixture was allowed to stir overnight at RT. The mixture was cooled to 0° C. and conc. HCl was (1 mL) was added. After 5 h, the solvent was removed in vacuo.

The crude product of the above reaction was dissolved in dichloroethane (5 mL) and 6-amino-4-(4-bromo-3-fluoro-phenylamino)-quinoline-3-carbonitrile (100 mg, 0.26 mmol) was added. Acetic acid was added to bring the pH of the solution to 4, and the mixture was stirred for 15 minutes. Sodium triacetoxyborohydride (108 mg, 0.51 mmol) was then added and the reaction was stirred at RT overnight. The reaction mixture was stripped to dryness and the residue was purified via preparative HPLC and lyophilized to give the product as a yellow solid (2.3 mg, 1.5%). 1H NMR (400 MHz, MeOD) δ ppm 1.51-1.72 (m, 6 H) 2.38-2.51 (m, 2 H) 2.53-2.62 (m, 2 H) 3.47-3.58 (m, 2 H) 4.19 (s, 1 H) 4.38 (s, 1 H) 4.53 (s, 2 H) 7.42 (s, 1 H) 7.64-7.82 (m, 3 H) 7.95 (s, 1 H) 8.29 (s, 1 H).

### Example 320

### 2-(4-((8-chloro-4-(3-chloro-4-fluorophenylamino)-3-cyanoquinolin-6-ylamino)methyl)-1H-1,2,3-triazol-1-yl)-N-(pyridin-2-ylmethyl)acetamide

To a dry 15 mL round-bottomed flask was added with 2-(4-((8-chloro-4-(3-chloro-4-fluorophenylamino)-3-cyanoquinolin-6-ylamino)methyl)-1H-1,2,3-triazol-1-yl)acetic acid (73 mg, 0.15 mmol), BOP (100 mg, 0.225 mmol), DMF (2 mL). After stirring for 15 min, the mixture was added to pyridin-2-ylmethanamine (6 mmol) and stirred at room temperaturet for 4 h. The mixture was purified by HPLC to give 22 mg product. 1H NMR (400 MHz, DMSO-d₆) δ ppm 4.41 (d, 2 H) 4.48 (s, 2 H) 5.19 (s, 2 H) 7.25-7.34 (m, 4 H) 7.45 (t, J=8.84 Hz, 1 H) 7.54 (dd, J=6.44, 2.15 Hz, 1 H) 7.57 (s, 1 H) 7.76 (t, J=8.08 Hz, 1 H) 8.03 (s, 1 H) 8.38 (s, 1 H) 8.50 (d, J=6.32 Hz, 1 H)

### Example 321

### 8-chloro-4-(3-chloro-4-fluorophenylamino)-6-(methyl((1-(2-(1-methylpyrrolidin-2-yl)ethyl)-1H-1,2,3-triazol-4-yl)methyl)amino)quinoline-3-carbonitrile

In a test tube were added 8-chloro-4-(3-chloro-4-fluorophenylamino)-6-((1-(2-(1-methylpyrrolidin-2-yl)ethyl)-1H-1,2,3-triazol-4-yl)methylamino)quinoline-3-carbonitrile (0.020 g), dichloroethane (1 mL) and paraformaldehyde (excess). The mixture was stirred for 15 minutes. Sodium triacetoxyborohydride (excess) was then added and the reaction was stirred at room temperature overnight. The reaction mixture was stripped to dryness and the residue was purified via preparative HPLC, and lyophilized to give 0.011 g product. 1H NMR (400 MHz, MeOD) δ ppm 1.66-1.85 (m, 1 H) 1.99-2.41 (m, 3 H) 2.61-2.76 (m, 1 H) 2.96 (s, 3 H) 3.12-3.24 (m, 1 H) 3.64-3.77 (m, 1 H) 4.67 (s, 2 H) 7.34-7.62 (m, 3 H) 8.06 (s, 1 H) 8.12 (s, 1 H) 8.48 (s, 1 H) 8.59 (s, 1 H)

### Example 322

### tert-butyl 4-((8-chloro-4-(3-chloro-4-fluorophenylamino)-3-cyanoquinolin-6-ylamino)methyl)piperidine-1-carboxylate

In a 25 mL round-bottomed flask were added 6-amino-8-chloro-4-(3-chloro-4-fluoro-phenylamino)-quinoline-3-carbonitrile (0.35 g, 1 mmol), dichloroethane (5 mL) and tert-butyl 4-formylpiperidine-1-carboxylate (2.5 mmol). The mixture was stirred for 15 minutes. Sodium triacetoxyborohydride (3 mmol) was then added and the reaction was stirred at rt for 24 h. The reaction mixture was stripped to dryness and the residue was purified via preparative HPLC, and lyophilized to give 0.5 g product as a yellow solid. 1H NMR (400 MHz, DMSO-d₆) δ ppm 0.97-1.13 (m, 2 H) 1.39 (s, 9 H) 1.64-1.81 (m, 2 H) 2.63-2.78 (m, 1 H) 3.03 (d, J=6.57 Hz, 2 H) 4.07-4.10 (m, 4 H) 6.98 (d, J=2.02 Hz, 1 H) 7.27 (d, J=3.03 Hz, 1 H) 7.41-7.49 (m, 2 H) 7.51 (d, J=2.27 Hz, 1 H) 8.35 (s, 1 H)

### Example 323

### 8-chloro-4-(3-chloro-4-fluorophenylamino)-6-((1-(pyridin-3-ylmethyl)piperidin-4-yl)methylamino)quinoline-3-carbonitrile

To a 15 mL round-bottomed flask was added tert-butyl 4-((8-chloro-4-(3-chloro-4-fluorophenylamino)-3-cyanoquinolin-6-ylamino)methyl)piperidine-1-carboxylate(0.05 g), methylene chloride (2 mL) and TFA (0.5 mL). The reaction was stirred at room temperature for 2 h, and the solvent was removed in vacuo. The residue was dissolved in dichloroethane and nicotinaldehyde (1.5 equiv.) was added. After 15 min, sodium borotriacetoxyhydride (excess) was added and stirred at room temperature for 5 h. The reaction mixture was stripped to dryness and the residue was purified via preparative HPLC, and lyophilized to give 0.033 g product as a yellow solid. 1H NMR (400 MHz, MeOD) δ ppm 1.74-1.93 (m, 2 H) 2.18-2.30 (m, 1 H) 2.32-2.42 (m, 2 H) 3.17 (s, 2 H) 3.48 (d, J=6.57 Hz, 2 H) 3.75 (s, 2 H) 4.54 (s, 2 H) 7.32 (d, J=2.02 Hz, 1 H) 7.52-7.59 (m, 1 H) 7.63 (t, J=8.84 Hz, 1 H) 7.70-7.78 (m, 2 H) 7.87 (dd, J=7.96, 4.93 Hz, 1 H) 8.31 (dd, J=7.96, 1.64 Hz, 1 H) 8.64 (s, 1 H) 8.91-9.06 (m, 2 H)

### Example 324

### (S)-8-bromo-4-(3-chloro-4-fluorophenylamino)-6-((1-(2-(1-methylpyrrolidin-2-yl)ethyl)-1H-1,2,3-triazol-4-yl)methylamino)quinoline-3-carbonitrile

In a 15 mL round-bottomed flask were added 6-amino-8-bromo-4-(3-chloro-4-fluoro-phenylamino)-quinoline-3-carbonitrile (0.34 g, 1 mmol), dichloroethane (6 mL) and 1-[2-(1-Methyl-pyrrolidin-2-yl)-ethyl]-1H-[1,2,3]triazole-4-carbaldehyde (1.2 mmol). The mixture was stirred for 15 minutes. Sodium triacetoxyborohydride (2.4 mmol) was then added and the reaction was stirred at room temperature for 24 h. The reaction mixture was stripped to dryness and the residue was purified via preparative HPLC, and lyophilized to give 8-Bromo-4-(3-chloro-4-fluoro-phenylamino)-6-({1-[2-(1-methyl-pyrrolidin-2-yl)-ethyl]-1H-[1,2,3]triazol-4-ylmethyl}-amino)-quinoline-3-carbonitrile as a yellow solid (0.3 g). The product was then separated by chiral HPLC to give (R)-8-Bromo-4-(3-chloro-4-fluoro-phenylamino)-6-({1-[2-(1-methyl-pyrrolidin-2-yl)-ethyl]-1H-[1,2,3]triazol-4-ylmethyl}-amino)-quinoline-3-carbonitrile (75 mg) and (S)-8-bromo-4-(3-chloro-4-fluorophenylamino)-6-((1-(2-(1-methylpyrrolidin-2-yl)ethyl)-1H-1,2,3-triazol-4-yl)methylamino)quinoline-3-carbonitrile (88 mg). 1H NMR (400 MHz, MeOD) δ ppm 1.48-1.68 (m, 2 H) 1.84-2.21 (m, 4 H) 2.25-2.60 (m, 6 H) 3.21-3.29 (m, 1 H) 4.58-4.73 (m, 2 H) 4.75 (s, 2 H) 7.35-7.40 (m, 1 H) 7.42-7.47 (m, 1 H) 7.50 (t, J=8.72 Hz, 1 H) 7.59-7.67 (m, 1 H) 7.89 (d, J=2.53 Hz, 1 H) 8.15 (s, 1 H) 8.51 (s,1 H); 1H NMR (400 MHz, MeOD) δ ppm 1.47-1.56 (m, 5 H) 1.65-1.81 (m, 1 H) 1.96-2.11 (m, 3 H) 2.22-2.36 (m, 1 H) 2.73 (s, 3 H) 3.19-3.33 (m, 3 H) 4.75 (s, 2 H) 7.39 (d, J=2.27 Hz, 1 H) 7.42-7.48 (m, 1 H) 7.50 (t, J=8.72 Hz, 1 H) 7.59-7.67 (m, 1 H) 7.89 (d, J=2.27 Hz, 1 H) 8.18 (s, 1 H) 8.51 (s, 1 H)

### Example 325

### 8-chloro-4-(3-chloro-4-fluorophenylamino)-6-((1-(1-cyclobutylpiperidin-4-yl)-1H-1,2,3-triazol-4-yl)methylamino)quinoline-3-carbonitrile

In a test tube were added 8-chloro-4-(3-chloro-4-fluorophenylamino)-6-((1-(piperidin-4-yl)-1H-1,2,3-triazol-4-yl)methylamino)quinoline-3-carbonitrile (50 mg), dichloroethane (2 mL) and cyclobutanone(1.2 equiv.). The mixture was stirred for 15 minutes. Sodium triacetoxyborohydride (2 equiv.) was then added and the reaction was stirred at room temperature overnight. The reaction mixture was stripped to dryness and the residue was purified via preparative HPLC, and lyophilized to give 8-chloro-4-(3-chloro-4-fluorophenylamino)-6-((1-(1-cyclobutylpiperidin-4-yl)-1H-1,2,3-triazol-4-yl)methylamino)quinoline-3-carbonitrile (0.047 g). 1H NMR (400 MHz, MeOD) δ ppm 2.00-2.14 (m, 2 H) 2.32-2.42 (m, 2 H) 2.46-2.61 (m, 5 H) 2.95-3.09 (m, 2 H) 3.59-3.75 (m, 3 H) 4.76 (s, 2 H) 4.90-5.01 (m, 1 H) 7.37 (d, J=2.27 Hz, 1 H) 7.43-7.49 (m, 1 H) 7.51 (t, J=8.72 Hz, 1 H) 7.64 (dd, J=6.44, 2.40 Hz, 1 H) 7.70 (d, J=2.53 Hz, 1 H) 8.21 (s, 1 H) 8.53 (s, 1 H)

### Example 326

### N-[2-({8-chloro-4-[(3-chloro-4-fluorophenyl)amino]-3-cyanoquinolin-6-yl}amino)ethyl]methanesulfonamide

Step 1. In a microwave tube, 2,2-dimethoxyethylamine (0.15 g, 1.44 mmol) was taken up in 5 mL DCM, and Hunig's base (0.50 mL, 2.88 mmol) was added. Methane sulfonylchloride (165.0 mg, 1.44 mmol) was then added, and the reaction mixture was stirred at room temperature for 3 hours. The reaction was monitored by TLC. The pH was adjusted to 1-2 by adding HCl (1.25 M in MeOH), then 7-8 drops of H₂O was added. The reaction mixture was heated to 80° C. for 10 min in a microwave. Solvent was reduced to minimum volume. This mixture containing crude aldehyde N-(2-Oxo-ethyl)-methanesulfonamide was used for next step synthesis without purification.

Step 2. The procedure described above for the synthesis of 6-((1H-imidazol-4-yl)methylamino)-4-(3-chloro-4-fluorophenylamino)quinoline-3-carbonitrile was followed, reacting 6-amino-8-chloro-4-(3-chloro-4-fluorophenylamino)quinoline-3-carbonitrile (100 mg, 0.29 mmol) with the crude aldehyde, HOAc (200 uL), and NaCNBH₃ (12.7 mg, 0.20 mmol) in 3 mL EtOH. Purification using preparative-HPLC gave a yellow solid as product (2.5 mg, 1.9% yield). 1H NMR (400 MHz, MeOD) δ ppm 1.94 (s, 2 H) 2.85 (s, 3 H) 3.25-3.37 (m, 2 H) 6.99-7.04 (m, J=2.27 Hz, 1 H) 7.14-7.23 (m, 2 H) 7.30-7.39 (m, 2 H) 8.21 (s, 1 H); HRMS (ESI+) calcd for C₁₉H₁₆Cl₂FN₅O₂S (MH+) 468.04585, found 468.0462.

### Example 327

### 8-chloro-4-[(3-chloro-4-fluorophenyl)amino]-6-({[1-(1-ethylpiperidin-4-yl)-1H-1,2,3-triazol-4-yl]methyl}amino)quinoline-3-carbonitrile

In a 15 mL round-bottom flask were added 8-chloro-4-[(3-chloro-4-fluorophenyl)amino]-6-{[(1-piperidin-4-yl-1H-1,2,3-triazol-4-yl)methyl]amino}quinoline-3-carbonitrile (75 mg, 0.15 mmol), dichloroethane (1 mL) and acetaldehyde (7 mg, 0.15 mmol). Acetic acid was added to bring the pH of the solution to 4, and the mixture was stirred for 15 minutes. Sodium triacetoxyborohydride (64 mg, 0.3 mmol) was then added and the reaction was stirred at room temperature overnight. The reaction mixture was stripped to dryness and the residue was purified via preparative HPLC, and lyophilized to give the product as a yellow solid (9.4 mg, 10.7%). 1H NMR (400 MHz, CHLOROFORM-D) δ ppm 1.31 (t, J=7.20 Hz, 3 H) 2.29-2.41 (m, 3 H) 2.78-2.92 (m, 2 H) 2.93-3.03 (m, 2 H) 3.39-3.51 (m, 2 H) 4.54 (s, 2 H) 7.08-7.16 (m, 1 H) 7.20-7.29 (m, 2 H) 7.34-7.41 (m, 1 H) 7.43-7.49 (m, 1 H) 7.65-7.68 (m, 1 H) 8.29-8.38 (m, 2 H). HRMS: calcd for C₂₆H₂₅Cl₂FN₈+H+, 539.16360; found (ESI-FTMS, [M+H]¹⁺), 539.162

### Example 328

### 8-chloro-4-[(3-chloro-4-fluorophenyl)amino]-6-({[1-(1-propylpiperidin-4-yl)-1H-1,2,3-triazol-4-yl]methyl}amino)quinoline-3-carbonitrile

In a 15 mL round-bottom flask were added 8-chloro-4-[(3-chloro-4-fluorophenyl)amino]-6-{[(1-piperidin-4-yl-1H-1,2,3-triazol-4-yl)methyl]amino}quinoline-3-carbonitrile (100 mg, 0.2 mmol), dichloroethane (1 mL) and propanal (12 mg, 0.2 mmol). Acetic acid was added to bring the pH of the solution to 4, and the mixture was stirred for 15 minutes. Sodium triacetoxyborohydride (85 mg, 0.4 mmol) was then added and the reaction was stirred at room temperature overnight. The reaction mixture was stripped to dryness and the residue was purified via preparative HPLC, and lyophilized to give the product as a yellow solid (68.8 mg, 57.5%). 1H NMR (400 MHz, MeOD) δ ppm 1.02 (t, J=7.33 Hz, 3 H) 1.69-1.84 (m, 2 H) 2.33-2.51 (m, 4 H) 2.98-3.08 (m, 2 H) 3.09-3.20 (m, 2 H) 3.61 (d, J=12.38 Hz, 2 H) 4.52 (s, 2 H) 4.75-4.87 (m, 1 H) 7.08-7.16 (m, 1 H) 7.19-7.30 (m, 2 H) 7.35-7.47 (m, 2 H) 8.03 (s, 1 H) 8.26 (s, 1 H) 8.34 (s, 4 H). HRMS: calcd for C₂₇H₂₇Cl₂FN₈+H+, 553.17925; found (ESI-FTMS, [M+H]¹⁺), 553.1817

### Example 329

### 8-chloro-4-[(3-chloro-4-fluorophenyl)amino]-6-({[1-(1-methylazepan-4-yl)-1H-1,2,3-triazol-4-yl]methyl}amino)quinoline-3-carbonitrile

In a 15 mL round-bottom flask were added 6-{[(1-azepan-4-yl-1H-1,2,3-triazol-4-yl)methyl]amino}-8-chloro-4-[(3-chloro-4-fluorophenyl)amino]quinoline-3-carbonitrile (90 mg, 0.17 mmol), dichloroethane (1 mL) and formaldehyde (14 mg, 0.17 mmol). Acetic acid was added to bring the pH of the solution to 4, and the mixture was stirred for 15 minutes. Sodium triacetoxyborohydride (72 mg, 0.34 mmol) was then added and the reaction was stirred at room temperature overnight. The reaction mixture was stripped to dryness and the residue was purified via preparative HPLC, and lyophilized to give the product as a yellow solid (23.5 mg, 23.6%). 1H NMR (400 MHz, MeOD) δ ppm 1.91-2.05 (m, 1 H) 2.05-2.27 (m, 2 H) 2.28-2.61 (m, 3 H) 2.89 (s, 3 H) 3.33-3.45 (m, 3 H) 3.47-3.59 (m, 1 H) 4.51 (s, 2 H) 7.07-7.16 (m, 1 H) 7.19-7.30 (m, 2 H) 7.34-7.45 (m, 2 H) 7.99 (s, 1 H) 8.24 (s,1 H) 8.47 (s, 1 H). HRMS: calcd for C₂₆H₂₅Cl₂FN₈+H+, 539.16360; found (ESI-FTMS, [M+H]¹⁺), 539.166

### Example 330

### 8-chloro-4-[(3-chloro-4-fluorophenyl)amino]-6-({[1-(1-ethylazepan-4-yl)-1H-1,2,3-triazol-4-yl]methyl}amino)quinoline-3-carbonitrile

In a 15 mL round-bottom flask were added 6-{[(1-azepan-4-yl-1H-1,2,3-triazol-4-yl)methyl]amino}-8-chloro-4-[(3-chloro-4-fluorophenyl)amino]quinoline-3-carbonitrile (90 mg, 0.17 mmol), dichloroethane (1 mL) and acetaldehyde (8 mg, 0.17 mmol). Acetic acid was added to bring the pH of the solution to 4, and the mixture was stirred for 15 minutes. Sodium triacetoxyborohydride (72 mg, 0.34 mmol) was then added and the reaction was stirred at room temperature overnight. The reaction mixture was stripped to dryness and the residue was purified via preparative HPLC, and lyophilized to give the product as a yellow solid (25.4 mg, 25%). 1H NMR (400 MHz, MeOD) δ ppm 1.27-1.39 (m, 4 H) 1.89-2.04 (m, 1 H) 2.05-2.25 (m, 2 H) 2.27-2.62 (m, 3 H) 3.15-3.26 (m, 2 H) 3.32-3.46 (m, 3 H) 3.47-3.57 (m, 1 H) 4.52 (s, 2 H) 7.10-7.16 (m, 1 H) 7.19-7.30 (m, 2 H) 7.36-7.47 (m, 2 H) 7.98 (s, 1 H) 8.28 (s, 1 H) 8.49 (s, 1 H). HRMS: calcd for C₂₇H₂₇Cl₂FN₈+H+, 553.17925; found (ESI-FTMS, [M+H]¹⁺), 553.1816

### Example 331

### 8-chloro-4-[(3-chloro-4-fluorophenyl)amino]-6-({[1-(1-isopropylazepan-4-yl)-1H-1,2,3-triazol-4-yl]methyl}amino)quinoline-3-carbonitrile

In a 15 mL round-bottom flask were added 6-{[(1-azepan-4-yl-1H-1,2,3-triazol-4-yl)methyl]amino}-8-chloro-4-[(3-chloro-4-fluorophenyl)amino]quinoline-3-carbonitrile (90 mg, 0.17 mmol), dichloroethane (1 mL) and acetone (excess). Acetic acid was added to bring the pH of the solution to 4, and the mixture was stirred for 15 minutes. Sodium triacetoxyborohydride (72 mg, 0.34 mmol) was then added and the reaction was stirred at room temperature overnight. The reaction mixture was stripped to dryness and the residue was purified via preparative HPLC, and lyophilized to give the product as a yellow solid (67.2 mg, 64.4%). 1 H NMR (400 MHz, MeOD) δ ppm 1.35 (d, J=6.57 Hz, 6 H) 1.91-2.06 (m, 1 H) 2.08-2.39 (m, 3 H) 2.41-2.61 (m, 2 H) 3.33-3.42 (m, 3 H) 3.43-3.53 (m, 1 H) 3.54-3.67 (m, 1 H) 4.51 (s, 2 H) 7.08-7.16 (m, 1 H) 7.18-7.30 (m, 2 H) 7.34-7.45 (m, 2 H) 8.25 (s, 1 H) 8.52 (s, 1 H). HRMS: calcd for C₂₈H₂₉Cl₂FN₈+H+, 567.19490; found (ESI-FTMS, [M+H]¹⁺), 567.1962

### Example 332

### 8-chloro-4-[(3-chloro-4-fluorophenyl)amino]-6-({[1-(1,4-dioxaspiro[4.5]dec-8-yl)-1H-1,2,3-triazol-4-yl]methyl}amino)quinoline-3-carbonitrile

Step 1: In a round bottom flask was added 1,4-Dioxaspiro{4,5]decan-5-ol (5 g, 31.6 mmol), 20 ml Dichloromethane, and DIEA (7.1 ml, 37.9 mmol), then the mixture was cooled to 0° C. and mesyl chloride (2.9 ml, 34.8 mmol) was added dropwise. The reaction was then warmed to room temperature. After five hours of stirring, the reaction was diluted with dichloromethane and extracted three times with saturated sodium bicarbonate. The organic phase was dried with MgSO₄, filtered and evaporated under reduced pressure to give the product as an oil (7.53 g, 93.53%).

Step 2: To the crude from Step 1 was added sodium azide (5.13 g, 78.9 mmol), and 35 ml DMF and the mixture was stirred at 120° C. overnight. The reaction was extracted with chloroform/sat. NaHCO₃. The organic layer was dried with MgSO₄, filtered and the solvent was removed under reduced pressure (4.6 g, 79.3%).

Step 3: To the crude from Step 2 was added 25 ml DMF, 20 ml water, propiolaldehyde diethylacetal (5.4ml, 38 mmol), Na L-Ascorbate (250 mg, 1.3 mmol) and CuSO₄ (316 mg, 1.3 mmol). The mixture was stirred at 40° C. for two hours and then at room temperature overnight. The reaction mixture was extracted with chloroform/saturated NaHCO₃. The organic layer was dried with MgSO₄, filtered and the solvent was removed under reduced pressure.

Step 4: The procedure described above for the synthesis of 4-[(3-chloro-4-fluorophenyl)amino]-6-[(1H-imidazol-5-ylmethyl)amino]quinoline-3-carbonitrile was followed, reacting 6-amino-4-[(3-chloro-4-fluorophenyl)amino]-8-(chloro)quinoline-3-carbonitrile (2 g, 5.76 mmol) with the crude from Step 3 (254 mg, 13.5 mmol) and NaCNBH₃ (362.0 mg, 5.76 mmol) in 40 mL DMF. The reaction was filtered and purified via preparative HPLC, and lyophilized to give the product as a yellow solid (0.8 g, 36.7%): 1H NMR (400 MHz, DMSO-D6) δ ppm 1.64-1.79 (m, 4 H) 1.93-2.03 (m, 4 H) 3.89 (s, 4 H) 4.42 (d, J=5.31 Hz, 2 H) 4.53-4.64 (m, 1 H) 6.88 (t, J=5.31 Hz, 1 H) 7.24 (d, J=2.27 Hz, 1 H) 7.26-7.32 (m, 1 H) 7.46 (t, J=8.97 Hz, 1 H) 7.53 (dd, J=6.44, 2.65 Hz, 1 H) 7.56 (d, J=2.27 Hz, 1 H) 8.13 (s, 1 H) 8.40 (s, 1 H) 9.48 (s, 1 H); HRMS: calcd for C₂₇H₂₄Cl₂FN₇O₂+H+, 568.14253; found (ESI-FTMS, [M+H]¹⁺), 568.14101.

### Example 333

### 8-chloro-4-[(3-chloro-4-fluorophenyl)amino]-6-({[1-(4-oxocyclohexyl)-1H-1,2,3-triazol-4-yl]methyl}amino)quinoline-3-carbonitrile

In a round bottom flask was added 8-chloro-4-[(3-chloro-4-fluorophenyl)amino]-6-({[1-(1,4-dioxaspiro[4.5]dec-8-yl)-1H-1,2,3-triazol-4-yl]methyl}amino)quinoline-3-carbonitrile (400 mg, 0.7 mmol), 8 ml TFA, 1 ml Acetone and 1 ml water, and the mixture was stirred overnight. The reaction was filtered and purified via preparative HPLC, and lyophilized to give the product as a yellow solid (234 mg, 63.4%): 1H NMR (400 MHz, DMSO-D6) δ ppm 1.01-1.11 (m, 3 H) 1.21-1.36 (m, 1 H) 1.57-1.84 (m, 5 H) 1.97-2.08 (m, 2 H) 2.08-2.21 (m, 1 H) 2.54-2.64 (m, 1 H) 2.67-2.73 (m, 1 H) 4.37-4.55 (m, 3 H) 6.68 (s, 1 H) 6.90 (t, J=5.81 Hz, 1 H) 7.22-7.33 (m, 2 H) 7.45 (t, J=9.09 Hz, 1 H) 7.50-7.55 (m, 1 H) 7.56 (t, J=2.40 Hz, 1 H) 8.11 (d, J=19.20 Hz, 1 H) 8.40 (d, J=2.27 Hz, 1 H) 9.50 (s, 1 H): HRMS: calcd for C₂₅H₂₀Cl₂FN₇O+H+, 524.11632; found (ESI-FTMS, [M+H]¹⁺), 524.11386.

### Example 334

### 8-chloro-4-[(3-chloro-4-fluorophenyl)amino]-6-{[(1-phenyl-1H-1,2,3-triazol-4-yl)methyl]amino}quinoline-3-carbonitrile

Step 1: The procedure described above for the synthesis of 6-{[(1-tert-butyl-1H-1,2,3-triazol-4-yl)methyl]amino)-8-chloro-4-[(3-chloro-4-fluorophenyl)amino]quinoline-3-carbonitrile was followed, reacting (1-phenyl-1H-1,2,3-triazol-4-yl)methanol (576 mg, 3.31 mmol), 4 mL methylene chloride, 4 mL DME and 576 mg MnO₂. Purification by flash chromatography (1 to 1.5% MeOH/DCM) gave the product (400 mg, 70.2%).

Step 2: In a 50 ml round bottom flask was added 10 ml anhydrous MeOH, 1 ml DIEA, 630 mg 2,2,2-Trifluoroethylamine HCl and the product from Step1 and the mixture was stirred at room temperature overnight under nitrogen flow. MeOH was removed under reduced pressure. The salts were washed with water/EtOAc and then with brine. The EtOAc phase was dried with MgSO₄ and filtered. The solvent was removed under reduced pressure to give the product as a yellow solid (˜440 mg, ˜74.8%).

Step 3: In a Microwave tube was added crude from step 2 in ˜3 g of DMSO. Replacement reaction was done in a microwave reactor at 140° C. for 15 min. After extraction with brine/EtOAc, the organic layer was dried over MgSO₄ and filtered and then the solvent was removed under reduced pressure. The crude product was used in the next step.

Step 4: In a 50 ml round bottom flask was added the crude from step 3 in 15 ml MeOH, 4.0 ml formic acid (96%) and 11 ml water and refluxed overnight at 80° C. The reaction was then cooled to room temperature and 50 ml water was added. The product was extracted three times with chloroform, and the chloroform extracts were washed with water and dried over MgSO₄. The solvent was removed under reduced pressure and the crude was used in the next step.

Step 5: The procedure described above for the synthesis of 4-[(3-chloro-4-fluorophenyl)amino]-6-[(1H-imidazol-5-ylmethyl)amino]quinoline-3-carbonitrile was followed, reacting 6-amino-4-[(3-chloro-4-fluorophenyl)amino]-8-(chloro)quinoline-3-carbonitrile (50.0 mg, 0.14 mmol) with the crude from Step 4 and NaCNBH₃ (17.4 mg, 0.28 mmol) in 10 mL EtOH. The reaction was stripped to dryness and the residue was purified via preparative HPLC, and lyophilized to give the product as a yellow solid (28.1 mg, 38.7%): 1H NMR (400 MHz, DMSO-D6) δ ppm 4.55 (d, J=5.56 Hz, 2 H) 6.98 (t, J=5.18 Hz, 1 H) 7.25-7.33 (m, 2 H) 7.45 (t, J=8.97 Hz, 1 H) 7.48-7.52 (m, 1 H) 7.54 (dd, J=6.32, 2.27 Hz, 1 H) 7.56-7.62 (m, 3 H) 7.84-7.89 (m, 2 H) 8.40 (s, 1 H) 8.76 (s, 1 H) 9.51 (s, 1 H); HRMS (ESI+) calcd for C₂₅H₁₆Cl₂FN₇ (MH+) 504.09010, found 504.09.

### Example 335

### 8-chloro-4-[(3-chloro-4-fluorophenyl)amino]-6-({[1-(4-hydroxy-4-pyridin-2-ylcyclohexyl)-1H-1,2,3-triazol-4-yl]methyl}amino)quinoline-3-carbonitrile

Step 1: 25 μL 2-Bromopyridine was mixed into 5 ml THF, cooled to −78° C. and 105 μL solution of Butyl lithium (2.5M in Hexanes) was added dropwise. The reaction was stirred for 15 minutes, then a solution of 4-[4-(diethoxymethyl)-1H-1,2,3-triazol-1-yl]cyclohexanone (70.4 mg in 3 ml THF) was added over five minutes under nitrogen flow. The reaction was stirred 1 h at −78° C. and 1 h at room temperature. The solvent was removed under reduced pressure to yield brown oil.

Step 2: To the crude from Step 1 was added 6 ml of a 1.25M solution of hydrochloric acid in methanol and 2 ml water. The mixture was then refluxed for an hour and the solvents were removed under reduced pressure to yield brown oil.

Step 3: The procedure described above for the synthesis of 4-[(3-chloro-4-fluorophenyl)amino]-6-[(1H-imidazol-5-ylmethyl)amino]quinoline-3-carbonitrile was followed, reacting 6-amino-4-[(3-chloro-4-fluorophenyl)amino]-8-(chloro)quinoline-3-carbonitrile (49.5 mg, 0.142 mmol) with the crude from Step 2 and NaCNBH₃ (25 mg, 0.4 mmol) in 4 mL DMF. The reaction was filtered and purified via preparative HPLC, and lyophilized to give the product as a yellow solid (3.0 mg, 3.49%). 1H NMR (400 MHz, DMSO-d₆) δ ppm 1.52-1.62 (m, 1 H) 1.67-1.75 (m, 1 H) 1.92-2.01 (m, 2 H) 2.04-2.31 (m, 4 H) 4.45 (d, J=4.80 Hz, 2 H) 4.60-4.70 (m, 1 H) 5.26 (d, J=16.42 Hz, 1 H) 6.88 (s, 1 H) 7.18-7.36 (m, 3 H) 7.37-7.61 (m, 3 H) 7.61-7.73 (m, 1 H) 7.74-7.85 (m, 1 H) 8.14 (d, J=9.09 Hz, 1 H) 8.34-8.44 (m, 1 H) 8.45-8.55 (m, 1 H) 9.51 (s, 1 H); HRMS: calcd for C₃₀H₂₅Cl₂FN₈O+H+, 603.15852; found (ESI-FTMS, [M+H]¹⁺), 603.15706.

Biological Testing

To determine whether Tpl2 inhibitors may be efficacious in the treatment of rheumatoid arthritis, as well as other inflammatory disease states, an N-terminal 6His-tagged human Cot/Tpl2 kinase construct encoding residues 30-398 was expressed in a baculovirus system (BD Biosciences, San Jose, Calif.). Sf9 cells expressing the kinase were lysed in 50 mM NaPhosphate pH=8; 300 mM NaCl; 5 mM imidazole; 0.1 mM EGTA; 25 mM beta-glycerophosphate; 1% TX-100, 1% glycerol; 6 mM beta-mercaptoethanol and protease inhibitors. The lysate was clarified by centrifugation and was loaded onto a Ni-Sepharose column. The column was washed with 50 mM NaPhosphate pH=8; 300 mM NaCl; 15 mM imidazole; 1% glycerol; and 6 mM beta-mercaptoethanol. His-Tpl2 was eluted with 50 mM NaPhosphate pH=8; 300 mM NaCl; 250 mM imidazole; 1% glycerol; and 6 mM beta-mercaptoethanol. The eluted protein was further purified by size exclusion chromatography. Fractions corresponding to monomeric Tpl2 were then used in the assay.

Tpl2/Cot activity was directly assayed using GST-MEK1 as a substrate. GST-MEK1 phosphorylation on serine residues 217 and 221 was detected by ELISA. 0.4 nM Tpl2 was incubated with 35 nM GST-MEK1 in a kinase reaction buffer containing 20 mM MOPS pH=7.2; 50 uM ATP; 20 mM MgCl₂; 1 mM DTT; 25 mM β-glycerophosphate; 5 mM EGTA; and 1 mM sodium orthovanadate for 1 h at 30° C. The compounds of the inventions solubilized in 100% DMSO were pre-diluted in assay buffer so that the final concentration of DMSO in the reaction was 1%. The kinase reaction was carried out in 100 ul volume on 96 well plates. The kinase reaction was then stopped with the addition of 100 mM EDTA. The entire reaction mix was then transferred to the detection plate, a 96 well Immunosorb plate that had been pre-coated with anti-GST antibody (Amersham). After a 1 hour incubation at room temperature, the detection plate was washed 4 times with TBST (TBS+0.05% Tween 20) and then incubated for another hour at room temperature with anti phospho-MEK1 antibody (Cell Signaling) 1:1000 in 10 mM MOPS 7.5; 150 mM NaCl; 0.05% Tween 20; 0.1% Gelatin; 0.02% NaN₃; and 1% BSA. The detection plate was washed again and incubated for 30 min with DELFIA Europium (Eu) labeled goat anti-rabbit IgG (Perkin-Elmer), 1:4000 in the same buffer used for the primary incubation. After a final wash, Eu detection solution was added to each well and the Eu signal was measured in a Wallac Victor² Multilabel Counter. IC₅₀ calculations were performed using the XLfit software package (IDBS, Guildford, UK). IC₅₀ values for representative compounds according to the invention are listed in Table 1 below.

TABLE 1

| Example | IC₅₀ (μM) |
|---|---|
| 1 | 11 |
| 2 | 23.9 |
| 3 | 0.24 |
| 4 | 0.03 |
| 5 | 0.37 |
| 6 | 0.2 |
| 7 | 0.018 |
| 8 | 0.031 |
| 9 | 0.211 |
| 10 | 0.2 |
| 11 | 0.123 |
| 12 | 1.3 |
| 13 | 0.03 |
| 14 | 0.14 |
| 15 | 0.015 |
| 16 | 0.227 |
| 17 | 0.19 |
| 18 | 0.28 |
| 19 | 0.59 |
| 20 | 0.61 |
| 21 | 0.13 |
| 22 | 0.068 |
| 23 | 0.64 |
| 24 | 0.38 |
| 25 | 0.082 |
| 26 | >40 |
| 27 | 0.074 |
| 28 | 1.1 |
| 29 | 5.2 |
| 30 | 0.13 |
| 31 | 0.11 |
| 32 | >40 |
| 33 | 0.2 |
| 34 | 0.26 |
| 35 | 1.8 |
| 36 | 0.63 |
| 37 | 0.41 |
| 38 | 2.6 |
| 39 | 0.011 |
| 40 | 2.4 |
| 41 | 2.2 |
| 42 | 26.8 |
| 43 | 15.3 |
| 44 | 3.3 |
| 45 | 14.7 |
| 46 | 30 |
| 47 | 0.013 |
| 48 | 1.5 |
| 49 | >40 |
| 50 | 0.2 |
| 51 | 0.11 |
| 52 | 1.5 |
| 53 | 0.54 |
| 54 | 1.3 |
| 55 | 1.8 |
| 56 | 3.4 |
| 57 | 0.65 |
| 58 | 1.1 |
| 59 | 3.8 |
| 60 | >40 |
| 61 | 0.81 |
| 62 | >40 |
| 63 | >40 |
| 64 | >40 |
| 66 | >40 |
| 68 | 11 |
| 69 | 0.42 |
| 70 | 0.036 |
| 71 | 0.4 |
| 72 | 0.024 |
| 73 | >40 |
| 74 | 0.14 |
| 75 | 2.6 |
| 76 | 0.0068 |
| 77 | 0.11 |
| 78 | 0.075 |
| 79 | 0.57 |
| 80 | >40 |
| 81 | >40 |
| 82 | 3.9 |
| 83 | 0.063 |
| 84 | >40 |
| 85 | 0.67 |
| 86 | 1.1 |
| 87 | 0.13 |
| 88 | 0.25 |
| 89 | 0.29 |
| 90 | 1.7 |
| 91 | 0.36 |
| 92 | >40 |
| 93 | >40 |
| 94 | 0.53 |
| 95 | >40 |
| 96 | 0.44 |
| 97 | 0.23 |
| 98 | 1.5 |
| 99 | 24.1 |
| 100 | >40 |
| 101 | 0.0041 |
| 102 | 1.3 |
| 103 | 0.51 |
| 104 | 1.5 |
| 105 | 0.17 |
| 106 | 5.9 |
| 107 | 0.9 |
| 108 | 0.55 |
| 109 | 2.2 |
| 110 | 0.087 |
| 111 | 0.033 |
| 112 | 0.0035 |
| 113 | 0.01 |
| 114 | 0.31 |
| 115 | 0.13 |
| 116 | 0.016 |
| 117 | >40 |
| 118 | 0.11 |
| 119 | 0.35 |
| 120 | 0.024 |
| 121 | 0.114 |
| 122 | 0.58 |
| 123 | 0.54 |
| 124 | >10 |
| 125 | 11 |
| 126 | 3.3 |
| 127 | 0.064 |
| 128 | 0.0032 |
| 129 | 0.014 |
| 130 | 0.26 |
| 131 | 16 |
| 132 | 17.3 |
| 133 | 0.2 |
| 134 | >40 |
| 135 | >40 |
| 136 | 0.064 |
| 137 | 0.028 |
| 138 | 0.027 |
| 139 | 0.099 |
| 140 | 0.02 |
| 141 | 0.12 |
| 142 | 1.8 |
| 143 | 0.75 |
| 144 | 0.92 |
| 145 | 2.1 |
| 146 | 5.3 |
| 147 | 1.6 |
| 148 | 2.2 |
| 149 | 1.5 |
| 150 | 6.4 |
| 151 | 0.61 |
| 152 | 1.3 |
| 153 | 0.22 |
| 154 | 2.5 |
| 155 | 1.5 |
| 156 | 0.42 |
| 157 | 8.5 |
| 158 | >40 |
| 159 | 9.2 |
| 160 | >40 |
| 161 | >40 |
| 162 | >40 |
| 163 | 0.15 |
| 164 | >40 |
| 165 | >40 |
| 166 | >40 |
| 167 | 0.028 |
| 168 | 0.15 |
| 169 | 0.021 |
| 170 | 0.68 |
| 171 | 0.014 |
| 172 | 0.084 |
| 173 | >40 |
| 174 | 3.5 |
| 175 | 0.31 |
| 176 | 4.3 |
| 177 | 0.01 |
| 178 | 0.016 |
| 179 | 3.9 |
| 180 | >40 |
| 181 | 11.3 |
| 182 | 0.018 |
| 183 | 0.022 |
| 184 | 0.11 |
| 185 | 0.047 |
| 186 | 0.045 |
| 187 | 0.13 |
| 188 | 0.059 |
| 189 | 0.1 |
| 190 | 0.32 |
| 191 | 0.064 |
| 192 | 0.17 |
| 193 | 0.41 |
| 194 | 9.7 |
| 195 | 0.032 |
| 196 | 0.066 |
| 198 | 0.87 |
| 199 | 1.4 |
| 200 | 0.068 |
| 201 | 0.0091 |
| 204 | 2.6 |
| 205 | 0.0032 |
| 206 | 7.4 |
| 210 | 1.9 |
| 211 | 0.35 |
| 212 | 0.4 |
| 213 | 1.5 |
| 214 | 0.016 |
| 215 | 0.14 |
| 216 | 0.0059 |
| 217 | 0.0072 |
| 218 | 0.032 |
| 219 | 0.065 |
| 220 | 0.026 |
| 221 | 0.028 |
| 222 | 0.01 |
| 223 | 0.037 |
| 224 | 0.28 |
| 225 | >40 |
| 226 | >40 |
| 227 | >40 |
| 228 | 0.48 |
| 229 | 0.19 |
| 230 | >40 |
| 231 | 19.7 |
| 232 | 24 |
| 233 | >40 |
| 234 | 15.1 |
| 235 | >40 |
| 236 | 0.2 |
| 237 | 24.8 |
| 238 | 8.4 |
| 239 | 0.78 |
| 240 | 0.19 |
| 241 | >40 |
| 242 | >40 |
| 243 | >40 |
| 244 | >40 |
| 245 | 0.91 |
| 246 | 2.4 |
| 247 | 2.5 |
| 248 | 2.5 |
| 249 | 0.38 |
| 250 | 6.8 |
| 251 | 0.65 |
| 252 | 6.1 |
| 253 | 1.4 |
| 254 | 19.9 |
| 255 | >40 |
| 256 | 0.31 |
| 257 | 0.37 |
| 258 | 0.27 |
| 259 | >40 |
| 260 | 0.83 |
| 261 | >40 |
| 262 | 2.2 |
| 263 | 1.6 |
| 264 | 2.5 |
| 265 | 0.021 |
| 266 | 0.013 |
| 267 | 0.1 |
| 268 | 0.012 |
| 269 | 0.016 |
| 270 | 0.1 |
| 271 | 0.054 |
| 272 | 0.047 |
| 273 | 0.083 |
| 274 | .035 |
| 275 | 0.37 |
| 276 | 4.7 |
| 277 | 0.066 |
| 278 | 0.46 |
| 279 | 0.0026 |
| 280 | >40 |
| 281 | 0.042 |
| 282 | 0.023 |
| 283 | 0.13 |
| 284 | 0.02 |
| 286 | 0.19 |
| 287 | 0.34 |
| 288 | >40 |
| 289 | 0.203 |
| 290 | >40 |
| 291 | 0.0072 |
| 292 | 0.0035 |
| 293 | 0.013 |
| 294 | 0.5 |
| 295 | 0.067 |
| 296 | 0.015 |
| 297 | 0.029 |
| 298 | 0.038 |
| 299 | 0.48 |
| 300 | 0.022 |
| 301 | 0.008 |
| 302 | 0.021 |
| 303 | >40 |
| 304 | 0.017 |
| 305 | 0.025 |
| 306 | 0.017 |
| 307 | 0.061 |
| 308 | 0.028 |
| 309 | 7.1 |
| 310 | 0.15 |
| 311 | 0.012 |
| 312 | 9.7 |
| 313 | 0.23 |
| 314 | 0.91 |
| 315 | 0.018 |
| 316 | 0.01 |
| 317 | 0.019 |
| 318 | 0.0057 |
| 319 | 0.21 |
| 320 | 0.0056 |
| 321 | 0.49 |
| 322 | 2.12 |
| 323 | 1.36 |
| 324 | 0.05 |
| 325 | 0.01 |
| 326 | 0.07 |
| 327 | 0.0016 |
| 328 | 0.0082 |
| 329 | 0.0044 |
| 330 | 0.0085 |
| 331 | 0.0082 |
| 332 | 0.039 |
| 333 | 0.0011 |
| 334 | 0.12 |
| 335 | 0.02 |

Additional representative compounds of the invention made according to the methods described herein and their corresponding IC₅₀ values are listed in Table 2 below.

TABLE 2

| | | IC₅₀ |
|---|---|---|
| Example | Compound | (μM) |
| 336 | 6-bromo-4-[(4-phenoxyphenyl) amino]quinoline-3-carbonitrile | 1 |
| 337 | 4-[(3-chloro-4-fluorophenyl)amino]-6-[(2-morpholin-4- | 1 |
| | ylethyl)amino]quinoline-3-carbonitrile | |
| 338 | 6-[(2-morpholin-4-ylethyl)amino]-4-[(4- | 0.15 |
| | phenoxyphenyl)amino]quinoline-3-carbonitrile | |
| 339 | 6-bromo-4-[(4-methoxyphenyl) amino]quinoline-3-carbonitrile | 40 |
| 340 | 4-[(3,4-difluorophenyl)amino]-6-{[(6-methoxypyridin-3- | 0.44 |
| | yl)methyl]amino} quinoline-3-carbonitrile | |
| 341 | 4-[(3,4-difluorophenyl)amino]-6-{[(6-oxo-1,6- | 0.4 |
| | dihydropyridin-3-yl)methyl]amino} quinoline-3-carbonitrile | |
| 342 | 6-[(2-morpholin-4-ylethyl)amino]-4-{[3- | >10 |
| | (trifluoromethyl)phenyl]amino}quinoline-3-carbonitrile | |
| 343 | 4-[(3,4-difluorophenyl)amino]-6-{[(1-methyl-6-oxo-1,6- | 1.04 |
| | dihydropyridin-3-yl)methyl] amino}quinoline-3-carbonitrile | |
| 344 | 4-[(3,4-difluorophenyl)amino]-6-({[6-(2-morpholin-4- | 1.48 |
| | ylethoxy)pyridin-3-yl]methyl} amino)quinoline-3-carbonitrile | |
| 345 | 4-[(3,4-difluorophenyl)amino]-6-({[1-(2-morpholin-4-ylethyl)-6-oxo- | 1.7 |
| | 1,6-dihydropyridin-3-yl]methyl}amino)quinoline-3-carbonitrile | |
| 346 | 6-[(1,3-benzodioxol-5-ylmethyl)amino]-4-[(3- | 0.6 |
| | chlorophenyl)amino]quinoline-3-carbonitrile | |
| 347 | 4-[(3-chlorophenyl)amino]-6-({4-[3-(dimethylamino) | 0.6 |
| | propoxy]benzyl}amino)quinoline-3-carbonitrile | |
| 348 | 4-[(3-chlorophenyl)amino]-6-[(pyridin-3-ylmethyl)amino] | 0.09 |
| | quinoline-3-carbonitrile | |
| 349 | 4-[(3-chlorophenyl)amino]-6-{[4-(methylsulfonyl) | 0.37 |
| | benzyl]amino}quinoline-3-carbonitrile | |
| 350 | 4-[(3-chlorophenyl)amino]-6-[(2-morpholin-4- | 0.69 |
| | ylethyl)amino]quinoline-3-carbonitrile | |
| 351 | 4-[(3-fluorophenyl)amino]-6-[(2-morpholin-4- | 1.9 |
| | ylethyl)amino]quinoline-3-carbonitrile | |
| 352 | 4-[(3-isopropylphenyl)amino]-6-[(2-morpholin-4- | >10 |
| | ylethyl)amino]quinoline-3-carbonitrile | |
| 353 | 4-{[3-chloro-4-(phenylthio)phenyl]amino}-6-[(2- | 0.47 |
| | morpholin-4-ylethyl)amino]quinoline-3-carbonitrile | |
| 354 | 4-{[3-(dimethylamino)phenyl]amino}-6-[(2-morpholin-4- | >10 |
| | ylethyl)amino]quinoline-3-carbonitrile | |
| 355 | 4-[(4-bromophenyl)amino]-6-{(2-morpholin-4- | >10 |
| | ylethyl)amino]quinoline-3-carbonitrile | |
| 356 | 4-(1H-indol-5-ylamino)-6-[(2-morpholin-4- | >10 |
| | ylethyl)amino]quinoline-3-carbonitrile | |
| 357 | 4-[(3-ethynylphenyl)amino]-6-[(2-morpholin-4- | 1.3 |
| | ylethyl)amino]quinoline-3-carbonitrile | |
| 358 | 4-[(3-chloro-4-fluorophenyl)thio]-6-[(2-morpholin-4- | >40 |
| | ylethyl)amino]quinoline-3-carbonitrile | |
| 359 | 6-amino-4-[(3-chlorophenyl)amino]-3-cyano-N,N- | 8.4 |
| | dimethylquinoline-8-carboxamide | |
| 360 | 4-(cyclohexylamino)-6-[(pyridin-2- | 0.21 |
| | ylmethyl)amino]quinoline-3-carbonitrile | |
| 361 | 4-anilino-6-[(pyridin-2-ylmethyl)amino]quinoline-3- | 0.25 |
| | carbonitrile | |
| 362 | 8-bromo-4-[(3-chlorophenyl)amino]-6-nitroquinoline-3- | >10 |
| | carbonitrile | |
| 363 | 6-amino-4-[(1-benzylpiperidin-4-yl)amino]quinoline-3- | >40 |
| | carbonitrile | |
| 364 | 4-[(3-chloro-4-fluorophenyl)amino]-6-[(1-methylpiperidin- | >10 |
| | 4-yl)amino]quinoline-3-carbonitrile | |
| 365 | 6-amino-8-bromo-4-[(3-chlorophenyl)amino]quinoline-3- | 0.39 |
| | carbonitrile | |
| 366 | 4-[(3-chloro-4-fluorophenyl)amino]-6-[(1- | 25.5 |
| | isopropylpiperidin-4-yl)amino]quinoline-3-carbonitrile | |
| 367 | 4-[(1-benzylpiperidin-4-yl)amino]-6-[(2-morpholin-4- | >40 |
| | ylethyl)amino]quinoline-3-carbonitrile | |
| 368 | 4-(3-chloro-4-fluorophenoxy)-6-[(2-morpholin-4- | >40 |
| | ylethyl)amino]quinoline-3-carbonitrile | |
| 369 | 4-[(3-chloro-4-fluorophenyl)amino]-6-{[(2-ethyl-4-methyl- | 0.64 |
| | 1H-imidazol-5-yl)methyl]amino}quinoline-3-carbonitrile | |
| 370 | 4-[(3-chloro-4-fluorophenyl)amino]-6-{[(1,5-dimethyl-1H- | 0.0032 |
| | imidazol-4-yl)methyl]amino}quinoline-3-carbonitrile | |
| 371 | 6-{[3-bromo-4-(2-methoxyethoxy)benzyl]amino}-4-[(3- | 0.38 |
| | chloro-4-fluorophenyl)amino]quinoline-3-carbonitrile | |
| 372 | 8-bromo-4-[(3-chlorophenyl)amino]-6-[(2-morpholin-4- | 0.42 |
| | ylethyl)amino]quinoline-3-carbonitrile | |
| 373 | 4-[(3-chloro-4-fluorophenyl)amino]-6-({[5-methyl-1-(2- | 0.014 |
| | morpholin-4-ylethyl)-1H-imidazol-4- | |
| | yl]methyl}amino)quinoline-3-carbonitrile | |
| 374 | 8-bromo-4-[(3-chlorophenyl)amino]-6-[(pyridin-3- | 0.036 |
| | ylmethyl)amino]quinoline-3-carbonitrile | |
| 375 | 4-[(3-chloro-4-fluorophenyl)amino]-6-{[(3-cyano-4-(2- | 0.082 |
| | methoxyethoxy)benzyl]amino}quinoline-3-carbonitrile | |
| 376 | 4-[(3-chlorophenyl)amino]-3-cyano-N,N-dimethyl-6- | 0.4 |
| | [(pyridin-3-ylmethyl)amino]quinoline-8-carboxamide | |
| 377 | 4-[(3-chloro-4-fluorophenyl)amino]-6-[(1-ethyl-3- | >40 |
| | methylpiperidin-4-yl)amino]quinoline-3-carbonitrile | |
| 378 | 6-[(1-benzylpiperidin-4-yl)amino]-4-[(3-chloro-4- | 1.7 |
| | fluorophenyl)amino]quinoline-3-carbonitrile | |
| 379 | tert-butyl 4-({4-[(3-chloro-4-fluorophenyl)amino]-3- | >40 |
| | cyanoquinolin-6-yl}amino)piperidine-1-carboxylate | |
| 380 | 4-[(3-chloro-4-fluorophenyl)amino]-8-methoxy-6- | 1.1 |
| | [(pyridin-3-ylmethyl)amino]quinoline-3-carbonitrile | |
| 381 | tert-butyl {2-bromo-4-[({4-[(3-chloro-4- | >40 |
| | fluorophenyl)amino]-3-cyanoquinolin-6- | |
| | yl}amino)methyl]phenoxy}acetate | |
| 382 | 4-[(3-chloro-4-fluorophenyl)amino]-6-[(1H-pyrazol-3- | 0.074 |
| | ylmethyl)amino]quinoline-3-carbonitrile | |
| 383 | 4-[(3-chloro-4-fluorophenyl)amino]-8-methoxy-6-[(2- | 5.2 |
| | morpholin-4-ylethyl)amino]quinoline-3-carbonitrile | |
| 384 | 4-[(3-chloro-4-fluorophenyl)amino]-6-{[(1-oxidopyridin-3- | 0.13 |
| | yl)methyl]amino}quinoline-3-carbonitrile | |
| 385 | 4-[(3-chloro-4-fluorophenyl)amino]-6-{[(5-methyl-1H- | 0.11 |
| | pyrazol-3-yl)methyl]amino}quinoline-3-carbonitrile | |
| 386 | 4-[(3-hydroxy-4-methylphenyl)amino]-8-methoxy-6- | >40 |
| | [(pyridin-3-ylmethyl)amino]quinoline-3-carbonitrile | |
| 387 | 4-[(3-bromophenyl)amino]-8-[(dimethylamino)methyl]-6- | >40 |
| | [(2-morpholin-4-ylethyl)amino]quinoline-3-carbonitrile | |
| 388 | 4-[(3-chloro-4-fluorophenyl)amino]-6-{[(1,4-dimethyl-1H- | 0.2 |
| | imidazol-5-yl)methyl]amino}quinoline-3-carbonitrile | |
| 389 | 4-[(3-chloro-4-fluorophenyl)amino]-6-{[(4-chloro-1- | >40 |
| | methyl-1H-pyrazol-3-yl)methyl]amino}quinoline-3- | |
| | carbonitrile | |
| 390 | 6-amino-4-[(4-benzylphenyl)amino]quinoline-3- | 0.043 |
| | carbonitrile | |
| 391 | 4-[(4-benzylphenyl)amino]-6-[(2-morpholin-4- | 0.026 |
| | ylethyl)amino]quinoline-3-carbonitrile | |
| 392 | 6-amino-4-[(3-chloro-4-fluorophenyl)amino]-7- | >40 |
| | methylquinoline-3-carbonitrile | |
| 393 | 6-amino-8-bromo-4-[(3-chloro-4- | 0.52 |
| | fluorophenyl)amino]quinoline-3-carbonitrile | |
| 394 | 6-amino-4-[(3-chloro-4-fluorophenyl)amino]-8- | 5.8 |
| | (trifluoromethyl)quinoline-3-carbonitrile | |
| 395 | 4-[(3-chloro-4-fluorophenyl)amino]-3-cyano-N,N- | >40 |
| | dimethyl-6-nitroquinoline-8-carboxamide | |
| 396 | 6-amino-4-[(3-chloro-4-fluorophenyl)amino]-3-cyano- | 6 |
| | N,N-dimethylquinoline-8-carboxamide | |
| 397 | methyl 3-[({4-[(3-chloro-4-fluorophenyl)amino]-3- | 0.32 |
| | cyanoquinolin-6-yl}amino)methyl]benzoate | |
| 398 | 3-[({4-[(3-chloro-4-fluorophenyl)amino]-3-cyanoquinolin- | 0.18 |
| | 6-yl}amino)methyl]benzoic acid | |
| 399 | 3-[({4-[(3-chloro-4-fluorophenyl)amino]-3-cyanoquinolin- | 0.46 |
| | 6-yl}amino)methyl]benzamide | |
| 400 | 4-(cycloheptylamino)-6-[(1H-imidazol-5- | 0.49 |
| | ylmethyl)amino]quinoline-3-carbonitrile | |
| 401 | 4-(tert-butylamino)-6-[(1H-imidazol-5- | 3.1 |
| | ylmethyl)amino]quinoline-3-carbonitrile | |
| 402 | 6-amino-4-(cyclopentylamino)quinoline-3-carbonitrile | >40 |
| 403 | 4-(tert-butylamino)-6-[(3-cyanobenzyl)amino]quinoline-3- | 11.5 |
| | carbonitrile | |
| 404 | 4-(tert-butylamino)-6-{[(3-(methylsulfonyl)benzyl]amino} | 4.8 |
| | quinoline-3-carbonitrile | |
| 405 | 4-(tert-butylamino)-6-[(1H-pyrazol-5- | >40 |
| | ylmethyl)amino]quinoline-3-carbonitrile | |
| 406 | 4-(tert-butylamino)-6-{[(1-oxidopyridin-2-yl)methyl]amino} | 5.4 |
| | quinoline-3-carbonitrile | |
| 407 | 4-(tert-butylamino)-6-{[(1-methyl-1H-imidazol-2- | 5.1 |
| | yl)methyl]amino}quinoline-3-carbonitrile | |
| 408 | 4-({[4-(tert-butylamino)-3-cyanoquinolin-6- | 21.9 |
| | yl]amino}methyl) benzenesulfonamide | |
| 409 | 6-amino-8-chloro-4-[(3-chloro-4- | 0.92 |
| | fluorophenyl)amino]quinoline-3-carbonitrile | |
| 410 | 4-(cyclopentylamino)-6-{[(1-oxidopyridin-2- | 1.3 |
| | yl)methyl]amino} quinoline-3-carbonitrile | |
| 411 | 4-({[3-cyano-4-(cyclopentylamino)quinolin-6- | 7.3 |
| | yl]amino}methyl)benzenesulfonamide | |
| 412 | 4-(cyclopentylamino)-6-{[(1-methyl-1H-imidazol-2- | 3 |
| | yl)methyl]amino}quinoline-3-carbonitrile | |
| 413 | 4-(cyclopentylamino)-6-[(1H-pyrazol-5-ylmethyl)amino] | 1.6 |
| | quinoline-3-carbonitrile | |
| 414 | 4-(cyclopentylamino)-6-{[3- | 2.9 |
| | (methylsulfonyl)benzyl]amino} quinoline-3-carbonitrile | |
| 415 | 6-(benzylamino)-4-(cyclopentylamino)quinoline-3- | 21.3 |
| | carbonitrile | |
| 416 | 6-[(2-cyanobenzyl)amino]-4- | 1.7 |
| | (cyclopentylamino)quinoline-3-carbonitrile | |
| 417 | 4-(cyclopentylamino)-6-[(1H-imidazol-5-ylmethyl)amino] | 0.3 |
| | quinoline-3-carbonitrile | |
| 418 | 4-(cyclopentylamino)-6-[(pyridin-3- | 2.6 |
| | ylmethyl)amino]quinoline-3-carbonitrile | |
| 419 | 4-(cyclopentylamino)-6-[(2-morpholin-4- | 19.7 |
| | ylethyl)amino]quinoline-3-carbonitrile | |
| 420 | 6-[(3-cyanobenzyl)amino]-4- | 2.2 |
| | (cyclopentylamino)quinoline-3-carbonitrile | |
| 421 | 6-amino-4-[(3-chloro-4-fluorophenyl)amino]-8- | 10.6 |
| | hydroxyquinoline-3-carbonitrile | |
| 422 | 4-[(3-chloro-4-fluorophenyl)amino]-6-{[(1-oxidopyridin-2- | >40 |
| | yl)methyl]amino}-2-oxo-1,2-dihydroquinoline-3- | |
| | carbonitrile | |
| 423 | 4-[(3-chloro-4-fluorophenyl)amino]-6-[(1H-imidazol-5- | >40 |
| | ylmethyl)amino]-2-oxo-1,2-dihydroquinoline-3- | |
| | carbonitrile | |
| 424 | 8-bromo-4-[(3-chloro-4-fluorophenyl)amino]-6-{[4- | >40 |
| | (1,1,2,2-tetrafluoroethoxy)benzyl]amino}quinoline-3- | |
| | carbonitrile | |
| 425 | 6-amino-4-[(3-tert-butyl-1-methyl-1H-pyrazol-5-yl)amino] | >40 |
| | quinoline-3-carbonitrile | |
| 426 | 8-chloro-4-[(3-chloro-4-fluorophenyl)amino]-6-{[(1- | 0.0032 |
| | methyl-1H-imidazol-2-yl)methyl]amino}quinoline-3- | |
| | carbonitrile | |
| 427 | 8-chloro-4-[(3-chloro-4-fluorophenyl)amino]-6-[(1H- | 0.047 |
| | pyrazol-3-ylmethyl)amino]quinoline-3-carbonitrile | |
| 428 | 8-bromo-4-[(3-chloro-4-fluorophenyl)amino]-6-({4-[2,2,2- | >40 |
| | trifluoro-1-hydroxy-1-(trifluoromethyl)ethyl]benzyl}amino) | |
| | quinoline-3-carbonitrile | |
| 429 | 8-chloro-4-[(3-chloro-4-fluorophenyl)amino]-6-{[(5- | 0.1 |
| | methyl-1H-pyrazol-3-yl)methyl]amino}quinoline-3- | |
| | carbonitrile | |
| 430 | 8-chloro-4-[(3-chloro-4-fluorophenyl)amino]-6-{[(1,3- | 0.12 |
| | dimethyl-1H-pyrazol-5-yl)methyl]amino}quinoline-3- | |
| | carbonitrile | |
| 431 | 8-chloro-4-[(3-chloro-4-fluorophenyl)amino]-6-{[(2,5- | 0.22 |
| | dimethyl-1H-imidazol-4-yl)methyl]amino}quinoline-3- | |
| | carbonitrile | |
| 432 | 8-chloro-4-[(3-chloro-4-fluorophenyl)amino]-6-({[2- | 0.26 |
| | (methoxymethyl)-1H-imidazol-4- | |
| | yl]methyl}amino)quinoline-3-carbonitrile | |
| 433 | 8-bromo-4-[(3-chloro-4-fluorophenyl)amino]-6-{[(1- | 0.25 |
| | phenyl-1H-pyrazol-5-yl)methyl]amino}quinoline-3- | |
| | carbonitrile | |
| 434 | 8-bromo-4-[(3-chloro-4-fluorophenyl)amino]-6-{[(2- | >40 |
| | phenyl-2H-1,2,3-triazol-4-yl)methyl]amino}quinoline-3- | |
| | carbonitrile | |
| 435 | 8-bromo-4-[(3-chloro-4-fluorophenyl)amino]-6-({[5- | 0.021 |
| | (trimethylsilyl)-2H-1,2,3-triazol-4- | |
| | yl]methyl}amino)quinoline-3-carbonitrile | |
| 436 | 8-bromo-4-[(3-chloro-4-fluorophenyl)amino]-6-{[(1- | 0.15 |
| | hydroxy-1H-imidazol-5-yl)methyl]amino}quinoline-3- | |
| | carbonitrile | |
| 437 | 4-[(3-chloro-4-fluorophenyl)amino]-8-fluoro-6-{[(1- | 0.2 |
| | hydroxy-1H-imidazol-5-yl)methyl]amino}quinoline-3- | |
| | carbonitrile | |
| 438 | 8-chloro-4-[(3-chloro-4-fluorophenyl)amino]-6-{[(1- | 0.11 |
| | hydroxy-1H-imidazol-5-yl)methyl]amino}quinoline-3- | |
| | carbonitrile | |
| 439 | 8-chloro-4-[(3-chloro-4-fluorophenyl)amino]-6-{[(1- | 0.22 |
| | methyl-1H-imidazol-5-yl)methyl]amino}quinoline-3- | |
| | carbonitrile | |
| 440 | 8-chloro-4-[(3-chloro-4-fluorophenyl)amino]-6-[(1,2,3- | 0.049 |
| | thiadiazol-4-ylmethyl)amino]quinoline-3-carbonitrile | |
| 441 | 8-chloro-4-[(3-chloro-4-fluorophenyl)amino]-6-{[(2- | 1.9 |
| | cyclopentyl-5-methyl-1H-imidazol-4- | |
| | yl)methyl]amino}quinoline-3-carbonitrile | |
| 442 | methyl 2-[({8-chloro-4-[(3-chloro-4-fluorophenyl)amino]- | 3.4 |
| | 3-cyanoquinolin-6-yl}amino)methyl]-1-methyl-1H- | |
| | imidazole-5-carboxylate | |
| 443 | 8-chloro-4-[(3-chloro-4-fluorophenyl)amino]-6-{[(5- | 0.025 |
| | propyl-1H-imidazol-4-yl)methyl]amino}quinoline-3- | |
| | carbonitrile | |
| 444 | 8-chloro-4-[(3-chloro-4-fluorophenyl)amino]-6-{[(5- | 20.6 |
| | methyl-2-pyridin-3-yl-1H-imidazol-4- | |
| | yl)methyl]amino}quinoline-3-carbonitrile | |
| 445 | 8-chloro-4-[(3-chloro-4-fluorophenyl)amino]-6-{[(3- | 1.1 |
| | methoxy-2-thienyl)methyl]amino}quinoline-3-carbonitrile | |
| 446 | 8-bromo-4-(3-chloro-4-fluorophenylamino)-6-((1-methyl- | 0.098 |
| | 1H-pyrazol-5-yl)methylamino)quinoline-3-carbonitrile | |
| 447 | 8-chloro-4-[(3-chloro-4-fluorophenyl)amino]-6-[(2-oxo-2- | 0.87 |
| | phenylethyl)amino]quinoline-3-carbonitrile | |
| 448 | 8-bromo-4-[(3-chloro-4-fluorophenyl)amino]-6-[(4,5,6,7- | 0.37 |
| | tetrahydro-1H-indazol-3-ylmethyl)amino]quinoline-3- | |
| | carbonitrile | |
| 449 | 8-bromo-4-[(3-chloro-4-fluorophenyl)amino]-6-{[(1,5- | 5.5 |
| | dimethyl-1H-pyrazol-3-yl)methyl]amino}quinoline-3- | |
| | carbonitrile | |
| 450 | 8-chloro-4-[(3-chloro-4-fluorophenyl)amino]-6-[(2-methyl- | 2.4 |
| | 2-morpholin-4-ylpropyl)amino]quinoline-3-carbonitrile | |
| 451 | 8-chloro-4-[(3-chloro-4-fluorophenyl)amino]-6-[(2H-1,2,3- | 0.031 |
| | triazol-4-ylmethyl)amino]quinoline-3-carbonitrile | |
| 452 | 4-[(3-chloro-4-fluorophenyl)amino]-6-[(1H-imidazol-5- | 0.051 |
| | ylmethyl)amino]-8-iodoquinoline-3-carbonitrile | |
| 453 | 8-bromo-4-[(3-chloro-4-fluorophenyl)amino]-6-{[(1- | >40 |
| | methyl-5-phenyl-1H-pyrazol-3- | |
| | yl)methyl]amino}quinoline-3-carbonitrile | |
| 454 | 8-bromo-4-[(3-chloro-4-fluorophenyl)amino]-6-{[(5- | 0.33 |
| | phenyl-1H-pyrazol-3-yl)methyl]amino}quinoline-3- | |
| | carbonitrile | |
| 455 | 8-bromo-4-[(3-chloro-4-fluorophenyl)amino]-6-{[(5- | 13.5 |
| | pyridin-4-yl-1H-pyrazol-3-yl)methyl]amino}quinoline-3- | |
| | carbonitrile | |
| 456 | 8-bromo-6-{[(4-bromo-1-methyl-1H-pyrazol-5- | 1 |
| | yl)methyl]amino}-4-[(3-chloro-4- | |
| | fluorophenyl)amino]quinoline-3-carbonitrile | |
| 457 | 4-[(3-chloro-4-fluorophenyl)amino]-6-[(1H-imidazol-5- | 0.17 |
| | ylmethyl)amino]-8-vinylquinoline-3-carbonitrile | |
| 458 | 8-chloro-4-[(3-chloro-4-fluorophenyl)amino]-6-[(2,3- | 1.3 |
| | dihydroxybenzyl)amino]quinoline-3-carbonitrile | |
| 459 | 8-acetyl-4-[(3-chloro-4-fluorophenyl)amino]-6-[(1H- | 0.023 |
| | imidazol-5-ylmethyl)amino]quinoline-3-carbonitrile | |
| 460 | 6-[({1-[(benzyloxy)methyl]-4-[3-(dimethylamino)prop-1- | 2 |
| | yn-1-yl]-1H-imidazol-5-yl}methyl)amino]-8-chloro-4-[(3- | |
| | chloro-4-fluorophenyl)amino]quinoline-3-carbonitrile | |
| 461 | 8-chloro-4-[(3-chloro-4-fluorophenyl)amino]-6-[({4-[3- | 0.13 |
| | (dimethylamino)propyl]-1H-imidazol-5- | |
| | yl}methyl)amino]quinoline-3-carbonitrile | |
| 462 | 4-[(3-chloro-4-fluorophenyl)amino]-8-(2-hydroxyethyl)-6- | 1.2 |
| | [(1H-imidazol-5-ylmethyl)amino]quinoline-3-carbonitrile | |
| 463 | 4-[(3-chloro-4-fluorophenyl)amino]-8-(1-hydroxyethyl)-6- | 0.26 |
| | [(1H-imidazol-5-ylmethyl)amino]quinoline-3-carbonitrile | |
| 464 | 4-[(3-chloro-4-fluorophenyl)amino]-8-(hydroxymethyl)-6- | 0.57 |
| | [(1H-imidazol-5-ylmethyl)amino]quinoline-3-carbonitrile | |
| 465 | N-{8-chloro-4-[(3-chloro-4-fluorophenyl)amino]-3- | 0.051 |
| | cyanoquinolin-6-yl}glycine | |
| 466 | 8-chloro-4-[(3-chloro-4-fluorophenyl)amino]-6-{[(2- | 0.23 |
| | methyl-2H-tetrazol-5-yl)methyl]amino}quinoline-3- | |
| | carbonitrile | |
| 467 | 8-chloro-4-[(3-chloro-4-fluorophenyl)amino]-6-{[2-(2- | 1.2 |
| | methyl-2H-tetrazol-5-yl)ethyl]amino}quinoline-3- | |
| | carbonitrile | |
| 468 | 8-chloro-4-[(3-chloro-4-fluorophenyl)amino]-6-{[2-(4- | 0.36 |
| | methylpiperazin-1-yl)-2-oxoethyl]amino}quinoline-3- | |
| | carbonitrile | |
| 469 | 8-chloro-4-[(3-chloro-4-fluorophenyl)amino]-6-{[2-(1- | 0.17 |
| | methyl-1H-tetrazol-5-yl)ethyl]amino}quinoline-3- | |
| | carbonitrile | |
| 470 | 8-chloro-4-[(3-chloro-4-fluorophenyl)amino]-6-[(2-oxo-2- | 0.42 |
| | piperazin-1-ylethyl)amino]quinoline-3-carbonitrile | |
| 471 | 2-(8-chloro-4-(3-chloro-4-fluorophenylamino)-3- | >40 |
| | cyanoquinolin-6-ylamino)-N-methoxy-N- | |
| | methylacetamide | |
| 472 | 2-(8-chloro-4-(3-chloro-4-fluorophenylamino)-3- | 0.16 |
| | cyanoquinolin-6-ylamino)-N-methoxyacetamide | |
| 473 | 2-(8-chloro-4-(3-chloro-4-fluorophenylamino)-3- | 0.47 |
| | cyanoquinolin-6-ylamino)-N-methylacetamide | |
| 474 | 2-(8-chloro-4-(3-chloro-4-fluorophenylamino)-3- | 1.7 |
| | cyanoquinolin-6-ylamino)-N,N-dimethylacetamide | |
| 475 | ethyl N-{8-chloro-4-[(3-chloro-4-fluorophenyl)amino]-3- | 1.4 |
| | cyanoquinolin-6-yl}glycinate | |
| 476 | 8-chloro-4-[(3-chloro-4-fluorophenyl)amino]-6-{[(5-ethyl- | 0.016 |
| | 1H-imidazol-4-yl)methyl]amino}quinoline-3-carbonitrile | |
| 477 | 6-(bis{[1-(2-morpholin-4-ylethyl)-1H-1,2,3-triazol-4- | >40 |
| | yl]methyl}amino)-8-bromo-4-[(3-chloro-4- | |
| | fluorophenyl)amino]quinoline-3-carbonitrile | |
| 478 | 8-chloro-4-[(3-chloro-4-fluorophenyl)amino]-6-{[(1- | 0.53 |
| | methyl-1H-1,2,4-triazol-3-yl)methyl]amino}quinoline-3- | |
| | carbonitrile | |
| 479 | 6-({[1-(1-adamantyl)-1H-1,2,3-triazol-4-yl]methyl}amino)- | 1.7 |
| | 8-bromo-4-[(3-chloro-4-fluorophenyl)amino]quinoline-3- | |
| | carbonitrile | |
| 480 | 8-bromo-4-[(3-chloro-4-fluorophenyl)amino]-6-[({1-[3- | 0.02 |
| | (dimethylamino)propyl]-1H-1,2,3-triazol-4- | |
| | yl}methyl)amino]quinoline-3-carbonitrile | |
| 481 | 8-bromo-4-[(3-chloro-4-fluorophenyl)amino]-6-[({1-[2- | 0.024 |
| | (dimethylamino)ethyl]-1H-1,2,3-triazol-4- | |
| | yl}methyl)amino]quinoline-3-carbonitrile | |
| 482 | 8-bromo-4-[(3-chloro-4-fluorophenyl)amino]-6-({[1-(2- | 0.16 |
| | morpholin-4-ylethyl)-1H-imidazol-5- | |
| | yl]methyl}amino)quinoline-3-carbonitrile | |
| 483 | 8-chloro-4-[(3-chloro-4-fluorophenyl)amino]-6-[(1H-1,2,4- | 0.1 |
| | triazol-3-ylmethyl)amino]quinoline-3-carbonitrile | |
| 484 | 8-bromo-4-[(3-chloro-4-fluorophenyl)amino]-6-[(1H- | 0.11 |
| | 1,2,4-triazol-3-ylmethyl)amino]quinoline-3-carbonitrile | |
| 485 | 6-{[(1-tert-butyl-1H-1,2,3-triazol-4-yl)methyl]amino}-8- | 0.029 |
| | chloro-4-[(3-chloro-4-fluorophenyl)amino]quinoline-3- | |
| | carbonitrile | |
| 486 | 8-bromo-4-[(3-chloro-4-fluorophenyl)amino]-6-{[(1- | 0.2 |
| | methyl-1H-1,2,4-triazol-3-yl)methyl]amino}quinoline-3- | |
| | carbonitrile | |
| 487 | 8-bromo-4-[(3-chloro-4-fluorophenyl)amino]-6-({[1-(2- | 0.011 |
| | hydroxypropyl)-1H-1,2,3-triazol-4- | |
| | yl]methyl}amino)quinoline-3-carbonitrile | |
| 488 | 8-bromo-4-[(3-chloro-4-fluorophenyl)amino]-6-({[1-(2- | 0.041 |
| | morpholin-4-ylethyl)-1H-imidazol-4- | |
| | yl]methyl}amino)quinoline-3-carbonitrile | |
| 489 | 8-chloro-4-[(3-chloro-4-fluorophenyl)amino]-6-({[1-(2- | 0.062 |
| | morpholin-4-ylethyl)-1H-imidazol-5- | |
| | yl]methyl}amino)quinoline-3-carbonitrile | |
| 490 | 8-chloro-4-[(3-chloro-4-fluorophenyl)amino]-6-({[1-(2- | 0.033 |
| | morpholin-4-ylethyl)-1H-imidazol-4- | |
| | yl]methyl}amino)quinoline-3-carbonitrile | |
| 491 | 8-chloro-4-[(3-chloro-4-fluorophenyl)amino]-6-({[1-(2,2,2- | 0.023 |
| | trifluoroethyl)-1H-1,2,3-triazol-4- | |
| | yl]methyl}amino)quinoline-3-carbonitrile | |
| 492 | 8-chloro-4-[(3-chloro-4-fluorophenyl)amino]-6-{[(1- | 0.12 |
| | phenyl-1H-1,2,3-triazol-4-yl)methyl]amino}quinoline-3- | |
| | carbonitrile | |
| 493 | tert-butyl 4-{4-[({8-bromo-4-[(3-chloro-4- | 0.25 |
| | fluorophenyl)amino]-3-cyanoquinolin-6-yl}amino)methyl]- | |
| | 1H-1,2,3-triazol-1-yl}piperidine-1-carboxylate | |
| 494 | tert-butyl 4-({4-[({8-bromo-4-[(3-chloro-4- | 0.17 |
| | fluorophenyl)amino]-3-cyanoquinolin-6-yl}amino)methyl]- | |
| | 1H-1,2,3-triazol-1-yl}methyl) piperidine-1-carboxylate | |
| 495 | 8-bromo-4-[(3-chloro-4-fluorophenyl)amino]-6-({[1- | 0.0048 |
| | (pyridin-4-ylmethyl)-1H-1,2,3-triazol-4- | |
| | yl]methyl}amino)quinoline-3-carbonitrile | |
| 496 | 8-bromo-4-[(3-chloro-4-fluorophenyl)amino]-6-({[1- | 0.047 |
| | (piperidin-4-ylmethyl)-1H-1,2,3-triazol-4- | |
| | yl]methyl}amino)quinoline-3-carbonitrile | |
| 497 | 8-bromo-6-{[(1-tert-butyl-1H-1,2,3-triazol-4- | 0.061 |
| | yl)methyl]amino}-4-[(3-chloro-4- | |
| | fluorophenyl)amino]quinoline-3-carbonitrile | |
| 498 | 8-bromo-4-[(3-chloro-4-fluorophenyl)amino]-6-({[1-(2,2,2- | 0.1 |
| | trifluoroethyl)-1H-1,2,3-triazol-4- | |
| | yl]methyl}amino)quinoline-3-carbonitrile | |
| 499 | 8-bromo-4-[(3-chloro-4-fluorophenyl)amino]-6-[({1-[(1- | 0.012 |
| | methylpiperidin-4-yl)methyl]-1H-1,2,3-triazol-4- | |
| | yl}methyl)amino]quinoline-3-carbonitrile | |
| 500 | tert-butyl (2-{4-[({8-bromo-4-[(3-chloro-4- | 0.067 |
| | fluorophenyl)amino]-3-cyanoquinolin-6-yl}amino)methyl]- | |
| | 1H-1,2,3-triazol-1-yl}ethyl)methylcarbamate | |
| 501 | di-tert-butyl [({8-bromo-4-[(3-chloro-4- | 4.7 |
| | fluorophenyl)amino]-3-cyanoquinolin-6- | |
| | yl}imino)bis(methylene-1H-1,2,3-triazole-4,1-diylethane- | |
| | 2,1-diyl)]bis(methylcarbamate) | |
| 502 | tert-butyl (2-{4-[({8-chloro-4-[(3-chloro-4- | 0.048 |
| | fluorophenyl)amino]-3-cyanoquinolin-6-yl}amino)methyl]- | |
| | 1H-1,2,3-triazol-1-yl}ethyl)methylcarbamate | |
| 503 | tert-butyl 4-(2-{4-[({8-bromo-4-[(3-chloro-4- | 0.13 |
| | fluorophenyl)amino]-3-cyanoquinolin-6-yl}amino)methyl]- | |
| | 1H-1,2,3-triazol-1-yl}ethyl)piperidine-1-carboxylate | |
| 504 | tert-butyl 4-(2-{4-[({8-chloro-4-[(3-chloro-4- | 0.075 |
| | fluorophenyl)amino]-3-cyanoquinolin-6-yl}amino)methyl]- | |
| | 1H-1,2,3-triazol-1-yl}ethyl)piperidine-1-carboxylate | |
| 505 | 8-bromo-4-[(3-chloro-4-fluorophenyl)amino]-6- | 0.29 |
| | [methyl({1-[(1-methylpiperidin-4-yl)methyl]-1H-1,2,3- | |
| | triazol-4-yl}methyl)amino]quinoline-3-carbonitrile | |
| 506 | 8-bromo-4-[(3-chloro-4-fluorophenyl)amino]-6- | 0.022 |
| | (methyl{[1-(1-methylpiperidin-4-yl)-1H-1,2,3-triazol-4- | |
| | yl]methyl}amino)quinoline-3-carbonitrile | |
| 507 | 8-bromo-4-[(3-chloro-4-fluorophenyl)amino]-6-({[4-(3- | 0.0621 |
| | methoxypropyl)-1H-imidazol-5- | |
| | yl]methyl}amino)quinoline-3-carbonitrile | |
| 508 | 8-bromo-4-[(3-chloro-4-fluorophenyl)amino]-6-[({1-[2- | 0.082 |
| | (methylthio)ethyl]-1H-1,2,3-triazol-4- | |
| | yl}methyl)amino]quinoline-3-carbonitrile | |
| 509 | di-tert-butyl 4,4′-[({8-bromo-4-[(3-chloro-4- | >3 |
| | fluorophenyl)amino]-3-cyanoquinolin-6- | |
| | yl}imino)bis(methylene-1H-1,2,3-triazole-4,1-diylethane- | |
| | 2,1-diyl)]dipiperidine-1-carboxylate | |
| 510 | 8-bromo-4-[(3-chloro-4-fluorophenyl)amino]-6-({[1-(2- | 0.028 |
| | hydroxyethyl)-1H-1,2,3-triazol-4- | |
| | yl]methyl}amino)quinoline-3-carbonitrile | |
| 511 | 8-bromo-4-[(3-chloro-4-fluorophenyl)amino]-6-[({1-[2- | 0.091 |
| | (methylsulfonyl)ethyl]-1H-1,2,3-triazol-4- | |
| | yl}methyl)amino]quinoline-3-carbonitrile | |
| 512 | 8-bromo-4-[(3-chloro-4-fluorophenyl)amino]-6-[({1-[2- | 0.038 |
| | (methylamino)ethyl]-1H-1,2,3-triazol-4- | |
| | yl}methyl)amino]quinoline-3-carbonitrile | |
| 513 | 8-chloro-4-[(3-chloro-4-fluorophenyl)amino]-6-[({1-[2- | 0.029 |
| | (methylamino)ethyl]-1H-1,2,3-triazol-4- | |
| | yl}methyl)amino]quinoline-3-carbonitrile | |
| 514 | 8-chloro-4-[(3-chloro-4-fluorophenyl)amino]-6-[({1-[3- | 0.039 |
| | (dimethylamino)propyl]-1H-1,2,3-triazol-4- | |
| | yl}methyl)amino]quinoline-3-carbonitrile | |
| 515 | 8-chloro-4-[(3-chloro-4-fluorophenyl)amino]-6-({[1-(2- | 0.04 |
| | pyrrolidin-1-ylethyl)-1H-1,2,3-triazol-4- | |
| | yl]methyl}amino)quinoline-3-carbonitrile | |
| 516 | 8-chloro-4-[(3-chloro-4-fluorophenyl)amino]-6-[({1-[2-(1- | 0.029 |
| | methylpyrrolidin-2-yl)ethyl]-1H-1,2,3-triazol-4- | |
| | yl}methyl)amino]quinoline-3-carbonitrile | |
| 517 | 8-bromo-4-[(3-chloro-4-fluorophenyl)amino]-6-({[1-(2- | 0.09 |
| | piperidin-4-ylethyl)-1H-1,2,3-triazol-4- | |
| | yl]methyl}amino)quinoline-3-carbonitrile | |
| 518 | 8-chloro-4-[(3-chloro-4-fluorophenyl)amino]-6-({[1-(2- | 0.045 |
| | piperidin-4-ylethyl)-1H-1,2,3-triazol-4- | |
| | yl]methyl}amino)quinoline-3-carbonitrite | |
| 519 | 8-chloro-4-[(3-chloro-4-fluorophenyl)amino]-6-({[1- | 0.01 |
| | (pyridin-3-ylmethyl)-1H-1,2,3-triazol-4- | |
| | yl]methyl}amino)quinoline-3-carbonitrile | |
| 520 | 8-bromo-4-[(3-chloro-4-fluorophenyl)amino]-6-[({1-[2-(1- | 0.044 |
| | methylpiperidin-4-yl)ethyl]-1H-1,2,3-triazol-4- | |
| | yl}methyl)amino]quinoline-3-carbonitrile | |
| 521 | 8-chloro-4-[(3-chloro-4-fluorophenyl)amino]-6-[({1-[2-(1- | 0.038 |
| | methylpiperidin-4-yl)ethyl]-1H-1,2,3-triazol-4- | |
| | yl}methyl)amino]quinoline-3-carbonitrile | |
| 522 | 8-bromo-4-[(3-chloro-4-fluorophenyl)amino]-6-({[1-(2- | 0.018 |
| | methoxyethyl)-1H-1,2,3-triazol-4- | |
| | yl]methyl}amino)quinoline-3-carbonitrile | |
| 523 | 8-bromo-4-[(3-chloro-4-fluorophenyl)amino]-6-({[1-(3- | 0.018 |
| | hydroxypropyl)-1H-1,2,3-triazol-4- | |
| | yl]methyl}amino)quinoline-3-carbonitrile | |
| 524 | 8-bromo-4-[(3-chloro-4-fluorophenyl)amino]-6-[({1-[2- | 0.053 |
| | (cyclopentylsulfonyl)ethyl]-1H-1,2,3-triazol-4- | |
| | yl}methyl)amino]quinoline-3-carbonitrile | |
| 525 | 8-bromo-4-[(3-chloro-4-fluorophenyl)amino]-6-[({1-[1- | 0.0011 |
| | (methylsulfonyl)piperidin-4-yl]-1H-1,2,3-triazol-4- | |
| | yl}methyl)amino]quinoline-3-carbonitrile | |
| 526 | 8-bromo-4-[(3-chloro-4-fluorophenyl)amino]-6- | 0.28 |
| | [(imidazo[1,2-a]pyridin-3-ylmethyl)amino]quinoline-3- | |
| | carbonitrile | |
| 527 | N-(3-chloro-4-fluorophenyl)-5-{4-[(1H-imidazol-5- | >3 |
| | ylmethyl)amino]phenyl}pyrimidin-4-amine | |
| 528 | 8-bromo-4-[(3-chloro-4-fluorophenyl)amino]-6- | 0.11 |
| | [(imidazo[1,2-a]pyrazin-3-ylmethyl)amino]quinoline-3- | |
| | carbonitrile | |
| 529 | 8-bromo-4-[(3-chloro-4-fluorophenyl)amino]-6-[({1-[2- | 0.043 |
| | (methylsulfonyl)ethyl]-1H-imidazol-4- | |
| | yl}methyl)amino]quinoline-3-carbonitrile | |
| 530 | 8-bromo-4-[(3-chloro-4-fluorophenyl)amino]-6-[({1- | 0.012 |
| | [(methylthio)methyl]-1H-1,2,3-triazol-4- | |
| | yl}methyl)amino]quinoline-3-carbonitrile | |
| 531 | 8-bromo-4-[(3-chloro-4-fluorophenyl)amino]-6-[({1- | 0.033 |
| | [(methylsulfonyl)methyl]-1H-1,2,3-triazol-4- | |
| | yl}methyl)amino]quinoline-3-carbonitrile | |
| 532 | 8-bromo-4-[(3-chloro-4-fluorophenyl)amino]-6-[({1- | 0.12 |
| | [(methylsulfinyl)methyl]-1H-1,2,3-triazol-4- | |
| | yl}methyl)amino]quinoline-3-carbonitrile | |
| 533 | 8-bromo-4-[(3-chloro-4-fluorophenyl)amino]-6-({[1- | 0.0031 |
| | (pyridin-3-ylmethyl)-1H-imidazol-4- | |
| | yl]methyl}amino)quinoline-3-carbonitrile | |
| 534 | 4-[(3-chloro-4-fluorophenyl)amino]-5-{2-[(1H-imidazol-5- | >3 |
| | ylmethyl)amino]phenyl}nicotinonitrile | |
| 535 | N-(3-chloro-4-fluorophenyl)-5-{3-[(1H-imidazol-5- | >3 |
| | ylmethyl)amino]phenyl}pyrimidin-4-amine | |
| 536 | N-(3-chloro-4-fluorophenyl)-5-{2-[(1H-imidazol-5- | >3 |
| | ylmethyl)amino]phenyl}pyrimidin-4-amine | |
| 537 | 8-bromo-4-[(3-chloro-4-fluorophenyl)amino]-6-{[(3- | 0.8 |
| | piperidin-4-yl-1H-pyrazol-5-yl)methyl]amino}quinoline-3- | |
| | carbonitrile | |
| 538 | 8-bromo-4-[(3-chloro-4-fluorophenyl)amino]-6-({[1-(3- | 0.0034 |
| | piperidin-1-ylpropyl)-1H-1,2,3-triazol-4- | |
| | yl]methyl}amino)quinoline-3-carbonitrile | |
| 539 | 8-chloro-4-[(3-chloro-4-fluorophenyl)amino]-6-({[1-(3- | 0.0011 |
| | piperidin-1-ylpropyl)-1H-1,2,3-triazol-4- | |
| | yl]methyl}amino)quinoline-3-carbonitrile | |
| 540 | tert-butyl 3-({4-[({8-bromo-4-[(3-chloro-4- | 0.12 |
| | fluorophenyl)amino]-3-cyanoquinolin-6-yl}amino)methyl]- | |
| | 1H-1,2,3-triazol-1-yl}methyl)piperidine-1-carboxylate | |
| 541 | tert-butyl 3-({4-[({8-chloro-4-[(3-chloro-4- | 0.25 |
| | fluorophenyl)amino]-3-cyanoquinolin-6-yl}amino)methyl]- | |
| | 1H-1,2,3-triazol-1-yl}methyl)piperidine-1-carboxylate | |
| 542 | 8-bromo-4-[(3-chloro-4-fluorophenyl)amino]-6-[({1-[1- | 0.19 |
| | (2,2-dimethylpropyl)piperidin-4-yl]-1H-1,2,3-triazol-4- | |
| | yl}methyl)amino]quinoline-3-carbonitrile | |
| 543 | tert-butyl 2-({4-[({8-chloro-4-[(3-chloro-4- | 0.32 |
| | fluorophenyl)amino]-3-cyanoquinolin-6-yl}amino)methyl]- | |
| | 1H-1,2,3-triazol-1-yl}methyl)pyrrolidine-1-carboxylate | |
| 544 | tert-butyl 2-({4-[({8-bromo-4-[(3-chloro-4- | 0.27 |
| | fluorophenyl)amino]-3-cyanoquinolin-6-yl}amino)methy]- | |
| | 1H-1,2,3-triazol-1-yl}methyl)pyrrolidine-1-carboxylate | |
| 545 | 8-bromo-4-[(3-chloro-4-fluorophenyl)amino]-6-({[1- | 0.033 |
| | (piperidin-3-ylmethyl)-1H-1,2,3-triazol-4- | |
| | yl]methyl}amino)quinoline-3-carbonitrile | |
| 546 | 8-chloro-4-[(3-chloro-4-fluorophenyl)amino]-6-({[1- | 0.048 |
| | (piperidin-3-ylmethyl)-1H-1,2,3-triazol-4- | |
| | yl]methyl}amino)quinoline-3-carbonitrile | |
| 547 | 6-({[1-(1-acetylpiperidin-4-yl)-1H-1,2,3-triazol-4- | 0.0061 |
| | yl]methyl}amino)-8-bromo-4-[(3-chloro-4- | |
| | fluorophenyl)amino]quinoline-3-carbonitrile | |
| 548 | 8-bromo-4-[(3-chloro-4-fluorophenyl)amino]-6-[({1-[2-(1- | 0.034 |
| | methylpyrrolidin-2-yl)ethyl]-1H-imidazol-4- | |
| | yl}methyl)amino]quinoline-3-carbonitrile | |
| 54 | 8-chloro-4-[(3-chloro-4-fluorophenyl)amino]-6-({[1- | 0.0091 |
| | (pyrrolidin-2-ylmethyl)-1H-1,2,3-triazol-4- | |
| | yl]methyl}amino)quinoline-3-carbonitrile | |
| 550 | 8-bromo-4-[(3-chloro-4-fluorophenyl)amino]-6-({[1- | 0.0092 |
| | (pyrrolidin-2-ylmethyl)-1H-1,2,3-triazol-4- | |
| | yl]methyl}amino)quinoline-3-carbonitrile | |
| 551 | 6-{[(1-allyl-1H-imidazol-4-yl)methyl]amino}-8-bromo-4- | 0.0048 |
| | [(3-chloro-4-fluorophenyl)amino]quinoline-3-carbonitrile | |
| 552 | 6-({[1-(2-azepan-1-ylethyl)-1H-1,2,3-triazol-4- | 0.0028 |
| | yl]methyl}amino)-8-chloro-4-[(3-chloro-4- | |
| | fluorophenyl)amino]quinoline-3-carbonitrile | |
| 553 | 8-chloro-4-[(3-chloro-4-fluorophenyl)amino]-6-({[1- | 0.0007 |
| | (pyridin-3-ylmethyl)-1H-imidazol-4- | |
| | yl]methyl}amino)quinoline-3-carbonitrile | |
| 554 | 8-bromo-4-[(3-chloro-4-fluorophenyl)amino]-6-{[(1- | 0.023 |
| | pyridin-4-yl-1H-imidazol-4-yl)methyl]amino}quinoline-3- | |
| | carbonitrile | |
| 555 | 8-bromo-4-[(3-chloro-4-fluorophenyl)amino]-6-[({3-[(2S)- | 0.33 |
| | pyrrolidin-2-yl]-1H-pyrazol-5-yl}methyl)amino]quinoline- | |
| | 3-carbonitrile | |
| 556 | 8-chloro-4-[(3-chloro-4-fluorophenyl)amino]-6-[({1-[2- | 0.013 |
| | (dimethylamino)ethyl]-1H-1,2,3-triazol-4- | |
| | yl}methyl)amino]quinoline-3-carbonitrile | |
| 557 | 8-chloro-4-[(3-chloro-4-fluorophenyl)amino]-6-({[1- | 0.0041 |
| | (pyridin-4-ylmethyl)-1H-1,2,3-triazol-4- | |
| | yl]methyl}amino)quinoline-3-carbonitrile | |
| 558 | 8-bromo-4-[(3-chloro-4-fluorophenyl)amino]-6-({[1-(2- | 0.0034 |
| | pyridin-2-ylethyl)-1H-1,2,3-triazol-4- | |
| | yl]methyl}amino)quinoline-3-carbonitrile | |
| 559 | 8-bromo-4-[(3-chloro-4-fluorophenyl)amino]-6-{[(1- | 0.0055 |
| | pyridin-4-yl-1H-1,2,3-triazol-4-yl)methyl]amino}quinoline- | |
| | 3-carbonitrile | |
| 560 | 8-chloro-4-[(3-chloro-4-fluorophenyl)amino]-6-{[(1- | 0.0032 |
| | pyridin-4-yl-1H-1,2,3-triazol-4-yl)methyl]amino}quinoline- | |
| | 3-carbonitrile | |
| 561 | 6-{[(1-allyl-1H-1,2,3-triazol-4-yl)methyl]amino}-8-bromo- | 0.02 |
| | 4-[(3-chloro-4-fluorophenyl)amino]quinoline-3- | |
| | carbonitrile | |
| 562 | 8-chloro-4-[(3-chloro-4-fluorophenyl)amino]-6-({[1-(2- | 0.02 |
| | methoxyethyl)-1H-1,2,3-triazol-4- | |
| | yl]methyl}amino)quinoline-3-carbonitrile | |
| 563 | 8-chloro-4-[(3-chloro-4-fluorophenyl)amino]-6-({[1-(2- | 0.025 |
| | pyridin-2-ylethyl)-1H-1,2,3-triazol-4- | |
| | yl]methyl}amino)quinoline-3-carbonitrile | |
| 564 | 8-chloro-4-[(3-chloro-4-fluorophenyl)amino]-6-[({1-[2-(2- | 0.021 |
| | methoxyethoxy)ethyl]-1H-1,2,3-triazol-4- | |
| | yl}methyl)amino]quinoline-3-carbonitrile | |
| 565 | 8-bromo-4-[(3-chloro-4-fluorophenyl)amino]-6-[({1-[2-(2- | 0.1 |
| | methoxyethoxy)ethyl]-1H-1,2,3-triazol-4- | |
| | yl}methyl)amino]quinoline-3-carbonitrile | |
| 566 | 8-bromo-4-[(3-chloro-4-fluorophenyl)amino]-6-[({1-[(1- | 0.04 |
| | ethylpiperidin-4-yl)methyl]-1H-1,2,3-triazol-4- | |
| | yl}methyl)amino]quinoline-3-carbonitrile | |
| 567 | 8-bromo-4-[(3-chloro-4-fluorophenyl)amino]-6-[({1-[(1- | 0.029 |
| | isopropylpiperidin-4-yl)methyl]-1H-1,2,3-triazol-4- | |
| | yl}methyl)amino]quinoline-3-carbonitrile | |
| 568 | 8-bromo-4-[(3-chloro-4-fluorophenyl)amino]-6-[({1-[2-(1- | 0.064 |
| | ethylpiperidin-4-yl)ethyl]-1H-1,2,3-triazol-4- | |
| | yl}methyl)amino]quinoline-3-carbonitrile | |
| 569 | 8-bromo-4-[(3-chloro-4-fluorophenyl)amino]-6-[({1-[2-(1- | 0.057 |
| | isopropylpiperidin-4-yl)ethyl]-1H-1,2,3-triazol-4- | |
| | yl}methyl)amino]quinoline-3-carbonitrile | |
| 570 | 8-chloro-4-[(3-chloro-4-fluorophenyl)amino]-6-({[1- | 0.014 |
| | (tetrahydro-2H-pyran-4-yl)-1H-1,2,3-triazol-4- | |
| | yl]methyl}amino)quinoline-3-carbonitrile | |
| 571 | 8-bromo-4-[(3-chloro-4-fluorophenyl)amino]-6-[({4-[2- | 0.24 |
| | (dimethylamino)ethyl]-1H-imidazol-5- | |
| | yl}methyl)amino]quinoline-3-carbonitrile | |
| 572 | 8-chloro-4-[(3-chloro-4-fluorophenyl)amino]-6-{[(1-{2-[2- | 0.091 |
| | (2-hydroxyethoxy)ethoxy]ethyl}-1H-1,2,3-triazol-4- | |
| | yl)methyl]amino}quinoline-3-carbonitrile | |
| 573 | 8-bromo-4-[(3-chloro-4-fluorophenyl)amino]-6-{[(1-{2-[2- | 0.14 |
| | (2-hydroxyethoxy)ethoxy]ethyl}-1H-1,2,3-triazol-4- | |
| | yl)methyl]amino}quinoline-3-carbonitrile | |
| 574 | 8-chloro-4-[(3-chloro-4-fluorophenyl)amino]-6-[({1-[2- | 0.07 |
| | (dimethylamino)-1-phenylethyl]-1H-1,2,3-triazol-4- | |
| | yl}methyl)amino]quinoline-3-carbonitrile | |
| 575 | 8-bromo-4-[(3-chloro-4-fluorophenyl)amino]-6-[({1-[2- | 0.093 |
| | (dimethylamino)-1-phenylethyl]-1H-1,2,3-triazol-4- | |
| | yl}methyl)amino]quinoline-3-carbonitrile | |
| 576 | 8-chloro-4-[(3-chloro-4-fluorophenyl)amino]-6-({[1-(1- | 0.016 |
| | oxidopyridin-4-yl)-1H-1,2,3-triazol-4- | |
| | yl]methyl}amino)quinoline-3-carbonitrile | |
| 577 | 8-bromo-4-[(3-chloro-4-fluorophenyl)amino]-6-({[1-(1- | 0.015 |
| | oxidopyridin-4-yl)-1H-1,2,3-triazol-4- | |
| | yl]methyl}amino)quinoline-3-carbonitrile | |
| 578 | 8-chloro-4-[(3-chloro-4-fluorophenyl)amino]-6-({[1-(2- | 0.32 |
| | piperidin-1-ylpyrimidin-4-yl)-1H-1,2,3-triazol-4- | |
| | yl]methyl}amino)quinoline-3-carbonitrile | |
| 579 | 8-bromo-4-[(3-chloro-4-fluorophenyl)amino]-6-({[1- | 0.033 |
| | (pyridin-2-ylmethyl)-1H-1,2,3-triazol-4- | |
| | yl]methyl}amino)quinoline-3-carbonitrile | |
| 580 | 8-chloro-4-[(3-chloro-4-fluorophenyl)amino]-6-({[1- | 0.0021 |
| | (pyridin-2-ylmethyl)-1H-1,2,3-triazol-4- | |
| | yl]methyl}amino)quinoline-3-carbonitrile | |
| 581 | 8-chloro-4-[(3-chloro-4-fluorophenyl)amino]-6-({[1-(3- | 0.0036 |
| | hydroxypropyl)-1H-1,2,3-triazol-4- | |
| | yl]methyl}amino)quinoline-3-carbonitrile | |
| 582 | 3-{4-[({8-chloro-4-[(3-chloro-4-fluorophenyl)amino]-3- | 0.016 |
| | cyanoquinolin-6-yl}amino)methyl]-1H-1,2,3-triazol-1- | |
| | yl}propyl dimethylcarbamate | |
| 583 | 8-chloro-4-[(3-chloro-4-fluorophenyl)amino]-6-[({1-[4- | >3 |
| | (trifluoromethyl)benzyl]-1H-1,2,3-triazol-4- | |
| | yl}methyl)amino]quinoline-3-carbonitrile | |
| 584 | 3-{4-[({8-bromo-4-[(3-chloro-4-fluorophenyl)amino]-3- | 0.0035 |
| | cyanoquinolin-6-yl}amino)methyl]-1H-1,2,3-triazol-1- | |
| | yl}propyl dimethylcarbamate | |
| 585 | 8-chloro-4-[(3-chloro-4-fluorophenyl)amino]-6-({[1- | 0.79 |
| | (cyclohexylmethyl)-1H-1,2,3-triazol-4- | |
| | yl]methyl}amino)quinoline-3-carbonitrile | |
| 586 | 8-chloro-4-[(3-chloro-4-fluorophenyl)amino]-6-({[1-(1- | 0.096 |
| | phenylethyl)-1H-1,2,3-triazol-4- | |
| | yl]methyl}amino)quinoline-3-carbonitrile | |
| 587 | 8-bromo-4-[(3-chloro-4-fluorophenyl)amino]-6-[({1-[3- | 0.0067 |
| | (dimethylamino)-1-pyridin-3-ylpropyl]-1H-1,2,3-triazol-4- | |
| | yl}methyl)amino]quinoline-3-carbonitrile | |
| 588 | 8-chloro-4-[(3-chloro-4-fluorophenyl)amino]-6-[({1-[3- | 0.036 |
| | (dimethylamino)-1-pyridin-3-ylpropyl]-1H-1,2,3-triazol-4- | |
| | yl}methyl)amino]quinoline-3-carbonitrile | |
| 589 | tert-butyl 4-(3-{4-[({8-bromo-4-[(3-chloro-4- | 0.52 |
| | fluorophenyl)amino]-3-cyanoquinolin-6-yl}amino)methyl]- | |
| | 1H-1,2,3-triazol-1-yl}propyl)-1,4-diazepane-1- | |
| | carboxylate | |
| 590 | tert-butyl 4-(3-{4-[({8-chloro-4-[(3-chloro-4- | 0.12 |
| | fluorophenyl)amino]-3-cyanoquinolin-6-yl}amino)methyl]- | |
| | 1H-1,2,3-triazol-1-yl}propyl)-1,4-diazepane-1- | |
| | carboxylate | |
| 591 | tert-butyl 4-(3-{4-[({8-bromo-4-[(3-chloro-4- | 1.3 |
| | fluorophenyl)amino]-3-cyanoquinolin-6-yl}amino)methyl]- | |
| | 1H-1,2,3-triazol-1-yl}propyl)piperazine-1-carboxylate | |
| 592 | tert-butyl 4-(3-{4-[({8-chloro-4-[(3-chloro-4- | 0.23 |
| | fluorophenyl)amino]-3-cyanoquinolin-6-yl}amino)methyl]- | |
| | 1H-1,2,3-triazol-1-yl}propyl)piperazine-1-carboxylate | |
| 593 | 8-chloro-4-[(3-chloro-4-fluorophenyl)amino]-6-{[(1- | 0.0076 |
| | pyridin-3-yl-1H-1,2,3-triazol-4-yl)methyl]amino}quinoline- | |
| | 3-carbonitrile | |
| 594 | 8-bromo-4-[(3-chloro-4-fluorophenyl)amino]-6-[({1-[3- | 0.043 |
| | (1,4-diazepan-1-yl)propyl]-1H-1,2,3-triazol-4- | |
| | yl}methyl)amino]quinoline-3-carbonitrile | |
| 595 | 8-chloro-4-[(3-chloro-4-fluorophenyl)amino]-6-[({1-[3- | 0.074 |
| | (1,4-diazepan-1-yl)propyl]-1H-1,2,3-triazol-4- | |
| | yl}methyl)amino]quinoline-3-carbonitrile | |
| 596 | 8-bromo-4-[(3-chloro-4-fluorophenyl)amino]-6-({[1-(3- | 0.11 |
| | piperazin-1-ylpropyl)-1H-1,2,3-triazol-4- | |
| | yl]methyl}amino)quinoline-3-carbonitrile | |
| 597 | 8-chloro-4-[(3-chloro-4-fluorophenyl)amino]-6-({[1-(3- | 0.16 |
| | piperazin-1-ylpropyl)-1H-1,2,3-triazol-4- | |
| | yl]methyl}amino)quinoline-3-carbonitrile | |
| 598 | 2-(8-chloro-4-(3-chloro-4-fluorophenylamino)-3- | 0.2 |
| | cyanoquinolin-6-ylamino)-N-(pyridin-3- | |
| | ylmethyl)acetamide | |
| 599 | 2-{4-[({8-chloro-4-[(3-chloro-4-fluorophenyl)amino]-3- | 0.0059 |
| | cyanoquinolin-6-yl}amino)methyl]-1H-1,2,3-triazol-1- | |
| | yl}ethyl carbamate | |
| 600 | 2-(8-chloro-4-(3-chloro-4-fluorophenylamino)-3- | 0.048 |
| | cyanoquinolin-6-ylamino)-N-(pyridin-4- | |
| | ylmethyl)acetamide | |
| 601 | 2-(8-chloro-4-(3-chloro-4-fluorophenylamino)-3- | 0.11 |
| | cyanoquinolin-6-ylamino)-N-(pyridin-3-yl)acetamide | |
| 602 | 2-(8-chloro-4-(3-chloro-4-fluorophenylamino)-3- | 2.4 |
| | cyanoquinolin-6-ylamino)-N-(pyridin-4-yl)acetamide | |
| 603 | 2-(8-chloro-4-(3-chloro-4-fluorophenylamino)-3- | >3 |
| | cyanoquinolin-6-ylamino)-N-(3- | |
| | (dimethylamino)propyl)acetamide | |
| 604 | 2-(8-chloro-4-(3-chloro-4-fluorophenylamino)-3- | >3 |
| | cyanoquinolin-6-ylamino)-N-(3- | |
| | morpholinopropyl)acetamide | |
| 605 | 2-(8-chloro-4-(3-chloro-4-fluorophenylamino)-3- | 1.6 |
| | cyanoquinolin-6-ylamino)-N-(2- | |
| | (dimethylamino)ethyl)acetamide | |
| 606 | 2-(8-chloro-4-(3-chloro-4-fluorophenylamino)-3- | 0.23 |
| | cyanoquinolin-6-ylamino)-N-(3- | |
| | methoxypropyl)acetamide | |
| 607 | 2-(8-chloro-4-(3-chloro-4-fluorophenylamino)-3- | 0.33 |
| | cyanoquinolin-6-ylamino)-N-(pyridin-2- | |
| | ylmethyl)acetamide | |
| 608 | 2-(8-chloro-4-(3-chloro-4-fluorophenylamino)-3- | 0.53 |
| | cyanoquinolin-6-ylamino)-N-(2- | |
| | morpholinoethyl)acetamide | |
| 609 | 8-chloro-4-[(3-chloro-4-fluorophenyl)amino]-6-{([1-{3- | 0.0018 |
| | [cyclohexyl(methyl)amino]propyl}-1H-1,2,3-triazol-4- | |
| | yl)methyl]amino}quinoline-3-carbonitrile | |
| 610 | 8-chloro-4-[(3-chloro-4-fluorophenyl)amino]-6-[({1-[3- | 0.15 |
| | (3,5-dimethylpiperidin-1-yl)propyl]-1H-1,2,3-triazol-4- | |
| | yl}methyl)amino]quinoline-3-carbonitrile | |
| 611 | 8-bromo-4-[(3-chloro-4-fluorophenyl)amino]-6- | >3 |
| | {methyl[(1-methyl-1H-imidazol-4- | |
| | yl)methyl]amino}quinoline-3-carbonitrile | |
| 612 | 2-(8-chloro-4-(3-chloro-4-fluorophenylamino)-3- | >3 |
| | cyanoquinolin-6-ylamino)-N-(2-(1-methylpyrrolidin-2- | |
| | yl)ethyl)acetamide | |
| 613 | 2-{4-[({8-chloro-4-[(3-chloro-4-fluorophenyl)amino]-3- | 0.00054 |
| | cyanoquinolin-6-yl}amino)methyl]-1H-1,2,3-triazol-1-yl}- | |
| | N,N-dimethylacetamide | |
| 614 | 3-{4-[({8-chloro-4-[(3-chloro-4-fluorophenyl)amino]-3- | 0.01 |
| | cyanoquinolin-6-yl}amino)methyl]-1H-1,2,3-triazol-1-yl}- | |
| | N,N-dimethylpropanamide | |
| 615 | 8-chloro-4-[(3-chloro-4-fluorophenyl)amino]-6-[({1-[2- | 0.0061 |
| | (1,3-dimethyl-2-oxoimidazolidin-4-yl)ethyl]-1H-1,2,3- | |
| | triazol-4-yl}methyl)amino]quinoline-3-carbonitrile | |
| 616 | 8-chloro-4-[(3-chloro-4-fluorophenyl)amino]-6-[({1-[2-(1- | 0.025 |
| | ethyl-2-oxo-3-phenylimidazolidin-4-yl)ethyl]-1H-1,2,3- | |
| | triazol-4-yl}methyl)amino]quinoline-3-carbonitrile | |
| 617 | 8-chloro-4-[(3-chloro-4-fluorophenyl)amino]-6-[({1-[2-(1- | 0.13 |
| | ethyl-3-phenyl-2-thioxoimidazolidin-4-yl)ethyl]-1H-1,2,3- | |
| | triazol-4-yl}methyl)amino]quinoline-3-carbonitrile | |
| 618 | tert-butyl (2S)-2-[({8-chloro-4-[(3-chloro-4- | >3 |
| | fluorophenyl)amino]-3-cyanoquinolin-6- | |
| | yl}amino)methyl]pyrrolidine-1-carboxylate | |
| 619 | 8-chloro-4-[(3-chloro-4-fluorophenyl)amino]-6-{[(2S)- | >3 |
| | pyrrolidin-2-ylmethyl]amino}quinoline-3-carbonitrile | |
| 620 | 8-bromo-4-[(3-chloro-4-fluorophenyl)amino]-6-({[1- | 0.034 |
| | (pyridin-2-ylmethyl)-1H-imidazol-4- | |
| | yl]methyl}amino)quinoline-3-carbonitrile | |
| 621 | 8-chloro-4-[(3-chloro-4-fluorophenyl)amino]-6-({[1- | 0.041 |
| | (pyridin-2-ylmethyl)-1H-imidazol-4- | |
| | yl]methyl}amino)quinoline-3-carbonitrile | |
| 622 | 8-chloro-4-[(3-chloro-4-fluorophenyl)amino]-6-({[1- | 0.0053 |
| | (pyridin-4-ylmethyl)-1H-imidazol-4- | |
| | yl]methyl}amino)quinoline-3-carbonitrile | |
| 623 | tert-butyl (2S)-2-{[{8-chloro-4-[(3-chloro-4- | >3 |
| | fluorophenyl)amino]-3-cyanoquinolin-6- | |
| | yl}(ethyl)amino]methyl}pyrrolidine-1-carboxylate | |
| 624 | 8-bromo-4-[(3-chloro-4-fluorophenyl)amino]-6-[({1-[3-(4- | 0.07 |
| | methyl-1,4-diazepan-1-yl)propyl]-1H-1,2,3-triazol-4- | |
| | yl}methyl)amino]quinoline-3-carbonitrile | |
| 625 | 8-chloro-4-[(3-chloro-4-fluorophenyl)amino]-6-[({1-[3-(4- | 0.048 |
| | methyl-1,4-diazepan-1-yl)propyl]-1H-1,2,3-triazol-4- | |
| | yl}methyl)amino]quinoline-3-carbonitrile | |
| 626 | 8-bromo-4-[(3-chloro-4-fluorophenyl)amino]-6-[({1-[3-(4- | 0.029 |
| | methylpiperazin-1-yl)propyl]-1H-1,2,3-triazol-4- | |
| | yl}methyl)amino]quinoline-3-carbonitrile | |
| 627 | 8-chloro-4-[(3-chloro-4-fluorophenyl)amino]-6-[({1-[3-(4- | 0.039 |
| | methylpiperazin-1-yl)propyl]-1H-1,2,3-triazol-4- | |
| | yl}methyl)amino]quinoline-3-carbonitrile | |
| 628 | 8-chloro-6-[(2-chlorocyclopentyl)amino]-4-[(3-chloro-4- | >3 |
| | fluorophenyl)amino]quinoline-3-carbonitrile | |
| 629 | 6-(2-azidocyclopentylamino)-8-chloro-4-(3-chloro-4- | 0.42 |
| | fluorophenylamino)quinoline-3-carbonitrile | |
| 630 | N-{8-bromo-4-[(3-chloro-4-fluorophenyl)amino]-3- | 0.14 |
| | cyanoquinolin-6-yl}glycine | |
| 631 | 8-chloro-4-[(3-chloro-4-fluorophenyl)amino]-6-[(2,3- | 0.59 |
| | dihydroxypropyl)amino]quinoline-3-carbonitrile | |
| 632 | 8-bromo-4-[(3-chloro-4-fluorophenyl)amino]-6-{[(1- | 0.23 |
| | cyclopentyl-1H-imidazol-4-yl)methyl]amino}quinoline-3- | |
| | carbonitrile | |
| 633 | 8-bromo-4-[(3-chloro-4-fluorophenyl)amino]-6-{[(1- | >3 |
| | isopropyl-1H-imidazol-4-yl)methyl]amino}quinoline-3- | |
| | carbonitrile | |
| 634 | N-(4-{4-[({8-chloro-4-[(3-chloro-4-fluorophenyl)amino]-3- | 0.035 |
| | cyanoquinolin-6-yl}amino)methyl]-1H-1,2,3-triazol-1- | |
| | yl}phenyl)acetamide | |
| 635 | 8-chloro-4-[(3-chloro-4-fluorophenyl)amino]-6-({2-[4-(2- | 0.067 |
| | hydroxyethyl)-1H-1,2,3-triazol-1- | |
| | yl]cyclopentyl}amino)quinoline-3-carbonitrile | |
| 636 | 8-chloro-4-[(3-chloro-4-fluorophenyl)amino]-6-[(2-{4- | >3 |
| | [(dimethylamino)methyl]-1H-1,2,3-triazol-1- | |
| | yl}cyclopentyl)amino]quinoline-3-carbonitrile | |
| 637 | 8-chloro-4-[(3-chloro-4-fluorophenyl)amino]-6-[(3- | >3 |
| | hydroxy-2-pyridin-4-ylpropyl)amino]quinoline-3- | |
| | carbonitrile | |
| 638 | 6-[({1-[(benzyloxy)methyl]-1H-1,2,3-triazol-4- | >3 |
| | yl}methyl)amino]-8-bromo-4-[(3-chloro-4- | |
| | fluorophenyl)amino]quinoline-3-carbonitrile | |
| 639 | 8-chloro-4-[(3-chloro-4-fluorophenyl)amino]-6-[(2-pyridin- | 0.2 |
| | 4-ylpropyl)amino]quinoline-3-carbonitrile | |
| 640 | 8-chloro-4-[(3-chloro-4-fluorophenyl)amino]-6- | >3 |
| | {ethyl[(2S)-pyrrolidin-2-ylmethyl]amino}quinoline-3- | |
| | carbonitrile | |
| 641 | 8-chloro-4-[(3-chloro-4-fluorophenyl)amino]-6-({[(2S)-1- | 0.33 |
| | ethylpyrrolidin-2-yl]methyl}amino)quinoline-3-carbonitrile | |
| 642 | 6-({[(2S)-1-acetylpyrrolidin-2-yl]methyl}amino)-8-chloro- | 0.054 |
| | 4-[(3-chloro-4-fluorophenyl)amino]quinoline-3- | |
| | carbonitrile | |
| 643 | 8-chloro-4-[(3-chloro-4-fluorophenyl)amino]-6- | 1.2 |
| | (ethyl{[(2S)-1-ethylpyrrolidin-2- | |
| | yl]methyl}amino)quinoline-3-carbonitrile | |
| 644 | 6-[{[(2S)-1-acetylpyrrolidin-2-yl]methyl}(ethyl)amino]-8- | >3 |
| | chloro-4-[(3-chloro-4-fluorophenyl)amino]quinoline-3- | |
| | carbonitrile | |
| 645 | 6-[({1-[(benzyloxy)methyl]-1H-1,2,3-triazol-4- | >3 |
| | yl}methyl)amino]-8-chloro-4-[(3-chloro-4- | |
| | fluorophenyl)amino]quinoline-3-carbonitrile | |
| 646 | 8-chloro-4-[(3-chloro-4-fluorophenyl)amino]-6-({2- | >3 |
| | [(dimethylamino)methyl]cyclohexyl}amino)quinoline-3- | |
| | carbonitrile | |
| 647 | 2-{4-[({8-chloro-4-[(3-chloro-4-fluorophenyl)amino]-3- | 0.0035 |
| | cyanoquinolin-6-yl}amino)methyl]-1H-1,2,3-triazol-1-yl}- | |
| | N-isopropylacetamide | |
| 648 | 6-({[1-(2-azepan-1-yl-2-oxoethyl)-1H-1,2,3-triazol-4- | 0.0046 |
| | yl]methyl}amino)-8-chloro-4-[(3-chloro-4- | |
| | fluorophenyl)amino]quinoline-3-carbonitrile | |
| 649 | 6-({[1-(2-azocan-1-yl-2-oxoethyl)-1H-1,2,3-triazol-4- | 0.0053 |
| | yl]methyl}amino)-8-chloro-4-[(3-chloro-4- | |
| | fluorophenyl)amino]quinoline-3-carbonitrile | |
| 650 | 2-{4-[({8-chloro-4-[(3-chloro-4-fluorophenyl)amino]-3- | 0.015 |
| | cyanoquinolin-6-yl}amino)methyl]-1H-1,2,3-triazol-1-yl}- | |
| | N-(2-methoxyethyl)acetamide | |
| 651 | 2-{4-[({8-chloro-4-[(3-chloro-4-fluorophenyl)amino]-3- | 0.0069 |
| | cyanoquinolin-6-yl}amino)methyl]-1H-1,2,3-triazol-1-yl}- | |
| | N-[2-(1-methylpyrrolidin-2-yl)ethyl]acetamide | |
| 652 | 2-{4-[({8-chloro-4-[(3-chloro-4-fluorophenyl)amino]-3- | 0.0068 |
| | cyanoquinolin-6-yl}amino)methyl]-1H-1,2,3-triazol-1-yl}- | |
| | N-[2-(dimethylamino)ethyl]acetamide | |
| 653 | 2-{4-[({8-chloro-4-[(3-chloro-4-fluorophenyl)amino]-3- | 0.0008 |
| | cyanoquinolin-6-yl}amino)methyl]-1H-1,2,3-triazol-1-yl}- | |
| | N-(pyridin-3-ylmethyl)acetamide | |
| 654 | 2-{4-[({8-chloro-4-[(3-chloro-4-fluorophenyl)amino]-3- | 0.014 |
| | cyanoquinolin-6-yl}amino)methyl]-1H-1,2,3-triazol-1-yl}- | |
| | N-(2-morpholin-4-ylethyl)acetamide | |
| 655 | 8-chloro-4-[(3-chloro-4-fluorophenyl)amino]-6-({[(4S)- | 0.2 |
| | 2,2-dimethyl-1,3-dioxolan-4-yl]methyl}amino)quinoline-3- | |
| | carbonitrile | |
| 656 | tert-butyl 4-(4-(3-chloro-4-fluorophenylamino)-3- | >40 |
| | cyanoquinolin-6-ylamino)piperidine-1-carboxylate | |
| 657 | 6-(1-benzylpiperidin-4-ylamino)-4-(3-chloro-4- | 1.7 |
| | fluorophenylamino)quinoline-3-carbonitrile | |
| 658 | 4-(3-chloro-4-fluorophenylamino)-6-(1-isopropylpiperidin- | 25.5 |
| | 4-ylamino)quinoline-3-carbonitrile | |
| 659 | 4-(3-chloro-4-fluorophenylamino)-6-(1-ethyl-3- | >40 |
| | methylpiperidin-4-ylamino)quinoline-3-carbonitrile | |
| 660 | N-(8-bromo-4-(3-chloro-4-fluorophenylamino)-3- | 4.7 |
| | cyanoquinolin-6-yl)acetamide | |
| 661 | 6-({[1-(1-adamantyl)-1H-1,2,3-triazol-4-yl]methyl}amino)- | 1.7 |
| | 8-bromo-4-[(3-chloro-4-fluorophenyl)amino]quinoline-3- | |
| | carbonitrile | |
| 662 | 8-bromo-4-[(3-chloro-4-fluorophenyl)amino]-6-[({1-[3- | 0.02 |
| | (dimethylamino)propyl]-1H-1,2,3-triazol-4- | |
| | yl}methyl)amino]quinoline-3-carbonitrile | |
| 663 | 8-bromo-4-[(3-chloro-4-fluorophenyl)amino]-6-[({1-[2- | 0.024 |
| | (dimethylamino)ethyl]-1H-1,2,3-triazol-4- | |
| | yl}methyl)amino]quinoline-3-carbonitrile | |
| 664 | 8-bromo-4-[(3-chloro-4-fluorophenyl)amino]-6-{[(1- | 0.2 |
| | methyl-1H-1,2,4-triazol-3-yl)methyl]amino}quinoline-3- | |
| | carbonitrile | |
| 665 | 6-{[(1-tert-butyl-1H-1,2,3-triazol-4-yl)methyl]amino}-8- | 0/029 |
| | chloro-4-[(3-chloro-4-fluorophenyl)amino]quinoline-3- | |
| | carbonitrile | |
| 666 | 8-chloro-4-[(3-chloro-4-fluorophenyl)amino]-6-[(1H-1,2,4- | 0.1 |
| | triazol-3-ylmethyl)amino]quinoline-3-carbonitrile | |
| 667 | 8-bromo-4-[(3-chloro-4-fluorophenyl)amino]-6-[(1H- | 0.11 |
| | 1,2,4-triazol-3-ylmethyl)amino]quinoline-3-carbonitrile | |
| 668 | 8-bromo-4-[(3-chloro-4-fluorophenyl)amino]-6-({[1-(2- | 0.16 |
| | morpholin-4-ylethyl)-1H-imidazol-5- | |
| | yl]methyl}amino)quinoline-3-carbonitrile | |
| 669 | 8-bromo-4-[(3-chloro-4-fluorophenyl)amino]-6-({[1-(2- | 0.041 |
| | morpholin-4-ylethyl)-1H-imidazol-4- | |
| | yl]methyl}amino)quinoline-3-carbonitrile | |
| 670 | 8-chloro-4-[(3-chloro-4-fluorophenyl)amino]-6-({[1-(2- | 0.033 |
| | morpholin-4-ylethyl)-1H-imidazol-4- | |
| | yl]methyl}amino)quinoline-3-carbonitrile | |
| 671 | 8-chloro-4-[(3-chloro-4-fluorophenyl)amino]-6-({[1-(2- | 0.062 |
| | morpholin-4-ylethyl)-1H-imidazol-5- | |
| | yl]methyl}amino)quinoline-3-carbonitrile | |
| 672 | 8-bromo-6-{[(1-tert-butyl-1H-1,2,3-triazol-4- | 0.061 |
| | yl)methyl]amino}-4-[(3-chloro-4- | |
| | fluorophenyl)amino]quinoline-3-carbonitrile | |
| 673 | 8-bromo-4-[(3-chloro-4-fluorophenyl)amino]-6-({[1-(2,2,2- | 0.1 |
| | trifluoroethyl)-1H-1,2,3-triazol-4- | |
| | yl]methyl}amino)quinoline-3-carbonitrile | |
| 674 | tert-butyl 4-(2-{4-[({8-chloro-4-[(3-chloro-4- | 0.075 |
| | fluorophenyl)amino]-3-cyanoquinolin-6-yl}amino)methyl]- | |
| | 1H-1,2,3-triazol-1-yl}ethyl)piperidine-1-carboxylate | |
| 675 | tert-butyl 4-(2-{4-[({8-bromo-4-[(3-chloro-4- | 0.13 |
| | fluorophenyl)amino]-3-cyanoquinolin-6-yl}amino)methyl]- | |
| | 1H-1,2,3-triazol-1-yl}ethyl)piperidine-1-carboxylate | |
| 676 | tert-butyl (2-{4-[({8-chloro-4-[(3-chloro-4- | 0.048 |
| | fluorophenyl)amino]-3-cyanoquinolin-6-yl}amino)methyl]- | |
| | 1H-1,2,3-triazol-1-yl}ethyl)methylcarbamate | |
| 677 | di-tert-butyl [({8-bromo-4-[(3-chloro-4- | 4.7 |
| | fluorophenyl)amino]-3-cyanoquinolin-6- | |
| | yl}imino)bis(methylene-1H-1,2,3-triazole-4,1-diylethane- | |
| | 2,1-diyl)]bis(methylcarbamate) | |
| 678 | tert-butyl (2-{4-[({8-bromo-4-[(3-chloro-4- | 0.067 |
| | fluorophenyl)amino]-3-cyanoquinolin-6-yl}amino)methyl]- | |
| | 1H-1,2,3-triazol-1-yl}ethyl)methylcarbamate | |
| 679 | 8-bromo-4-[(3-chloro-4-fluorophenyl)amino]-6- | 0.29 |
| | [methyl({1-[(1-methylpiperidin-4-yl)methyl]-1H-1,2,3- | |
| | triazol-4-yl}methyl)amino]quinoline-3-carbonitrile | |
| 680 | 8-bromo-4-[(3-chloro-4-fluorophenyl)amino]-6-[({1-[(1- | 0.012 |
| | methylpiperidin-4-yl)methyl]-1H-1,2,3-triazol-4- | |
| | yl}methyl)amino]quinoline-3-carbonitrile | |
| 681 | 8-bromo-4-[(3-chloro-4-fluorophenyl)amino]-6- | 0.022 |
| | (methyl{[1-(1-methylpiperidin-4-yl)-1H-1,2,3-triazol-4- | |
| | yl]methyl}amino)quinoline-3-carbonitrile | |
| 682 | 8-bromo-4-[(3-chloro-4-fluorophenyl)amino]-6-[({1-[2- | 0.082 |
| | (methylthio)ethyl]-1H-1,2,3-triazol-4- | |
| | yl}methyl)amino]quinoline-3-carbonitrile | |
| 683 | 8-bromo-4-[(3-chloro-4-fluorophenyl)amino]-6-({[4-(3- | 0.062 |
| | methoxypropyl)-1H-imidazol-5- | |
| | yl]methyl}amino)quinoline-3-carbonitrile | |
| 684 | 8-bromo-4-[(3-chloro-4-fluorophenyl)amino]-6-[({1-[2- | 0.038 |
| | (methylamino)ethyl]-1H-1,2,3-triazol-4- | |
| | yl}methyl)amino]quinoline-3-carbonitrile | |
| 685 | 8-chloro-4-[(3-chloro-4-fluorophenyl)amino]-6-[({1-[2- | 0.029 |
| | (methylamino)ethyl]-1H-1,2,3-triazol-4- | |
| | yl}methyl)amino]quinoline-3-carbonitrile | |
| 686 | 8-bromo-4-[(3-chloro-4-fluorophenyl)amino]-6-({[1-(2- | 0.028 |
| | hydroxyethyl)-1H-1,2,3-triazol-4- | |
| | yl]methyl}amino)quinoline-3-carbonitrile | |
| 867 | 8-chloro-4-[(3-chloro-4-fluorophenyl)amino]-6-[({1-[3- | 0.002 |
| | (dimethylamino)propyl]-1H-1,2,3-triazol-4- | |
| | yl}methyl)amino]quinoline-3-carbonitrile | |
| 688 | 8-chloro-4-[(3-chloro-4-fluorophenyl)amino]-6-({[1-(2- | 0.04 |
| | pyrrolidin-1-ylethyl)-1H-1,2,3-triazol-4- | |
| | yl]methyl}amino)quinoline-3-carbonitrile | |
| 689 | 8-chloro-4-[(3-chloro-4-fluorophenyl)amino]-6-[({1-[2-(1- | 0.015 |
| | methylpyrrolidin-2-yl)ethyl]-1H-1,2,3-triazol-4- | |
| | yl}methyl)amino]quinoline-3-carbonitrile | |
| 690 | 8-chloro-4-[(3-chloro-4-fluorophenyl)amino]-6-({[1- | 0.01 |
| | (pyridin-3-ylmethyl)-1H-1,2,3-triazol-4- | |
| | yl]methyl}amino)quinoline-3-carbonitrile | |
| 691 | 8-bromo-4-[(3-chloro-4-fluorophenyl)amino]-6-({[1-(2- | 0.09 |
| | piperidin-4-ylethyl)-1H-1,2,3-triazol-4- | |
| | yl]methyl}amino)quinoline-3-carbonitrile | |
| 692 | 8-chloro-4-[(3-chloro-4-fluorophenyl)amino]-6-({[1-(2- | 0.13 |
| | piperidin-4-ylethyl)-1H-1,2,3-triazol-4- | |
| | yl]methyl}amino)quinoline-3-carbonitrile | |
| 693 | di-tert-butyl 4,4′-[({8-bromo-4-[(3-chloro-4- | >3 |
| | fluorophenyl)amino]-3-cyanoquinolin-6- | |
| | yl}imino)bis(methylene-1H-1,2,3-triazole-4,1-diylethane- | |
| | 2,1-diyl)]dipiperidine-1-carboxylate | |
| 694 | 8-bromo-4-[(3-chloro-4-fluorophenyl)amino]-6-[({1-[2- | 0.091 |
| | (methylsulfonyl)ethyl]-1H-1,2,3-triazol-4- | |
| | yl}methyl)amino]quinoline-3-carbonitrile | |
| 695 | 8-bromo-4-[(3-chloro-4-fluorophenyl)amino]-6-[({1-[2-(1- | 0.044 |
| | methylpiperidin-4-yl)ethyl]-1H-1,2,3-triazol-4- | |
| | yl}methyl)amino]quinoline-3-carbonitrile | |
| 696 | 8-chloro-4-[(3-chloro-4-fluorophenyl)amino]-6-[({1-[2-(1- | 0.0017 |
| | methylpiperidin-4-yl)ethyl]-1H-1,2,3-triazol-4- | |
| | yl}methyl)amino]quinoline-3-carbonitrile | |
| 697 | 8-bromo-4-[(3-chloro-4-fluorophenyl)amino]-6-({[1-(3- | 0.018 |
| | hydroxypropyl)-1H-1,2,3-triazol-4- | |
| | yl]methyl}amino)quinoline-3-carbonitrile | |
| 698 | 8-bromo-4-[(3-chloro-4-fluorophenyl)amino]-6-({[1-(2- | 0.018 |
| | methoxyethyl)-1H-1,2,3-triazol-4- | |
| | yl]methyl}amino)quinoline-3-carbonitrile | |
| 699 | 8-bromo-4-[(3-chloro-4-fluorophenyl)amino]-6-[({1-[2- | 0.053 |
| | (cyclopentylsulfonyl)ethyl]-1H-1,2,3-triazol-4- | |
| | yl}methyl)amino]quinoline-3-carbonitrile | |
| 700 | 8-bromo-4-[(3-chloro-4-fluorophenyl)amino]-6- | 0.11 |
| | [(imidazo[1,2-a]pyrazin-3-ylmethyl)amino]quinoline-3- | |
| | carbonitrile | |
| 701 | 8-bromo-4-[(3-chloro-4-fluorophenyl)amino]-6- | 0.28 |
| | [(imidazo[1,2-a]pyridin-3-ylmethyl)amino]quinoline-3- | |
| | carbonitrile | |
| 702 | 8-bromo-4-[(3-chloro-4-fluorophenyl)amino]-6-[({1-[2- | 0.043 |
| | (methylsulfonyl)ethyl]-1H-imidazol-4- | |
| | yl}methyl)amino]quinoline-3-carbonitrile | |
| 703 | 8-bromo-4-[(3-chloro-4-fluorophenyl)amino]-6-[({1- | 0.12 |
| | [(methylsulfinyl)methyl]-1H-1,2,3-triazol-4- | |
| | yl}methyl)amino]quinoline-3-carbonitrile | |
| 704 | 8-bromo-4-[(3-chloro-4-fluorophenyl)amino]-6-[({1- | 0.033 |
| | [(methylsulfonyl)methyl]-1H-1,2,3-triazol-4- | |
| | yl}methyl)amino]quinoline-3-carbonitrile | |
| 705 | 8-bromo-4-[(3-chloro-4-fluorophenyl)amino]-6-[({1- | 0.012 |
| | [(methylthio)methyl]-1H-1,2,3-triazol-4- | |
| | yl}methyl)amino]quinoline-3-carbonitrile | |
| 706 | 8-bromo-4-[(3-chloro-4-fluorophenyl)amino]-6-({[1- | 0.0031 |
| | (pyridin-3-ylmethyl)-1H-imidazol-4- | |
| | yl]methyl}amino)quinoline-3-carbonitrile | |
| 707 | 8-bromo-4-[(3-chloro-4-fluorophenyl)amino]-6-{[(3- | 0.8 |
| | piperidin-4-yl-1H-pyrazol-5-yl)methyl]amino}quinoline-3- | |
| | carbonitrile | |
| 708 | 8-bromo-4-[(3-chloro-4-fluorophenyl)amino]-6-[({1-[2-(1- | 0.034 |
| | methylpyrrolidin-2-yl)ethyl]-1H-imidazol-4- | |
| | yl}methyl)amino]quinoline-3-carbonitrile | |
| 709 | 6-{[(1-allyl-1H-imidazol-4-yl)methyl]amino}-8-bromo-4- | 0.0048 |
| | [(3-chloro-4-fluorophenyl)amino]quinoline-3-carbonitrilef | |
| 710 | 8-bromo-4-[(3-chloro-4-fluorophenyl)amino]-6-[({1-[1- | 0.0011 |
| | (methylsulfonyl)piperidin-4-yl]-1H-1,2,3-triazol-4- | |
| | yl}methyl)amino]quinoline-3-carbonitrile | |
| 711 | 8-bromo-4-[(3-chloro-4-fluorophenyl)amino]-6-({[1-(3- | 0.0034 |
| | piperidin-1-ylpropyl)-1H-1,2,3-triazol-4- | |
| | yl]methyl}amino)quinoline-3-carbonitrile | |
| 712 | 8-chloro-4-[(3-chloro-4-fluorophenyl)amino]-6-({[1-(3- | 0.0011 |
| | piperidin-1-ylpropyl)-1H-1,2,3-triazol-4- | |
| | yl]methyl}amino)quinoline-3-carbonitrile | |
| 713 | tert-butyl 3-({4-[({8-bromo-4-[(3-chloro-4- | 0.12 |
| | fluorophenyl)amino]-3-cyanoquinolin-6-yl}amino)methyl]- | |
| | 1H-1,2,3-triazol-1-yl}methyl)piperidine-1-carboxylate | |
| 714 | tert-butyl 3-({4-[({8-chloro-4-[(3-chloro-4- | 0.25 |
| | fluorophenyl)amino]-3-cyanoquinolin-6-yl}amino)methyl]- | |
| | 1H-1,2,3-triazol-1-yl}methyl)piperidine-1-carboxylate | |
| 715 | 8-bromo-4-[(3-chloro-4-fluorophenyl)amino]-6-[({1-[1- | 0.19 |
| | (2,2-dimethylpropyl)piperidin-4-yl]-1H-1,2,3-triazol-4- | |
| | yl}methyl)amino]quinoline-3-carbonitrile | |
| 716 | tert-butyl 2-({4-[({8-chloro-4-[(3-chloro-4- | 0.32 |
| | fluorophenyl)amino]-3-cyanoquinolin-6-yl}amino)methyl]- | |
| | 1H-1,2,3-triazol-1-yl}methyl)pyrrolidine-1-carboxylate | |
| 717 | tert-butyl 2-({4-[({8-bromo-4-[(3-chloro-4- | 0.27 |
| | fluorophenyl)amino]-3-cyanoquinolin-6-yl}amino)methyl]- | |
| | 1H-1,2,3-triazol-1-yl}methyl)pyrrolidine-1-carboxylate | |
| 718 | 8-bromo-4-[(3-chloro-4-fluorophenyl)amino]-6-({[1- | 0.033 |
| | (piperidin-3-ylmethyl)-1H-1,2,3-triazol-4- | |
| | yl]methyl}amino)quinoline-3-carbonitrile | |
| 719 | 8-chloro-4-[(3-chloro-4-fluorophenyl)amino]-6-({[1- | 0.048 |
| | (piperidin-3-ylmethyl)-1H-1,2,3-triazol-4- | |
| | yl]methyl}amino)quinoline-3-carbonitrile | |
| 720 | 6-({[1-(1-acetylpiperidin-4-yl)-1H-1,2,3-triazol-4- | 0.0061 |
| | yl]methyl}amino)-8-bromo-4-[(3-chloro-4- | |
| | fluorophenyl)amino]quinoline-3-carbonitrile | |
| 721 | 8-chloro-4-[(3-chloro-4-fluorophenyl)amino]-6-({[1- | 0.0091 |
| | (pyrrolidin-2-ylmethyl)-1H-1,2,3-triazol-4- | |
| | yl]methyl}amino)quinoline-3-carbonitrile | |
| 722 | 8-bromo-4-[(3-chloro-4-fluorophenyl)amino]-6-({[1- | 0.0092 |
| | (pyrrolidin-2-ylmethyl)-1H-1,2,3-triazol-4- | |
| | yl]methyl}amino)quinoline-3-carbonitrile | |
| 723 | 6-({[1-(2-azepan-1-ylethyl)-1H-1,2,3-triazol-4- | 0.0028 |
| | yl]methyl}amino)-8-chloro-4-[(3-chloro-4- | |
| | fluorophenyl)amino]quinoline-3-carbonitrile | |
| 724 | 8-bromo-4-[(3-chloro-4-fluorophenyl)amino]-6-[({3-[(2S)- | 0.33 |
| | pyrrolidin-2-yl]-1H-pyrazol-5-yl}methyl)amino]quinoline- | |
| | 3-carbonitrile | |
| 725 | 8-bromo-4-[(3-chloro-4-fluorophenyl)amino]-6-{[(1- | 0.023 |
| | pyridin-4-yl-1H-imidazol-4-yl)methyl]amino}quinoline-3- | |
| | carbonitrile | |
| 726 | 8-chloro-4-[(3-chloro-4-fluorophenyl)amino]-6-({[1- | 0.007 |
| | (pyridin-3-ylmethyl)-1H-imidazol-4- | |
| | yl]methyl}amino)quinoline-3-carbonitrile | |
| 727 | 8-chloro-4-[(3-chloro-4-fluorophenyl)amino]-6-({[1- | 0.0041 |
| | (pyridin-4-ylmethyl)-1H-1,2,3-triazol-4- | |
| | yl]methyl}amino)quinoline-3-carbonitrile | |
| 728 | 8-bromo-4-[(3-chloro-4-fluorophenyl)amino]-6-({[1-(2- | 0.0034 |
| | pyridin-2-ylethyl)-1H-1,2,3-triazol-4- | |
| | yl]methyl}amino)quinoline-3-carbonitrile | |
| 729 | 8-bromo-4-[(3-chloro-4-fluorophenyl)amino]-6-{[(1- | 0.0055 |
| | pyridin-4-yl-1H-1,2,3-triazol-4-yl)methyl]amino}quinoline- | |
| | 3-carbonitrile | |
| 730 | 8-chloro-4-[(3-chloro-4-fluorophenyl)amino]-6-{[(1- | 0.0032 |
| | pyridin-4-yl-1H-1,2,3-triazol-4-yl)methyl]amino}quinoline- | |
| | 3-carbonitrile | |
| 731 | 8-chloro-4-[(3-chloro-4-fluorophenyl)amino]-6-[({1-[2- | 0.013 |
| | (dimethylamino)ethyl]-1H-1,2,3-triazol-4- | |
| | yl}methyl)amino]quinoline-3-carbonitrile | |
| 732 | 6-{[(1-allyl-1H-1,2,3-triazol-4-yl)methyl]amino}-8-bromo- | 0.02 |
| | 4-[(3-chloro-4-fluorophenyl)amino]quinoline-3- | |
| | carbonitrile | |
| 733 | 8-chloro-4-[(3-chloro-4-fluorophenyl)amino]-6-({[1-(2- | 0.02 |
| | methoxyethyl)-1H-1,2,3-triazol-4- | |
| | yl]methyl}amino)quinoline-3-carbonitrile | |
| 734 | 8-chloro-4-[(3-chloro-4-fluorophenyl)amino]-6-({[1- | 0.014 |
| | (tetrahydro-2H-pyran-4-yl)-1H-1,2,3-triazol-4- | |
| | yl]methyl}amino)quinoline-3-carbonitrile | |
| 735 | 8-bromo-4-[(3-chloro-4-fluorophenyl)amino]-6-[({1-[(1- | 0.04 |
| | ethylpiperidin-4-yl)methyl]-1H-1,2,3-triazol-4- | |
| | yl}methyl)amino]quinoline-3-carbonitrile | |
| 736 | 8-bromo-4-[(3-chloro-4-fluorophenyl)amino]-6-[({1-[(1- | 0.029 |
| | isopropylpiperidin-4-yl)methyl]-1H-1,2,3-triazol-4- | |
| | yl}methyl)amino]quinoline-3-carbonitrile | |
| 737 | 8-bromo-4-[(3-chloro-4-fluorophenyl)amino]-6-[({1-[2-(1- | 0.064 |
| | ethylpiperidin-4-yl)ethyl]-1H-1,2,3-triazol-4- | |
| | yl}methyl)amino]quinoline-3-carbonitrile | |
| 738 | 8-bromo-4-[(3-chloro-4-fluorophenyl)amino]-6-[({1-[2-(1- | 0.057 |
| | isopropylpiperidin-4-yl)ethyl]-1H-1,2,3-triazol-4- | |
| | yl}methyl)amino]quinoline-3-carbonitrile | |
| 739 | 8-bromo-4-[(3-chloro-4-fluorophenyl)amino]-6-[({1-[2- | 0.093 |
| | (dimethylamino)-1-phenylethyl]-1H-1,2,3-triazol-4- | |
| | yl}methyl)amino]quinoline-3-carbonitrile | |
| 740 | 8-chloro-4-[(3-chloro-4-fluorophenyl)amino]-6-[({1-[2- | 0.07 |
| | (dimethylamino)-1-phenylethyl]-1H-1,2,3-triazol-4- | |
| | yl}methyl)amino]quinoline-3-carbonitrile | |
| 741 | 8-bromo-4-[(3-chloro-4-fluorophenyl)amino]-6-{[(1-{2-[2- | 0.14 |
| | (2-hydroxyethoxy)ethoxy]ethyl}-1H-1,2,3-triazol-4- | |
| | yl)methyl]amino}quinoline-3-carbonitrile | |
| 742 | 8-chloro-4-[(3-chloro-4-fluorophenyl)amino]-6-{[(1-{2-[2- | 0.091 |
| | (2-hydroxyethoxy)ethoxy]ethyl}-1H-1,2,3-triazol-4- | |
| | yl)methyl]amino}quinoline-3-carbonitrile | |
| 743 | 8-bromo-4-[(3-chloro-4-fluorophenyl)amino]-6-[({1-[2-(2- | 0.1 |
| | methoxyethoxy)ethyl]-1H-1,2,3-triazol-4- | |
| | yl}methyl)amino]quinoline-3-carbonitrile | |
| 744 | 8-chloro-4-[(3-chloro-4-fluorophenyl)amino]-6-[({1-[2-(2- | 0.021 |
| | methoxyethoxy)ethyl]-1H-1,2,3-triazol-4- | |
| | yl}methyl)amino]quinoline-3-carbonitrile | |
| 745 | 8-bromo-4-[(3-chloro-4-fluorophenyl)amino]-6-[({4-[2- | 0.24 |
| | (dimethylamino)ethyl]-1H-imidazol-5- | |
| | yl}methyl)amino]quinoline-3-carbonitrile | |
| 746 | 8-chloro-4-[(3-chloro-4-fluorophenyl)amino]-6-({[1-(2- | 0.025 |
| | pyridin-2-ylethyl)-1H-1,2,3-triazol-4- | |
| | yl]methyl}amino)quinoline-3-carbonitrile | |
| 747 | 8-chloro-4-[(3-chloro-4-fluorophenyl)amino]-6-({[1-(2- | 0.32 |
| | piperidin-1-ylpyrimidin-4-yl)-1H-1,2,3-triazol-4- | |
| | yl]methyl}amino)quinoline-3-carbonitrile | |
| 748 | 8-bromo-4-[(3-chloro-4-fluorophenyl)amino]-6-({[1-(1- | 0.015 |
| | oxidopyridin-4-yl)-1H-1,2,3-triazol-4- | |
| | yl]methyl}amino)quinoline-3-carbonitrile | |
| 749 | 8-chloro-4-[(3-chloro-4-fluorophenyl)amino]-6-({[1-(1- | 0.016 |
| | oxidopyridin-4-yl)-1H-1,2,3-triazol-4- | |
| | yl]methyl}amino)quinoline-3-carbonitrile | |
| 750 | 3-{4-[({8-chloro-4-[(3-chloro-4-fluorophenyl)amino]-3- | 0.016 |
| | cyanoquinolin-6-yl}amino)methyl]-1H-1,2,3-triazol-1- | |
| | yl}propyl dimethylcarbamate | |
| 751 | 8-chloro-4-[(3-chloro-4-fluorophenyl)amino]-6-({[1-(3- | 0.0036 |
| | hydroxypropyl)-1H-1,2,3-triazol-4- | |
| | yl]methyl}amino)quinoline-3-carbonitrile | |
| 752 | 3-{4-[({8-bromo-4-[(3-chloro-4-fluorophenyl)amino]-3- | 0.0035 |
| | cyanoquinolin-6-yl}amino)methyl]-1H-1,2,3-triazol-1- | |
| | yl}propyl dimethylcarbamate | |
| 753 | 8-bromo-4-[(3-chloro-4-fluorophenyl)amino]-6-({[1- | 0.033 |
| | (pyridin-2-ylmethyl)-1H-1,2,3-triazol-4- | |
| | yl]methyl}amino)quinoline-3-carbonitrile | |
| 754 | 8-chloro-4-[(3-chloro-4-fluorophenyl)amino]-6-({[1- | 0.0021 |
| | (pyridin-2-ylmethyl)-1H-1,2,3-triazol-4- | |
| | yl]methyl}amino)quinoline-3-carbonitrile | |
| 755 | 8-chloro-4-[(3-chloro-4-fluorophenyl)amino]-6-({[1- | 0.79 |
| | (cyclohexylmethyl)-1H-1,2,3-triazol-4- | |
| | yl]methyl}amino)quinoline-3-carbonitrile | |
| 756 | 8-chloro-4-[(3-chloro-4-fluorophenyl)amino]-6-[({1-[4- | >3 |
| | (trifluoromethyl)benzyl]-1H-1,2,3-triazol-4- | |
| | yl}methyl)amino]quinoline-3-carbonitrile | |
| 757 | 8-bromo-4-[(3-chloro-4-fluorophenyl)amino]-6-[({1-[3- | 0.0067 |
| | (dimethylamino)-1-pyridin-3-ylpropyl]-1H-1,2,3-triazol-4- | |
| | yl}methyl)amino]quinoline-3-carbonitrile | |
| 758 | 8-chloro-4-[(3-chloro-4-fluorophenyl)amino]-6-{[(1- | 0.0076 |
| | pyridin-3-yl-1H-1,2,3-triazol-4-yl)methyl]amino}quinoline- | |
| | 3-carbonitrile | |
| 759 | 8-chloro-4-[(3-chloro-4-fluorophenyl)amino]-6-[({1-[3- | 0.036 |
| | (dimethylamino)-1-pyridin-3-ylpropyl]-1H-1,2,3-triazol-4- | |
| | yl}methyl)amino]quinoline-3-carbonitrile | |
| 760 | methyl 8-chloro-4-[(3-chloro-4-fluorophenyl)amino]-3- | >3 |
| | cyanoquinoline-6-carboxylate | |
| 761 | 8-chloro-4-[(3-chloro-4-fluorophenyl)amino]-6-({[1-(1- | 0.096 |
| | phenylethyl)-1H-1,2,3-triazol-4- | |
| | yl]methyl}amino)quinoline-3-carbonitrile | |
| 762 | tert-butyl 4-(3-{4-[({8-bromo-4-[(3-chloro-4- | 0.52 |
| | fluorophenyl)amino]-3-cyanoquinolin-6-yl}amino)methyl}- | |
| | 1H-1,2,3-triazol-1-yl}propyl)-1,4-diazepane-1- | |
| | carboxylate | |
| 763 | tert-butyl 4-(3-{4-[({8-chloro-4-[(3-chloro-4- | 0.12 |
| | fluorophenyl)amino]-3-cyanoquinolin-6-yl}amino)methyl]- | |
| | 1H-1,2,3-triazol-1-yl}propyl)-1,4-diazepane-1- | |
| | carboxylate | |
| 764 | tert-butyl 4-(3-{4-[({8-bromo-4-[(3-chloro-4- | 1.3 |
| | fluorophenyl)amino]-3-cyanoquinolin-6-yl}amino)methyl]- | |
| | 1H-1,2,3-triazol-1-yl}propyl)piperazine-1-carboxylate | |
| 765 | tert-butyl 4-(3-{4-[({8-chloro-4-[(3-chloro-4- | 0.23 |
| | fluorophenyl)amino]-3-cyanoquinolin-6-yl}amino)methyl]- | |
| | 1H-1,2,3-triazol-1-yl}propyl)piperazine-1-carboxylate | |
| 766 | 8-bromo-4-[(3-chloro-4-fluorophenyl)amino]-6-[({1-[3- | 0.043 |
| | (1,4-diazepan-1-yl)propyl]-1H-1,2,3-triazol-4- | |
| | yl}methyl)amino]quinoline-3-carbonitrile | |
| 767 | 8-chloro-4-[(3-chloro-4-fluorophenyl)amino]-6-[({1-[3- | 0.074 |
| | (1,4-diazepan-1-yl)propyl]-1H-1,2,3-triazol-4- | |
| | yl}methyl)amino]quinoline-3-carbonitrile | |
| 768 | 8-bromo-4-[(3-chloro-4-fluorophenyl)amino]-6-({[1-(3- | 0.11 |
| | piperazin-1-ylpropyl)-1H-1,2,3-triazol-4- | |
| | yl]methyl}amino)quinoline-3-carbonitrile | |
| 769 | 8-chloro-4-[(3-chloro-4-fluorophenyl)amino]-6-({[1-(3- | 0.16 |
| | piperazin-1-ylpropyl)-1H-1,2,3-triazol-4- | |
| | yl]methyl}amino)quinoline-3-carbonitrile | |
| 770 | 8-chloro-4-[(3-chloro-4-fluorophenyl)amino]-3- | 2.4 |
| | cyanoquinoline-6-carboxylic acid | |
| 771 | N2-{8-chloro-4-[(3-chloro-4-fluorophenyl)amino]-3- | 0.2 |
| | cyanoquinolin-6-yl}-N-(pyridin-3-ylmethyl)glycinamide | |
| 772 | N2-{8-chloro-4-[(3-chloro-4-fluorophenyl)amino]-3- | 0.048 |
| | cyanoquinolin-6-yl}-N-(pyridin-4-ylmethyl)glycinamide | |
| 773 | N2-{8-chloro-4-[(3-chloro-4-fluorophenyl)amino]-3- | 0.11 |
| | cyanoquinolin-6-yl}-N-pyridin-3-ylglycinamide | |
| 774 | N2-{8-chloro-4-[(3-chloro-4-fluorophenyl)amino]-3- | 2.4 |
| | cyanoquinolin-6-yl}-N-pyridin-4-ylglycinamide | |
| 775 | N2-{8-chloro-4-[(3-chloro-4-fluorophenyl)amino]-3- | >3 |
| | cyanoquinolin-6-yl}-N-[3- | |
| | (dimethylamino)propyl]glycinamide | |
| 776 | N2-{8-chloro-4-[(3-chloro-4-fluorophenyl)amino]-3- | >3 |
| | cyanoquinolin-6-yl}-N-(3-morpholin-4- | |
| | ylpropyl)glycinamide | |
| 777 | N2-{8-chloro-4-[(3-chloro-4-fluorophenyl)amino]-3- | 0.53 |
| | cyanoquinolin-6-yl}-N-(2-morpholin-4-ylethyl)glycinamide | |
| 778 | N2-{8-chloro-4-[(3-chloro-4-fluorophenyl)amino]-3- | 1.6 |
| | cyanoquinolin-6-yl}-N-[2- | |
| | (dimethylamino)ethyl]glycinamide | |
| 779 | N2-{8-chloro-4-[(3-chloro-4-fluorophenyl)amino]-3- | 0.23 |
| | cyanoquinolin-6-yl}-N-(3-methoxypropyl)glycinamide | |
| 780 | N2-{8-chloro-4-[(3-chloro-4-fluorophenyl)amino]-3- | 0.33 |
| | cyanoquinolin-6-yl}-N-(pyridin-2-ylmethyl)glycinamide | |
| 781 | N2-{8-chloro-4-[(3-chloro-4-fluorophenyl)amino]-3- | >3 |
| | cyanoquinolin-6-yl}-N-[2-(1-methylpyrrolidin-2- | |
| | yl)ethyl]glycinamide | |
| 782 | 8-chloro-6-[(2-chlorocyclopentyl)amino]-4-[(3-chloro-4- | >3 |
| | fluorophenyl)amino]quinoline-3-carbonitrile | |
| 783 | 8-chloro-4-[(3-chloro-4-fluorophenyl)amino]-6-{[2-(2l5- | 0.42 |
| | triaz-1-en-2-yn-1-yl)cyclopentyl]amino}quinoline-3- | |
| | carbonitrile | |
| 784 | 2-{4-[({8-chloro-4-[(3-chloro-4-fluorophenyl)amino]-3- | 0.0059 |
| | cyanoquinolin-6-yl}amino)methyl]-1H-1,2,3-triazol-1- | |
| | yl}ethyl carbamate | |
| 785 | 8-bromo-4-[(3-chloro-4-fluorophenyl)amino]-6-({[1- | 0.034 |
| | (pyridin-2-ylmethyl)-1H-imidazol-4- | |
| | yl]methyl}amino)quinoline-3-carbonitrile | |
| 786 | 8-chloro-4-[(3-chloro-4-fluorophenyl)amino]-6-({[1- | 0.041 |
| | (pyridin-2-ylmethyl)-1H-imidazol-4- | |
| | yl]methyl}amino)quinoline-3-carbonitrile | |
| 787 | 8-chloro-4-[(3-chloro-4-fluorophenyl)amino]-6-({[1- | 0.0053 |
| | (pyridin-4-ylmethyl)-1H-imidazol-4- | |
| | yl]methyl}amino)quinoline-3-carbonitrile | |
| 788 | 2-{4-[({8-chloro-4-[(3-chloro-4-fluorophenyl)amino]-3- | 0.005 |
| | cyanoquinolin-6-yl}amino)methyl]-1H-1,2,3-triazol-1-yl}- | |
| | N,N-dimethylacetamide | |
| 789 | 3-{4-[({8-chloro-4-[(3-chloro-4-fluorophenyl)amino]-3- | 0.01 |
| | cyanoquinolin-6-yl}amino)methyl]-1H-1,2,3-triazol-1-yl}- | |
| | N,N-dimethylpropanamide | |
| 790 | 8-chloro-4-[(3-chloro-4-fluorophenyl)amino]-6-[({1-[2- | 0.0061 |
| | (1,3-dimethyl-2-oxoimidazolidin-4-yl)ethyl]-1H-1,2,3- | |
| | triazol-4-yl}methyl)amino]quinoline-3-carbonitrile | |
| 791 | 8-chloro-4-[(3-chloro-4-fluorophenyl)amino]-6-[({1-[2-(1- | 0.025 |
| | ethyl-2-oxo-3-phenylimidazolidin-4-yl)ethyl]-1H-1,2,3- | |
| | triazol-4-yl}methyl)amino]quinoline-3-carbonitrile | |
| 792 | 8-chloro-4-[(3-chloro-4-fluorophenyl)amino]-6-[({1-[2-(1- | 0.13 |
| | ethyl-3-phenyl-2-thioxoimidazolidin-4-yl)ethyl]-1H-1,2,3- | |
| | triazol-4-yl}methyl)amino]quinoline-3-carbonitrile | |
| 793 | 8-bromo-4-[(3-chloro-4-fluorophenyl)amino]-6-[({1-[3-(4- | 0.07 |
| | methyl-1,4-diazepan-1-yl)propyl]-1H-1,2,3-triazol-4- | |
| | yl}methyl)amino]quinoline-3-carbonitrile | |
| 794 | 8-chloro-4-[(3-chloro-4-fluorophenyl)amino]-6-[({1-[3-(4- | 0.048 |
| | methyl-1,4-diazepan-1-yl)propyl]-1H-1,2,3-triazol-4- | |
| | yl}methyl)amino]quinoline-3-carbonitrile | |
| 795 | 8-bromo-4-[(3-chloro-4-fluorophenyl)amino]-6-[({1-[3-(4- | 0.029 |
| | methylpiperazin-1-yl)propyl]-1H-1,2,3-triazol-4- | |
| | yl}methyl)amino]quinoline-3-carbonitrile | |
| 796 | 8-chloro-4-[(3-chloro-4-fluorophenyl)amino]-6-[({1-[3-(4- | 0.039 |
| | methylpiperazin-1-yl)propyl]-1H-1,2,3-triazol-4- | |
| | yl}methyl)amino]quinoline-3-carbonitrile | |
| 797 | 8-chloro-4-[(3-chloro-4-fluorophenyl)amino]-6-[(2,3- | 0.59 |
| | dihydroxypropyl)amino]quinoline-3-carbonitrile | |
| 798 | N-{8-bromo-4-[(3-chloro-4-fluorophenyl)amino]-3- | 0.14 |
| | cyanoquinolin-6-yl}glycine | |
| 799 | 8-bromo-4-[(3-chloro-4-fluorophenyl)amino]-6-{[(1- | 0.23 |
| | cyclopentyl-1H-imidazol-4-yl)methyl]amino}quinoline-3- | |
| | carbonitrile | |
| 800 | 8-chloro-4-[(3-chloro-4-fluorophenyl)amino]-6-[({1-[3- | 0.15 |
| | (3,5-dimethylpiperidin-1-yl)propyl]-1H-1,2,3-triazol-4- | |
| | yl}methyl)amino]quinoline-3-carbonitrile | |
| 801 | 8-chloro-4-[(3-chloro-4-fluorophenyl)amino]-6-{[(1-{3- | 0.0018 |
| | [cyclohexyl(methyl)amino]propyl}-1H-1,2,3-triazol-4- | |
| | yl)methyl]amino}quinoline-3-carbonitrile | |
| 802 | N-(4-{4-[({8-chloro-4-[(3-chloro-4-fluorophenyl)amino]-3- | 0.035 |
| | cyanoquinolin-6-yl}amino)methyl]-1H-1,2,3-triazol-1- | |
| | yl}phenyl)acetamide | |
| 803 | 8-chloro-4-[(3-chloro-4-fluorophenyl)amino]-6-({2-[4-(2- | 0.067 |
| | hydroxyethyl)-1H-1,2,3-triazol-1- | |
| | yl]cyclopentyl}amino)quinoline-3-carbonitrile | |
| 804 | 8-bromo-4-[(3-chloro-4-fluorophenyl)amino]-6-{[(1- | 0.068 |
| | isopropyl-1H-imidazol-4-yl)methyl]amino}quinoline-3- | |
| | carbonitrile | |
| 805 | 6-[({1-[(benzyloxy)methyl]-1H-1,2,3-triazol-4- | >3 |
| | yl}methyl)amino]-8-bromo-4-[(3-chloro-4- | |
| | fluorophenyl)amino]quinoline-3-carbonitrile | |
| 806 | 8-chloro-4-[(3-chloro-4-fluorophenyl)amino]-6-[(2-pyridin- | 0.2 |
| | 4-ylpropyl)amino]quinoline-3-carbonitrile | |
| 807 | 6-[({1-[(benzyloxy)methyl]-1H-1,2,3-triazol-4- | >3 |
| | yl}methyl)amino]-8-chloro-4-[(3-chloro-4- | |
| | fluorophenyl)amino]quinoline-3-carbonitrile | |
| 808 | {4-[({8-chloro-4-[(3-chloro-4-fluorophenyl)amino]-3- | 0.0096 |
| | cyanoquinolin-6-yl}amino)methyl]-1H-1,2,3-triazol-1- | |
| | yl}acetic acid | |
| 809 | 8-chloro-4-[(3-chloro-4-fluorophenyl)amino]-6-({[(2S)-1- | 0.33 |
| | ethylpyrrolidin-2-yl]methyl}amino)quinoline-3-carbonitrile | |
| 810 | 6-({[(2S)-1-acetylpyrrolidin-2-yl]methyl}amino)-8-chloro- | 0.054 |
| | 4-[(3-chloro-4-fluorophenyl)amino]quinoline-3- | |
| | carbonitrile | |
| 811 | 8-chloro-4-[(3-chloro-4-fluorophenyl)amino]-6- | 1.2 |
| | (ethyl{[(2S)-1-ethylpyrrolidin-2- | |
| | yl]methyl}amino)quinoline-3-carbonitrile | |
| 812 | 8-chloro-4-[(3-chloro-4-fluorophenyl)amino]-6-({[(4S)- | 0.2 |
| | 2,2-dimethyl-1,3-dioxolan-4-yl]methyl}amino)quinoline-3- | |
| | carbonitrile | |
| 813 | 2-{4-[({8-chloro-4-[(3-chloro-4-fluorophenyl)amino]-3- | 0.0035 |
| | cyanoquinolin-6-yl}amino)methyl]-1H-1,2,3-triazol-1-yl}- | |
| | N-isopropylacetamide | |
| 814 | 6-({[1-(2-azepan-1-yl-2-oxoethyl)-1H-1,2,3-triazol-4- | 0.0046 |
| | yl]methyl}amino)-8-chloro-4-[(3-chloro-4- | |
| | fluorophenyl)amino]quinoline-3-carbonitrile | |
| 815 | 6-({[1-(2-azocan-1-yl-2-oxoethyl)-1H-1,2,3-triazol-4- | 0.0053 |
| | yl]methyl}amino)-8-chloro-4-[(3-chloro-4- | |
| | fluorophenyl)amino]quinoline-3-carbonitrile | |
| 816 | 2-{4-[({8-chloro-4-[(3-chloro-4-fluorophenyl)amino]-3- | 0.015 |
| | cyanoquinolin-6-yl}amino)methyl]-1H-1,2,3-triazol-1-yl}- | |
| | N-(2-methoxyethyl)acetamide | |
| 817 | 2-{4-[({8-chloro-4-[(3-chloro-4-fluorophenyl)amino]-3- | 0.0069 |
| | cyanoquinolin-6-yl}amino)methyl]-1H-1,2,3-triazol-1-yl}- | |
| | N-[2-(1-methylpyrrolidin-2-yl)ethyl]acetamide | |
| 818 | 2-{4-[({8-chloro-4-[(3-chloro-4-fluorophenyl)amino]-3- | 0.0068 |
| | cyanoquinolin-6-yl}amino)methyl]-1H-1,2,3-triazol-1-yl}- | |
| | N-[2-(dimethylamino)ethyl]acetamide | |
| 819 | 2-{4-[({8-chloro-4-[(3-chloro-4-fluorophenyl)amino]-3- | 0.0008 |
| | cyanoquinolin-6-yl}amino)methyl]-1H-1,2,3-triazol-1-yl}- | |
| | N-(pyridin-3-ylmethyl)acetamide | |
| 820 | 2-{4-[({8-chloro-4-[(3-chloro-4-fluorophenyl)amino]-3- | 0.014 |
| | cyanoquinolin-6-yl}amino)methyl]-1H-1,2,3-triazol-1-yl}- | |
| | N-(2-morpholin-4-ylethyl)acetamide | |
| 821 | 2-{4-[({8-chloro-4-[(3-chloro-4-fluorophenyl)amino]-3- | 0.0027 |
| | cyanoquinolin-6-yl}amino)methyl]-1H-1,2,3-triazol-1-yl}- | |
| | N-pyridin-3-ylacetamide | |
| 822 | 8-chloro-4-[(3-chloro-4-fluorophenyl)amino]-6-[({1-[2-(4- | 0.013 |
| | methylpiperazin-1-yl)-2-oxoethyl]-1H-1,2,3-triazol-4- | |
| | yl}methyl)amino]quinoline-3-carbonitrile | |
| 823 | 6-[bis({1-[2-(1-methylpyrrolidin-2-yl)ethyl]-1H-1,2,3- | 0.86 |
| | triazol-4-yl}methyl)amino]-8-bromo-4-[(3-chloro-4- | |
| | fluorophenyl)amino]quinoline-3-carbonitrile | |
| 824 | 8-chloro-4-[(3-chloro-4-fluorophenyl)amino]-6-[(piperidin- | 1.24 |
| | 4-ylmethyl)amino]quinoline-3-carbonitrile | |
| 825 | 8-chloro-4-[(3-chloro-4-fluorophenyl)amino]-6-[({1-[(1- | 0.037 |
| | methylpiperidin-4-yl)methyl]-1H-1,2,3-triazol-4- | |
| | yl}methyl)amino]quinoline-3-carbonitrile | |
| 826 | 8-chloro-4-[(3-chloro-4-fluorophenyl)amino]-6-[({1-[(1- | 0.035 |
| | ethylpiperidin-4-yl)methyl]-1H-1,2,3-triazol-4- | |
| | yl}methyl)amino]quinoline-3-carbonitrile | |
| 827 | 8-chloro-4-[(3-chloro-4-fluorophenyl)amino]-6-[({1-[(1- | 0.011 |
| | isopropylpiperidin-4-yl)methyl]-1H-1,2,3-triazol-4- | |
| | yl}methyl)amino]quinoline-3-carbonitrile | |
| 828 | 4-({4-[({8-chloro-4-[(3-chloro-4-fluorophenyl)amino]-3- | 0.0026 |
| | cyanoquinolin-6-yl}amino)methyl]-1H-1,2,3-triazol-1- | |
| | yl}methyl)-N,N-dimethylpiperidine-1-carboxamide | |
| 829 | 8-chloro-4-[(3-chloro-4-fluorophenyl)amino]-6-[({1-[2-(1- | 0.0018 |
| | ethylpiperidin-4-yl)ethyl]-1H-1,2,3-triazol-4- | |
| | yl}methyl)amino]quinoline-3-carbonitrile | |
| 830 | 4-(2-{4-[({8-chloro-4-[(3-chloro-4-fluorophenyl)amino]-3- | 0.019 |
| | cyanoquinolin-6-yl}amino)methyl]-1H-1,2,3-triazol-1- | |
| | yl}ethyl)-N,N-dimethylpiperidine-1-carboxamide | |
| 831 | 4-(2-{4-[({8-chloro-4-[(3-chloro-4-fluorophenyl)amino]-3- | 0.0093 |
| | cyanoquinolin-6-yl}amino)methyl]-1H-1,2,3-triazol-1- | |
| | yl}ethyl)-N-ethylpiperidine-1-carboxamide | |
| 832 | 6-[({1-[2-(1-acetylpiperidin-4-yl)ethyl]-1H-1,2,3-triazol-4- | 0.0012 |
| | yl}methyl)amino]-8-chloro-4-[(3-chloro-4- | |
| | fluorophenyl)amino]quinoline-3-carbonitrile | |
| 833 | 2-{4-[({8-bromo-4-[(3-chloro-4-fluorophenyl)amino]-3- | 0.009 |
| | cyanoquinolin-6-yl}amino)methyl]-1H-1,2,3-triazol-1- | |
| | yl}ethyl carbamate | |
| 834 | 2-{4-[({8-bromo-4-[(3-chloro-4-fluorophenyl)amino]-3- | 0.014 |
| | cyanoquinolin-6-yl}amino)methyl]-1H-1,2,3-triazol-1-yl}- | |
| | N,N-dimethylacetamide | |
| 835 | 2-(4-{[{8-bromo-4-[(3-chloro-4-fluorophenyl)amino]-3- | 0.0091 |
| | cyanoquinolin-6-yl}(ethyl)amino]methyl}-1H-1,2,3-triazol- | |
| | 1-yl)-N,N-dimethylacetamide | |
| 836 | 2-(4-{[{8-bromo-4-[(3-chloro-4-fluorophenyl)amino]-3- | 0.19 |
| | cyanoquinolin-6-yl}(ethyl)amino]methyl}-1H-1,2,3-triazol- | |
| | 1-yl)-N,N-dimethylacetamide | |
| 837 | 8-chloro-4-[(3-chloro-4-fluorophenyl)amino]-6-[({1-[(2- | 0.011 |
| | methylpyridin-4-yl)methyl]-1H-1,2,3-triazol-4- | |
| | yl}methyl)amino]quinoline-3-carbonitrile | |
| 838 | 6-({[1-(1-azabicyclo[2.2.2]oct-3-yl)-1H-1,2,3-triazol-4- | 0.01 |
| | yl]methyl}amino)-8-bromo-4-[(3-chloro-4- | |
| | fluorophenyl)amino]quinoline-3-carbonitrile | |
| 839 | 6-({[1-(1-azabicylo[2.2.2]oct-3-yl)-1H-1,2,3-triazol-4- | 0.0018 |
| | yl]methyl}amino)-8-chloro-4-[(3-chloro-4- | |
| | fluorophenyl)amino]quinoline-3-carbonitrile | |
| 840 | 8-chloro-4-[(3-chloro-4-fluorophenyl)amino]-6-[({1-[(2- | 0.01 |
| | methylpyridin-3-yl)methyl]-1H-1,2,3-triazol-4- | |
| | yl}methyl)amino]quinoline-3-carbonitrile | |
| 841 | 8-chloro-4-[(3-chloro-4-fluorophenyl)amino]-6-[({1-[3- | 0.029 |
| | (4,4-difluoropiperidin-1-yl)propyl]-1H-1,2,3-triazol-4- | |
| | yl}methyl)amino]quinoline-3-carbonitrile | |
| 842 | 8-chloro-4-[(3-chloro-4-fluorophenyl)amino]-6-[({1-[(2- | 0.0009 |
| | methyl-1-oxidopyridin-3-yl)methyl]-1H-1,2,3-triazol-4- | |
| | yl}methyl)amino]quinoline-3-carbonitrile | |
| 843 | N-[2-({8-chloro-4-[(3-chloro-4-fluorophenyl)amino]-3- | 0.058 |
| | cyanoquinolin-6-yl}amino)ethyl]-N-(pyridin-3- | |
| | ylmethyl)acetamide | |
| 844 | N-[2-({8-chloro-4-[(3-chloro-4-fluorophenyl)amino]-3- | 0.06 |
| | cyanoquinolin-6-yl}amino)ethyl]-N-(pyridin-4- | |
| | ylmethyl)acetamide | |
| 845 | 8-chloro-4-[(3-chloro-4-fluorophenyl)amino]-6-[(pyridin-4- | 0.022 |
| | ylmethyl)amino]quinoline-3-carbonitrile | |
| 846 | N-[2-({8-chloro-4-[(3-chloro-4-fluorophenyl)amino]-3- | 0.17 |
| | cyanoquinolin-6-yl}amino)ethyl]-N-(pyridin-2- | |
| | ylmethyl)acetamide | |
| 847 | 8-chloro-4-[(3-chloro-4-fluorophenyl)amino]-6-{[(1- | 0.17 |
| | isobutyl-1H-1,2,3-triazol-4-yl)methyl]amino}quinoline-3- | |
| | carbonitrile | |
| 848 | 8-chloro-4-[(3-chloro-4-fluorophenyl)amino]-6-(ethyl{[1- | 0.38 |
| | (pyridin-3-ylcarbonyl)piperidin-4- | |
| | yl]methyl}amino)quinoline-3-carbonitrile | |
| 849 | 8-chloro-4-[(3-chloro-4-fluorophenyl)amino]-6-{[(1- | 1.91 |
| | methylpiperidin-4-yl)methyl]amino}quinoline-3- | |
| | carbonitrile | |
| 850 | 8-chloro-4-[(3-chloro-4-fluorophenyl)amino]-6-({[1- | 0.13 |
| | (pyridin-3-ylcarbonyl)piperidin-4- | |
| | yl]methyl}amino)quinoline-3-carbonitrile | |
| 851 | 2-(4-{[{8-chloro-4-[(3-chloro-4-fluorophenyl)amino]-3- | 0.15 |
| | cyanoquinolin-6-yl}(methyl)amino]methyl}-1H-1,2,3- | |
| | triazol-1-yl)-N,N-dimethylacetamide | |
| 852 | 8-chloro-4-[(3-chloro-4-fluorophenyl)amino]-6-[({1-[3- | 0.12 |
| | (1,3-dioxo-1,3-dihydro-2H-isoindol-2-yl)propyl]-1H-1,2,3- | |
| | triazol-4-yl}methyl)amino]quinoline-3-carbonitrile | |
| 853 | N-[2-({8-chloro-4-[(3-chloro-4-fluorophenyl)amino]-3- | 0.88 |
| | cyanoquinolin-6-yl}amino)ethyl]methanesulfonamide | |
| 854 | N-[2-({8-chloro-4-[(3-chloro-4-fluorophenyl)amino]-3- | 1.96 |
| | cyanoquinolin-6-yl}amino)ethyl]benzenesulfonamide | |
| 855 | N-[2-({8-chloro-4-[(3-chloro-4-fluorophenyl)amino]-3- | >3 |
| | cyanoquinolin-6-yl}amino)ethyl]pyridine-3-sulfonamide | |
| 856 | N-[2-({8-chloro-4-[(3-chloro-4-fluorophenyl)amino]-3- | 0.82 |
| | cyanoquinolin-6-yl}amino)ethyl]pyridine-2-sulfonamide | |
| 857 | N-[2-({8-chloro-4-[(3-chloro-4-fluorophenyl)amino]-3- | 0.67 |
| | cyanoquinolin-6-yl}amino)ethyl]-1-pyridin-2- | |
| | ylmethanesulfonamide | |
| 858 | N-[2-({8-chloro-4-[(3-chloro-4-fluorophenyl)amino]-3- | 0.79 |
| | cyanoquinolin-6-yl}amino)ethyl]-4- | |
| | cyanobenzenesulfonamide | |
| 859 | N-[2-({8-chloro-4-[(3-chloro-4-fluorophenyl)amino]-3- | >3 |
| | cyanoquinolin-6-yl}amino)ethyl]pyridine-2-carboxamide | |
| 860 | N-[2-({8-chloro-4-[(3-chloro-4-fluorophenyl)amino]-3- | >3 |
| | cyanoquinolin-6-yl}amino)ethyl]nicotinamide | |
| 861 | N-[2-({8-chloro-4-[(3-chloro-4-fluorophenyl)amino]-3- | >3 |
| | cyanoquinolin-6-yl}amino)ethyl]-2-pyridin-2-ylacetamide | |
| 862 | N-[2-({8-chloro-4-[(3-chloro-4-fluorophenyl)amino]-3- | >3 |
| | cyanoquinolin-6-yl}amino)ethyl]-2-pyridin-3-ylacetamide | |
| 863 | N-[2-({8-chloro-4-[(3-chloro-4-fluorophenyl)amino]-3- | 1.04 |
| | cyanoquinolin-6-yl}amino)ethyl]-2-pyridin-4-ylacetamide | |
| 864 | N-[2-({8-chloro-4-[(3-chloro-4-fluorophenyl)amino]-3- | 0.65 |
| | cyanoquinolin-6-yl}amino)ethyl]pyrimidine-5- | |
| | carboxamide | |
| 865 | N-[(1-acetylpiperidin-4-yl)methyl]-N-{8-chloro-4-[(3- | 0.38 |
| | chloro-4-fluorophenyl)amino]-3-cyanoquinolin-6- | |
| | yl}acetamide | |
| 866 | 6-{[(1-acetylpiperidin-4-yl)methyl]amino}-8-chloro-4-[(3- | 0.26 |
| | chloro-4-fluorophenyl)amino]quinoline-3-carbonitrile | |
| 867 | 8-chloro-4-[(3-chloro-4-fluorophenyl)amino]-6-{[1- | 1.01 |
| | (pyridin-3-ylmethyl)pyrrolidin-3-yl]amino}quinoline-3- | |
| | carbonitrile | |
| 868 | 8-chloro-4-[(3-chloro-4-fluorophenyl)amino]-6-[(1- | 1.19 |
| | ethylpiperidin-3-yl)amino]quinoline-3-carbonitrile | |
| 869 | 8-chloro-4-[(3-chloro-4-fluorophenyl)amino]-6-{[1- | 0.82 |
| | (pyridin-3-ylmethyl)piperidin-4-yl]amino}quinoline-3- | |
| | carbonitrile | |
| 870 | 8-chloro-4-[(3-chloro-4-fluorophenyl)amino]-6-{[(1- | 1.97 |
| | ethylpiperidin-2-yl)methyl]amino}quinoline-3-carbonitrile | |
| 871 | 6-[(1-acetylpyrrolidin-3-yl)amino]-8-chloro-4-[(3-chloro-4- | 0.15 |
| | fluorophenyl)amino]quinoline-3-carbonitrile | |
| 872 | 6-[(1-acetylpiperidin-3-yl)amino]-8-chloro-4-[(3-chloro-4- | 0.096 |
| | fluorophenyl)amino]quinoline-3-carbonitrile | |
| 873 | 6-[(1-acetylpiperidin-4-yl)amino]-8-chloro-4-[(3-chloro-4- | 0.66 |
| | fluorophenyl)amino]quinoline-3-carbonitrile | |
| 874 | 6-{[(1-acetylpiperidin-3-yl)methyl]amino}-8-chloro-4-[(3- | 0.61 |
| | chloro-4-fluorophenyl)amino]quinoline-3-carbonitrile | |
| 875 | 6-{[(1-acetylpiperidin-2-yl)methyl]amino]-8-chloro-4-[(3- | 0.62 |
| | chloro-4-fluorophenyl)amino]quinoline-3-carbonitrile | |
| 876 | 2-{4-[({8-chloro-4-[(3-chloro-4-fluorophenyl)amino]-3- | 0.0013 |
| | cyanoquinolin-6-yl}amino)methyl]-1H-1,2,3-triazol-1-yl}- | |
| | N-cyclopropylacetamide | |
| 877 | 6-({[1-(3-aminopropyl)-1H-1,2,3-triazol-4- | 0.02 |
| | yl]methyl}amino)-8-chloro-4-[(3-chloro-4- | |
| | fluorophenyl)amino]quinoline-3-carbonitrile | |
| 878 | 8-bromo-4-[(3-chloro-4-fluorophenyl)amino]-6-[({1-[(1- | 0.11 |
| | isopropylpyrrolidin-2-yl)methyl]-1H-1,2,3-triazol-4- | |
| | yl}methyl)amino]quinoline-3-carbonitrile | |
| 879 | 8-chloro-4-[(3-chloro-4-fluorophenyl)amino]-6-({[1- | 0.0011 |
| | (pyrimidin-5-ylmethyl)-1H-1,2,3-triazol-4- | |
| | yl]methyl}amino)quinoline-3-carbonitrile | |
| 880 | 8-bromo-4-[(3-chloro-4-fluorophenyl)amino]-6-({[1- | 0.0025 |
| | (pyrimidin-5-ylmethyl)-1H-1,2,3-triazol-4- | |
| | yl]methyl}amino)quinoline-3-carbonitrile | |
| 881 | 8-bromo-4-[(3-chloro-4-fluorophenyl)amino]-6-{[(1-{[6- | 0.27 |
| | (trifluoromethyl)pyridin-2-yl]methyl}-1H-1,2,3-triazol-4- | |
| | yl)methyl]aminol}quinoline-3-carbonitrile | |
| 882 | 8-chloro-4-[(3-chloro-4-fluorophenyl)amino]-6-{[(1-{[6- | 0.27 |
| | (trifluoromethyl)pyridin-2-yl]methyl}-1H-1,2,3-triazol-4- | |
| | yl)methyl]amino}quinoline-3-carbonitrile | |
| 883 | 8-chloro-4-[(3-chloro-4-fluorophenyl)amino]-6-{[(1-{[2- | 0.08 |
| | (trifluoromethyl)pyridin-4-yl]methyl}-1H-1,2,3-triazol-4- | |
| | yl)methyl]amino}quinoline-3-carbonitrile | |
| 884 | ethyl 2-[({8-chloro-4-[(3-chloro-4-fluorophenyl)amino]-3- | 0.66 |
| | cyanoquinolin-6- | |
| | yl}amino)methyl]cyclopropanecarboxylate | |
| 885 | 6-[(2-azepan-1-yl-2-oxoethyl)amino]-8-chloro-4-[(3- | 0.39 |
| | chloro-4-fluorophenyl)amino]quinoline-3-carbonitrile | |
| 886 | 2-[({8-chloro-4-[(3-chloro-4-fluorophenyl)amino]-3- | 0.22 |
| | cyanoquinolin-6-yl}amino)methyl]cyclopropanecarboxylic | |
| | acid | |
| 887 | tert-butyl (2R)-2-({4-[({8-chloro-4-[(3-chloro-4- | 0.16 |
| | fluorophenyl)amino]-3-cyanoquinolin-6-yl}amino)methyl]- | |
| | 1H-1,2,3-triazol-1-yl}methyl)pyrrolidine-1-carboxylate | |
| 888 | tert-butyl (2S)-2-({4-[({8-chloro-4-[(3-chloro-4- | 0.2 |
| | fluorophenyl)amino]-3-cyanoquinolin-6-yl}amino)methyl]- | |
| | 1H-1,2,3-triazol-1-yl}methyl)pyrrolidine-1-carboxylate | |
| 889 | 8-chloro-4-[(3-chloro-4-fluorophenyl)amino]-6-[({1-[(2R)- | 0.0084 |
| | pyrrolidin-2-ylmethyl]-1H-1,2,3-triazol-4- | |
| | yl}methyl)amino]quinoline-3-carbonitrile | |
| 890 | 8-chloro-4-[(3-chloro-4-fluorophenyl)amino]-6-[({1-[(2S)- | 0.0076 |
| | pyrrolidin-2-ylmethyl]-1H-1,2,3-triazol-4- | |
| | yl}methyl)amino]quinoline-3-carbonitrile | |
| 891 | 8-chloro-4-[(3-chloro-4-fluorophenyl)amino]-6-{[(1-{[(2R)- | 0.033 |
| | 1-methylpyrrolidin-2-yl]methyl}-1H-1,2,3-triazol-4- | |
| | yl)methyl]amino}quinoline-3-carbonitrile | |
| 892 | 8-chloro-4-[(3-chloro-4-fluorophenyl)amino]-6-{[(1-{[(2R)- | 0.03 |
| | 1-ethylpyrrolidin-2-yl]methyl}-1H-1,2,3-triazol-4- | |
| | yl)methyl]amino}quinoline-3-carbonitrile | |
| 893 | 8-chloro-4-[(3-chloro-4-fluorophenyl)amino]-6-{[(1-{[(2R)- | 0.046 |
| | 1-isopropylpyrrolidin-2-yl]methyl}-1H-1,2,3-triazol-4- | |
| | yl)methyl]amino}quinoline-3-carbonitrile | |
| 894 | 8-chloro-4-[(3-chloro-4-fluorophenyl)amino]-6-{[(1-{[(2S)- | 0.0018 |
| | 1-methylpyrrolidin-2-yl]methyl}-1H-1,2,3-triazol-4- | |
| | yl)methyl]amino}quinoline-3-carbonitrile | |
| 895 | 8-chloro-4-[(3-chloro-4-fluorophenyl)amino]-6-{[(1-{[(2S)- | 0.01 |
| | 1-ethylpyrrolidin-2-yl]methyl}-1H-1,2,3-triazol-4- | |
| | yl)methyl]amino}quinoline-3-carbonitrile | |
| 896 | 8-chloro-4-[(3-chloro-4-fluorophenyl)amino]-6-{[(1-{[(2S)- | 0.034 |
| | 1-isopropylpyrrolidin-2-yl]methyl}-1H-1,2,3-triazol-4- | |
| | yl)methyl]amino}quinoline-3-carbonitrile | |
| 897 | (2R)-2-({4-[({8-chloro-4-[(3-chloro-4-fluorophenyl)amino]- | 0.11 |
| | 3-cyanoquinolin-6-yl}amino)methyl]-1H-1,2,3-triazol-1- | |
| | yl}methyl)-N,N-dimethylpyrrolidine-1-carboxamide | |
| 898 | 6-{[(1-{[(2R)-1-acetylpyrrolidin-2-yl]methyl}-1H-1,2,3- | 0.0002 |
| | triazol-4-yl)methyl]amino}-8-chloro-4-[(3-chloro-4- | |
| | fluorophenyl)amino]quinoline-3-carbonitrile | |
| 899 | (2R)-2-({4-[({8-chloro-4-[(3-chloro-4-fluorophenyl)amino]- | 0.0006 |
| | 3-cyanoquinolin-6-yl}amino)methyl]-1H-1,2,3-triazol-1- | |
| | yl}methyl)-N-ethylpyrrolidine-1-carboxamide | |
| 900 | (2S)-2-({4-[({8-chloro-4-[(3-chloro-4-fluorophenyl)amino]- | 0.0005 |
| | 3-cyanoquinolin-6-yl}amino)methyl]-1H-1,2,3-triazol-1- | |
| | yl}methyl)-N,N-dimethylpyrrolidine-1-carboxamide | |
| 901 | 6-{[(1-{[(2S)-1-acetylpyrrolidin-2-yl]methyl}-1H-1,2,3- | 0.001 |
| | triazol-4-yl)methyl]amino}-8-chloro-4-[(3-chloro-4- | |
| | fluorophenyl)amino]quinoline-3-carbonitrile | |
| 902 | (2S)-2-({4-[({8-chloro-4-[(3-chloro-4-fluorophenyl)amino]- | 0.0005 |
| | 3-cyanoquinolin-6-yl}amino)methyl]-1H-1,2,3-triazol-1- | |
| | yl}methyl)-N-ethylpyrrolidine-1-carboxamide | |
| 903 | 6-({[1-(azepan-1-ylacetyl)piperidin-3-yl]methyl}amino)-8- | 0.62 |
| | chloro-4-[(3-chloro-4-fluorophenyl)amino]quinoline-3- | |
| | carbonitrile | |
| 904 | 8-chloro-4-[(3-chloro-4-fluorophenyl)amino]-6-[({1-[N-(2- | 0.26 |
| | methoxyethyl)glycyl]piperidin-3- | |
| | yl}methyl)amino]quinoline-3-carbonitrile | |
| 905 | N-(3-{4-[({8-chloro-4-[(3-chloro-4-fluorophenyl)amino]-3- | 0.0028 |
| | cyanoquinolin-6-yl}amino)methyl]-1H-1,2,3-triazol-1- | |
| | yl}propyl)methanesulfonamide | |
| 906 | N-(3-{4-[({8-chloro-4-[(3-chloro-4-fluorophenyl)amino]-3- | 0.0013 |
| | cyanoquinolin-6-yl}amino)methyl]-1H-1,2,3-triazol-1- | |
| | yl}propyl)-1,1,1-trifluoromethanesulfonamide | |
| 907 | N-(3-{4-[({8-chloro-4-[(3-chloro-4-fluorophenyl)amino]-3- | 0.0045 |
| | cyanoquinolin-6-yl}amino)methyl]-1H-1,2,3-triazol-1- | |
| | yl}propyl)propane-2-sulfonamide | |
| 908 | N-(3-{4-[({8-chloro-4-[(3-chloro-4-fluorophenyl)amino]-3- | 0.001 |
| | cyanoquinolin-6-yl}amino)methyl]-1H-1,2,3-triazol-1- | |
| | yl}propyl)-N′-isopropylurea | |
| 909 | N-(3-{4-[({8-chloro-4-[(3-chloro-4-fluorophenyl)amino]-3- | 0.001 |
| | cyanoquinolin-6-yl}amino)methyl]-1H-1,2,3-triazol-1- | |
| | yl}propyl)-N′-methylurea | |
| 910 | N-(3-{4-[({8-chloro-4-[(3-chloro-4-fluorophenyl)amino]-3- | 0.0007 |
| | cyanoquinolin-6-yl}amino)methyl]-1H-1,2,3-triazol-1- | |
| | yl}propyl)acetamide | |
| 911 | N-(3-{4-[({8-chloro-4-[(3-chloro-4-fluorophenyl)amino]-3- | 0.0024 |
| | cyanoquinolin-6-yl}amino)methyl]-1H-1,2,3-triazol-1- | |
| | yl}propyl)-2-methylpropanamide | |
| 912 | N-(3-{4-[({8-chloro-4-[(3-chloro-4-fluorophenyl)amino]-3- | 0.0019 |
| | cyanoquinolin-6-yl}amino)methyl]-1H-1,2,3-triazol-1- | |
| | yl}propyl)cyclopropanecarboxamide | |
| 913 | 8-chloro-4-[(3-chloro-4-fluorophenyl)amino]-6-[({1-[3- | 0.045 |
| | (diisobutylamino)propyl]-1H-1,2,3-triazol-4- | |
| | yl}methyl)amino]quinoline-3-carbonitrile | |
| 914 | 8-chloro-4-[(3-chloro-4-fluorophenyl)amino]-6-[({1-[3- | 0.052 |
| | (cyclopentylamino)propyl]-1H-1,2,3-triazol-4- | |
| | yl}methyl)amino]quinoline-3-carbonitrile | |
| 915 | N-{8-chloro-4-[(3-chloro-4-fluorophenyl)amino]-3- | 0.17 |
| | cyanoquinolin-6-yl}-N-({1-[3-(formylamino)propyl]-1H- | |
| | 1,2,3-triazol-4-yl}methyl)formamide | |
| 916 | N-[3-(4-{[{8-chloro-4-[(3-chloro-4-fluorophenyl)amino]-3- | 0.83 |
| | cyanoquinolin-6-yl}(formyl)amino]methyl}-1H-1,2,3- | |
| | triazol-1-yl)propyl]methanesulfonamide | |
| 917 | 8-bromo-4-[(3-chloro-4-fluorophenyl)amino]-6-({[1- | 0.076 |
| | (methoxymethyl)-1H-1,2,3-triazol-4- | |
| | yl]methyl}amino)quinoline-3-carbonitrile | |
| 918 | 8-chloro-4-[(3-chloro-4-fluorophenyl)amino]-6-({[1-(2- | 0.0009 |
| | pyridin-4-ylethyl)-1H-1,2,3-triazol-4- | |
| | yl]methyl}amino)quinoline-3-carbonitrile | |
| 919 | 8-bromo-4-[(3-chloro-4-fluorophenyl)amino]-6-({[1-(2- | 0.0013 |
| | pyridin-4-ylethyl)-1H-1,2,3-triazol-4- | |
| | yl]methyl}amino)quinoline-3-carbonitrile | |
| 920 | 8-chloro-4-[(3-chloro-4-fluorophenyl)amino]-6-{[(1-{N-[2- | 0.86 |
| | (dimethylamino)ethyl]glycyl}piperidin-3- | |
| | yl)methyl]amino}quinoline-3-carbonitrile | |
| 921 | 8-chloro-4-[(3-chloro-4-fluorophenyl)amino]-6-[({1-[N- | 0.54 |
| | (pyridin-3-ylmethyl)glycyl]piperidin-3- | |
| | yl}methyl)amino]quinoline-3-carbonitrile | |
| 922 | 8-chloro-4-[(3-chloro-4-fluorophenyl)amino]-6-[({1-[N- | 1.69 |
| | (pyridin-2-ylmethyl)glycyl]piperidin-3- | |
| | yl}methyl)amino]quinoline-3-carbonitrile | |
| 923 | 8-chloro-4-[(3-chloro-4-fluorophenyl)amino]-6-({[1-(N- | 0.56 |
| | pyridin-3-ylglycyl)piperidin-3-yl]methyl}amino)quinoline- | |
| | 3-carbonitrile | |
| 924 | 8-chloro-6-({[1-(chloroacetyl)piperidin-3- | 1.32 |
| | yl]methyl}amino)-4-[(3-chloro-4- | |
| | fluorophenyl)amino]quinoline-3-carbonitrile | |
| 925 | tert-butyl 4-{4-[({8-chloro-4-[(3-chloro-4- | 0.073 |
| | fluorophenyl)amino]-3-cyanoquinolin-6-yl}amino)methyl]- | |
| | 1H-1,2,3-triazol-1-yl}piperidine-1-carboxylate | |
| 926 | 8-chloro-4-[(3-chloro-4-fluorophenyl)amino]-6-{[(1- | 0.0053 |
| | piperidin-4-yl-1H-1,2,3-triazol-4- | |
| | yl)methyl]amino}quinoline-3-carbonitrile | |
| 927 | 8-chloro-4-[(3-chloro-4-fluorophenyl)amino]-6-({[1-(1- | 0.0022 |
| | methylpiperidin-4-yl)-1H-1,2,3-triazol-4- | |
| | yl]methyl}amino)quinoline-3-carbonitrile | |
| 928 | 8-chloro-4-[(3-chloro-4-fluorophenyl)amino]-6-({[1-(1- | 0.0091 |
| | isopropylpiperidin-4-yl)-1H-1,2,3-triazol-4- | |
| | yl]methyl}amino)quinoline-3-carbonitrile | |
| 929 | 4-{4-[({8-chloro-4-[(3-chloro-4-fluorophenyl)amino]-3- | 0.007 |
| | cyanoquinolin-6-yl}amino)methyl]-1H-1,2,3-triazol-1-yl}- | |
| | N,N-dimethylpiperidine-1-carboxamide | |
| 930 | 6-({[1-(1-acetylpiperidin-4-yl)-1H-1,2,3-triazol-4- | 0.0033 |
| | yl]methyl}amino)-8-chloro-4-[(3-chloro-4- | |
| | fluorophenyl)amino]quinoline-3-carbonitrile | |
| 931 | 4-{4-[({8-chloro-4-[(3-chloro-4-fluorophenyl)amino]-3- | 0.0189 |
| | cyanoquinolin-6-yl}amino)methyl]-1H-1,2,3-triazol-1-yl}- | |
| | N-ethylpiperidine-1-carboxamide | |
| 932 | 2,5-anhydro-1-({8-chloro-4-[(3-chloro-4- | 0.41 |
| | fluorophenyl)amino]-3-cyanoquinolin-6-yl}amino)-1,3- | |
| | dideoxy-D-erythro-pentitol | |
| 933 | 8-chloro-4-[(3-chloro-4-fluorophenyl)amino]-6-{[(1R)-1- | 1.99 |
| | methyl-2-(pyridin-2-ylamino)ethyl]amino}quinoline-3- | |
| | carbonitrile | |
| 934 | 8-chloro-4-[(3-chloro-4-fluorophenyl)amino]-6-({[1-(2- | 0.018 |
| | oxotetrahydrofuran-3-yl)-1H-1,2,3-triazol-4- | |
| | yl]methyl}amino)quinoline-3-carbonitrile | |
| 935 | 2,5-anhydro-4-O-benzyl-1-({8-chloro-4-[(3-chloro-4- | >3 |
| | fluorophenyl)amino]-3-cyanoquinolin-6-yl}amino)-1,3- | |
| | dideoxy-D-erythro-pentitol | |
| 936 | 8-chloro-4-[(3-chloro-4-fluorophenyl)amino]-6-{[(1-{2-[1- | 0.13 |
| | (cyclopropylmethyl)piperidin-4-yl]ethyl}-1H-1,2,3-triazol- | |
| | 4-yl)methyl]amino}quinoline-3-carbonitrile | |
| 937 | 8-chloro-4-[(3-chloro-4-fluorophenyl)amino]-6-[({1-[2-(1- | 0.15 |
| | cyclobutylpiperidin-4-yl)ethyl]-1H-1,2,3-triazol-4- | |
| | yl}methyl)amino]quinoline-3-carbonitrile | |
| 938 | 8-chloro-4-[(3-chloro-4-fluorophenyl)amino]-6-[({1-[2-(1- | 0.2 |
| | cyclopentylpiperidin-4-yl)ethyl]-1H-1,2,3-triazol-4- | |
| | yl}methyl)amino]quinoline-3-carbonitrile | |
| 939 | 2,5-anhydro-1-({8-chloro-4-[(3-chloro-4- | >3 |
| | fluorophenyl)amino]-3-cyanoquinolin-6-yl}amino)-1,3- | |
| | dideoxy-4-O-(pyridin-3-ylmethyl)-D-erythro-pentitol | |
| 940 | 8-chloro-4-[(3-chloro-4-fluorophenyl)amino]-6-[(1- | 0.15 |
| | piperidin-4-yl-1H-1,2,3-triazol-4-yl)methoxy]quinoline-3- | |
| | carbonitrile | |
| 941 | tert-butyl 4-{4-[({8-chloro-4-[(3-chloro-4- | 0.52 |
| | fluorophenyl)amino]-3-cyanoquinolin-6-yl}oxy)methyl]- | |
| | 1H-1,2,3-triazol-1-yl}piperidine-1-carboxylate | |
| 942 | 8-bromo-4-[(3-chloro-4-fluorophenyl)amino]-6-{[(1-{2- | 0.019 |
| | [(2S)-1-isopropylpyrrolidin-2-yl]ethyl}-1H-1,2,3-triazol-4- | |
| | yl)methyl]amino}quinoline-3-carbonitrile | |
| 943 | tert-butyl 4-[4-({[8-chloro-3-cyano-4- | >3 |
| | (cyclopentylamino)quinolin-6-yl]amino}methyl)-1H-1,2,3- | |
| | triazol-1-yl]piperidine-1-carboxylate | |
| 944 | 8-chloro-4-(cyclopentylamino)-6-[({1-[2-(1- | >3 |
| | methylpyrrolidin-2-yl)ethyl]-1H-1,2,3-triazol-4- | |
| | yl}methyl)amino]quinoline-3-carbonitrile | |
| 945 | 6-({[1-(2-azepan-1-ylethyl)-1H-1,2,3-triazol-4- | 0.63 |
| | yl]methyl}amino)-8-chloro-4- | |
| | (cyclopentylamino)quinoline-3-carbonitrile | |
| 946 | 8-chloro-4-(cyclopentylamino)-6-[({1-[2- | 0.59 |
| | (dimethylamino)ethyl]-1H-1,2,3-triazol-4- | |
| | yl}methyl)amino]quinoline-3-carbonitrile | |
| 947 | 8-chloro-4-(cycloheptylamino)-6-[({1-[2-(1- | 0.70 |
| | methylpyrrolidin-2-yl)ethyl]-1H-1,2,3-triazol-4- | |
| | yl}methyl)amino]quinoline-3-carbonitrile | |
| 948 | tert-butyl 4-[4-({[8-chloro-3-cyano-4- | 2.31 |
| | (cycloheptylamino)quinolin-6-yl]amino}methyl)-1H-1,2,3- | |
| | triazol-1-yl]piperidine-1-carboxylate | |
| 949 | 6-({[1-(2-azepan-1-ylethyl)-1H-1,2,3-triazol-4- | 0.46 |
| | yl]methyl}amino)-8-chloro-4- | |
| | (cycloheptylamino)quinoline-3-carbonitrile | |
| 950 | 8-chloro-4-(cycloheptylamino)-6-[({1-[2- | 0.62 |
| | (dimethylamino)ethyl]-1H-1,2,3-triazol-4- | |
| | yl}methyl)amino]quinoline-3-carbonitrile | |
| 951 | 8-chloro-4-(cyclobutylamino)-6-[({1-[2-(1- | 0.46 |
| | methylpyrrolidin-2-yl)ethyl]-1H-1,2,3-triazol-4- | |
| | yl}methyl)amino]quinoline-3-carbonitrile | |
| 952 | 8-chloro-6-[({1-[2-(1-methylpyrrolidin-2-yl)ethyl]-1H- | 1.13 |
| | 1,2,3-triazol-4-yl}methyl)amino]-4-(tetrahydro-2H-pyran- | |
| | 4-ylamino)quinoline-3-carbonitrile | |
| 953 | tert-butyl 4-[4-({[8-chloro-3-cyano-4- | 0.81 |
| | (cyclobutylamino)quinolin-6-yl]amino}methyl)-1H-1,2,3- | |
| | triazol-1-yl]piperidine-1-carboxylate | |
| 954 | 8-chloro-4-(cyclobutylamino)-6-({[1-(2-piperidin-1- | 0.52 |
| | ylethyl)-1H-1,2,3-triazol-4-yl]methyl}amino)quinoline-3- | |
| | carbonitrile | |
| 955 | 6-({[1-(2-azepan-1-ylethyl)-1H-1,2,3-triazol-4- | 0.44 |
| | yl]methyl}amino)-8-chloro-4-(cyclobutylamino)quinoline- | |
| | 3-carbonitrile | |
| 956 | 8-chloro-4-(cyclobutylamino)-6-[({1-[2- | 0.73 |
| | (dimethylamino)ethyl]-1H-1,2,3-triazol-4- | |
| | yl}methyl)amino]quinoline-3-carbonitrile | |
| 957 | tert-butyl 4-(2-{4-[({8-chloro-4-[(3-chloro-4- | >3 |
| | fluorophenyl)amino]-3-cyanoquinolin-6-yl}amino)methyl]- | |
| | 1H-1,2,3-triazol-1-yl}ethyl)piperazine-1-carboxylate | |
| 958 | 8-chloro-4-[(3-chloro-4-fluorophenyl)amino]-6-({[1-(2- | 0.31 |
| | piperazin-1-ylethyl)-1H-1,2,3-triazol-4- | |
| | yl]methyl}amino)quinoline-3-carbonitrile | |
| 959 | 6-[({1-[2-(4-acetylpiperazin-1-yl)ethyl]-1H-1,2,3-triazol-4- | 0.092 |
| | yl}methyl)amino]-8-chloro-4-[(3-chloro-4- | |
| | fluorophenyl)amino]quinoline-3-carbonitrile | |
| 960 | 8-chloro-4-[(3-chloro-4-fluorophenyl)amino]-6-{[(1-{2-[4- | 0.13 |
| | (3,3-dimethylbutanoyl)piperazin-1-yl]ethyl}-1H-1,2,3- | |
| | triazol-4-yl)methyl]amino}quinoline-3-carbonitrile | |
| 961 | 4-(2-{4-[({8-chloro-4-[(3-chloro-4-fluorophenyl)amino]-3- | 0.031 |
| | cyanoquinolin-6-yl}amino)methyl]-1H-1,2,3-triazol-1- | |
| | yl}ethyl)-N,N-dimethylpiperazine-1-carboxamide | |
| 962 | 4-(2-{4-[({8-chloro-4-[(3-chloro-4-fluorophenyl)amino]-3- | 0.023 |
| | cyanoquinolin-6-yl}amino)methyl]-1H-1,2,3-triazol-1- | |
| | yl}ethyl)-N-ethylpiperazine-1-carboxamide | |
| 963 | 8-chloro-4-[(3-chloro-4-fluorophenyl)amino]-6-[({1-[2-(4- | 0.65 |
| | methylpiperazin-1-yl)ethyl]-1H-1,2,3-triazol-4- | |
| | yl}methyl)amino]quinoline-3-carbonitrile | |
| 964 | 8-chloro-4-[(3-chloro-4-fluorophenyl)amino]-6-[({1-[2-(4- | 0.26 |
| | ethylpiperazin-1-yl)ethyl]-1H-1,2,3-triazol-4- | |
| | yl}methyl)amino]quinoline-3-carbonitrile | |
| 965 | 8-chloro-4-[(3-chloro-4-fluorophenyl)amino]-6-[({1-[2-(4- | 0.43 |
| | isopropylpiperazin-1-yl)ethyl]-1H-1,2,3-triazol-4- | |
| | yl}methyl)amino]quinoline-3-carbonitrile | |
| 966 | 8-chloro-4-[(3-chloro-4-fluorophenyl)amino]-6-{[(1-{2-[4- | 0.15 |
| | (cyclopropylmethyl)piperazin-1-yl]ethyl}-1H-1,2,3-triazol- | |
| | 4-yl)methyl]amino}quinoline-3-carbonitrile | |
| 967 | 4-(cyclohexylamino)-6-[({1-[2-(1-methylpyrrolidin-2- | 0.46 |
| | yl)ethyl]-1H-1,2,3-triazol-4-yl}methyl)amino]quinoline-3- | |
| | carbonitrile | |
| 968 | tert-butyl 4-[4-({[3-cyano-4-(cyclohexylamino)quinolin-6- | 0.67 |
| | yl]amino}methyl)-1H-1,2,3-triazol-1-yl]piperidine-1- | |
| | carboxylate | |
| 969 | 4-(cyclohexylamino)-6-[({1-[2-(dimethylamino)ethyl]-1H- | 0.38 |
| | 1,2,3-triazol-4-yl}methyl)amino]quinoline-3-carbonitrile | |
| 970 | 6-({[1-(2-azepan-1-ylethyl)-1H-1,2,3-triazol-4- | 0.51 |
| | yl]methyl}amino)-4-(cyclohexylamino)quinoline-3- | |
| | carbonitrile | |
| 971 | 8-chloro-6-[({1-[2-(1-methylpyrrolidin-2-yl)ethyl]-1H- | >3 |
| | 1,2,3-triazol-4-yl}methyl)amino]-4-(spiro[5.5]undec-3- | |
| | ylamino)quinoline-3-carbonitrile | |
| 972 | 8-chloro-6-[({1-[2-(1-methylpyrrolidin-2-yl)ethyl]-1H- | >3 |
| | 1,2,3-triazol-4-yl}methyl)amino]-4-(spiro[4.5]dec-7- | |
| | ylamino)quinoline-3-carbonitrile | |
| 973 | tert-butyl 4-[4-({[8-chloro-3-cyano-4-(spiro[5.5]undec-3- | >3 |
| | ylamino)quinolin-6-yl]amino}methyl)-1H-1,2,3-triazol-1- | |
| | yl]piperidine-1-carboxylate | |
| 974 | tert-butyl 4-[4-({[8-chloro-3-cyano-4-(spiro[4.5]dec-7- | >3 |
| | ylamino)quinolin-6-yl]amino}methyl)-1H-1,2,3-triazol-1- | |
| | yl]piperidine-1-carboxylate | |
| 975 | 6-({[1-(2-azepan-1-ylethyl)-1H-1,2,3-triazol-4- | >3 |
| | yl]methyl}amino)-8-chloro-4-(spiro[5.5]undec-3- | |
| | ylamino)quinoline-3-carbonitrile | |
| 976 | 6-({[1-(2-azepan-1-ylethyl)-1H-1,2,3-triazol-4- | >3 |
| | yl]methyl}amino)-8-chloro-4-(spiro[4.5]dec-7- | |
| | ylamino)quinoline-3-carbonitrile | |
| 977 | 4-(cyclohexylamino)-6-{[(1-piperidin-4-yl-1H-1,2,3- | 0.13 |
| | triazol-4-yl)methyl]amino}quinoline-3-carbonitrile | |
| 978 | 8-chloro-6-{[(1-piperidin-4-yl-1H-1,2,3-triazol-4- | >3 |
| | yl)methyl]amino}-4-(spiro[5.5]undec-3-ylamino)quinoline- | |
| | 3-carbonitrile | |
| 979 | 8-chloro-6-{[(1-piperidin-4-yl-1H-1,2,3-triazol-4- | 0.97 |
| | yl)methyl]amino}-4-(spiro[4.5]dec-7-ylamino)quinoline-3- | |
| | carbonitrile | |
| 980 | methyl 4-{4-[({8-chloro-4-[(3-chloro-4- | 0.024 |
| | fluorophenyl)amino]-3-cyanoquinolin-6-yl}amino)methyl]- | |
| | 1H-1,2,3-triazol-1-yl}piperidine-1-carboxylate | |
| 981 | methyl 4-(4-{[{8-chloro-4-[(3-chloro-4- | 0.61 |
| | fluorophenyl)amino]-3-cyanoquinolin-6- | |
| | yl}(methoxycarbonyl)amino]methyl}-1H-1,2,3-triazol-1- | |
| | yl)piperidine-1-carboxylate | |
| 982 | 2-fluoroethyl 4-{4-[({8-chloro-4-[(3-chloro-4- | 0.031 |
| | fluorophenyl)amino]-3-cyanoquinolin-6-yl}amino)methyl]- | |
| | 1H-1,2,3-triazol-1-yl}piperidine-1-carboxylate | |
| 983 | 2-fluoroethyl 4-[4-({{8-chloro-4-[(3-chloro-4- | >3 |
| | fluorophenyl)amino]-3-cyanoquinolin-6-yl}[(2- | |
| | fluoroethoxy)carbonyl]amino}methyl)-1H-1,2,3-triazol-1- | |
| | yl]piperidine-1-carboxylate | |
| 984 | 8-chloro-6-[({1-[2-(dimethylamino)ethyl]-1H-1,2,3-triazol- | 1.09 |
| | 4-yl}methyl)amino]-4-[(2,2,2- | |
| | trifluoroethyl)amino]quinoline-3-carbonitrile | |
| 985 | 8-chloro-6-({[1-(2-piperidin-1-ylethyl)-1H-1,2,3-triazol-4- | 1.41 |
| | yl]methyl}amino)-4-[(2,2,2-trifluoroethyl)amino]quinoline- | |
| | 3-carbonitrile | |
| 986 | 8-chloro-4-(cyclobutylamino)-6-{[(1-piperidin-4-yl-1H- | 0.35 |
| | 1,2,3-triazol-4-yl)methyl]amino}quinoline-3-carbonitrile | |
| 987 | 8-chloro-6-{[(1-piperidin-4-yl-1H-1,2,3-triazol-4- | 1.1 |
| | yl)methyl]amino}-4-(tetrahydro-2H-pyran-4- | |
| | ylamino)quinoline-3-carbonitrile | |
| 988 | 6-({[1-(2-azepan-1-ylethyl)-1H-1,2,3-triazol-4- | >3 |
| | yl]methyl}amino)-8-chloro-4-[(4- | |
| | phenoxyphenyl)amino]quinoline-3-carbonitrile | |
| 989 | tert-butyl 4-{4-[({8-chloro-3-cyano-4-[(4- | 0.4 |
| | phenoxyphenyl)amino]quinolin-6-yl}amino)methyl]-1H- | |
| | 1,2,3-triazol-1-yl}piperidine-1-carboxylate | |
| 990 | 8-chloro-6-[({1-[2-(1-methylpyrrolidin-2-yl)ethyl]-1H- | 0.83 |
| | 1,2,3-triazol-4-yl}methyl)amino]-4-[(4- | |
| | phenoxyphenyl)amino]quinoline-3-carbonitrile | |
| 991 | 8-chloro-4-[(3-chloro-4-fluorophenyl)amino]-6-{[(1-{1- | 0.0082 |
| | [(2R,3R)-2,3,4-trihydroxybutyl]piperidin-4-yl}-1H-1,2,3- | |
| | triazol-4-yl)methyl]amino}quinoline-3-carbonitrile | |
| 992 | 8-chloro-4-[(3-chloro-4-fluorophenyl)amino]-6-[({1-[1-(2- | 0.002 |
| | hydroxyethyl)piperidin-4-yl]-1H-1,2,3-triazol-4- | |
| | yl}methyl)amino]quinoline-3-carbonitrile | |
| 993 | 8-chloro-4-[(3-chloro-4-fluorophenyl)amino]-6-({[1-(2- | 0.048 |
| | hydroxy-2-pyridin-2-ylethyl)-1H-1,2,3-triazol-4- | |
| | yl]methyl}amino)quinoline-3-carbonitrile | |
| 994 | 8-chloro-4-[(3-chloro-4-fluorophenyl)amino]-6-[({1-[1- | 0.0068 |
| | (2,3-dihydroxypropyl)piperidin-4-yl]-1H-1,2,3-triazol-4- | |
| | yl}methyl)amino]quinoline-3-carbonitrile | |
| 995 | 6-{[(1-but-2-yn-1-yl-1H-1,2,3-triazol-4-yl)methyl]amino}- | 0.24 |
| | 8-chloro-4-[(3-chloro-4-fluorophenyl)amino]quinoline-3- | |
| | carbonitrile | |
| 996 | 8-chloro-4-[(3-chloro-4-fluorophenyl)amino]-6-{[(5- | >3 |
| | methylisoxazol-3-yl)methyl]amino}quinoline-3- | |
| | carbonitrile | |
| 997 | 6-({[1-(8-azabicyclo[3.2.1]oct-3-yl)-1H-1,2,3-triazol-4- | 0.011 |
| | yl]methyl}amino)-8-chloro-4-[(3-chloro-4- | |
| | fluorophenyl)amino]quinoline-3-carbonitrile | |
| 998 | 8-chloro-4-[(3-chloro-4-fluorophenyl)amino]-6-[({1-[1- | 0.022 |
| | (cyanomethyl)piperidin-4-yl]-1H-1,2,3-triazol-4- | |
| | yl}methyl)amino]quinoline-3-carbonitrile | |
| 999 | 8-chloro-4-(cyclobutylamino)-6-({[1-(1-isopropylpiperidin- | 0.13 |
| | 4-yl)-1H-1,2,3-triazol-4-yl]methyl}amino)quinoline-3- | |
| | carbonitrile | |
| 1000 | 8-chloro-6-({[1-(1-isopropylpiperidin-4-yl)-1H-1,2,3- | 0.48 |
| | triazol-4-yl]methyl}amino)-4-(tetrahydro-2H-pyran-4- | |
| | ylamino)quinoline-3-carbonitrile | |
| 1001 | 8-chloro-4-[(3-chloro-4-fluorophenyl)amino]-6-({[1-(2- | 0.019 |
| | hydroxy-2-pyridin-4-ylethyl)-1H-1,2,3-triazol-4- | |
| | yl]methyl}amino)quinoline-3-carbonitrile | |
| 1002 | 8-chloro-4-[(3-chloro-4-fluorophenyl)amino]-6-[(2- | >3 |
| | thienylmethyl)amino]quinoline-3-carbonitrile | |
| 1003 | 8-chloro-4-[(3-chloro-4-fluorophenyl)amino]-6-({[1-(1- | 0.017 |
| | cyclopropylpiperidin-4-yl)-1H-1,2,3-triazol-4- | |
| | yl]methyl}amino)quinoline-3-carbonitrile | |
| 1004 | 6-{[(4-bromo-2-thienyl)methyl]amino}-8-chloro-4-[(3- | >3 |
| | chloro-4-fluorophenyl)amino]quinoline-3-carbonitrile | |
| 1005 | 8-chloro-4-[(3-chloro-4-fluorophenyl)amino]-6-({[1-(8- | 0.041 |
| | ethyl-8-azabicyclo[3.2.1]oct-3-yl)-1H-1,2,3-triazol-4- | |
| | yl]methyl}amino)quinoline-3-carbonitrile | |
| 1006 | tert-butyl 4-{4-[({8-chloro-4-[(3-chloro-4- | 0.24 |
| | fluorophenyl)amino]-3-cyanoquinolin-6-yl}amino)methyl]- | |
| | 1H-1,2,3-triazol-1-yl}azepane-1-carboxylate | |
| 1007 | 6-{[(1-azepan-4-yl-1H-1,2,3-triazol-4-yl)methyl]amino}-8- | 0.016 |
| | chloro-4-[(3-chloro-4-fluorophenyl)amino]quinoline-3- | |
| | carbonitrile | |
| 1008 | 8-chloro-4-[(3-chloro-4-fluorophenyl)amino]-6-{[(1-{1-[3- | 0.49 |
| | (trifluoromethyl)pyridin-2-yl]piperidin-4-yl}-1H-1,2,3- | |
| | triazol-4-yl)methyl]amino}quinoline-3-carbonitrile | |
| 1009 | 8-chloro-4-[(3-chloro-4-fluorophenyl)amino]-6-[({1-[1- | 0.27 |
| | (2,2,2-trifluoroethyl)azepan-4-yl]-1H-1,2,3-triazol-4- | |
| | yl}methyl)amino]quinoline-3-carbonitrile | |
| 1010 | 8-chloro-4-[(3-chloro-4-fluorophenyl)amino]-6-[({1-[1- | 0.31 |
| | (2,2-difluoroethyl)azepan-4-yl]-1H-1,2,3-triazol-4- | |
| | yl}methyl)amino]quinoline-3-carbonitrile | |
| 1011 | 8-chloro-4-[(3-chloro-4-fluorophenyl)amino]-6-[({1-[1-(2- | 0.0091 |
| | fluoroethyl)azepan-4-yl]-1H-1,2,3-triazol-4- | |
| | yl}methyl)amino]quinoline-3-carbonitrile | |
| 1012 | 8-chloro-4-[(3-chloro-4-fluorophenyl)amino]-6-[({1-[1- | >3 |
| | (2,2,3,3,3-pentafluoropropyl)azepan-4-yl]-1H-1,2,3- | |
| | triazol-4-yl}methyl)amino]quinoline-3-carbonitrile | |
| 1013 | 8-chloro-4-[(3-chloro-4-fluorophenyl)amino]-6-[({1-[1- | 0.43 |
| | (2,2,3,3-tetrafluoropropyl)azepan-4-yl]-1H-1,2,3-triazol- | |
| | 4-yl}methyl)amino]quinoline-3-carbonitrile | |
| 1014 | 8-chloro-4-[(3-chloro-4-fluorophenyl)amino]-6-[({1-[1- | 0.28 |
| | (2,2,2-trifluoroethyl)piperidin-4-yl]-1H-1,2,3-triazol-4- | |
| | yl}methyl)amino]quinoline-3-carbonitrile | |
| 1015 | 8-chloro-4-[(3-chloro-4-fluorophenyl)amino]-6-[({1-[1- | 0.092 |
| | (2,2-difluoroethyl)piperidin-4-yl]-1H-1,2,3-triazol-4- | |
| | yl}methyl)amino]quinoline-3-carbonitrile | |
| 1016 | 8-chloro-4-[(3-chloro-4-fluorophenyl)amino]-6-[({1-[1-(2- | 0.015 |
| | fluoroethyl)piperidin-4-yl]-1H-1,2,3-triazol-4- | |
| | yl}methyl)amino]quinoline-3-carbonitrile | |
| 1017 | 8-chloro-4-[(3-chloro-4-fluorophenyl)amino]-6-[({1-[1- | 1.04 |
| | (2,2,3,3,3-pentafluoropropyl)piperidin-4-yl]-1H-1,2,3- | |
| | triazol-4-yl}methyl)amino]quinoline-3-carbonitrile | |
| 1018 | 8-chloro-4-[(3-chloro-4-fluorophenyl)amino]-6-[({1-[1- | 0.40 |
| | (2,2,3,3-tetrafluoropropyl)piperidin-4-yl]-1H-1,2,3-triazol- | |
| | 4-yl}methyl)amino]quinoline-3-carbonitrile | |
| 1019 | 8-chloro-4-[(3-chloro-4-fluorophenyl)amino]-6-[({5-[2,2,2- | >3 |
| | trifluoro-1-hydroxy-1-(trifluoromethyl)ethyl]pyridin-3- | |
| | yl}methyl)amino]quinoline-3-carbonitrile | |
| 1020 | 8-chloro-4-[(3-chloro-4-fluorophenyl)amino]-6-({[1-(2- | 0.13 |
| | pyridin-3-ylethyl)-1H-1,2,3-triazol-4- | |
| | yl]methyl}amino)quinoline-3-carbonitrile | |
| 1021 | 8-chloro-4-[(3-chloro-4-fluorophenyl)amino]-6-({[1- | 0.43 |
| | (cyanomethyl)-1H-1,2,3-triazol-4- | |
| | yl]methyl}amino)quinoline-3-carbonitrile | |
| 1022 | 8-chloro-4-[(3-chloro-4-fluorophenyl)amino]-6-[({4-[3- | 0.98 |
| | (dimethylamino)propyl]-2- | |
| | thienyl}methyl)amino]quinoline-3-carbonitrile | |
| 1023 | 8-chloro-4-[(3-chloro-4-fluorophenyl)amino]-6-[({4-[3- | 1.16 |
| | (dimethylamino)prop-1-yn-1-yl]-2- | |
| | thienyl}methyl)amino]quinoline-3-carbonitrile | |
| 1024 | 6-{[(4-bromo-2-furyl)methyl]amino}-8-chloro-4-[(3-chloro- | >3 |
| | 4-fluorophenyl)amino]quinoline-3-carbonitrile | |
| 1025 | 8-chloro-4-[(3-chloro-4-fluorophenyl)amino]-6-({[1-({5- | 0.91 |
| | [2,2,2-trifluoro-1-hydroxy-1-(trifluoromethyl)ethyl]pyridin- | |
| | 3-yl}methyl)-1H-1,2,3-triazol-4- | |
| | yl]methyl}amino)quinoline-3-carbonitrile | |
| 1026 | 8-chloro-4-[(3-chloro-4-fluorophenyl)amino]-6-{[(1- | 0.29 |
| | methyl-1H-pyrazol-4-yl)methyl]amino}quinoline-3- | |
| | carbonitrile | |
| 1027 | 8-chloro-4-[(3-chloro-4-fluorophenyl)amino]-6-[({1-[1- | 0.0079 |
| | (cyclopropylmethyl)piperidin-4-yl]-1H-1,2,3-triazol-4- | |
| | yl}methyl)amino]quinoline-3-carbonitrile | |
| 1028 | 8-chloro-4-[(3-chloro-4-fluorophenyl)amino]-6- | 0.41 |
| | [(cyclopropylmethyl)({1-[1-(cyclopropylmethyl)piperidin- | |
| | 4-yl]-1H-1,2,3-triazol-4-yl}methyl)amino]quinoline-3- | |
| | carbonitrile | |
| 1029 | 8-chloro-4-[(3-chloro-4-fluorophenyl)amino]-6- | 0.29 |
| | (cyclobutyl{[1-(1-cyclobutylpiperidin-4-yl)-1H-1,2,3- | |
| | triazol-4-yl]methyl}amino)quinoline-3-carbonitrile | |
| 1030 | 8-chloro-4-[(3-chloro-4-fluorophenyl)amino]-6-({[1-(1- | 0.012 |
| | cyclobutylazepan-4-yl)-1H-1,2,3-triazol-4- | |
| | yl]methyl}amino)quinoline-3-carbonitrile | |
| 1031 | tert-butyl 4-{5-[({8-chloro-4-[(3-chloro-4- | 0.16 |
| | fluorophenyl)amino]-3-cyanoquinolin-6- | |
| | yl}amino)methyl]pyridin-3-yl}-4-hydroxypiperidine-1- | |
| | carboxylate | |
| 1032 | 8-chloro-4-[(3-chloro-4-fluorophenyl)amino]-6-({[5-(4- | >3 |
| | hydroxypiperidin-4-yl)pyridin-3- | |
| | yl]methyl}amino)quinoline-3-carbonitrile | |
| 1033 | 8-chloro-4-[(3-chloro-4-fluorophenyl)amino]-6-({[5-(4- | 0.5 |
| | hydroxy-1-isopropylpiperidin-4-yl)pyridin-3- | |
| | yl]methyl}amino)quinoline-3-carbonitrile | |
| 1034 | 8-chloro-4-[(3-chloro-4-fluorophenyl)amino]-6-({[5-(1- | 0.4 |
| | ethyl-4-hydroxypiperidin-4-yl)pyridin-3- | |
| | yl]methyl}amino)quinoline-3-carbonitrile | |
| 1035 | 8-chloro-4-[(3-chloro-4-fluorophenyl)amino]-6-[({1-[4- | 0.036 |
| | (ethylamino)cyclohexyl]-1H-1,2,3-triazol-4- | |
| | yl}methyl)amino]quinoline-3-carbonitrile | |
| 1036 | 8-chloro-4-[(3-chloro-4-fluorophenyl)amino]-6-[({1-[4- | 0.04 |
| | (methylamino)cyclohexyl]-1H-1,2,3-triazol-4- | |
| | yl}methyl)amino]quinoline-3-carbonitrile | |
| 1037 | 8-chloro-4-[(3-chloro-4-fluorophenyl)amino]-6-[({1-[4- | 0.02 |
| | (isopropylamino)cyclohexyl]-1H-1,2,3-triazol-4- | |
| | yl}methyl)amino]quinoline-3-carbonitrile | |
| 1038 | 8-chloro-4-[(3-chloro-4-fluorophenyl)amino]-6-[({1-[4- | 0.017 |
| | (cyclopentylamino)cyclohexyl]-1H-1,2,3-triazol-4- | |
| | yl}methyl)amino]quinoline-3-carbonitrile | |
| 1039 | 8-chloro-4-[(3-chloro-4-fluorophenyl)amino]-6-[({1-[4- | 0.1 |
| | (pyridin-2-ylamino)cyclohexyl]-1H-1,2,3-triazol-4- | |
| | yl}methyl)amino]quinoline-3-carbonitrile | |
| 1040 | 8-chloro-4-[(3-chloro-4-fluorophenyl)amino]-6-[({1-[4- | 0.017 |
| | (cyclopropylamino)cyclohexyl]-1H-1,2,3-triazol-4- | |
| | yl}methyl)amino]quinoline-3-carbonitrile | |
| 1041 | 8-chloro-4-[(3-chloro-4-fluorophenyl)amino]-6-({[1-(4- | 0.0057 |
| | hydroxycyclohexyl)-1H-1,2,3-triazol-4- | |
| | yl]methyl}amino)quinoline-3-carbonitrile | |
| 1042 | 8-chloro-4-[(3-chloro-4-fluorophenyl)amino]-6-({[1-(2-{5- | 0.13 |
| | [2,2,2-trifluoro-1-hydroxy-1-(trifluoromethyl)ethyl]pyridin- | |
| | 3-yl}ethyl)-1H-1,2,3-triazol-4-yl]methyl}amino)quinoline- | |
| | 3-carbonitrile | |
| 1043 | 8-chloro-4-[(3-chloro-4-fluorophenyl)amino]-6-({[1- | 0.052 |
| | (1,2,2,6,6-pentamethylpiperidin-4-yl)-1H-1,2,3-triazol-4- | |
| | yl]methyl}amino)quinoline-3-carbonitrile | |
| 1044 | 8-bromo-4-[(3-chloro-4-fluorophenyl)amino]-6-({[3-(1- | 0.86 |
| | isopropylpiperidin-4-yl)-1H-pyrazol-5- | |
| | yl]methyl}amino)quinoline-3-carbonitrile | |
| 1045 | 8-bromo-4-[(3-chloro-4-fluorophenyl)amino]-6-({[3-(1- | 0.61 |
| | ethylpiperidin-4-yl)-1H-pyrazol-5- | |
| | yl]methyl}amino)quinoline-3-carbonitrile | |
| 1046 | tert-butyl 3-{4-[({8-chloro-4-[(3-chloro-4- | 0.3 |
| | fluorophenyl)amino]-3-cyanoquinolin-6-yl}amino)methyl]- | |
| | 1H-1,2,3-triazol-1-yl}-8-azabicyclo[3.2.1]octane-8- | |
| | carboxylate | |
| 1047 | 6-{[(5-bromopyridin-3-yl)methyl]amino}-8-chloro-4-[(3- | 0.47 |
| | chloro-4-fluorophenyl)amino]quinoline-3-carbonitrile | |
| 1048 | 8-chloro-4-[(3-chloro-4-fluorophenyl)amino]-6-({[5-(4- | 0.45 |
| | hydroxytetrahydro-2H-pyran-4-yl)pyridin-3- | |
| | yl]methyl}amino)quinoline-3-carbonitrile | |
| 1049 | 8-chloro-4-[(3-chloro-4-fluorophenyl)amino]-6-({[1-(8- | 0.42 |
| | isopropyl-8-azabicyclo[3.2.1]oct-3-yl)-1H-1,2,3-triazol-4- | |
| | yl]methyl}amino)quinoline-3-carbonitrile | |
| 1050 | 8-chloro-4-[(3-chloro-4-fluorophenyl)amino]-6-[({1-[8- | 0.046 |
| | (methylsulfonyl)-8-azabicyclo[3.2.1]oct-3-yl]-1H-1,2,3- | |
| | triazol-4-yl}methyl)amino]quinoline-3-carbonitrile | |
| 1051 | tert-butyl 5-[({8-chloro-4-[(3-chloro-4- | >3 |
| | fluorophenyl)amino]-3-cyanoquinolin-6-yl}amino)methyl]- | |
| | 3′,6′-dihydro-3,4′-bipyridine-1′(2′H)-carboxylate | |
| 1052 | 3-{4-[({8-chloro-4-[(3-chloro-4-fluorophenyl)amino]-3- | 0.023 |
| | cyanoquinolin-6-yl}amino)methyl]-1H-1,2,3-triazol-1-yl}- | |
| | N-ethyl-8-azabicyclo[3.2.1]octane-8-carboxamide | |
| 1053 | 6-({[1-(8-acetyl-8-azabicyclo[3.2.1]oct-3-yl)-1H-1,2,3- | 0.024 |
| | triazol-4-yl]methyl}amino)-8-chloro-4-[(3-chloro-4- | |
| | fluorophenyl)amino]quinoline-3-carbonitrile | |
| 1054 | 8-chloro-4-[(3-chloro-4-fluorophenyl)amino]-6-{[(1′- | 1.3 |
| | isopropyl-1′,2′,3′,6′-tetrahydro-3,4′-bipyridin-5- | |
| | yl)methyl]amino}quinoline-3-carbonitrile | |
| 1055 | 8-chloro-4-[(3-chloro-4-fluorophenyl)amino]-6-({[1-(8- | 0.008 |
| | propyl-8-azabicyclo[3.2.1]oct-3-yl)-1H-1,2,3-triazol-4- | |
| | yl]methyl}amino)quinoline-3-carbonitrile | |
| 1056 | 8-chloro-4-[(3-chloro-4-fluorophenyl)amino]-6-[(1′,2′,3′,6′- | 0.88 |
| | tetrahydro-3,4′-bipyridin-5-ylmethyl)amino]quinoline-3- | |
| | carbonitrile | |
| 1057 | 8-chloro-4-[(3-chloro-4-fluorophenyl)amino]-6-[({1-[8- | 0.011 |
| | (cyclopropylmethyl)-8-azabicyclo[3.2.1]oct-3-yl]-1H- | |
| | 1,2,3-triazol-4-yl}methyl)amino]quinoline-3-carbonitrile | |
| 1058 | 8-chloro-4-[(3-chloro-4-fluorophenyl)amino]-6-({[1-(8- | 0.07 |
| | isobutyl-8-azabicyclo[3.2.1]oct-3-yl)-1H-1,2,3-triazol-4- | |
| | yl]methyl}amino)quinoline-3-carbonitrile | |
| 1059 | 8-chloro-4-[(3-chloro-4-fluorophenyl)amino]-6-({[1-(8- | 0.014 |
| | formyl-8-azabicyclo[3.2.1]oct-3-yl)-1H-1,2,3-triazol-4- | |
| | yl]methyl}amino)quinoline-3-carbonitrile | |
| 1060 | 8-chloro-4-[(3-chloro-4-fluorophenyl)amino]-6-({[1-(4- | 0.045 |
| | hydroxy-4-methylcyclohexyl)-1H-1,2,3-triazol-4- | |
| | yl]methyl}amino)quinoline-3-carbonitrile | |
| 1061 | 8-chloro-4-[(3-chloro-4-fluorophenyl)amino]-6-({[5-(1- | 0.6 |
| | isopropylpiperidin-4-yl)pyridin-3- | |
| | yl]methyl}amino)quinoline-3-carbonitrile | |

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A compound of formula (I): 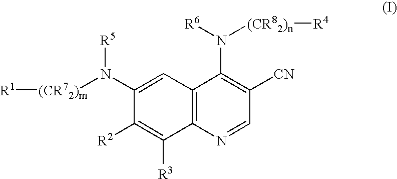
wherein:
R¹ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) N₃, e) OR⁹, f) NR¹⁰R¹¹, g) oxo, h) thioxo, i) S(O)ₚR⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl,
wherein any of o)-x) optionally is substituted with 1-4 R¹² groups;
R² is selected from the group consisting of:
a) H, b) halogen, c) CN, d) NO₂, e) OR⁹, f) NR¹⁰R¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₃₋₁₀ cycloalkyl, r) aryl, s) 3-10 membered cycloheteroalkyl, and t) heteroaryl,
wherein any of l)-t) optionally is substituted with 1-4 R¹² groups;
R³ is selected from the group consisting of:
a) H, b) halogen, c) CN, d) NO₂, e) OR⁹, f) NR¹⁰R¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₁₋₆ haloalkyl, r) C₃₋₁₀ cycloalkyl, s) aryl, t) 3-10 membered cycloheteroalkyl, and u) heteroaryl,
wherein any of l)-u) optionally is substituted with 1-4 R¹² groups;
R⁴ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) OR⁹, e) NR¹⁰R¹¹, f) oxo, g) thioxo, h) S(O)ₚR⁹, i) SO₂NR¹⁰R¹¹, j) C(O)R⁹, k) C(O)OR⁹, l) C(O)NR¹⁰R¹¹, m) Si(C₁₋₆ alkyl)₃, n) C₁₋₆ alkyl, o) C₂₋₆ alkenyl, p) C₂₋₆ alkynyl, q) C₁₋₆ alkoxy, r) C₁₋₆ alkylthio, s) C₁₋₆ haloalkyl, t) C₃₋₁₀ cycloalkyl, u) aryl, v) 3-10 membered cycloheteroalkyl, and w) heteroaryl,
wherein any of n)-w) optionally is substituted with 1-4 R¹² groups;
alternatively, R⁴ is selected from the group consisting of C₁₋₆ alkyl optionally substituted with 1-4 R¹² groups, C₁₋₆ haloalkyl, OR⁹, NR¹⁰R¹¹,C(O)OR⁹, C(O)NR¹⁰R¹¹, S(O)ₚR⁹, and N₃;
R⁵ and R⁶ at each occurrence independently are selected from the group consisting of:
a) H, b) C(O)R⁹, c) C(O)OR⁹, d) C(O)NR¹⁰R¹¹, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and 1) heteroaryl,
wherein any of e)-l) optionally is substituted with 1-4 R¹² groups;
R⁷ and R⁸ at each occurrence independently are selected from the group consisting of:
a) H, b) halogen, c) OR⁹, d) NR¹⁰R¹¹, e) C₁₋₆ alkyl, f) C₂₋₆ alkenyl, g) C₂₋₆ alkynyl, h) C₁₋₆ haloalkyl, and i) aryl;
alternatively, any two R⁷ or R⁸ groups and the carbon to which they are bonded may form a carbonyl group;
R⁹ at each occurrence is selected from the group consisting of:
a) H, b) C(O)R¹³, c) C(O)OR¹³, d) C(O)NR¹³R¹⁴, e) C₁₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl,
wherein any of e)-l) optionally is substituted with 1-4 R¹⁵ groups;
R¹⁰ and R¹¹ at each occurrence independently are selected from the group consisting of:
a) H, b) OR¹³ c) SO₂R¹³, d) C(O)R¹³, e) C(O)OR¹³, f) C(O)NR¹³R¹⁴, g) C₁₋₆ alkyl, h) C₂₋₆ alkenyl, i) C₂₋₆ alkynyl, k) C₁₋₆ haloalkyl, l) C₃₋₁₀ cycloalkyl, m) aryl, n) 3-10 membered cycloheteroalkyl, and o) heteroaryl;
wherein any of g)-o) optionally is substituted with 1-4 R¹⁵ groups;
R¹² at each occurrence independently is selected from the group consisting of:
a) halogen, b) CN, c) NO₂, d) N₃, e) OR⁹, f) NR¹⁰R¹¹, g) oxo, h) thioxo, i) S(O)ₚR⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl;
wherein any of o)-x) optionally is substituted with 1-4 R¹⁵ groups;
R¹³ and R¹⁴ at each occurrence independently are selected from the group consisting of:
a) H, b) C₁₋₆ alkyl, c) C₂₋₆ alkenyl, d) C₂₋₆ alkynyl, e) C₁₋₆ haloalkyl, f) C₃₋₁₀ cycloalkyl, g) aryl, h) 3-10 membered cycloheteroalkyl, and i) heteroaryl, wherein any of b)-j) optionally is substituted with 1-4 R¹⁵ groups;
R¹⁵ at each occurrence independently is selected from the group consisting of:
a) halogen, b) CN, c) NO₂, d) N₃, e) OH, f) O—C₁₋₆ alkyl, g) NH₂, h) NH(C₁₋₆ alkyl), i) N(C₁₋₆alkyl)₂, j) NH(aryl), k) NH(cycloalkyl), l) NH(heteroaryl), m) NH(cycloheteroalkyl), n) oxo, o) thioxo, p) SH, q) S(O)ₚ—C₁₋₆ alkyl, r) C(O)—C₁₋₆ alkyl, s) C(O)OH, t) C(O)O—C₁₋₆ alkyl, u) C(O)NH₂, v) C(O)NHC₁₋₆ alkyl, w) C(O)N(C₁₋₆ alkyl)₂, x) C₁₋₆ alkyl, y) C₂₋₆ alkenyl, z) C₂₋₆ alkynyl, aa) C₁₋₆ alkoxy, bb) C₁₋₆ alkylthio, cc) C₁₋₆ haloalkyl, dd) C₃₋₁₀ cycloalkyl, ee) aryl, ff) 3-10 membered cycloheteroalkyl, and gg) heteroaryl,
wherein any C₁₋₆ alkyl, C₂₋₆ alkenyl, C₂₋₆ alkynyl, C₃₋₁₀ cycloalkyl, aryl, 3-10 membered cycloheteroalkyl, or heteroaryl, alone as a part of another moiety, optionally is substituted with one or more moieties selected from the group consisting of halogen, CN, NO₂, OH, O—C₁₋₆ alkyl, NH₂, NH(C₁₋₆ alkyl), N(C₁₋₆ alkyl)₂, NH(aryl), NH(cycloalkyl), NH(heteroaryl), NH(cycloheteroalkyl), oxo, thioxo, SH, S(O)ₚ—C₁₋₆ alkyl, C(O)—C₁₋₆ alkyl, C(O)OH, C(O)O—C₁₋₆ alkyl, C(O)NH₂, C(O)NHC₁₋₆ alkyl, C(O)N—(C₁₋₆ alkyl)₂, C₁₋₆ alkyl, C₂₋₆ alkenyl, C₂₋₆ alkynyl, C₁₋₆ alkoxy, C₁₋₆ alkylthio, C₁₋₆ haloalkyl, C₃₋₁₀ cycloalkyl, aryl, 3-10 membered cycloheteroalkyl, and heteroaryl;
m is 0, 1, 2, 3, or 4;
n is 0 or 1; and
p is 0, 1, or 2;
or a pharmaceutically acceptable salt thereof,
provided that the compound of formula (I) does not comprise 4-(3-Chloro-4-fluoro-phenylamino)-7-methoxy-6-(4-morpholin-4-yl-butylamino)-quinoline-3-carbonitrile or 4-(3-Bromo-phenylamino)-6-(3-pyrrolidin-1-yl-propylamino)-quinoline-3-carbonitrile.

2. The compound according to claim 1, wherein R² is H.

3. The compound according to claim 1, wherein R² is C₁₋₆ alkylthio optionally substituted with NR¹⁰R¹¹.

4. The compound according to claim 3, wherein R² is SCH₂CH₂N(CH₃)₂.

5. The compound according to claim 1, wherein R³ is H.

6. The compound according to claim 1, wherein R³ is halogen.

7. The compound according to claim 6, wherein R³ is is Br.

8. The compound according to claim 6, wherein R³ is is Cl.

9. The compound according to claim 1, wherein R⁴ is phenyl.

10. The compound according to claim 9, wherein R⁴ is phenyl substituted with 1-2 halogens.

11. The compound according to claim 10, wherein R⁴ is phenyl substituted with Cl.

12. The compound according to claim 10, wherein R⁴ is phenyl substituted with F.

13. The compound according to claim 10, wherein R⁴ is phenyl substituted with Cl and F.

14. The compound according to claim 13, wherein R⁴ is 3-chloro-4-fluorophenyl.

15. The compound according to claim 1, wherein R¹ is a 5 or 6 membered heteroaryl.

16. The compound according to claim 15, wherein R¹ is imidazole.

17. The compound according to claim 15, wherein R¹ is triazole.

18. The compound according to claim 17, wherein R¹ is 1,2,3-triazole.

19. The compound according to claim 15, wherein R¹ is tetrazole.

20. The compound according to claim 15, wherein R¹ is pyridine.

21. The compound according to claim 15, wherein R¹ is N-oxypyridine.

22. The compound according to claim 1, wherein m is 1.

23. The compound according to claim 1, wherein n is 0.

24. The compound according to claim 1, wherein R⁵ is H.

25. The compound according to claim 1, wherein R⁵ is C₁₋₆ alkyl.

26. The compound according to claim 1, wherein R⁶ is H.

27. The compound according to claim 1, wherein R⁶ is C₁₋₆ alkyl.

28. A method of preventing or treating disease conditions mediated by Tpl-2 kinase in a mammal, comprising administering to the mammal a pharmaceutically effective amount of a compound according to claim 1, or a pharmaceutically acceptable salt thereof.

29. A method of alleviating a symptom of a disease mediated by Tpl-2 kinase in a mammal, comprising administering to the mammal a pharmaceutically effective amount of a compound according to claim 1, or a pharmaceutically acceptable salt thereof.

30. A method of preventing or treating an inflammatory disease in a mammal, comprising administering to the mammal a pharmaceutically effective amount of a compound according to claim 1, or a pharmaceutically acceptable salt thereof.

31. The method of claim 30, wherein the inflammatory disease is rheumatoid arthritis, juvenile rheumatoid arthritis, psoriatic arthritis, ankylosing spondylitis, or osteoarthritis.

32. A method of alleviating a symptom of an inflammatory disease in a mammal, comprising administering to the mammal a pharmaceutically effective amount of a compound according to claim 1, or a pharmaceutically acceptable salt thereof.

33. The method of claim 32, wherein the inflammatory disease is rheumatoid arthritis, juvenile rheumatoid arthritis, psoriatic arthritis, ankylosing spondylitis, or osteoarthritis.

34. A pharmaceutical composition comprising one or more compounds according to claim 1, or pharmaceutically salts thereof, and one or more pharmaceutically acceptable carriers.

35. A compound of formula (II): 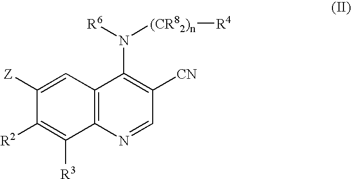
wherein:
Z is selected from the group consisting of halogen, C₁₋₆ alkyl optionally substituted with 1-4 R¹² groups, C₁₋₆ haloalkyl, OR⁹, NR¹⁰R¹¹, S(O)ₚR⁹, SO₂NR¹⁰R₁₁, C(O)R⁹, C(O)OR⁹, C(O)NR¹⁰R¹¹, and N₃;
R² is selected from the group consisting of:
a) H, b) halogen, c) CN, d) NO₂, e) OR⁹, f) NR¹⁰R¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₃₋₁₀ cycloalkyl, r) aryl, s) 3-10 membered cycloheteroalkyl, and t) heteroaryl,
wherein any of l)-t) optionally is substituted with 1-4 R¹² groups;
R³ is selected from the group consisting of:
a) H, b) halogen, c) CN, d) NO₂, e) OR⁹, f) NR¹⁰R¹¹, g) S(O)ₚR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₁₋₆ haloalkyl, r) C₃₋₁₀ cycloalkyl, s) aryl, t) 3-10 membered cycloheteroalkyl, and u) heteroaryl,
wherein any of l)-u) optionally is substituted with 1-4 R¹² groups;
R⁴ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) OR⁹, e) NR¹⁰R¹¹, f) oxo, g) thioxo, h) S(O)ₚR⁹, i) SO₂NR¹⁰R¹¹, j) C(O)R⁹, k) C(O)OR⁹, l) C(O)NR¹⁰R¹¹, m) Si(C₁₋₆alkyl)₃, n) C₁₋₆ alkyl, o) C₂₋₆ alkenyl, p) C₂₋₆ alkynyl, q) C₁₋₆ alkoxy, r) C₁₋₆ alkylthio, s) C₁₋₆ haloalkyl, t) C₃₋₁₀ cycloalkyl, u) aryl, v) 3-10 membered cycloheteroalkyl, and w) heteroaryl,
wherein any of n)-w) optionally is substituted with 1-4 R¹² groups;
alternatively, R⁴ is selected from the group consisting of C₁₋₆ alkyl optionally substituted with 1-4 R¹² groups, C₁₋₆ haloalkyl, OR⁹, NR¹⁰R¹¹,C(O)OR⁹, C(O)NR¹⁰R¹¹, S(O)ₚR⁹, and N₃;
R⁶ at each occurrence independently is selected from the group consisting of:
a) H, b) C(O)R⁹, c) C(O)OR⁹, d) C(O)NR¹⁰R¹¹, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl,
wherein any of e)-l) optionally is substituted with 1-4 R¹² groups;
R³ at each occurrence independently is selected from the group consisting of:
a) H, b) halogen, c) OR⁹, d) NR¹⁰R¹¹, e) C₁₋₆ alkyl, f) C₂₋₆ alkenyl, g) C₂₋₆ alkynyl, h) C₁₋₆ haloalkyl, and i) aryl;
alternatively, any two R³ groups and the carbon to which they are bonded may form a carbonyl group;
R⁹ at each occurrence is selected from the group consisting of:
a) H, b) C(O)R¹³, c) C(O)OR¹³, d) C(O)NR¹³R¹⁴, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl,
wherein any of e)-l) optionally is substituted with 1-4 R¹⁵ groups;
R¹⁰ and R¹¹ at each occurrence independently are selected from the group consisting of:
a) H, b) OR¹³, c) SO₂R¹³, d) C(O)R¹³, e) C(O)OR¹³, f) C(O)NR¹³R¹⁴, g) C₁₋₆ alkyl, h) C₂₋₆ alkenyl, i) C₂₋₆ alkynyl, k) C₁₋₆ haloalkyl, l) C₃₋₁₀ cycloalkyl, m) aryl, n) 3-10 membered cycloheteroalkyl, and o) heteroaryl;
wherein any of g)-o) optionally is substituted with 1-4 R¹⁵ groups;
R¹² at each occurrence independently is selected from the group consisting of:
a) halogen, b) CN, c) NO₂, d) N₃, e) OR⁹, f) NR¹⁰R¹¹, g) oxo, h) thioxo, i) S(O)ₚR⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl;
wherein any of o)-x) optionally is substituted with 1-4 R¹⁵ groups;
R¹³ and R¹⁴ at each occurrence independently are selected from the group consisting of:
a) H, b) C₁₋₆ alkyl, c) C₂₋₆ alkenyl, d) C₂₋₆ alkynyl, e) C₁₋₆ haloalkyl, f) C₃₋₁₀ cycloalkyl, g) aryl, h) 3-10 membered cycloheteroalkyl, and i) heteroaryl,
wherein any of b)-j) optionally is substituted with 1-4 R¹⁵ groups;
R¹⁵ at each occurrence independently is selected from the group consisting of:
a) halogen, b) CN, c) NO₂, d) N³, e) OH, f) O—C₁₋₆ alkyl, g) NH₂, h) NH(C₁₋₆ alkyl), i) N(C₁₋₆ alkyl)₂, j) NH(aryl), k) NH(cycloalkyl), l) NH(heteroaryl), m) NH(cycloheteroalkyl), n) oxo, o) thioxo, p) SH, q) S(O)ₚ—C₁₋₆ alkyl, r) C(O)—CO₁₋₆ alkyl, s) C(O)OH, t) C(O)O—C₁₋₆ alkyl, u) C(O)NH₂, v) C(O)NHC₁₋₆ alkyl, w) C(O)N(C₁₋₆ alkyl)₂, x) C₁₋₆ alkyl, y) C₂₋₆ alkenyl, z) C₂₋₆ alkynyl, aa) C₁₋₆ alkoxy, bb) C₁₋₆ alkylthio, CC) C₁₋₆ haloalkyl, dd) C₃₋₁₀ cycloalkyl, ee) aryl, ff) 3-10 membered cycloheteroalkyl, and gg) heteroaryl,
wherein any C₁₋₆ alkyl, C₂₋₆ alkenyl, C₂₋₆ alkynyl, C₃₋₁₀ cycloalkyl, aryl, 3-10 membered cycloheteroalkyl, or heteroaryl, alone as a part of another moiety, optionally is substituted with one or more moieties selected from the group consisting of halogen, CN, NO₂, OH, O—C₁₋₆ alkyl, NH₂, NH(C₁₋₆ alkyl), N(C₁₋₆ alkyl)₂, NH(aryl), NH(cycloalkyl), NH(heteroaryl), NH(cycloheteroalkyl), oxo, thioxo, SH, S(O)ₚ—C₁₋₆ alkyl, C(O)—C₁₋₆ alkyl, C(O)OH, C(O)O—C₁₋₆ alkyl, C(O)NH₂, C(O)NHC₁₋₆ alkyl, C(O)N(C₁₋₆ alkyl)₂, C₁₋₆ alkyl, C₂₋₆ alkenyl, C₂₋₆ alkynyl, C₁₋₆ alkoxy, C₁₋₆ alkylthio, C₁₋₆ haloalkyl, C₃₋₁₀ cycloalkyl, aryl, 3-10 membered cycloheteroalkyl, and heteroaryl;
n is 0 or 1; and
p is 0, 1, or 2,
or a pharmaceutically acceptable salt thereof.
